(19) United States
(12) Patent Application Publication
(10) Pub. No.: US 2022/0249475 A1
(43) Pub. Date: Aug. 11, 2022

(54) COMPOUNDS AND METHOD FOR TREATING CYTOKINE RELEASE SYNDROME

(71) Applicant: Rigel Pharmaceuticals, Inc., South San Francisco, CA (US)

(72) Inventors: Vanessa Taylor, South San Francisco, CA (US); Sarkiz Issakani, South San Francisco, CA (US); Chi Young, South San Francisco, CA (US)

(73) Assignee: Rigel Pharmaceuticals, Inc., South San Francisco, CA (US)

(21) Appl. No.: 17/592,779

(22) Filed: Feb. 4, 2022

Related U.S. Application Data
(63) Continuation of application No. PCT/US2020/045402, filed on Aug. 7, 2020.
(60) Provisional application No. 62/884,457, filed on Aug. 8, 2019.

(51) Int. Cl. A61K 31/505; A61P 37/06; A61K 31/4439; A61K 45/06; A61K 31/573; A61K 31/675; A61K 31/5377; A61K 31/519; A61K 31/4985

(52) U.S. Cl. CPC A61K 31/505; A61P 37/06; A61K 31/4439; A61K 45/06; A61K 31/4985; A61K 31/675; A61K 31/5377; A61K 31/519; A61K 31/573

(57) ABSTRACT

Disclosed herein are embodiments of a method for treating or preventing cytokine release syndrome (CRS). In certain embodiments, the method comprises administering a compound, or a salt, solvate, prodrug or pharmaceutical composition thereof, to a subject experiencing, or at risk of developing, CRS. The compound may be a kinase inhibitor, such as a JAK inhibitor and/or an IRAK inhibitor, and/or the compound may have a structure according to Formulas I, III, IV or VII. And the method may comprise administering the compound to a subject who is has received, is currently receiving, and/or will be receiving a cell therapy.

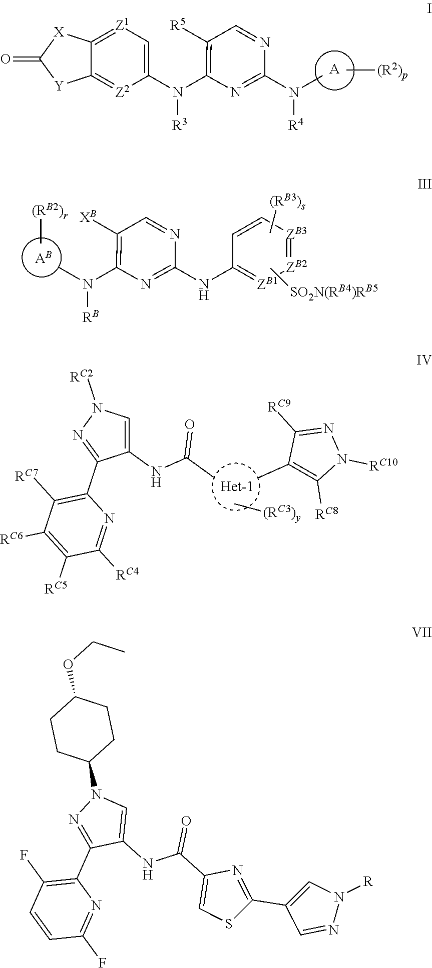

### CROSS REFERENCE TO RELATED APPLICATION

This application is a continuation of International Application No. PCT/US2020/045402, filed on Aug. 7, 2020, which was published in English under PCT Article 21(2), which in turn claims the benefit under 35 U.S.C. § 119(e) of the earlier filing date of U.S. Provisional Application No. 62/884,457, filed on Aug. 8, 2019. Both applications are incorporated herein by reference in their entireties.

### FIELD

The present application concerns compounds, and salt, solvates and/or prodrugs thereof, and pharmaceutical compositions containing them, and methods of using the compounds, and salts, solvates, prodrugs and/or compositions thereof, to treat cytokine release syndrome.

### BACKGROUND

Cytokine release syndrome (CRS) is a potentially life-threatening condition that may result from a variety of factors, including severe viral infections such as influenza, administration of antibodies that are used for immunotherapy, such as cancer immunotherapy, and non-protein-based cancer drugs such as oxaliplatin and lenalidomide. Immunotherapy can involve high levels of immune activation that exceed naturally occurring immune activation levels, and CRS is a non-antigen specific toxicity that can occur as a result. As immune-based therapies become more potent, CRS is becoming increasing diagnosed. CRS has also been observed in the setting of haploidentical donor stem cell transplantation, and graft-versus-host disease. Shimabukuro-Vornhagen et al., Journal for ImmunoTherapy of Cancer 6:56 (2018). CRS is associated with elevated circulating levels of several cytokines including interleukin (IL)-6 and interferon γ. Lee et al., Blood 124(2):188-195 (10 July 2014; Epub 29 May 2014).

CRS typically is clinically observed when significant numbers of lymphocytes and/or myeloid cells are activated and release inflammatory cytokines. The cytokine release may be induced by chemo- or biotherapy, and/or may be associated with therapeutic antibody treatments, such as immunotherapy, for example, for cancer treatment. Exemplary immunotherapies that may result in CRS include, but are not limited to, therapies where the cells express recombinant receptors, such as chimeric antigen receptors (CARs) and/or other transgenic receptors such as T cell receptors (TCRs). CRS induced by CAR T therapy generally occurs within days of T cell infusion at the peak of CAR T cell expansion. Giavridis et al., Nat Med. 24(6):731-738 (June 2018; Epub 28 May 2018). Examples of CART therapy that can induce CRS include axicabtagene ciloleucel (marketed as YESCARTA®) and tisagenlecleucel (marketed as KYMRIAH®).

Highly elevated interleukin 6 (IL-6) levels have been observed in patients with CRS and also in murine models of the disease, indicating that IL-6 may have a role in CRS pathophysiology. Shimabukuro-Vornhagen, J Immunother Cancer 6(1), 56 (2018). IL-6 can signal via two different modes. Classical IL-6 signaling involves binding of IL-6 to a membrane-bound IL-6 receptor. However, the IL-6 receptor does not possess intracellular signaling domains. Instead, after soluble IL-6 binds to membrane-bound IL-6 receptors, the IL-6/IL-6 receptor complex binds to membrane-bound gp130, which initiates signaling through its intracellular domain. In trans-signaling, IL-6 binds to a soluble form of the IL-6 receptor, which is typically cleaved from the cell surface by metalloproteinases. The resulting soluble IL-6/IL-6 receptor complex binds to gp130 and therefore can also induce signaling in cell types that do not express membrane bound IL-6 receptors.

IL-6 contributes to many of the key symptoms of CRS. Via trans-signaling, IL-6 leads to characteristic symptoms of severe CRS, i.e. vascular leakage, and activation of the complement and coagulation cascade inducing disseminated intravascular coagulation (DIC). In addition, IL-6 likely contributes to cardiomyopathy that is often observed in patients with CRS by promoting myocardial dysfunction. In a murine model, CRS developed within 2-3 days of CAR T cell infusion and could be lethal. Giavridis et al., Nat Med. 24(6): 731-738 (2018). CRS symptoms may start within minutes or hours of the start of antibody treatment, and can include a fever, which may reach or exceed 40° C., nausea, fatigue, headache, tachycardia, hypotension, rash, shortness of breath, and/or myalgias. However, in certain cases, additional and potentially more serious complications may develop, including cardiac dysfunction, adult respiratory distress syndrome, neurological toxicity, renal and/or hepatic failure, and/or disseminated intravascular coagulation.

The National Cancer Institute Common Terminology Criteria for Adverse Events (CTCAE v. 5.0, pub. Nov. 27, 2017) includes a grading system for CRS.

Grade 1: Fever with or without constitutional symptoms.

Grade 2: Hypotension responding to fluids; hypoxia responding to <40% O_{2.}

Grade 3: Hypotension managed with one pressor; hypoxia requiring ≥40% O_{2.}

Grade 4: Life-threatening consequences; urgent intervention indicated.

Grade 5: Death.

Other conditions associated with elevated cytokines, including CRS, also have been identified, such as syndromes associate with viral infections, such as COVID-19. One such syndrome is Acute Respiratory Syndrome or “ARDS,” wherein fluid builds up in lung alveoli, limiting the amount of oxygen that can be absorbed.

### SUMMARY

Disclosed herein are embodiments of a method for treating or preventing damage caused by elevated levels of inflammatory cytokines, such as in CRS. In some embodiments, the method comprises administering to a subject experiencing CRS, or at risk of developing CRS, an effective amount of a compound. The compound may be a kinase modulator and/or inhibitor, such as a JAnus Kinases (JAK) and/or Interleukin Receptor-Associated Kinase (IRAK) modulator and/or inhibitor. The compound may be a pyrimidine diamine compounds and/or may have a structure according to Formulas I or III, or a salt, solvate, N-oxide and/or prodrug thereof. Alternatively, the compound may be a pyrazole compound and/or may have a structure according to Formulas IV or VII, or a salt, solvate, N-oxide and/or prodrug thereof.

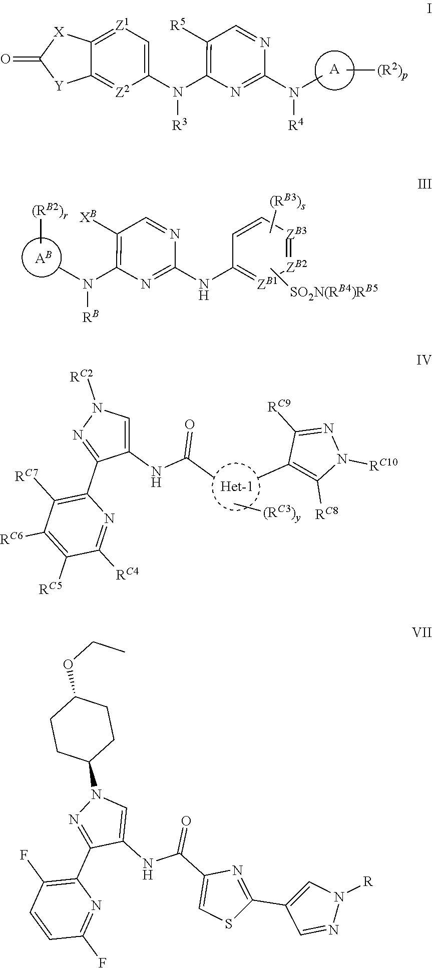

With respect to Formula I, X and Y are each independently O, S, S(O), SO₂ or NR¹; each R¹ is independently for each occurrence H, C₁₋₆alkyl, C(O)——C₁₋₆alkyl, CO₂—C₁₋₆alkyl or R⁵⁰; each R⁵⁰ is C(R⁹)₂—O—R¹⁰ or C(R⁹)₂—S—R¹⁰; each R⁹ is independently for each occurrence H, C₁₋₆alkyl, C₆₋₁₀aryl or C₇₋₁₆arylalkyl; or alternatively, two R⁹, together with the carbon to which they are attached, form a C₃₋₈cycloalkyl group or a 3-8 membered heterocycloaliphatic; R¹⁰ is R^{a} or —P(O)(OR¹¹)₂; each R¹¹ is independently for each occurrence R^{a} or a monovalent cationic group; or two R¹¹, together with the atoms to which they are attached, form a 4-8 membered cyclic phosphate group, or two R¹¹ together represent a divalent cationic group; ring A is a C₆₋₁₀aryl or a 5-10 membered heteroaryl; each R² is independently for each occurrence H, W, R^{b}, R^{a} substituted with one or more of the same or different R^{a}and/or R^{b}, —OR^{e} substituted with one or more of the same or different R^{a}and/or R^{b}, —SR^{e} substituted with one or more of the same or different R^{a} and/or R^{b}, —C(O)R^{e} substituted with one or more of the same or different R^{a} and/or R^{b}, —N(R^{a})R^{e} where R^{e} is substituted with one or more of the same or different R^{a} and/or R^{b}, —S(O)₂R^{e} substituted with one or more of the same or different R^{a} and/or R^{b}, —B(OR^{a})₂, —B(N(R^{c})₂)₂, —(C(R^{a})₂)ₘ—R^{b}, —O—(C(R^{a})₂)ₘ—R^{b}, —S—(C(R^{a})₂)ₘ—R^{b}, —O—(C(R^{b})₂)ₘ—R^{a}, —N(R^{a})—(C(R^{a})₂)ₘ—R^{b}, —O—(CH₂)ₘ—CH((CH₂)ₘ—R^{b})R^{b}, —C(O)N(R^{a})—(C(R^{a})₂)ₘ—R^{b}, —O—(C(R^{a})₂)ₘ—C(O)N(R^{a})13 (C(R^{a})₂)ₘ—R^{b}, —N((C(R^{a})₂)ₘ—R^{b})₂, —S—C(R^{a})₂)ₘ—C(O)N(R^{a})—(C(R^{a}) ₂)ₘ—R^{b}, —N(R^{a})—C(O)—N(R^{a})—(C(R^{a})₂)ₘ—R^{b}, —N(R^{a})—C(O)—(C(R^{a})₂)ₘ—C(R^{a})(R^{b})₂ or —N(R^{a})—(C(R^{a})₂)ₘ—C(O)—N(R^{a})—(C(R^{a})₂)ₘ—R^{b}; p is 0, 1, 2,3 or 4; each m is 1, 2 or 3; each n is 0, 1, 2 or 3; or two R² groups, taken together with the atom or atoms to which they are attached, combine to form a 4-10 membered partially or fully saturated mono or bicyclic ring, optionally containing one or more heteroatoms and optionally substituted with one or more R^{a} and/or R^{b}; Z¹ and Z² are each independently CH, CR² or N; R³ is H, C₁₋₆alkyl or R⁵⁰; R⁴ is H, C₁₋₆alkyl or R⁵⁰; and R⁵ is halo, —CN, C₁₋₆alkyl, alkynyl, hydroxy, C₁₋₆alkoxy, nitro, —N(R^{a})₂, —C(O)N(R^{a})₂, —CO₂R^{a} ₒᵣ —C(O)R^{a}. Additionally, each R^{a} is independently for each occurrence H, deuterium, C₁₋₆alkyl, C₃₋₈cycloalkyl, C₄₋₁₁cycloalkylalkyl, C₆₋₁₀aryl, C₇₋₁₆arylalkyl, 2-6 membered heteroalkyl, 3-10 membered heterocycloaliphatic, 4-11 membered heterocycloaliphaticalkyl, 5-15 membered heteroaryl or 6-16 membered heteroarylalkyl; each R^{b} is independently for each occurrence ═O, —OR^{a}, —O—(C(R^{a})₂)ₘ—OR^{a}, haloC₁₋₃alkyloxy, ═S, —SR^{a}, ═NR^{a}, ═NOR^{a}, —N(R^{c})₂, halo, —CF₃, —CN, —NC, —OCN, —SCN, —NO, —NO₂, ═N₂, —N₃, —S(O)R^{a}, —S(O)₂R^{a}, —SO₃R^{a}, —S(O)N(R^{a})₂, —OS(O)R^{a}, —OS(O)₂R^{a}, —OSO₃R^{a}, —OS(O)₂N(R^{a})₂, —C(O)R^{a}, —CO₂R^{a}, —C(O)N(R^{a})₂, —C(NR^{a})—N(R^{a})₂, —C(NOH)-R^{a}, —C(NOH)—N(R^{a})₂, —OC(O)R^{a}, —OC(O)OR^{a}, —OC(O)N(R^{c})₂, —OC(NH)—N(R^{a})₂, —OC(NR^{a})—N(R^{a})₂, —[N(R^{a})C(O)]ₙR^{a}, —[N(R^{a})C(O)]ₙOR^{a}, —[N(R^{a})C(O)]ₙN(R^{c})₂ or —[N(R^{a})C(NR^{a})]ₙ, —N(R^{a})₂; each R^{c} is independently for each occurrence R^{a}, or, alternatively, two R^{c} are taken together with the nitrogen atom to which they are bonded to form a 3 to 10-membered heterocycloaliphatic or a 5-10 membered heteroaryl which may optionally include one or more of the same or different additional heteroatoms and which is optionally substituted with one or more of the same or different R^{a} and/or R^{d} groups; each R^{d} is ═O, —OR^{a}, haloC₁₋₃alkyloxy, C₁₋₆alkyl, ═S, —SR^{a}, ═NR^{a}, ═NOR^{a}, —N(R^{a})₂, halo, —CF₃, —CN, —NC, —OCN, —SCN, —NO, —NO₂, ═N₂, —N₃, —S(O)R^{a}, —S(O₂)R^{a}, —SO₃R^{a}, —S(O)N(R^{a})₂, —S(O)₂N(R^{a})₂, —OS(O)R^{a}, —OS(O)₂R^{a}, —OSO₃R^{a}, —OS(O)₂N(R^{a})₂, —C(O)R^{a}, —CO₂R^{a}, —C(O)N(R^{a})₂, —C(NR^{a})N(R^{a})₂, —C(NOH)R^{a}, —C(NOH)N(R^{a})₂, —OCO₂R^{a}, —OC(O)N(R^{a})₂, —OC(NR^{a})N(R^{a})₂, —[N(R^{a})C(O)]ₙR^{a}, —(C(R^{a})₂)ₙ—OR^{a}, —N(R^{a})—S(O)₂R^{a}, —C(O)—C₁₋₆haloalkyl, —S(O)₂C₁₋₆ haloalkyl, —OC(O)R^{a}, —O(C(R^{a})₂)ₘ—OR^{a}, —S(C(R^{a})₂)ₘ—OR^{a}, —N(R^{a})C₁₋₆ haloalkyl, —P(O)(OR^{a})₂, —N(R^{a})—(C(R^{a})₂)ₘ—OR^{a}, —[N(R^{a})C(O)]ₙOR^{a}, —[N(R^{a})C(O)]ₙN(R^{a})₂, —[N(R^{a})C(NR^{a})]ₙN(R^{a})₂ or —N(R^{a})C(O)C₁₋₆alkyl; or two R^{d}, taken together with the atom or atoms to which they are attached, combine to form a 3-10 membered partially or fully saturated mono or bicyclic ring, optionally containing one or more heteroatoms and optionally substituted with one or more R^{a}; and each R^{e} is independently for each occurrence C₁₋₆alkyl, C₃₋₈cycloalkyl, C₄₋₁₁ cycloalkylalkyl, C₆₋₁₀aryl, C₇₋₁₆arylalkyl, 2-6 membered heteroalkyl, 3-10 membered heterocycloaliphatic, 4-11 membered heterocycloaliphaticalkyl, 5-15 membered heteroaryl or 6-16 membered heteroarylalkyl.

In some embodiments, the compound may have a structure according to Formulas IA or IA3

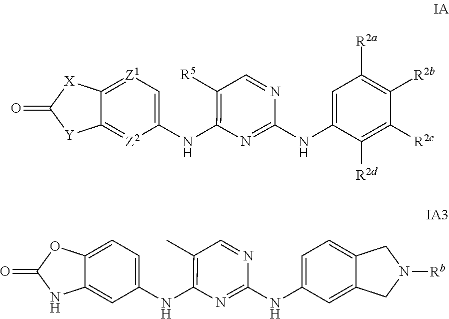

or a salt, solvate, N-oxide or prodrug thereof. With respect to Formula IA, each of R^{2a}, R^{2b}, R^{2c} and R^{2d} is independently for each occurrence as previous defined for R². In certain embodiments, R⁵ is halo or C₁₋₆alkyl, such as F or CH₃, and/or Z¹ is CH, C-halo or C—C₁₋₆alkyl, and Z² is CH. And with respect to Formula IA3, R^{b} is OH, C₁₋₆alkyl, —CO₂C₁₋₆alkyl, —C(O)C₁₋₆alkyl or —S(O)₂C₁₋₆alkyl.

In other embodiments, the compound has a structure according to Formula IB or Formula II

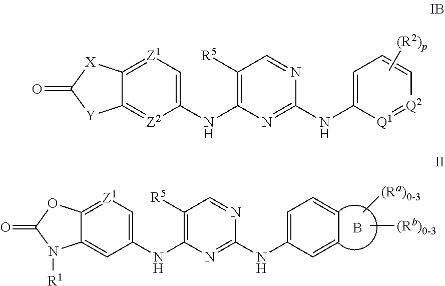

or a salt, solvate, N-oxide or prodrug thereof. With respect to Formula IB, Q¹ and Q² are each independently N or CH provided at least one of Q¹ and Q² is N. And in some embodiments, X and Y are each independently O or NR¹; each R¹ is independently for each occurrence H, C₁₋₆alkyl or R⁵⁰; p is 0, 1, 2 or 3; and/or R⁵ is halo, —CN, C₁₋₆alkyl, nitro, —N(R^{a})₂, —C(O)N(R^{a})₂, —CO₂R^{a} or —C(O)R^{a}. And with respect to Formula II, ring B, together with the two phenyl ring atoms to which it is attached, forms a 5, 6 or 7-membered ring, optionally containing 1, 2 or 3 heteroatoms independently selected from N(R^{c}), O and S; each R^{a} is C₁₋₆alkyl; and each R^{b} is independently for each occurrence ═O, —OR^{a}, haloC₁₋₃alkyloxy, —SR^{a}, —N(R^{a})₂, halo, —CF₃, —CN, —S(O)₂N(R^{c})₂, —S(O)₂R^{a}, —C(O)R^{a}, —CO₂R^{a}, —C(O)N(R^{c})₂, —N(R^{a})—S(O)₂R^{a} or —C(R^{a})₂—N(R^{c})₂. In certain embodiments, Z¹ is CH, C-halo, or C-C₁₋₆alkyl.

In alternative embodiments, the compound is a pyrimidine diamine compound according to Formula III

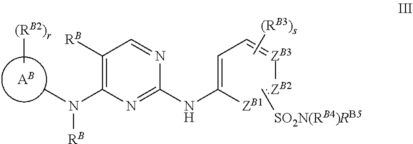

or a salt, solvate, N-oxide or prodrug thereof. With respect to Formula III, X^{B} is alkyl, alkoxy, amino, carboxyl, carboxyl ester, cyano, halo, nitro, alkenyl, or alkynyl; R^{B} is hydrogen, alkyl, alkenyl, alkynyl, or cycloalkyl; ring A^{B} is aryl, heteroaryl, cycloalkyl, cycloalkenyl or heterocyclic, wherein ring A^{B} is not indolyl or benzimidazolyl; r is 0, 1, 2 or 3; each R^{B2} independently is alkyl, alkoxy, amino, aryl, aryloxy (i.e. aryl-O—), cyano, cycloalkyl, cycloalkoxy, heteroaryl, heteroaryloxy, heterocyclic, heterocyclyloxy, aminoacyl, carboxyl, carboxyl ester, carbonate ester, sulfonyl, oxo, nitro or halo, preferably alkoxy; Z^{B1}, Z^{B2}, and Z^{B3} each independently is carbon or nitrogen, wherein if Z^{B1} is nitrogen then Z^{B2} and Z^{B3} are carbon, if Z^{B2} is nitrogen then Z^{B1} and Z^{B3} are carbon, and if Z^{B3} is nitrogen then Z^{B1} and Z^{B2} are carbon, wherein if Z^{B1}, Z^{B2}, or Z^{B3} is nitrogen then SO₂R^{B4}R^{B5} is not attached to the nitrogen; s is 0, 1, 2 or 3; each R^{B3} independently is hydrogen, alkyl, alkoxy, or cycloalkyl, halo, or heterocyclic; and each of R^{B4} and R^{B5} independently is hydrogen, alkyl, acyl or M⁺, wherein M⁺ is a metal counterion selected from K⁺, Na⁺, Li⁺ or ⁺N(R⁶)₄, wherein R^{B6} is hydrogen or alkyl, and the nitrogen of SO₂NR^{B4}R^{B5} is N⁻; or R^{B4} or R^{B5} is a divalent counterion selected from Ca²⁺, mg²+, and Ba²⁺, and the nitrogen of SO₂NR^{B4}R^{B5} is N. In certain embodiments, the compound is selected from

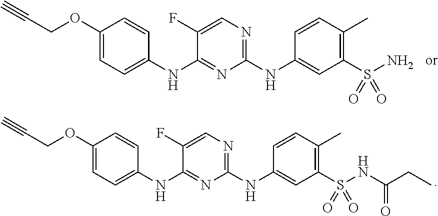 [SMILES: C#CCOc1ccc(Nc2nc(Nc3ccc(C)c(S(N)(=O)=O)c3)ncc2F)cc1 ; C#CCOc1ccc(Nc2nc(Nc3ccc(C)c(S(=O)(=O)NC(=O)CC)c3)ncc2F)cc1]

In alternative embodiments, the compound is a pyrazole compound and may have a Formula IV or a salt, prodrug, solvate and/or N-oxide thereof.

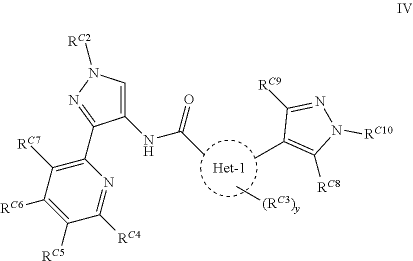

With respect to Formula IV, Het-1 is 5-membered heteroaryl, such as thiazolyl or furanyl; y is from 1 to 2; R^{C2} is H, aliphatic, heteroaliphatic, heterocycloaliphatic, aryl, amide, heterocyclyl or araliphatic, and may be H alkyl, haloalkyl or cycloalkyl, such as H or alkyl; each R^{C3} independently is H or aliphatic; R^{C4}, R^{C5}, R^{C6} and R^{C7} are each independently H, aliphatic, heteroaliphatic, alkoxy, heterocyclyl, aryl, araliphatic, —O—heterocyclyl, hydroxyl, haloalkyl, halogen, nitro, cyano, carboxyl, carboxyl ester, acyl, amide, amino, sulfonyl, sulfonamide, sulfanyl or sulfinyl; R^{C8} and R^{C9} are each independently H, aliphatic, heteroaliphatic, aryl, heterocyclyl, sulfonyl, nitro, halogen, haloalkyl, carboxyl ester, cyano or amino, such as H, halogen, haloalkyl, or alkyl; and R^{C10} is H, aliphatic, alkoxy, heteroaliphatic, carboxyl ester, araliphatic, NO₂, CN, OH, haloalkyl, acyl, alkyl phosphate or alkylphosphonate, such as H, alkyl, alkyl phosphate or alkyl phosphonate. In some embodiments, each of R^{C4}, R^{C6}, and R^{C7} independently is H, halo, alkyl or haloalkyl, and may be H or F. And in certain embodiments, R^{C5} is H, halo, aliphatic, alkoxy, heterocyclyl, or —O—heterocyclyl, and may be R^{C5} is H, F, CF₃, methoxy, —O—CH₂C(CH₃)₂OH, morpholin-4-yl, 1-methylpiperidin-4-yl, or —O—(oxetan-3-yl).

In some embodiments, the compound has a structure, or a salt, prodrug, solvate and/or N-oxide thereof, according to Formulas V or VI

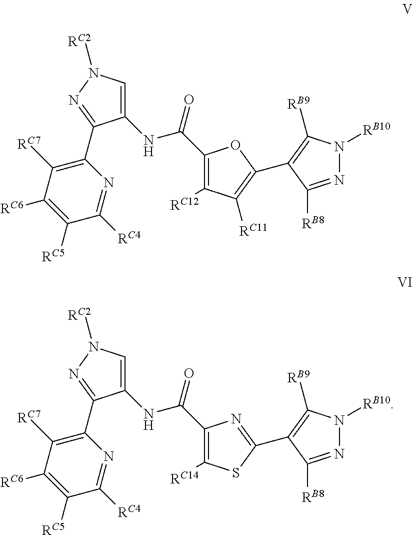

With respect to Formulas V and VI, each of R^{C11}, R^{C12} and R^{C14} independently is H or aliphatic.

In alternative embodiments, the compound is a pyrazole compound according to Formula VII or a salt, prodrug, solvate and/or N-oxide thereof.

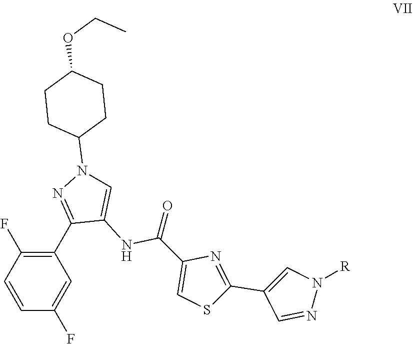

or a salt, solvate or N-oxide thereof. With respect to Formula VII, R is H, aliphatic, acyl, heterocyclyl, carboxyl ester, amide, alkyl phosphoramidate, or alkyl phosphate. In some embodiments, R is not H, or alternatively, R is H and the compound is a salt. In other embodiments, R is alkyl, acyl, carboxyl ester, amide, nonaromatic heterocyclyl, alkyl phosphoramidate, or alkyl phosphate. A person of ordinary skill in the art understands that compounds where R is not H may act a prodrug of the compound where R is H, for example, when administered to a subject.

In any embodiments of the method, the subject may not exhibit a sign or symptom of CRS and/or may be at risk of developing CRS. In such embodiments, administering the compound substantially prevents the onset of CRS, or prevents the onset of grade 2 or higher CRS.

In other embodiments, the subject exhibits at least one sign or symptom of CRS and may exhibit at least one sign or symptom of grade 1 CRS. Alternatively, the subject may exhibit at least one sign or symptom of grade 2 or higher CRS, such as grade 3 or higher CRS. The compound may be administered within 24 hours of the onset of the sign or symptom, and/or administering the compound may ameliorate the sign or symptom of CRS, compared to the severity of the sign or symptom prior to administration of the compound, such as reducing the grade of CRS from 4 to 3, 2 or 1, or from 3, to 2 or 1, or from 2 to 1. Alternatively, CRS symptoms are substantially reduced to below grade 1 level, such that the subject no longer experiences symptoms associated with CRS. In some embodiments the sign or symptom is a fever and may be a fever of 40° C. or higher.

High levels of inflammatory cytokines also have been reported during COVID-19 infection. These cytokines include interferons, interleukins, chemokines, colony-stimulating factors, and tumor necrosis factors and contribute to the symptoms of coronavirus infection. One consequence of a cytokine storm associated with COVID-19 infection is acute organ injury, which in the case of lung injury, can progress to a more severe form called acute respiratory distress syndrome. Accordingly, the present compounds can be administered to patients infected with COVID-19 to block, ameliorate or treat inflammation associated with the condition and its treatment.

The method may comprise administering to a subject that has previously be administered a first therapy for which harmful inflammatory cytokine production, such as CRS, is a known, suspected, or potential side effect. Administration of the first therapy may be initiated from greater than zero to 10 days prior to administration of the compound. Alternatively, the compound may be administered to a subject who will be, or is concurrently being, administered a first therapy for which CRS is a known, suspected, and/or potential side effect. In any embodiments, the first therapy may comprise a cell therapy, including, but not limited to, chimeric antigen receptor (CAR)-expressing therapy and/or a transgenic receptor therapy. Cell-free antibodies are also known to elicit this syndrome, particularly those that activate T-cells, including, but not limited to, CAMPATH 1-H, blinatumomab, and/or rituximab. In some embodiments, the method may further comprise administering a second therapeutic agent, for example, a steroid, an anti-viral, an anti-inflammatory agent, an immunosuppressant, or a combination thereof. The steroid may be a corticosteroid, such as, for example, dexamethasone or prednisone, or a combination thereof. In any embodiments, the compound may be administered substantially simultaneously with the second therapeutic agent, or the compound and second therapeutic agent may be administered sequentially in any order.

The foregoing and other objects, features, and advantages of the invention will become more apparent from the following detailed description.

### DETAILED DESCRIPTION

### I. Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

When chemical structures are depicted or described, unless explicitly stated otherwise, all carbons are assumed to include hydrogen so that each carbon conforms to a valence of four. For example, in the structure on the left-hand side of the schematic below there are nine hydrogen atoms implied. The nine hydrogen atoms are depicted in the right-hand structure.

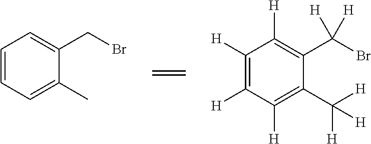

Sometimes a particular atom in a structure is described in textual formula as having a hydrogen or hydrogen atoms, for example —CH₂CH₂—. It will be understood by a person of ordinary skill in the art that the aforementioned descriptive techniques are common in the chemical arts to provide brevity and simplicity to description of organic structures.

If a group R is depicted as “floating” on a ring system, as for example in the group:

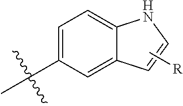

then, unless otherwise defined, a substituent R can reside on any atom of the fused bicyclic ring system, so long as a stable structure is formed that conforms to standard valence conditions as understood by a person of ordinary skill in the art. In the example depicted, the R group can reside on an atom in either the 5-membered or the 6-membered ring of the indolyl ring system, including the heteroatom by replacing the explicitly recited hydrogen, but excluding the atom carrying the bond with the [symbol] symbol and the bridging carbon atoms.

When there are more than one such depicted “floating” groups, as for example in the formulae:

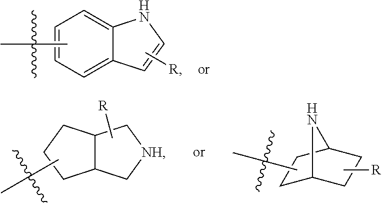

where there are two groups, namely, the R and the bond indicating attachment to a parent structure; then, unless otherwise defined, each “floating” group can reside on any atoms of the ring system, again assuming each replaces a depicted, implied, or expressly defined hydrogen on the ring system and a chemically stable compound would be formed by such an arrangement.

When a group R is depicted as existing on a ring system containing saturated carbons, for example as in the formula:

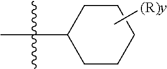

where, in this example, y can be more than one, and assuming each R replaces a currently depicted, implied, or expressly defined hydrogen on the ring; then, unless otherwise defined, two R's can reside on the same carbon. A simple example is when R is a methyl group. The depicted structure can exist as a geminal dimethyl on a carbon of the depicted ring (an “annular” carbon). In another example, two R's on the same carbon, including that same carbon, can form a ring, thus creating a spirocyclic ring (a “spirocyclyl” group) structure. For example, shown below two Rs can form a piperidine ring in a spirocyclic arrangement with the cyclohexane, as

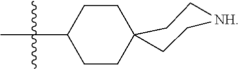 [SMILES: *C1CCC2(CCNCC2)CC1]

A person of ordinary skill in the art will appreciate that the definitions may be combined to further describe a particular compound. For example, hydroxyaliphatic refers to an aliphatic group substituted with an hydroxy (—OH) group, and haloalkylaryl refers to an aryl group substituted with an alkyl group, where the alkyl group too is substituted with a halogen, and where the point of attachment to the parent structure is via the aryl moiety since aryl is the base name of the substituent.

As used herein, the term “substituted” refers to all subsequent modifiers in a term, for example in the term “substituted arylC₁₋₈salkyl,” substitution may occur on the “C₁₋₈salkyl” portion, the “aryl” portion or both portions of the arylC₁₋₈alkyl group. Also by way of example, alkyl includes substituted cycloalkyl groups.

“Substituted,” when used to modify a specified group or moiety, means that at least one, and perhaps two or more, hydrogen atoms of the specified group or moiety is independently replaced with the same or different substituent groups as defined below. In a particular embodiment, a group, moiety or substituent may be substituted or unsubstituted, unless expressly defined as either “unsubstituted” or “substituted.” Accordingly, any of the groups specified herein may be unsubstituted or substituted. In particular embodiments, the substituent may or may not be expressly defined as substituted, but is still contemplated to be optionally substituted. For example, an “alkyl” or a “pyrazolyl” moiety may be unsubstituted or substituted, but an “unsubstituted alkyl” or an “unsubstituted pyrazolyl” is not substituted.

“Substituents” or “substituent groups” for substituting for one or more hydrogen atoms on saturated carbon atoms in the specified group or moiety are, unless otherwise specified, —R⁶⁰, halo, ═O, —OR⁷⁰, —SR⁷⁰, —N(R⁸⁰)₂, haloalkyl, perhaloalkyl, —CN, —NO₂, ═N₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —P(O)(O⁻)₂(M⁺)₂, —P(O)(O⁻)₂M²⁺, —P(O)(OR⁷⁰)O⁻M⁺, —P(O)(OR⁷⁰)₂, —C(O)R⁷⁰, —C(S)R⁷⁰, —C(NR⁷⁰)R^{70, —CO}₂⁻M⁺, —CO₂R⁷⁰, —C(S)OR⁷⁰, —C(O)N(R₈₀)₂, —C(NR₇₀)(R₈₀)₂, OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR₇₀C(S)OR⁷⁰, —NR⁷⁰C(O)N(R⁸⁰)₂, —NR⁷⁰C(NR⁷⁰)R⁷⁰ or —NR⁷⁰(NR⁷⁰)N(R⁸⁰)², where R⁶⁰ is C₁₋₁₀aliphatic, heteroaliphatic, or cycloaliphatic, typically, C₁₋₆aliphatic, more typically C₁₋₆alkyl, where R⁶⁰ optionally may be substituted; each R⁷⁰ is independently for each occurrence hydrogen or R⁶⁰; each R⁸⁰ is independently for each occurrence R⁷⁰ or alternatively, two R⁸⁰ groups, taken together with the nitrogen atom to which they are attached, form a 3- to 7-membered heterocycloaliphatic, which optionally includes from 1 to 4 of the same or different additional heteroatoms selected from O, N and S, of which N optionally has R⁷⁰ substitution, such as H or C₁-C₃alkyl substitution; and each M⁺ is a counter ion with a net single positive charge. Each M⁺ is independently for each occurrence, for example, an alkali metal ion, such as K⁺, Na⁺, Li⁺; an ammonium ion, such as ⁺N(R⁷⁰)₄; a protonated amino acid ion, such as a lysine ion , or an arginine ion; or an alkaline metal earth ion, such as [Ca²⁺]_{0.5}, [Mg²⁺]_{0.5}, or [Ba²⁺]_{0.5} (a subscript “0.5” means, for example, that one of the counter ions for such divalent alkali earth ions can be an ionized form of a compound of the invention and the other is a typical counter ion such as chloride, or two ionized compounds can serve as counter ions for such divalent alkali earth ions, or alternatively, a doubly ionized compound can serve as the counter ion for such divalent alkali earth ions). As specific examples, —N(R⁸⁰)₂ includes —NH₂, —NH-alkyl, —NH-pyrrolidin-3-yl, N-pyrrolidinyl, N-piperazinyl, 4N-methyl-piperazin-1-yl, N-morpholinyl and the like. Any two hydrogen atoms on a single carbon also can be replaced with, for example, ═O, ═NR⁷⁰, ═N—OR⁷⁰, ═N₂ or ═S.

Substituent groups for replacing hydrogen atoms on unsaturated carbon atoms in groups containing unsaturated carbons are, unless otherwise specified, —R⁶⁰, halo, —O⁻M⁺, —OR⁷⁰, —SR⁷⁰, —S⁻M⁺, —N(R⁸⁰)₂, perhaloalkyl, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —PO₃⁻²M²⁺, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)N(R⁸⁰)₂, —OC(O)R⁷⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)N(R⁸⁰)₂, —NR⁷⁰ C(NR⁷⁰)R⁷⁰ or —NR⁷⁰(NR⁷⁰)N(R⁸⁰)₂, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

Substituent groups for replacing hydrogen atoms on nitrogen atoms in groups containing such nitrogen atoms are, unless otherwise specified, —R⁶⁰, —O⁻M⁺, —OR⁷⁰, —SR⁷⁰, —S⁻M⁺, —N(R⁸⁰)₂, perhaloalkyl, —CN, —NO, —NO₂, —S(O)₂R⁷⁰, —SO₃⁻M⁺, —SO₃R⁷⁰, —OS(O)₂R⁷⁰, —OSO₃M⁺, —OSO₃R⁷⁰, —PO₃²⁻(M⁺)₂, —PO₃² M²⁺, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, −OCO₂R⁷⁰, —OC(S)OR⁷⁰, —NR⁷⁰C(O)R⁷⁰, —NR⁷⁰C(S)R⁷⁰, —NR⁷⁰CO₂R⁷⁰, —NR⁷⁰C(S)OR⁷⁰, —NR⁷⁰C(O)N(R⁸⁰)₂, —NR⁷⁰C(NR⁷⁰)R⁷⁰ or —NR⁷⁰C(NR⁷⁰)N(R⁸⁰)₂, where R⁶⁰, R⁷⁰, R⁸⁰ and M⁺ are as previously defined.

In one embodiment, a group that is substituted has 1 substituent, 2 substituents, substituents, or 4 substituents.

Additionally, in embodiments where a group or moiety is substituted with a substituted substituent, the nesting of such substituted substituents is limited to three, thereby preventing the formation of polymers. Thus, in a group or moiety comprising a first group that is a substituent on a second group that is itself a substituent on a third group, which is attached to the parent structure, the first (outermost) group can only be substituted with unsubstituted substituents. For example, in a group comprising -(aryl-1)-(aryl-2)-(aryl-3), aryl-3 can only be substituted with substituents that are not themselves substituted.

The term “acute respiratory distress syndrome” or “ARDS” refers to a syndrome characterized by a severe shortness of breath, labored and unusually rapid breathing, low blood pressure, confusion and extreme tiredness. This syndrome can be diagnosed based on a PaO2/FiO2 ratio of less than 300 mmHg despite a PEEP of more than 5 cm H2O (Fan et al. JAMA. 319: 698-71).

ARDS occurs when fluid builds up in lung alveoli. The fluid prevents the lungs from filling with enough air, limiting the amount of oxygen that reaches the bloodstream which, in turn, deprives the organs of the oxygen they need to function. The symptoms of ARDS can vary in intensity, depending on its cause and severity. Severe shortness of breath—the hallmark of ARDS—usually develops within a few hours to a few days after the COVID-19 infection. Many people who develop ARDS do not survive, and the risk of death increases with age and severity of illness. Of the patients that survive ARDS, some completely recover while others have lasting damage to their lungs.

“Acyl” refers to the group —C(O)R, where R is H, aliphatic, heteroaliphatic, heterocyclic or aromatic. Exemplary acyl moieties include, but are not limited to, —C(O)H, —C(O)alkyl, —C(O)C₁-C₆alkyl, —C(O)C₁-C₆ haloalkyl, —C(O)cycloalkyl, —C(O)alkenyl, —C(O)cycloalkenyl, —C(O)aryl, —C(O)heteroaryl, or —C(O)heterocyclyl. Specific examples include —C(O)H, —C(O)Me, —C(O)Et, or —C(O)cyclopropyl.

“Aliphatic” refers to a substantially hydrocarbon-based group or moiety. An aliphatic group or moiety can be acyclic, including alkyl, alkenyl, or alkynyl groups, cyclic versions thereof, such as cycloaliphatic groups or moieties including cycloalkyl, cycloalkenyl or cycloalkynyl, and further including straight- and branched-chain arrangements, and all stereo and position isomers as well. Unless expressly stated otherwise, an aliphatic group contains from one to twenty-five carbon atoms (C₁₋₂₅); for example, from one to fifteen (C₁₋₁₅), from one to ten (C₁₋₁₀), from one to six (C₁₋₆), or from one to four carbon atoms (C₁₋₄) for a saturated acyclic aliphatic group or moiety, from two to twenty-five carbon atoms (C₂₋₂₅); for example, from two to fifteen (C₂₋₁₅), from two to ten (C₂₋₁₀), from two to six (C₂₋₆), or from two to four carbon atoms (C₂₋₄) for an unsaturated acyclic aliphatic group or moiety, or from three to fifteen (C₃₋₁₅) from three to ten (C₃₋₁₀), from three to six (C₃₋₆), or from three to four (C₃₋₄) carbon atoms for a cycloaliphatic group or moiety. An aliphatic group may be substituted or unsubstituted, unless expressly referred to as an “unsubstituted aliphatic” or a “substituted aliphatic.” An aliphatic group can be substituted with one or more substituents (up to two substituents for each methylene carbon in an aliphatic chain, or up to one substituent for each carbon of a —C═C— double bond in an aliphatic chain, or up to one substituent for a carbon of a terminal methine group).

“Alkoxy” refers to the group —OR, where R is a substituted or unsubstituted alkyl or a substituted or unsubstituted cycloalkyl group. In certain examples R is a C₁₋₆ alkyl group or a C₃₋₆cycloalkyl group. Methoxy (—OCH₃) and ethoxy (—OCH₂CH₃) are exemplary alkoxy groups. In a substituted alkoxy, R is substituted alkyl or substituted cycloalkyl, examples of which include haloalkoxy groups, such as —OCF₂H, or —OCF₃.

“Alkyl” refers to a saturated aliphatic hydrocarbyl group having from 1 to 25 (C₁₋₂₅) or more carbon atoms, more typically 1 to 10 (C₁₋₁₀) carbon atoms such as 1 to 8 (C₁₋₈) carbon atoms, 1 to 6 (C₁₋₆) carbon atoms or 1 to 4 (C₁₋₄) carbon atoms. An alkyl moiety may be substituted or unsubstituted. This term includes, by way of example, linear and branched hydrocarbyl groups such as methyl (CH₃), ethyl (—CH₂CH₃), n-propyl (—CH₂CH₂CH₃), isopropyl (—CH(CH₃)₂), n-butyl (—CH₂CH₂CH₂CH₃), isobutyl (—CH₂CH₂(CH₃)₂), sec-butyl (—CH(CH₃)(CH₂CH₃), t-butyl (—C(CH₃)₃), n-pentyl (—CH₂CH₂CH₂CH₂CH₃), and neopentyl (—CH₂C(CH₃)3). As used herein, “lower alkyl” means (C₁-C₈) alkyl.

“Amino” refers to the group —NH₂, —NHR, or —NRR, where each R independently is selected from aliphatic, heteroaliphatic, aromatic, including both aryl and heteroaryl, or heterocycloaliphatic, or two R groups together with the nitrogen attached thereto form a heterocyclic ring. Examples of such heterocyclic rings include those wherein two R groups together with the nitrogen to which they are attached form a —(CH₂)₂₋₅-ring optionally interrupted by one or two additional heteroatom groups, such as O, S or N(R^{g}) such as in the groups

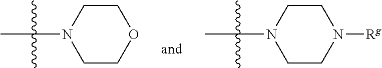

wherein R^{g} is R⁷⁰, —C(O)R⁷⁰, —C(O)OR⁶⁰ or —C(O)N(R⁸⁰)₂.

“Amide” or “carboxamide” refers to the group —N(R)acyl, or —C(O)amino, where R is hydrogen, heteroaliphatic, aromatic, or aliphatic, such as alkyl, particularly C₁₋₆alkyl.

“Aromatic” refers to a cyclic, conjugated group or moiety of, unless specified otherwise, from 5 to 15 ring atoms having a single ring (e.g., phenyl, pyridinyl, or pyrazolyl) or multiple condensed rings in which at least one ring is aromatic (e.g., naphthyl, indolyl, or pyrazolopyridinyl), that is at least one ring, and optionally multiple condensed rings, have a continuous, delocalized π-electron system. Typically, the number of out of plane π-electrons corresponds to the Hiickel rule (4n+2). The point of attachment to the parent structure typically is through an aromatic portion of the condensed ring system. For example,

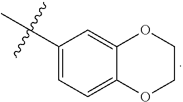 [SMILES: *c1ccc2c(c1)OCCO2]

However, in certain examples, context or express disclosure may indicate that the point of attachment is through a non-aromatic portion of the condensed ring system. For example,

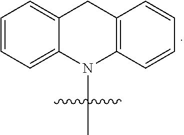 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g. S, O, N, P, or Si), such as in a heteroaryl group or moiety. Unless otherwise stated, an aromatic group may be substituted or unsubstituted.

“Aryl” refers to an aromatic carbocyclic group of, unless specified otherwise, from 6 to 15 carbon atoms having a single ring (e.g., phenyl) or multiple condensed rings in which at least one ring is aromatic multiple condensed rings in which at least one ring is aromatic (e.g., 1,2,3,4-tetrahydroquinoline, benzodioxole, and the like) providing that the point of attachment is through an aromatic portion of the ring system. If any aromatic ring portion contains a heteroatom, the group is heteroaryl and not aryl. Aryl groups may be, for example, monocyclic, bicyclic, tricyclic or tetracyclic. Unless otherwise stated, an aryl group may be substituted or unsubstituted.

“Araliphatic” refers to an aryl group attached to the parent via an aliphatic moiety. Araliphatic includes aralkyl or arylalkyl groups such as benzyl and phenylethyl.

“Carboxyl” or “carboxylic acid” refers to —CO₂H,

“Carboxylate” refers to —C(O)O⁻ or salts thereof.

“Carboxyl ester” or “carboxyate ester” refers to the group —C(O)OR, where R is aliphatic, heteroaliphatic, cyclicaliphatic, heterocyclic, and aromatic, including both aryl and heteroaryl.

“Combination” refers to two or more components that are administered such that the effective time period of at least one component overlaps with the effective time period of at least one other component. A combination, or a component thereof, may be a composition. In some embodiments, effective time periods of all components administered overlap with each other. In an exemplary embodiment of a combination comprising three components, the effective time period of the first component administered may overlap with the effective time periods of the second and third components, but the effective time periods of the second and third components independently may or may not overlap with one another. In another exemplary embodiment of a combination comprising three components, the effective time period of the first component administered overlaps with the effective time period of the second component, but not that of the third component; and the effective time period of the second component overlaps with those of the first and third components. A combination may be a composition comprising the components, a composition comprising one or more components and another separate component (or components) or composition(s) comprising the remaining component(s), or the combination may be two or more individual components. In some embodiments, the two or more components may comprise the same component administered at two or more different times, two or more different components administered substantially simultaneously or sequentially in any order, or a combination thereof.

The term “COVID-19” refers to a coronavirus COVID-19 (previously known as 2019-nCoV) which was first identified in Wuhan, China. The term “COVID-19-associated ARDS” refers to ARDS that is caused by COVID-19 infection. Patients having COVID-19-associated ARDS may have been diagnosed as having a COVID-19 infection, may have been exposed to another person having a COVID19 infection, or may be suspected of having a COVID-19 infection based on their symptoms.

“Cyano” refers to the group —CN.

“Cycloaliphatic” refers to a cyclic aliphatic group having a single ring (e.g., cyclohexyl), or multiple rings, such as in a fused, bridged or spirocyclic system, at least one of which is aliphatic. Typically, the point of attachment to the parent structure is through an aliphatic portion of the multiple ring system. Cycloaliphatic includes saturated and unsaturated systems, including cycloalkyl, cycloalkenyl and cycloalkynyl. A cycloaliphatic group may contain from three to twenty-five carbon atoms; for example, from three to fifteen, from three to ten, or from three to six carbon atoms. Unless otherwise stated, a cycloaliphatic group may be substituted or unsubstituted. Exemplary cycloaliphatic groups include, but are not limited to, cyclopropyl, cyclobutyl, cyclopentyl, cyclohexyl, cycloheptyl, cyclopentenyl, or cyclohexenyl. As used herein, lower cycloalkyl refers to C₃₋₈cycloalkyl.

“Halo,” “halide” or “halogen” refers to fluoro, chloro, bromo or iodo.

“Haloalkyl” refers to an alkyl moiety as defined herein that is substituted with one or more halogens. Exemplary haloalkyl moieties include —CH₂F, —CHF₂ and —CF₃.

“Heteroaliphatic” refers to an aliphatic compound or group having at least one heteroatom and at least one carbon atom, i.e., one or more carbon atoms from an aliphatic compound or group comprising at least two carbon atoms, has been replaced with an atom having at least one lone pair of electrons, typically nitrogen, oxygen, phosphorus, silicon, or sulfur. For example, a heteroalkyl moiety is a heteroaliphatic moiety where the base aliphatic moiety is an alkyl as defined herein. Heteroaliphatic compounds or groups may be substituted or unsubstituted, branched or unbranched, chiral or achiral, and/or acyclic or cyclic, such as a heterocycloaliphatic group.

“Heteroaryl” refers to an aromatic group or moiety of, unless specified otherwise, from 5 to 15 ring atoms comprising at least one carbon atom and at least one heteroatom, such as N, S, O, P, or Si. A heteroaryl group or moiety may comprise a single ring (e.g., pyridinyl, pyrimidinyl or pyrazolyl) or multiple condensed rings (e.g., indolyl, benzopyrazolyl, or pyrazolopyridinyl). Heteroaryl groups or moiety may be, for example, monocyclic, bicyclic, tricyclic or tetracyclic. Unless otherwise stated, a heteroaryl group or moiety may be substituted or unsubstituted.

“Heterocyclyl,” “heterocyclo” and “heterocycle” refer to both aromatic and non-aromatic ring systems, and more specifically refer to a stable three- to fifteen-membered ring moiety comprising at least one carbon atom, and typically plural carbon atoms, and at least one, such as from one to five, heteroatoms. The heteroatom(s) may be nitrogen, phosphorus, oxygen, silicon or sulfur atom(s). The heterocyclyl moiety may be a monocyclic moiety, or may comprise multiple rings, such as in a bicyclic or tricyclic ring system, provided that at least one of the rings contains a heteroatom. Such a multiple ring moiety can include fused or bridged ring systems as well as spirocyclic systems; and any nitrogen, phosphorus, carbon, silicon or sulfur atoms in the heterocyclyl moiety can be optionally oxidized to various oxidation states. For convenience, nitrogens, particularly, but not exclusively, those defined as annular aromatic nitrogens, are meant to include their corresponding N-oxide form, although not explicitly defined as such in a particular example. Thus, for a compound having, for example, a pyridinyl ring, the corresponding pyridinyl-N-oxide is included as another compound of the invention, unless expressly excluded or excluded by context. In addition, annular nitrogen atoms can be optionally quaternized. Heterocycle includes heteroaryl moieties, where the heterocylyl moieties are aromatic, and heterocycloaliphatic moieties, such as heterocycloalkyl, heterocycloalkenyl, or heterocycloalkynyl, which are heterocyclyl rings that are partially or fully saturated. Examples of heterocyclyl groups include, but are not limited to, azetidinyl, oxetanyl, acridinyl, benzodioxolyl, benzodioxanyl, benzofuranyl, carbazoyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, quinolinyl, isoquinolinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, dihydropyridinyl, tetrahydropyridinyl, pyridinyl, pyrazinyl, pyrimidinyl, pyridazinyl, oxazolyl, oxazolinyl, oxazolidinyl, triazolyl, isoxazolyl, isoxazolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzoxazolyl, furyl, diazabicycloheptane, diazapane, diazepine, tetrahydrofuryl, tetrahydropyranyl, thienyl, benzothieliyl, thiamorpholinyl, thiamorpholinyl sulfoxide, thiamorpholinyl sulfone, dioxaphospholanyl, and oxadiazolyl.

“Hydroxyl” refers to the group —OH.

“Nitro” refers to the group —NO₂.

“Oxo” refers to the group ═O (double bond O).

“Phosphate” refers to the group —O—P(O)(OR′)₂, where each —OR′ independently is —OH; —O-aliphatic, such as —O-alkyl or —O-cycloalkyl; —O-aromatic, including both —O-aryl and —O-heteroaryl; —O-aralkyl; or —OR′ is —O⁻M⁺, where M⁺ is a counter ion with a single positive charge. Each M⁺ may be an alkali ion, such as K^{t}, Na^{t}, Li^{t}; an ammonium ion, such as ⁺N(R″)₄ where each R″ independently is H, aliphatic, heterocyclyl or aryl; or an alkaline earth ion, such as [Ca^{2+]}_{0.5}, [Mg²⁺]_{0.5}, or [Ba²⁺]_{0.5}. Phosphonooxyalkyl refers to the group-alkyl-phosphate, such as, for example, —CH₂OP(O)(OH)₂, or a salt thereof, such as —CH₂OP(O)(O⁻Na⁺)₂, and (((dialkoxyphosphoryl)oxy)alkyl) refers to the dialkyl ester of a phosphonooxyalkyl group, such as, for example, —CH₂OP(O)(O-tert-butyl)₂.

“Phosphonate” refers to the group —P(O)(OR′)₂, where each —OR′ independently is —OH; —O-aliphatic such as —O-alkyl or —O-cycloalkyl; —O-aromatic, including both —O-aryl and —O-heteroaryl; or —O-aralkyl; or —OR′ is —O⁻M⁺, and M⁺ is a counter ion with a single positive charge. Each M⁺ is a positively charged counterion and may be, by way of example, an alkali metal ion, such as K⁺, Na⁺, Li⁺; an ammonium ion, such as ⁺N(R″)₄ where each R″ independently is H, aliphatic, heterocyclyl or aryl; or an alkaline earth metal ion, such as [Ca²⁺]_{0.5}, [Mg²⁺]_{0.5}, or [Ba²⁺]_{0.5}. Phosphonoalkyl refers to the group -alkyl-phosphonate, such as, for example, —CH₂P(O)(OH)₂, or —CH₂P(O)(O0⁻Na^{t})₂, and ((dialkoxyphosphoryl)alkyl) refers to the dialkyl ester of a phosphonoalkyl group, such as, for example, —CH₂P(O)(O-tert-butyl)₂.

“Phosphoramidate” refers to the group —O—P(O)(OR′)(N(R′)₂), where each R′ independently is H, aliphatic, such as alkyl, aryl, or aralkyl, or —OR′ is —O⁻M⁺, and where M^{t} is a counter ion with a single positive charge. Each M^{t} may be an alkali ion, such as K^{t}, Na^{t}, Li^{t}; an ammonium ion, such as ⁺N(R″)4 where each R″ independently is H, aliphatic, such as alkyl, hydroxyalkyl, or a combination thereof, heterocyclyl, or aryl; or an alkaline earth ion, such as [Ca²⁺]_{0.5}, [Mg²⁺]_{0.5}, or [Ba²⁺]_{0.5}. Alkyl phosphoramidate refers to the group -alkyl-phosphoramidate, such as, for example, —CH₂O—P(O)(OR′)(N(R′₂)) or —CH₂(CH₃)O—P(O)(OR′)(N(R′2)), such as, —CH₂O—P(O)(O-phenyl)[NHC(CH₃)CO₂isopropyl], or —CH₂OP(O)(OH)(N(H)alkyl), or a salt thereof, such as —CH₂OP(O)(O⁻Na⁺)(N(H)alkyl).

“Patient” or “Subject” refers to mammals and other animals, particularly humans. Thus, disclosed methods are applicable to both human therapy and veterinary applications.

“Pharmaceutically acceptable excipient” refers to a substance, other than the active ingredient, that is included in a formulation of the active ingredient. As used herein, an excipient may be incorporated within particles of a pharmaceutical composition, or it may be physically mixed with particles of a pharmaceutical composition. An excipient can be used, for example, to dilute an active agent and/or to modify properties of a pharmaceutical composition. Excipients can include, but are not limited to, antiadherents, binders, coatings, enteric coatings, disintegrants, flavorings, sweeteners, colorants, lubricants, glidants, sorbents, preservatives, adjuvants, carriers or vehicles. Excipients may be starches and modified starches, cellulose and cellulose derivatives, saccharides and their derivatives such as disaccharides, polysaccharides and sugar alcohols, protein, synthetic polymers, crosslinked polymers, antioxidants, amino acids or preservatives. Exemplary excipients include, but are not limited to, magnesium stearate, stearic acid, vegetable stearin, sucrose, lactose, starches, hydroxypropyl cellulose, hydroxypropyl methylcellulose, xylitol, sorbitol, maltitol, gelatin, polyvinylpyrrolidone (PVP), polyethyleneglycol (PEG), tocopheryl polyethylene glycol 1000 succinate (also known as vitamin E TPGS, or TPGS), carboxy methyl cellulose, dipalmitoyl phosphatidyl choline (DPPC), vitamin A, vitamin E, vitamin C, retinyl palmitate, selenium, cysteine, methionine, citric acid, sodium citrate, methyl paraben, propyl paraben, sugar, silica, talc, magnesium carbonate, sodium starch glycolate, tartrazine, aspartame, benzalkonium chloride, sesame oil, propyl gallate, sodium metabisulphite or lanolin.

An “adjuvant” is an excipient that modifies the effect of other agents, typically the active ingredient. Adjuvants are often pharmacological and/or immunological agents. An adjuvant may modify the effect of an active ingredient by increasing an immune response. An adjuvant may also act as a stabilizing agent for a formulation. Exemplary adjuvants include, but are not limited to, aluminum hydroxide, alum, aluminum phosphate, killed bacteria, squalene, detergents, cytokines, paraffin oil, and combination adjuvants, such as freund's complete adjuvant or freund's incomplete adjuvant.

“Pharmaceutically acceptable carrier” refers to an excipient that is a carrier or vehicle, such as a suspension aid, solubilizing aid, or aerosolization aid. Pharmaceutically acceptable carriers are conventional. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of one or more therapeutic compositions and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In some examples, the pharmaceutically acceptable carrier may be sterile to be suitable for administration to a subject (for example, by parenteral, intramuscular, or subcutaneous injection). In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

“Pharmaceutically acceptable salt” refers to pharmaceutically acceptable salts of a compound that are derived from a variety of organic and inorganic counter ions as will be known to a person of ordinary skill in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. “Pharmaceutically acceptable acid addition salts” are a subset of “pharmaceutically acceptable salts” that retain the biological effectiveness of the free bases while formed by acid partners. In particular, the disclosed compounds form salts with a variety of pharmaceutically acceptable acids, including, without limitation, inorganic acids such as hydrochloric acid, hydrobromic acid, hydroiodic acid, sulfuric acid, nitric acid, phosphoric acid, and the like, as well as organic acids such as formic acid, acetic acid, trifluoroacetic acid, propionic acid, glycolic acid, pyruvic acid, oxalic acid, malic acid, maleic acid, malonic acid, succinic acid, fumaric acid, tartaric acid, citric acid, benzoic acid, 3-(4-hydroxybenzoyl) benzoic acid, cinnamic acid, mandelic acid, benzene sulfonic acid, isethionic acid, salicylic acid, xinafoic acid, lactic acid, palmitic acid, alkylsulfonic acids (for example, methanesulfonic acid, ethanesulfonic acid, 1,2-ethane-disulfonic acid, 2-hydroxyethanesulfonic acid, etc.), arylsulfonic acids (for example, benzenesulfonic acid, 4-chlorobenzenesulfonic acid, 2-naphthalenesulfonic acid, 4-toluenesulfonic acid, camphorsulfonic acid, etc.), 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Pharmaceutically acceptable salts also include salts formed when an acidic proton present in the parent compound is either replaced by a metal ion (for example, an alkali metal ion, an alkaline earth metal ion or an aluminum ion) or coordinates with an organic base (for example, ethanolamine, diethanolamine, triethanolamine, N-methylglucamine, morpholine, piperidine, dimethylamine, diethylamine, triethylamine, ammonia, etc.).

“Pharmaceutically acceptable base addition salts” are a subset of “pharmaceutically acceptable salts” that are derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, tris(hydroxymethyl)aminomethane (Tris), ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purine, piperazine, piperidine, N-ethylpiperidine, polyamine resins, and the like. Exemplary organic bases are isopropylamine, diethylamine, tris(hydroxymethyl)aminomethane (Tris), ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.)

“Effective amount,” such as a therapeutically effective amount, refer to an amount of a compound sufficient to achieve a desired result, for example, to treat a specified disorder or disease, or to ameliorate or eradicate one or more of its symptoms and/or to prevent the occurrence of the disease or disorder. The amount of a compound which constitutes an “effective amount” will vary depending on the compound, the disease state and its severity, the age of the patient to be treated, and the like. The effective amount can be determined by a person of ordinary skill in the art. An appropriate “effective” amount in any individual case can be determined using any suitable technique, such as a dose escalation study.

“Prodrug” refers to compounds that are transformed in vivo to yield a biologically active compound, particularly the parent compound, for example, by hydrolysis in the gut or enzymatic conversion. Common examples of prodrug moieties include, but are not limited to, ester and amide forms of a compound having an active form bearing a carboxylic acid moiety. Examples of pharmaceutically acceptable esters suitable for use with the disclosed compounds include, but are not limited to, esters of phosphate groups and carboxylic acids, such as aliphatic esters, particularly alkyl esters (for example C₁₋₆alkyl esters). Other prodrug moieties include phosphate esters, such as —CH₂—O—P(O)(OR′)₂ or a salt thereof, wherein R′ is H or C₁₋₆alkyl. Acceptable esters also include cycloalkyl esters and arylalkyl esters such as, but not limited to benzyl. Examples of pharmaceutically acceptable amides of the disclosed compounds include, but are not limited to, primary amides, and secondary and tertiary alkyl amides (for example with between about one and about six carbons). Amides and esters of the disclosed compounds can be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated or alkylated such as benzyl and trityl ethers, as well as alkyl ethers, tetrahydropyranyl ethers, trialkylsilyl ethers (e.g., TMS or TIPPS groups) and allyl ethers.

“Spray-dried dispersion” refers to a single-phase dispersion of a compound or compounds in a polymer matrix. Typically, the compound or compounds are amorphous.

“Solvate” refers to a complex formed by combination of solvent molecules with molecules or ions of the solute. The solvent can be an organic compound, an inorganic compound, or a mixture of both. Some examples of solvents include, but are not limited to, methanol, ethanol, isopropanol, ethyl acetate, N,N-dimethylformamide, tetrahydrofuran, dimethylsulfoxide, and water. The compounds described herein can exist in un-solvated as well as solvated forms when combined with solvents, pharmaceutically acceptable or not, such as water, ethanol, and the like. Solvated and unsolvated forms of the presently disclosed compounds are within the scope of the embodiments disclosed herein.

“Subject” refers to humans and non-human subjects.

“Sulfanyl” refers to the group or —SH, —S-aliphatic, —S-heteroaliphatic, —S-cyclic, —S-heterocyclyl, including —S-aryl and —S-heteroaryl .

“Sulfinyl” refers to the group or moiety —S(O)H, —S(O)aliphatic, —S(O)heteroaliphatic, —S(O)cyclic, —S(O)heterocyclyl, including —S(O)aryl and —S(O)heteroaryl.

“Sulfonyl” refers to the group: -502H, —SO₂aliphatic, —SO₂ heteroaliphatic, —SO₂cyclic, —SO₂ heterocyclyl, including —SO₂aryl and —SO₂ heteroaryl.

“Sulfonamide” refers to the group or moiety —SO₂amino, or —N(R^{a})sulfonyl, where R^{c} is H, aliphatic, heteroaliphatic, cyclic, and heterocyclic, including aryl and heteroaryl.

“Treating” or “treatment” as used herein concerns treatment of CRS in a patient or subject, particularly a human experiencing CRS, and includes by way of example, and without limitation:

(i) inhibiting CRS, for example, arresting or slowing its development;

(ii) relieving CRS, for example, causing regression of CRS or a symptom thereof; or

(iii) stabilizing CRS, such as by preventing the CRS from increasing in grade and/or severity.

In the case of COVID-19-associated cytokine elevation, resulting in, for example, ARDS, successful treatment may include a decrease in shortness of breath, less labored or less rapid breathing, higher blood pressure, decreased confusion and/or a decrease tiredness. A treatment may be administered prophylactically, that is, before the onset of ARDS. A prophylactic treatment prevents ARDS and can be administered to patients that have or are suspected of having a COVID-19 infection, but without the severe symptoms of ARDS. For example, prophylactic treatment can be administered to patients that have a cough without the other symptoms of ARDS.

“Preventing” as used herein concerns reducing cytokine levels or their inflammatory effects to prevent CRS from occurring in a patient or subject, in particular, when such patient or subject is at risk of developing CRS but has not yet been diagnosed as having it.

As used herein, the terms “disease” and “condition” can be used interchangeably or can be different in that the particular malady or condition may not have a known causative agent (so that etiology has not yet been determined) and it is therefore not yet recognized as a disease but only as an undesirable condition or syndrome, where a more or less specific set of symptoms have been identified by clinicians.

The above definitions and the following general formulas are not intended to include impermissible substitution patterns (e.g., methyl substituted with 5 fluoro groups). Such impermissible substitution patterns are easily recognized by a person having ordinary skill in the art.

Any of the groups referred to herein may be optionally substituted by at least one, possibly two or more, substituents as defined herein. That is, a substituted group has at least one, possible two or more, substitutable hydrogens replaced by a substituent or substituents as defined herein, unless the context indicates otherwise or a particular structural formula precludes substitution.

A person of ordinary skill in the art will appreciate that compounds may exhibit the phenomena of tautomerism, conformational isomerism, geometric isomerism, and/or optical isomerism. For example, certain disclosed compounds can include one or more chiral centers and/or double bonds and as a consequence can exist as stereoisomers, such as double-bond isomers (i.e., geometric isomers), enantiomers, diasteromers, and mixtures thereof, such as racemic mixtures. Accordingly, compounds and compositions may be provided as individual pure enantiomers or diasteriomers, or as stereoisomeric mixtures, including racemic mixtures. In certain embodiments the compounds disclosed herein are synthesized in or are purified to be in substantially enantiopure form, such as in an 85% enantiomeric excess (e.e.), a 90% enantiomeric excess, a 95% enantiomeric excess, a 97% enantiomeric excess, a 98% enantiomeric excess, a 99% enantiomeric excess, or even in greater than a 99% enantiomeric excess, such as in a substantially enantiopure form. A person of ordinary skill in the art understands that in a compound comprising one or more asymmetric centers, one or both enantiomers or diastereomers are contemplated unless a specific enantiomer or diastereomer is shown or described.

As another example, certain disclosed compounds can exist in several tautomeric forms, including the enol form, the keto form, and mixtures thereof. As the various compound names, formulae and compound drawings within the specification and claims can represent only one of the possible tautomeric, conformational isomeric, optical isomeric, or geometric isomeric forms, a person of ordinary skill in the art will appreciate that the disclosed compounds encompass any tautomeric, conformational isomeric, optical isomeric, and/or geometric isomeric forms of the compounds described herein, as well as mixtures of these various different isomeric forms. In cases of limited rotation, e.g. around the amide bond or between two directly attached rings such as the pyrazolyl and pyridinyl rings, atropisomers are also possible and are also specifically included in the compounds of the invention.

In any embodiments, any or all hydrogens present in the compound, or in a particular group or moiety within the compound, may be replaced by a deuterium or a tritium. Thus, a recitation of alkyl includes deuterated alkyl, where from one to the maximum number of hydrogens present may be replaced by deuterium. For example, ethyl may be C₂H₅ or C₂H₅ where from 1 to 5 hydrogens are replaced by deuterium, such as in C₂DₓH₅₋ₓ.

### II. Compounds

Disclosed herein are compounds, prodrugs, corresponding salt and/or solvate forms, and methods of using these compounds, prodrugs, and salt/solvate forms for treating and/or preventing CRS. The compounds may be compounds that modulate JAnus Kinases (JAK) and/or Interleukin Receptor-Associated Kinase (IRAK) pathways, and/or may be kinase inhibitors, including, but not limited to, JAK inhibitors, such as JAK1, JAK2, JAK3 and/or JAK4 inhibitors; and/or IRAK inhibitors, such as IRAK1, IRAK2, IRAK3 and/or IRAK4 inhibitors. The compound may be a pyrimidine diamine compound, such as a compound according to Formula I or Formula III, or a pyrazole compound, such as a compound according to Formula IV.

A. Pyrimidine diamine compounds according to Formula I

In some embodiments, the compound is a pyrimidine diamine compound according to formula I

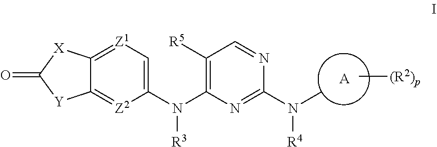

or a salt, solvate, prodrug and/or N-oxide thereof. With respect to formula I:

X and Y are each independently O, S, S(O), SO₂ or NR¹;

each R¹ is independently for each occurrence H, C₁₋₆alkyl, C(O)—C₁₋₆alkyl, CO₂—C₁₋₆alkyl or R⁵⁰;

each R⁵⁰ is C(R⁹)₂—O—R¹⁰ or C(R⁹)₂—S—R¹⁰;

each R⁹ is independently for each occurrence H, C₁₋₆alkyl, C₆₋₁₀aryl or C₇₋₁₆arylalkyl; or alternatively, two R⁹, together with the carbon to which they are attached, form a C₃₋₈cycloalkyl group or a 3-8 membered heterocycloaliphatic; R¹⁰ is R^{a} or —P(O)(OR¹¹)₂; each R¹¹ is independently for each occurrence R^{a} or a monovalent cationic group; or two R″, together with the atoms to which they are attached, form a 4-8 membered cyclic phosphate group, or two R″ together represent a divalent cationic group;

ring A is a C₆₋₁₀aryl or a 5-10 membered heteroaryl;

each R² is independently for each occurrence H, R^{e}, R^{b}, R^{e} substituted with one or more of the same or different R^{a} and/or R^{b}, —OR^{e} substituted with one or more of the same or different R^{a} and/or R^{b}, —SR^{e} substituted with one or more of the same or different R^{a} and/or R^{b}, —C(O)Re substituted with one or more of the same or different R^{a} and/or R^{b}, —N(R^{a})R^{e} where R^{a} is substituted with one or more of the same or different R^{a} and/or R^{b}, —S(O)₂R^{e} substituted with one or more of the same or different R^{a} and/or R^{b}, —B(OR^{a})₂, —B(N(R^{c})₂)₂, —(C(R^{a})₂)ₘ—R^{b}, —O—(C(R^{a})₂)ₘ—R^{b}, —S—(C(R^{a})₂)ₘ—R^{b}, —O—(C(R^{b})₂)ₘ—R^{a}, —N(R^{a})—-(C(R^{a})₂)ₘ—R^{b}, —O—(CH₂)ₘ—CH((CH₂)ₘ—R^{b})R^{b}, —C(O)N(R^{a})—(C(R^{a})₂)ₘ—R^{b}, —O—(C(R^{a})₂)ₘ—C(O)N(R^{a})—(C(R^{a})₂)ₘ—R^{b}, —N((C(R^{a})₂)R^{b})₂, —S—(C(R^{a})₂)ₘ—C(O)N(R^{a})—(C(R^{a})₂)ₘ—R^{b}, —N(R^{a})—C(O)—N(R^{a})—(C(R^{a})₂)ₘ—R^{b}, —N(R^{a})—C(O)—(C(R^{a})₂)ₘ—C(R^{a})(R^{b})₂ or —N(R^{a})—C(R^{a})₂)ₘ—C(O)—N(R^{a})—(C(R^{a})₂)ₘ—R^{b};

each R^{a} is independently for each occurrence H, deuterium, C₁₋₆alkyl, C₃₋₈cycloalkyl, C₄₋₁₁cycloalkylalkyl, C₆₋₁₀aryl, C₇₋₁₆arylalkyl, 2-6 membered heteroalkyl, 3-10 membered heterocycloaliphatic, 4-11 membered heterocycloaliphaticalkyl, 5-15 membered heteroaryl or 6-16 membered heteroarylalkyl;

each R^{b} is independently for each occurrence ═O, —OR^{a}, —O—(C(R^{a})₂)ₘ—OR^{a}, haloC₁₋₃alkyloxy, ═S, —SR^{a}, ═NR^{a}, ═NOR^{a}, —N(R^{c})₂, halo, —CF₃, —CN, —NC, —OCN, —SCN, —NO, —NO₂, ═N₂, —N₃, —S(O)R^{a}, —S(O)₂R^{a}, —SO₃R^{a}, —S(O)N(R^{c})₂, —OS(O)R^{a}, —OS(O)₂R^{a}, —OSO₃R^{a}, —OS(O)₂N(R^{c})₂, —C(O)R^{a}, —CO₂R^{a}, —C(O)N(R^{c})₂, —C(NR^{a})—N(R^{c})₂, —C(NOH)—R^{a}, —C(NOH)—N(R^{c})₂, —OC(O)R^{a}, —OC(O)OR^{a}, —OC(O)N(R^{c})₂, —OC(NH)—N(R^{c})₂, —OC(NR^{a})—N(R^{c})₂, —[N(R^{a})C(O)]ₙOR^{a}, —[N(R^{a})C(O)]ₙN(R^{c})₂ or —[N(R^{a})C(NR^{a})]ₙ—N(R^{c})₂;

each R^{c} is independently for each occurrence R^{a}, or, alternatively, two R^{c} are taken together with the nitrogen atom to which they are bonded to form a 3 to 10-membered heterocycloaliphatic or a 5-10 membered heteroaryl which may optionally include one or more of the same or different additional heteroatoms and which is optionally substituted with one or more of the same or different R^{a} and/or R^{d} groups;

each R^{d} is ═O, —OR^{a}, haloC₁₋₃alkyloxy,C₁₋₆alkyl, ═S, —SR^{a}, ═NR^{a}, ═NOR^{a}, —N(R^{a})₂, halo, —CF₃, —CN, —NC, —OCN, —SCN, —NO, —NO₂, ═N₂, —N₃, —S(O)R^{a}, —S(O₂)R^{a}, —SO₃R^{a}, —S(O)N(R^{a})₂, —S(O)₂N(R^{a})₂, —OS(O)R^{a}, —OS(O)₂R^{a}, —OSO₃R^{a}, —OS(O)₂N(R^{a})₂, —C(O)R^{a}, —CO₂R^{a}, —C(O)N(R^{a})₂, —C(NR^{a})N(R^{a})₂, —C(NOH)R^{a}, —C(NOH)N(R^{a})₂, —OCO₂R^{a}, —OC(O)N(R^{a})₂, —OC(NR^{a})N(R^{a})₂, —[N(R^{a})C(O)]ₙR^{a}, —(C(R^{a})₂)ₙ—OR^{a}, —N(R^{a}) —S(O)₂R^{a}, —C(O)-C₁₋₆ haloalkyl, —S(O)₂C₁₋₆ haloalkyl, —OC(O)R^{a}, —O(C(R^{a})₂)ₘ—OR^{a}, —S(C(R^{a})₂)ₘ—OR^{a}, —N(R^{a})C₁₋₆haloalkyl, —P(O)(OR^{a})₂, —N(R^{a})—(C(R^{a})₂)ₘ—OR^{a}, —[N(R^{a})C(O)]ₙOR^{a}, —[N(R^{a})C(O)]ₙN(R^{a})₂, —[N(R^{a})C(NR^{a})]ₙN(R^{a})₂ or —N(R^{a})C(O)C₁₋₆ haloalkyl; or two R^{d}, taken together with the atom or atoms to which they are attached, combine to form a 3-10 membered partially or fully saturated mono or bicyclic ring, optionally containing one or more heteroatoms and optionally substituted with one or more R^{a};

each R^{e} is independently for each occurrence C₁₋₆alkyl, C₃₋₈cycloalkyl, C₄₋₁₁ cycloalkylalkyl, C₆₋₁₀aryl, C₇₋₁₆arylalkyl, 2-6 membered heteroalkyl, 3-10 membered heterocycloaliphatic, 4-11 membered heterocycloaliphaticalkyl, 5-15 membered heteroaryl or 6-16 membered heteroarylalkyl;

p is 0,1,2,3 or 4;

each m is 1, 2 or 3;

each n is 0, 1, 2 or 3;

or two R² groups, taken together with the atom or atoms to which they are attached, combine to form a 4-10 membered partially or fully saturated mono or bicyclic ring, optionally containing one or more heteroatoms and optionally substituted with one or more R^{a} and/or R^{b};

Z¹ and Z² are each independently CH, CR² or N;

R³ is H, C₁₋₆alkyl or R⁵⁰;

R⁴ is H, C₁₋₆alkyl or R⁵⁰; and

R⁵ is halo, —CN, C₁₋₆alkyl, alkynyl, hydroxy, C₁₋₆alkoxy, nitro, —N(R^{a})₂, —C(O)N(R^{a})₂, —CO₂R^{a} or —C(O)R^{a}.

The compound according to Formula I may have a Formula IA

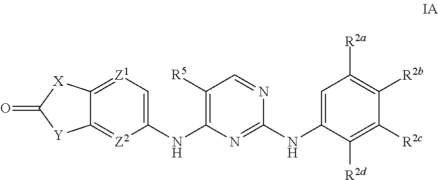

With respect to Formula IA, the variables are as defined for Formula I, and each of R^{2a}, R^{2b}, R^{2c} and R^{2d} is independently for each occurrence as previous defined for R². In some embodiments, X and Y are each independently O or NR¹; each R¹ is H, C₁₋₆alkyl or R⁵⁰; and R⁵ is halo, —CN, C₁₋₆alkyl, nitro, —N(R^{a})₂, —C(O)N(R^{a})₂, —CO₂R^{a} or —C(O)R^{a}. In certain embodiments, one of X and Y is O and the other is NR¹.

In some embodiments of Formula IA, the compound has a Formula IA1 or IA2

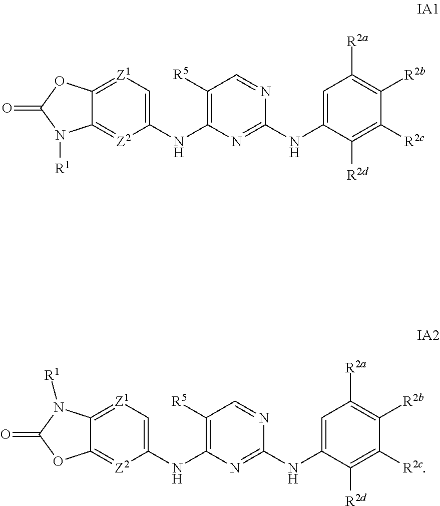

And in some embodiments of Formulas IA, IA1 or IA2, R^{2d} is H; R⁵ is halo or C₁₋₆alkyl; Z¹ is CH, C-halo or C—C₁₋₆alkyl; and Z² is CH.

Another embodiment is a compound of structural formulae IA1 or IA2 where R⁵ is F or CH₃. In a more specific embodiment, each of R^{2a}, R^{2b} and R^{2c} is independently for each occurrence C₁₋₆alkyl, —OR^{a}, —OCF₃, —SR^{a}, —N(R^{c})₂, halo, —OCF₂H, —OCH₂F, —CF₃, —CN, —S(O)₂N(R^{c})₂, —S(O)₂R^{a}, —C(O)R^{a}, —CO₂R^{a}, —C(O)N(R^{c})₂, —(C(R^{a})₂)ₘ—R^{b}, —N(R^{a})—S(O)₂R^{a} or —[N(R^{a})C(O)]ₙR^{a}. In another more specific embodiment, R^{2a}, R^{2b} and R^{2c} are each independently C₁₋₆alkyl, —OR^{a}, —OCF₃, halo, —CF₃ or —CN. In one embodiment, R^{2a} is CH₃; R^{2b} is halo; and R^{2c} is CH₃. In another embodiment, R^{2a} is CH₃; R^{2b} is CH₃; and R^{2c} is halo. In another embodiment, R^{2a} is CH₃; R^{2b} is CH₃; and R^{2c} is CH₃. In a more specific embodiment, R^{2a} is CH₃; R^{2b} is CH₃ and R^{2c} is CH₃, R⁵ is CH₃.

Another embodiment is a compound of structural formulae IA1, where R⁵ is CH₃, and each of R^{2a}, R^{2b} and R^{2c} is independently for each occurrence C₁₋₆alkyl, haloC₁₋₆alkyl, —OR^{a}, —OCF₃, —SR^{a}, —N(R^{c})₂, halo, —OCF₂H, —OCH₂F, —CN, —S(O)₂N(R^{a})₂, —S(O)₂R^{a}, —C(O)R^{a}, —CO₂R^{a}, —C(O)N(R^{c})₂, —(C(R^{a})₂)ₘ—R^{b}, —N(R^{a})—S(O)₂R^{a} or —[N(R^{a})C(O)]ₙR^{a}. In another more specific embodiment, R^{2a}, R^{2b} and R^{2c} are each independently C₁₋₆alkyl, —OR^{a}, —OCF₃, halo, —CF₃ or —CN. In one embodiment, R^{e}a iₛ CH₃; R^{2b} is halo; and R²o iₛ CH₃. In another embodiment, R^{2a} is CH₃; R^{2b} is CH₃; and R^{2c} is halo. In another embodiment, each of R^{2a}, R^{2b} and R^{2c} is independently for each occurrence C₁₋₆alkyl or haloC₁₋₆alkyl. In another embodiment, each of R^{2a}, R^{2b} and R^{2c} is independently for each occurrence C₁₋₆alkyl. In a more specific embodiment, R^{2a} is CH₃; R^{2b} is CH₃ and R^{2c} is CH₃.

Another embodiment is a compound of structural formulae IA1 or IA2, where R^{2b} is H; R⁵ is F or CH₃. In a more specific embodiment, each of R^{e}a and R²o iₛ independently for each occurrence H, C₁₋₆alkyl, —OR^{a}, —OCF₃, —SR^{a}, —N(R^{c})₂, halo, —OCF₂H, —OCH₂F, —CF₃, —CN, —S(O)₂N(R^{c})₂, —S(O)₂R^{a}, —C(O)R^{a}, —CO₂R^{a}, —C(O)N(R^{c})₂, —N(R^{a})—S(O)₂R^{a}, —C(R^{a})₂—N(R^{c})₂ or —[N(R^{a})C(O)]ₙR^{a}; and one of R^{2a} and R^{2c} is not H. In another embodiment, each of R^{2a} and R^{2c} is independently for each occurrence H, C₁₋₆alkyl, —OR^{a}, —OCF₃, halo, —CF₃, —C(R^{a})₂—N(R^{c})₂ or —CN. In another embodiment, R^{2a} is —CF₃ or —CH₃; and R^{2c} is halo or —CH₃. In another embodiment, R^{2a} is H, —CH₃, —CF₃, —OR^{a} or —OCF₃; and R^{2c} is —C(R^{a})₂—N(R^{c})₂.

Another embodiment is a compound of structural formula IA1 or IA2, where R^{2c} is H; and R⁵ is F or CH₃. In one specific embodiment each of R^{2a} and R^{2b} is H, C₁₋₆alkyl, —OR^{a}, —OCF₂H, —OCH₂F, —OCF₃, —SR^{a}, —N(R^{a})₂, halo, —CF₃, —CN, —S(O)₂N(R^{a})₂, —S(O)₂R^{a}, —C(O)R^{a}, —CO₂R^{a}, —C(O)N(R^{a})₂, —N(R^{a})—S(O)₂R^{a}, —C(R^{a})₂—N(R^{c})₂or —[N(R^{a})C(O)]ₙR^{a}; and one of R^{2a} and R^{2b} is not H. In another embodiment, each of R^{2a} and R^{2b} is H, C₁₋₆alkyl, —OR^{a}, —OCF₃, halo, —N(R^{c})₂, —CF₃, —C(R^{a})₂—N(R^{c})₂ or —CN. In another embodiment, R^{2b} is —CF₃ or —CH₃; and R^{2a} is halo or —CH₃. In another embodiment, R^{2a} is H, —CH₃, —CF₃, —OR^{a} or —OCF₃; and R^{2b} is —N(R^{c})₂ or —C(R^{a})₂—N(R^{c})₂. In yet another embodiment, R^{2a} is —N(R^{c})₂ or —C(R^{a})₂—N(R^{c})₂; and R^{2b} is H, —CH₃, —CF₃, —OR^{a} or —OCF₃.

Still another embodiment is a compound of structural formulae IA1 or IA2, where R^{2c} and R^{2d} are H, and R⁵ is F or CH₃; R^{2a} and R^{2b} are taken together with the carbons to which they are attached to form a 4-10 membered partially or fully saturated mono or bicyclic ring that is fused to the phenyl ring, and optionally contains one or more heteroatoms and is optionally substituted with one or more R^{a} and/or R^{b}. To further aide in describing such a fused ring system, examples of the fused rings are, disregarding the unit of unsaturation between the two phenyl ring atoms for simplicity in nomenclature only, cyclopentane, pyrrolidine, imidazolidine, 1,3-dioxolane, oxazolidine, tetrahydrofuran, cyclohexane, morpholine, piperidine, dioxane, oxathiazinane, piperazine, cycloheptane, cycloheptene, azepane, tetrahydroazepine, diazepam, cyclooctane, cyclooctene, azocane, hexahydroazocine, diazocane or hexahydrodiazocine. That is, for example, if the fused ring formed by R^{2a} and R^{2b} is described as “cyclohexane,” then the compound according to formula IA1 would be

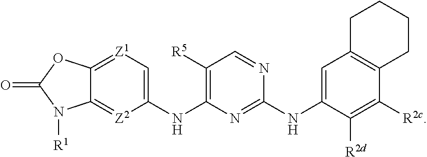

In one embodiment, the fused ring is a 5 membered ring, and in a more specific embodiment the 5 membered ring is cyclopentane, pyrrolidine, imidazolidine, 1,3-dioxolane, oxazolidine or tetrahydrofuran; optionally substituted with one or more R^{a} and/or R^{b}. In a specific embodiment, the 5 membered ring is pyrrolidine, and in an even more specific embodiment the compounds are according to formula IA3:

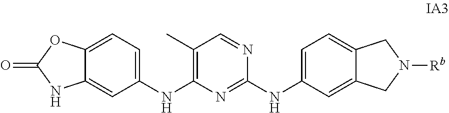

where R^{b} is OH, C₁₋₆alkyl, —CO₂C₁₋₆alkyl, —C(O)C₁₋₆alkyl or —S(O)₂C₁₋₆alkyl. In another embodiment, R^{2a} and R^{2b} are taken together with the carbons to which they are attached to form a 6, 7 or 8 membered partially or fully saturated monocyclic ring, optionally containing one or more heteroatoms and optionally substituted with one or more R^{a} and/or R^{b}. In one embodiment, when the ring is 6 membered, the ring is cyclohexane, morpholine, piperidine, dioxane, oxathiazinane or piperazine; optionally substituted with one or more R^{a} and/or R^{b}. In another embodiment, when the ring is 7 membered, the ring is cycloheptane, cycloheptene, azepane, tetrahydroazepine or diazepam; optionally substituted with one or more R^{a} and/or R^{b}. In yet another embodiment, when the ring is 8 membered, the ring is cyclooctane, cyclooctene, azocane, hexahydroazocine, diazocane or hexahydrodiazocine; optionally substituted with one or more R^{a} and/or R^{b}. For each of the above embodiments, where R^{2a} and R^{2b} are taken together with the carbons to which they are attached to form a 5, 6, 7 or 8 membered partially or fully saturated monocyclic ring, there is a more specific embodiment where there are 0, 1, 2 or 3 each of R^{a} and R^{b}, and R^{a} is C₁₋₆alkyl; and each R^{b} is independently for each occurrence ═O, —OR^{a}, haloC₁₋₃alkyloxy, —SR^{a}, —N(R^{c})₂, halo, —CF₃, —CN, —S(O)₂N(R^{c})₂, —S(O)₂R^{a}, —C(O)R^{a}, —CO₂R^{a}, —C(O)N(R^{c})₂, —N(R^{a})—S(O)₂R^{a} or —C(R^{a})₂—N(R^{a})₂. For example, in one embodiment, there is at least one R^{b} that is ═O; and optionally an R^{a} that is optionally substituted C₁₋₆alkyl.

Alternatively, the compound may have a Formula IB

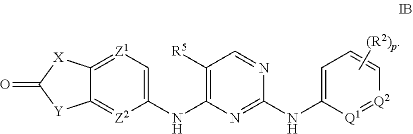

With respect to Formula IB, Q¹ and Q² are each independently N or CH provided at least one of Q¹ and Q² is N. In some embodiments, X and Y are each independently O or NR¹; each R¹ is independently for each occurrence H, C₁₋₆alkyl or R⁵⁰; p is 0, 1, 2 or 3; and R⁵ is halo, —CN, C₁₋₆alkyl, nitro, —N(R^{a})₂, —C(O)N(R^{a})₂, —CO₂R^{a} or —C(O)R^{a}.

In some embodiments of Formula IB, the compound has a Formula IB1, IB2 or IB3

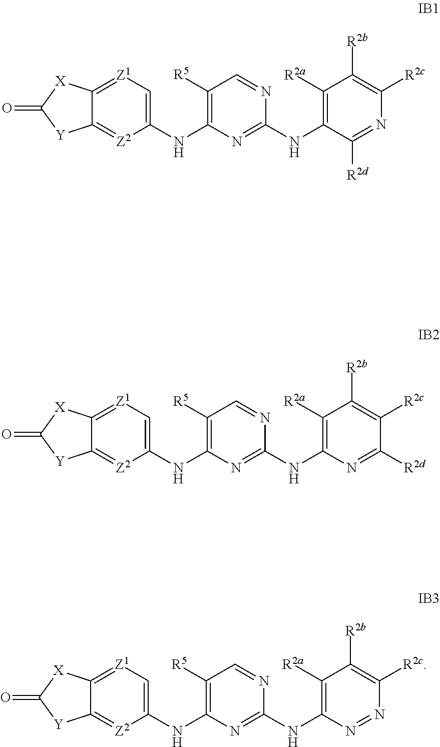

With respect to Formulas IB1, IB2 and IB3, each of R^{2a}, R^{2b}, R^{2c} and R^{2d}, if present, is independently for each occurrence as defined for R².

One embodiment is a compound of structural formula IB1 or IB2, where X and Y are each independently NR¹. In a more specific embodiment, X and Y are each independently NH or NC₁₋₆alkyl. In an even more specific embodiment, X and Y are each independently NH or NCH₃. In one embodiment, where X and Y are defined more specifically as mentioned, R⁵ is halo or C₁₋₆alkyl; Z¹ is CH, C-Halo or C—C₁₋₆alkyl; and Z² is CH. In another embodiment, R^{2a} and R^{2d} are H; and R⁵ is F or CH₃. In another embodiment, each of R^{2b} and R^{2c} is independently for each occurrence H, C₁₋₆alkyl, —OR^{a}, —OCH₂F, —OCF₃, —SR^{a}, —N(R^{c})₂, halo, —OCF₂H, —CF₃, —CN, —S(O)₂N(R^{c})₂, —S(O)₂R^{a}, —C(O)R^{a}, —CO₂R^{a}, —C(O)N(R^{c})₂, —N(R^{a}) —S(O)₂R^{a}, —C(R^{a})₂—N(R^{c})_{2 or —[N(R}^{a})C(O)]ₙR^{a}; and one of R^{2b} and R^{2c} is not H. In another such embodiment, each of R^{2b} and R^{2c} is independently for each occurrence H, C₁₋₆alkyl, —N(R^{c})₂, halo, —CF₃, —CN, —S(O)₂N(R^{a})₂, —S(O)₂R^{a}, —C(O)R^{a}, —CO₂R^{a}, —C(O)N(R^{c})₂, —C(R^{a})₂—N(R^{c})_{2 or —N(R}^{a})—S(O)₂R^{a}. In another embodiment, R^{2b} is H, halo, —CF₃, —CN or —CH₃; and R^{2c} is —N(R^{c})₂, —S(O)₂N(R^{c})₂, —S(O)₂R^{a}, —C(O)N(R^{c})₂ or —C(R^{a})₂—N(R^{c})₂.

Another embodiment is a compound of structural formula IB1 or IB2, where X is O and Y is NR¹. In one embodiment, R⁵ is halo or C₁₋₆alkyl; Z¹ is CH, C-Halo or C—C₁₋₆alkyl; and Z² is CH. In another embodiment, R^{2a} and R^{2d} are H; and R⁵ is F or CH₃. In a more specific embodiment, each of R^{2b} and R^{2c} is independently for each occurrence H, C₁₋₆alkyl, —OR^{a}, —OCF₃, —SR^{a}, —N(R^{c})₂, halo, —OCF₂H, —OCH₂F, —CF₃, —CN, —S(O)₂N(R^{c})₂, —S(O)₂R^{a}, —C(O)R^{a}, —CO₂R^{a}, —C(O)N(R^{c})₂, —N(R^{c})—S(O)₂R^{a}, —C(R^{a})₂—N(R^{c})₂ or —[N(R^{a})C(O)]ₙR^{a}; and one of R^{2b} and R^{2c} is not H. In another embodiment, each of R^{2b} and R^{2c} is independently for each occurrence H, C₁₋₆alkyl, —N(R^{c})₂, halo, —CF₃, —CN, —S(O)₂N(R^{c})₂, —S(O)₂R^{a}, —C(O)R^{a}, —CO₂R^{a}, —C(O)N(R^{c})₂, —C(R^{a})₂—N(R^{c})₂ or —N(R^{a})—S(O)₂R^{a}. In a more specific embodiment R^{2b} is H, halo, —CF₃, —CN or —CH₃; and R^{2c} is —N(R^{c})₂, —S(O)₂N(R^{a})₂, —S(O)₂R^{a}, —C(O)N(R^{c})₂ or —C(R^{c})₂—N(R^{c})₂. In another embodiment, R^{2b} is H, halo, —CF₃, —CN or —CH₃; and R^{2c} is —N(R^{c})₂ or —C(R^{a})₂—N(R^{c})₂.

Another embodiment is a compound of structural formula IB1 or IB2, where X is O; Y is NR¹; Z¹ is CH, C-Halo or C—C₁₋₆alkyl; Z² is CH; R^{2a} and R^{2d} are H; and R⁵ is F or CH₃, R^{2b} and R^{2c} are taken together with the carbons to which they are attached to form a 4-10 membered partially or fully saturated mono or bicyclic ring, optionally containing one or more heteroatoms and optionally substituted with one or more R^{a} and/or R^{b}. In one embodiment, the ring is a 5, 6, 7 or 8 membered partially or fully saturated monocyclic ring optionally substituted with one or more R^{a} and/or R^{b}. In one embodiment, the 5, 6, 7 or 8 membered partially or fully saturated monocyclic ring is cyclopentane, pyrrolidine, imidazolidine, 1,3-dioxolane, oxazolidine, tetrahydrofuran, cyclohexane, morpholine, piperidine, dioxane, oxathiazinane, piperazine, cycloheptane, cycloheptene, azepane, tetrahydroazepine, diazepam, cyclooctane, cyclooctene, azocane, hexahydroazocine, diazocane or hexahydrodiazocine; optionally substituted with one or more R^{a} and/or R^{b}. For each of the above embodiments, where R^{2b} and R^{2c} are taken together with the carbons to which they are attached to form a 5, 6, 7 or 8 membered partially or fully saturated monocyclic ring, there is a more specific embodiment where there are 0, 1, 2 or 3 each of R^{a} and R^{b}, and R^{a} is C₁₋₆alkyl; and each R^{b} is independently for each occurrence ═O, —OR^{a}, haloC₁₋₃alkyloxy, —SR^{a}, —N(R^{c})₂, halo, —CF₃, —CN, —S(O)₂N(R^{c})₂, —S(O)₂R^{a}, —C(O)R^{a}, —CO₂R^{a}, —C(O)N(R^{c})₂, —N(R^{a})—S(O)₂R^{a} or —C(R^{a})₂—N(R^{c})₂. For example, in one embodiment, there is at least one R^{b} that is ═O; and optionally an R^{a} that is C₁₋₆alkyl. In this embodiment compounds such as IV-45, IV-46 and IV-47 are encompassed.

One embodiment is a compound of structural formula IB3, where X is O and Y is NR¹. In one embodiment, R⁵ is halo or C₁₋₆alkyl; Z¹ is CH, C-Halo or C—C₁₋₆alkyl; and Z² is CH. In another embodiment, R^{2a} is H; and R⁵ is F or CH₃. In a more specific embodiment, each of R^{2b} and R^{2c} is independently for each occurrence H, C₁₋₆alkyl, —OR^{a}, —OCF₃, —SR^{a}, —N(R^{c})₂, halo, —OCF₂H, —OCH₂F, —CF₃, —CN, —S(O)₂N(R^{c})₂, —S(O)₂R^{a}, —C(O)R^{a}, —CO₂R^{a}, —C(O)N(R^{c})₂, —N(R^{a})—S(O)₂R^{a}, —C(R^{a})₂—N(R^{c})₂ or —[N(R^{a})C(O)]ₙR^{a}; and one of R^{2b} and R^{2c} is not H. In another embodiment, each of R^{2b} and R^{2c} is independently for each occurrence H, C₁₋₆alkyl, —N(R^{c})₂, halo, —CF₃, —CN, —S(O)₂N(R^{c})₂, —S(O)₂R^{a}, —C(O)R^{a}, —CO₂R^{a}, —C(O)N(R^{c})₂, —C(R^{a})₂—N(R^{c})₂ or —N(R^{a})—S(O)₂R^{a}. In a more specific embodiment R^{2b} is H, halo, —CF₃, —CN or —CH₃; and R^{2c} is —N(R^{c})₂, —S(O)₂N(R^{c})₂, —S(O)₂R^{a}, —C(O)N(R^{c})₂ or —C(R^{a})₂—N(R^{c})₂. In another embodiment, R^{2b} is H, halo, —CF₃, —CN or —CH₃; and R^{2c} is —N(R^{c})₂ or —C(R^{a})₂—N(R^{c})₂.

Another embodiment is a compound of structural formula IB3, where X is O; Y is NR¹; Z¹ is CH, C-Halo or C—C₁₋₆alkyl; Z² is CH; R^{2c} and R^{2d} are H; and R⁵ is F or CH₃, R^{2b} and R^{2c} are taken together with the carbons to which they are attached to form a 4-10 membered partially or fully saturated mono or bicyclic ring, optionally containing one or more heteroatoms and optionally substituted with one or more R^{a} and/or R^{b}. In one embodiment, the ring is a 5, 6, 7 or 8 membered partially or fully saturated monocyclic ring optionally substituted with one or more R^{a} and/or R^{b}. In one embodiment, the 5, 6, 7 or 8 membered partially or fully saturated monocyclic ring is cyclopentane, pyrrolidine, imidazolidine, 1,3-dioxolane, oxazolidine, tetrahydrofuran, cyclohexane, morpholine, piperidine, dioxane, oxathiazinane, piperazine, cycloheptane, cycloheptene, azepane, tetrahydroazepine, diazepam, cyclooctane, cyclooctene, azocane, hexahydroazocine, diazocane or hexahydrodiazocine; optionally substituted with one or more R^{a} and/or R^{b}. For each of the above embodiments, where R^{2b} and R^{2c} are taken together with the carbons to which they are attached to form a 5, 6, 7 or 8 membered partially or fully saturated monocyclic ring, there is a more specific embodiment where there are 0, 1, 2 or 3 each of R^{a} and R^{b}, and R^{a} is C₁₋₆alkyl; and each R^{b} is independently for each occurrence ═O, —OR^{a}, haloC₁₋₃alkyloxy, —SR^{a}, —N(R^{c})₂, halo, —CF₃, —CN, —S(O)₂N(R^{a})₂, —S(O)₂R^{a}, —C(O)R^{a}, —CO₂R^{a}, —C(O)N(R^{c})₂, —N(R^{a})—S(O)₂R^{a} or —C(R^{a})₂—N(R^{c})₂.

Another embodiment of the compounds of structural formula I, is a compound according to formula II:

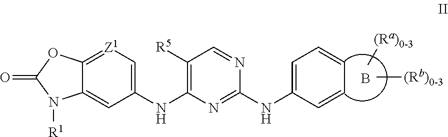

where the variables are defined in the same way as for the those of formula I, and further: two of R² combine to form ring B; ring B, together with the two phenyl ring atoms to which it is attached, forms a 5, 6 or 7-membered ring, optionally containing 1, 2 or 3 heteroatoms independently selected from N(R^{c}), O and S; each R^{a} is C₁₋₆alkyl; and each R^{b} is independently for each occurrence ═O, —OR^{a}, haloC₁₋₃alkyloxy, —SR^{a}, —N(R^{c})₂, halo, —CF₃, —CN, —S(O)₂N(R^{c})₂, —S(O)₂R^{a}, —C(O)R^{a}, —CO₂R^{a}, —C(O)N(R^{c})₂, —N(R^{a})—S(O)₂R^{a} or —C(R^{a})₂—N(R^{c})₂. In one more specific embodiment, Z¹ is CH, C-halo or C—C₁₋₆alkyl. To further aide in describing ring B, examples of B rings are, disregarding the unit of unsaturation between the two phenyl ring atoms for simplicity in nomenclature only, cyclopentane, pyrrolidine, imidazolidine, 1,3-dioxolane, oxazolidine, tetrahydrofuran, cyclohexane, morpholine, piperidine, dioxane, oxathiazinane, piperazine, cycloheptane, cycloheptene, azepane, tetrahydroazepine, diazepam, cyclooctane, cyclooctene, azocane, hexahydroazocine, diazocane or hexahydrodiazocine. That is, for example, if ring B is described as “cyclohexane,” then the compound would be according to the formula

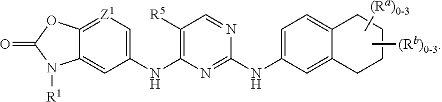

Another embodiment is a compound according to formula IA1, where R^{2.3d} is H; R⁵ is halo or C₁₋₆alkyl; Z¹ is CH, C-halo or C—C₁₋₆alkyl; Z² is CH; and each of R^{2a}, R^{2b} and R^{2c} is independently for each occurrence C₁₋₆alkyl, —OR^{a}, —OCF₃, —SR^{a}, —N(R^{c})₂, halo, —OCF₂H, —OCH₂F, —CF₃, —CN, —S(O)₂N(R^{c})₂, —S(O)₂R^{a}, —C(O)R^{a}, —CO₂R^{a}, —C(O)N(R^{c})₂, —(C(R^{a})₂)ₘ—R^{b}, —N(R^{a})—S(O)₂R^{a} or —[N(R^{a})C(O)]ₙR^{a}, provided one of R^{2a}, R^{2b} and R^{2c} is —N(R^{c})₂, —C(O)N(R^{c})₂ or —(C(R^{a})₂)ₘ—R^{b}. In a more specific embodiment, R⁵ is F or CH₃. In another embodiment, one of R^{2a}, R^{2b} and R^{2c} is —N(R^{a})₂. In another embodiment, one of R^{2a}, R^{2b} _{and R}^{2c} is —(C(R^{a})₂)ₘ—R^{b}. In a more specific embodiment, R⁵ is F or CH₃. In a more specific embodiment, the one of R^{2a}, R^{2b} and R^{2c} that is —N(R^{c})₂, is:

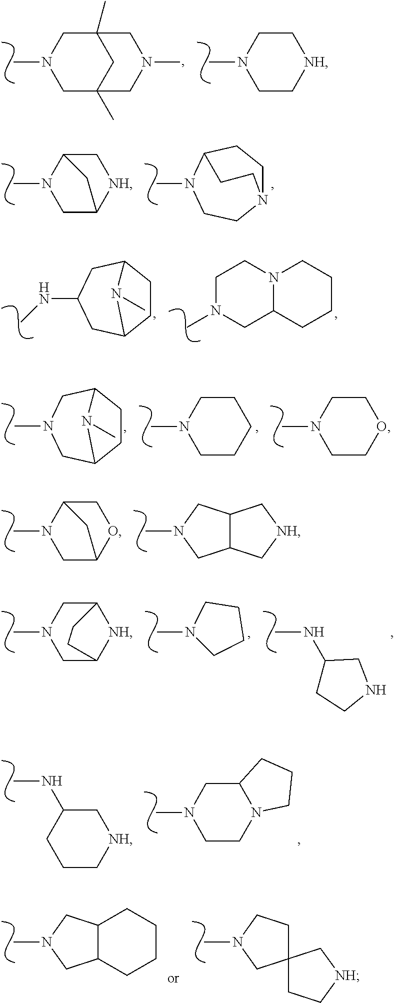

optionally substituted with one or more of the same or different R^{a} and/or R^{b} groups. In a more specific embodiment, the one of R^{2a}, R^{2b} and R^{2c} that is —(C(R^{a})₂)ₘ—R^{b}, is even more specifically —C(R^{a})₂—N(R^{c})₂. In an even more specific embodiment, the one of R^{2a}, R^{2b} _{and R}^{2c} that is —C(R^{a})₂—N(R^{c})₂ is:

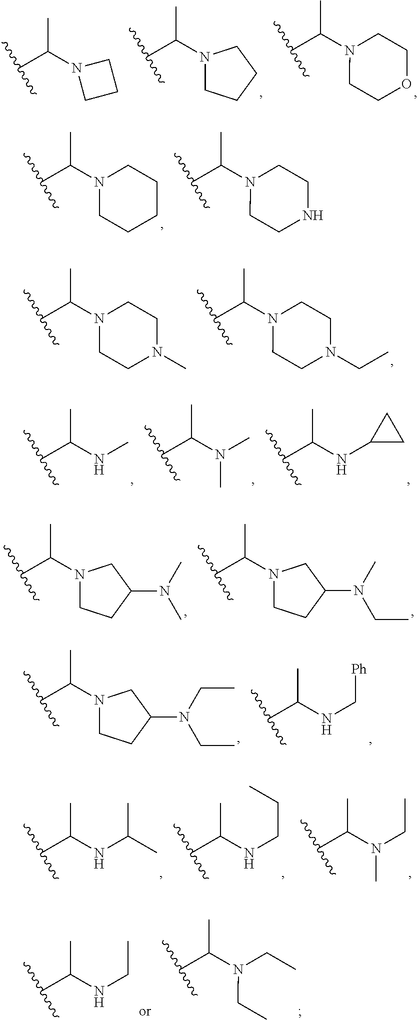

optionally substituted with one or more of the same or different R^{a} and/or R^{b} groups.

In one embodiment, at least one R² group is a water-solubilizing group, that is, a group that has hydrophilic character sufficient to improve or increase the water-solubility of the compound in which it is included, as compared to an analog compound that does not include the group. The hydrophilic character can be achieved by, for example, the inclusion of functional groups that ionize under the conditions of use to form charged moieties (e.g., carboxylic acids, sulfonic acids and salts, phosphoric acids and salts, amines, etc.); groups that include permanent charges (e.g., quaternary ammonium groups); and/or heteroatoms or heteroatomic groups. For example, —O—(C(R^{a})₂)ₘ—R^{b}, —S—(C(R^{a})₂)ₘ—R^{b}, —O—(C(R^{b})₂)ₘ—R^{a}, —N(R^{a})—(C(R^{a})₂)ₘ—R^{b}, —O—(CH₂)ₘ—CH((CH₂)ₘR^{b})R^{b}, —C(O)N(R^{a})—(C(R^{a})₂)ₘ—R^{b} and —N((C(R^{a})₂)ₘR^{b})₂. More specific examples include —O—C₁₋₆alkylene-R^{b}, —S—C₁₋₆alkylene-R^{b}, —O—C₁₋₆alkylene-R^{a} where R^{a} is heterocyclyl, —N(R^{a})—C₁₋₆alkylene-R^{b}, —O—C₁₋₆alkylene—CH((CH₂)₁₋₂R^{b})R^{b}, —C(O)N(R^{a})—C₁₋₆alkylene-R^{b} and —N((C(R^{a})₂)₁₋₃R^{b})₂. Even more specific examples include —O—C₁₋₄alkylene-R^{b}, —S—C₁₋₄alkylene-R^{b}, —O—C₁₋₄alkylene-R^{a} where R^{a} is heterocyclyl, —N(H)—-C₁₋₄alkylene-R^{b}, —O—C₁₋₄alkylene—CH((CH₂)₁₋₂R^{b})R^{b}, —C(O)N(H)—C₁₋₄alkylene-R^{b} and —N((CH₂)₁₋₃R^{b})₂. In another specific example, in accord with the formula given above for water solubilizing groups, the water solubilizing group is an amino acid tethered from the molecule via a bond to the nitrogen of the amino acid. In a more specific example, a water solubilizing group is an α-amino acid or derivative thereof attached to the parent ring, e.g. ring A and/or at Z¹ or Z², via the nitrogen of the a-amino acid, for example —N(H)C(R^{a})₂—R^{b}, where R^{b} is —CO₂R^{a} or —C(O)N(R^{c})₂. In another specific embodiment, the water-solubilizing group is morpholino, piperidinyl, N—C₁₋₆alkyl piperidinyl, piperazinyl, N—C₁₋₆alkyl piperazinyl, pyrrolidinyl, N—C₁₋₆alkyl pyrrolidinyl, diazepinyl, N—C₁₋₆alkyl azepinyl, homopiperazinyl, N—C₁₋₆alkyl homopiperazinyl, imidazoyl, and the like. In another example the water-solubilizing group is one of the aforementioned rings tethered to the parent molecule via an alkylene, alkylidene, alkylidyne linker. In a more specific embodiment, the water-solubilizing group is one of the aforementioned rings tethered to the parent molecule via a C₁₋₆alkylene, where one or two of the alkylene carbons is, independently, replaced with one of O, S or NH, but not where any two of the aforementioned heteroatoms are contiguous in the linker. Other water solubilizing groups are well-known and include, by way of example, hydrophilic groups such as alkyl or heterocycloaliphatic groups substituted with one or more of an amine, alcohol, a carboxylic acid, a phosphorous acid, a sulfoxide, a carbohydrate, a sugar alcohol, an amino acid, a thiol, a polyol, an ether, a thioether, and a quaternary amine salt.

For each of the above embodiments of the compounds of structural formulae I, IA, IA1, IA2, IA3, IB, IB1, IB2, IB3 and II, there is another embodiment where R^{a} is H or R⁵⁰; R⁵⁰ is —CH₂OP(O)(OR¹¹)₂; and each R¹¹ is independently for each occurrence R^{a} or a monovalent cationic group; or two R¹¹, together with the atoms to which they are attached, form a 4-8 membered cyclic phosphate group, or two R¹¹ together represent a divalent cationic group. Also, for each of these embodiments, there is a more specific embodiment where each R¹¹ is independently for each occurrence H, t-butyl, or a pharmaceutically acceptable cation, such as HOCH₂CH₂N(CH₃)₃⁺, Na⁺, Li⁺ or K⁺.

As mentioned, the 2,4-pyrimidinediamine compounds and prodrugs, as well as the salts thereof, can also be in the form of hydrates, solvates, and N-oxides, as is well-known in the art. One embodiment is a pharmaceutically acceptable salt form of a compound of formula I. The pharmaceutically acceptable salts of the present disclosure can be formed by conventional means, such as by reacting the free base form of the product with one or more equivalents of the appropriate acid in a solvent or medium in which the salt is insoluble or in a solvent such as water which is removed in vacuo, by freeze drying, or by exchanging the anions of an existing salt for another anion on a suitable ion exchange resin. The present disclosure contemplates within its scope solvates of the 2,4-pyrimidinediamine compounds and salts and hydrates thereof, for example, a hydrated formate salt.

Exemplary compounds according to Formula I include, but are not limited to, those listed below in List 1.

### List 1: Exemplary Compounds According to Formula I

I-1 N4-(benzo[d]oxazol-2(3H)-on-5-yl)-N2-(3-formylphenyl)-5-methylpyrimidine-2,4-diamine;

I-2 N4-(benzo[d]oxazol-2(3H)-on-5-yl)-N2-(3-aminocarbonylphenyl)-5-methylpyrimidine-2,4-diamine;

I-3 N4-(benzo[d]oxazol-2(3H)-on-5-yl)-N2-(4-aminocarbonylphenyl)-5-methylpyrimidine-2,4-diamine;

I-4 N4-(benzo[d]oxazol-2(3H)-on-5-yl)-N2-(4-formylphenyl)-5-methylpyrimidine-2,4-diamine;

I-5 N4-(benzo[d]oxazol-2(3H)-on-5-yl)-N2-(3-methyl-4-(1,5,7-trimethyl-3,7-diazabicyclo [3.3.1]nonan-3-yl)phenyl)-5-methylpyrimidine-2,4-diamine;

I-6 N4-(benzo[d]oxazol-2(3H)-on-5-yl)-N2-(3-fluoro-4-(1,5,7-trimethyl-3,7-diazabicyclo [3.3.1]nonan-3-yl)phenyl)-5-methylpyrimidine-2,4-diamine;

I-7 N4-(3-n-propylbenzo[d]oxazol-2(3H)-on-5-yl)-N2-((3-methylsulfonyl)phenyl)-5-methylpyrimidine-2,4-diamine;

I-9 N4-(3-isopropylbenzo[d]oxazol-2(3H)-on-5-yl)-N2-((3-methylsulfonyl)phenyl)-5-methylpyrimidine-2,4-diamine;

I-16 N4-(benzo[d]oxazol-2(3H)-on-5-yl)-N2-(3-methylsulfonyl)phenyl)-5-methylpyrimidine-2,4-diamine;

I-17 N4-(benzo[d]oxazol-2(3H)-on-5-yl)-N2-(4-methylsulfonyl)phenyl)-5-methylpyrimidine-2,4-diamine;

I-20 N4-(benzo[d]oxazol-2(3H)-on-5-yl)-N2-(3-methylsulfonyl)phenyl)-5-fluoropyrimidine-2,4-diamine;

I-21 N4-(benzo[d]oxazol-2(3H)-on-5-yl)-N2-(4-methylsulfonyl)phenyl)-5-fluoropyrimidine-2,4-diamine;

I-22 N4-(benzimidazolin-2-on-5-yl)-N2-(3-methylsulfonyl)phenyl-5-methylpyrimidine-2,4-diamine;

I-23 N4-(benzimidazolin-2-on-5-yl)-N2-(4-methylsulfonyl)phenyl-5-fluoropyrimidine-2,4-diamine;

I-26 N4-(benzo[d]oxazol-2(3H)-on-5-yl)-N2-(3-cyanophenyl)-5-methylpyrimidine-2,4-diamine;

I-27 N4-(benzo[d]oxazol-2(3H)-on-5-yl)-N2-(4-cyanophenyl)-5-methylpyrimidine-2,4-diamine;

I-28 N4-(benzimidazolin-2-on-5-yl)-N2-(3-cyanophenyl)-5-methylpyrimidine-2,4-diamine;

I-29 N4-(benzimidazolin-2-on-5-yl)-N2-(4-cyanophenyl)-5-methylpyrimidine-2,4-diamine;

I-33 N4-(3-phosphorylmethylbenzo[d]oxazol-2(3H)-on-5-yl)-N2-((3-methylsulfonyl)phenyl)-5-methylpyrimidine-2,4-diamine;

I-36 N4-(1,3-dimethylbenzimidazolin-2-on-5-yl)-N2-((3-methylsulfonyl)phenyl)-5-methylpyrimidine-2,4-diamine;

I-41 N4-(1,3-dimethylbenzimidazolin-2-on-5-yl)-N2-((3-methylsulfonyl)phenyl)-5-fluoropyrimidine-2,4-diamine;

I-44 N4-(benzo[d]oxazol-2(3H)-on-5-yl)-N2-(3-cyanophenyl)-5-fluoropyrimidine-2,4-diamine;

I-45 N4-(benzo[d]oxazol-2(3H)-on-5-yl)-N2-(4-cyanophenyl)-5-fluoropyrimidine-2,4-diamine;

I-46 N4-(benzo[d]oxazol-2(3H)-on-5-yl)-N2-((3-morpholinyl)phenyl)-5-methylpyrimidine-2,4-diamine;

I-47 N4-(benzo[d]oxazol-2(3H)-on-5-yl)-N2-((3-morpholinyl)phenyl)-5-fluoropyrimidine-2,4-diamine;

I-48 N4-(benzo[d]oxazol-2(3H)-on-6-yl)-N2-((3-morpholinyl)phenyl)-5-methylpyrimidine-2,4-diamine;

I-49 N4-(3-methylbenzo[d]oxazol-2(3H)-on-6-yl)-N2-((3-morpholinyl)phenyl)-5-methylpyrimidine-2,4-diamine;

I-59 N2-((3-methylsulfonyl)phenyl)-N4-(3-methyl-2-oxo-2,3-dihydrobenzo[d]oxazol-6-yl)-5-fluoropyrimidine-2,4-diamine;

I-60 N2-(4-methylsulfonyl)phenyl)-N4-(3-methyl-2-oxo-2,3-dihydrobenzo[d]oxazol-6-yl)-5-fluoropyrimidine-2,4-diamine;

I-65 N2-((3-methylsulfonyl)phenyl)-N4-(3-methyl-2-oxo-2,3-dihydrobenzo[d]oxazol-6-yl)-5-methylpyrimidine-2,4-diamine;

I-66 N2-((4-methylsulfonyl)phenyl)-N4-(3-methyl-2-oxo-2,3-dihydrobenzo[d]oxazol-6-yl)-5-methylpyrimidine-2,4-diamine;

I-69 N2-((3-methylsulfonyl)phenyl)-N4-(3-methyl-2-oxo-2,3-dihydrobenzo[d]oxazol-5-yl)-5-methylpyrimidine-2,4-diamine;

I-70 N2-((4-methylsulfonyl)phenyl)-N4-(3-methyl-2-oxo-2,3-dihydrobenzo[d]oxazol-5-yl)-5-methylpyrimidine-2,4-diamine;

I-77 N2-((3-methyl sulfonyl)phenyl)-N4-(3-methyl-2-oxo-2,3-dihydrobenzo[d]oxazol-5-yl)-5-fluoropyrimidine-2,4-diamine;

I-78 N2-((4-methylsulfonyl)phenyl)-N4-(3-methyl-2-oxo-2,3-dihydrobenzo[d]oxazol-5-yl)-5-fluoropyrimidine-2,4-diamine;

I-100 N2-((3-methyl sulfonyl)phenyl)-N4-(2-oxo-2,3-dihydrobenzo[d]oxazol-6-yl)-5-methylpyrimidine-2,4-diamine;

I-101 N2-((4-methylsulfonyl)phenyl)-N4-(2-oxo-2,3-dihydrobenzoxazol-6-yl)-5-methylpyrimidine-2,4- diamine;

I-106 N4-(benzoxazolin-2-on-5-yl)-N2-(3-trifluoromethoxyphenyl)-5-methylpyrimidine-2,4-diaminetrifluoroacetate salt;

I-107 N4-(benzoxazolin-2-on-5-yl)-N2-(3-trifluoromethoxyphenyl)-5-fluoropyrimidine-2,4-diamine trifluoroacetate salt;

I-108 N4-(benzoxazolin-2-on-5-yl)-N2-(4-trifluoromethoxyphenyl)-5-methylpyrimidine-2,4-diamine trifluoroacetate salt;

I-109 N4-(benzoxazolin-2-on-5-yl)-N2-(4-trifluoromethoxyphenyl)-5-fluoropyrimidine-2,4-diaminetrifluoroacetate salt;

I-110 N4-(benzoxazolin-2-on-5-yl)-N2-[3-trifluoromethyl-4-(4-ethylpiperazin-1-yl)phenyl]-5-methylpyrimidine-2,4-diamine;

I-111 N4-(benzoxazolin-2-on-5-yl)-N2-[3-methyl-4-(4-methylpiperazin-1-yl)phenyl]-5-methylpyrimidine-2,4-diamine;

I-115 N4-(benzoxazolin-2-on-5-yl)-N2-(3,4,5-trimethoxyphenyl)-5-fluoropyrimidine-2,4-diamine;

I-116 N2-(3-(difluoromethoxy)-4-methoxyphenyl)-N4-(benzo[d]oxazol-2 (3H)-on-5-yl)-5-methylpyrimidine-2,4-diamine;

I-117 N4-(benzo[d]oxazol-2 (3H)-on-5-yl)-N2-(4-trifluoromethyl sulfonyl)phenyl-5-methylpyrimidine-2,4-diamine;

I-118 N4-(benzo[d]oxazol-2(3H)-on-5-yl)-N2-(3-trifluoromethylsulfonyl)phenyl-5-methylpyrimidine-2,4-diamine;

I-119 N4-(benzo[d]oxazol-2(3H)-on-5-yl)-N2-(3,4,5-trimethoxy)phenyl-5-methylpyrimidine-2,4-diamine;

I-120 N4-(benzo[d]oxazol-2(3H)-on-5-yl)-N2-((4-(1,4-diazabicyclo[3.2.2]nonan-4-yl)-3-methyl)phenyl)-5-methylpyrimidine-2,4-diamine;

I-121 N4-(benzo[d]oxazol-2(3H)-on-5-yl)-N2-(4-(8-methyl-8-azabicyclo[3.2.1]octan-3-ylamino)phenyl)-5-methylpyrimidine-2,4-diamine;

I-122 N4-(benzo[d]oxazol-2(3H)-on-5-yl)-N2-((4-(dihydro-1H-pyrido [1,2-a]pyrazin-2 (6H,7H, 8H,9H,9aH)-yl)-3-methyl)phenyl)-5-methylpyrimidine-2,4-diamine;

I-123 N4-(benzo[d]oxazol-2(3H)-on-5-yl)-N2-(4-(8-methyl-2,8-diazabicyclo[3.2.1]octan-2-yl)phenyl)-5-methylpyrimidine-2,4-diamine;

I-124 N4-(benzo[d]oxazolin-2(3H)-one-5-yl)-5-methyl-N2-[3-(morpholin-4-yl)-4-trifluoromethoxyphenyl]-2,4-pyrimidinediamine;

I-125 N4-(benzo[d]oxazolin-2(3H)-on-5-yl)-N2-[3-trifluoromethyl-2-(4-methylpiperazin-1-yl)pyridin-5-yl]-5-methylpyrimidine-2,4-diamine;

I-126 4-[5-Methyl-4-(2-oxo-2,3-dihydro-benzooxazol-5-ylamino)-pyrimidin-2-ylamino]-benzoic acid;

I-127 N-(2-Diethylamino-ethyl)-4-[5-methyl-4-(2-oxo-2,3-dihydro-benzooxazol-5-ylamino)-pyrimidin-2-ylamino]-benzamide;

I-128 5-{2-[4-(3-Diethylamino-pyrrolidine-1-carbonyl)-phenylamino]-5-methyl-pyrimidin-4-ylamino}-3H-benzooxazol-2-one;

I-129 5-[2-(4-Acetyl-phenylamino)-5-methyl-pyrimidin-4-ylamino]-3H-benzooxazol-2-one;

I-130 5-[2-(3-Acetyl-phenylamino)-5-methyl-pyrimidin-4-ylamino]-3H-benzooxazol-2-one;

I-131 2-Methyl-5-[5-methyl-4-(2-oxo-2,3-dihydro-benzooxazol-5-ylamino)-pyrimidin-2-ylamino]-benzonitrile;

I-132 N,N-Dimethyl-4-[5-methyl-4-(2-oxo-2,3-dihydro-benzooxazol-5-ylamino)-pyrimidin-2-ylamino]-benzamide;

I-133 N-Methyl-4-[5-methyl-4-(2-oxo-2,3-dihydro-benzooxazol-5-ylamino)-pyrimidin-2-ylamino]-benzamide;

I-134 N-Cyclopropyl-4-[5-methyl-4-(2-oxo-2,3-dihydro-benzooxazol-5-ylamino)-pyrimidin-2-ylamino]-benzamide formate salt

I-135 4-[5-Methyl-4-(2-oxo-2,3-dihydro-benzooxazol-5-ylamino)-pyrimidin-2-ylamino]-N-phenyl-benzamide;

I-136 4-[5-Methyl-4-(2-oxo-2,3-dihydro-benzooxazol-5-ylamino)-pyrimidin-2-ylamino]-2-pyrrolidin-1-yl-benzamide;

I-137 N-Ethyl-4-[5-methyl-4-(2-oxo-2,3-dihydro-benzooxazol-5-ylamino)-pyrimidin-2-ylamino]-benzamide;

I-138 N-Cyclobutyl-4-[5-methyl-4-(2-oxo-2,3-dihydro-benzooxazol-5-ylamino)-pyrimidin-2-ylamino]-benzamide formic acid salt;

I-139 N-Isopropyl-4-[5-methyl-4-(2-oxo-2,3-dihydro-benzooxazol-5-ylamino)-pyrimidin-2-ylamino]-benzamide;

I-140 N-Cyclopropyl-4-[5-methyl-4-(2-oxo-2,3-dihydro-benzooxazol-5-ylamino)-pyrimidin-2-ylamino]-benzamide formate salt;

I-141 2-Chloro-4-[5-methyl-4-(2-oxo-2,3-dihydro-benzooxazol-5-ylamino)-pyrimidin-2-ylamino]-benzamide;

I-142 N-Cyclopropyl-4-[5-methyl-4-(2-oxo-2,3-dihydro-benzooxazol-5-ylamino)-pyrimidin-2-ylamino]-benzamide trifluoroacetic acid salt;

I-143 N-Cyclopropyl-4-[5-methyl-4-(2-oxo-2,3-dihydro-benzooxazol-5-ylamino)-pyrimidin-2-ylamino]-benzamide;

I-144 N-Cyclobutyl-4-[5-methyl-4-(2-oxo-2,3-dihydro-benzooxazol-5-ylamino)-pyrimidin-2-ylamino]-benzamide;

I-145 4-[5-Methyl-4-(2-oxo-3-propionyl-2,3-dihydro-benzooxazol-5-ylamino)-pyrimidin-2-ylamino]-benzamide;

I-146 di-tert-butyl (5-(2-(4-carbamoylphenylamino)-5-methylpyrimidin-4-ylamino)-2-oxobenzo[d]oxazol-3(2H)-yl)methyl phosphate;

I-147 (5-(2-(4-carbamoylphenylamino)-5-methylpyrimidin-4-ylamino)-2-oxobenzo[d]oxazol-3(2H)-yl)methyl dihydrogen phosphate;

I-148 sodium (5-(2-(4-carbamoylphenylamino)-5-methylpyrimidin-4-ylamino)-2-oxobenzo[d]oxazol-3(2H)-yl)methyl phosphate;

I-150 (5-(2-(4-(cyclobutylcarbamoyl)phenylamino)-5-methylpyrimidin-4-ylamino)-2-oxobenzo[d]oxazol-3(2H)-yl)methyl dihydrogen phosphate;

I-151 sodium (5-(2-(4-(cyclobutylcarbamoyl)phenylamino)-5-methylpyrimidin-4-ylamino)-2-oxobenzo[d]oxazol-3(2H)-yl)methyl phosphate;

I-152 di-tert-butyl (5-(2-(4-(cyclobutylcarbamoyl)phenylamino)-5-methylpyrimidin-4-ylamino)-2-oxobenzo[d]oxazol-3(2H)-yl)methyl phosphate;

I-153 5-[2-(4-Chloro-3-trifluoromethyl-phenylamino)-5-methyl-pyrimidin-4-ylamino]-3H-benzooxazol-2-one;

I-154 5-[5-Methyl-2-(4-methyl-3-trifluoromethyl-phenylamino)-pyrimidin-4-ylamino]-3H-benzooxazol-2-one;

I-155 5-[5-Methyl-2-(4-methylsulfanyl-3-trifluoromethyl-phenylamino)-pyrimidin-4-ylamino]-3H-benzooxazol-2-one;

I-156 4-[5-Methyl-4-(2-oxo-2,3-dihydro-benzooxazol-5-ylamino)-pyrimidin-2-ylamino]-2-(4-methyl-piperidin-1-yl)-benzamide;

I-157 5-[2-(3-Cyclopentanesulfonyl-phenylamino)-5-methyl-pyrimidin-4-ylamino]-3H-benzooxazol-2-one;

I-158 5-[5-Methyl-2-(3-trifluoromethyl-phenylamino)-pyrimidin-4-ylamino]-3H-benzooxazol-2-one;

I-159 2-Methyl-4-[5-methyl-4-(2-oxo-2,3-dihydro-benzooxazol-5-ylamino)-pyrimidin-2-ylamino]-benzoic acid methyl ester;

I-160 5-[5-Methyl-2-(4-trifluoromethyl-phenylamino)-pyrimidin-4-ylamino]-3H-benzooxazol-2-one;

I-161 5-[5-Methyl-2-(4-trifluoromethoxy-3-trifluoromethyl-phenylamino)-pyrimidin-4-ylamino]-3H-benzooxazol-2-one;

I-162 5-[2-(3-Fluoro-5-trifluoromethyl-phenylamino)-5-methyl-pyrimidin-4-ylamino]-3H-benzooxazol-2-one triflouroacetate salt;

I-163 5-[2-(4-(4-Fluoro-3-trifluoromethoxy-phenylamino)-5-methyl-pyrimidin-4-ylamino]-3H-benzooxazol-2-one;

I-164 5-[5-Methyl-2-(4-methyl-3-trifluoromethyl-phenylamino)-pyrimidin-4-ylamino]-3H-benzooxazol-2-one trifluoracetic acid salt;

I-165 5-{2-[4-(2-Methoxy-ethoxy)-3-trifluoromethyl-phenylamino]-5-methyl-pyrimidin-4-ylamino}-3H-benzooxazol-2-one;

I-166 5-[2-(4-(4-Isopropyl-3-methyl-phenylamino)-5-methyl-pyrimidin-4-ylamino]-3H-benzooxazol-2-one;

I-167 5-[2-(3-Chloro-4-trifluoromethyl-phenylamino)-5-methyl-pyrimidin-4-ylamino]-3H-benzooxazol-2-one;

I-168 5-[2-(4-Ethoxy-3-trifluoromethyl-phenylamino)-5-methyl-pyrimidin-4-ylamino]-3H-benzooxazol-2-one;

I-169 5-[2-(3,5-Bis-trifluoromethyl-phenylamino)-5-methyl-pyrimidin-4-ylamino]-3H-benzooxazol-2-one;

I-170 2-Methyl-4-[5-methyl-4-(2-oxo-2,3-dihydro-benzooxazol-5-ylamino)-pyrimidin-2-ylamino]-benzoic acid;

I-171 N-Ethyl-2-methyl-4-[5-methyl-4-(2-oxo-2,3-dihydro-benzooxazol-5-ylamino)-pyrimidin-2-ylamino]-benzamide;

I-172 5-[2-(4-Chloro-phenylamino)-5-methyl-pyrimidin-4-ylamino]-3H-benzooxazol-2-one;

I-173 5-[2-(3-Chloro-phenylamino)-5-methyl-pyrimidin-4-ylamino]-3H-benzooxazol-2-one;

I-174 5-(5-Methyl-2-phenylamino-pyrimidin-4-ylamino)-3H-benzooxazol-2-one;

I-175 5-[2-(3-Bromo-phenylamino)-5-methyl-pyrimidin-4-ylamino]-3H-benzooxazol-2-one;

I-176 5-[2-(4-Chloro-2,5-dimethyl-phenylamino)-5-methyl-pyrimidin-4-ylamino]-3H-benzooxazol-2-one;

I-177 N-{4-[5-Methyl-4-(2-oxo-2,3-dihydro-benzooxazol-5-ylamino)-pyrimidin-2-ylamino]-2-trifluoromethyl-phenyl}-acetamide;

I-178 5-[2-(3,4-Dimethyl-phenylamino)-5-methyl-pyrimidin-4-ylamino]-3H-benzooxazol-2-one;

I-179 5-[2-(4-Cyclohexylmethoxy-3-trifluoromethyl-phenylamino)-5-methyl-pyrimidin-4-ylamino]- 3H-benzooxazol-2-one;

I-180 5-[2-(4-Chloro-3-trifluoromethoxy-phenylamino)-5-methyl-pyrimidin-4-ylamino]-3H-benzooxazol-2-one;

I-181 5-[2-(4-Chloro-3-methoxy-phenylamino)-5-methyl-pyrimidin-4-ylamino]-3H-benzooxazol-2-one;

I-182 5-[2-(4-Chloro-3-ethoxy-phenylamino)-5-methyl-pyrimidin-4-ylamino]-3H-benzooxazol-2-one;

I-183 5-[2-(4-Fluoro-3-methoxy-phenylamino)-5-methyl-pyrimidin-4-ylamino]-3H-benzooxazol-2-one;

I-184 5-[2-(3,5-Dichloro-phenylamino)-5-methyl-pyrimidin-4-ylamino]-3H-benzooxazol-2-one;

I-185 5-[2-(3-Bromo-5-chloro-phenylamino)-5-methyl-pyrimidin-4-ylamino]-3H-benzooxazol-2-one;

I-186 5-[2-(3-Chloro-5-fluoro-phenylamino)-5-methyl-pyrimidin-4-ylamino]-3H-benzooxazol-2-one;

I-187 3-Chloro-5-[5-methyl-4-(2-oxo-2,3-dihydro-benzooxazol-5-ylamino)-pyrimidin-2-ylamino]-benzonitrile;

I-188 5-[2-(4-Bromo-3-trifluoromethyl-phenylamino)-5-methyl-pyrimidin-4-ylamino]-3H-benzooxazol-2-one;

I-189 5-[2-(3-Bromo-5-trifluoromethyl-phenylamino)-5-methyl-pyrimidin-4-ylamino]-3H-benzooxazol-2-one;

I-190 N-Cyclobutyl-2-methyl-4-[5-methyl-4-(2-oxo-2,3-dihydro-benzooxazol-5-ylamino)-pyrimidin-2-ylamino]-benzamide;

I-191 5-{2-[3-Chloro-4-(2-morpholin-4-yl-ethoxy)-phenylamino]-5-methyl-pyrimidin-4-ylamino}-3H-benzooxazol-2-one;

I-192 5-{5-Methyl-2-[4-(2-morpholin-4-yl-ethoxy)-phenylamino]-pyrimidin-4-ylamino}-3H-benzooxazol-2-one;

I-193 5-[2-(2,4-Difluoro-5-methoxy-phenylamino)-5-methyl-pyrimidin-4-ylamino]-3H-benzooxazol-2-one;

I-194 5-[2-(3-Chloro-4-ethoxy-phenylamino)-5-methyl-pyrimidin-4-ylamino]-3H-benzooxazol-2-one;

I-195 5-[2-(4-Cyclobutylmethoxy-phenylamino)-5-methyl-pyrimidin-4-ylamino]-3H-benzooxazol-2-one;

I-196 5-[2-(4-Isobutoxy-phenylamino)-5-methyl-pyrimidin-4-ylamino]-3H-benzooxazol-2-one;

I-197 5-{5-Methyl-2-[4-(3-methyl-butoxy)-phenylamino]-pyrimidin-4-ylamino}-3H-benzooxazol-2-one;

I-198 5-[2-(3-Chloro-4-trifluoromethyl-phenylamino)-5-methyl-pyrimidin-4-ylamino]-3H-benzooxazol-2-one trifluoroacetic acid salt;

I-199 5-[2-(3-Fluoro-5-methyl-phenylamino)-5-methyl-pyrimidin-4-ylamino]-3H-benzooxazol-2-one;

I-200 5-[2-(2,4-Difluoro-3-methoxy-phenylamino)-5-methyl-pyrimidin-4-ylamino]-3H-benzooxazol-2-one; 1-201 5-(2-(4-(1-(azetidin-1-yl)ethyl)phenylamino)-5-methylpyrimidin-4-ylamino)benzo[d]oxazol-2(3H)-one;

I-202 5-(2-(4-(1-(cyclopropylamino)ethyl)phenylamino)-5-methylpyrimidin-4-ylamino)benzo [d]oxazol-2(3H)-one;

I-203 5-(5-methyl-2-(4-(1-(pyrrolidin-1-yl)ethyl)phenylamino)pyrimidin-4-ylamino)benzo [d]oxazol-2(3H)-one;

I-204 5-(5-methyl-2-(4-(1-morpholinoethyl)phenylamino)pyrimidin-4-ylamino)benzo[d]oxazol-2(3H)-one;

I-205 5-(2-(4-(1-(3-(diethylamino)pyrrolidin-1-yl)ethyl)phenylamino)-5-methylpyrimidin-4-ylamino)benzo [d]oxazol-2(3H)-one;

I-206 5-(2-(4-(1-(benzylamino)ethyl)phenylamino)-5-methylpyrimidin-4-ylamino)benzo[d]oxazol-2(3H)-one;

I-207 5-(2-(4-(1-(isopropylamino)ethyl)phenylamino)-5-methylpyrimidin-4-ylamino)benzo [d]oxazol-2(3H)-one;

I-208 5-(5-methyl-2-(3-(1-(propylamino)ethyl)phenylamino)pyrimidin-4-ylamino)benzo[d]oxazol-2(3H)-one;

I-209 5-(2-(3-(1-(isopropylamino)ethyl)phenylamino)-5-methylpyrimidin-4-ylamino)benzo[d]oxazol-2(3H)-one;

I-210 5-(2-(3-(1-(isopropylamino)ethyl)phenylamino)-5-methylpyrimidin-4-ylamino)benzo[d]oxazol-2(3H)-one;

I-211 5-(2-(3-(1-(azetidin-1-yl)ethyl)phenylamino)-5-methylpyrimidin-4-ylamino)benzo[d]oxazol-2(3H)-one;

I-212 5-(5-methyl-2-(3-(1-(pyrrolidin-1-yl)ethyl)phenylamino)pyrimidin-4-ylamino)benzo [d]oxazol-2(3H)-one;

I-213 5-(2-(3-(1-(benzylamino)ethyl)phenylamino)-5-methylpyrimidin-4-ylamino)benzo[d]oxazol-2(3H)-one;

I-214 5-(2-(3-(1-(3-(diethylamino)pyrrolidin-1-yl)ethyl)phenylamino)-5-methylpyrimidin-4-ylamino)benzo[d]oxazol-2(3H)-one;

I-215 5-(5-methyl-2-(3-(1-(piperidin-1-yl)ethyl)phenylamino)pyrimidin-4-ylamino)benzo[d]oxazol-2(3H)-one;

I-216 5-(2-(3-(1-(diethylamino)ethyl)phenylamino)-5-methylpyrimidin-4-ylamino)benzo[d]oxazol-2(3H)-one;

I-217 5-(5-methyl-2-(3-(1-morpholinoethyl)phenylamino)pyrimidin-4-ylamino)benzo[d]oxazol-2(3H)-one;

I-218 N-cyclobutyl-4-(5-methyl-4-(2-oxo-2,3-dihydrobenzo[d]oxazol-5-ylamino)pyrimidin-2-ylamino)-2-(trifluoromethyl)benzamide;

I-219 4-(5-methyl-4-(2-oxo-2,3-dihydrobenzo[d]oxazol-5-ylamino)pyrimidin-2-ylamino)-N-phenyl-2-(trifluoromethyl)benzamide;

I-220 N-cyclopropyl-2-methoxy-4-(5-methyl-4-(2-oxo-2,3-dihydrobenzo[d]oxazol-5-ylamino)pyrimidin-2-ylamino)benzamide;

I-221 2-methoxy-4-(5-methyl-4-(2-oxo-2,3-dihydrobenzo[d]oxazol-5-ylamino)pyrimidin-2-ylamino)-N-phenylbenzamide;

I-222 4-(5-methyl-4-(2-oxo-2,3-dihydrobenzo[d]oxazol-5-ylamino)pyrimidin-2-ylamino)-2-(trifluoromethyl)benzoic acid;

I-223 N-cyclopropyl-4-(5-methyl-4-(2-oxo-2,3-dihydrobenzo[d]oxazol-5-ylamino)pyrimidin-2-ylamino)-2-(trifluoromethyl)benzamide;

I-224-(2-(3-isobutoxy-5-(trifluoromethyl)phenylamino)-5-methylpyrimidin-4-ylamino)benzo[d]oxazol-2(3H)-one;

I-225 5-(2-(3-(cyclopropylmethoxy)-5-(trifluoromethyl)phenylamino)-5-methylpyrimidin-4-ylamino)benzo[d]oxazol-2(3H)-one;

I-226 5-(2-(3-cyclobutoxy-5-(trifluoromethyl)phenylamino)-5-methylpyrimidin-4-ylamino)benzo[d]oxazol-2(3H)-one;

I-227 5-(2-(3-(cyclobutylmethoxy)-5-(trifluoromethyl)phenylamino)-5-methylpyrimidin-4-ylamino)benzo[d]oxazol-2(3H)-one;

I-228 5-(2-(3-deuteratedmethoxy-5-(trifluoromethyl)phenylamino)-5-methylpyrimidin-4-ylamino)benzo[d]oxazol-2(3H)-one;

I-229 5-(2-(3-acetyl-5-methoxyphenylamino)-5-methylpyrimidin-4-ylamino)benzo[d]oxazol-2(3H)-one;

I-230 5-(2-(3-chloro-4-fluoro-5-(trifluoromethyl)phenylamino)-5-methylpyrimidin-4-ylamino)benzo[d]oxazol-2(3H)-one;

I-231 5-(2-(3-(1-(isopropylamino)ethyl)-5-methoxyphenylamino)-5-methylpyrimidin-4-ylamino)benzo[d]oxazol-2(3H)-one;

I-232 5-(2-(3-methoxy-5-(1-(propylamino)ethyl)phenylamino)-5-methylpyrimidin-4-ylamino)benzo[d]oxazol-2(3H)-one;

I-233 5-(2-(3-(1-(cyclopropylamino)ethyl)-5-methoxyphenylamino)-5-methylpyrimidin-4-ylamino)benzo[d]oxazol-2(3H)-one;

I-234 5-(2-(3-methoxy-5-(1-(pyrrolidin-1-yl)ethyl)phenylamino)-5-methylpyrimidin-4-ylamino)benzo[d]oxazol-2(3H)-one;

I-235 5-(2-(3-(1-(azetidin-1-yl)ethyl)-5-methoxyphenylamino)-5-methylpyrimidin-4-ylamino)benzo[d]oxazol-2(3H)-one;

I-236 5-(2-(3-methoxy-5-(1-(methylamino)ethyl)phenylamino)-5-methylpyrimidin-4-ylamino)benzo[d]oxazol-2(3H)-one;

I-237 5-(2-(3-(difluoromethyl)-5-methylphenylamino)-5-methylpyrimidin-4-ylamino)benzo[d]oxazol-2(3H)-one;

I-238 5-(2-(3-(fluoromethyl)-5-methylphenylamino)-5-methylpyrimidin-4-ylamino)benzo[d]oxazol-2(3H)-one;

I-239 5-(5-methyl-2-(4-methyl-3-(methylsulfonyl)phenylamino)pyrimidin-4-ylamino)benzo[d]oxazol-2(3H)-one;

I-240 5-(2-(3-fluoro-5-morpholinophenylamino)-5-methylpyrimidin-4-ylamino)benzo[d]oxazol-2(3H)-one;

I-241 5-(2-(3-fluoro-5-(4-methylpiperazin-1-yl)phenylamino)-5-methylpyrimidin-4-ylamino)benzo[d]oxazol-2(3H)-one;

I-242 5-(2-(4-fluoro-3-(methylsulfonyl)phenylamino)-5-methylpyrimidin-4-ylamino)benzo[d]oxazol-2(3H)-one;

I-244 7-methyl-5-(5-methyl-2-(3-(methylsulfonyl)phenylamino)pyrimidin-4-ylamino)benzo[d]oxazol-2(3H)-one;

I-245 5-(2-(4-fluoro-3-(methylsulfonyl)phenylamino)-5-methylpyrimidin-4-ylamino)-7-methylbenzo[d]oxazol-2(3H)-one;

I-246 5-(5-methyl-2-(3-(pyrrolidine-1-carbonyl)phenylamino)pyrimidin-4-ylamino)benzo[d]oxazol-2(3H)-one;

I-247 5-(5-methyl-2-(4-(pyrrolidine-1-carbonyl)phenylamino)pyrimidin-4-ylamino)benzoxazol-2(3H)-one;

I-249 7-fluoro-5-(5-methyl-2-(3-(methylsulfonyl)phenylamino)pyrimidin-4-ylamino)benzo[d]oxazol-2(3H)-one;

I-250 7-fluoro-5-(2-(4-fluoro-3-(methylsulfonyl)phenylamino)-5-methylpyrimidin-4-ylamino)benzo[d]oxazol-2(3H)-one;

I-251 7-fluoro-5-(2-(3-methoxy-5-(trifluoromethyl)phenylamino)-5-methylpyrimidin-4-ylamino)benzo[d]oxazol-2(3H)-one;

I-252 3-methoxy-N,N-dimethyl-5-(5-methyl-4-(2-oxo-2,3-dihydrobenzo[d]oxazol-5-ylamino)pyrimidin-2-ylamino)benzamide;

I-253 5-(2-(3-methoxy-5-(pyrrolidine-1-carbonyl)phenylamino)-5-methylpyrimidin-4-ylamino)benzo[d]oxazol-2(3H)-one;

I-254 5-(2-(3-methoxy-5-(morpholine-4-carbonyl)phenylamino)-5-methylpyrimidin-4-ylamino)benzo[d]oxazol-2(3H)-one;

I-255 5-(2-(3-methoxy-5-(4-methylpiperazine-1-carbonyl)phenylamino)-5-methylpyrimidin-4-ylamino)benzo[d]oxazol-2(3H)-one;

I-256 5-(5-methyl-2-(3-(morpholine-4-carbonyl)phenylamino)pyrimidin-4-ylamino)benzo[d]oxazol-2(3H)-one;

I-257 5-(5-methyl-2-(4-(morpholine-4-carbonyl)phenylamino)pyrimidin-4-ylamino)benzo[a]oxazol-2(3H)-one;

I-258 5-(2-(4-methoxy-3-methylphenylamino)-5-methylpyrimidin-4-ylamino)benzoxazol-2(3H)- one;

I-259 5-(2-(3-methoxy-5-methylphenylamino)-5-methylpyrimidin-4-ylamino)benzo[d]oxazol-2(3H)-one;

I-260 2-methoxy-N,N-dimethyl-5-(5-methyl-4-(2-oxo-2,3-dihydrobenzo[d]oxazol-5-ylamino)pyrimidin-2-ylamino)benzamide;

I-261 5-(2-(4-methoxy-3-(pyrrolidine-1-carbonyl)phenylamino)-5-methylpyrimidin-4-ylamino)benzo[d]oxazol-2(3H)-one;

I-262 5-(2-(4-methoxy-3-(morpholine-4-carbonyl)phenylamino)-5-methylpyrimidin-4-ylamino)benzo[d]oxazol-2(3H)-one;

I-263 5-(2-(4-methoxy-3-(4-methylpiperazine-1-carbonyl)phenylamino)-5-methylpyrimidin-4-ylamino)benzo[d]oxazol-2(3H)-one;

I-264 5-(2-(3-methyl-4-trideuteromethoxyphenylamino)-5-methylpyrimidin-4-ylamino)benzo[d]oxazol-2(3H)-one;

I-265 5-(2-(3-chloro-4-methoxy-5-methylphenylamino)-5-methylpyrimidin-4-ylamino)benzo[d]oxazol-2(3H)-one;

I-266 5-(2-(3-methyl-5-trideuteromethoxyphenylamino)-5-methylpyrimidin-4-ylamino)benzo[d]oxazol-2(3H)-one;

I-267 2-methoxy-N,N-dimethyl-4-(5-methyl-4-(2-oxo-2,3-dihydrobenzo[d]oxazol-5-ylamino)pyrimidin-2-ylamino)benzamide;

I-268 5-(2-(3-methoxy-4-(pyrrolidine-1-carbonyl)phenylamino)-5-methylpyrimidin-4-ylamino)benzo[d]oxazol-2(3H)-one;

I-269 5-(2-(3-methoxy-4-(morpholine-4-carbonyl)phenylamino)-5-methylpyrimidin-4-ylamino)benzo[d]oxazol-2(3H)-one;

I-270 5-(2-(3-methoxy-4-(4-methylpiperazine-1-carbonyl)phenylamino)-5-methylpyrimidin-4-ylamino)benzo[d]oxazol-2(3H)-one;

I-271 5-(2-(3-(difluoromethyl)-4-methoxyphenylamino)-5-methylpyrimidin-4-ylamino)benzo[d]oxazol-2(3H)-one;

I-272 5-(2-(4-methoxyphenylamino)-5-methylpyrimidin-4-ylamino)benzo[d]oxazol-2(3H)-one;

I-273 5-(2-(3-(difluoromethyl)-5-methoxyphenylamino)-5-methylpyrimidin-4-ylamino)benzo[a]oxazol-2(3H)-one;

I-274 5-(2-(3-(fluoromethyl)-5-methoxyphenylamino)-5-methylpyrimidin-4-ylamino)benzo[d]oxazol-2(3H)-one;

I-275 N2-[4-(4,4-difluoropiperidinyl)-3-fluoro]phenyl-5-methyl-N4-(2-oxo-2,3-dihydro-1,3-benzoxazol-5-yl)-2,4-pyrimidinediamine;

I-276 N2-[4-(4,4-difluoropiperidinyl)-3-trifluoromethyl]phenyl-5-methyl-N4-(2-oxo-2,3-dihydro-1,3-benzoxazol-5-yl)-2,4-pyrimidinediamine;

I-277 N2-[3-chloro-4-(4,4-difluoropiperidinyl)]phenyl-5-methyl-N4-(2-oxo-2,3-dihydro-1,3-benzoxazol-5-yl)-2,4-pyrimidinediamine;

I-278 N2-[3-chloro-4-(4-ethylpiperazino)]phenyl-5-methyl-N4-(2-oxo-2,3-dihydro-1,3-benzoxazol-5-yl)-2,4-pyrimidinediamine;

I-279 N2-[4-(4,4-difluoropiperidinyl)]phenyl-5-methyl-N4-(2-oxo-2,3-dihydro-1,3-benzoxazol-5-yl)-2,4-pyrimidinediamine;

I-280 N2-(3,5-dimethoxy)phenyl-5-methyl-N4-(2-oxo-2,3-dihydro-1,3-benzoxazol-5-yl)-2,4-pyrimidinediamine;

I-281 N2-[3-fluoro-4-(4-methylpiperazino)]phenyl-5-methyl-N4-(2-oxo-2,3-dihydro-1,3-benzoxazol-5-yl)-2,4-pyrimidinediamine;

I-282 N2-[3,5-difluoro-4-(4-methylpiperazino)]phenyl-5-methyl-N4-(2-oxo-2,3-dihydro-1,3-benzoxazol-5-yl)-2,4-pyrimidinediamine;

I-283 N2-[4-chloro-3-(4-ethylpiperazino)]phenyl-5-methyl-N4-(2-oxo-2,3-dihydro-1,3-benzoxazol-5-yl)-2,4-pyrimidinediamine;

I-284 N2-[4-chloro-3-(3,4,5-trimethylpiperazino)]phenyl-5-methyl-N4-(2-oxo-2,3-dihydro-1,3-benzoxazol-5-yl)-2,4-pyrimidinediamine;

I-285 5-methyl-N4-(2-oxo-2,3-dihydro-1,3-benzoxazol-5-yl)-N2-[3-(4-propylpiperazino)-4-trifluoromethyl]phenyl-2,4-pyrimidinediamine;

I-286 5-methyl-N2-[3-(1,3-oxazol-5-yl)]phenyl-N4-(2-oxo-2,3-dihydro-1,3-benzoxazol-5-yl)-2,4-pyrimidinediamine;

I-287 N2-(3-bromo)phenyl-5-methyl-N4-(2-oxo-2,3-dihydro-1,3-benzoxazol-5-yl)-2,4-pyrimidinediamine;

I-288 N2-(4-bromo)phenyl-5-methyl-N4-(2-oxo-2,3-dihydro-1,3-benzoxazol-5-yl)-2,4-pyrimidinediamine;

I-289 5-methyl-N4-(2-oxo-2,3-dihydro-1,3-benzoxazol-5-yl)-N2-[3-(pyridin-4-yl)]phenyl-2,4-pyrimidinediamine;

I-290 5-methyl-N4-(2-oxo-2,3-dihydro-1,3-benzoxazol-5-yl)-N2-[3-(pyridin-3-yl)]phenyl-2,4-pyrimidinediamine;

I-291 5-methyl-N4-(2-oxo-2,3-dihydro-1,3-benzoxazol-5-yl)-N2-[4-(pyridin-3-yl)]phenyl-2,4-pyrimidinediamine;

I-292 N2-[4-methoxy-3-(2-methoxyethoxy)]phenyl-5-methyl-N4-(2-oxo-2,3-dihydro-1,3-benzoxazol-5-yl)-2,4-pyrimidinediamine;

I-293 N2-[3-(cyclopropylaminocarbonylmethoxy)-4-methoxy]phenyl-5-methyl-N4-(2-oxo-2,3-dihydro-1,3-benzoxazol-5-yl)-2,4-pyrimidinediamine;

I-294 N2-(3-cyano-4-fluoro)phenyl-5-methyl-N4-(2-oxo-2,3-dihydro-1,3-benzoxazol-5-yl)-2,4-pyrimidinediamine;

I-295 N2-[3-cyano-4-(1H-pyrrol-1-yl)]phenyl-5-methyl-N4-(2-oxo-2,3-dihydro-1,3-benzoxazol-5-yl)-2,4-pyrimidinediamine;

I-296 N2-(3-methoxy-5-trifluoromethyl)phenyl-5-methyl-N4-(2-oxo-2,3-dihydro-1,3-benzoxazol-5-yl)-2,4-pyrimidinediamine;

I-297 N2-(4-methoxy-3-trifluoromethyl)phenyl-5-methyl-N4-(2-oxo-2,3-dihydro-1,3-benzoxazol-5-yl)-2,4-pyrimidinediamine;

I-298 N2-{4-methoxy-3-[(pyridin-4-yl)methoxy]}phenyl-5-methyl-N4-(2-oxo-2,3-dihydro-1,3-benzoxazol-5-yl)-2,4-pyrimidinediamine;

I-299 N2-{4-methoxy-3-[(pyridin-3-yl)methoxy]}phenyl-5-methyl-N4-(2-oxo-2,3-dihydro-1,3-benzoxazol-5-yl)-2,4-pyrimidinediamine;

I-300 N2-{4-methoxy-3-[2-(dimethylamino)ethoxy]}phenyl-5-methyl-N4-(2-oxo-2,3-dihydro-1,3-benzoxazol-5-yl)-2,4-pyrimidinediamine;

I-301 N2-[3,5-bis(trifluoromethyl)]phenyl-5-methyl-N4-(2-oxo-2,3-dihydro-1,3-benzoxazol-5-yl)-2,4-pyrimidinediamine;

I-302 N2-(3,5-dimethyl)phenyl-5-methyl-N4-(2-oxo-2,3-dihydro-1,3-benzoxazol-5-yl)-2,4-pyrimidinediamine;

I-303 N2-(4-cyano-3-trifluoromethyl)phenyl-5-methyl-N4-(2-oxo-2,3-dihydro-1,3-benzoxazol-5-yl)-2,4-pyrimidinediamine;

I-304 N2-[3-(1-hydroxy-2,2,2-trifluoroethyl)]phenyl-5-methyl-N4-(2-oxo-2,3-dihydro-1,3-benzoxazol-5-yl)-2,4-pyrimidinediamine;

I-305 N2-(3-methoxycarbonylmethoxy)phenyl-5-methyl-N4-(2-oxo-2,3-dihydro-1,3-benzoxazol-5-yl)-2,4-pyrimidinediamine;

I-306 5-methyl-N2-(3-methylaminocarbonylmethoxy)phenyl-N4-(2-oxo-2,3-dihydro-1,3-benzoxazol-5-yl)-2,4-pyrimidinediamine;

I-307 N2-(4-aminocarbonylmethoxy)phenyl-5-methyl-N4-(2-oxo-2,3-dihydro-1,3-benzoxazol-5-yl)-2,4-pyrimidinediamine;

I-308 5-methyl-N4-(2-oxo-2,3-dihydro-1,3-benzoxazol-5-yl)-N2-(4-phenylcarbonylamino)phenyl-2,4-pyrimidinediamine;

I-309 N2-[4-(N-acetyl-N-methyl)amino]phenyl-5-methyl-N4-(2-oxo-2,3-dihydro-1,3-benzoxazol-5-yl)-2,4-pyrimidinediamine;

I-310 N2-[3-cyano-4-(pyrrolidin-1-yl)]phenyl-5-methyl-N4-(2-oxo-2,3-dihydro-1,3-benzoxazol-5-yl)-2,4-pyrimidinediamine;

I-311 N2-(4-difluoromethoxy)phenyl-5-methyl-N4-(2-oxo-2,3-dihydro-1,3-benzoxazol-5-yl)-2,4-pyrimidinediamine;

I-312 N2-(3-difluoromethoxy)phenyl-5-methyl-N4-(2-oxo-2,3-dihydro-1,3-benzoxazol-5-yl)-2,4-pyrimidinediamine;

I-313 N2-(4-difluoromethoxy-3-ethoxy)phenyl-5-methyl-N4-(2-oxo-2,3-dihydro-1,3-benzoxazol-5-yl)-2,4-pyrimidinediamine;

I-314 N2-(3-chloro-4-difluoromethoxy)phenyl-5-methyl-N4-(2-oxo-2,3-dihydro-1,3-benzoxazol-5-yl)-2,4-pyrimidinediamine;

I-315 N2-[3-(cyclopropylaminocarbonylmethoxy)]phenyl-5-methyl-N4-(2-oxo-2,3-dihydro-1,3-benzoxazol-5-yl)-2,4-pyrimidinediamine;

I-316 N2-[3-aminocarbonyl-4-(4-methylpiperazino)]phenyl-5-methyl-N4-(2-oxo-2,3-dihydro-1,3-benzoxazol-5-yl)-2,4-pyrimidinediamine;

I-317 N2-[4-(isopropoxycarbonylmethoxy)]phenyl-5-methyl-N4-(2-oxo-2,3-dihydro-1,3-benzoxazol-5-yl)-2,4-pyrimidinediamine;

I-318 N2-[4-(ethylaminocarbonylamino)]phenyl-5-methyl-N4-(2-oxo-2,3-dihydro-1,3-benzoxazol-5-yl)-2,4-pyrimidinediamine;

I-319 N2-[3-(aminocarbonylmethoxy)]phenyl-5-methyl-N4-(2-oxo-2,3-dihydro-1,3-benzoxazol-5-yl)-2,4-pyrimidinediamine;

I-320 5-methyl-N2-[3-(morpholinocarbonylmethoxy)]phenyl-N4-(2-oxo-2,3-dihydro-1,3-benzoxazol-5-yl)-2,4-pyrimidinediamine;

I-321 5-methyl-N2-[3-(4-methylpiperazin-1-yl)carbonyl]phenyl-N4-(2-oxo-2,3-dihydro-1,3-benzoxazol-5-yl)-2,4-pyrimidinediamine;

I-322 5-methyl-N2-[4-(4-methylpiperazin-1-yl)carbonyl]phenyl-N4-(2-oxo-2,3-dihydro-1,3-benzoxazol-5-yl)-2,4-pyrimidinediamine;

I-323 5-methyl-N2-[3-methylaminocarbonyl-4-(4-methylpiperazino)]phenyl-N4-(2-oxo-2,3-dihydro-1,3-benzoxazol-5-yl)-2,4-pyrimidinediamine;

I-324 N2-[4-(1-aminocarbonyl-1-methypethoxy]phenyl-5-methyl-N4-(2-oxo-2,3-dihydro-1,3-benzoxazol-5-yl)-2,4-pyrimidinediamine;

I-325 5-methyl-N2-(2-methyl-3-methylaminocarbonylmethoxy)phenyl-N4-(2-oxo-2,3-dihydro-1,3-benzoxazol-5-yl)-2,4-pyrimidinediamine;

I-326 N2-(3-dimethylaminocarbonylmethoxy)phenyl-5-methyl-N4-(2-oxo-2,3-dihydro-1,3-benzoxazol-5-yl)-2,4-pyrimidinediamine;

I-327 N2-(3-cyano-4-morpholino)phenyl-5-methyl-N4-(2-oxo-2,3-dihydro-1,3-benzoxazol-5-yl)-2,4-pyrimidinediamine;

I-328 N2-(3-methoxy-2-methyl)phenyl-5-methyl-N4-(2-oxo-2,3-dihydro-1,3-benzoxazol-5-yl)-2,4-pyrimidinediamine;

I-329 N2-[3-chloro-4-(pyridin-4-yl)]phenyl 5-methyl-N4-(2-oxo-2,3-dihydro-1,3-benzoxazol-5-yl)-2,4-pyrimidinediamine;

I-330 5-methyl-N4-(2-oxo-2,3-dihydro-1,3-benzoxazol-5-yl)-N2-[4-(pyridin-4-yl)-3-trifluoromethyl]phenyl-2,4-pyrimidinediamine;

I-331 N2-[3-hydroxymethyl-4-(4-methylpiperazino)]phenyl-5-methyl-N4-(2-oxo-2,3-dihydro-1,3-benzoxazol-5-yl)-2,4-pyrimidinediamine;

I-332 5-methyl-N4-(2-oxo-2,3-dihydro-1,3-benzoxazol-5-yl)-N2-(4-piperazino)phenyl-2,4-pyrimidinediamine;

I-333 N2-[4-(4-ethylaminocarbonyl)piperazino]phenyl-5-methyl-N4-(2-oxo-2,3-dihydro-1,3-benzoxazol-5-yl)-2,4-pyrimidinediamine;

I-334 N2-[4-(1-cyano-1-methypethoxy]phenyl-5-methyl-N4-(2-oxo-2,3-dihydro-1,3-benzoxazol-5-yl)-2,4-pyrimidinediamine;

I-335 N2-[3-(1-aminocarbonyl-1-methypethoxy]phenyl-5-methyl-N4-(2-oxo-2,3-dihydro-1,3-benzoxazol-5-yl)-2,4-pyrimidinediamine;

I-336 N2-(3-methoxy-4-methoxycarbonyl)phenyl-5-methyl-N4-(2-oxo-2,3-dihydro-1,3-benzoxazol-5-yl)-2,4-pyrimidinediamine;

I-337 N2-(3-methoxy)phenyl-5-methyl-N4-(2-oxo-2,3-dihydro-1,3-benzoxazol-5-yl)-2,4-pyrimidinediamine;

I-338 5-methyl-N2-(4-morpholino)phenyl-N4-(2-oxo-2,3-dihydro-1,3-benzoxazol-5-yl)-2,4-pyrimidinediamine;

I-339 N2-(3-cyano-4-thiomorpholino)phenyl-5-methyl-N4-(2-oxo-2,3-dihydro-1,3-benzoxazol-5-yl)-2,4-pyrimidinediamine;

I-340 N2-[3-methoxy-4-(4-methylpiperazino)]phenyl-5-methyl-N4-(2-oxo-2,3-dihydro-1,3-benzoxazol-5-yl)-2,4-pyrimidinediamine;

I-341 N2-[3-cyano-4-(4-methylpiperazino)]phenyl-5-methyl-N4-(2-oxo-2,3-dihydro-1,3-benzoxazol-5-yl)-2,4-pyrimidinediamine;

I-342 N2-[3-(1-cyano-1-methypethoxy]phenyl-5-methyl-N4-(2-oxo-2,3-dihydro-1,3-benzoxazol-5-yl)-2,4-pyrimidinediamine;

I-343 N2-[4-(4-acetyppiperazino]phenyl-5-methyl-N4-(2-oxo-2,3-dihydro-1,3-benzoxazol-5-yl)-2,4-pyrimidinediamine;

I-344 N2-[4-(4-ethoxycarbonyl)piperazino]phenyl-5-methyl-N4-(2-oxo-2,3-dihydro-1,3-benzoxazol-5-yl)-2,4-pyrimidinediamine;

I-345 N2-[3-(4-acetyppiperazino]phenyl-5-methyl-N4-(2-oxo-2,3-dihydro-1,3-benzoxazol-5-yl)-2,4-pyrimidinediamine;

I-346 N2-[3-(4-ethoxycarbonyl)piperazino]phenyl-5-methyl-N4-(2-oxo-2,3-dihydro-1,3-benzoxazol-5-yl)-2,4-pyrimidinediamine;

I-347 N2-(4-difluoromethoxy-3-fluoro)phenyl-5-methyl-N4-(2-oxo-2,3-dihydro-1,3-benzoxazol-5-yl)-2,4-pyrimidinediamine;

I-348 N2-(3,5-dichloro-4-difluoromethoxy)phenyl-5-methyl-N4-(2-oxo-2,3-dihydro-1,3-benzoxazol-5-yl)-2,4-pyrimidinediamine;

I-349 N2-(4-fluoro-3-methoxy)phenyl-5-methyl-N4-(2-oxo-2,3-dihydro-1,3-benzoxazol-5-yl)-2,4-pyrimidinediamine;

I-350 N2-(3-fluoro-4-methoxy)phenyl-5-methyl-N4-(2-oxo-2,3-dihydro-1,3-benzoxazol-5-yl)-2,4-pyrimidinediamine;

I-351 N2-(3-methoxy-4-methyl)phenyl-5-methyl-N4-(2-oxo-2,3-dihydro-1,3-benzoxazol-5-yl)-2,4-pyrimidinediamine;

I-352 N2-(3-fluoro-5-methoxy)phenyl-5-methyl-N4-(2-oxo-2,3-dihydro-1,3-benzoxazol-5-yl)-2,4-pyrimidinediamine;

I-353 N2-(3-difluoromethoxy-5-trifluoromethyl)phenyl-5-methyl-N4-(2-oxo-2,3-dihydro-1,3-benzoxazol-5-yl)-2,4-pyrimidinediamine;

I-354 N2-(3-methoxy-4-trifluoromethyl)phenyl-5-methyl-N4-(2-oxo-2,3-dihydro-1,3-benzoxazol-5-yl)-2,4-pyrimidinediamine;

I-355 N2-(3,5-di-tert-butyl)phenyl-5-methyl-N4-(2-oxo-2,3-dihydro-1,3-benzoxazol-5-yl)-2,4-pyrimidinediamine;

I-356 N4-{3-[bis(1,1-dimethylethoxy)]phosphinyloxymethyl-2-oxo-2,3-dihydro-1,3-benzoxazol-5-yl}-N2-(3-methoxy-5-trifluoromethyl)phenyl-5-methyl-2,4-pyrimidinediamine;

I-357 N2-(3-methoxy-5-trifluoromethyl)phenyl-5-methyl-N4-[3-(phosphonooxy)methyl-2-oxo-2,3-dihydro-1,3-benzoxazol-5-yl]-2,4-pyrimidinediamine;

I-358 N2-(3-methoxy-5-trifluoromethyl)phenyl-5-methyl-N4-[3-(phosphonooxy)methyl-2-oxo-2,3-dihydro-1,3-benzoxazol-5-yl]-2,4-pyrimidinediamine bis-sodium salt;

I-359 N2-(3,5-difluoro)phenyl-5-methyl-N4-(2-oxo-2,3-dihydro-1,3-benzoxazol-5-yl)-2,4-pyrimidinediamine;

I-360 N2-(3-fluoro-5-trifluoromethyl)phenyl-5-methyl-N4-(2-oxo-2,3-dihydro-1,3-benzoxazol-5-yl)-2,4-pyrimidinediamine;

I-361 N2-(4-fluoro-3-trifluoromethyl)phenyl-5-methyl-N4-(2-oxo-2,3-dihydro-1,3-benzoxazol-5-yl)-2,4-pyrimidinediamine;

I-362 N2-(4-fluoro-3-methyl)phenyl-5-methyl-N4-(2-oxo-2,3-dihydro-1,3-benzoxazol-5-yl)-2,4-pyrimidinediamine;

I-363 N2-(3-fluoro-4-methyl)phenyl-5-methyl-N4-(2-oxo-2,3-dihydro-1,3-benzoxazol-5-yl)-2,4-pyrimidinediamine;

I-364 N2-(3-chloro-4-methyl)phenyl-5-methyl-N4-(2-oxo-2,3-dihydro-1,3-benzoxazol-5-yl)-2,4-pyrimidinediamine;

I-365 N2-(3,4,5-trimethyl)phenyl-5-methyl-N4-(2-oxo-2,3-dihydro-1,3-benzoxazol-5-yl)-2,4-pyrimidinediamine;

I-366 N2-(3-chloro-4-trifluoromethoxy)phenyl-5-methyl-N4-(2-oxo-2,3-dihydro-1,3-benzoxazol-5-yl)-2,4-pyrimidinediamine;

I-367 N2-(4-trifluoromethylthio)phenyl-5-methyl-N4-(2-oxo-2,3-dihydro-1,3-benzoxazol-5-yl)-2,4-pyrimidinediamine;

I-368 N2-(3-fluoro)phenyl-5-methyl-N4-(2-oxo-2,3-dihydro-1,3-benzoxazol-5-yl)-2,4-pyrimidinediamine;

I-369 N2-(3,5-dimethyl-4-methoxy)phenyl-5-methyl-N4-(2-oxo-2,3-dihydro-1,3-benzoxazol-5-yl)-2,4-pyrimidinediamine;

I-370 N2-(3-carboxamide-5-trifluoromethyl)phenyl-5-methyl-N4-(2-oxo-2,3-dihydro-1,3-benzoxazol-5-yl)-2,4-pyrimidinediamine;

I-371 N2-(3,5-diisopropyl-4-methoxy)phenyl-5-methyl-N4-(2-oxo-2,3-dihydro-1,3-benzoxazol-5-yl)-2,4-pyrimidinediamine;

I-372 N2-(3-isopropoxy-5-trifluoromethyl)phenyl-5-methyl-N4-(2-oxo-2,3-dihydro-1,3-benzoxazol-5-yl)-2,4-pyrimidinediamine;

I-373 N2-(3-cyano-4-methoxy)phenyl-5-methyl-N4-(2-oxo-2,3-dihydro-1,3-benzoxazol-5-yl)-2,4-pyrimidinediamine;

I-374 N2-(3,5-dimethyl-4-fluoro)phenyl-5-methyl-N4-(2-oxo-2,3-dihydro-1,3-benzoxazol-5-yl)-2,4-pyrimidinediamine;

I-375 N2-(4-fluoro-3-trifluoromethoxy)phenyl-5-methyl-N4-(2-oxo-2,3-dihydro-1,3-benzoxazol-5-yl)-2,4-pyrimidinediamine;

I-376 N2-(3-fluoro-4-trifluoromethoxy)phenyl-5-methyl-N4-(2-oxo-2,3-dihydro-1,3-benzoxazol-5-yl)-2,4-pyrimidinediamine;

I-377 N2-(4-chloro-3-trifluoromethoxy)phenyl-5-methyl-N4-(2-oxo-2,3-dihydro-1,3-benzoxazol-5-yl)-2,4-pyrimidinediamine;

I-378 N2-(3-chloro-5-trifluoromethoxy)phenyl-5-methyl-N4-(2-oxo-2,3-dihydro-1,3-benzoxazol-5-yl)-2,4-pyrimidinediamine;

I-379 5-methyl-N2-(3-methyl-5-trifluoromethoxy)phenyl-N4-(2-oxo-2,3-dihydro-1,3-benzoxazol-5-yl)-2,4-pyrimidinediamine;

I-380 N2-(4-cyano-3-methoxy)phenyl-5-methyl-N4-(2-oxo-2,3-dihydro-1,3-benzoxazol-5-yl)-2,4-pyrimidinediamine;

I-381 N2-(3,5-difluoro-4-methoxy)phenyl-5-methyl-N4-(2-oxo-2,3-dihydro-1,3-benzoxazol-5-yl)-2,4-pyrimidinediamine;

I-382 5-methyl-N2-(4-morpholinomethyl)phenyl-N4-(2-oxo-2,3-dihydro-1,3-benzoxazol-5-yl)-2,4-pyrimidinediamine;

I-383 N2-(4-chloro-3-cyano-5-ethyl)phenyl-5-methyl-N4-(2-oxo-2,3-dihydro-1,3-benzoxazol-5-yl)-2,4-pyrimidinediamine;

I-384 N2-[3-(2-methoxy)ethoxy-5-trifluoromethyl]phenyl-5-methyl-N4-(2-oxo-2,3-dihydro-1,3-benzoxazol-5-yl)-2,4-pyrimidinediamine;

I-385 N2-(4-difluoromethoxy-3,5-dimethyl)phenyl-5-methyl-N4-(2-oxo-2,3-dihydro-1,3-benzoxazol-5-yl)-2,4-pyrimidinediamine;

I-386 N2-[3-(1-aminocarbonyl-1-methypethoxy-4-fluoro]phenyl-5-methyl-N4-(2-oxo-2,3-dihydro-1,3-benzoxazol-5-yl)-2,4-pyrimidinediamine;

I-387 N2-(4-difluoromethoxy-3-methyl)phenyl-5-methyl-N4-(2-oxo-2,3-dihydro-1,3-benzoxazol-5-yl)-2,4-pyrimidinediamine;

I-388 N2-(3,5-difluoro-4-difluoromethoxy)phenyl-5-methyl-N4-(2-oxo-2,3-dihydro-1,3-benzoxazol-5-yl)-2,4-pyrimidinediamine;

I-389 N2-[4-(1-aminocarbonyl-1-methypethoxy-3,5-dimethyl]phenyl-5-methyl-N4-(2-oxo-2,3-dihydro-1,3-benzoxazol-5-yl)-2,4-pyrimidinediamine;

I-390 N2-(3-difluoromethoxy-4-methyl)phenyl-5-methyl-N4-(2-oxo-2,3-dihydro-1,3-benzoxazol-5-yl)-2,4-pyrimidinediamine;

I-391 N2-[4-(1-aminocarbonyl-1-methypethoxy-3-methyl]phenyl-5-methyl-N4-(2-oxo-2,3-dihydro-1,3-benzoxazol-5-yl)-2,4-pyrimidinediamine;

I-392 N2-[3-(1-aminocarbonyl-1-methypethoxy-4-methyl]phenyl-5-methyl-N4-(2-oxo-2,3-dihydro-1,3-benzoxazol-5-yl)-2,4-pyrimidinediamine;

I-393 N2-(3-methoxy-5-trifluoromethyl)phenyl-5-methyl-N4-(2-oxo-2,3-dihydro-1,3-benzoxazol-5-yl)-2,4-pyrimidinediamine besylate salt;

I-394 N2-(4-chloro-3,5-dimethyl)phenyl-5-methyl-N4-(2-oxo-2,3-dihydro-1,3-benzoxazol-5-yl)-2,4-pyrimidinediamine;

I-395 N2-[4-(1-aminocarbonyl-1-methypethoxy-3,5-difluoro]phenyl-5-methyl-N4-(2-oxo-2,3-dihydro-1,3-benzoxazol-5-yl)-2,4-pyrimidinediamine;

I-396 N2-[3-(1-methoxy-2,2,2-trifluoroethyl)]phenyl-5-methyl-N4-(2-oxo-2,3-dihydro-1,3-benzoxazol-5-yl)-2,4-pyrimidinediamine;

I-397 N2-[3-(1-cyano-1-methypethoxy-4-methyl]phenyl-5-methyl-N4-(2-oxo-2,3-dihydro-1,3-benzoxazol-5-yl)-2,4-pyrimidinediamine;

I-398 N2-(3,4-difluoro)phenyl-5-methyl-N4-(2-oxo-2,3-dihydro-1,3-benzoxazol-5-yl)-2,4-pyrimidinediamine;

I-399 N2-(3-chloro-4-fluoro)phenyl-5-methyl-N4-(2-oxo-2,3-dihydro-1,3-benzoxazol-5-yl)-2,4-pyrimidinediamine;

I-400 N2-(4-chloro-3-fluoro)phenyl-5-methyl-N4-(2-oxo-2,3-dihydro-1,3-benzoxazol-5-yl)-2,4-pyrimidinediamine;

I-401 N2-(3-difluoromethoxy-5-fluoro)phenyl-5-methyl-N4-(2-oxo-2,3-dihydro-1,3-benzoxazol-5-yl)-2,4-pyrimidinediamine;

I-402 N2-[3-(1-aminocarbonyl-1-methypethoxy-5-fluoro]phenyl-5-methyl-N4-(2-oxo-2,3-dihydro-1,3-benzoxazol-5-yl)-2,4-pyrimidinediamine;

I-403 5-(5-Methyl-2-m-tolylamino-pyrimidin-4-ylamino)-3H-benzooxazol-2-one;

I-404 5-{2-[4-(3-Dimethylamino-propoxy)-3-trifluoromethyl-phenylamino]-5-methyl-pyrimidin-4-ylamino}-3H-benzooxazol-2-one;

I-405 N4-{3-[bis(1,1-dimethylethoxy)]phosphinyloxymethyl-2-oxo-2,3-dihydro-1,3-benzoxazol-5-yl}-N2-(3,4,5-trimethyl)phenyl-5-methyl-2,4-pyrimidinediamine;

I-406 5-methyl-N4-[3-(phosphonooxy)methyl-2-oxo-2,3-dihydro-1,3-benzoxazol-5-yl]-N2-(3,4,5-trimethyl)phenyl-2,4-pyrimidinediamine;

I-407 5-methyl-N4-[3-(phosphonooxy)methyl-2-oxo-2,3-dihydro-1,3-benzoxazol-5-yl]-N2-(3,4,5-trimethyl)phenyl-2,4-pyrimidinediamine bis-sodium salt;

I-408 5-methyl-N4-(2-oxo-2,3-dihydro-1,3-benzoxazol-5-yl)-N2-(3,4,5-trifluoro)phenyl-2,4-pyrimidinediamine;

I-409 N2-(3-methoxy-5-trifluoromethyl)phenyl-5-methyl-N4-(2-oxo-2,3-dihydro-1,3-benzoxazol-5-yl)-2,4-pyrimidinediamine tosylate salt;

I-410 N2-(3-methoxy-5-trifluoromethyl)phenyl-5-methyl-N4-(2-oxo-2,3-dihydro-1,3-benzoxazol-5-yl)-2,4-pyrimidinediamine mesylate salt;

I-411 N2-(3-methoxy-5-trifluoromethyl)phenyl-5-methyl-N4-(2-oxo-2,3-dihydro-1,3-benzoxazol-5-yl)-2,4-pyrimidinediamine sulfate salt;

I-412 N2-(3-methoxy-5-trifluoromethyl)phenyl-5-methyl-N4-(2-oxo-2,3-dihydro-1,3-benzoxazol-5-yl)-2,4-pyrimidinediamine hydrogen chloride salt;

I-413 N2-(3-methoxy-5-trifluoromethyl)phenyl-5-methyl-N4-(2-oxo-2,3-dihydro-1,3-benzoxazol-5-yl)-2,4-pyrimidinediamine sodium salt;

I-414 N2-(3-methoxy-5-trifluoromethyl)phenyl-5-methyl-N4-(2-oxo-2,3-dihydro-1,3-benzoxazol-5-yl)-2,4-pyrimidinediamine choline salt;

I-415 N2-(3,5-difluoro-4-trifluoromethyl)phenyl-5-methyl-N4-(2-oxo-2,3-dihydro-1,3-benzoxazol-5-yl)-2,4-pyrimidinediamine;

I-416 N2-[3-(1-cyano-1-methypethoxy-5-fluoro]phenyl-5-methyl-N4-(2-oxo-2,3-dihydro-1,3-benzoxazol-5-yl)-2,4-pyrimidinediamine;

I-417 N2-[3-(1-cyano-1-methypethoxy-4-fluoro]phenyl-5-methyl-N4-(2-oxo-2,3-dihydro-1,3-benzoxazol-5-yl)-2,4-pyrimidinediamine;

I-418 N2-(4-chloro-3-difluoromethoxy)phenyl-5-methyl-N4-(2-oxo-2,3-dihydro-1,3-benzoxazol-5-yl)-2,4-pyrimidinediamine;

I-419 5-(2-(4-isopropylphenylamino)-5-methylpyrimidin-4-ylamino)benzo[d]oxazol-2(3H)-one;

I-420 5-(2-(4-tert-butylphenylamino)-5-methylpyrimidin-4-ylamino)benzo[d]oxazol-2(3H)-one;

I-421 5-(2-(p-toluidino)-5-methylpyrimidin-4-ylamino)benzo[d]oxazol-2(3H)-one;

I-422 5-(2-(3-(isopropoxymethyl)-4-methoxyphenylamino)-5-methylpyrimidin-4-ylamino)benzo[d]oxazol-2(3H)-one;

I-423 5-(2-(3-(1-hydroxyethyl)-5-methoxyphenylamino)-5-methylpyrimidin-4-ylamino)benzo[d]oxazol-2(3H)-one;

I-424 5-[2-(3-Chloro-4-hydroxy-phenylamino)-5-methyl-pyrimidin-4-ylamino]-3H-benzooxazol-2-one;

I-425 5-[2-(4-Hydroxy-phenylamino)-5-methyl-pyrimidin-4-ylamino]-3H-benzooxazol-2-one;

I-426 5-{2-[4-(2-Dimethylamino-ethoxy)-phenylamino]-5-methyl-pyrimidin-4-ylamino}-3H-benzooxazol-2-one;

I-427 5-(2-(3-methoxy-5-(trifluoromethyl)phenylamino)-5-methylpyrimidin-4-ylamino)-7-methylbenzo[d]oxazol-2(3H)-one;

I-428 5-(2-(3-methoxy-5-methylphenylamino)-5-methylpyrimidin-4-ylamino)-7-methylbenzo[d]oxazol-2(3H)-one;

I-429 5-(2-(4-methoxy-3-methylphenylamino)-5-methylpyrimidin-4-ylamino)-7-methylbenzo[d]oxazol-2(3H)-one;

I-430 7-fluoro-5-(2-(3-methoxy-5-methylphenylamino)-5-methylpyrimidin-4-ylamino)benzo[a]oxazol-2(3H)-one;

I-431 7-fluoro -5-(2-(4-methoxy-3-methylphenylamino)-5-methylpyrimidin-4-ylamino)benzo[a]oxazol-2(3H)-one;

I-432 5-(2-(4-fluoro-3-methoxy-5-methylphenylamino)-5-methylpyrimidin-4-ylamino)benzo[d]oxazol-2(3H)-one;

I-433 5-(2-(4-(difluoromethoxy)-3-(fluoromethyl)phenylamino)-5-methylpyrimidin-4-ylamino)benzo[d]oxazol-2(3H)-one formate salt;

I-434 N2-(4-cyano-3-difluoromethoxy)phenyl-5-methyl-N4-(2-oxo-2,3-dihydro-1,3-benzoxazol-5-yl)-2,4-pyrimidinediamine;

I-435 N2-(3-difluoromethoxy-4-fluoro)phenyl-5-methyl-N4-(2-oxo-2,3-dihydro-1,3-benzoxazol-5-yl)-2,4-pyrimidinediamine;

I-436 5-methyl-N4-(2-oxo-2,3-dihydro-1,3-benzoxazol-5-yl)-N2-(3,4,5-trimethyl)phenyl-2,4-pyrimidinediamine sodium salt;

I-437 N2-(3,5-dimethyl-4-fluoro)phenyl-5-methyl-N4-(2-oxo-2,3-dihydro-1,3-benzoxazol-5-yl)-2,4-pyrimidinediamine sodium salt;

I-438 5-(2-(3-(difluoromethyl)-4-methylphenylamino)-5-methylpyrimidin-4-ylamino)benzo[d]oxazol-2(3H)-one;

I-439 5-(2-(3-(fluoromethyl)-4-methylphenylamino)-5-methylpyrimidin-4-ylamino)benzo[d]oxazol-2(3H)-one;

I-440 5-(2-(3-(difluoromethyl)-5-methoxyphenylamino)-5-methylpyrimidin-4-ylamino)benzo[d]oxazol-2(3H)-one formate salt;

I-441 5-(2-(4-d₃-methoxy-3-(trifluoromethyl)phenylamino)-5-methylpyrimidin-4-ylamino)benzo[d]oxazol-2(3H)-one;

I-442 5-(2-(4-(difluoromethoxy)-3-(difluoromethyl)phenylamino)-5-methylpyrimidin-4-ylamino)benzo[d]oxazol-2(3H)-one formate salt;

I-443 5-(5-methyl-2-(4-methyl-3-(pyridin-4-yl)phenylamino)pyrimidin-4-ylamino)benzo[d]oxazol-2(3H)-one;

I-444 5-(5-methyl-2-(4-methyl-3-(pyridin-3-yl)phenylamino)pyrimidin-4-ylamino)benzo[d]oxazol-2(3H)- one;

I-445 5-(2-(3-acetyl-5-(trifluoromethyl)phenylamino)-5-methylpyrimidin-4-ylamino)benzo[d]oxazol-2(3H)-one;

I-446 5-(2-(3-(1-hydroxyethyl)-5-(trifluoromethyl)phenylamino)-5-methylpyrimidin-4-ylamino)benzo[d]oxazol-2(3H)-one;

I-447 5-[2-(4-d₃-Methoxy-phenylamino)-5-methyl-pyrimidin-4-ylamino]-3H-benzooxazol-2-one;

I-448 5-[2-(3-Chloro-4-d₃-methoxy-phenylamino)-5-methyl-pyrimidin-4-ylamino]-3H-benzooxazol-2-one;

I-449 5-{2-[4-(2-Diethylamino-ethoxy)-phenylamino]-5-methyl-pyrimidin-4-ylamino}-3H-benzooxazol-2-one;

I-450 N4-{3-[bis(1, 1-dimethylethoxy)]phosphinyloxymethyl-2-oxo-2,3-dihydro-1,3-benzoxazol-5-yl}-N2-(3,5-dimethyl-4-fluoro)phenyl-5-methyl-2,4-pyrimidinediamine;

I-451 N2-(3,5-dimethyl-4-fluoro)phenyl-5-methyl-N4-[3-(phosphonooxy)methyl-2-oxo-2,3-dihydro-1,3-benzoxazol-5-yl]-2,4-pyrimidinediamine bis-sodium salt;

I-452 5-(2-(3,4-dimethoxy-5-methylphenylamino)-5-methylpyrimidin-4-ylamino)benzo[d]oxazol-2(3H)-one;

I-453 5-(2-(3,4-dimethoxy-5-methylphenylamino)-5-methylpyrimidin-4-ylamino)-7-methylbenzo[d]oxazol-2(3H)-one;

I-454 5-(2-(3,4-dimethoxy-5-methylphenylamino)-5-methylpyrimidin-4-ylamino)-7-fluorobenzo[d]oxazol-2(3H)-one;

I-455 5-{2-[3-Chloro-4-(2-diethylamino-ethoxy)-phenylamino]-5-methyl-pyrimidin-4-ylamino}-3H-benzooxazol-2-one;

I-456 5-[2-(2,4-Difluoro-phenylamino)-5-methyl-pyrimidin-4-ylamino]-3H-benzooxazol-2-one;

I-457 5-(5-methyl-2-(3-(1-(methylamino)ethyl)-5-(trifluoromethyl)phenylamino)pyrimidin-4-ylamino)benzo[d]oxazol-2(3H)-one;

I-458 5-(2-(3-chloro-4,5-dimethoxyphenylamino)-5-methylpyrimidin-4-ylamino)benzo[d]oxazol-2(3H)-one;

I-459 5-(2-(3,5-dimethyl-4-(2-morpholinoethoxy)phenylamino)-5-methylpyrimidin-4-ylamino)benzo[d]oxazol-2(3H)-one;

I-460 5-(5-methyl-2-(3-(1-(methylamino)butyl)-5-(trifluoromethyl)phenylamino)pyrimidin-4-ylamino)benzo[d]oxazol-2(3H)-one;

I-461 5-(2-(3-(1-(cyclopropylamino)ethyl)-5-(trifluoromethyl)phenylamino)-5-methylpyrimidin-4-ylamino)benzo[d]oxazol-2(3H)-one;

I-462 5-(2-(3-(1-(ethylamino)ethyl)-5-(trifluoromethyl)phenylamino)-5-methylpyrimidin-4-ylamino)benzo[d]oxazol-2(3H)-one;

I-463 5-(5-methyl-2-(3-(1-(pyrrolidin-1-yl)ethyl)-5-(trifluoromethyl)phenylamino)pyrimidin-4-ylamino)benzo[d]oxazol-2(3H)-one;

I-464 5-(2-(3-(1-(azetidin-1-yl)ethyl)-5-(trifluoromethyl)phenylamino)-5-methylpyrimidin-4-ylamino)benzo[d]oxazol-2(3H)-one;

I-465 5-(2-(3-(1-(cyclobutylamino)ethyl)-5-(trifluoromethyl)phenylamino)-5-methylpyrimidin-4-ylamino)benzo[d]oxazol-2(3H)-one;

I-466 5-[2-(2,5-Difluoro-phenylamino)-5-methyl-pyrimidin-4-ylamino]-3H-benzooxazol-2-one;

I-467 5-[2-(2,3-Difluoro-phenylamino)-5-methyl-pyrimidin-4-ylamino]-3H-benzooxazol-2-one;

I-468 5-[2-(2-Fluoro-phenylamino)-5-methyl-pyrimidin-4-ylamino]-3H-benzooxazol-2-one;

I-469 N-Cyclobutyl-3-[5-methyl-4-(2-oxo-2,3-dihydro-benzooxazol-5-ylamino)-pyrimidin-2-ylamino]-5-trifluoromethyl-benzamide;

I-470 5-[2-(4-Fluoro-phenylamino)-5-methyl-pyrimidin-4-ylamino]-3H-benzooxazol-2-one;

I-471 5-(2-(4-fluoro-3-(pyridin-4-yl)phenylamino)-5-methylpyrimidin-4-ylamino)benzo[d]oxazol-2(3H)-one;

I-472 5-(2-(4-fluoro-3-(pyridin-3-yl)phenylamino)-5-methylpyrimidin-4-ylamino)benzo[d]oxazol-2(3H)-one;

I-473 5-(2-(3-(1-(isopropylamino)ethyl)-5-(trifluoromethyl)phenylamino)-5-methylpyrimidin-4-ylamino)benzo[d]oxazol-2(3H)-one;

I-474 5-(2-(3,5-dimethylphenylamino)-5-methylpyrimidin-4-ylamino)-7-methylbenzo[d]oxazol-2(3H)-one;

I-475 7-methyl-5-(5-methyl-2-(3,4,5-trimethylphenylamino)pyrimidin-4-ylamino)benzoldloxazol-2(3H)-one;

I-476 5-(2-(4-fluoro-3,5-dimethylphenylamino)-5-methylpyrimidin-4-ylamino)-7-methylbenzo[d]oxazol-2(3H)-one;

I-477 5-[5-Methyl-2-(2,3,4,5-tetrafluoro-phenylamino)-pyrimidin-4-ylamino]-3H-benzooxazol-2-one;

I-478 N2-(3-cyano-5-difluoromethoxy)phenyl-5-methyl-N4-(2-oxo-2,3-dihydro-1,3-benzoxazol-5-yl)-2,4-pyrimidinediamine;

I-479 5-methyl-N2-(3-methyl-5-trifluoromethyl)phenyl-N4-(2-oxo-2,3-dihydro-1,3-benzoxazol-5-yl)-2,4-pyrimidinediamine;

I-480 5-[5-Methyl-2-(2,3,5-trifluoro-phenylamino)-pyrimidin-4-ylamino]-3H-benzooxazol-2-one;

I-481 5-[5-Methyl-2-(2,4,5-trifluoro-phenylamino)-pyrimidin-4-ylamino]-3H-benzooxazol-2-one;

I-482 5-(5-methyl-2-(3-methyl-4-(pyridin-4-yl)phenylamino)pyrimidin-4-ylamino)benzo[a]oxazol-2(3H)-one formate salt;

I-483 5-(5-methyl-2-(3-methyl-4-(pyridin-3-yl)phenylamino)pyrimidin-4-ylamino)benzo[d]oxazol-2(3H)-one formate salt;

I-484 5-(2-(3-fluoro-4-(pyridin-4-yl)phenylamino)-5-methylpyrimidin-4-ylamino)benzoxazol-2 (3H)- one;

I-485 N2-(3,4-dimethoxy-5-trifluoromethyl)phenyl-5-methyl-N4-(2-oxo-2,3-dihydro-1,3-benzoxazol-5-yl)-2,4-pyrimidinediamine;

I-486 5-(2-(4-methoxy-3-(pyridin-4-yl)phenylamino)-5-methylpyrimidin-4-ylamino)benzo[d]oxazol-2(3H)-one formate salt;

I-487 5-(2-(4-methoxy-3-(pyridin-3-yl)phenylamino)-5-methylpyrimidin-4-ylamino)benzo[d]oxazol-2(3H)-one formate salt;

I-488 5-(2-(3,5-dimethylphenylamino)-5-methylpyrimidin-4-ylamino)-7-fluorobenzo[d]oxazol-2(3H)-one;

I-489 7-fluoro-5-(5-methyl-2-(3,4,5-trimethylphenylamino)pyrimidin-4-ylamino)benzo[d]oxazol-2(3H)-one;

I-490 7-fluoro-5-(2-(4-fluoro-3,5-dimethylphenylamino)-5-methylpyrimidin-4-ylamino)benzo[d]oxazol-2(3H)-one;

I-491 5-(2-(4-fluoro-3-methoxy-5-methylphenylamino)-5-methylpyrimidin-4-ylamino)-7-methylbenzo[d]oxazol-2(3H)-one;

I-492 7-fluoro-5-(2-(4-fluoro-3-methoxy-5-methylphenylamino)-5-methylpyrimidin-4-ylamino)benzo[d]oxazol-2(3H)-one;

I-493 N2-(3,4-dimethyl-2-fluoro)phenyl-5-methyl-N4-(2-oxo-2,3-dihydro-1,3-benzoxazol-5-yl)-2,4-pyrimidinediamine;

I-494 5-(2-(3-methoxy-4-(pyridin-4-yl)phenylamino)-5-methylpyrimidin-4-ylamino)benzo[d]oxazol-2(3H)-one formate salt;

I-495 N2-(3-chloro-5-difluoromethoxy)phenyl-5-methyl-N4-(2-oxo-2,3-dihydro-1,3-benzoxazol-5-yl)-2,4-pyrimidinediamine;

I-496 5-[2-(3-Chloro-4-methoxy-phenylamino)-5-methyl-pyrimidin-4-ylamino]-3H-benzooxazol-2-one;

I-497 5-[2-(3-Chloro-5-trifluoromethyl-phenylamino)-5-methyl-pyrimidin-4-ylamino]-3H-benzooxazol-2-one;

I-498 5-[2-(2-Methoxy-5-trifluoromethyl-phenylamino)-5-methyl-pyrimidin-4-ylamino]-3H-benzooxazol-2-one;

I-499 5-(2-(o-toluidino)-5-methylpyrimidin-4-ylamino)benzo[d]oxazol-2(3H)-one;

I-500 5-(2-(2,3-dimethylphenylamino)-5-methylpyrimidin-4-ylamino)benzo[d]oxazol-2(3H)-one;

I-501 5-(2-(2,5-dimethylphenylamino)-5-methylpyrimidin-4-ylamino)benzo[d]oxazol-2(3H)-one;

I-502 5-(2-(2-ethylphenylamino)-5-methylpyrimidin-4-ylamino)benzo[d]oxazol-2(3H)-one;

I-503 5-(2-(3-ethylphenylamino)-5-methylpyrimidin-4-ylamino)benzo[d]oxazol-2(3H)-one;

I-504 5-(2-(4-ethylphenylamino)-5-methylpyrimidin-4-ylamino)benzo[d]oxazol-2(3H)-one;

I-505 5-(2-(3-fluoro-4-(pyridin-3-yl)phenylamino)-5-methylpyrimidin-4-ylamino)benzo[d]oxazol-2(3H)-one trifluoroacetic acid salt;

I-506 5-(2-(3-methoxy-4-(pyridin-3-yl)phenylamino)-5-methylpyrimidin-4-ylamino)benzo[d]oxazol-2(3H)-one formate salt;

I-507 5-(2-(2,4-difluoro-3-methoxyphenylamino)-5-methylpyrimidin-4-ylamino)-7-ethylbenzo[d]oxazol-2(3H)-one;

I-508 5-(2-(2,4-difluoro-3-methoxyphenylamino)-5-methylpyrimidin-4-ylamino)-7-luorobenzo[d]oxazol-2(3H)-one;

I-509 5-(2-(4-(6-chloropyridin-3-yl)phenylamino)-5-methylpyrimidin-4-ylamino)benzo[a]oxazol-2(3H)-one trifluoroacetate salt;

I-510 5-(2-(4-(6-(3-(dimethylamino)propoxy)pyridin-3-yl)phenylamino)-5-methylpyrimidin-4-ylamino)benzo[d]oxazol-2(3H)-one trifluoroacetate salt;

I-511 5-(2-(4-(6-(3-(dimethylamino)propoxy)pyridin-3-yl)phenylamino)-5-methylpyrimidin-4-ylamino)benzo [d]oxazol-2(3H)-one trifluoroacetate salt;

I-512 5-(5-methyl-2-(4-(6-morpholinopyridin-3-yl)phenylamino)pyrimidin-4-ylamino)benzo[d]oxazol-2(3H)-one trifluoroacetate salt;

I-513 5-(2-(2-fluoro-3-methylphenylamino)-5-methylpyrimidin-4-ylamino)benzo[d]oxazol-2(3H)-one;

I-514 5-(2-(2-fluoro-4-methylphenylamino)-5-methylpyrimidin-4-ylamino)benzo[d]oxazol-2(3H)-one;

I-515 5-(2-(2-fluoro-5-methylphenylamino)-5-methylpyrimidin-4-ylamino)benzo[d]oxazol-2(3H)-one;

I-516 N2-(3-difluoromethoxy-5-methyl)phenyl-5-methyl-N4-(2-oxo-2,3-dihydro-1,3-benzoxazol-5-yl)-2,4-pyrimidinediamine;

I-517 5-methyl-N4-[3-(phosphonooxy)methyl-2-oxo-2,3-dihydro-1,3-benzoxazol-5-yl]-N2-(3,4,5-trimethyl)phenyl-2,4-pyrimidinediamine calcium salt;

I-518 5-[5-Methyl-2-(2-methyl-3-trifluoromethyl-phenylamino)-pyrimidin-4-ylamino]-3H-benzooxazol-2-one;

I-519 5-(2-(5-acetyl-2-fluorophenylamino)-5-methylpyrimidin-4-ylamino)benzo[d]oxazol-2(3H)-one;

I-520 5-(2-(2-chlorophenylamino)-5-methylpyrimidin-4-ylamino)benzo[d]oxazol-2(3H)-one;

I-521 5-(2-(2-chloro-5-methylphenylamino)-5-methylpyrimidin-4-ylamino)benzo[d]oxazol-2(3H)-one;

I-522 N4-(7-chloro-2-oxo-2,3-dihydro-1,3-benzoxazol-5-yl)-5-methyl-N2-(3,4,5-trimethyl)phenyl-2,4-pyrimidinediamine;

I-523 5-(2-(2-fluoro-5-(1-hydroxyethyl)phenylamino)-5-methylpyrimidin-4-ylamino)benzo[d]oxazol-2(3H)-one;

I-524 N4-{3-[bis(1,1-dimethylethoxy)]phosphinyloxymethyl-7-chloro-2-oxo-2,3-dihydro-1,3-benzoxazol-5-yl}-N2-(3,4,5-trimethyl)phenyl-5-methyl-2,4-pyrimidinediamine;

I-525 N4-[7-chloro-3-(phosphonooxy)methyl-2-oxo-2,3-dihydro-1,3-benzoxazol-5-yl]-5-methyl-N2-(3,4,5-trimethyl)phenyl-2,4-pyrimidinediamine;

I-526 5-methyl-N4-[3-(phosphonooxy)methyl-2-oxo-2,3-dihydro-1,3-benzoxazol-5-yl]-N2-(3,4,5-trimethyl)phenyl-2,4-pyrimidinediamine magnesium salt;

I-527 5-[2-(4-Iodo-3,5-dimethyl-phenylamino)-5-methyl-pyrimidin-4-ylamino]-3H-benzooxazol-2-one;

I-528 N4-[7-chloro-3-(phosphonooxy)methyl-2-oxo-2,3-dihydro-1,3-benzoxazol-5-yl]-5-methyl-N2-(3,4,5-trimethyl)phenyl-2,4-pyrimidinediamine bis-sodium salt;

I-529 5-(2-(3,5-dimethoxy-4-methylphenylamino)-5-methylpyrimidin-4-ylamino)benzo[d]oxazol-2(3H)-one;

I-530 5-(2-(2-fluoro-4,5-dimethylphenylamino)-5-methylpyrimidin-4-ylamino)benzoxazol-2(3H)-one;

I-531 5-methyl-N4-[3-(phosphonooxy)methyl-2-oxo-2,3-dihydro-1,3-benzoxazol-5-yl]-N2-(3,4,5-trimethyl)phenyl-2,4-pyrimidinediamine bis-choline salt;

I-532 5-(2-(2-fluoro-4-methyl-3-(trifluoromethyl)phenylamino)-5-methylpyrimidin-4-ylamino)benzo[d]oxazol-2(3H)-one;

I-533 5-(2-(2-fluoro-5-methoxyphenylamino)-5-methylpyrimidin-4-ylamino)benzo[d]oxazol-2(3H)-one;

I-534 5-(2-(2-fluoro-3,4,5-trimethylphenylamino)-5-methylpyrimidin-4-ylamino)benzoxazol-2(3H)- one;

I-535 5-(2-(3-methoxy-4,5-dimethylphenylamino)-5-methylpyrimidin-4-ylamino)benzo[d]oxazol-2(3H)-one;

I-536 Sodium (5-(2-(3-methoxy-5-methylphenylamino)-5-methylpyrimidin-4-ylamino)-7-methyl-2-oxobenzo[d]oxazol-3(2H)-yl)methyl phosphate;

I-537 N2-(3,4-dimethyl-5-fluoro)phenyl-5-methyl-N4-(2-oxo-2,3-dihydro-1,3-benzoxazol-5-yl)-2,4-pyrimidinediamine;

I-538 Sodium (5-(2-(4-fluoro-3-methoxy-5-methylphenylamino)-5-methylpyrimidin-4-ylamino)-2-oxobenzo[d]oxazol-3(2H)-yl)methyl phosphate;

II-1 N4-(benzoxazol-2 (3H)-on-5-yl)-N2-(6-dimethylaminopyridin-3-yl)-5-methylpyrimidine-2,4-diamine;

II-2 N4-(benzo[d]oxazol-2(3H)-on-5-yl)-N2-(6-((1S,4R)-5-methyl-2,5-diazabicyclo[2 .2.1]heptan-2-yl)-pyridin-3-yl)-5-methylpyrimidine-2,4-diamine;

II-3 N4-(benzoxazol-2 (3H)-on-5-yl)-N2-(6-(4-methyl-1 ,4-diazepan-1-yl)pyridin-3-yl)-5-methylpyrimidine-2,4-diamine;

II-4 N4-(3-n-propylbenzo[d]oxazol-2 (3H)-on-5-yl)-N2-(6-(4-methylpiperazin-1-yl)pyridin-3-yl)-5-methylpyrimidine-2,4-diamine;

II-5 N4-(benzo[d]oxazol-2(3H)-on-5-yl)-N2-(6-(4-tert-butyloxycarbonylpiperazin-1-yl)pyridin-3-yl)-5-methylpyrimidine-2,4-diamine;

II-6 N4-(benzo[d]oxazol-2 (3H)-on-5-yl)-N2-(6-(4-methylpiperidin-1-yl)pyridin-3-yl)-5-methylpyrimidine-2,4-diamine;

II-7 N2-(6-(4-methylpiperazin-1-yl)pyridin-3-yl)-N4-(3-isopropylbenzo[d]oxazol-2(3H)-on-5-yl)-5-methylpyrimidine-2,4-diamine;

II-8 N4-(benzo[d]oxazol-2(3H)-on-5-yl)-N2-(6-(4-trifluoromethoxycarbonylpiperazin-1-yl)pyridin-3-yl)-5-methylpyrimidine-2,4-diamine;

II-9 N4-(benzo[d]oxazol-2(3H)-on-5-yl)-N2-(6-(4-methoxycarbonylpiperazin-1-yl)pyridin-3-yl)-5-methylpyrimidine-2,4-diamine;

II-10 N4-(benzo[d]oxazol-2(3H)-on-5-yl)-N2-(6-(piperazin-1-yl)pyridin-3-yl)-5-methylpyrimidine-2,4-diamine;

II-11 N4-(benzo[d]oxazol-2(3H)-on-5-yl)-N2-(6-(3-methyl-4-tert-butoxycarbonylpiperazin-1-yl)pyridin-3-yl)-5-methylpyrimidine-2,4-diamine;

II-12 N4-(benzo[d]oxazol-2(3H)-on-5-yl)-N2-(6-(3-methylpiperazin-1-yl)pyridin-3-yl)-5-methylpyrimidine-2,4-diamine;

II-13 N4-(benzoxazolin-2-on-5-yl)-N2-[2-(4-methylpiperazin-1-yl)pyridin-5-yl]-5-methylpyrimidine-2,4-diamine;

II-14 N4-(benzo[d]oxazol-2(3H)-on-5-yl)-N2-[2-(4-methylpiperazin-1-yl)pyridin-5-yl]-5-fluoropyrimidine-2,4-diamine;

II-15 N4-(benzimidazolin-2-on-5-yl)-N2-[2-(4-methylpiperazin-1-yl)pyridin-5-yl]-5-methylpyrimidine-2,4-diamine;

II-16 N4-(benzimidazolin-2-on-5-yl)-N2-[2-(4-methylpiperazin-1-yl)pyridin-5-yl]-5-fluoropyrimidine-2,4-diamine;

II-17 N4-(benzo[d]oxazol-2(3H)-on-5-yl)-N2-((2-morpholinyl)pyridin-5-yl)-5-methylpyrimidine-2,4-diamine;

II-18 N4-(benzo[d]oxazol-2(3H)-on-5-yl)-N2-((2-morpholinyl)pyridin-5-yl)-5-fluoropyrimidine-2,4-diamine;

II-19 N4-(benzimidazolin-2-on-5-yl)-N2-((2-morpholinyl)pyridin-5-yl)-5-fluoropyrimidine-2,4-diamine;

II-20 N4-(1,3-dimethylbenzimidazolin-2-on-5-yl)-N2-[2-(4-methylpiperazino)pyridin-5-yl]-5-methylpyrimidine-2,4-diamine;

II-21 N4-(1,3-dimethylbenzimidazolin-2-on-5-yl)-N2-[2-(4-methylpiperazino)pyridin-5-yl]-5-fluoropyrimidine-2,4-diamine;

II-22 N2-(6-(4-methylpiperazin-1-yl)pyridin-3-yl)-N4-(3-methyl-2-oxo-2,3-dihydrobenzo[d]oxazol-5-yl)-5-methylpyrimidine-2,4-diamine;

II-23 N2-(6-(4-methylpiperazin-1-yl)pyridin-3-yl)-N4-(3-methyl-2-oxo-2,3-dihydrobenzo[d]oxazol-6-yl)-5-methylpyrimidine-2,4-diamine trifluoroacetate salt;

II-24 N2-(6-(4-methylpiperazin-1-yl)pyridin-3-yl)-N4-(3-methyl-2-oxo-2,3-dihydrobenzo[d]oxazol-5-yl)-5-fluoropyrimidine-2,4-diamine;

II-25 6-(5-methyl-2-(6-(4-methylpiperazin-1-yl)pyridin-3-ylamino)pyrimidin-4-ylamino)benzo[d]oxazol-2(3 H)-one;

II-26 N4-(benzoxazolin-2-on-5-yl)-N2-[3-methyl-2-(4-methylpiperazin-1-yl)pyridin-5-yl]-5-methylpyrimidine-2,4-diamine;

II-27 N4-(benzimidazolin-2-on-5-yl)-N2-[3-methyl-2-(4-methylpiperazin-1-yl)pyridin-5-yl]-5-methylpyrimidine-2,4-diamine;

II-28 N4-(benzoxazolin-2-on-5-yl)-N2-[3-methyl-2-((1 S,4S)-5-methyl-2,5-diazabicyclo[2.2.1]heptan-2-yl)pyridin-5-yl]-5-methylpyrimidine-2,4-diamine;

II-29 N4-(benzoxazolin-2-on-5-yl)-N2-[3-methyl-2-((1 S,4S)-5-methyl-2,5-diazabicyclo[2.2.1]heptan-2-yl)pyridin-5-yl]-5-fluoropyrimidine-2,4-diamine;

II-30 N4-(benzoxazolin-2-on-5-yl)-N2-[3-methyl-2-(4-methylpiperazin-1-yl)pyridin-5-yl]-5-fluoropyrimidine-2,4-diamine;

II-31 N4-(benzoxazolin-2-on-5-yl)-N2-[2-(1 S,4 S)-5-methyl-2,5-diazabicyclo[2.2.1]heptan-2-yl)pyridin-5-yl]-5-methylpyrimidine-2,4-diamine;

II-32 N4-(benzoxazolin-2-on-5-yl)-N2-[2-(1 S,4S)-2-oxa-5-azabicyclo[2.2.1]heptan-5-yl)pyridin-5-yl]-5-methylpyrimidine-2,4-diamine;

II-33 N4-(benzoxazolin-2-on-5-yl)-N2-[2-(1 S,4 S)-5-methyl-2,5-diazabicyclo[2.2.1]heptan-2-yl)pyridin-5-yl]-5-fluoropyrimidine-2,4-diamine;

II-34 N4-(benzoxazolin-2-on-5-yl)-N2-[2-(1 S,4S)-2-oxa-5-azabicyclo[2.2.1]heptan-5-yl)pyridin-5-yl]-5-fluoropyrimidine-2,4-diamine;

II-35 N4-(benzo[d]oxazol-2(3H)-on-5-yl)-N2-[2-(1-methylpiperidin-4-yl)aminopyridin-5-yl]-5-methylpyrimidine-2,4-diamine;

II-36 N4-(benzo[d]oxazol-2(3H)-on-5-yl)-N2-[2-(1 H-piperidin-4-yl)aminopyridin-5-yl]-5-methylpyrimidine-2,4-diamine;

II-37 N4-(benzo[d]oxazol-2(3H)-on-5-yl)-N2-[2-(8-methyl-8-aza-bicyclo[3.2.1.]oct-3-yl)aminopyridin-5-yl]-5-methylpyrimidine-2,4-diamine;

II-38 N4-(benzo[d]oxazol-2(3H)-on-5-yl)-N2-(4-(8-methyl-2,8-diazabicyclo[3.2.1]octan-2-yl)phenyl)-5-methylpyrimidine-2,4-diamine;

II-39 N4-(benzo[d]oxazolin-2(3H)-on-5-yl)-N2-[3-trifluoromethyl-2-(4-methylpiperazin-1-yl)pyridin-5-yl]-5-methylpyrimidine-2,4-diamine;

II-40 N4-(benzoxazolin-2-on-5-yl)-N2-[3-fluoro-2-((1 S,4S)-5-methyl-2,5-diazabicyclo[2.2.1]heptan-2-yl)pyridin-5-yl]-5-methylpyrimidine-2,4-diamine;

II-41 (S)-2-Methyl-4-{5-[5-methyl-4-(2-oxo-2,3-dihydro-benzooxazol-5-ylamino)-pyrimidin-2-ylamino]-pyridin-2-yl}-piperazine-1-carboxylic acid tert-butyl ester;

II-42 5-[5-Methyl-2-(pyridin-3-ylamino)-pyrimidin-4-ylamino]-3H-benzooxazol-2-one;

II-43 5-[2-(4-(6-Methanesulfonyl-pyridin-3-ylamino)-5-methyl-pyrimidin-4-ylamino]-3H-benzooxazol-2-one;

II-44 5-{5-Methyl-2-[6-((S)-3-methyl-piperazin-1-yl)-pyridin-3-ylamino]-pyrimidin-4-ylamino}-3H-benzooxazol-2-one;

II-45 5-{5-Methyl-2-[6-(piperazine-1-carbonyl)-pyridin-3-ylamino]-pyrimidin-4-ylamino}-3H-benzooxazol-2-one;

II-46 5-{2-[6-(4-Cyclopropylmethyl-piperazine-1-carbonyl)-pyridin-3-ylamino]-5-methyl-pyrimidin-4-ylamino}-3H-benzooxazol-2-one;

II-47 5-{2-[6-(4-Isobutyl-piperazine-1-carbonyl)-pyridin-3-ylamino]-5-methyl-pyrimidin-4-ylamino}-3H-benzooxazol-2-one;

II-48 5-{3-Fluoro-5-[5-methyl-4-(2-oxo-2,3-dihydro-benzooxazol-5-ylamino)-pyrimidin-2-ylamino]-pyridin-2-yl}-hexahydro-pyrrolo[3,4-c]pyrrole-2-carboxylic acid tert-butyl ester;

II-49 5-{3-Fluoro-5-[5-methyl-4-(2-oxo-2,3-dihydro-benzooxazol-5-ylamino)-pyrimidin-2-ylamino]-pyridin-2-yl}-2,5-diaza-bicyclo[2.2.1]heptane-2-carboxylic acid tert-butyl ester;

II-50 5-{2-[5-Fluoro-6-(hexahydro-pyrrolo[3,4-c]pyrrol-2-yl)-pyridin-3-ylamino]-5-methyl-pyrimidin-4-ylamino}-3H-benzooxazol-2-one;

II-51 5-{2-[6-(2,5-Diaza-bicyclo[2.2.1]hept-2-yl)-5-fluoro-pyridin-3-ylamino]-5-methyl-pyrimidin-4-ylamino}-3H-benzooxazol-2-one;

II-52 5-{2-[6-(2,5-Cyclopropylmethyl-hexahydro-pyrrolo[3,4-c]pyrrol-2-yl)-5-fluoro-pyridin-3-ylamino]-5-methyl-pyrimidin-4-ylamino}-3H-benzooxazol-2-one;

II-53 5-{2-[6-(2,5-Cyclopropanecarbonyl-hexahydro-pyrrolo[3,4-c]pyrrol-2-yl)-5-fluoro-pyridin-3-ylamino]-5-methyl-pyrimidin-4-ylamino}3H-benzooxazol-2-one;

II-54 5-{2-[6-(2,5-Cyclopropylmethyl-2,5-diaza-bicyclo[2.2.1]hept-2-yl)-5-fluoro-pyridin-3-ylamino]-5-methyl-pyrimidin-4-ylamino}-3H-benzooxazol-2-one;

II-55 (R)-5-(2-(6-(3 , 4-dimethylpiperazin-1-yl)pyridin-3-ylamino)-5-methylpyrimidin-4-ylamino)benzo[d]oxazol-2(3H)-one;

II-56 (R)-5-(2-(6-(4-(cyclopropylmethyl)-3-methylpiperazin-1-yl)pyridin-3-ylamino)-5-methylpyrimidin-4-ylamino)benzo[d]oxazol2(3H)-one;

II-57 (R)-5-(5-methyl-2-(6-(3-methyl-4-(2,2,2-trifluoroacetyppiperazin-1-yl)pyridin-3-ylamino)pyrimidin-4-ylamino)benzo[d]oxazol2(3H)-one;

II-58 (R)-diethyl 2-methyl-4-(5-(5-methyl-4-(2-oxo-2,3-dihydrobenzo[d]oxazol-5-ylamino)pyrimidin-2-ylamino)pyridin-2-yl)piperazin-1-ylphosphonate;

II-59 5-(2-(6-(4,4-difluoropiperidin-1-yl)pyridin-3-ylamino)-5-methylpyrimidin-4-ylamino)benzo[d]oxazol-2(3H)-one;

II-60 5-(2-(6-(4,4-dimethylpiperidin-1-yl)pyridin-3-ylamino)-5-methylpyrimidin-4-ylamino)benzo[d]oxazol-2(3H)-one;

II-61 5-(2-(6-(3,8-diaza-bicyclo[3.2.1]octan-3-yl)-5-methylpyridin-3-ylamino)-5-methylpyrimidin-4-ylamino)benzo[d]oxazol-2(3H)-one;

II-62 5-(5-methyl-2-(5-methyl-6-(8-acetyl)-3,8-diaza-bicyclo[3.2.1]octan-3-yl)pyridin-3-ylamino)pyrimidin-4-ylamino)benzo[d]oxazol-2(3H)-one;

II-63 5-(5-methyl-2-(5-methyl-6-(8-(2,2,2-trifluoroacetyl)-3,8-diaza-bicyclo[3.2.1]octan-3-yl)pyridin-3-ylamino)pyrimidin-4-ylamino)benzo[d]oxazol-2(3H)-one;

II-64 5-(5-methyl-2-(5-methyl-6-(8-methyl-3,8-diaza-bicyclo[3.2.1]octan-3-yl)pyridin-3-ylamino)pyrimidin-4-ylamino)benzo[d]oxazol-2(3H)-one;

II-65 tert-butyl 3-(3-methyl-5-(5-methyl-4-(2-oxo-2,3-dihydrobenzo[d]oxazol-5-ylamino)pyrimidin-2-ylamino)pyridin-2-yl)-8-azabicyclo[3.2.1]octane-8-carboxylate;

II-66 5-(2-(6-(8-aza-bicyclo[3.2.1]octan-3-yl)-5-methylpyridin-3-ylamino)-5-methylpyrimidin-4-ylamino)benzo[d]oxazol-2(3H)-one;

II-67 5-(2-(6-(8-(cyclopropylmethyl)-8-aza-bicyclo[3.2.1]octan-3-yl)-5-methylpyridin-3-ylamino)-5-methylpyrimidin-4-ylamino)benzo[d]oxazol-2(3H)-one;

II-68 methyl 3-(3-methyl-5-(5-methyl-4-(2-oxo-2,3-dihydrobenzo[d]oxazol-5-ylamino)pyrimidin-2-ylamino)pyridin-2-yl)-8-azabicyclo[3.2.1]octane-8-carboxylate;

II-69 5-(5-methyl-2-(5-methyl-6-(8-(2,2,2-trifluoroacetyl)-8-aza-bicyclo[3.2.1]octan-3-yl)pyridin-3-ylamino)pyrimidin-4-ylamino)benzo[d]oxazol-2(3H)-one;

II-70 (R)-5-(2-(6-(4-isopropyl-3-methylpiperazin-1-yl)pyridin-3-ylamino)-5-methylpyrimidin-4-ylamino)benzo[d]oxazol-2(3H)-one;

II-71 5-(5-methyl-2-(6-(pyrrolidin-1-yl)pyridin-3-ylamino)pyrimidin-4-ylamino)benzo[d]oxazol-2(3H)-one;

II-72 7-methyl-5-(5-methyl-2-(6-(4-methylpiperazin-1-yl)pyridin-3-ylamino)pyrimidin-4-ylamino)benzo[d]oxazol-2(3H)-one;

II-73 7-methyl-5-(5-methyl-2-(6-morpholinopyridin-3-ylamino)pyrimidin-4-ylamino)benzo [d]oxazol-2(3H)-one;

II-74 5-(2-(6-(cyclopropylmethylamino)pyridin-3-ylamino)-5-methylpyrimidin-4-ylamino)benzo [d]oxazol-2(3H)-one;

II-75 7-fluoro-5-(5-methyl-2-(6-(4-methylpiperazin-1-yl)pyridin-3-ylamino)pyrimidin-4-ylamino)benzo [a]oxazol-2(3H)-one;

II-76 7-fluoro-5-(5-methyl-2-(6-morpholinopyridin-3-ylamino)pyrimidin-4-ylamino)benzo[d]oxazol-2(3H)-one;

II-77 5-(2-(5-bromopyridin-3-ylamino)-5-methylpyrimidin-4-ylamino)benzo[d]oxazol-2(3H)-one;

II-79 N-(5-(5-methyl-4-(3-methyl-2-oxo-2,3-dihydrobenzo[d]oxazol-5-ylamino)pyrimidin-2-ylamino)pyridin-2-yl)methanesulfonamide;

II-80 5-(2-(6-(3-(dimethylamino)pyrrolidin-1-yl)pyridin-3-ylamino)-5-methylpyrimidin-4-ylamino)benzo[d]oxazol-2(3H)-one;

II-81 N-(1-(5-(5-methyl-4-(2-oxo-2,3-dihydrobenzo[d]oxazol-5-ylamino)pyrimidin-2-ylamino)pyridin-2-yl)pyrrolidin-3-yl)acetamide;

II-82 5-(2-(6-(3-(diethylamino)pyrrolidin-1-yl)pyridin-3-ylamino)-5-methylpyrimidin-4-ylamino)benzo[d]oxazol-2(3H)-one;

II-83 2,2,2-trifluoro-N-(1-(5-(5-methyl-4-(2-oxo-2,3-dihydrobenzo[d]oxazol-5-ylamino)pyrimidin-2-ylamino)pyridin-2-yl)pyrrolidin -3-yl)acetamide;

II-84 5-(5-methyl-2-(6-(3-morpholinopyrrolidin-1-yl)pyridin-3-ylamino)pyrimidin-4-ylamino)benzo[d]oxazol-2(3H)-one;

II-85 5-(2-(6-(4-methylpiperazin-1-yl)pyridin-3-ylamino)-5-(trifluoromethyl)pyrimidin-4-ylamino)benzo[d]oxazol-2(3H)-one;

II-86 tert-butyl 1-(5-(5-methyl-4-(2-oxo-2,3-dihydrobenzo[d]oxazol-5-ylamino)pyrimidin-2-ylamino)pyridin-2-yl)pyrrolidin-3-ylcarbamate;

II-87 (S)-tert-butyl methyl(1-(5-(5-methyl-4-(2-oxo-2,3-dihydrobenzo[d]oxazol-5-ylamino)pyrimidin-2-ylamino)pyridin-2-yl)piperidin-3-yl)carbamate;

II-88 (R)-5-(5-methyl-2-(6-(3-(methylamino)piperidin-1-yl)pyridin-3-ylamino)pyrimidin-4-ylamino)benzo[d]oxazol-2(3H)-one;

II-89 (R)-5-(2-(6-(3-(dimethylamino)pyrrolidin-1-yl)pyridin-3-ylamino)-5-methylpyrimidin-4-ylamino)benzo[d]oxazol-2(3H)-one;

II-90 (S)-5-(2-(6-(3-(dimethylamino)pyrrolidin-1-yl)pyridin-3-ylamino)-5-methylpyrimidin-4-ylamino)benzo[d]oxazol-2(3H)-one;

II-91 (R)-tert-butyl methyl(1-(5-(5-methyl-4-(2-oxo-2,3-dihydrobenzo[d]oxazol-5-ylamino)pyrimidin-2-ylamino)pyridin-2-yl)piperidin-3-yl)carbamate;

II-92 (R)-5-(5-methyl-2-(6-(3-(methylamino)piperidin-1-yl)pyridin-3-ylamino)pyrimidin-4-ylamino)benzo[d]oxazol-2(3H)-one;

II-94 5-(2-(6-(3-(cyclopropylmethylamino)pyrrolidin-1-yl)pyridin-3-ylamino)-5-methylpyrimidin-4-ylamino)benzo[d]oxazol-2(3H)-one;

II-95 (S)-5-(2-(6-((1-benzylpiperidin-3-yl)(methyl)amino)pyridin-3-ylamino)-5-methylpyrimidin-4-ylamino)benzo[d]oxazol-2(3H)-one;

II-96 1-ethyl-3-(1-(5-(5-methyl-4-(2-oxo-2,3-dihydrobenzo[d]oxazol-5-ylamino)pyrimidin-2-ylamino)pyridin-2-yl)pyrrolidin-3-yl)urea;

II-97 1-tert-butyl-3-(1-(5-(5-methyl-4-(2-oxo-2,3-dihydrobenzo[d]oxazol-5-ylamino)pyrimidin-2-ylamino)pyridin-2-yl)pyrrolidin-3-yl)urea;

II-98 1-benzyl-3-(1-(5-(5-methyl-4-(2-oxo-2,3-dihydrobenzo[d]oxazol-5-yl amino)pyrimidn -2-ylamino)pyridin-2-yl)pyrrolidin-3-yl)urea;

II-99 (S)-5-(2-(6-(1-benzylpiperidin-3-ylamino)pyridin-3-ylamino)-5-methylpyrimidin-4-ylamino)benzo[d]oxazol-2(3H)-one;

II-100 (S)-5-(2-(6-((1-benzylpiperidin-3-yl)(methyl)amino)pyridin-3-ylamino)-5-methylpyrimidin-4-ylamino)benzo[d]oxazol-2(3H)-one;

II-10 1 N-(1-(5-(5-methyl-4-(2-oxo-2,3-dihydrobenzo[d]oxazol-5-ylamino)pyrimidin-2-ylamino)pyridin-2-yl)pyrrolidin-3-yl)cyclopropanecarboxamide;

II-102 N-(1-(5-(5-methyl-4-(2-oxo-2,3-dihydrobenzo[d]oxazol-5-ylamino)pyrimidin-2-ylamino)pyridin-2-yl)pyrrolidin-3-yl)pivalamide;

II-103 (S)-5-(5-methyl-2-(6-(methyl(piperidin-3-yl)amino)pyridin-3-ylamino)pyrimidin-4-ylamino)benzo[d]oxazol-2(3H)-one;

II-104 (S)-5-(5-methyl-2-(6-(piperidin-3-ylamino)pyridin-3-ylamino)pyrimidin-4-ylamino)benzo[d]oxazol-2(3H)-one;

II-105 (S)-5-(2-(6-(1-benzylpiperidin-3-ylamino)pyridin-3-ylamino)-5-methylpyrimidin-4-ylamino)benzo[d]oxazol-2(3H)-one;

II-106 (R)-5-(2-(6-((1-benzylpiperidin-3-yl)(methyl)amino)pyridin-3-ylamino)-5-methylpyrimidin-4-ylamino)benzo[d]oxazol-2(3H)-one;

II-107 (R)-5-(5-methyl-2-(6-(piperidin-3-ylamino)pyridin-3-ylamino)pyrimidin-4-ylamino)benzo[d]oxazol-2(3H)-one;

II-108 (R)-5-(5-methyl-2-(6-(methyl(piperidin-3-yl)amino)pyridin-3-ylamino)pyrimidin-4-ylamino)benzo[d]oxazol-2(3H)-one;

109 N4-(benzo[d]oxazolin-2(3H)-one-5-yl)-5-methyl-N2-[2-((1 S,4 S)-5-methyl-2,5-diazabicyclo [2.2.1]heptan-2-yl)-3-trifluoromethylpyridine-5-yl]-2,4-pyrimidinediamine;

II-110 N4-(benzo[d]oxazolin-2(3H)-one-5-yl)-N2-[2-(4-ethylpiperazin-1-yl)-3-trifluoromethylpyridine-5-yl]-5-methyl-2,4-pyrimidinediamine;

II-111 N4-(benzo[d]oxazolin-2(3H)-one-5-yl)-N2-[3-fluoro-2-(4-methylpiperazin-1-yl)pyridine-5-yl]-5-methyl-2,4-pyrimidinediamine;

II-112 N4-(benzo[d]oxazolin-2(3H)-one-5-yl)-N2-{2-[(8S)-1,4-diazabicyclo[4.3.0]nonane-1-yl]-3-fluoropyridine-5-yl}-5-methyl-2,4-pyrimidinediamine;

II-113 N4-(benzo[d]oxazolin-2(3H)-one-5-yl)-N2-{2-[(8R)-1,4-diazabicyclo[4.3.0]nonane-1-yl]-3-fluoropyridine-5-yl}-5-methyl-2,4-pyrimidinediamine;

114 N4-(benzo[d]oxazolin-2(3H)-one-5-yl)-N2-[2-(4-ethylpiperazin-1-yl)-3-fluoropyridine-5-yl]-5-methyl-2,4-pyrimidinediamine;

II-115 N4-(benzo[d]oxazolin-2(3H)-one-5-yl)-N2-[3-cyano-2-41S,4S)-5-methyl-2,5-diazabicyclo[2.2.1]heptan-2-yl)pyridine-5-yl]-5-methyl-2,4-pyrimidinediamine;

II-116 N2-[3-chloro-2-(4-methylpiperazino)pyridin-5-yl]-5-methyl-N4-(2-oxo-2,3-dihydro-1,3-benzoxazol-5-yl)-2,4-pyrimidinediamine;

II-117 5-methyl-N4-(2-oxo-2,3-dihydro-1,3-benzoxazol-5-yl)-N2-[2-(1,3,5-trimethyl-3,7-diazabicyclo [3.3.1]nonan-7-yl)pyridin-5-yl]-2,4-pyrimidinediamine;

II-118 N2-[3-chloro-2-(3-ethyl-3,7-diazabicyclo[3.3.0]octan-7-yl)pyridin-5-yl]-5-methyl-N4-(2-oxo-2,3-dihydro-1,3-benzoxazol-5- yl)-2,4-pyrimidinediamine;

II-119 N2-[2-(3-ethyl-3,7-diazabicyclo[3.3.0]octan-7-yl)-3-trifluoromethylpyridin-5-yl]-5-methyl-N4-(2-oxo-2,3-dihydro-1,3-benzoxazol-5-yl)-2,4-pyrimidinediamine;

II-120 5-methyl-N2-[2-(3-methyl-3,7-diazabicyclo[3 .3.0]octan-7-yl)pyridin-5-yl]-N4-(2-oxo-2,3-dihydro-1,3-benzoxazol-5-yl)-2,4-pyrimidinediamine;

II-121 5-methyl-N2-[2-(octahydroisoindol-1-yl)pyridin-5-yl]-N4-(2-oxo-2,3-dihydro-1,3-benzoxazol-5-yl)-2,4-pyrimidinediamine;

II-122 N2-[3-chloro-2-(octahydroisoindol-1-yl)pyridin-5-yl]-5-methyl-N4-(2-oxo-2,3-dihydro-1,3-benzoxazol-5-yl)-2,4-pyrimidinediamine;

II-123 N2-(2-methoxypyridin-5-yl)-5-methyl-N4-(2-oxo-2,3-dihydro-1,3-benzoxazol-5-yl)-2,4-pyrimidinediamine;

II-124 N2-[2-(S-1,4-diazabicylco[4.3.0]nonan-4-yl)-3-trifluoromethylpyridin-5-yl]-5-methyl-N4-(2-oxo-2,3-dihydro-1,3-benzoxazol-5-yl)-2,4-pyrimidinediamine;

II-125 N2-[2-(1,4-diazabicyclo[3.2.2]nonan-4-yl)-3-fluoropyridin-5-yl]-5-methyl-N4-(2-oxo-2,3-dihydro-1,3-benzoxazol-5-yl)-2,4-pyrimidinediamine;

II-126 N2-[2-(4R-hydroxy-2-methylidene-pyrrolidin-1-yl)pyridin-5-yl]-5-methyl-N4-(2-oxo-2,3-dihydro-1,3-benzoxazol-5-yl)-2,4-pyrimidinediamine;

II-127 5-methyl-N4-(2-oxo-2,3-dihydro-1,3-benzoxazol-5-yl)-N2-[2-(cis-3,4,5-trimethylpiperazino)pyridin-5-yl]-2,4-pyrimidinediamine;

II-128 N2-[2-(1,4-diazabicylco[4.4.0]decan-4-yl)pyridin-5-yl]-5-methyl-N4-(2-oxo-2,3-dihydro-1,3-benzoxazol-5-yl)-2,4-pyrimidinediamine;

II-129 5-methyl-N4-(2-oxo-2,3-dihydro-1,3-benzoxazol-5-yl)-N2-[2-(trans-2,4,5-trimethylpiperazino)pyridin-5-yl]-2,4-pyrimidinediamine;

II-130 N2-[2-(trans-2,5-dimethylpiperazino)pyridin-5-yl]-5-methyl-N4-(2-oxo-2,3-dihydro-1,3-benzoxazol-5-yl)-2,4-pyrimidinediamine;

II-131 N2-[2-(cis-3,5-dimethylpiperazino)pyridin-5-yl]-5-methyl-N4-(2-oxo-2,3-dihydro-1,3-benzoxazol-5-yl)-2,4-pyrimidinediamine;

II-132 N2-[2-(R-1,4-diazabicyclo[4.3.0]nonan-4-yl)pyridin-5-yl]-5-methyl-N4-(2-oxo-2,3-dihydro-1,3-benzoxazol-5-yl)-2,4-pyrimidinediamine;

II-133 5-methyl-N2-[2-(7-methyl-2,7-diazaspiro[4 .4]nonan-2-yl)pyridin-5-yl]-N4-(2-oxo-2,3-dihydro-1,3-benzoxazol-5-yl)-2,4-pyrimidinediamine;

II-134 5-methyl-N2-[2-(3 S-methylmorpholino)pyridin-5-yl]-N4-(2-oxo-2,3-dihydro-1,3-benzoxazol-5-yl)-2,4-pyrimidinediamine;

II-135 5-methyl-N2-[2-(2R-methylmorpholino)pyridin-5-yl]-N4-(2-oxo-2,3-dihydro-1,3-benzoxazol-5-yl)-2,4-pyrimidinediamine;

II-136 N2-[2-(4-isopropylpiperazino)pyridin-5-yl]-5-methyl-N4-(2-oxo-2,3-dihydro-1,3-benzoxazol-5-yl)-2,4-pyrimidinediamine;

II-137 N2-[2-(3—N,N-dimethylamino-8-azabicyclo[3.2.1]octan-8-yl)pyridin-5-yl]-5-methyl-N4-(2-oxo-2,3-dihydro-1,3-benzoxazol-5-yl)-2,4-pyrimidinediamine;

II-138 5-methyl-N2-[2-(2 S-methylmorpholino)pyridin-5-yl]-N4-(2-oxo-2,3-dihydro-1,3-benzoxazol-5-yl)-2,4-pyrimidinediamine;

II-139 5-methyl-N2-{2-[(1R,4R)-2-oxa-5-azabicyclo[2 .2 . 1]heptan-5-yl]pyridin-5-yl}-N4-(2-oxo-2,3-dihydro-1,3-benzoxazol-5-yl)-2,4-pyrimidinediamine;

II-140 N2-(2,3-dimethoxypyridin-5-yl)-5-methyl-N4-(2-oxo-2,3-dihydro-1,3-benzoxazol-5-yl)-2,4-pyrimidinediamine;

II-141 N2-(2-methoxy-3-methylpyridin-5-yl)-5-methyl-N4-(2-oxo-2,3-dihydro-1,3-benzoxazol-5-yl)-2,4-pyrimidinediamine;

II-142 N2-[2-(2-hydroxy)ethoxypyridin-5-yl]-5-methyl-N4-(2-oxo-2,3-dihydro-1,3-benzoxazol-5-yl)-2,4-pyrimidinediamine;

II-143 N2-[4-methyl-2-(4-methylpiperazino)pyridin-5-yl]-5-methyl-N4-(2-oxo-2,3-dihydro-1,3-benzoxazol-5-yl)-2,4-pyrimidinediamine;

II-144 N2-(2-isopropoxypyridin-5-yl)-5-methyl-N4-(2-oxo-2,3-dihydro-1,3-benzoxazol-5-yl)-2,4-pyrimidinediamine;

II-145 N2-[2-(2-methoxy)ethoxypyridin-5-yl]-5-methyl-N4-(2-oxo-2,3-dihydro-1,3-benzoxazol-5-yl)-2,4-pyrimidinediamine;

II-146 N2-[2-(1-aminocarbonyl-1-methypethoxypyridin-5-yl]-5-methyl-N4-(2-oxo-2,3-dihydro-1,3-benzoxazol-5-yl)-2,4-pyrimidinediamine;

II-147 N2-(2-methoxy-3-trifluoromethylpyridin-5-yl)-5-methyl-N4-(2-oxo-2,3-dihydro-1,3-benzoxazol-5-yl)-2,4-pyrimidinediamine;

II-148 N2-[2-(3-hydroxy)propoxypyridin-5-yl]-5-methyl-N4-(2-oxo-2,3-dihydro-1,3-benzoxazol-5-yl)-2,4-pyrimidinediamine;

II-149 N2-[2-(3-methoxy)propoxypyridin-5-yl]-5-methyl-N4-(2-oxo-2,3-dihydro-1,3-benzoxazol-5-yl)-2,4-pyrimidinediamine;

II-150 5-(2-(6-(1,4-diazabicyclo[3.2.2]nonan-4-yl)-5-chloropyridin-3-ylamino)-5-methylpyrimidin-4-ylamino)benzo[d]oxazol-2(3H)-one;

II-151 5-[5-Methyl-4-(2-oxo-2,3-dihydro-benzooxazol-5-ylamino)-pyrimidin-2-ylamino]-pyridine-2-carboxylic acid cyclobutylamide;

II-152 N2-(5-methoxypyridin-3-yl)-5-methyl-N4-(2-oxo-2,3-dihydro-1,3-benzoxazol-5-yl)-2,4-pyrimidinediamine; II-15 3 N2-(2,3-dimethylpyridin-5-yl)-5-methyl-N4-(2-oxo-2,3-dihydro-1,3-benzoxazol-5-yl)-2,4-pyrimidinediamine;

III-1 N4-(benzo[d]oxazol-2(3H)-on-5-yl)-N2-(isoindolin-5-yl)-5-methylpyrimidine-2,4-diamine;

III-2 N4-(benzo[d]oxazol-2(3H)-on-5-yl)-N2-(2Hydroxyisoindolin-5-yl)-5-methylpyrimidine-2,4-diamine;

III-3 N4-(benzo[d]oxazol-2(3H)-on-5-yl)-N2-(2-tert-butoxoxycarbonylisoindolin-5-yl)-5-methylpyrimidine-2,4-diamine;

III-4 N4-(benzo[d]oxazol-2(3H)-on-5-yl)-N2-(2-methylisoindolin-5-yl)-5-methylpyrimidine-2,4-diamine;

III-5 N4-(benzo[d]oxazol-2(3H)-on-5-yl)-N2-(2-ethylisoindolin-5-yl)-5-methylpyrimidine-2,4-diamine;

III-6 N4-(benzo[d]oxazol-2(3H)-on-5-yl)-N2-(2-n-propylisoindolin-5-yl)-5-methylpyrimidine-2,4-diamine;

III-7 N4-(benzo[d]oxazol-2(3H)-on-5-yl)-N2-(2-cyclopropylmethylylisoindolin-5-yl)-5-methylpyrimidine-2,4-diamine;

III-8 N4-(benzo[d]oxazol-2(3H)-on-5-yl)-N2-(2-isobutylisoindolin-5-yl)-5-methylpyrimidine-2,4-diamine;

III-9 N4-(benzo[d]oxazol-2(3H)-on-5-yl)-N2-(2-isopentylisoindolin-5-yl)-5-methylpyrimidine-2,4-diamine;

III-10 N4-(benzo[d]oxazol-2(3H)-on-5-yl)-N2-(2-cyclopentylmethylisoindolin-5-yl)-5-methylpyrimidine-2,4-diamine;

III-11N4-(benzo[d]oxazol-2(3H)-on-5-yl)-N2-(2-(bicyclo[2.2.1]heptan-2-ylmethyl)isoindolin-5-371)-5-methylpyrimidine-2,4-diamine;

III-12 5-[2-(2-Acetyl-2,3-dihydro-1H-isoindol-5-ylamino)-5-methyl-pyrimidin-4-ylamino]-3H-benzooxazol-2-one;

III-13 N-{2[2(2,2-Dimethyl-propionyl)-2,3-dihydro-1H-isoindol-5-ylamino]-5-methyl-pyrimidin-4-yl}-N-[3-(2,2-dimethyl-propionyl)-2-oxo-2,3-dihydro-benzooxazol-5-yl]-2,2-dimethyl-propionamide;

III-14 5-[2-(2-Methane sulfonyl-2,3-dihydro-1H-isoindol-5-ylamino)-5-methyl-pyrimidin-4-ylamino]-3H-benzooxazol-2-one;

IV-1 N4-(benzo[d]oxazol-2(3H)-on-5-yl)-N2-(7-(pyrrolidin-1-yl)-6,7, 8,9-tetrahydro-5H-benzo[7]annulen-2-yl)-5-methylpyrimidine-2,4-diamine;

IV-2 N4-(benzo[d]oxazol-2(3H)-on-5-yl)-N2-(6,7, 8,9-tetrahydro-5H-benzo[7]annulen-5-on-3-yl)-5-methylpyrimidine-2,4-diamine;

IV-3 N4-(benzo[d]oxazol-2(3H)-on-5-yl)-N2-(6-(4-methylpiperazin-1-yl)pyridazin-3-yl)-5-methylpyrimidine-2,4-diamine;

IV-4 N4-(benzo[d]oxazol-2(3H)-on-5-yl)-N2-(1H-indazol-6-yl)-5-methylpyrimidine-2,4-diamine;

IV-5 N4-(benzo[d]oxazol-2(3H)-on-5-yl)-N2-(1,2-benzisoxazol-6-yl)-5-methylpyrimidine-2,4-diamine;

IV-6 N4-(benzo[d]oxazol-2(3H)-on-5-yl)-N2-(1H-indazol-5-yl)-5-methylpyrimidine-2,4-diamine;

IV-7 N4-(benzo[d]oxazol-2(3H)-on-5-yl)-N2-[2-(piperazino)pyridin-4-yl]-5-methylpyrimidine-2,4-diamine;

IV-8 N4-(benzo[d]oxazol-2(3H)-on-5-yl)-N2-[2-(4-methylpiperazino)pyridin-4-yl]-5-methylpyrimidine-2,4-diamine;

IV-9 N4-(benzo[d]oxazol-2(3H)-on-5-yl)-N2-(3-methyl-1,2-benzisoxazol-5-yl)-5-methylpyrimidine-2,4-diamine;

IV-10 (Z)-2-Methyl-9-[5-methyl-4-(2-oxo-2,3-dihydro-benzooxazol-5-ylamino)-pyrimidin-2-ylamino]-3,6-dihydro-2H-benzo[c] azocin-1-one;

IV-115-[2-(2,2-Difluoro-benzo[1,3]dioxol-4-ylamino)-5-methyl-pyrimidin-4-ylamino]-3H-benzooxazol-2-one;

IV-12 5-[2-(9-Isopropylamino-6,7,8,9-tetrahydro-5H-benzocyclohepten-2-ylamino)-5-methyl-pyrimidin-4-ylamino]-3H-benzooxazol-2-one;

IV-13 5-{2-[9-(3-Diethylamino-pyrrolidin-1-yl)-6,7,8,9-tetrahydro-5H-benzocyclohepten-2-ylamino]-5-methyl-pyrimidin-4-ylamino}-3H-benzooxazol-2-one;

IV-14 2-Methyl-9-[5-methyl-4-(2-oxo-2,3-dihydro-benzooxazol-5-ylamino)-pyrimidin-2-ylamino]-3,4,5,6-tetrahydro-2H-benzo[c] azocin-1-one;

IV-15 6-[5-Methyl-4-(2-oxo-2,3-dihydro-benzooxazol-5-ylamino)-pyrimidin-2-ylamino]-3,4-dihydro-2H-isoquinolin-1-one;

IV-16 5-[2-(2,2-Dioxo-1H-benzo[e] [1,3,4]oxathiazin-7-ylamino)-5-methyl-pyrimidin-4-ylamino]-3H-benzooxazol-2-one;

IV-17 5-[2-(2,2-Dimethyl-benzo[1,3]dioxol-5-ylamino)-5-methyl-pyrimidin-4-ylamino]-3H-benzooxazol-2-one;

IV-18 (Z)-5-(5-methyl-2-(1-oxo-2,3-dihydro-1H-benzo[c]azepin-7-ylamino)pyrimidin-4-ylamino)benzo[d]oxazol-2(3H)-one;

IV-19 (Z)-5-(5-methyl-2-(2-methyl-1-oxo-2,3-dihydro-1H-benzo[c]azepin-7-ylamino)pyrimidin-4-ylamino)benzo[d]oxazol-2(3H)-one;

IV-20 (Z)-5-(5-methyl-2-(2-methyl-1-oxo-2,3-dihydro-1H-benzo[c]azepin-7-ylamino)pyrimidin-4-ylamino)benzo[d]oxazol-2(3H)-one;

IV-21 5-(5-methyl-2-(2-methyl-1-oxo-2,3,4,5-tetrahydro-1H-benzo[c]azepin-7-ylamino)pyrimidin-4-ylamino)benzo[d]oxazol-2(3H)-one;

IV-22 5,5′-(5-methylpyrimidine-2,4-diyl)bis(azanediyl)dibenzo[d]oxazol-2(3H)-one

IV-23 5-(5-methyl-2-(2-oxo-2,3,4,5-tetrahydro-1H-benzo[b]azepin-8-ylamino)pyrimidin-4-ylamino)benzo [d]oxazol-2(3H)-one;

IV-24 5-(5-methyl-2-(2-oxo-1,2,3,4-tetrahydroquinolin-7-ylamino)pyrimidin-4-ylamino)benzo[d]oxazol-2(3H)-one;

IV-25 6-(5-methyl-4-(2-oxo-2,3-dihydrobenzo[d]oxazol-5-ylamino)pyrimidin-2-ylamino)-2H-benzo[b] [1,4]oxazin-3 (4H)-one;

IV-26 5-(2-(3,3-dimethyl-2-oxoindolin-6-ylamino)-5-methylpyrimidin-4-ylamino)benzoxazol-2(3H)- one;

IV-27 5-(5-methyl-2-(1-methyl-2-oxoindolin-5-ylamino)pyrimidin-4-ylamino)benzo[d]oxazol-2(3H)-one;

IV-28 5-(5-methyl-2-(1-methyl-2-oxo-2,3-dihydro-1H-benzo[a]imidazol-5-ylamino)pyrimidin-4-ylamino)benzo[d]oxazol-2(3H)-one;

IV-29 5-(5-methyl-2-(2-oxo-2,3-dihydro-1H-benzo[d]imidazol-5-ylamino)pyrimidn-4-ylamino)benzo[d]oxazol-2(3H)-one;

IV-30 5-(5-methyl-2-(1-methyl-2-oxo-2,3,4,5-tetrahydro-1H-benzo[b]azepin-8-ylamino)pyrimidin-4-ylamino)benzo[d]oxazol-2(3H)-one;

IV-31 5-(5-methyl-2-(2-oxo-2,3,4,5-tetrahydro-1H-benzo[b]azepin-7-ylamino)pyrimidin-4-ylamino)benzo[d]oxazol-2(3H)-one;

IV-32 7-(5-methyl-4-(2-oxo-2,3-dihydrobenzo[d]oxazol-5-ylamino)pyrimidin-2-ylamino)-2H-benzo[b] [1,4]oxazin-3 (4H)-one;

IV-33 5-(5-methyl-2-(2-oxo-1,2,3,4-tetrahydroquinolin-6-ylamino)pyrimidin-4-ylamino)benzo[d]oxazol-2(3H)-one;

IV-34 5-(5-methyl-2-(1-methyl-2-oxo-2,3,4,5-tetrahydro-1H-benzo[b]azepin-7-ylamino)pyrimidin-4-ylamino)benzo[d]oxazol-2(3H)-one;

IV-35 5-methyl-N2-(3,4-methylenedioxy)phenyl-N4-(2-oxo-2,3-dihydro-1,3-benzoxazol-5-yl)-2,4-pyrimidinediamine;

IV-36 N2-(2,2-difluoro-2H-1,3-benzodioxol-5-yl)-5-methyl-N4-(2-oxo-2,3-dihydro-1,3-benzoxazol-5-yl)-2,4-pyrimidinediamine;

IV-37 N2-(3,4-ethylenedioxy)phenyl-5-methyl-N4-(2-oxo-2,3-dihydro-1,3-benzoxazol-5-yl)-2,4-pyrimidinediamine;

IV-38 N2-(2,2-dimethyl-2H-1,3-benzodioxol-5-yl)-5-methyl-N4-(2-oxo-2,3-dihydro-1,3-benzoxazol-5-yl)-2,4-pyrimidinediamine;

IV-39 N2-[spiro(2,1′-cyclohexan)-1,3-benzodioxol-5-yl]-5-methyl-N4-(2-oxo-2,3-dihydro-1,3-benzoxazol-5-yl)-2,4-pyrimidinediamine;

IV-40 N2-(1,3-dimethyl-1H-pyrazolo[3,4-b]pyridin-5-yl)-5-methyl-N4-(2-oxo-2,3-dihydro-1,3-benzoxazol-5-yl)-2,4-pyrimidinediamine;

IV-41 5-methyl-N2-(1-methylindazol-6-yl)-N4-(2-oxo-2,3-dihydro-1,3-benzoxazol-5-yl)-2,4-pyrimidinediamine;

IV-42 5-methyl-N2-(1-methylindazol-5-yl)-N4-(2-oxo-2,3-dihydro-1,3-benzoxazol-5-yl)-2,4-pyrimidinediamine;

IV-43 5-methyl-N2-(3-methylisoxazolo[5,4-b]pyridin-5-yl)-N4-(2-oxo-2,3-dihydro-1,3-benzoxazol-5-yl)-2,4-pyrimidinediamine;

IV-44 N2-[4-(2-methoxyethyl)-2H-1,4-benzoxazin-3(4H)-one-7-yl]-5-methyl-N4-(2-oxo-2,3-dihydro-1,3-benzoxazol-5-yl)-2,4-pyrimidinediamine;

IV-45 N2-[2,2-dimethyl-4-(2-methoxyethyl)-2H-pyrido [3,2-b] [1,4]oxazin-3 (4H)-one-7-yl]-5-methyl-N4-(2-oxo-2,3-dihydro-1,3-benzoxazol-5-yl)-2,4-pyrimidinediamine;

IV-46 5-methyl-N4-(2-oxo-2,3-dihydro-1,3-benzoxazol-5-yl)-N2-(2H-pyrido [3,2-b] [1,4]oxazin-3 (4H)-one-7-yl)-2,4-pyrimidinediamine;

IV-47 5-methyl-N4-(2-oxo-2,3-dihydro-1,3-benzoxazol-5-yl)-N2-(2H-pyrido [3,2-b] [1,4]oxazin-3 (4H)-one-6-yl)-2,4-pyrimidinediamine;

IV-48 5-methyl-N2-(3-methylindazol-6-yl)-N4-(2-oxo-2,3-dihydro-1,3-benzoxazol-5-yl)-2,4-pyrimidinediamine;

IV-49 5-methyl-N2-(3-methylindazol-5-yl)-N4-(2-oxo-2,3-dihydro-1,3-benzoxazol-5-yl)-2,4-pyrimidinediamine;

IV-50 N2-[2,2-dimethyl-2H-1,4-benzoxazin-3(4H)-one-7-yl]-5-methyl-N4-(2-oxo-2,3-dihydro-1,3-benzoxazol-5-yl)-2,4-pyrimidinediamine;

IV-51 5-(5-methyl-2-(6-methylpyridin-2-ylamino)pyrimidin-4-ylamino)benzo[d]oxazol-2(3H)-one;

IV-52 5-(5-methyl-2-(5-methylpyridin-2-ylamino)pyrimidin-4-ylamino)benzo[d]oxazol-2(3H)-one;

IV-53 5-[2-(Isoquinolin-6-ylamino)-5-methyl-pyrimidin-4-ylamino]-3H-benzooxazol-2-one;

IV-54 5-[5-Methyl-2-(naphthalen-2-ylamino)-pyrimidin-4-ylamino]-3H-benzooxazol-2-one;

IV-55 5-[2-(4-Methoxy-naphthalen-2-ylamino)-5-methyl-pyrimidin-4-ylamino]-3H-benzooxazol-2-one;

IV-56 5-[2-(4-Hydroxy-naphthalen-2-ylamino)-5-methyl-pyrimidin-4-ylamino]-3H-benzooxazol-2-one;

IV-57 5-[2-(Isoquinolin-7-ylamino)-5-methyl-pyrimidin-4-ylamino]-3H-benzooxazol-2-one;

IV-58 N2-(4-methoxypyridin-2-yl)-5-methyl-N4-(2-oxo-2,3-dihydro-1,3-benzoxazol-5-yl)-2,4-pyrimidinediamine;

IV-59 5-[5-Methyl-2-(2,4,6-trifluoro-phenylamino)-pyrimidin-4-ylamino]-3H-benzooxazol-2-one;

IV-60 5-(2-(2,6-dimethylphenylamino)-5-methylpyrimidin-4-ylamino)benzo[d]oxazol-2(3H)-one;

IV-61 5-[5-Methyl-2-(2,4,6-trimethyl-phenylamino)-pyrimidin-4-ylamino]-3H-benzooxazol-2-one;

IV-62 5-(2-(2-fluoro-6-methylphenylamino)-5-methylpyrimidin-4-ylamino)benzo[d]oxazol-2(3H)-one;

IV-63 N2-(3-fluoropyridin-4-yl)-5-methyl-N4-(2-oxo-2,3-dihydro-1,3-benzoxazol-5-yl)-2,4-pyrimidinediamine; or

IV-64 N2-(3-fluoropyridin-4-yl)-5-methyl-N4-(2-oxo-2,3-dihydro-1,3-benzoxazol-5-yl)-2,4-pyrimidinediamine trifluoroacetic acid salt.

In particular embodiments, the compound is

N2-(3,4,5-trimethyl)phenyl-5-methyl-N4-(2-oxo-2,3-dihydro-1,3-benzoxazol-5-yl)-2,4-pyrimidinediamine, or a pharmaceutically acceptable salt thereof,

5-methyl-N4-[3-(phosphonooxy)methyl-2-oxo-2,3-dihydro-1,3-benzoxazol-5-yl]-N2-(3,4,5-trimethyl)phenyl-2,4-pyrimidinediamine, or pharmaceutically acceptable salt thereof, preferably 5-methyl-N4-[3-(phosphonooxy)methyl-2-oxo-2,3-dihydro-1,3-benzoxazol-5-yl]-N2-(3,4,5-trimethyl)phenyl-2,4-pyrimidinediamine bis-sodium salt;

N2-(3-methoxy-5-trifluoromethyl)phenyl-5-methyl-N4-[3-(phosphonooxy)methyl-2-oxo-2,3-dihydro-1,3-benzoxazol-5-yl]-2,4-pyrimidinediamine, or a pharmaceutically acceptable salt thereof, preferably N2-(3-methoxy-5-trifluoromethyl)phenyl-5-methyl-N4-[3-(phosphonooxy)methyl-2-oxo-2,3-dihydro-1,3-benzoxazol-5-yl]-2,4-pyrimidinediamine bis-sodium salt; or

5-(2-(3-methoxy-4,5-dimethylphenylamino)-5-methylpyrimidin-4-ylamino)benzo[d]oxazol-2(3H)-one, or a pharmaceutically acceptable salt thereof;

(5-(2-(4-fluoro-3-methoxy-5-methylphenylamino)-5-methylpyrimidin-4-ylamino)-2-oxobenzo[d]oxazol-3(2H)-yl)methyl dihydrogen phosphate;

5-(2-(4-fluoro-3-methoxy-5-methylphenylamino)-5-methylpyrimidin-4-ylamino)benzo[d]oxazol-2(3H)-one, or a pharmaceutically acceptable salt thereof; or

sodium (5-(2-(4-fluoro-3-methoxy-5-methylphenylamino)-5-methylpyrimidin-4-ylamino)-2-oxobenzo[d]oxazol-3(2H)-yl)methyl phosphate.

Additional information concerning the compounds of Formula I can be found in international publication No. WO/2010/085684 (International application No. PCT/US2010/021856) which is incorporated herein by reference in its entirety.

B. Pyrimidine Diamine Compounds according to Formula III

In some embodiments, the compound is a pyridine diamine compound according to Formula III

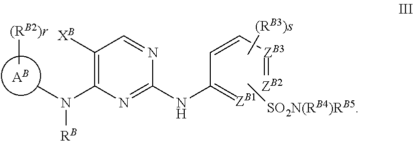

or a salt, solvate, N-oxide or prodrug thereof. With respect to Formula III, X^{B} is alkyl, alkoxy, amino, carboxyl, carboxyl ester, cyano, halo, nitro, alkenyl, or alkynyl, preferably halo, such as F;

R^{B} is hydrogen, alkyl, alkenyl, alkynyl, or cycloalkyl, preferable H;

ring A^{B} is aryl, heteroaryl, cycloalkyl, cycloalkenyl or heterocyclic, wherein ring A^{B} is not indolyl or benzimidazolyl, and in some embodiments, ring A^{B} is aryl, such as phenyl;

r is 0, 1, 2 or 3, and in certain embodiments, r is 1;

each R^{B2} independently is alkyl, alkoxy, amino, aryl, aryloxy (i.e. aryl-O—), cyano, cycloalkyl, cycloalkoxy, heteroaryl, heteroaryloxy, heterocyclic, heterocyclyloxy, aminoacyl, carboxyl, carboxyl ester, carbonate ester, sulfonyl, oxo, nitro or halo, preferably alkoxy, such as propynyloxy;

Z^{B1}, Z^{B2}, and Z^{B3} each independently is carbon or nitrogen, wherein if Z^{B1} is nitrogen then Z^{B2} and Z^{B3} are carbon, if Z^{B2} is nitrogen then Z^{B1} and Z^{B3} are carbon, and if Z^{B3} is nitrogen then Z^{B1} and Z^{B2} are carbon, wherein if Z^{B1}, Z^{B2}, or Z^{B3} is nitrogen then SO₂R^{B4}R^{B5} is not attached to the nitrogen, and preferably Z^{B1}, Z^{B2}, and Z^{B3} are carbon;

s is 0, 1, 2 or 3, preferably 3;

each R^{B3} independently is hydrogen, alkyl, alkoxy, or cycloalkyl, halo, or heterocyclic, preferably H or C₁₋₆alkyl, such as methyl;

each of R^{B4} and R^{B5} independently is hydrogen, alkyl, acyl or M⁺, wherein M⁺ is a metal counterion selected from K⁺, Na⁺, Li⁺ or ⁺N(R^{B6})4, wherein R^{B6} is hydrogen or alkyl, and the nitrogen of SO₂NR^{B4}R^{B5} is N⁻; or R^{B4} or R^{B5} is a divalent counterion selected from Ca²⁺, Mg²+, and Ba²⁺, and the nitrogen of SO₂NR^{B4}R^{B5} is and in some embodiments, each of R^{B4} and R^{B5} independently is hydrogen, alkyl, or acyl, such as H or acyl, and in certain embodiments, one or R^{B4} and R^{B5} is H and the other is H or acyl, such as propionyl.

In some embodiments:

if r=0, then X^{B} is not bromo;

if ring A^{B} is cycloalkyl, then X^{B} is not bromo;

if r=2 and each of R^{B2} is methoxy, halo, trihalomethyl or trihalomethoxy, then R^{B4} and R^{B5} are not one hydrogen and one methyl;

if r=2 and R^{B2} is fluoro and methyl, then R^{B} is not alkenyl; and

if ring A^{B} is phenyl, r=1 and R^{B2} is chloro, then R^{B4} and R^{B5} are not one hydrogen and one methyl.

Exemplary compounds according to Formula III include, but are not limited to

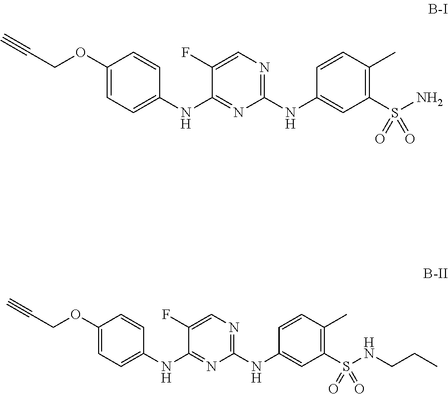 [SMILES: C#CCOc1ccc(Nc2nc(Nc3ccc(C)c(S(N)(=O)=O)c3)ncc2F)cc1 ; C#CCOc1ccc(Nc2nc(Nc3ccc(C)c(S(=O)(=O)NCCC)c3)ncc2F)cc1]

or a pharmaceutically acceptable salt, solvate, N-oxide or prodrug thereof. Compound B-I is also referred to as N2-(3-amino sulfonyl-4-methylphenyl)-5-fluoro-N4-[4-(prop-2-ynyloxy)phenyl]-2,4-pyrimidinediamine Compound B-II is also referred to as 5-fluoro-N2-(4-methyl-3-propionylaminosulfonylphenyl)-N4-[4-(prop-2-ynyloxy)phenyl]-2,4-pyrimidinediamine.

One of ordinary skill in the art will appreciate that compound B-II maybe a prodrug of compound B-I, and that compound B-II need not necessarily be, pharmacologically inactive until converted into compound B-I. The mechanism by which the propionyl progroup metabolizes is not critical, and can be caused by, for example, hydrolysis under the acidic conditions of the stomach, and/or by enzymes present in the digestive tract and/or tissues or organs of the body, for example, esterases, amidases, lipolases, phosphatases including ATPases and kinases, cytochrome P450's of the liver, and the like.

Additional information concerning compounds according to Formula III, such as compounds B-I and B-II, can be found in international publication Nos. WO2011/017178 (international application No. PCT/US2010/043592) and WO2006/133426 (international application No. PCT/US2006/022590), both of which are incorporated herein by reference in their entireties.

C. Pyrazole Compounds

In some embodiments, the compound is a pyrazole compound. The compound may have a formula

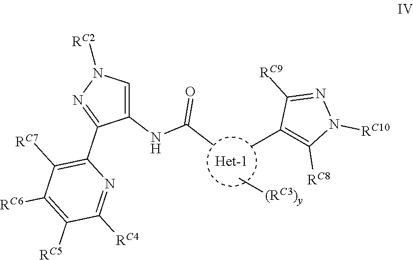

or a salt, prodrug, solvate and/or N-oxide thereof. With respect to Formula IV, Het-1 is 5-membered heteroaryl, such as thiazolyl or furanyl;

y is from 1to 2;

R^{C2} is H, aliphatic, heteroaliphatic, heterocycloaliphatic, aryl, amide, heterocyclyl or araliphatic, such as H alkyl, haloalkyl or cycloalkyl, and in some embodiments, R^{C2} is alkyl, haloaklyl, or cycloalkyl;

each R^{C3} independently is H or aliphatic, such as H or alkyl;

R^{C4}, R^{C5}, R^{C6} and R^{C7} are each independently H, aliphatic, heteroaliphatic, alkoxy, heterocyclyl, aryl, araliphatic, —O-heterocyclyl, hydroxyl, haloalkyl, halogen, nitro, cyano, carboxyl, carboxyl ester, acyl, amide, amino, sulfonyl, sulfonamide, sulfanyl or sulfinyl;

R^{C8} and R^{C9} are each independently H, aliphatic, heteroaliphatic, aryl, heterocyclyl, sulfonyl, nitro, halogen, haloalkyl, carboxyl ester, cyano or amino, such as H, halogen, haloalkyl, or alkyl, and in some embodiments, each of R^{C8} and R^{C9} is independently H or aliphatic, such as H, alkyl or haloalkyl.

R^{C10} is H, aliphatic, alkoxy, heteroaliphatic, carboxyl ester, araliphatic, NO₂, CN, OH, haloalkyl, acyl, alkyl phosphate or alkylphosphonate, such as H, aliphatic such as alkyl, carboxyl ester, acyl, alkyl phosphate, alkyl phosphonate or aralkyl, and in some embodiments, R^{C10} is H, alkyl, alkyl phosphate or alkyl phosphonate.

In some embodiments, each of R^{C4}, R^{C6}, and R^{C7} independently is H; halo, such as F; or aliphatic, such as alkyl or haloalkyl, preferably CF₃, and/or R^{C5} is H; halo, such as F; aliphatic, such as alkyl or haloalkyl, preferably CF₃; alkoxy, such as methoxy or —O—CH₂C(CH₃)₂OH; heterocyclyl, such as morpholin-4-yl or 1-methylpiperidin-4-yl; or —O-heterocyclyl, such as —O-(oxetan-3-yl). In particular embodiments, each of R^{C4}, R^{C5}, R^{C6} and R^{C7} independently are H or F. And in certain embodiments, at least one of R^{C4}, R^{C5}, R^{C6} and R^{C7} is not H.

In some embodiments, the compound has a formula V or VI

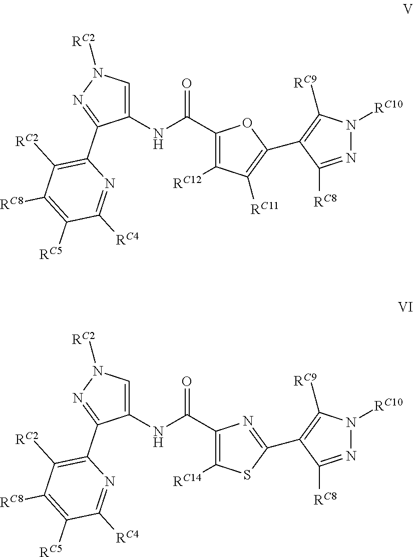

or a salt, prodrug, solvate and/or N-oxide thereof. With respect to Formula V and Formula VI, the variables are as previously defined for Formula IV, and each of R^{C11}, R^{C12} and R^{C14} independently is H or aliphatic, such as H or alkyl.

Exemplary compounds according to Formula IV include, but are not limited to, those listed below in List 2.

### List 2: Exemplary Compounds According to Formula IV

V-1: N-(1-(2-hydroxy-2-methylpropyl)-3-(pyridin-2-yl)-1H-pyrazol-4-yl)-5-(1-methyl-1H-pyrazol-4-yl)furan-2-carboxamide 2,2,2-trifluoroacetate;

V-2: N-(1-(2-hydroxy-2-methylpropyl)-3-(pyridin-2-yl)-1H-pyrazol-4-yl)-5-(1-methyl-1H-pyrazol-4-yl)furan-2-carboxamide;

V-3: N-(1-(2-hydroxy-2-methylpropyl)-3-(pyridin-2-yl)-1H-pyrazol-4-yl)-5-(1H-pyrazol-4-yl)furan-2-carboxamide;

V-4: tert-butyl 4-(5-((1-(2-ethoxyethyl)-3-(pyridin-2-yl)-1H-pyrazol-4-yl)carbamoyl)furan-2-yl)-1H-pyrazole-1-carboxylate;

V-5: N-(1-(2-methoxyethyl)-3-(pyridin-2-yl)-1H-pyrazol-4-yl)-5-(1H-pyrazol-4-yl)furan-2-carboxamide;

V-6: N-(1-(2-methoxyethyl)-3-(pyridin-2-yl)-1H-pyrazol-4-yl)-5-(3-methyl-1H-pyrazol-4-yl)furan-2-carboxamide formic acid;

V-9: N-(1-(2-methoxyethyl)-3-(pyridin-2-yl)-1H-pyrazol-4-yl)-5-(3-methyl-1H-pyrazol-4-yl)furan-2-carboxamide;

V-10: di-tert-butyl ((4-(5-((1-(2-methoxyethyl)-3-(pyridin-2-yl)-1H-pyrazol-4-yl)carbamoyl)furan-2-yl)-1H-pyrazol-1-yl)methyl) phosphate;

V-11: tert-butyl ((4-(5-((1-(2-methoxyethyl)-3-(pyridin-2-yl)-1H-pyrazol-4-yl)carbamoyl)furan-2-yl)-1H-pyrazol-1-yl)methyl) hydrogen phosphate;

V-12: (4-(5-((1-(2-methoxyethyl)-3-(pyridin-2-yl)-1H-pyrazol-4-yl)carbamoyl)furan-2-yl)-1H-pyrazol-1-yl)methyl dihydrogen phosphate;

V-13: N-(1-(2-methoxyethyl)-3-(pyridin-2-yl)-1H-pyrazol-4-yl)-5-(3-(trifluoromethyl)-1H-pyrazol-4-yl)furan-2-carboxamide;

V-14: sodium (4-(5-((1-(2-methoxyethyl)-3-(pyridin-2-yl)-1H-pyrazol-4-yl)carbamoyl)furan-2-yl)-1H-pyrazol-1-yl)methyl phosphate;

V-16: N-(1-(2-(2-methoxyethoxy)ethyl)-3-(pyridin-2-yl)-1H-pyrazol-4-yl)-5-(1H-pyrazol-4-yl)furan-2-carboxamide;

V-17: N-(1-(2-(2-methoxyethoxy)ethyl)-3-(pyridin-2-yl)-1H-pyrazol-4-yl)-5-(1H-pyrazol-4-yl)furan-2-carboxamide hydrochloride salt;

V-18: N-(1-(2-(2-methoxyethoxy)ethyl)-3-(pyridin-2-yl)-1H-pyrazol-4-yl)-5-(3-methyl-1H-pyrazol-4-yl)furan-2-carboxamide;

V-19: 1-(isobutyryloxy)ethyl 4-(5-((1-(2-methoxyethyl)-3-(pyridin-2-yl)-1H-pyrazol-4-yl)carbamoyl)furan-2-yl)-1H-pyrazole-1-carboxylate;

V-20: tert-butyl (S)—(1-(4-(5-((1-(2-methoxyethyl)-3-(pyridin-2-yl)-1H-pyrazol-4-yl)carbamoyl)furan-2-yl)-1H-pyrazol-1-yl)-3-methyl-1-oxobutan-2-yl)carbamate;

V-21: 1-methylcyclopropyl 4-(5-((1-(2-methoxyethyl)-3-(pyridin-2-yl)-1H-pyrazol-4-yl)carbamoyl)furan-2-yl)-1H-pyrazole-1-carboxylate;

V-22: 1-((4-methoxybenzypoxy)-2-methylpropan-2-yl 4-(5-((1-(2-methoxyethyl)-3-(pyridin-2-yl)-1H-pyrazol-4-yl)carbamoyl)furan-2-yl)-1H-pyrazole-1-carboxylate;

V-23: 5-(1H-pyrazol-4-yl)-N-(3-(pyridin-2-yl)-1-(tetrahydro-2H-pyran-4-yl)-1H-pyrazol-4-yl)furan-2-carboxamide;

V-24: 5-(5-nitro-1H-pyrrol-3-yl)-N-(1-(propoxymethyl)-3-(pyridin-2-yl)-1H-pyrazol-4-yl)furan-2-carboxamide;

V-25: N-(1-(oxetan-3-yl)-3-(pyridin-2-yl)-1H-pyrazol-4-yl)-5-(1H-pyrazol-4-yl)furan-2-carboxamide;

V-26: 5-(1-methyl-1H-pyrazol-4-yl)-N-(1-(oxetan-3-yl)-3-(pyridin-2-yl)-1H-pyrazol-4-yl)furan-2-carboxamide;

V-27: N-(1-((1,3-trans)-3-ethoxycyclobutyl)-3-(pyridin-2-yl)-1H-pyrazol-4-yl)-5-(1H-pyrazol-4-yl)furan-2-carboxamide;

V-28: N-(1-((1,3-cis)-3-ethoxycyclobutyl)-3-(pyridin-2-yl)-1H-pyrazol-4-yl)-5-(1H-pyrazol-4-yl)furan-2-carboxamide;

V-29: N-(1-((1,3-cis)-3-ethoxycyclobutyl)-3-(pyridin-2-yl)-1H-pyrazol-4-yl)-5-(3-methyl-1H-pyrazol-4-yl)furan-2-carboxamide;

V-30: 5-(3-methyl-1H-pyrazol-4-yl)-N-(1-(oxetan-3-yl)-3-(pyridin-2-yl)-1H-pyrazol-4-yl)furan-2-carboxamide;

V-31: N-(1-((1,3-cis)-3-ethoxycyclobutyl)-3-(pyridin-2-yl)-1H-pyrazol-4-yl)-5-(1-methyl-1H-pyrazol-4-yl)furan-2-carboxamide;

V-32: N-(1-((1,3-cis)-3-hydroxycyclobutyl)-3-(pyridin-2-yl)-1H-pyrazol-4-yl)-5-(1H-pyrazol-4-yl)furan-2-carboxamide;

V-33: N-(1-((1s,3s)-3-(dimethylamino)cyclobutyl)-3-(pyridin-2-yl)-1H-pyrazol-4-yl)-5-(1H-pyrazol-4-yl)furan-2-carboxamide;

V-34: N-(1-((1s,3s)-3-(dimethylamino)cyclobutyl)-3-(pyridin-2-yl)-1H-pyrazol-4-yl)-5-(1H-pyrazol-4-yl)furan-2-carboxamide formate;

V-35: (4-(5-((1-((1,3-cis)-3-ethoxycyclobutyl)-3-(pyridin-2-yl)-1H-pyrazol-4-yl)carbamoyl)furan-2-yl)-1H-pyrazol-1-yl)methyl phosphate bis-sodium salt;

V-36: (4-(5-((1-((1s,3s)-3-ethoxycyclobutyl)-3-(pyridin-2-yl)-1H-pyrazol-4-yl)carbamoyl)furan-2-yl)-1H-pyrazol-1-yl)methyl dihydrogen phosphate;

V-37: N-(1-(2-ethoxyethyl)-3-(pyridin-2-yl)-1H-pyrazol-4-yl)-5-(1H-pyrazol-4-yl)furan-2-carboxamide formate;

V-38: N-(1-(2-ethoxyethyl)-3-(pyridin-2-yl)-1H-pyrazol-4-yl)-5-(1H-pyrazol-4-yl)furan-2-carboxamide;

V-39: N-(1-(2-ethoxyethyl)-3-(pyridin-2-yl)-1H-pyrazol-4-yl)-5-(1-ethyl-1H-pyrazol-4-yl)furan-2-carboxamide formate;

V-40: N-(1-(2-ethoxyethyl)-3-(pyridin-2-yl)-1H-pyrazol-4-yl)-5-(1-ethyl-1H-pyrazol-4-yl)furan-2-carboxamide;

V-41: N-(1-(2-ethoxyethyl)-3-(pyridin-2-yl)-1H-pyrazol-4-yl)-5-(3-(trifluoromethyl)-1H-pyrazol-4-yl)furan-2-carboxamide formate;

V-42: N-(1-(2-ethoxyethyl)-3-(pyridin-2-yl)-1H-pyrazol-4-yl)-5-(3-(trifluoromethyl)-1H-pyrazol-4-yl)furan-2-carboxamide;

V-43: N-(1-(2-ethoxyethyl)-3-(pyridin-2-yl)-1H-pyrazol-4-yl)-5-(1-isopentyl-1H-pyrazol-4-yl)furan-2-carboxamide formate;

V-44: N-(1-(2-ethoxyethyl)-3-(pyridin-2-yl)-1H-pyrazol-4-yl)-5-(1-isopentyl-1H-pyrazol-4-yl)furan-2-carboxamide;

V-45: N-(1-(2-ethoxyethyl)-3-(pyridin-2-yl)-1H-pyrazol-4-yl)-5-(1-methyl-1H-pyrazol-4-yl)furan-2-carboxamide formate;

V-46: N-(1-(2-ethoxyethyl)-3-(pyridin-2-yl)-1H-pyrazol-4-yl)-5-(1-methyl-1H-pyrazol-4-yl)furan-2-carboxamide;

V-47: 5-(1H-pyrazol-4-yl)-N-(3-(pyridin-2-yl)-1H-pyrazol-4-yl)furan-2-carboxamide;

V-48: 5-(1-((3-methyloxetan-3-yl)methyl)-1H-pyrazol-4-yl)-N-(1-((3-methyloxetan-3-yl)methyl)-3-(pyridin-2-yl)-1H-pyrazol-4-yl)furan-2-carboxamide formate;

V-49: 5-(1-((3-methyloxetan-3-yl)methyl)-1H-pyrazol-4-yl)-N-(1-((3-methyloxetan-3-yl)methyl)-3-(pyridin-2-yl)-1H-pyrazol-4-yl)furan-2-carboxamide;

V-52: 5-(1-(2-(2-methoxyethoxy)ethyl)-1H-pyrazol-4-yl)-N-(1-(2-(2-methoxyethoxy)ethyl)-3-(pyridin-2-yl)-1H-pyrazol-4-yl)furan-2-carboxamide formate;

V-53: 5-(1-(2-(2-methoxyethoxy)ethyl)-1H-pyrazol-4-yl)-N-(1-(2-(2-methoxyethoxy)ethyl)-3-(pyridin-2-yl)-1H-pyrazol-4-yl)furan-2-carboxamide;

V-54: (4-(5-((1-(2-ethoxyethyl)-3-(pyridin-2-yl)-1H-pyrazol-4-yl)carbamoyl)furan-2-yl)-1H-pyrazol-1-yl)methyl dihydrogen phosphate;

V-55: sodium (4-(5-((1-(2-ethoxyethyl)-3-(pyridin-2-yl)-1H-pyrazol-4-yl)carbamoyl)furan-2-yl)-1H-pyrazol-1-yl)methyl phosphate;

V-56: N-(1-(2-ethoxyethyl)-3-(pyridin-2-yl)-1H-pyrazol-4-yl)-5-(3-methyl-1H-pyrazol-4-yl)furan-2-carboxamide formate;

V-57: N-(1-(2-ethoxyethyl)-3-(pyridin-2-yl)-1H-pyrazol-4-yl)-5-(3-methyl-1H-pyrazol-4-yl)furan-2-carboxamide;

V-58: 5-(3,5-dimethyl-1H-pyrazol-4-yl)-N-(1-(2-ethoxyethyl)-3-(pyridin-2-yl)-1H-pyrazol-4-yl)furan-2-carboxamide formate;

V-59: 5-(3,5-dimethyl-1H-pyrazol-4-yl)-N-(1-(2-ethoxyethyl)-3-(pyridin-2-yl)-1H-pyrazol-4-yl)furan-2-carboxamide;

V-67: N-{1-Methyl-3-(pyridine-2-yl)-1H-pyrazol-4-yl}-5-(1H-pyrazol-4-yl)furan-2-carboxamide, formate salt;

V-68: 5-(1-Methyl-1H-pyrazol-4-yl)-N-{1-methyl-3-(pyridine-2-yl)-1H-pyrazol-4-yl}furan-2-carboxamide;

V-69: 5-(1-Methyl-1H-pyrazol-4-yl)-N-{1-methyl-3-(pyridine-2-yl)-1H-pyrazol-4-yl}furan-2-carboxamide, formate salt;

V-70: tert-Butyl-3-[4-{5-(1H-pyrazole-4-yl)furan-2-carboxamido}-3-(pyridine-2-yl)-1H-pyrazol-1-yl]azetidine-1-carboxylate, formate salt;

V-71: N-{1-(3-Methoxycyclobutyl)-3-(pyridine-2-yl)-1H-pyrazol-4-yl}-5-(1H-pyrazol-4-yl)furan-2-carboxamide, formate salt, Cis isomer;

V-72: N-{1-(3-Methoxycyclobutyl)-3-(pyridine-2-yl)-1H-pyrazol-4-yl}-5-(1H-pyrazol-4-yl)furan-2-carboxamide, Cis isomer;

V-73: N-{1-(3-Benzyloxy)cyclobutyl)-3-(pyridine-2-yl)-1H-pyrazol-4-yl}-5-(1H-pyrazol-4-yl)furan-2-carboxamide, Trans isomer;

V-74: tert-Butyl-3-[4-{5-(1H-pyrazole-4-yl)furan-2-carboxamido}-3-(pyridine-2-yl)-1H-pyrazol-1-yl]azetidine-1-carboxylate;

V-75: N-(1-((1s,3s)-3-methoxycyclobutyl)-3-(pyridin-2-yl)-1H-pyrazol-4-yl)-5-(1H-pyrazol-4-yl)furan-2-carboxamide formate;

V-76: N-(1-((1s,3s)-3-methoxycyclobutyl)-3-(pyridin-2-yl)-1H-pyrazol-4-yl)-5-(1H-pyrazol-4-yl)furan-2-carboxamide;

V-77: N-{1-Methyl-3-(pyridine-2-yl)-1H-pyrazol-4-yl}-5-(1H-pyrazol-4-yl)furan-2-carboxamide, free base;

V-78: N-{1-(Azetidin-3-yl)-3-(pyridine-2-yl)-1H-pyrazol-4-yl}-5-(1H-pyrazol-4-yl)furan-2-carboxamide, TFA salt;

V-79: N-{1-(Azetidin-3-yl)-3-(pyridine-2-yl)-1H-pyrazol-4-yl}-5-(1H-pyrazol-4-yl)furan-2-carboxamide;

V-80: Di-tert-butyl-[[4-{4-(5-((1-methyl-3-(pyridine-2-yl)-1H-pyrazol-4-yl)carbamoyl)furan-2-yl)-1H-pyrazol-1-yl}methyl]phosphate;

V-81: [4-{5-((1-Methyl-3-(pyridine-2-yl)-1H-pyrazol-4-yl)carbamoyl)furan-2yl}-1H-pyrazol-1-yl]methyl dihydrogen phosphate;

V-82: Sodium [4-{5-((1-Methyl-3-(pyridine-2-yl)-1H-pyrazol-4-yl)carbamoyl)furan-2-yl}-1H-pyrazol-1-yl]methyl phosphate;

V-83: N-{1-(1-Acetylazetidin-3-yl)-3-(pyridine-2-yl)-1H-pyrazol-4-yl}-5-(1H-pyrazol-4-yl)furan-2-carboxamide, free base;

V-84: 3-[4-{5-(1H-Pyrazol-4-yl)furan-2-carboxamido}-3-(pyridine-2-yl)-1H-pyrazol-1-yl]-N-(tert-butypazetidine-1-carboxamide, free base;

V-85: 3-[4-{5-(1H-Pyrazol-4-yl)furan-2-carboxamido}-3-(pyridine-2-yl)-1H-pyrazol-1-yl]-N-isopropylazetidine-1-carboxamide, free base;

V-86: 3-[4-{5-(1H-Pyrazol-4-yl)furan-2-carboxamido}-3-(pyridine-2-yl)-1H-pyrazol-1-yl]-N-propylazetidine-1-carboxamide, free base.

V-87: 3-[4-{5-(1H-Pyrazol-4-yl)furan-2-carboxamido}-3-(pyridine-2-yl)-1H-pyrazol-1-yl]-N-cyclopropylazetidine-1-carboxamide, formate salt;

V-88: 3-[4-{5-(1H-Pyrazol-4-yl)furan-2-carboxamido}-3-(pyridine-2-yl)-1H-pyrazol-1-yl]-N-cyclopropylazetidine-1-carboxamide;

V-89: N-[1-{1-(Cyclopropanecarbonyl)azetidin-3-yl}-3-(pyridine-2-yl)-1H-pyrazol-4-yl]-5-(1H-pyrazol-4-yl)furan-2-carboxamide, formate salt;

V-90: N-[1-{1-(Cyclopropanecarbonyl)azetidin-3-yl}-3-(pyridine-2-yl)-1H-pyrazol-4-yl]-5-(1H-pyrazol-4-yl)furan-2-carboxamide;

V-91: N-[1-{1-Pivaloylazetidin-3-yl}-3-(pyridine-2-yl)-1H-pyrazol-4-yl]-5-(1H-pyrazol-4-yl)furan-2-carboxamide, formate salt;

V-92: N-[1-{1-Pivaloylazetidin-3-yl}-3-(pyridine-2-yl)-1H-pyrazol-4-yl]-5-(1H-pyrazol-4-yl)furan-2-carboxamide;

V-93: 5-(1H-Pyrazol-4-yl)-N-{3-(pyridine-2-yl)-1-(pyrrolidine-1-carbonyl)azetidin-3-yl}-1H-pyrazol-4-yl)furan-2-carboxamide, formate salt;

V-94: 5-(1H-Pyrazol-4-yl)-N-{3-(pyridine-2-yl)-1-(pyrrolidine-1-carbonyl)azetidin-3-yl}-1H-pyrazol-4-yl)furan-2-carboxamide;

V-95: N-[1-{1-Isobutyrylazetidin-3-yl}-3-(pyridine-2-yl)-1H-pyrazol-4-yl]-5-(1H-pyrazol-4-yl)furan-2-carboxamide, formate salt;

V-96: N-[1-{1-Isobutyrylazetidin-3-yl}-3-(pyridine-2-yl)-1H-pyrazol-4-yl]-5-(1H-pyrazol-4-yl)furan-2-carboxamide;

V-97: N-(1H-Pyrazol-4-yl)-N-{3-(pyridine-2-yl)-1-{1-(2,2,2-trifluoroethypazetidin-3-yl}-1H-pyrazol-4-yl}furan-2-carboxamide, TFA salt;

V-98: N-(1H-Pyrazol-4-yl)-N-{3-(pyridine-2-yl)-1-{1-(2,2,2-trifluoroethypazetidin-3-yl}-1H-pyrazol-4-yl}furan-2-carboxamide;

V-99: N-[1-{1-Butyrylazetidin-3-yl}-3-(pyridine-2-yl)-1H-pyrazol-4-yl]-5-(1H-pyrazol-4--yl)furan-2-carboxamide, formate salt;

V-100: N-[1-{1-Butyrylazetidin-3-yl}-3-(pyridine-2-yl)-1H-pyrazol-4-yl]-5-(1H-pyrazol-4-yl)furan-2-carboxamide;

V-101: N-{-(1-Methylazetidin-3-yl)-3-(pyridine-2-yl)-1H-pyrazol-4-yl}-5-(1H-pyrazol-4-yl)furan-2-carboxamide, formate salt;

V-102: N-{-(1-Methylazetidin-3-yl)-3-(pyridine-2-yl)-1H-pyrazol-4-yl}-5-(1H-pyrazol-4-yl)furan-2-carboxamide;

V-103: N-[1-{1-(2,2-difluorocyclopropane-1-carbonyl)azetidin-3-yl}-3-(pyridine-2-yl)-1H-pyrazol-4-yl]-5-(1H-pyrazol-4-yl)furan-2-carboxamide, formate salt;

V-104: N-[1-{1-(2,2-difluorocyclopropane-1-carbonyl)azetidin-3-yl}-3-(pyridine-2-yl)-1H-pyrazol-4-yl]-5-(1H-pyrazol-4-yl)furan-2-carboxamide;

V-105: N-(1-methyl-3-(5-morpholinopyridin-2-yl)-1H-pyrazol-4-yl)-5-(1H-pyrazol-4-yl)furan-2-carboxamide;

V-106: N-(1-methyl-3-(5-(4-methylpiperazin-1-yl)pyridin-2-yl)-1H-pyrazol-4-yl)-5-(1H-pyrazol-4-yl)furan-2-carboxamide;

V-107: N-(3-(5-(2-hydroxy-2-methylpropoxy)pyridin-2-yl)-1-methyl-1H-pyrazol-4-yl)-5-(1H-pyrazol-4-yl)furan-2-carboxamide;

V-108: N-(1-methyl-3-(5-(oxetan-3-yloxy)pyridin-2-yl)-1H-pyrazol-4-yl)-5-(1H-pyrazol-4-yl)furan-2-carboxamide;

V-109: N-(3-(5-methoxypyridin-2-yl)-1-methyl-1H-pyrazol-4-yl)-5-(1H-pyrazol-4-yl)furan-2-carboxamide;

V-110: N-(1-isopropyl-3-(pyridin-2-yl)-1H-pyrazol-4-yl)-5-(1H-pyrazol-4-yl)furan-2-carboxamide;

V-111: N-(1-(2-morpholinoethyl)-3-(pyridin-2-yl)-1H-pyrazol-4-yl)-5-(1H-pyrazol-4-yl)furan-2-carboxamide;

V-112: N-(1-(2-(4-methylpiperazin-1-yl)ethyl)-3-(pyridin-2-yl)-1H-pyrazol-4-yl)-5-(1H-pyrazol-4-yl)furan-2-carboxamide;

V-113: 5-(1H-pyrazol-3-yl)-N-(3-(pyridin-2-yl)-1-(2-(2,2,2-trifluoroethoxy)ethyl)-1H-pyrazol-4-yl)furan-2-carboxamide;

V-114: N-(1-((1s,3s)-3-isopropoxycyclobutyl)-3-(pyridin-2-yl)-1H-pyrazol-4-yl)-5-(1H-pyrazol-4-yl)furan-2-carboxamide;

V-115: N-(1-(difluoromethyl)-3-(pyridin-2-yl)-1H-pyrazol-4-yl)-5-(1H-pyrazol-4-yl)furan-2-carboxamide;

V-116: N-(1-((1s,3s)-3-ethoxycyclobutyl)-3-(6-(trifluoromethyppyridin-2-yl)-1H-pyrazol-4-yl)-5-(1H-pyrazol-4-yl)furan-2-carboxamide;

V-117: 5-(1H-pyrazol-4-yl)-N-(3-(pyridin-2-yl)-1-(2,2,2-trifluoroethyl)-1H-pyrazol-4-yl)furan-2-carboxamide;

V-122: 5-(1-cyclobutyl-1H-pyrazol-4-yl)-N-(1-cyclobutyl-3-(pyridin-2-yl)-1H-pyrazol-4-yl)furan-2-carboxamide 2,2,2-trifluoroacetate;

V-123: 5-(1-cyclobutyl-1H-pyrazol-4-yl)-N-(1-cyclobutyl-3-(pyridin-2-yl)-1H-pyrazol-4-yl)furan-2-carboxamide;

V-124: N-(1-((1s,4s)-4-hydroxycyclohexyl)-3-(pyridin-2-yl)-1H-pyrazol-4-yl)-5-(1H-pyrazol-4-yl)furan-2-carboxamide formate;

V-125: N-(1-((1s,4s)-4-hydroxycyclohexyl)-3-(pyridin-2-yl)-1H-pyrazol-4-yl)-5-(1H-pyrazol-4-yl)furan-2-carboxamide;

V-126: N-(1-((1r,4r)-4-hydroxycyclohexyl)-3-(pyridin-2-yl)-1H-pyrazol-4-yl)-5-(1H-pyrazol-4-yl)furan-2-carboxamide formate;

V-127: N-(1-((1r,4r)-4-hydroxycyclohexyl)-3-(pyridin-2-yl)-1H-pyrazol-4-yl)-5-(1H-pyrazol-4-yl)furan-2-carboxamide;

V-128: 5-(1H-pyrazol-4-yl)-N-(3-(pyridin-2-yl)-1-(3-(2,2,2-trifluoroethoxy)cyclobutyl)-1H-pyrazol-4-yl)furan-2-carboxamide formate;

V-129: 5-(1H-pyrazol-4-yl)-N-(3-(pyridin-2-yl)-1-(3-(2,2,2-trifluoroethoxy)cyclobutyl)-1H-pyrazol-4-yl)furan-2-carboxamide;

V-130: N-(1-((1r,4r)-4-ethoxycyclohexyl)-3-(pyridin-2-yl)-1H-pyrazol-4-yl)-5-(1H-pyrazol-4-yl)furan-2-carboxamide formate;

V-131: N-(1-((1r,4r)-4-ethoxycyclohexyl)-3-(pyridin-2-yl)-1H-pyrazol-4-yl)-5-(1H-pyrazol-4-yl)furan-2-carboxamide;

V-132: N-(1-((1 S,3R)-3-ethoxycyclopentyl)-3-(pyridin-2-yl)-1H-pyrazol-4-yl)-5-(1H-pyrazol-4-yl)furan-2-carboxamide formate;

V-133: N-(1-((1S,3R)-3-ethoxycyclopentyl)-3-(pyridin-2-yl)-1H-pyrazol-4-yl)-5-(1H-pyrazol-4-yl)furan-2-carboxamide;

V-134: N-(1-((1S,3R)-3-hydroxycyclopentyl)-3-(pyridin-2-yl)-1H-pyrazol-4-yl)-5-(1H-pyrazol-4-yl)furan-2-carboxamide formate;

V-135: N-(1-((1S,3R)-3-hydroxycyclopentyl)-3-(pyridin-2-yl)-1H-pyrazol-4-yl)-5-(1H-pyrazol-4-yl)furan-2-carboxamide;

V-136: N-(1-((1S,3S)-3-hydroxycyclopentyl)-3-(pyridin-2-yl)-1H-pyrazol-4-yl)-5-(1H-pyrazol-4-yl)furan-2-carboxamide formate;

V-137: N-(1-((1S,3S)-3-hydroxycyclopentyl)-3-(pyridin-2-yl)-1H-pyrazol-4-yl)-5-(1H-pyrazol-4-yl)furan-2-carboxamide;

V-138: N-(1-((1s,3 s)-3-ethoxycyclobutyl)-3-(5-fluoropyridin-2-yl)-1H-pyrazol-4-yl)-5-(1H-pyrazol-4-yl)furan-2-carboxamide formate;

V-139: N-(1-((1s,3 s)-3-ethoxycyclobutyl)-3-(5-fluoropyridin-2-yl)-1H-pyrazol-4-yl)-5-(1H-pyrazol-4-yl)furan-2-carboxamide;

V-140: N-(1-((1S,3R)-3-ethoxy-2-fluorocyclobutyl)-3-(pyridin-2-yl)-1H-pyrazol-4-yl)-5-(1H-pyrazol-4-yl)furan-2-carboxamide formate;

V-141: N-(1-((1S,3R)-3-ethoxy-2-fluorocyclobutyl)-3-(pyridin-2-yl)-1H-pyrazol-4-yl)-5-(1H-pyrazol-4-yl)furan-2-carboxamide;

V-142: N-(1-((1s,3 s)-3-ethoxycyclobutyl)-3-(3-fluoropyridin-2-yl)-1H-pyrazol-4-yl)-5-(1H-pyrazol-4-yl)furan-2-carboxamide formate;

V-143: N-(1-((1s,3 s)-3-ethoxycyclobutyl)-3-(3-fluoropyridin-2-yl)-1H-pyrazol-4-yl)-5-(1H-pyrazol-4-yl)furan-2-carboxamide;

V-144: N-(1-((1s,3s)-3-ethoxycyclobutyl)-3-(6-fluoropyridin-2-yl)-1H-pyrazol-4-yl)-5-(1H-pyrazol-4-yl)furan-2-carboxamide formate;

V-145: N-(1-((1s,3s)-3-ethoxycyclobutyl)-3-(6-fluoropyridin-2-yl)-1H-pyrazol-4-yl)-5-(1H-pyrazol-4-yl)furan-2-carboxamide;

V-146: 5-(1H-pyrazol-4-yl)-N-(3-(pyridin-2-yl)-1-((1s,3 s)-3-(2,2,2-trifluoroethoxy)cyclobutyl)-1H-pyrazol-4-yl)furan-2-carboxamide formate;

V-147: 5-(1H-pyrazol-4-yl)-N-(3-(pyridin-2-yl)-1-((1s,3 s)-3-(2,2,2-trifluoroethoxy)cyclobutyl)-1H-pyrazol-4-yl)furan-2-carboxamide;

V-148: N-(1-((1s,3s)-3-ethoxycyclobutyl)-3-(4-fluoropyridin-2-yl)-1H-pyrazol-4-yl)-5-(1H-pyrazol-4-yl)furan-2-carboxamide formate;

V-149: N-(1-((1s,3s)-3-ethoxycyclobutyl)-3-(4-fluoropyridin-2-yl)-1H-pyrazol-4-yl)-5-(1H-pyrazol-4-yl)furan-2-carboxamide;

V-150: N-(3-(6-fluoropyridin-2-yl)-1-((1s,3 s)-3-(2,2,2-trifluoroethoxy)cyclobutyl)-1H-pyrazol-4-yl)-5-(1H-pyrazol-4-yl)furan-2-carboxamide formate;

V-151: N-(3-(6-fluoropyridin-2-yl)-1-((1s,3 s)-3-(2,2,2-trifluoroethoxy)cyclobutyl)-1H-pyrazol-4-yl)-5-(1H-pyrazol-4-yl)furan-2-carboxamide;

V-152: N-(3-(3-fluoropyridin-2-yl)-1-((1s,3 s)-3-(2,2,2-trifluoroethoxy)cyclobutyl)-1H-pyrazol-4-yl)-5-(1H-pyrazol-4-yl)furan-2-carboxamide formate;

V-153: N-(3-(3-fluoropyridin-2-yl)-1-((1s,3 s)-3-(2,2,2-trifluoroethoxy)cyclobutyl)-1H-pyrazol-4-yl)-5-(1H-pyrazol-4-yl)furan-2-carboxamide;

V-154: N-(1-((1r,4r)-4-ethoxycyclohexyl)-3-(3-fluoropyridin-2-yl)-1H-pyrazol-4-yl)-5-(1H-pyrazol-4-yl)furan-2-carboxamide formate;

V-155: N-(1-((1r,4r)-4-ethoxycyclohexyl)-3-(3-fluoropyridin-2-yl)-1H-pyrazol-4-yl)-5-(1H-pyrazol-4-yl)furan-2-carboxamide;

V-156: N-(3-(3,6-difluoropyridin-2-yl)-1-((1s,3 s)-3-ethoxycyclobutyl)-1H-pyrazol-4-yl)-5-(1H-pyrazol-4-yl)furan-2-carboxamide;

VI-1: N-(1-(2-hydroxy-2-methylpropyl)-3-(pyridin-2-yl)-1H-pyrazol-4-yl)-2-(1H-pyrazol-4-yl)thiazole-4-carboxamide;

VI-2: 1-(isobutyryloxy)ethyl 4-(4-((1-methyl-3-(pyridin-2-yl)-1H-pyrazol-4-yl)carbamoyl)thiazol-2-yl)-1H-pyrazole-1-carboxylate;

VI-3: tert-butyl (R)—(3-methyl-1-(4-(4-((1-methyl-3-(pyridin-2-yl)-1H-pyrazol-4-yl)carbamoyl)thiazol-2-yl)-1H-pyrazol-1-yl)-1-oxobutan-2-yl)carbamate;

VI-4: 2-(1-((5-methyl-2-oxo-1,3-dioxol-4-yl)methyl)-1H-pyrazol-4-yl)-N-(1-methyl-3-(pyridin-2-yl)-1H-pyrazol-4-yl)thiazole-4-carboxamide;

VI-5: 1-methylcyclopropyl 4-(4-((1-methyl-3-(pyridin-2-yl)-1H-pyrazol-4-yl)carbamoyl)thiazol-2-yl)-1H-pyrazole-1-carboxylate;

VI-6: 1-((4-methoxybenzypoxy)-2-methylpropan-2-yl4-(4-((1-methyl-3-(pyridin-2-yl)-1H-pyrazol-4-yl)carbamoyl)thiazol-2-yl)-1H-pyrazole-1-carboxylate;

VI-7: diethyl ((4-(4-((1-methyl-3-(pyridin-2-yl)-1H-pyrazol-4-yl)carbamoyl)thiazol-2-yl)-1H-pyrazol-1-yl)methyl)phosphonate;

VI-8: sodium ((4-(4-((1-methyl-3-(pyridin-2-yl)-1H-pyrazol-4-yl)carbamoyl)thiazol-2-yl)-1H-pyrazol-1-yl)methyl)phosphonate;

VI-9: ((4-(4-((1-methyl-3-(pyridin-2-yl)-1H-pyrazol-4-yl)carbamoyl)thiazol-2-yl)-1H-pyrazol-1-yl)methyl)phosphonic acid;

VI-10: 2-(1H-pyrazol-4-yl)-N-(3-(pyridin-2-yl)-1-(tetrahydro-2H-pyran-4-yl)-1H-pyrazol-4-yl)thiazole-4-carboxamide;

VI-11: N-(1-((1,3-cis)-3-ethoxycyclobutyl)-3-(pyridin-2-yl)-1H-pyrazol-4-yl)-2-(1H-pyrazol-4-yl)thiazole-4-carboxamide;

VI-12: N-(1-((1,3-trans)-3-ethoxycyclobutyl)-3-(pyridin-2-yl)-1H-pyrazol-4-yl)-2-(1H-pyrazol-4-yl)thiazole-4-carboxamide;

VI-13: N-(1-((1,3-cis)-3-ethoxycyclobutyl)-3-(pyridin-2-yl)-1H-pyrazol-4-yl)-2-(1-methyl-1H-pyrazol-4-yl)thiazole-4-carboxamide;

VI-14: N-(1-((1,3-cis)-3-hydroxycyclobutyl)-3-(pyridin-2-yl)-1H-pyrazol-4-yl)-2-(1H-pyrazol-4-yl)thiazole-4-carboxamide;

VI-15: N-(1-((1s,3s)-3-(dimethylamino)cyclobutyl)-3-(pyridin-2-yl)-1H-pyrazol-4-yl)-2-(1H-pyrazol-4-yl)thiazole-4-carboxamide;

VI-16: (4-(4-((1-((1,3-cis)-3-ethoxycyclobutyl)-3-(pyridin-2-yl)-1H-pyrazol-4-yl)carbamoyl)thiazol-2-yl)-1H-pyrazol-1-yl)methyl phosphate bis-sodium salt;

VI-17: (4-(4-((1-((1s,3s)-3-ethoxycyclobutyl)-3-(pyridin-2-yl)-1H-pyrazol-4-yl)carbamoyl)thiazol-2-yl)-1H-pyrazol-1-yl)methyl dihydrogen phosphate;

VI-18: N-(1-(2-ethoxyethyl)-3-(pyridin-2-yl)-1H-pyrazol-4-yl)-2-(1H-pyrazol-4-yl)thiazole-4-carboxamide, formic acid salt;

VI-19: N-(1-(2-ethoxyethyl)-3-(pyridin-2-yl)-1H-pyrazol-4-yl)-2-(5-(trifluoromethyl)-1H-pyrazol-4-yl)thiazole-4-carboxamide, formic acid salt;

VI-20: N-(1-(2-ethoxyethyl)-3-(pyridin-2-yl)-1H-pyrazol-4-yl)-2-(5-(trifluoromethyl)-1H-pyrazol-4-yl)thiazole-4-carboxamide;

VI-21: N-(1-(2-ethoxyethyl)-3-(pyridin-2-yl)-1H-pyrazol-4-yl)-2-(3-methyl-1H-pyrazol-4-yl)thiazole-4-carboxamide, formic acid salt;

VI-22: N-(1-(2-ethoxyethyl)-3-(pyridin-2-yl)-1H-pyrazol-4-yl)-2-(3-methyl-1H-pyrazol-4-yl)thiazole-4-carboxamide;

VI-23: 2-(3,5-dimethyl-1H-pyrazol-4-yl)-N-(1-(2-ethoxyethyl)-3-(pyridin-2-yl)-1H-pyrazol-4-yl)thiazole-4-carboxamide, formic acid salt;

VI-24: 2-(3,5-dimethyl-1H-pyrazol-4-yl)-N-(1-(2-ethoxyethyl)-3-(pyridin-2-yl)-1H-pyrazol-4-yl)thiazole-4-carboxamide;

VI-25: N-(1-(2-ethoxyethyl)-3-(pyridin-2-yl)-1H-pyrazol-4-yl)-2-(1H-pyrazol-4-yl)thiazole-4-carboxamide;

VI-26: N-(1-methyl-3-(pyridin-2-yl)-1H-pyrazol-4-yl)-2-(1H-pyrazol-4-yl)thiazole-4-carboxamide;

VI-27: 2-(3-methyl-1H-pyrazol-4-yl)-N-(1-methyl-3-(pyridin-2-yl)-1H-pyrazol-4-yl)thiazole-4-carboxamide;

VI-28: N-(1-(2-methoxyethyl)-3-(pyridin-2-yl)-1H-pyrazol-4-yl)-2-(1H-pyrazol-4-yl)thiazole-4-carboxamide;

VI-29: N-(1-(2-methoxyethyl)-3-(pyridin-2-yl)-1H-pyrazol-4-yl)-2-(3-methyl-1H-pyrazol-4-yl)thiazole-4-carboxamide, formic acid salt;

VI-30: N-(1-(2-methoxyethyl)-3-(pyridin-2-yl)-1H-pyrazol-4-yl)-2-(3-methyl-1H-pyrazol-4-yl)thiazole-4-carboxamide;

VI-31: N-(1-(2-ethoxyethyl)-3-(pyridin-2-yl)-1H-pyrazol-4-yl)-2-(3-methyl-1H-pyrazol-4-yl)thiazole-4-carboxamide;

VI-32: 2-(1H-pyrazol-4-yl)-N-(3-(pyridin-2-yl)-1H-pyrazol-4-yl)thiazole-4-carboxamide formate;

VI-33: 2-(1H-pyrazol-4-yl)-N-(3-(pyridin-2-yl)-1H-pyrazol-4-yl)thiazole-4-carboxamide;

VI-34: N-(1-(oxetan-3-yl)-3-(pyridin-2-yl)-1H-pyrazol-4-yl)-2-(1H-pyrazol-4-yl)thiazole-4-carboxamide;

VI-35: (4-(4-((1-methyl-3-(pyridin-2-yl)-1H-pyrazol-4-yl)carbamoyl)thiazol-2-yl)-1H-pyrazol-1-yl)methyl dihydrogen phosphate;

VI-36: Sodium (4-(4-((1-methyl-3-(pyridin-2-yl)-1H-pyrazol-4-yl)carbamoyl)thiazol-2-yl)-1H-pyrazol-1-yl)methyl phosphate;

VI-37: N-(1-(2-(2-methoxyethoxy)ethyl)-3-(pyridin-2-yl)-1H-pyrazol-4-yl)-2-(1H-pyrazol-4-yl)thiazole-4-carboxamide;

VI-38: potassium (4-(4-((1-methyl-3-(pyridin-2-yl)-1H-pyrazol-4-yl)carbamoyl)thiazol-2-yl)-1H-pyrazol-1-yl)methyl phosphate;

VI-39: N-(1-(2-(2-methoxyethoxy)ethyl)-3-(pyridin-2-yl)-1H-pyrazol-4-yl)-2-(3-methyl-1H-pyrazol-4-yl)thiazole-4-carboxamide formate;

VI-40: N-(1-(2-(2-methoxyethoxy)ethyl)-3-(pyridin-2-yl)-1H-pyrazol-4-yl)-2-(3-methyl-1H-pyrazol-4-yl)thiazole-4-carboxamide;

VI-41: 2-(3-methyl-1H-pyrazol-4-yl)-N-(1-(oxetan-3-yl)-3-(pyridin-2-yl)-1H-pyrazol-4-yl)thiazole-4-carboxamide, formic acid salt;

VI-42: 2-(3-methyl-1H-pyrazol-4-yl)-N-(1-(oxetan-3-yl)-3-(pyridin-2-yl)-1H-pyrazol-4-yl)thiazole-4-carboxamide;

VI-43: 2-(1H-pyrazol-4-yl)-N-(3-(pyridin-2-yl)-1-(tetrahydrofuran-3-yl)-1H-pyrazol-4-yl)thiazole-4-carboxamide formate;

VI-44: 2-(1H-pyrazol-4-yl)-N-(3-(pyridin-2-yl)-1-(tetrahydrofuran-3-yl)-1H-pyrazol-4-yl)thiazole-4-carboxamide;

VI-45: 2-(3-methyl-1H-pyrazol-4-yl)-N-(3-(pyridin-2-yl)-1-((tetrahydro-2H-pyran-4-yl)methyl)-1H-pyrazol-4-yl)thiazole-4-carboxamide formate;

VI-46: 2-(3-methyl-1H-pyrazol-4-yl)-N-(3-(pyridin-2-yl)-1-((tetrahydro-2H-pyran-4-yl)methyl)-1H-pyrazol-4-yl)thiazole-4-carboxamide;

VI-47: N-(1-((3-(hydroxymethypoxetan-3-yl)methyl)-3-(pyridin-2-yl)-1H-pyrazol-4-yl)-2-(3-methyl-1H-pyrazol-4-yl)thiazole-4-carboxamide formate;

VI-48: N-(1-((3-(hydroxymethypoxetan-3-yl)methyl)-3-(pyridin-2-yl)-1H-pyrazol-4-yl)-2-(3-methyl-1H-pyrazol-4-yl)thiazole-4-carboxamide;

VI-49: N-(1-(2-(diethylamino)ethyl)-3-(pyridin-2-yl)-1H-pyrazol-4-yl)-2-(1H-pyrazol-4-yl)thiazole-4-carboxamide, formic acid salt;

VI-50: N-(1-(2-(diethylamino)ethyl)-3-(pyridin-2-yl)-1H-pyrazol-4-yl)-2-(1H-pyrazol-4-yl)thiazole-4-carboxamide;

VI-51: 2-(1-(4-methoxybenzyl)-1H-pyrazol-4-yl)-N-(1-(3-methoxycyclobutyl)-3-(pyridin-2-yl)-1H-pyrazol-4-ypthiazole-4-carboxamide;

VI-52: N-(1-(2-fluoroethyl)-3-(pyridin-2-yl)-1H-pyrazol-4-yl)-2-(1-(4-methoxybenzyl)-1H-pyrazol-4-ypthiazole-4-carboxamide;

VI-53: 2-(1-(4-methoxybenzyl)-1H-pyrazol-4-yl)-N-(3-(pyridin-2-yl)-1H-pyrazol-4-yl)thiazole-4-carboxamide;

VI-54: tert-Butyl-3-[4-{2-(1H-pyrazole-4-yl)thiazole-2-carboxamido}-3-(pyridine-2-yl)-1H-pyrazol-1-yl]azetidine-1-carboxylate, free base;

VI-55: N-{1-(Azetidin-3-yl)-3-(pyridine-2-yl)-1H-pyrazol-4-yl}-2-(1H-pyrazol-4-yl)thiazole-4-carboxamide, TFA salt;

VI-56: N-{1-(Azetidin-3-yl)-3-(pyridine-2-yl)-1H-pyrazol-4-yl}-2-(1H-pyrazol-4-yl)thiazole-4-carboxamide;

VI-57: N-{1-(3-Methoxycyclobutyl)-3-(pyridine-2-yl)-1H-pyrazol-4-yl}-2-(1H-pyrazol-4-yl)thiazole-4-carboxamide, free base, Cis isomer;

VI-58: N-(3-(5-methoxypyridin-2-yl)-1-methyl -1H-pyrazol-4-yl)-2-(1H-pyrazol-4-yl)thiazole-4-carboxamide;

VI-59: N-(1-isopropyl-3-(pyridin-2-yl)-1H-pyrazol-4-yl)-2-(1H-pyrazol-4-yl)thiazole-4-carboxamide;

VI-60: N-(1-(2-morpholinoethyl)-3-(pyridin-2-yl)-1H-pyrazol-4-yl)-2-(1H-pyrazol-4-yl)thiazole-4-carboxamide;

VI-61: N-(1-(2-(4-methylpiperazin-1-yl)ethyl)-3-(pyridin-2-yl)-1H-pyrazol-4-yl)-2-(1H-pyrazol-4-yl)thiazole-4-carboxamide;

VI-65: N-(3-(3-fluoropyridin-2-yl)-1-((1s,3 s)-3-hydroxycyclobutyl)-1H-pyrazol-4-yl)-2-(1H-pyrazol-4-yl)thiazole-4-carboxamide;

VI-66: 2-(1H-pyrazol-3-yl)-N-(3-(pyridin-2-yl)-1-(2-(2,2,2-trifluoroethoxy)ethyl)-1H-pyrazol-4-yl)thiazole-4-carboxamide;

VI-71: N-(1-((1s,3s)-3-ethoxycyclobutyl)-3-(pyridin-2-yl)-1H-pyrazol-4-yl)-2-(5-fluoro-1-((2-(trimethylsilyl)ethoxy)methyl)-1H-pyrazol-4-yl)thiazole-4-carboxamide;

VI-72: N-(1-((1s,3s)-3-ethoxycyclobutyl)-3-(pyridin-2-yl)-1H-pyrazol-4-yl)-2-(5-fluoro-1H-pyrazol-4-yl)thiazole-4-carboxamide;

VI-73: N-(1-((1s,3s)-3-ethoxycyclobutyl)-3-(pyridin-2-yl)-1H-pyrazol-4-yl)-2-(5-fluoro-1H-pyrazol-4-yl)thiazole-4-carboxamide formate;

VI-76: N-(1-((1s,3s)-3-isopropoxycyclobutyl)-3-(pyridin-2-yl)-1H-pyrazol-4-yl)-2-(1H-pyrazol-4-yl)thiazole-4-carboxamide;

VI-77: potassium (4-(4-((1-((1s,3 s)-3-ethoxycyclobutyl)-3-(pyridin-2-yl)-1H-pyrazol-4-yl)carbamoyl)thiazol-2-yl)-1H-pyrazol-1-yl)methyl phosphate;

VI-78: calcium (4-(4-((1-((1s,3s)-3-ethoxycyclobutyl)-3-(pyridin-2-yl)-1H-pyrazol-4-yl)carbamoyl)thiazol-2-yl)-1H-pyrazol-1-yl)methyl phosphate;

VI-79: N-(1-((1r,2r)-3-hydroxy-3-methylcyclobutyl)-3-(pyridin-2-yl)-1H-pyrazol-4-yl)-2-(1H-pyrazol-4-yl)thiazole-4-carboxamide;

VI-80: ammonium (4-(4-((1-((1s,3s)-3-ethoxycyclobutyl)-3-(pyridin-2-yl)-1H-pyrazol-4-yl)carbamoyl)thiazol-2-yl)-1H-pyrazol-1-yl)methyl phosphate;

VI-81: 5-amino-5-carboxypentan-1-aminium (4-(4-((1-((1s,3s)-3-ethoxycyclobutyl)-3-(pyridin-2-yl)-1H-pyrazol-4-yl)carbamoyl)thiazol-2-yl)-1H-pyrazol-1-yl)methyl phosphate;

VI-82: 1-(4-amino-4-carboxybutyl)guanidinium (4-(4-((1-((1s,3s)-3-ethoxycyclobutyl)-3-(pyridin-2-yl)-1H-pyrazol-4-yl)carbamoyl)thiazol-2-yl)-1H-pyrazol-1-yl)methyl phosphate;

VI-83: (4-(4-((1-((1s,3s)-3-ethoxycyclobutyl)-3-(pyridin-2-yl)-1H-pyrazol-4-yl)carbamoyl)thiazol-2-yl)-1H-pyrazol-1-yl)methyl dihydrogen phosphate;

VI-84: 1,3-dihydroxy-2-(hydroxymethyl)propan-2-aminium (4-(4-((1-((1 s,3 s)-3-ethoxycyclobutyl)-3-(pyridin-2-yl)-1H-pyrazol-4-yl)carbamoyl)thiazol-2-yl)-1H-pyrazol-1-yl)methyl hydrogen phosphate;

VI-85: triethylammonium (4-(4-((1-((1 s,3 s)-3-ethoxycyclobutyl)-3-(pyridin-2-yl)-1H-pyrazol-4-yl)carbamoyl)thiazol-2-yl)-1H-pyrazol-1-yl)methyl hydrogen phosphate;

VI-86: N-(1-((1 s,3 s)-3-ethoxycyclobutyl)-3-(5-(trifluoromethyppyridin-2-yl)-1H-pyrazol-4-yl)-2-(1H-pyrazol-4-yl)thiazole -4-carboxamide;

VI-87: N-(1-(3-hydroxy-3-methylcyclobutyl)-3-(pyridin-2-yl)-1H-pyrazol-4-yl)-2-(1H-pyrazol-4-yl)thiazole-4-carboxamide;

VI-88: N-(1-(difluoromethyl)-3-(pyridin-2-yl)-1H-pyrazol-4-yl)-2-(1H-pyrazol-4-yl)thiazole -4-carboxamide;

VI-89: N-(1-((1 s,3 s)-3-ethoxycyclobutyl)-3-(3-(trifluoromethyppyridin-2-yl)-1H-pyrazol-4-yl)-2-(1H-pyrazol-4-yl)thiazole -4-carboxamide;

VI-90: N-(1-((1 s,3 s)-3-ethoxycyclobutyl)-3-(6-(trifluoromethyppyridin-2-yl)-1H-pyrazol-4-yl)-2-(1H-pyrazol-4-yl)thiazole-4-carboxamide;

VI-91: N-(1-((1 s,3 s)-3-ethoxycyclobutyl)-3-(pyridin-2-yl)-1H-pyrazol-4-yl)-2-(3-(trifluoromethyl)-1H-pyrazol-4-yl)thiazole-4-carboxamide;

VI-92: N-(1-((1 s,3 s)-3-ethoxycyclobutyl)-3-(pyridin-2-yl)-1H-pyrazol-4-yl)-2-(3-methyl-1H-pyrazol-4-yl)thiazole -4-carboxamide;

VI-93: 2-(3,5-dimethyl-1H-pyrazol-4-yl)-N-(1-((1s,3s)-3-ethoxycyclobutyl)-3-(pyridin-2-yl)-1H-pyrazol-4-yl)thiazole-4-carboxamide;

VI-94: 2-(1H-pyrazol-4-yl)-N-(3-(pyridin-2-yl)-1-(2,2,2-trifluoroethyl)-1H-pyrazol-4-yl)thiazole -4-carboxamide;

VI-95: N-(1-(difluoromethyl)-3-(pyridin-2-yl)-1H-pyrazol-4-yl)-2-(3-(trifluoromethyl)-1H-pyrazol-4-yl)thiazole-4-carboxamide;

VI-96: N-(1-(difluoromethyl)-3-(pyridin-2-yl)-1H-pyrazol-4-yl)-2-(3-methyl-1H-pyrazol-4-yl)thiazole-4-carboxamide;

VI-97: N-(1-(difluoromethyl)-3-(pyridin-2-yl)-1H-pyrazol-4-yl)-2-(1-(2,2,2-trifluoroethyl)-1H-pyrazol-4-yl)thiazole -4-carboxamide;

VI-98: 2-(1-(difluoromethyl)-1H-pyrazol-4-yl)-N-(1-(difluoromethyl)-3-(pyridin-2-yl)-1H-pyrazol-4-yl)thiazole-4-carboxamide;

VI-99: N-(1-((1s,3s)-3-ethoxycyclobutyl)-3-(6-(trifluoromethyppyridin-2-yl)-1H-pyrazol-4-yl)-2-(3-methyl-1H-pyrazol-4-yl)thiazole-4-carboxamide;

VI-100: 2-(3-methyl-1H-pyrazol-4-yl)-N-(3-(pyridin-2-yl)-1-(2,2,2-trifluoroethyl)-1H-pyrazol-4-yl)thiazole-4-carboxamide;

VI-103: 2-(1-(4-methoxybenzyl)-1H-pyrazol-4-yl)-N-(3-(pyridin-2-yl)-1-(3,3,3-trifluoro-2-hydroxypropyl)-1H-pyrazol-4-ypthiazole-4-carboxamide formate;

VI-104: 2-(1-(4-methoxybenzyl)-1H-pyrazol-4-yl)-N-(3-(pyridin-2-yl)-1-(3,3,3-trifluoro-2-hydroxypropyl)-1H-pyrazol-4-ypthiazole-4-carboxamide;

VI-105: N-(1-(dimethylcarbamoyl)-3-(pyridin-2-yl)-1H-pyrazol-4-yl)-2-(1-(4-methoxybenzyl)-1H-pyrazol-4-yl)thiazole-4-carboxamide formate;

VI-106: N-(1-(dimethylcarbamoyl)-3-(pyridin-2-yl)-1H-pyrazol-4-yl)-2-(1-(4-methoxybenzyl)-1H-pyrazol-4-yl)thiazole-4-carboxamide;

VI-107: 2-(1-(4-methoxybenzyl)-1H-pyrazol-4-yl)-N-(3-(pyridin-2-yl)-1-(3,3,3-trifluoro-2-hydroxy-2-(trifluoromethyl)propyl)-1H-pyrazol-4-ypthiazole-4-carboxamide formate;

VI-108: 2-(1-(4-methoxybenzyl)-1H-pyrazol-4-yl)-N-(3-(pyridin-2-yl)-1-(3,3,3-trifluoro-2-hydroxy-2-(trifluoromethyl)propyl)-1H-pyrazol-4-ypthiazole-4-carboxamide;

VI-117: N-(1-(2-(diethylamino)ethyl)-3-(pyridin-2-yl)-1H-pyrazol-4-yl)-2-(1H-pyrazol-4-yl)thiazole-4-carboxamide;

VI-118: N-(1-(2-(2-fluoroethoxy)ethyl)-3-(pyridin-2-yl)-1H-pyrazol-4-yl)-2-(1H-pyrazol-4-yl)thiazole-4-carboxamide formate;

VI-119: N-(1-(2-(2-fluoroethoxy)ethyl)-3-(pyridin-2-yl)-1H-pyrazol-4-yl)-2-(1H-pyrazol-4-yl)thiazole-4-carboxamide;

VI-120: N-(1-benzyl-3-(pyridin-2-yl)-1H-pyrazol-4-yl)-2-(1H-pyrazol-4-yl)thiazole-4-carboxamide;

VI-121: N-(1-cyclobutyl-3-(pyridin-2-yl)-1H-pyrazol-4-yl)-2-(1H-pyrazol-4-yl)thiazole-4-carboxamide;

VI-122: N-(1-(2-(2,2-difluoroethoxy)ethyl)-3-(pyridin-2-yl)-1H-pyrazol-4-yl)-2-(1H-pyrazol-4-yl)thiazole-4-carboxamide;

VI-123: N-(1-(((1r,2r)-3-hydroxycyclobutyl)methyl)-3-(pyridin-2-yl)-1H-pyrazol-4-yl)-2-(1H-pyrazol-4-yl)thiazole-4-carboxamide formate;

VI-124: N-(1-(((1r,2r)-3-hydroxycyclobutyl)methyl)-3-(pyridin-2-yl)-1H-pyrazol-4-yl)-2-(1H-pyrazol-4-yl)thiazole-4-carboxamide;

VI-125: N-(1-(dimethylcarbamoyl)-3-(pyridin-2-yl)-1H-pyrazol-4-yl)-2-(1H-pyrazol-4-yl)thiazole-4-carboxamide formate;

VI-126: N-(1-(dimethylcarbamoyl)-3-(pyridin-2-yl)-1H-pyrazol-4-yl)-2-(1H-pyrazol-4-yl)thiazole-4-carboxamide;

VI-127: N-(1-((1s,3s)-3-(ethoxy-d5)cyclobutyl)-3-(pyridin-2-yl)-1H-pyrazol-4-yl)-2-(1H-pyrazol-4-yl)thiazole-4-carboxamide;

VI-128: N-(1-(diethylcarbamoyl)-3-(pyridin-2-yl)-1H-pyrazol-4-yl)-2-(1H-pyrazol-4-yl)thiazole-4-carboxamide;

VI-129: N-(1-(morpholine-4-carbonyl)-3-(pyridin-2-yl)-1H-pyrazol-4-yl)-2-(1H-pyrazol-4-yl)thiazole-4-carboxamide;

VI-130: N-(1-((1s,3s)-3-(2-fluoroethoxy)cyclobutyl)-3-(pyridin-2-yl)-1H-pyrazol-4-yl)-2-(1H-pyrazol-4-yl)thiazole-4-carboxamide;

VI-131: N-(1-(morpholine-4-carbonyl)-3-(pyridin-2-yl)-1H-pyrazol-4-yl)-2-(1H-pyrazol-4-yl)thiazole-4-carboxamide formate;

VI-132: N-(1-(3-fluorocyclobut-2-en-1-yl)-3-(pyridin-2-yl)-1H-pyrazol-4-yl)-2-(1H-pyrazol-4-yl)thiazole-4-carboxamide;

VI-133: N-(1-(3-fluorocyclobut-2-en-1-yl)-3-(pyridin-2-yl)-1H-pyrazol-4-yl)-2-(1H-pyrazol-4-yl)thiazole-4-carboxamide formate;

VI-134: N-(1-(3,3-difluorocyclobutyl)-3-(pyridin-2-yl)-1H-pyrazol-4-yl)-2-(1H-pyrazol-4-yl)thiazole-4-carboxamide formate;

VI-135: N-(1-(3,3-difluorocyclobutyl)-3-(pyridin-2-yl)-1H-pyrazol-4-yl)-2-(1H-pyrazol-4-yl)thiazole-4-carboxamide;

VI-140: N-(3-(3-fluoropyridin-2-yl)-1-(1,4-dioxaspiro[4.5]decan-8-yl)-1H-pyrazol-4-yl)-2-(1H-pyrazol-4-yl)thiazole-4-carboxamide;

VI-141: 2-(1H-pyrazol-4-yl)-N-(3-(pyridin-2-yl)-1-((1r,2r)-3-(2,2,2-trifluoroethoxy)cyclobutyl)-1H-pyrazol-4-yl)thiazole-4-carboxamide formate;

VI-142: 2-(1H-pyrazol-4-yl)-N-(3-(pyridin-2-yl)-1-((1r,2r)-3-(2,2,2-trifluoroethoxy)cyclobutyl)-1H-pyrazol-4-yl)thiazole-4-carboxamide;

VI-143: N-(1-((1r,4r)-4-hydroxycyclohexyl)-3-(pyridin-2-yl)-1H-pyrazol-4-yl)-2-(1H-pyrazol-4-yl)thiazole-4-carboxamide formate;

VI-144: N-(1-((1r,4r)-4-hydroxycyclohexyl)-3-(pyridin-2-yl)-1H-pyrazol-4-yl)-2-(1H-pyrazol-4-yl)thiazole-4-carboxamide;

VI-145: N-(1-((1r,4r)-4-ethoxycyclohexyl)-3-(pyridin-2-yl)-1H-pyrazol-4-yl)-2-(1H-pyrazol-4-yl)thiazole-4-carboxamide formate;

VI-146: N-(1-((1r,4r)-4-ethoxycyclohexyl)-3-(pyridin-2-yl)-1H-pyrazol-4-yl)-2-(1H-pyrazol-4-yl)thiazole-4-carboxamide;

VI-147: N-(1-((1S,3R)-3-ethoxycyclopentyl)-3-(pyridin-2-yl)-1H-pyrazol-4-yl)-2-(1H-pyrazol-4-yl)thiazole-4-carboxamide formate;

VI-148: N-(1-((1S,3R)-3-ethoxycyclopentyl)-3-(pyridin-2-yl)-1H-pyrazol-4-yl)-2-(1H-pyrazol-4-yl)thiazole-4-carboxamide;

VI-149: N-(1-((1S,3R)-3-hydroxycyclopentyl)-3-(pyridin-2-yl)-1H-pyrazol-4-yl)-2-(1H-pyrazol-4-yl)thiazole-4-carboxamide formate;

VI-150: N-(1-((1S,3R)-3-hydroxycyclopentyl)-3-(pyridin-2-yl)-1H-pyrazol-4-yl)-2-(1H-pyrazol-4-yl)thiazole-4-carboxamide;

VI-151: N-(1-((1S,3S)-3-hydroxycyclopentyl)-3-(pyridin-2-yl)-1H-pyrazol-4-yl)-2-(1H-pyrazol-4-yl)thiazole-4-carboxamide formate;

VI-152: N-(1-((1S,3S)-3-hydroxycyclopentyl)-3-(pyridin-2-yl)-1H-pyrazol-4-yl)-2-(1H-pyrazol-4-yl)thiazole-4-carboxamide;

VI-153: N-(1-((1s,3s)-3-ethoxycyclobutyl)-3-(5-fluoropyridin-2-yl)-1H-pyrazol-4-yl)-2-(1H-pyrazol-4-yl)thiazole-4-carboxamide formate;

VI-154: N-(1-((1s,3s)-3-ethoxycyclobutyl)-3-(5-fluoropyridin-2-yl)-1H-pyrazol-4-yl)-2-(1H-pyrazol-4-yl)thiazole-4-carboxamide;

VI-155: N-(1-((1S,3R)-3-ethoxy-2-fluorocyclobutyl)-3-(pyridin-2-yl)-1H-pyrazol-4-yl)-2-(1H-pyrazol-4-yl)thiazole-4-carboxamide formate;

VI-156: N-(1-((1S,3R)-3-ethoxy-2-fluorocyclobutyl)-3-(pyridin-2-yl)-1H-pyrazol-4-yl)-2-(1H-pyrazol-4-yl)thiazole-4-carboxamide;

VI-157: N-(1-((1s,3s)-3-ethoxycyclobutyl)-3-(3-fluoropyridin-2-yl)-1H-pyrazol-4-yl)-2-(1H-pyrazol-4-yl)thiazole-4-carboxamide;

VI-158: N-(1-((1s,3s)-3-ethoxycyclobutyl)-3-(4-fluoropyridin-2-yl)-1H-pyrazol-4-yl)-2-(1H-pyrazol-4-yl)thiazole-4-carboxamide formate;

VI-159: N-(1-((1s,3s)-3-ethoxycyclobutyl)-3-(4-fluoropyridin-2-yl)-1H-pyrazol-4-yl)-2-(1H-pyrazol-4-yl)thiazole-4-carboxamide;

VI-160: N-(1-((1s,3s)-3-ethoxycyclobutyl)-3-(6-fluoropyridin-2-yl)-1H-pyrazol-4-yl)-2-(1H-pyrazol-4-yl)thiazole-4-carboxamide;

VI-161: 2-(1H-pyrazol-4-yl)-N-(3-(pyridin-2-yl)-1-((1s,3s)-3-(2,2,2-trifluoroethoxy)cyclobutyl)-1H-pyrazol-4-yl)thiazole-4-carboxamide formate;

VI-162: 2-(1H-pyrazol-4-yl)-N-(3-(pyridin-2-yl)-1-((1s,3s)-3-(2,2,2-trifluoroethoxy)cyclobutyl)-1H-pyrazol-4-yl)thiazole-4-carboxamide;

VI-163: (4-(4-((1-((1s,3s)-3-ethoxycyclobutyl)-3-(3-fluoropyridin-2-yl)-1H-pyrazol-4-yl)carbamoyl)thiazol-2-yl)-1H-pyrazol-1-yl)methyl dihydrogen phosphate;\

VI-164: sodium (4-(4-((1-((1s,3s)-3-ethoxycyclobutyl)-3-(3-fluoropyridin-2-yl)-1H-pyrazol-4-yl)carbamoyl)thiazol-2-yl)-1H-pyrazol-1-yl)methyl phosphate;

VI-165: N-(3-(3-fluoropyridin-2-yl)-1-((1s,3 s)-3-(2,2,2-trifluoroethoxy)cyclobutyl)-1H-pyrazol-4-yl)-2-(1H-pyrazol-4-yl)thiazole-4-carboxamide formate;

VI-166: N-(3-(3-fluoropyridin-2-yl)-1-((1s,3 s)-3-(2,2,2-trifluoroethoxy)cyclobutyl)-1H-pyrazol-4-yl)-2-(1H-pyrazol-4-yl)thiazole-4-carboxamide;

VI-167: N-(3-(3-fluoropyridin-2-yl)-1-((1r,2r)-3-(2,2,2-trifluoroethoxy)cyclobutyl)-1H-pyrazol-4-yl)-2-(1H-pyrazol-4-yl)thiazole-4-carboxamide formate;

VI-168: N-(3-(3-fluoropyridin-2-yl)-1-((1r,2r)-3-(2,2,2-trifluoroethoxy)cyclobutyl)-1H-pyrazol-4-yl)-2-(1H-pyrazol-4-yl)thiazole-4-carboxamide;

VI-169: N-(1-((1r,4r)-4-ethoxycyclohexyl)-3-(3-fluoropyridin-2-yl)-1H-pyrazol-4-yl)-2-(1H-pyrazol-4-yl)thiazole-4-carboxamide;

VI-170: N-(3-(6-fluoropyridin-2-yl)-1-((1s,3s)-3-(2,2,2-trifluoroethoxy)cyclobutyl)-1H-pyrazol-4-yl)-2-(1H-pyrazol-4-yl)thiazole-4-carboxamide formate;

VI-171: N-(3-(6-fluoropyridin-2-yl)-1-((1s,3s)-3-(2,2,2-trifluoroethoxy)cyclobutyl)-1H-pyrazol-4-yl)-2-(1H-pyrazol-4-yl)thiazole-4-carboxamide;

VI-172: N-(3-(6-fluoropyridin-2-yl)-1-((1s,3s)-3-hydroxycyclobutyl)-1H-pyrazol-4-yl)-2-(1H-pyrazol-4-yl)thiazole-4-carboxamide;

VI-173: (4-(4-((1-((1s,3s)-3-ethoxycyclobutyl)-3-(6-fluoropyridin-2-yl)-1H-pyrazol-4-yl)carbamoyl)thiazol-2-yl)-1H-pyrazol-1-yl)methyl dihydrogen phosphate;

VI-174: N-(3-(3,6-difluoropyridin-2-yl)-1-((1s,3s)-3-ethoxycyclobutyl)-1H-pyrazol-4-yl)-2-(1H-pyrazol-4-yl)thiazole-4-carboxamide;

VI-175: N-(1-((1s,4s)-4-ethoxycyclohexyl)-3-(3-fluoropyridin-2-yl)-1H-pyrazol-4-yl)-2-(1H-pyrazol-4-yl)thiazole-4-carboxamide;

VI-176: N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide;

VI-177:N-(3-(3,6-difluoropyridin-2-yl)-1-((1s,4s)-4-ethoxycyclohexyl)-1H-pyrazol-4-yl)-2-(1H-pyrazol-4-yl)thiazole-4-carboxamide; or

VI-180: N-(3-(3,5-difluoropyridin-2-yl)-1-((1r,4r)-4-ethoxycyclohexyl)-1H-pyrazol-4-yl)-2-(1H-pyrazol-4-yl)thiazole-4-carboxamide.

In particular embodiments, the compound is

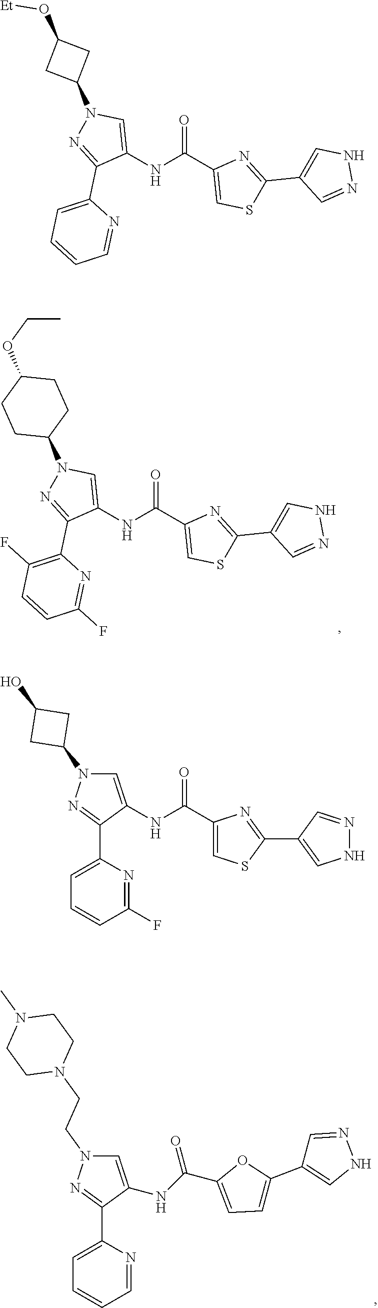 [SMILES: CCO[C@H]1C[C@@H](n2cc(NC(=O)c3csc(-c4cn[nH]c4)n3)c(-c3ccccn3)n2)C1 ; CCO[C@H]1CC[C@H](n2cc(NC(=O)c3csc(-c4cn[nH]c4)n3)c(-c3nc(F)ccc3F)n2)CC1 ; O=C(Nc1cn([C@H]2C[C@@H](O)C2)nc1-c1cccc(F)n1)c1csc(-c2cn[nH]c2)n1 ; CN1CCN(CCn2cc(NC(=O)c3ccc(-c4cn[nH]c4)o3)c(-c3ccccn3)n2)CC1]

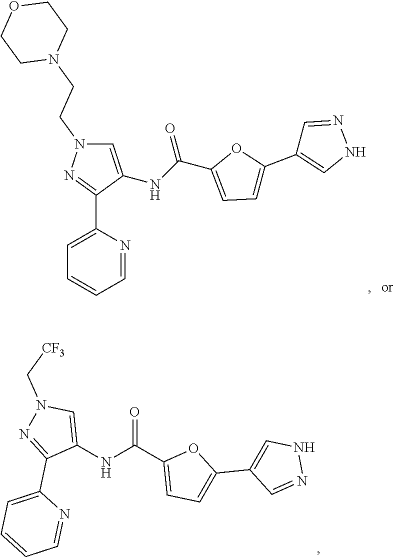 [SMILES: O=C(Nc1cn(CCN2CCOCC2)nc1-c1ccccn1)c1ccc(-c2cn[nH]c2)o1 ; O=C(Nc1cn(CC(F)(F)F)nc1-c1ccccn1)c1ccc(-c2cn[nH]c2)o1]

or a pharmaceutically acceptable salt thereof.

Additional information concerning pyrazole compounds, such as compounds according to Formula IV, can be found in U.S. Pat. No. 9,982,000, which is incorporated herein by reference in its entirety.

### D. Additional Pyrazole Compounds

Disclosed herein are pyrazole compounds, methods of making the compounds, and methods of using the compounds. In one embodiment, the disclosed compounds are tyrosine kinase inhibitors and/or may be useful in blocking one or more cytokine signaling pathways, such as the IL-17 signaling pathway. For certain embodiments, the pyrazole compounds are useful for treating conditions in which inhibition of an interleukin-1 receptor-associated kinase (IRAK) pathway is therapeutically useful. In some embodiments, the compounds inhibit an IRAK protein, such as IRAK1, IRAK2, IRAK3 or IRAK4. In other embodiments, the compounds are useful for delivering an IRAK inhibitor compound, and/or may be a prodrug of an IRAK inhibitor. In certain embodiments, the pyrazole compound is a prodrug of

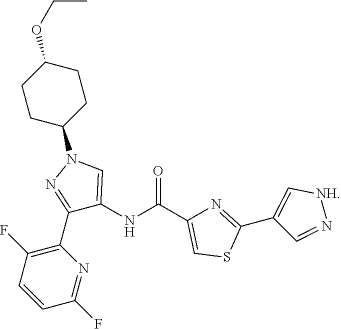 [SMILES: CCO[C@H]1CC[C@H](n2cc(NC(=O)c3csc(-c4cn[nH]c4)n3)c(-c3nc(F)ccc3F)n2)CC1]

In some embodiments, the pyrazole compound has a general Formula VII

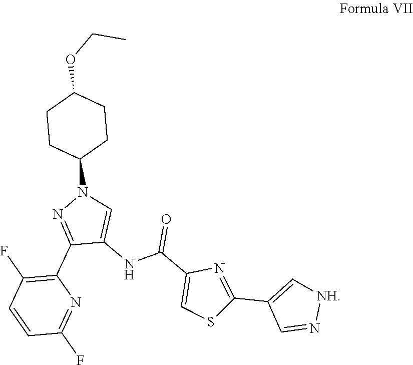

or a salt, solvate or N-oxide thereof. With respect to Formula VII, R is H, aliphatic, acyl, heterocyclyl, carboxyl ester, amide, alkyl phosphoramidate, or alkyl phosphate. In some embodiments, R is not H, or alternatively, R is H and the compound is a salt. In other embodiments, R is alkyl, acyl, carboxyl ester, amide, nonaromatic heterocyclyl, alkyl phosphoramidate, or alkyl phosphate. A person of ordinary skill in the art understands that compounds where R is not H may act a prodrug of the compound where R is H, for example, when administered to a subject.

In some embodiments, R is H, C₁₋₄alkyl phosphate, C₁₋₄alkyl phosphoramidate, C₁₋₆alkyl, C₁₋₆acyl, —C(O)O—C₁₋₆aliphatic, —C(O)N(R^{b})₂, or 5- or 6-membered nonaromatic heterocyclyl, but in certain embodiments, R is not H, or R is H and the compound is a salt.

With respect to the R moiety, the C₁₋₆alkyl moiety may be unsubstituted, or it may be substituted, such as with a 5- or 6-membered nonaromatic heterocyclyl, OH, —OC(O)—R^{a}, —N(R^{b})₂, —OC(O)—R^{c}, carboxyl, or a combination thereof;

the C₁₋₆acyl moiety may be unsubstituted or it may be substituted with —C(O)O—C₁₋₄alkyl, —C(O)O—C₁₋₄alkyl—N(R^{b})₂, N(R^{b})₂, —NHC(O)C₁₋₄alkyl, or a combination thereof;

the 5- or 6-membered heterocyclyl moiety may be a 5- or 6-membered oxygen-containing heterocyclyl, and/or may be substituted with hydroxyl, hydroxymethyl, or a combination thereof; or

the —C(O)O—C₁₋₆aliphatic may be —C(O)O—C₁₋₆alkyl optionally substituted with —OC(O)C₁₋₄alkyl, or N(R^{b})₂, or the —C(O)O—C₁₋₆aliphatic may be —C(O)O—C₃₋₆cycloalkyl optionally substituted with C₁₋₄alkyl.

In any embodiments, each R^{a} independently is 5-membered nonaromatic heterocyclyl, aryl substituted with —CH₂N(R^{b})₂, C₃₋₆cycloalkyl substituted with carboxyl, C₁₋₆alkoxy, unsubstituted C₁₋₆alkyl, or C₁₋₆alkyl substituted with one or more, such as 1, 2 or 3, of N(R^{b})₂, carboxyl, carboxyl ester, —OC₁₋₆acyl, —NHC(O)(NH₂)C₁₋₆alkyl, or —(OCH₂CH₂)₁₋₈N(R^{b})₂;

each R^{b} independently is H, unsubstituted C₁₋₆alkyl, C₁₋₆alkyl substituted with —N(R^{g})₂, carboxyl ester, or 5- or 6-membered nonaromatic heterocyclyl, or two R^{b} together with the nitrogen to which they are attached form a C₃₋₆nonaromatic heterocyclyl moiety optionally interrupted with one or two —O— or —N(R^{g}), where R^{g} is H or C₁₋₄alkyl; and

—OC(O)—R^{c} is derived from an amino acid where the —OC(O)— moiety of —OC(O)—R^{c} corresponds to an acid moiety on the amino acid, and R^{c} comprises —N(R^{b})₂ or a nitrogen-containing nonaromatic heterocyclyl, such as a 5- or 6-membered unsaturated nitrogen-containing heterocyclyl, for example, pyrrolidinyl. The amino acid can be any amino acid, such as a naturally occurring amino acid, and may be an amino acid selected from glycine, valine, alanine, leucine, isoleucine, methionine, phenylalanine, tryptophan, tyrosine, serine, threonine, asparagine, glutamine, arginine, histidine, lysine, aspartic acid, glutamic acid, cysteine, or proline. A person of ordinary skill in the art will understand that where the amino acid comprises one or more chiral center, all enantiomers, diastereomers and/or mixtures thereof are contemplated. For example, the amino acid may be the L-amino acid, the D-amino acid or a mixture thereof In some embodiments, the amino acid is the L-amino acid. And in certain embodiments, —OC(O)—

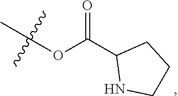 [SMILES: *OC(=O)C1CCCN1]

or —OC(O)—(CH₂)₁₋₂C(NH₂)CO₂H, where R^{d} is an amino acid side chain, and/or may be H, —CH₃, isopropyl, —CH₂CH(CH₃)₂, —CH(CH₃)Et, —CH₂CH₂SCH₃.

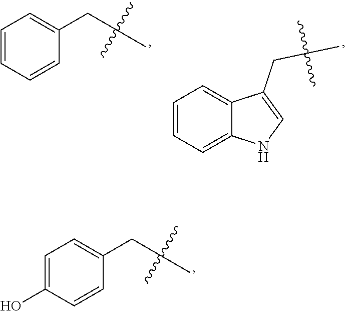 [SMILES: *Cc1ccccc1 ; *Cc1c[nH]c2ccccc12 ; *Cc1ccc(O)cc1]

—CH₂OH, —CH(OH)CH₃, —CH₂C(O)NH₂, —CH₂CH₂C(O)NH₂, —CH₂SH, —CH₂CH₂CH₂NHC(O)(NH)NH₂,

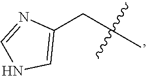 [SMILES: *Cc1c[nH]cn1]

—CH₂CH₂CH₂CH₂NH₂, —CH₂CO₂H, or CH₂CH₂CO₂H.

In any embodiments, the compound may be a salt, such as a pharmaceutically acceptable salt as defined herein, and in some embodiments, the salt is a hydrochloride, citrate, hemicitrate, hemitartrate, tartrate, benzene sulfonate, mesylate, sodium, hemisuccinate, or succinate salt.

Some exemplary compounds according to formula I include:

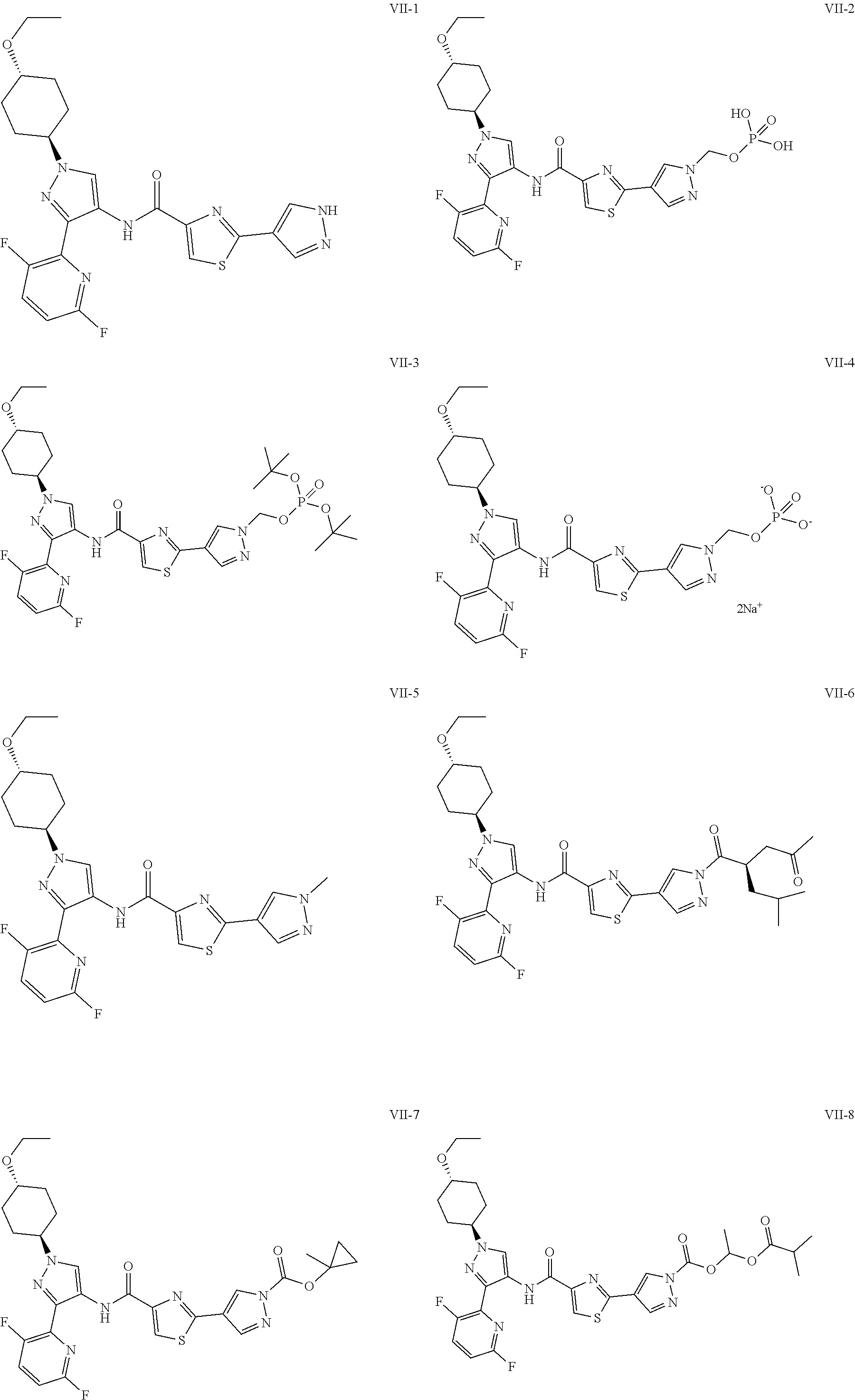

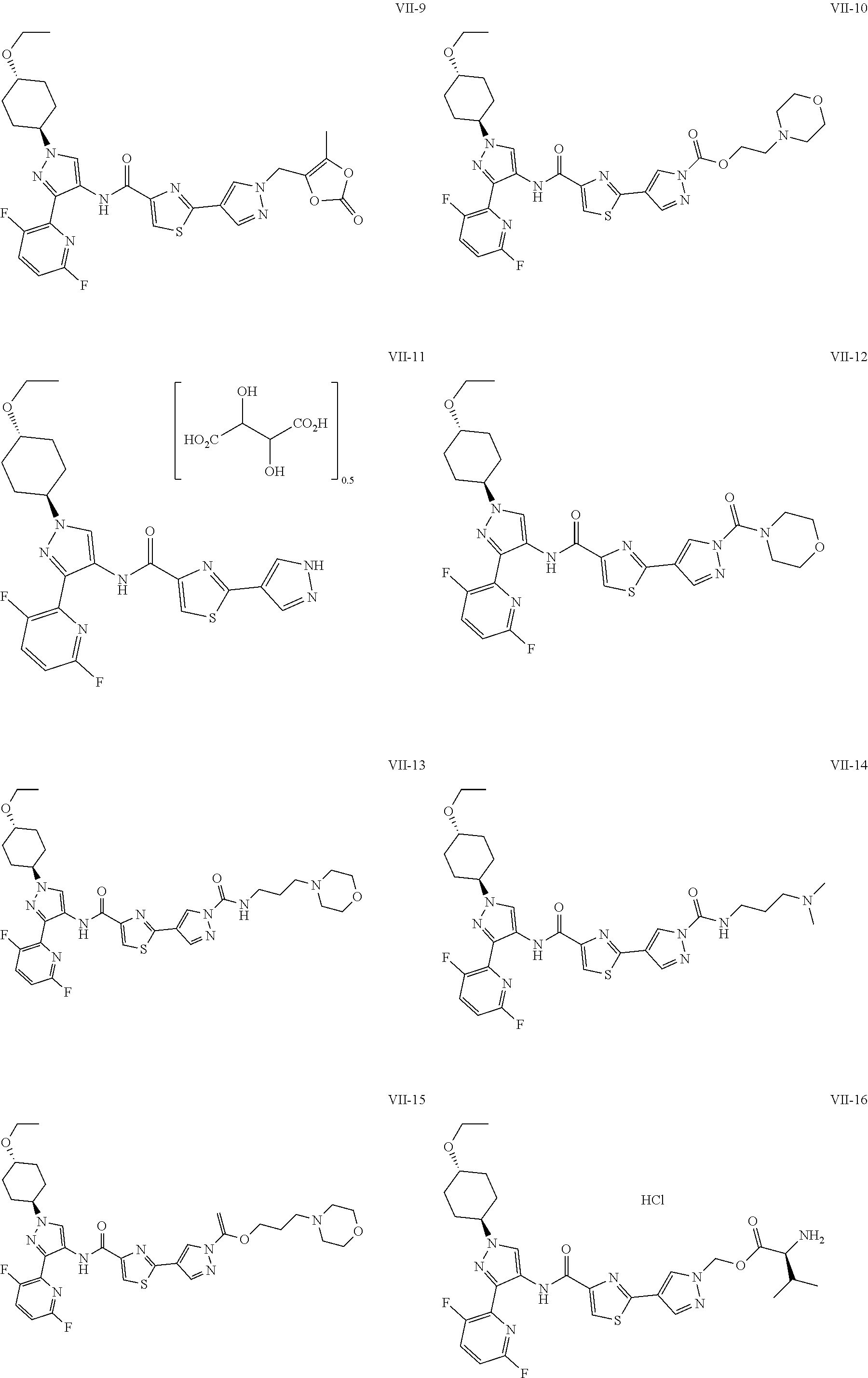

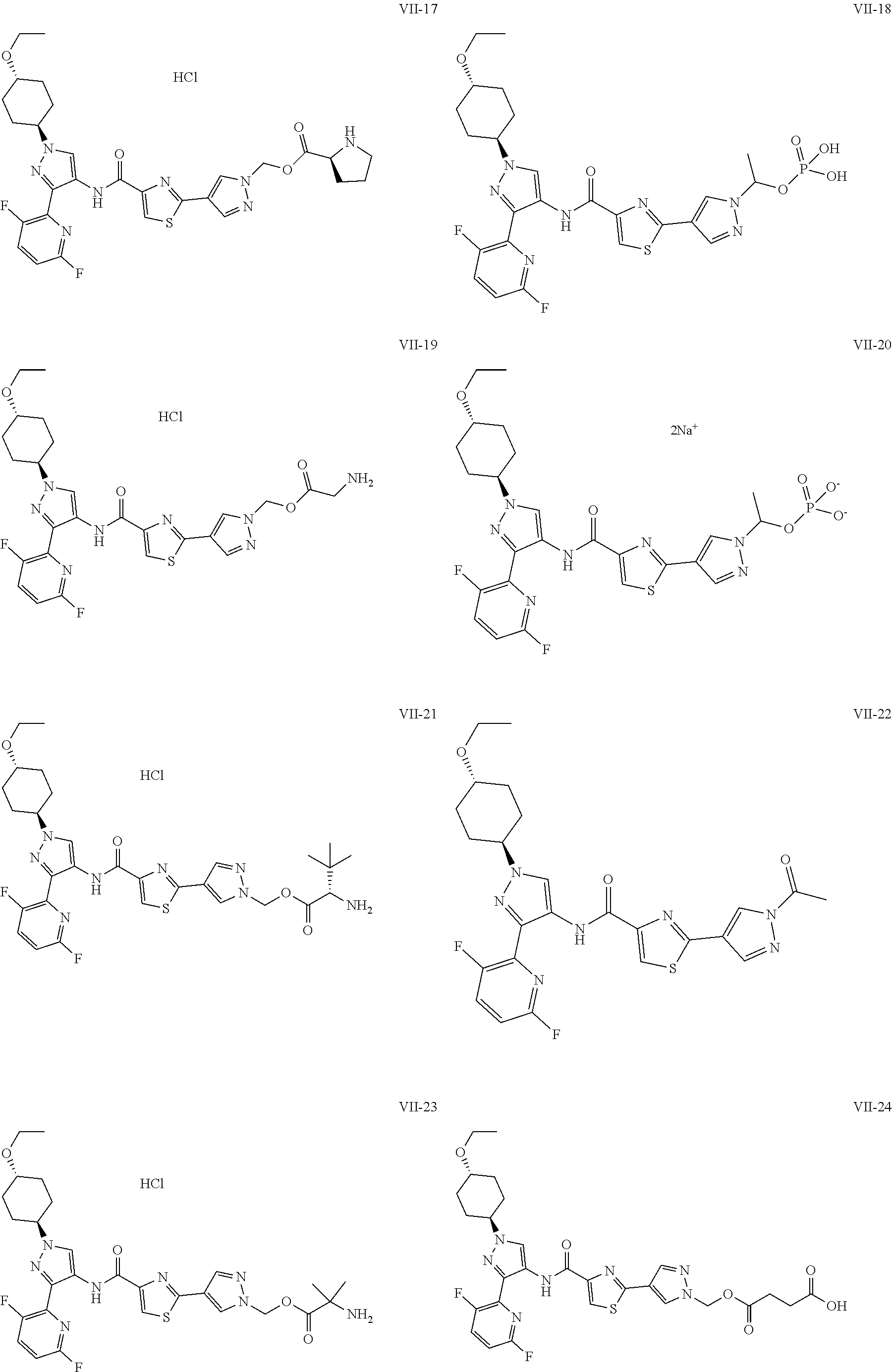

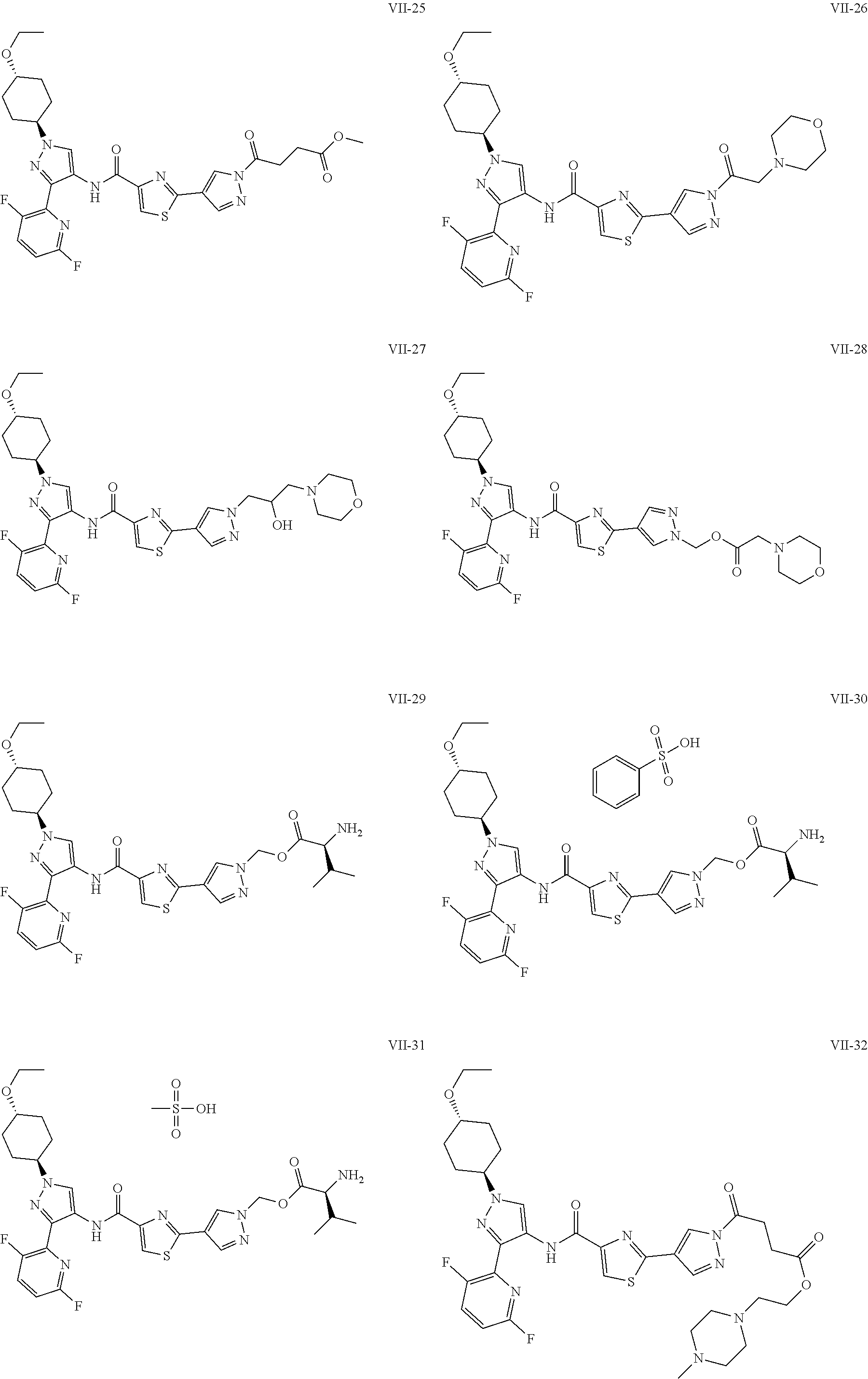

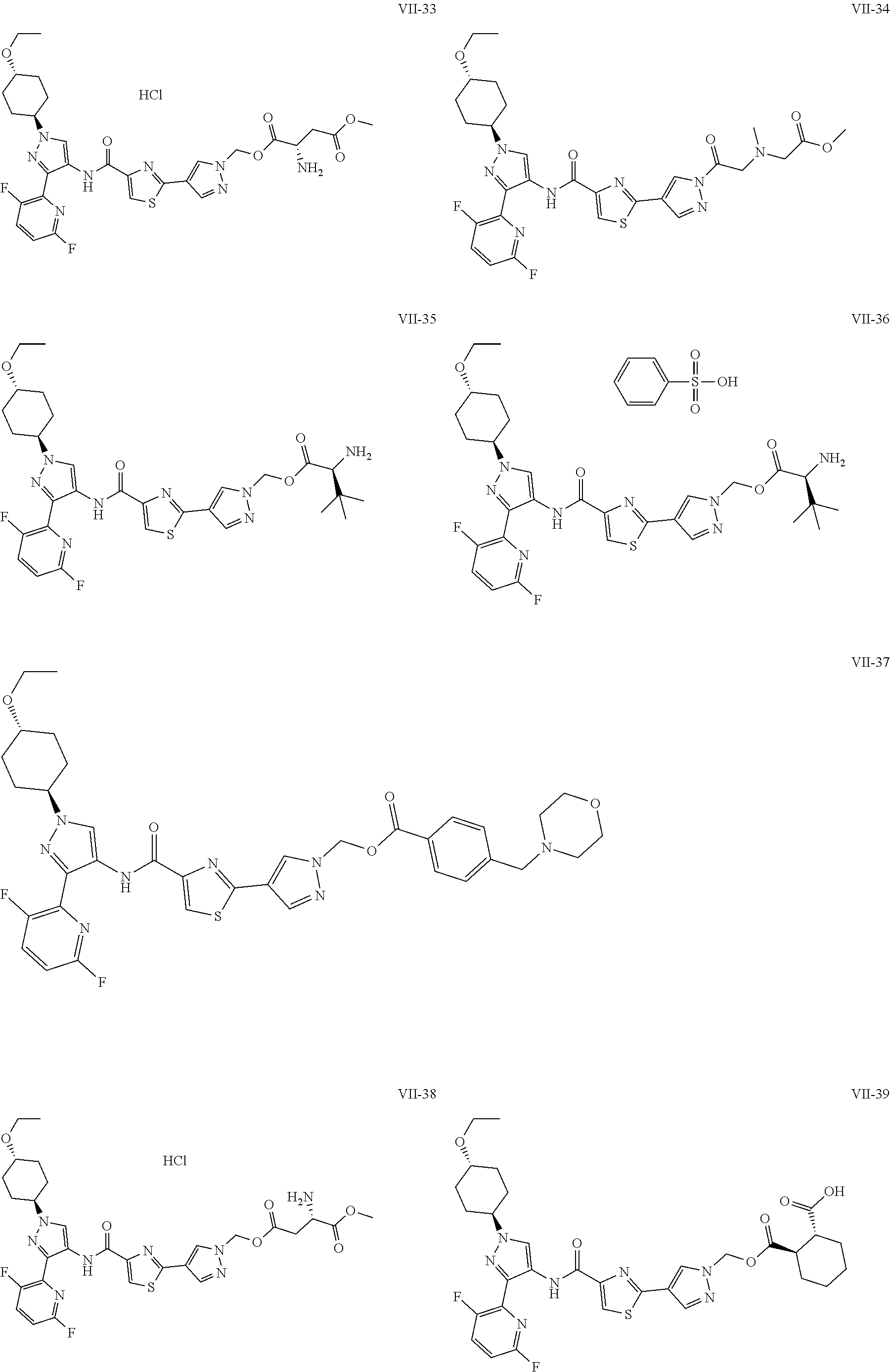

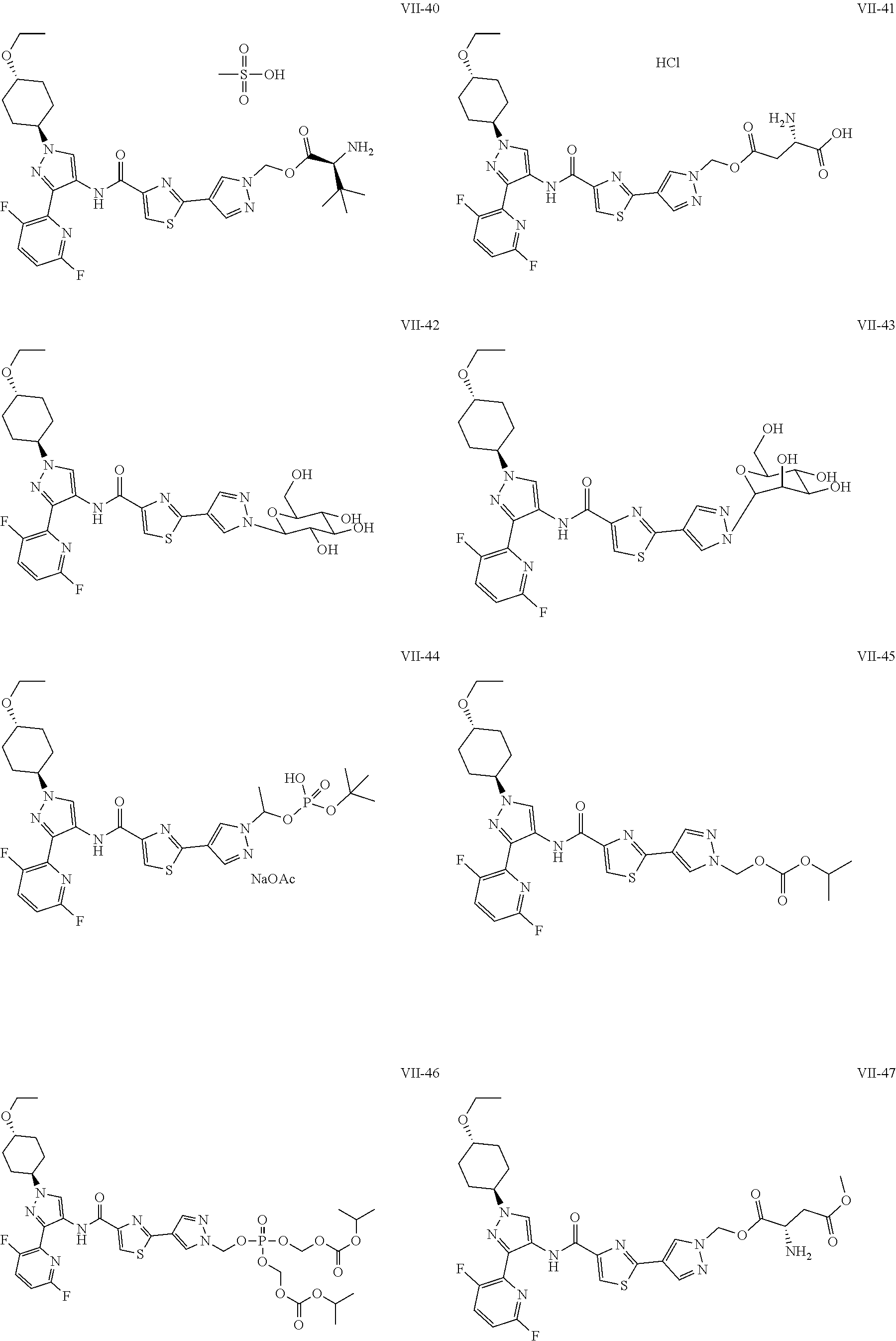

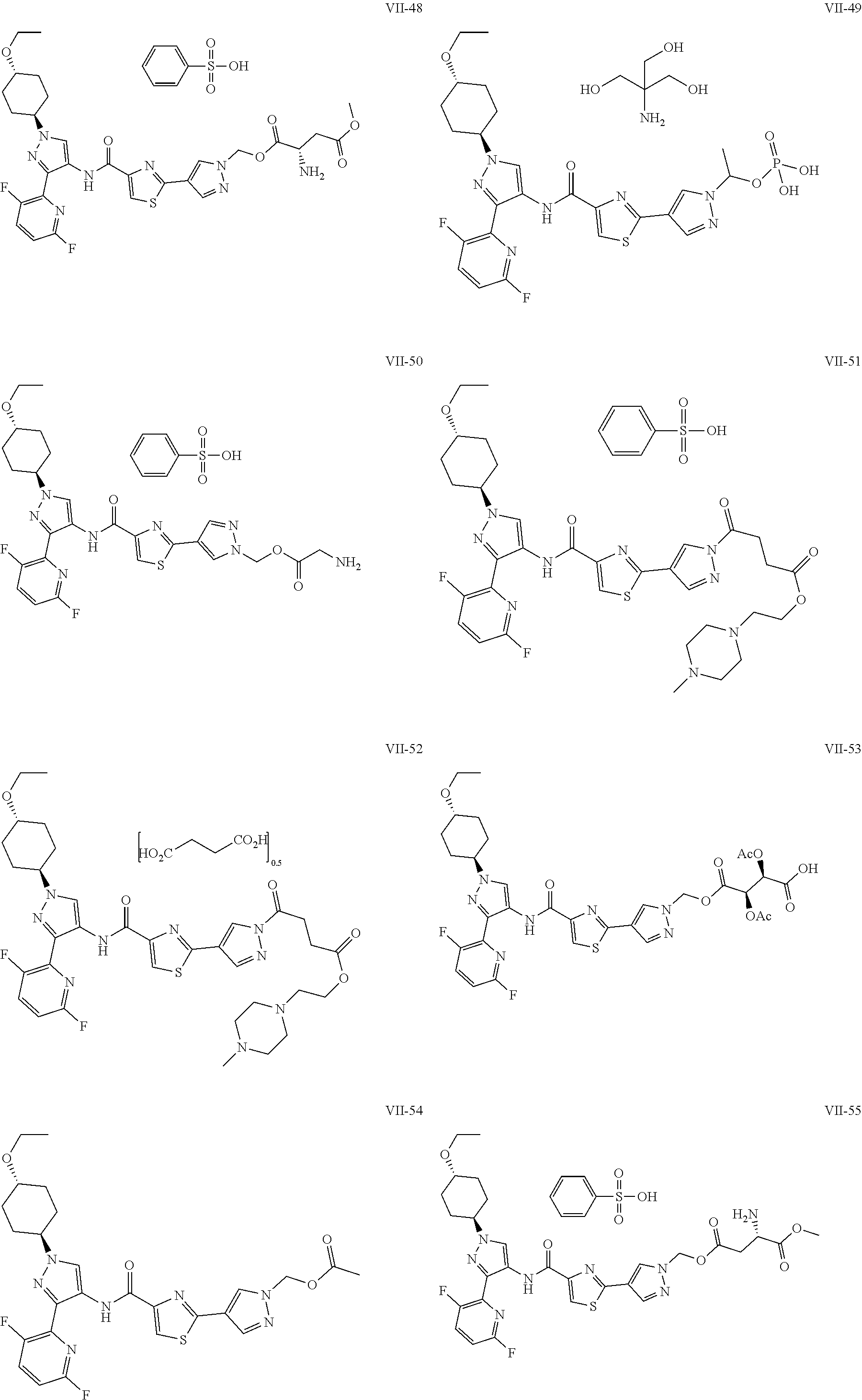

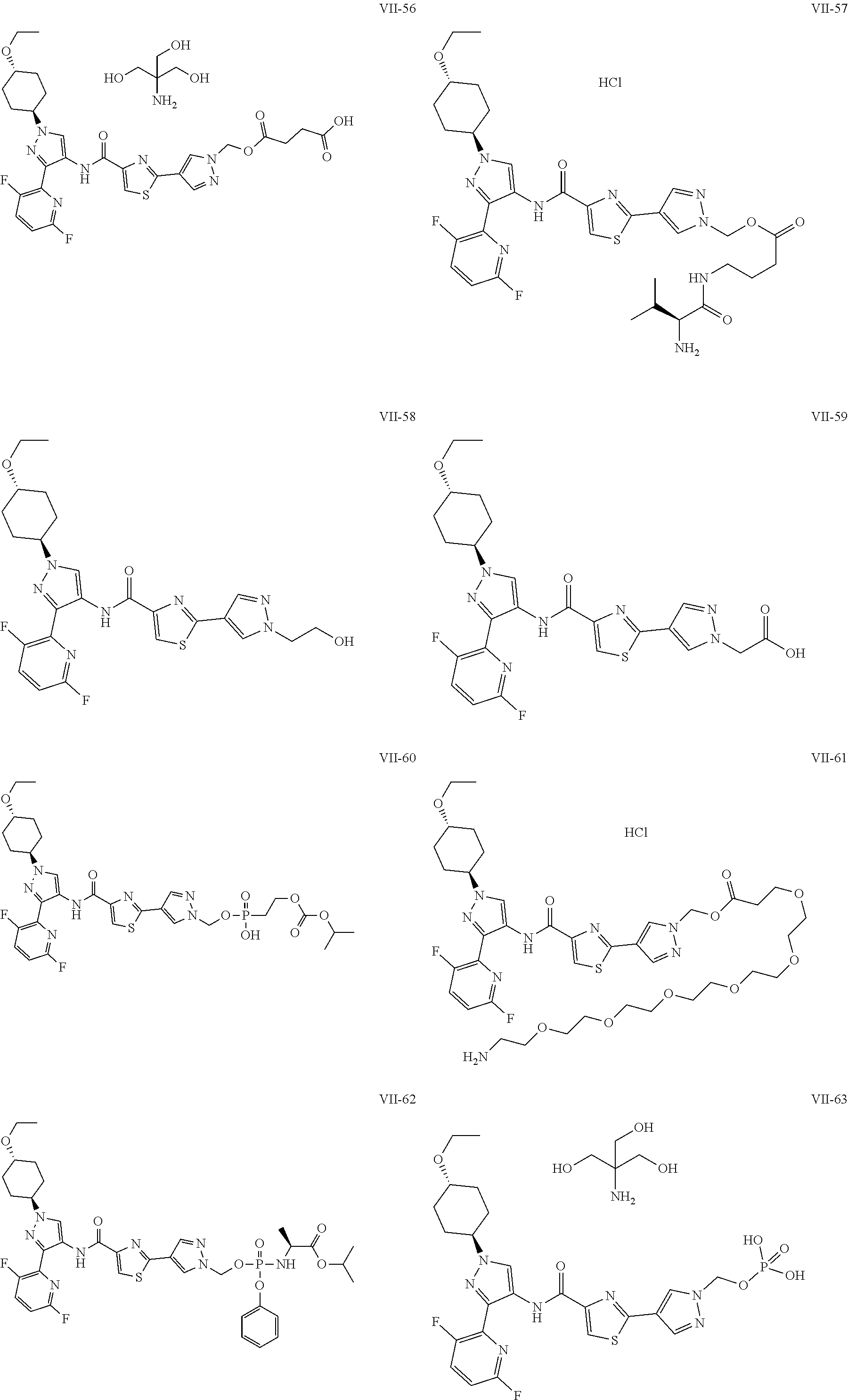

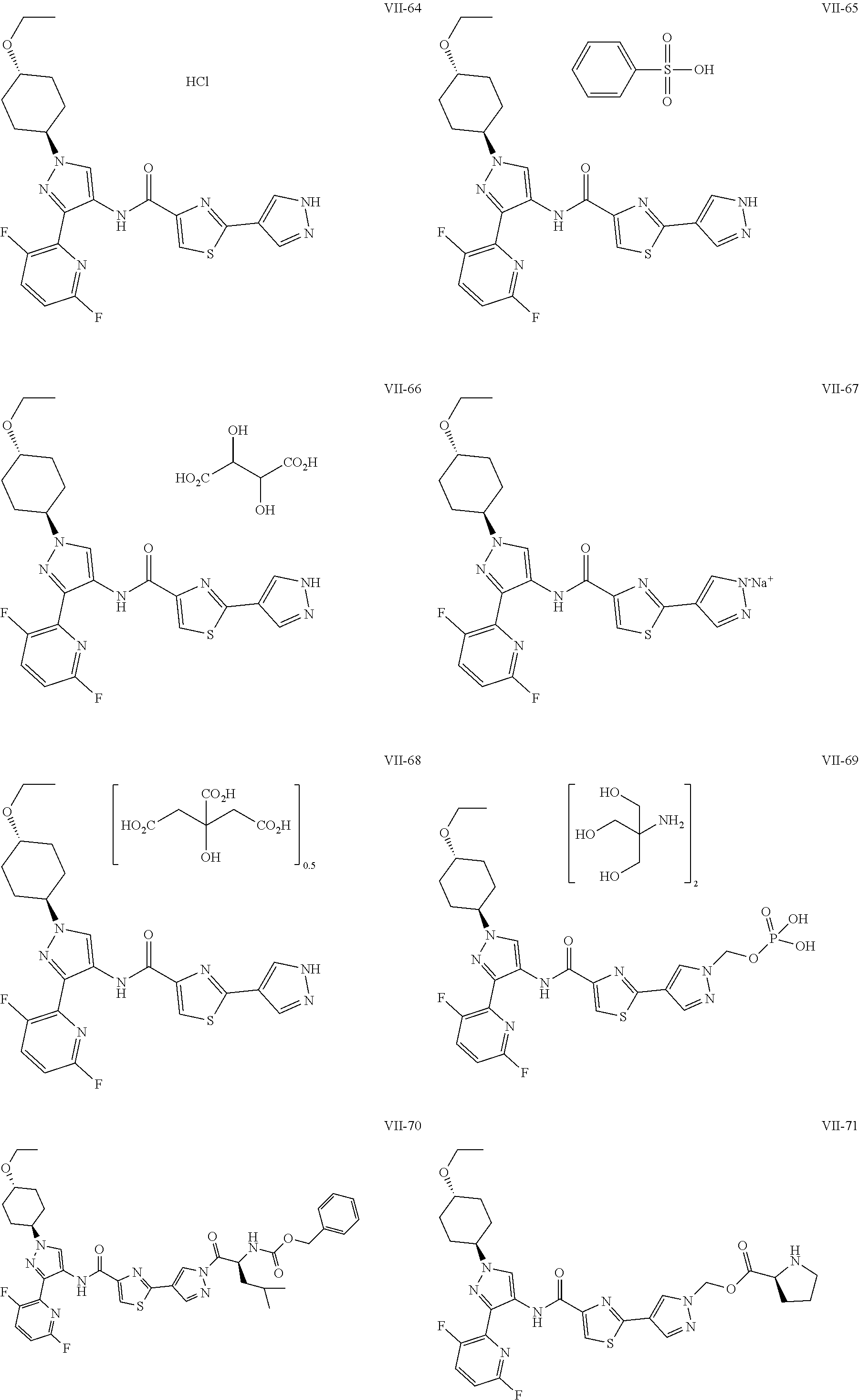

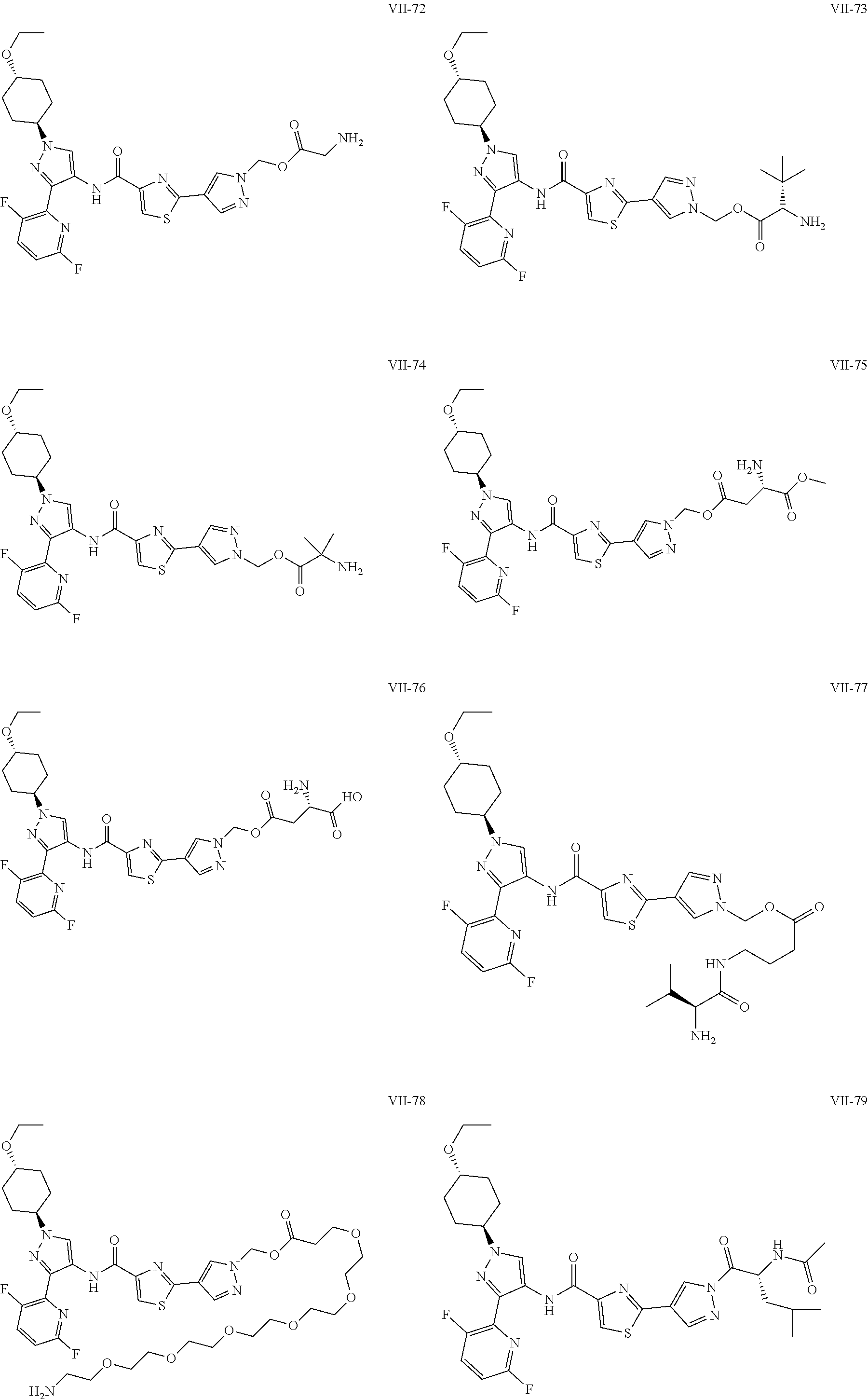

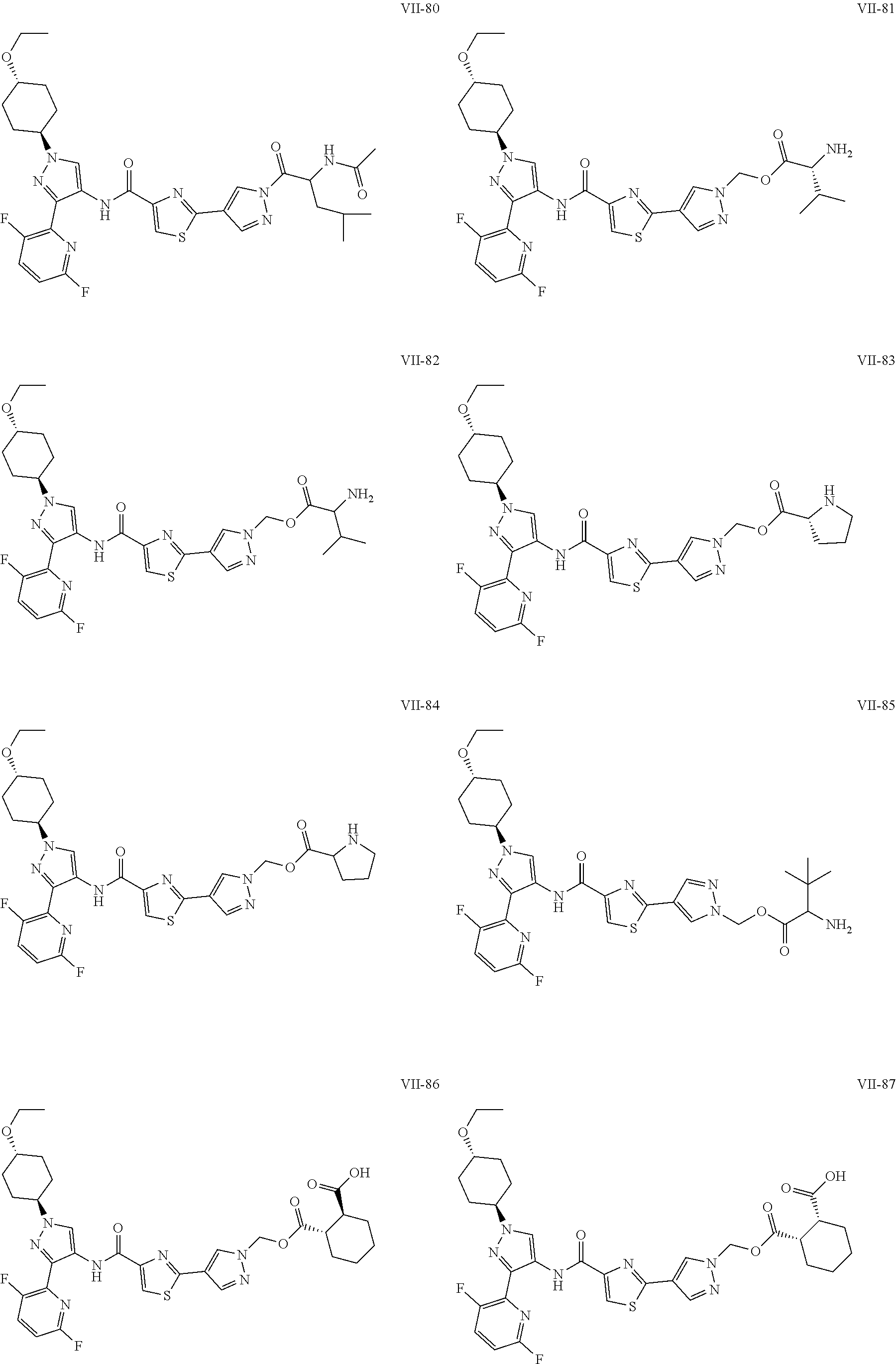

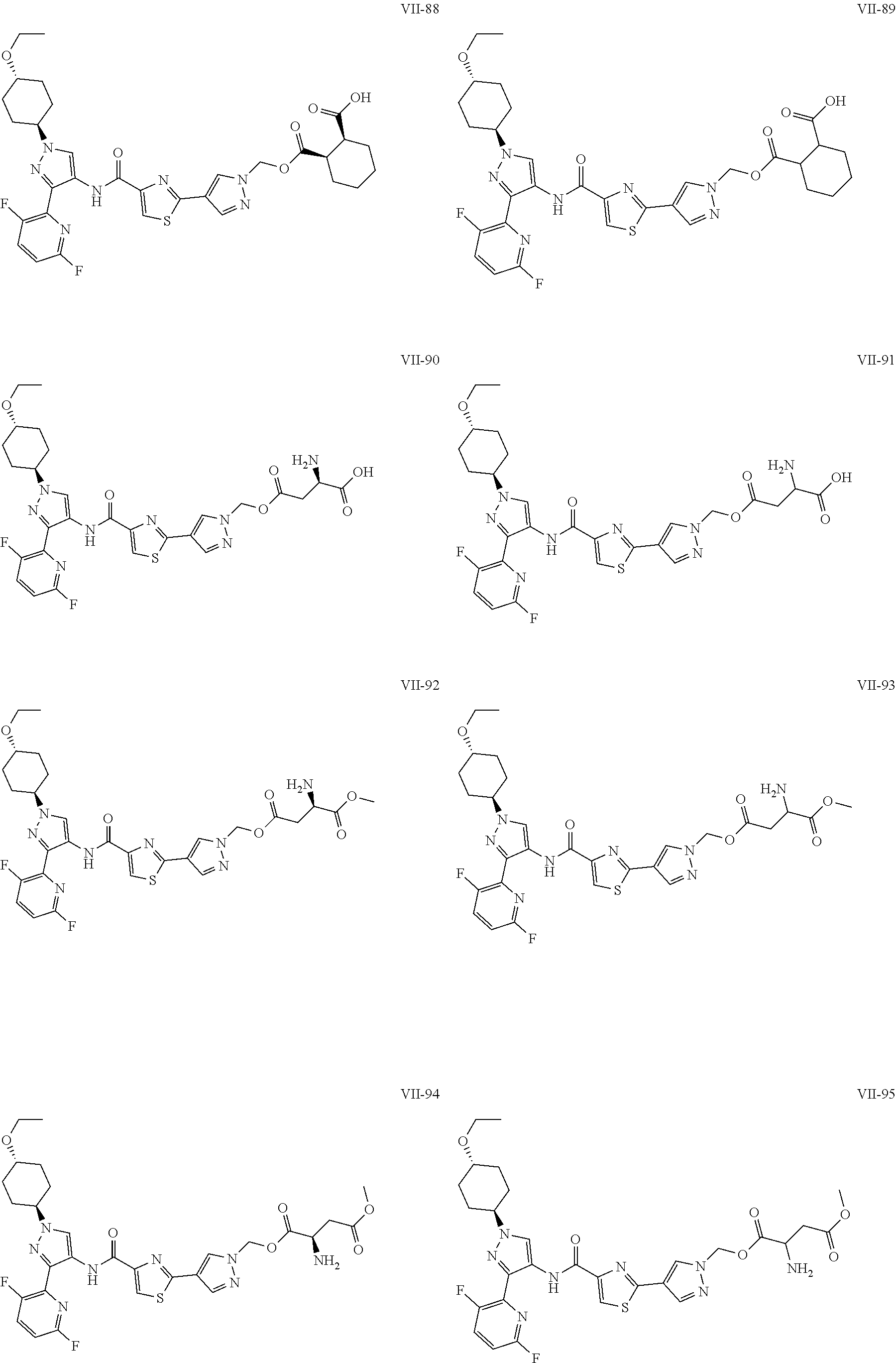

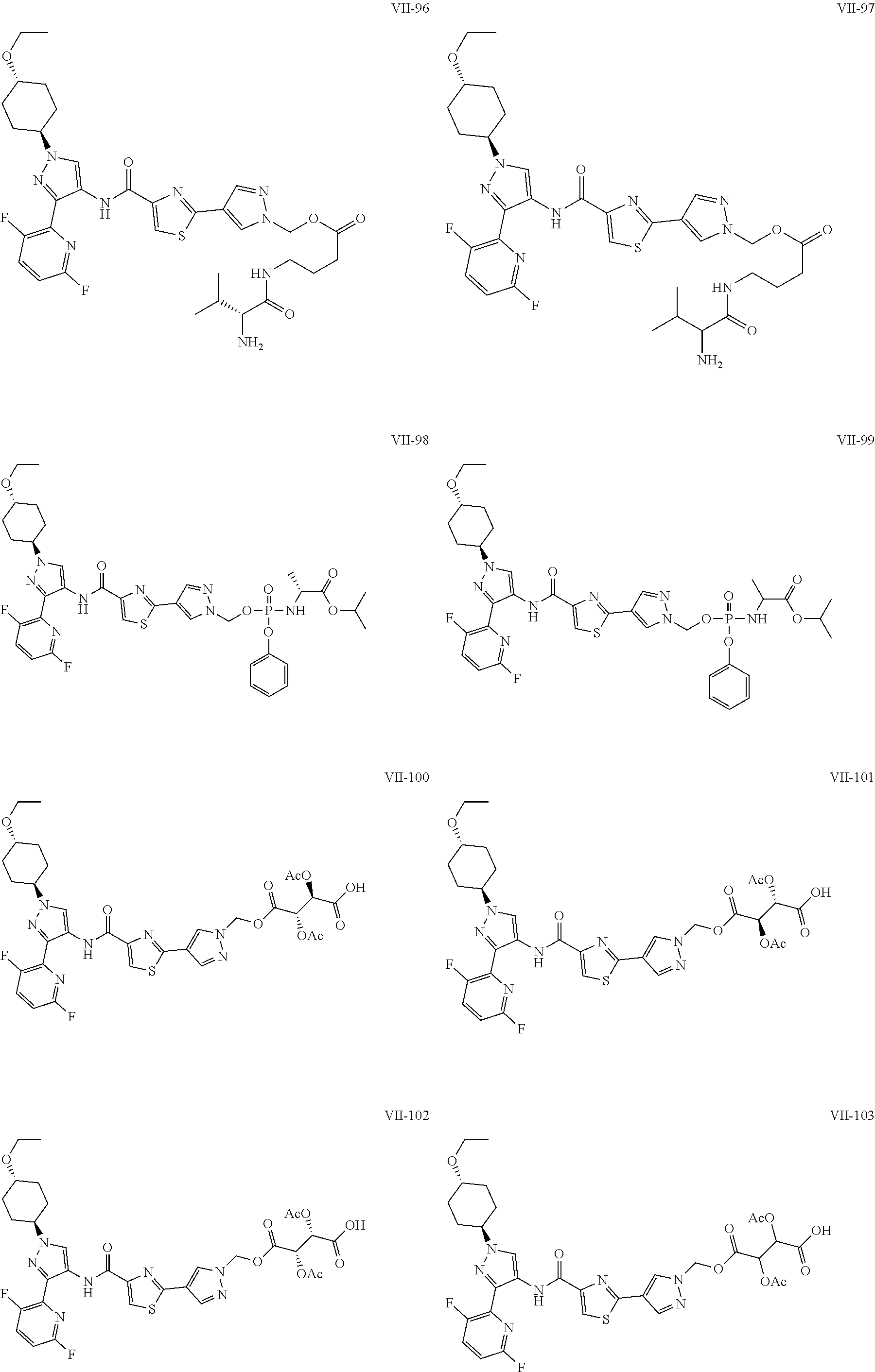

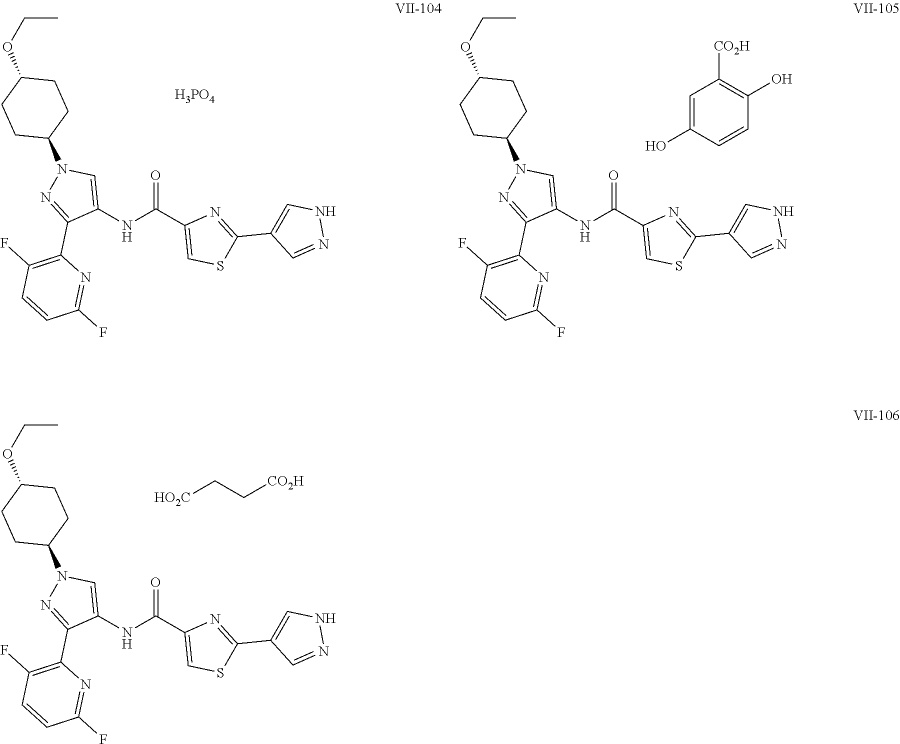 [SMILES: CCO[C@H]1CC[C@H](n2cc(NC(=O)c3csc(-c4cn[nH]c4)n3)c(-c3nc(F)ccc3F)n2)CC1 ; "H3PO4" ; CCO[C@H]1CC[C@H](n2cc(NC(=O)c3csc(-c4cn[nH]c4)n3)c(-c3nc(F)ccc3F)n2)CC1 ; O=C(O)c1cc(O)ccc1O ; CCO[C@H]1CC[C@H](n2cc(NC(=O)c3csc(-c4cn[nH]c4)n3)c(-c3nc(F)ccc3F)n2)CC1 ; O=C(O)CCC(=O)O]

Exemplary compounds according to formula I include:

VII-1: N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide;

VII-2: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl dihydrogen phosphate;

VII-3: di-tert-butyl 44-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl) phosphate;

VII-4: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl phosphate disodium salt;

VII-5: N-(3-(3,6-difluoropyridin-2-yl)-1-((1r,4r)-4-ethoxycyclohexyl)-1H-pyrazol-4-yl)-2-(1-methyl-1H-pyrazol-4-yl)thiazole-4-carboxamide;

VII-6: 2-(1-(acetyl-L-leucyl)-1H-pyrazol-4-yl)-N-(3-(3,6-difluoropyridin-2-yl)-1-((1r,4r)-4-ethoxycyclohexyl)-1H-pyrazol-4-ypthiazole-4-carboxamide;

VII-7: 1-methylcyclopropyl 4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazole-1-carboxylate;

VII-8: 1-(isobutyryloxy)ethyl 4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazole-1-carboxylate;

VII-9: N-(3-(3,6-difluoropyridin-2-yl)-1-((1r,4r)-4-ethoxycyclohexyl)-1H-pyrazol-4-yl)-2-(1-((5-methyl-2-oxo-1,3-dioxol-4-yl)methyl)-1H-pyrazol-4-yl)thiazole-4-carboxamide;

VII-10: 2-morpholinoethyl 4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazole-1-carboxylate;

VII-11: N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide hemi-tartrate salt;

VII-12: N-(3-(3,6-difluoropyridin-2-yl)-1-((1r,4r)-4-ethoxycyclohexyl)-1H-pyrazol-4-yl)-2-(1-(morpholine-4-carbonyl)-1H-pyrazol-4-yl)thiazole-4-carboxamide;

VII-13: N-(3-(3,6-difluoropyridin-2-yl)-1-((1r,4r)-4-ethoxycyclohexyl)-1H-pyrazol-4-yl)-2-(1-((3-morpholinopropyl)carbamoyl)-1H-pyrazol-4-yl)thiazole-4-carboxamide;

VII-14: N-(3-(3,6-difluoropyridin-2-yl)-1-((1r,4r)-4-ethoxycyclohexyl)-1H-pyrazol-4-yl)-2-(1-((3-(dimethylamino)propyl)carbamoyl)-1H-pyrazol-4-yl)thiazole-4-carboxamide;

VII-15: 3-morpholinopropyl 4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazole-1-carboxylate;

VII-16: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl L-valinate hydrochloride;

VII-17: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl L-prolinate hydrochloride;

VII-18: 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl dihydrogen phosphate;

VII-19: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl glycinate hydrochloride;

VII-20: 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate disodium salt;

VII-21: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl (S)-2-amino-3,3-dimethylbutanoate hydrochloride;

VII-22: 2-(1-acetyl-1H-pyrazol-4-yl)-N-(3-(3,6-difluoropyridin-2-yl)-1-((1r,4r)-4-ethoxycyclohexyl)-1H-pyrazol-4-yl)thiazole-4-carboxamide;

VII-23: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 2-amino-2-methylpropanoate hydrochloride;

VII-24: 4-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)-4-oxobutanoic acid;

VII-25: methyl 4-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)-4-oxobutanoate;

VII-26: N-(3-(3,6-difluoropyridin-2-yl)-1-((1r,4r)-4-ethoxycyclohexyl)-1H-pyrazol-4-yl)-2-(1-(2-morpholinoacetyl)-1H-pyrazol-4-yl)thiazole-4-carboxamide;

VII-27: N-(3-(3,6-difluoropyridin-2-yl)-1-((1r,4r)-4-ethoxycyclohexyl)-1H-pyrazol-4-yl)-2-(1-(2-hydroxy-3-morpholinopropyl)-1H-pyrazol-4-ypthiazole-4-carboxamide;

VII-28: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 2-morpholinoacetate;

VII-29: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl L-valinate;

VII-30: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl L-valinate benzene sulfonate;

VII-31: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl L-valinate mesylate;

VII-32: 2-(4-methylpiperazin-1-yl)ethyl 4-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)-4-oxobutanoate;

VII-33: 1-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl) 4-methyl L-aspartate hydrochloride;

VII-34: methyl N-(2-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)-2-oxoethyl)-N-methylglycinate;

VII-35: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl (S)-2-amino-3,3-dimethylbutanoate;

VII-36: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl (S)-2-amino-3,3-dimethylbutanoate benzene sulfonate;

VII-37: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 4-(morpholinomethyl)benzoate;

VII-38: 4-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl) 1-methyl L-aspartate hydrochloride;

VII-39: (1R,2R)-2-(((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)carbonyl)cyclohexane-1-carboxylic acid;

VII-40: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl (S)-2-amino-3,3-dimethylbutanoate mesylate;

VII-41: (S)-2-amino-4-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)-4-oxobutanoic acid hydrochloride;

VII-42: N-(3-(3,6-difluoropyridin-2-yl)-1-((1r,4S)-4-ethoxycyclohexyl)-1H-pyrazol-4-yl)-2-(1-((2S,3S,4R,5R,6S)-3,4,5-trihydroxy-6-(hydroxymethyptetrahydro-2H-pyran-2-yl)-1H-pyrazol-4-yl)thiazole-4-carboxamide;

VII-43: N-(3-(3,6-difluoropyridin-2-yl)-1-((1r,4R)-4-ethoxycyclohexyl)-1H-pyrazol-4-yl)-2-(1-((2R,3R,4R,5R,6S)-3,4,5-trihydroxy-6-(hydroxymethyptetrahydro-2H-pyran-2-yl)-1H-pyrazol-4-yl)thiazole-4-carboxamide;

VII-44: tert-butyl (1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl) hydrogen phosphate sodium acetate salt;

VII-45: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl isopropyl carbonate;

VII-46: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl di(((isopropoxycarbonyl)oxy)methyl) phosphate;

VII-47: 1-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl) 4-methyl L-aspartate;

VII-48: 1-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl) 4-methyl L-aspartate benzene sulfonate;

VII-49: 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl dihydrogen phosphate tris salt;

VII-50: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl glycinate benzene sulfonate;

VII-51: 2-(4-methylpiperazin-1-yl)ethyl 4-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)-4-oxobutanoate benzene sulfonate;

VII-52: 2-(4-methylpiperazin-1-yl)ethyl 4-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)-4-oxobutanoate succinate salt;

VII-53: (2R,3R)-2,3-diacetoxy-4-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)-4-oxobutanoic acid;

VII-54: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl acetate;

VII-55: 4-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl) 1-methyl L-aspartate benzene sulfonate;

VII-56: 4-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)-4-oxobutanoic acid tris salt;

VII-57: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 4-((S)-2-amino-3-methylbutanamido)butanoate hydrochloride;

VH-58: N-(3-(3,6-difluoropyridin-2-yl)-1-((1r,4r)-4-ethoxycyclohexyl)-1H-pyrazol-4-yl)-2-(1-(2-hydroxyethyl)-1H-pyrazol-4-yl)thiazole-4-carboxamide;

VII-59: 2-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)acetic acid;

VII-60: ((((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)(hydroxy)phosphorypoxy)methyl isopropyl carbonate;

VII-61: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 1-amino-3,6,9,12,15,18-hexaoxahenicosan-21-oate hydrochloride;

VII-62: isopropyl (((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)(phenoxy)phosphoryl)-L-alaninate;

VII-63: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl dihydrogen phosphate tris salt;

VII-64: N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide hydrochloride;

VII-65: N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide benzene sulfonate;

VII-66: N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide tartrate;

VII-67: N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide sodium salt;

VII-68: N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide hemicitrate;

VII-69: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl dihydrogen phosphate ditris salt;

VII-70: benzyl ((S)-1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)-4-methyl-1-oxopentan-2-yl)carbamate;

VII-71: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl L-prolinate;

VII-72: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl glycinate;

VII-73: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl (R)-2-amino-3,3-dimethylbutanoate;

VII-74: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 2-amino-2-methylpropanoate;

VII-75: 4-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl) 1-methyl L-aspartate;

VII-76: (S)-2-amino-4-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)-4-oxobutanoic acid;

VII-77: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 4-((S)-2-amino-3-methylbutanamido)butanoate;

VII-78: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 1-amino-3,6,9,12,15,18-hexaoxahenicosan-21-oate;

VII-79: 2-(1-(acetyl-D-leucyl)-1H-pyrazol-4-yl)-N-(3-(3,6-difluoropyridin-2-yl)-1-((1r,4r)-4-ethoxycyclohexyl)-1H-pyrazol-4-ypthiazole-4-carboxamide;

VII-80: 2-(1-(acetylleucyl)-1H-pyrazol-4-yl)-N-(3-(3,6-difluoropyridin-2-yl)-1-((1r,4r)-4-ethoxycyclohexyl)-1H-pyrazol-4-ypthiazole-4-carboxamide;

VII-81: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl D-valinate;

VII-82: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl valinate;

VII-83: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl D-prolinate;

VII-84: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl prolinate;

VII-85: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 2-amino-3,3-dimethylbutanoate;

VII-86: (1S,2S)-2-(((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)carbonyl)cyclohexane-1-carboxylic acid;

VII-87: (1R,2S)-2-(((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)carbonyl)cyclohexane-1-carboxylic acid;

VII-88: (1S,2R)-2-(((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)carbonyl)cyclohexane-1-carboxylic acid;

VII-89: 2-(((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)carbonyl)cyclohexane-1-carboxylic acid;

VII-90: (R)-2-amino-4-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)-4-oxobutanoic acid;

VII-91: 2-amino-4-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)-4-oxobutanoic acid;

VII-92: 4-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl) 1-methyl D-aspartate;

VII-93: 4-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl) 1-methyl aspartate;

VII-94: 1-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl) 4-methyl D-aspartate;

VII-95: 1-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl) 4-methyl aspartate;

VII-96: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 4-((R)-2-amino-3-methylbutanamido)butanoate;

VII-97: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 4-(2-amino-3-methylbutanamido)butanoate;

VII-98: isopropyl (((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)(phenoxy)phosphoryl)-D-alaninate;

VII-99: isopropyl (((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)(phenoxy)phosphorypalaninate;

VII-100: (2R,3S)-2,3-diacetoxy-4-((4-(4-((3-(3,6-difluoropyridin-2-yl)-1-((1r,4r)-4-thoxycyclohexyl)-1H-pyrazol-4-yl)carbamoyl)thiazol-2-yl)-1H-pyrazol-1-yl)methoxy)-4-oxobutanoic acid;

VII-101: (2S,3R)-2,3-diacetoxy-4-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)-4-oxobutanoic acid;

VII-102: (2S,3S)-2,3-diacetoxy-4-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)-4-oxobutanoic acid;

VII-103: 2,3-diacetoxy-4-((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)-4-oxobutanoic acid;

VII-104: N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide phosphate;

VII-105: N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide gentisate; or

VII-106: N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide succinate.

### III. Synthesis

A. Synthesis of the Pyrimidine Diamine Compounds According to Formula I

The 2,4-pyrimidinediamine compounds described herein can be synthesized via a variety of different synthetic routes using commercially available starting materials and/or starting materials prepared by conventional synthetic methods. Suitable exemplary methods that can be routinely adapted to synthesize the 2,4-pyrimidinediamine compounds and prodrugs described herein are found in U.S. Pat. No. 5,958,935, the disclosure of which is incorporated herein by reference. Specific examples describing the synthesis of numerous 2,4-pyrimidinediamine compounds and prodrugs, as well as intermediates thereof, are described in U.S. application Ser. No. 10/355,543, filed Jan. 31, 2003 (US2004/0029902A1), the contents of which are incorporated herein by reference. Suitable exemplary methods that can be routinely used and/or adapted to synthesize active 2,4-substituted pyrimidine diamine compounds can also be found in international application Serial No. PCT/US03/03022 filed Jan. 31, 2003 (WO 03/063794), U.S. application Ser. No. 10/631,029 filed Jul. 29, 2003, international application Serial No. PCT/US03/24087 (WO2004/014382), U.S. application Ser. No. 10/903,263 filed Jul. 30, 2004, and international application Serial No. PCT/US2004/24716 (WO005/016893), the disclosures of which are incorporated herein by reference. All of the compounds described herein (including prodrugs) can be prepared by routine adaptation of these methods.

Specific exemplary synthetic methods for the 2,4-substituted pyrimidinediamines described herein are also described in Example 1, below. Those of skill in the art will also be able to readily adapt these examples for the synthesis of additional 2,4-substituted pyrimidinediamines as described herein.

A variety of exemplary synthetic routes that can be used to synthesize the 2,4-pyrimidinediamine compounds described herein are depicted in Schemes (I)-(VII), below. These methods can be routinely adapted to synthesize the 2,4-substituted pyrimidinediamine compounds described herein. After each reaction step, the product can be purified or can, depending on the chemistry, be used in the next step without purification.

For example, the compounds can be synthesized from substituted or unsubstituted uracils as illustrated in Scheme (I), below. In Scheme (I), ring A, R⁵, (R²)ₚ, X, Y, Z¹, and Z² are as defined herein for pyrimidine diamine compounds. According to Scheme (I), uracil A-1 is dihalogenated at the 2- and 4-positions using a standard halogenating agent such as POCl₃ (or other standard halogenating agent) under standard conditions to yield 2,4-dichloropyrimidine A-2. Depending upon the R⁵ substituent, in pyrimidinediamine A-2, the chloride at the C4 position is more reactive towards nucleophiles than the chloride at the C2 position. This differential reactivity can be exploited to synthesize 2,4-pyrimidinediamines I by first reacting 2,4-dichloropyrimidine A-2 with one equivalent of amine A-3, yielding 4N-substituted-2-chloro-4-pyrimidineamine A-4, followed by amine A-5 to yield a 2,4-pyrimidinediamine of formula A-6 (compounds of formula I, where each of R³ and R⁴ are H). Compounds of formula I, where either or both of the NH groups at C2 and C4 of the pyrimidine are substituted, can be made, e.g., via alkylation of the NH groups.

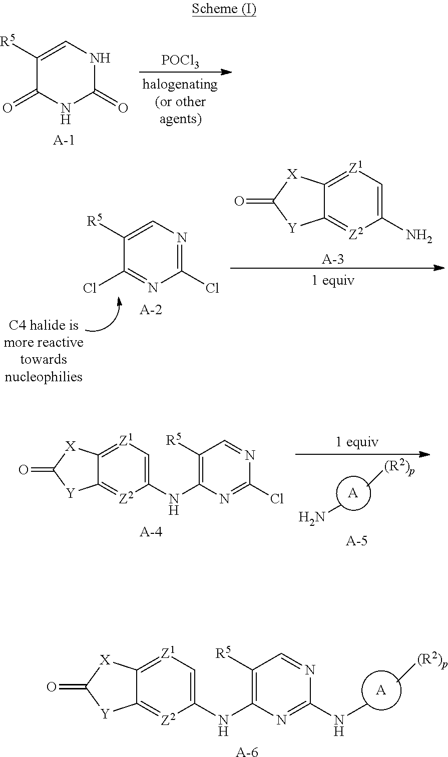

Typically, the C4 halide is more reactive towards nucleophiles, as illustrated in the Scheme. However, as will be recognized by skilled artisans, the identity of the R⁵ substituent may alter this reactivity. For example, when R⁵ is trifluoromethyl, a 50:50 mixture of 4N-substituted-4-pyrimidineamine A-4 and the corresponding 2N-substituted-2-pyrimidineamine is obtained. The regioselectivity of the reaction can also be controlled by adjusting the solvent and other synthetic conditions (such as temperature), as is well-known in the art.

The reactions depicted in Scheme (I) may proceed more quickly when the reaction mixtures are heated via microwave. When heating in this fashion, the following conditions can be used: heat to 175° C. in ethanol for 5-20 min. in a Smith Reactor (Personal Chemistry, Uppsala, Sweden) in a sealed tube (at 20 bar pressure).

The uracil A-1 starting materials can be purchased from commercial sources or prepared using standard techniques of organic chemistry. Commercially available uracils that can be used as starting materials in Scheme (I) include, by way of example and not limitation, uracil (Aldrich #13,078-8; CAS Registry 66-22-8); 5-bromouracil (Aldrich #85,247-3; CAS Registry 51-20-7; 5-fluorouracil (Aldrich #85,847-1; CAS Registry 51-21-8); 5-iodouracil (Aldrich #85,785-8; CAS Registry 696-07-1); 5-nitrouracil (Aldrich #85,276-7; CAS Registry 611-08-5); 5-(trifluoromethyl)-uracil (Aldrich #22,327-1; CAS Registry 54-20-6). Additional 5-substituted uracils are available from General Intermediates of Canada, Inc., Edmonton, Calif. and/or Interchim, Cedex, France, or can be prepared using standard techniques. Myriad textbook references teaching suitable synthetic methods are provided infra.

Amines A-3 and A-5 can be purchased from commercial sources or, alternatively, can be synthesized utilizing standard techniques. For example, suitable amines can be synthesized from nitro precursors using standard chemistry. Specific exemplary reactions are provided in the Examples section. See also Vogel, 1989, Practical Organic Chemistry, Addison Wesley Longman, Ltd. and John Wiley & Sons, Inc.

A person of ordinary skill in the art will recognize that in some instances, amines A-3 and A-5 and/or substituent X on uracil A-1 can include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Green & Wuts.

Thus, protecting group refers to a group of atoms that, when attached to a reactive functional group in a molecule, mask, reduce or prevent the reactivity of the functional group. Typically, a protecting group can be selectively removed as desired during the course of a synthesis. Examples of protecting groups can be found in Green & Wuts and in Harrison et al., Compendium of Synthetic Organic Methods, Vols. 1-8, 1971-1996, John Wiley & Sons, NY. Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated to form acetate and benzoate esters or alkylated to form benzyl and trityl ethers, as well as alkyl ethers, tetrahydropyranyl ethers, trialkylsilyl ethers (e.g., TMS or TIPPS groups) and allyl ethers.

A specific embodiment of Scheme (I) utilizing 5-fluorouracil (Aldrich #32,937-1) as a starting material is illustrated in Scheme (Ia), below. In Scheme (Ia), ring A, (R²)ₚ, X, Y, Z¹, and Z² are as previously defined for Scheme (I). Compound A-10, a 2N,4N-disubstituted-5-fluoro-2,4-pyrimidinediamine, can be obtained by reacting 2,4-dichloro-5-fluoropyrimidine A-8 (commercially available or made from A-7 as depicted e.g. starting with a uracil and dehydrohalogenating with e.g. POCl₃) with, optimally, one equivalent of amine A-3 to yield 2-chloro-N4-substituted-5-fluoro-4-pyrimidineamine A-9 followed by reaction with one or more equivalents of amine A-5, typically between about 1.1 equivalents of A-5 and about 2 equivalents of A-5.

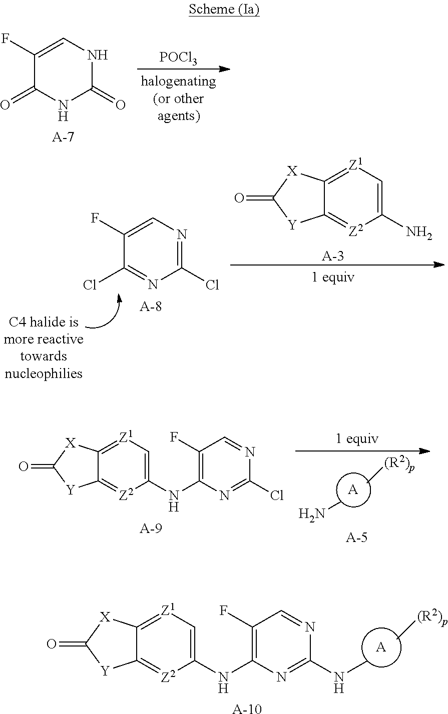

Although many of the synthetic schemes discussed above do not illustrate the use of protecting groups, skilled artisans will recognize that in some instances certain substituents, such as, for example, R² and/or other groups, can include functionality requiring protection. The exact identity of the protecting group used will depend upon, among other things, the identity of the functional group being protected and the reaction conditions used in the particular synthetic scheme, and will be apparent to those of skill in the art. Guidance for selecting protecting groups, their attachment and removal suitable for a particular application can be found, for example, in Green & Wuts.

Prodrugs as described herein can be prepared by routine modification of the above-described methods. Alternatively, such prodrugs can be prepared by reacting a suitably protected 2,4-pyrimidinediamine with a suitable reagent to append the desired progroup. Conditions for carrying out such reactions and for deprotecting the product to yield a prodrug as described herein are well-known.

Myriad references teaching methods useful for synthesizing pyrimidines generally, as well as starting materials described in Schemes (I)—(VII), are known in the art. For specific guidance, the reader is referred to Brown, D. J., “The Pyrimidines”, in The Chemistry of Heterocyclic Compounds, Volume 16 (Weissberger, A., Ed.), 1962, Interscience Publishers, (A Division of John Wiley & Sons), New York (“Brown I”); Brown, D. J., “The Pyrimidines”, in The Chemistry of Heterocyclic Compounds, Volume 16, Supplement I (Weissberger, A. and Taylor, E. C., Ed.), 1970, Wiley-Interscience, (A Division of John Wiley & Sons), New York (Brown II″); Brown, D. J., “The Pyrimidines”, in The Chemistry of Heterocyclic Compounds, Volume 16, Supplement II (Weissberger, A. and Taylor, E. C., Ed.), 1985, An Interscience Publication (John Wiley & Sons), New York (“Brown III”); Brown, D. J., “The Pyrimidines” in The Chemistry of Heterocyclic Compounds, Volume 52 (Weissberger, A. and Taylor, E. C., Ed.), 1994, John Wiley & Sons, Inc., New York, pp. 1-1509 (Brown IV″); Kenner, G. W. and Todd, A., in Heterocyclic Compounds, Volume 6, (Elderfield, R. C., Ed.), 1957, John Wiley, New York, Chapter 7 (pyrimidines); Paquette, L. A., Principles of Modern Heterocyclic Chemistry, 1968, W. A. Benjamin, Inc., New York, pp. 1-401 (uracil synthesis pp. 313, 315; pyrimidinediamine synthesis pp. 313-316; amino pyrimidinediamine synthesis pp. 315); Joule, J. A., Mills, K. and Smith, G. F., Heterocyclic Chemistry, 3r^{d} Edition, 1995, Chapman and Hall, London, UK, pp. 1-516; Vorbruggen, H. and Ruh—Pohlenz, C., Handbook of Nucleoside Synthesis, John Wiley & Sons, New York, 2001, pp. 1-631 (protection of pyrimidines by acylation pp. 90-91; silylation of pyrimidines pp. 91-93); Joule, J. A., Mills, K. and Smith, G. F., Heterocyclic Chemistry, 4^{th} Edition, 2000, Blackwell Science, Ltd, Oxford, UK, pp. 1-589; and Comprehensive Organic Synthesis, Volumes 1-9 (Trost, B. M. and Fleming, I., Ed.), 1991, Pergamon Press, Oxford, UK.

B. Synthesis of Compounds B-I and B-II

Compounds and as well as exemplary salts B-III to B-VII, are synthesized as described below or by analogy to the syntheses described below. Alternative syntheses would be appreciated by one of ordinary skill in the art.

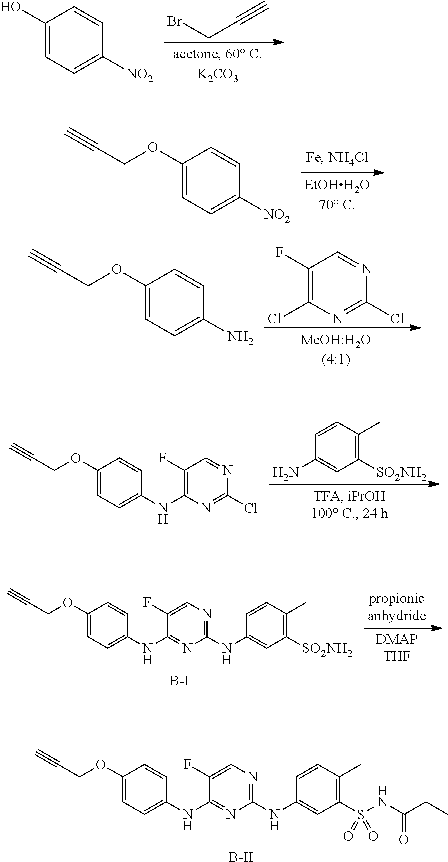

B-I: N2-(3-Aminosulfonyl-4-methylphenyl)-5-fluoro-N4-[4-(prop-2-ynyloxy)phenyl]-2,4-pyrimidinediamine

4-Nitrophenol (1.00 g, 7.19 mmol), propargyl bromide (80 wt % in toluene; 0.788 mL, 7.09 mmol), and K2CO₃ (1.08 g, 7.84 mmol) were combined and stirred in acetone (16.0 mL) at 60° C. for 18 h. The reaction mixture was cooled to room temperature and diluted with water (200 mL). 4-(prop-2-ynyloxy)nitrobenzene was isolated as a white solid by suction filtration (1.12 g). 1H NMR (CDCl3): δ 8.22 (d, J=9.0 Hz, 2H), 7.05 (d, J=9.0 Hz, 2H), 4.80 (d, J=2.4 Hz, 2H), 2.59 (t, J=2.4 Hz, 1H).

4-(Prop-2-ynyloxy)nitrobenzene (0.910 g, 5.13 mmol), iron (1.42 g, 25.3 mmol), and NH₄Cl (0.719 g, 12.8 mmol) were vigorously stirred in EtOH/water (1:1, 55 mL) at 70° C. for 15 minutes. The reaction mixture was filtered hot through diatomaceous earth and concentrated in vacuo. The residue was suspended in 10% 2N ammoniacal methanol in dichloromethane, sonicated, and filtered through diatomaceous earth. Concentration gave 4-(prop-2-ynyloxy)aniline as an oil which was used without further start here purification. 1H NMR (CDCl3): δ 6.82 (d, J=8.7 Hz, 2H), 6.64 (d, J=8.7 Hz, 2H), 4.61 (d, J=2.4 Hz, 2H), 2.50 (t, J=2.4 Hz, 1H).

4-(prop-2-ynyloxy)aniline (0.750 g, 5.10 mmol) and 2,4-dichloro-5-fluoropyrimidine (1.27 g, 0.760 mmol, commercially available from Sigma-Aldrich of Milwaukee, Wisconsin, USA) were stirred in MeOH/water (4:1, 35 mL) at room temperature for 18 h. The reaction mixture was diluted with EtOAc (200 mL) and washed with 1N HCl (50 mL) and brine (50 mL). The organic layer was dried (MgSO₄), filtered and concentrated in vacuo. The residue was purified by column chromatography (silica gel, hexanes ramped to EtOAc:hexanes (1:10)) to provide 2-chloro-5-fluoro-N4-[4-(prop-2-ynyloxy)phenyl]-4-pyrimidineamine as a light brown solid (0.514 g). 1H NMR (CDCl3): δ 8.03 (d, J=2.7 Hz, 1H), 7.53 (d, J=8.7 Hz, 2H), 7.02 (d, J=8.7 Hz, 2H), 6.86 (s, 1H), 4.71 (d, J=2.4 Hz, 2H), 2.55 (t, J=2.4 Hz, 1H); LCMS: purity: 99%; MS (m/e): 279 (MEt).

2-Chloro-5-fluoro-N4-[4-(prop-2-ynyloxy)phenyl]-4-pyrimidineamine (0.514 g, 1.85 mmol), 3-(aminosulfonyl)-4-methylaniline (0.689 g, 3.70 mmol, made by reduction of commercially available 2-methyl-5-nitrobenzenesulfonamide or synthesized as described below), and trifluoroacetic acid (0.186 mL, 2.41 mmol) were combined with iPrOH (6.0 mL) in a sealed vial and heated at 100° C. for 3 h. The reaction mixture was cooled to room temperature and diluted with 1N HCl (80 mL). N2-(3-Aminosulfonyl-4-methylphenyl)-5-fluoro-N4-[4-(prop-2-ynyloxy)phenyl]-2,4-pyrimidinediamine (B-I) was isolated as a white solid by suction filtration (0.703 g). 1H NMR (DMSO-d6): δ 10.08 (bs, 2H), 8.19 (d, J=4.5 Hz, 1H), 7.89 (s, 1H), 7.74 (dd, J=2.4 and 8.4 Hz, 1H), 7.58 (d, J=8.7 Hz, 2H), 7.32 (bs, 2H), 7.23 (d, J=8.4 Hz, 1H), 6.97 (d, J=8.4 Hz, 2H), 4.79 (d, J=2.1 Hz, 2H), 3.59-3.55 (m, 1H), 2.53 (s, 3H); LCMS: purity: 97%;

MS (m/e): 428 (MEt).

B-II: 5-fluoro-N2-(4-methyl-3-propionylaminosulfonylphenyl)-N4-[4-(prop-2-ynyloxy)phenyl]-2,4-pyrimidinediamine N2-(3-Aminosulfonyl-4-methylphenyl)-5-fluoro-N4-[4-(prop-2-ynyloxy)phenyl]-2,4-pyrimidinediamine, B-I, (0.200 g, 0.467 mmol), DMAP (40 mg, 0.33 mmol)) and triethylamine (0.118 mL, 0.847 mmol) were stirred in THF (6.0 mL). Propionic anhydride (0.180 mL, 1.40 mmol) was added to the solution drop wise. The reaction mixture was stirred at room temperature overnight. The solution was diluted with ethyl acetate (50 mL) and washed with water (5×25 mL) and brine (10 mL). The organic layer was dried (MgSO₄), filtered, and evaporated. The residue was suspended in ethyl acetate (25 mL), sonicated and the solid collected by filtration to give 5-fluoro-N2-(4-methyl-3-propionylaminosulfonylphenyl)-N4-[4-(prop-2-ynyloxy)phenyl]-2,4-pyrimidinediamine, B-II, (0.20 g). 1H NMR (DMSO-d6): δ 12.01 (s, 1H), 9.44 (s, 1H), 9.26 (s, 1H), 8.16 (d, J=2.4 Hz, 1H), 8.06 (dd, J=0.3 and 3.3 Hz, 1H), 8.00 (dd, J=2.1 and 7.8 Hz, 1H), 7.69 (d, J=8.7 Hz, 2H), 7.19 (d, J=8.4 Hz, 1H), 6.95 (d, J=8.7 Hz, 2H), 4.77 (d, J=2.1 Hz, 2H), 3.56 (t, J=2.1 Hz, 1H), 2.49 (s, 3H), 2.24 (q, J=7.2 Hz, 2H), 0.89 (t, J=7.2 Hz, 3H); LCMS: purity: 98%; MS (m/e): 484 (MH⁺).

B-III: 5-fluoro-N2-(4-methyl-3-propionylaminosulfonylphenyl)-N4-[4-(prop-2-ynyloxy)phenyl]-2,4-pyrimidinediamine mono-sodium salt

5-Fluoro-N2-(4-methyl-3-propionylaminosulfonylphenyl)-N4-[4-(prop-2-ynyloxy)phenyl]-2,4-pyrimidinediamine, B-II, (0.125 g, 0.258 mmol) was suspended in acetonitrile (1.5 mL) and water (1.5 mL) and cooled in an ice bath. A solution of 1N NaOH aq. (0.260 mL) was added drop wise. The reaction mixture was stirred until it became clear, filtered through glass wool, and lyophilized to give the sodium salt of B-II. ¹H NMR (DMSO-d₆): δ 9.17 (bs, 2H), 8.01 (d, J=3.6 Hz, 1H), 7.89 (s, 1H), 7.78-7.69 (m, 3H), 6.99-6.92 (m, 3H), 4.76 (d, J=2.1 Hz, 1H), 2.43 (s, 3H), 1.95 (q, J=7.2 Hz, 2H), 0.86 (t, J=7.2 Hz, 3H); LCMS: purity: 98%; MS (m/e): 484 (MH-0.

The following compounds were made in a similar fashion to those above.

B-IV: 5-Fluoro-N2-[4-methyl-3-(N-propionylaminosulfonyl)phenyl]-N4-[4-(2-propynyloxy)phenyl]-2,4-pyrimidinediamine Potassium Salt

1H NMR (DMSO-d6): δ 9.16 (s, 1H), 9.14 (s, 1H), 8.01 (d, J=3.6 Hz, 1H), 7.85 (d, J=2.1 Hz, 1H), 7.75-7.70 (m, 3H), 6.97-6.92 (m, 3H), 4.76 (d, J=1.8 Hz, 2H), 3.55 (t, J=2.4 Hz, 1H), 2.42 (s, 3H), 1.91 (q, J=7.5 Hz, 2H), 0.85 (t, J=7.5 Hz, 3H); LCMS: purity: 97%; MS (m/z): 484 (parent, MH⁺).

B-V: 5-Fluoro-N2-[4-methyl-3-(N-propionylaminosulfonyl)phenyl]-N4-[4-(2-propynyloxy)phenyl]-2,4-pyrimidinediamine Calcium Salt

1H NMR (DMSO-d6): δ 9.16 (s, 2H), 8.00 (d, J=3.6 Hz, 1H), 7.88 (d, J=1.8 Hz, 1H), 7.75-7.69 (m, 3H), 6.97-6.92 (m, 3H), 4.76 (d, J=1.8 Hz, 2H), 3.55 (t, J=2.1 Hz, 1H), 2.43 (s, 3H), 1.94 (q, J=7.5 Hz, 2H), 0.87 (t, J=7.5 Hz, 3H); LCMS: purity: 98%; MS (m/z): 484 (parent, MH⁺).

B-VI: 5-Fluoro-N2-[4-methyl-3-(N-propionylaminosulfonyl)phenyl]-N4-[4-(2-propynyloxy)phenyl]-2,4-pyrimidinediamine Arginine Salt

1H NMR (D₂O): 8 7.61 (d, J=3.9 Hz, 1H), 7.57-7.55 (m, 1H), 7.36-7.31 (m, 1H), 7.12 (d, J=8.7 Hz, 2H), 6.88 (d, J=8.7 Hz, 1H), 6.72 (d, J=9.0 Hz, 2H), 4.77-4.75 (m, 2H), 3.60 (t, J=6.0 Hz, 1H), 3.09 (t, J=6.9 Hz, 2H), 2.84-2.81 (m, 1H), 2.35 (s, 3H), 2.03 (q, J=5.7 Hz, 2H), 1.80-1.72 (m, 2H), 1.61-1.48 (m, 2H), 0.855 (t, J=7.5 Hz, 3H); LCMS: purity: 98%; MS (m/z): 484 (parent, MH⁺).

B-VII: 5-Fluoro-N2-[4-methyl-3-(N-propionylaminosulfonyl)phenyl]-N4-[4-(2-propynyloxy)phenyl]-2,4-pyrimidinediamine Choline Salt

1H NMR (DMSO-d6): δ 9.16 (s, 2H), 8.00 (d, J=3.6 Hz, 1H), 7.85 (d, J=1.8 Hz, 1H), 7.75-7.69 (m, 3H), 6.97-6.90 (m, 3H), 5.27 (t, J=4.8 Hz, 1H), 4.76 (d, J=1.8 Hz, 2H), 3.86-3.77 (m, 2H), 3.56-3.54 (m, 1H), 3.40-3.54 (m, 2H), 3.08 (s, 9H), 2.42 (s, 3H); LCMS: purity: 99%; MS (m/z): 484 (parent, MH⁺).

### Synthesis of 5-amino-2-methylbenzenesulfonamide

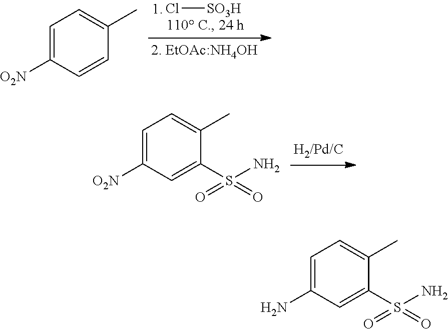

4-methylnitrobenzene (20 mmol) is treated at 0° C. with chlorosulfonic acid (5.29 mL, 80 mmol) and then, after bringing the homogeneous solution to room temperature, it was stirred at 110° C. for 24 hours. The resulting slurry was then poured over ice water (100 gm), extracted with diethyl ether (3×75 mL), and the organic phase washed with water (75 mL), then dried over anhydrous sodium sulfate. The solvent was then removed under reduced pressure to afford the crude sulfonyl chloride which was taken up in ethyl acetate and stirred with ammonium hydroxide overnight at room temperature. After the ethyl acetate layer was separated, the aqueous layer was extracted with ethyl acetate. The organic layers were combined, dried over anhydrous sodium sulfate and the solvent was removed under reduced pressure. The oil obtained was purified by column chromatography (silica gel, hexanes then 10%, 20%, up to 50% ethyl acetate in hexanes to afford 3-aminosulfonyl-4-methylnitrobenzene, LCMS: purity: 95%; MS (m/e): 217 (MI-1⁺).

To a solution of 3-aminosulfonyl-4-methylnitrobenzene in dichloromethane and methanol was added 10% Pd/C and the mixture shaken under a hydrogen atmosphere at 50 psi for 15 minutes. The mixture was filtered through diatomaceous earth and the filter cake was washed with methanol. The combined organic solvents were concentrated under reduced pressure to give crude product, which was further purified by flash column chromatography (ethyl acetate: hexanes 1:1) to give 5-amino-2-methylbenzenesulfonamide, LCMS: purity: 87%; MS (m/e): 187 (MI-1⁺).

C. Synthesis of pyrazole compounds

Disclosed pyrazole compounds can be prepared as exemplified below, and as will be understood by a person of ordinary skill in the art in organic synthesis. An exemplary synthesis may include the following 1^{st} reaction step according to Scheme VIII:

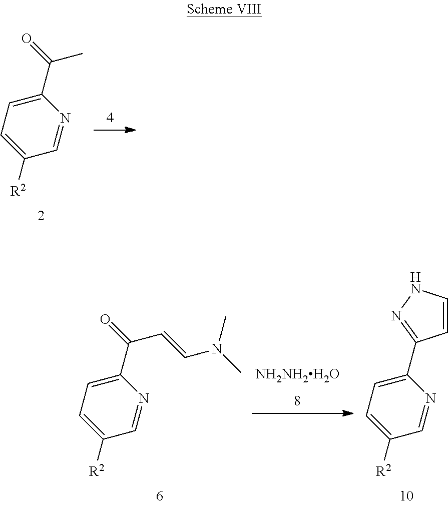

Acetyl compound 2 is reacted with dimethylformamide dimethylacetal 4 to form intermediate compound 6, at a temperature suitable to facilitate a reaction. A suitable temperature is typically from 85° C. to 130° C. Intermediate compound 6 is then reacted with hydrazine hydrate 8 to form the pyrazole compound 10. The reaction is performed in a suitable solvent, for example, an alcohol such as ethanol, methanol or isopropanol, and is typically heated, such as to reflux.

A 2^{nd} reaction step in the exemplary synthesis is provided below according to Scheme IX:

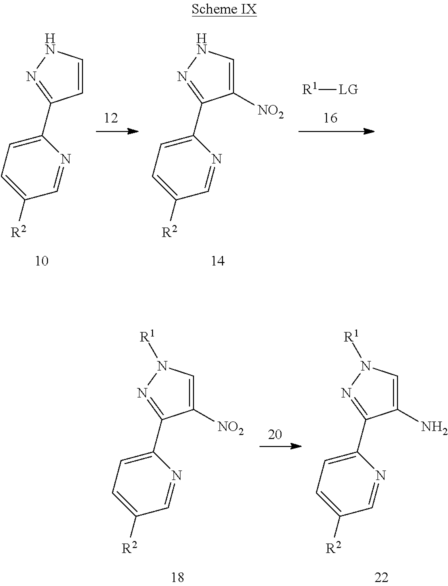

Compound 10 is nitrated using a suitable nitrating reagent or mixture of reagents 12 to form compound 14. Suitable nitrating conditions include reacting compound 10 with nitric acid, such as fuming nitric acid, optionally in the presence of sulfuric acid. Typically, compound 10 and the nitric acid are added slowly, one to the other. Cooling, such as by an ice bath, may be used to maintain the reaction temperature within a suitable range, such as from about 0° C. to less than 50° C., from 0° C. to 20° C., or from 0° C. to 10° C. After the addition is complete the reaction is allowed to proceed until the reaction is substantially complete, and may be allowed to warm to room temperature to facilitate the reaction. Optionally, additional nitrating reagent, or mixture of nitrating reagents, may be added to facilitate the reaction proceeding to completion.

The reaction is then quenched, such as by addition to water and/or ice, and the product is separated or extracted from the aqueous and purified if required. Purification techniques suitable for purifying a product from any reaction disclosed herein include, but are not limited to, crystallization, distillation and/or chromatography. With continued reference to Scheme IX, compound 14 is then reacted with compound 16 to form compound 18. Compound 16 comprises a desired R^{I} moiety and a suitable leaving group, LG. Suitable leaving groups include any group that will act as a leaving group to facilitate the addition of the R^{I} moiety to compound 14. Suitable leaving groups include, but are not limited to, halogens, typically bromo, chloro or iodo, and tosylate or mesylate groups. Compound 14 is reacted with compound 16 in a suitable solvent and typically in the presence of a base. Suitable solvents include any solvent that facilitates the reaction, such as aprotic solvents. Suitable solvents include, but are not limited to, DMF, THF, DMSO, acetonitrile, chlorinated solvents such as dichloromethane and chloroform, DMA, dioxane, N-methyl pyrrolidone, or combinations thereof. Suitable bases include any base that will facilitate the reactions, such as a hydride, typically sodium hydride, or a carbonate, such as potassium carbonate, sodium carbonate, or cesium carbonate. The reaction may be heated, such as to 50° C., 100° C. or higher, as required, or the reaction may proceed at room temperature. Compound 18 is then isolated from the reaction mixture and purified if required.

Compound 18 is then reacted with a reducing agent 20 suitable to reduce the nitro moiety to an amine. Suitable reducing agents include, but are not limited to: hydrogen gas in the presence of a catalyst, such as a palladium catalyst; a borohydride, such as sodium borohydride, optionally in the presence of a catalyst, such as a nickel catalyst; zinc metal in acetic acid; or iron powder in water or water and acid. In certain embodiments, hydrogen gas is used, in the presence of a palladium on carbon catalyst, and in a suitable solvent, such as ethyl acetate or methanol. In some embodiments, a combination of reducing agents and/or techniques are used. For example, reduction may be initially performed using a first method comprising a first reducing agent and/or technique, but result in a mixture of products. The first method may be repeated, and/or a second method may be performed, comprising a second reducing agent and/or technique. Once the reaction is complete, as indicated by an analytical technique such as LC-MS, TLC or

HPLC, the product compound 22 is isolated and purified if necessary.

A 3r^{d} step of the exemplary reaction sequence is provided below according to Scheme X:

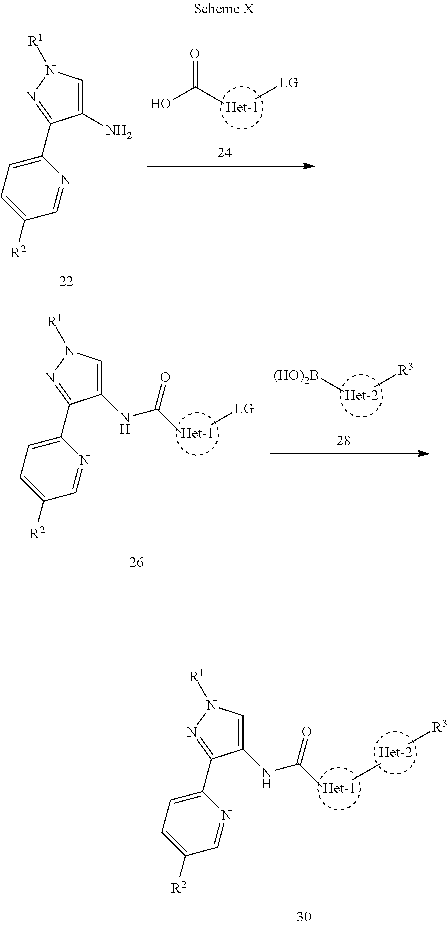

Compound 22 is reacted with a carboxylic acid 24 to form compound 26. The carboxylic acid 24 is activated by any suitable method and then reacted with the amine on compound 22. Suitable activation methods include, but are not limited to: forming the acid chloride by treatment with thionyl chloride; by treatment with 1-[Bis(dimethylamino)methylene]-1H-1,2,3-triazolo[4,5-b]pyridinium 3-oxid hexafluorophosphate (HATU) and a base such as diisopropylethylamine (DIPEA); by treatment with carbonyldiimidazole (CDI); or by treatment with a carbodiimide, such as dicyclohexylcarbodiimide (DCC) or 1-Ethyl-3-(3-dimethylaminopropyl)carbodiimide (EDC).

Compound 26 is then coupled with compound 28 to form compound 30 using any coupling reaction suitable to form a bond between two rings. In the example above, a boronic acid coupling is shown, where the leaving group LG on compound 26 is typically bromo or iodo. Other suitable coupling functional groups include trialkyl tin or boronic esters. The coupling reaction typically proceeds in the presence of a suitable catalyst. For a boronic acid coupling, the catalyst typically is a palladium catalyst, such as PdCl₂(dpp02, Pd[P(Ph)_{3]2}Cl₂, palladium acetate and triphenyl phosphine, or tetrakis(triphenylphosphine)palladium(O). The reaction is performed in the presence of a base, such as sodium, potassium or cesium carbonate, and is performed in a suitable solvent or solvent mixture, such as dioxane, dioxane/water or DME/ethanol/water. The reaction may be heated at a suitable temperature, such as from 50° C. to 125° C., typically about 100° C., and/or agitated for a suitable period of time, such as from 1 hour to 3 days, from 6 hours to 24 hours, or from 12 hours to 18 hours, to facilitate the reaction proceeding to completion. Compound 30 is then isolated from the reaction mixture and purified by a suitable technique.

An alternative exemplary synthesis may include the following 1^{st} reaction step according to Scheme XI:

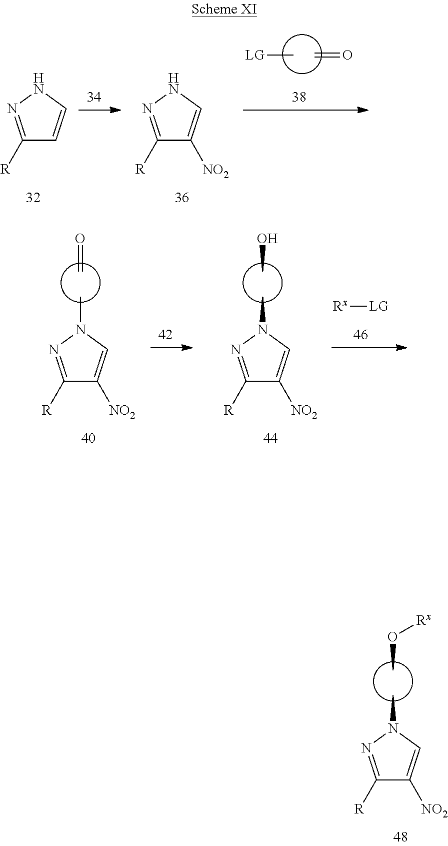

Compound 32 is nitrated using a suitable nitrating reagent or mixture of reagents 34 to form compound 36. Suitable nitrating conditions include reacting compound 32 with nitric acid, such as fuming nitric acid, optionally in the presence of sulfuric acid. Typically, compound 32 and the nitric acid are added slowly, one to the other. Cooling, such as by an ice bath, may be used to maintain the reaction temperature within a suitable range, such as from about 0° C. to less than 50° C., from 0° C. to 20° C., or from 0° C. to 10° C. After the addition is complete the reaction is allowed to proceed until the reaction is substantially complete, and may be allowed to warm to room temperature to facilitate the reaction. Optionally, additional nitrating reagent, or mixture of nitrating reagents, may be added to facilitate the reaction proceeding to completion.

The reaction is then quenched, such as by addition to water and/or ice, and the product is separated or extracted from the aqueous and purified if required. Purification techniques suitable for purifying a product from any reaction disclosed herein include, but are not limited to, crystallization, distillation and/or chromatography. With continued reference to Scheme XI, compound 36 is then reacted with compound 38 to form compound 40. Compound 38 comprises a desired ring, such as a cyclobutyl, cyclopentyl, or cyclohexyl ring, and a suitable leaving group, LG. Suitable leaving groups include any group that will act as a leaving group to facilitate the addition of the ring to compound 36. Suitable leaving groups include, but are not limited to, halogens, typically bromo, chloro or iodo, and tosylate or mesylate groups. Compound 36 is reacted with compound 38 in a suitable solvent and typically in the presence of a base. Suitable solvents include any solvent that facilitates the reaction, such as aprotic solvents. Suitable solvents include, but are not limited to, DMF, THF, DMSO, acetonitrile, chlorinated solvents such as dichloromethane and chloroform, DMA, dioxane, N-methyl pyrrolidone, or combinations thereof. Suitable bases include any base that will facilitate the reactions, such as a hydride, typically sodium hydride, or a carbonate, such as potassium carbonate, sodium carbonate, or cesium carbonate. The reaction may be heated, such as to 50° C., 100° C. or higher, as required, or the reaction may proceed at room temperature. Compound 40 is then isolated from the reaction mixture and purified if required.

Compound 40 is then reacted with a reducing agent 42 suitable to reduce the carbonyl moiety to a hydroxyl. Suitable reducing agents include, but are not limited to, sodium borohydride, di-isobutyl aluminum hydride, or lithium aluminum hydride. The reaction is performed in a solvent suitable to facilitate the reaction, such as an alcohol, particularly methanol or ethanol; THF; or diethyl ether. The reaction may be heated, such as to 50° C., 100° C. or higher, as required, cooled, such as to below 20° C., below 10° C., below 0° C. or lower, or the reaction may proceed at room temperature. Once the reaction is complete, as indicated by an analytical technique such as LC-MS, TLC or HPLC, the product compound 44 is isolated and purified if necessary, by a suitable technique, such as column chromatography.

Optionally, compound 44 may be reacted with compound 46 to form compound 48. Compound 46 comprises a desired RX moiety and a suitable leaving group, LG. Suitable leaving groups include any group that will act as a leaving group to facilitate the addition of the RX moiety to compound 44. Suitable leaving groups include, but are not limited to, halogens, typically bromo, chloro or iodo, and tosylate or mesylate groups. Compound 44 is reacted with compound 46 in a suitable solvent and typically in the presence of a base or other reagent or reagents that facilitate the reaction. Suitable solvents include any solvent that facilitates the reaction, such as aprotic solvents. Suitable solvents include, but are not limited to, DMF, THF, DMSO, acetonitrile, chlorinated solvents such as dichloromethane and chloroform, DMA, dioxane, N-methyl pyrrolidone, or combinations thereof. Suitable bases or reagents that facilitate the reaction include, but are not limited to, silver triflate, 2,6-di-t-butylpyridine, sodium hydride, or combinations thereof. Typically, compound 46 is slowly combined with the reaction. Cooling, such as by an ice bath, may be used to maintain the reaction temperature within a suitable range, such as from about 0° C. to less than 50° C., from 0° C. to 20° C., or from 0° C. to 10° C. After the addition is complete the reaction is allowed to proceed until the reaction is substantially complete, and may be allowed to warm to room temperature, or the reaction may be heated, such as to 50° C., 100° C. or higher, to facilitate the reaction. Once the reaction is complete, as indicated by an analytical technique such as LC-MS, TLC or HPLC, the product compound 48 is isolated and purified if necessary, by a suitable technique, such as column chromatography. Alternatively, compound 40 may be prepared by an exemplary synthetic route according to Scheme

XII:

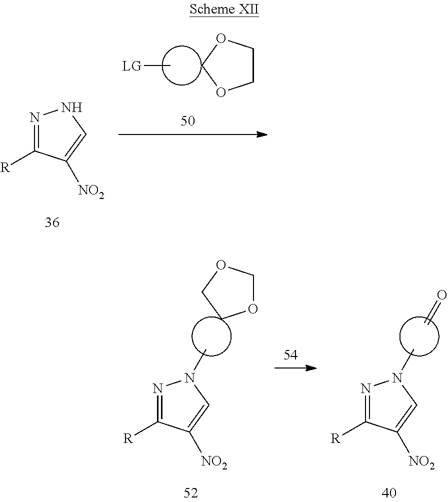

With respect to Scheme XII, compound 36 is reacted with compound 50 to form compound 52. Compound 50 comprises a desired ring, such as a cyclobutyl, cyclopentyl, or cyclohexyl ring, a suitable leaving group,

LG, and a protected carbonyl moiety, such as an acetal or a ketal. In the example above a cyclic ketal moiety is shown. Suitable leaving groups include any group that will act as a leaving group to facilitate the addition of the ring to compound 36, and include, but are not limited to, halogens, typically bromo, chloro or iodo, and tosylate or mesylate groups. Compound 36 is reacted with compound 50 in a suitable solvent and typically in the presence of a base. Suitable solvents include any solvent that facilitates the reaction, such as aprotic solvents. Suitable solvents include, but are not limited to, DMF, THF, DMSO, acetonitrile, chlorinated solvents such as dichloromethane and chloroform, DMA, dioxane, N-methyl pyrrolidone, or combinations thereof. Suitable bases include any base that will facilitate the reactions, such as a hydride, typically sodium hydride, or a carbonate, such as potassium carbonate, sodium carbonate, or cesium carbonate. The reaction may be heated, such as to 50° C., 100° C. or higher, as required, or the reaction may proceed at room temperature. Compound 52 is then isolated from the reaction mixture and purified if required by a suitable technique, such as column chromatography.

Compound 52 is then reacted with a suitable reagent 54 to form compound 40. Reagent 54 may be any reagent suitable to remove the protecting group and/or form the carbonyl moiety. In the exemplary synthesis shown in Scheme 5, the protecting group is a cyclic ketal, and suitable reagents 54 include, but are not limited to, pyridinium tosylate (PPTS), para-toluene sulfonic acid, hydrochloric acid, or acetic acid. The reaction is performed in a solvent or mixture of solvents suitable to facilitate the reaction, such as acetone,

THF, acetic acid, water, or a combination thereof The reaction may be heated, such as to 50° C., 100° C. or higher, or at reflux, as required, or the reaction may proceed at room temperature. Compound 40 is then isolated from the reaction mixture and purified if required by a suitable technique, such as column chromatography.

A 2^{nd} step of the exemplary reaction sequence is provided below according to Scheme XIII:

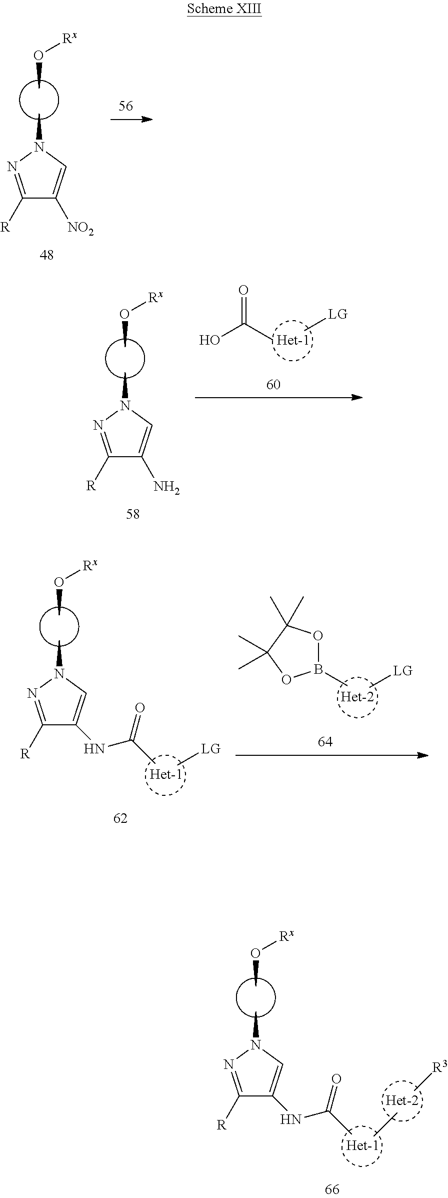

Compound 48 is then reacted with a reducing agent 56 suitable to reduce the nitro moiety to an amine. In certain embodiments where the desired product compound comprises a hydroxyl moiety, compound 44 may be used in place of compound 48. Suitable reducing agents include, but are not limited to: hydrogen gas in the presence of a catalyst, such as a palladium catalyst; a borohydride, such as sodium borohydride, optionally in the presence of a catalyst, such as a nickel catalyst; zinc metal in acetic acid; or iron powder in water or water and acid. In certain embodiments, hydrogen gas is used, in the presence of a palladium on carbon catalyst, and in a suitable solvent, such as ethyl acetate or methanol. In some embodiments, a combination of reducing agents and/or techniques are used. For example, reduction may be initially performed using a first method comprising a first reducing agent and/or technique, but result in a mixture of products. The first method may be repeated, and/or a second method may be performed, comprising a second reducing agent and/or technique. Once the reaction is complete, as indicated by an analytical technique such as LC-MS, TLC or HPLC, the product compound 58 is isolated and purified if necessary.

Compound 58 is reacted with a carboxylic acid 60 to form compound 62. The carboxylic acid 60 is activated by any suitable method and then reacted with the amine on compound 58. Suitable activation methods include, but are not limited to: forming the acid chloride by treatment with thionyl chloride; by treatment with 1-[Bis(dimethylamino)methylene]-1H-1,2,3-triazolo[4,5-b]pyridinium 3-oxid hexafluorophosphate (HATU) and a base such as diisopropylethylamine (DIPEA); by treatment with carbonyldiimidazole (CDI); or by treatment with a carbodiimide, such as dicyclohexylcarbodiimide (DCC) or 1-Ethyl-3-(3-dimethylaminopropyl)carbodiimide (EDC).

Compound 62 is then coupled with compound 64 to form compound 66 using any coupling reaction suitable to form a bond between two rings. In the example above, a boronic ester coupling is shown, where the leaving group LG on compound 62 is typically bromo or iodo. Other suitable coupling functional groups include trialkyl tin or boronic acids. The coupling reaction typically proceeds in the presence of a suitable catalyst. For a boronic ester or boronic acid coupling, the catalyst typically is a palladium catalyst, such as PdCl₂(dppf)₂, Pd[P(Ph)₃]₂Cl₂, palladium acetate and triphenyl phosphine, or tetrakis(triphenylphosphine)palladium(0). The reaction is performed in the presence of a base, such as sodium, potassium or cesium carbonate, and is performed in a suitable solvent or solvent mixture, such as dioxane, dioxane/water or DME/ethanol/water. The reaction may be heated at a suitable temperature, such as from 50° C. to 125° C., typically about 100° C., and/or agitated for a suitable period of time, such as from 1 hour to 3 days, from 6 hours to 24 hours, or from 12 hours to 18 hours, to facilitate the reaction proceeding to completion. Compound 66 is then isolated from the reaction mixture and purified by a suitable technique.

Certain embodiments may comprise a phosphate moiety. Scheme XIV provides an exemplary synthesis of certain such embodiments:

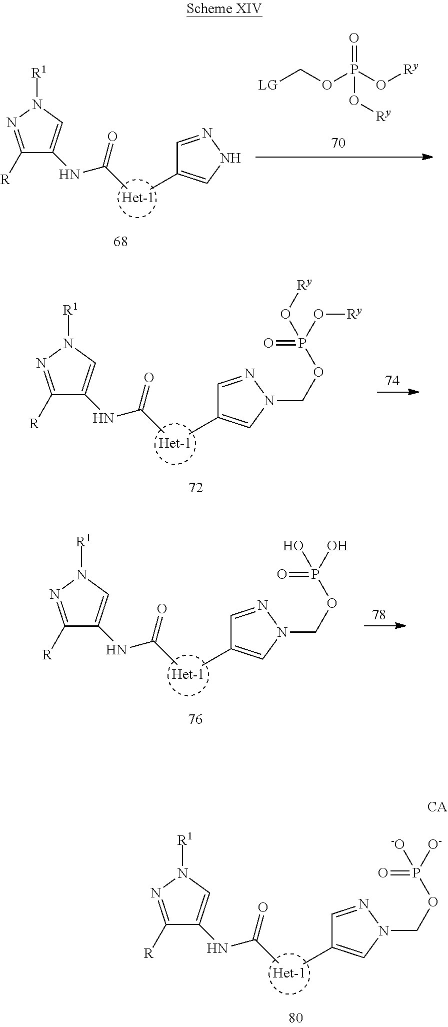

Compound 68 is reacted with compound 70 to form compound 72. Compound 70 comprises desired R^{y} moieties and a suitable leaving group, LG. Typical R^{y} moieties include, but are not limited to aliphatic, such as alkyl, typically methyl, ethyl, propyl, isopropyl or t-butyl; aryl; heteroaliphatic; or heterocyclic. The two RY moieties may be the same or different. Suitable leaving groups include, but are not limited to, halogens, typically bromo, chloro or iodo, and tosylate or mesylate groups. Compound 68 is reacted with compound 70 in a suitable solvent and typically in the presence of a base. Suitable solvents include any solvent that facilitates the reaction, such as aprotic solvents. Suitable solvents include, but are not limited to, DMF, THF, DMSO, acetonitrile, chlorinated solvents such as dichloromethane and chloroform, DMA, dioxane, N-methyl pyrrolidone, or combinations thereof. Suitable bases include any base that will facilitate the reactions, such as a hydride, typically sodium hydride, or a carbonate, such as potassium carbonate, sodium carbonate, or cesium carbonate. The reaction may be heated, such as to 50° C., 100° C. or higher, as required, or the reaction may proceed at room temperature. Compound 72 is then isolated from the reaction mixture and purified if required.

Compound 72 is then reacted with compound 74 to form compound 76. Compound 74 may be any compound suitable to form the acid moieties in compound 76. Compound 74 may be an acidic reagent, such as trifluoroacetic acid, hydrochloride acid, or hydrobromic acid, or it may be a basic reagent, such as sodium hydroxide, lithium hydroxide or potassium hydroxide. Suitable solvents include, but are not limited to, chlorinated solvents such as dichloromethane and chloroform, alcohols such as methanol and ethanol, water, or combinations thereof The reaction may be heated, such as to 50° C., 100° C. or higher, as required, cooled, such as to below 20° C., below 10° C., below 0° C. or lower, or the reaction may proceed at room temperature. Once the reaction is complete, as indicated by an analytical technique such as LC-MS, TLC or HPLC, the product compound 76 is isolated and purified if necessary, by a suitable technique, such as by agitating, such as by stirring or sonication, in a suitable solvent or solvent system. Suitable solvents or solvent systems include, but are not limited to, acetone/water, acetone, diethyl ether, or alcohol/water. Compound 76 is then reacted with compound 78 to form the salt compound 80. Compound 78 can be any compound that will provide a suitable counterion CA for the salt compound 80, such as calcium hydroxide, sodium hydroxide, potassium hydroxide, lithium hydroxide, ammonia, trimethylamine, tris(hydroxymethyl)aminomethane, or an amino acid such as lysine or arginine. A person of ordinary skill in the art will appreciate that if counter ion CA has a single positive charge, as in Na⁺, K⁺, Li⁺, or NH₄⁺, then compound 80 will comprise two CA ions, whereas if counter ion CA has two positive charges, as in CA′ compound 80 will comprise one CA ion.

IV. Compositions comprising a compound disclosed herein

The disclosed compounds may be used alone or in combination, and/or in combination with, or adjunctive to, at least one second therapeutic agent, and further the compound(s), and the at least one second therapeutic if present, may be used in combination with any suitable additive useful for forming compositions for administration to a subject. Additives can be included in pharmaceutical compositions for a variety of purposes, such as to dilute a composition for delivery to a subject, to facilitate processing of the formulation, to provide advantageous material properties to the formulation, to facilitate dispersion from a delivery device, to stabilize the formulation (e.g., antioxidants or buffers), to provide a pleasant or palatable taste or consistency to the formulation, or the like. Typical additives include, by way of example and without limitation: pharmaceutically acceptable excipient, including carriers and/or adjuvants, such as mono-, di-, and polysaccharides, sugar alcohols and other polyols, such as, lactose, glucose, raffinose, melezitose, lactitol, maltitol, trehalose, sucrose, mannitol, starch, or combinations thereof; surfactants, such as sorbitols, diphosphatidyl choline, and lecithin; bulking agents; buffers, such as phosphate and citrate buffers; anti-adherents, such as magnesium stearate; binders, such as saccharides (including disaccharides, such as sucrose and lactose,), polysaccharides (such as starches, cellulose, microcrystalline cellulose, cellulose ethers (such as hydroxypropyl cellulose), gelatin, synthetic polymers (such as polyvinylpyrrolidone, polyalkylene gylcols); coatings (such as cellulose ethers, including hydroxypropylmethyl cellulose, shellac, corn protein zein, and gelatin); release aids (such as enteric coatings); disintegrants (such as crospovidone, crosslinked sodium carboxymethyl cellulose, and sodium starch glycolate); fillers (such as dibasic calcium phosphate, vegetable fats and oils, lactose, sucrose, glucose, mannitol, sorbitol, calcium carbonate, and magnesium stearate); flavors and sweeteners (such as mint, cherry, anise, peach, apricot or licorice, raspberry, and vanilla; lubricants (such as minerals, exemplified by talc or silica, fats, exemplified by vegetable stearin, magnesium stearate or stearic acid); preservatives (such as antioxidants exemplified by vitamin A, vitamin E, vitamin C, retinyl palmitate, and selenium, amino acids, exemplified by cysteine and methionine, citric acid and sodium citrate, parabens, exemplified by methyl paraben and propyl paraben); colorants; compression aids; emulsifying agents; encapsulation agents; gums; granulation agents; and combinations thereof.

### V. Combinations of Therapeutic Agents

The disclosed compounds may be used alone, in combination with another disclosed compound, and/or as an adjunct to, or in combination with, other established therapies. In another aspect, the compounds may be used in combination with other therapeutic agents useful for treating CRS, and/or other diseases or conditions. The compounds and/or other agents may be administered simultaneously, sequentially in any order, by the same route of administration, or by a different route. In some embodiments, a second therapeutic agent is an analgesic, an antibiotic, an anticoagulant, an antibody, an anti-inflammatory agent, an immunosuppressant, a guanylate cyclase-C agonist, an intestinal secretagogue, an antiviral, anticancer, antifungal, or a combination thereof. In certain embodiments, the second therapeutic is an anti-inflammatory agent, an immunosuppressant and/or may be a steroid. In certain conditions, such as a COVID-19 infection, a patient is also treated with an antiviral agent, such as remdesivir or GS-441524, in combination with the present compounds.

The anti-inflammatory agent may be a steroid, such as budesonide, dexamethasone, prednisone or the like, or a nonsteroidal anti-inflammatory agent. In certain embodiments, the nonsteroidal anti-inflammatory agent is selected from aminosalicylates (e.g., sulfasalazine, mesalamine, olsalazine, and balsalazide), cyclooxygenase inhibitors (COX-2 inhibitors, such as rofecoxib, celecoxib), diclofenac, etodolac, famotidine, fenoprofen, flurbiprofen, ketoprofen, ketorolac, ibuprofen, indomethacin, meclofenamate, mefenamic acid, meloxicam, nambumetone, naproxen, oxaprozin, piroxicam, salsalate, sulindac, tolmetin, or a combination thereof. DONE

In some embodiments, the immunosuppressant is mercaptopurine; a corticosteroid, such as dexamethasone, hydrocortisone, prednisone, methylprednisolone and prednisolone; an alkylating agent, such as cyclophosphamide; a calcineurin inhibitor, such as cyclosporine, sirolimus and tacrolimus; an inhibitor of inosine monophosphate dehydrogenase (IMPDH) such as mycophenolate, mycophenolate mofetil and azathioprine; and agents designed to suppress cellular immunity while leaving the recipient's humoral immunologic response intact, including various antibodies (for example, antilymphocyte globulin (ALG), antithymocyte globulin (ATG), monoclonal anti-T-cell antibodies (OKT3)) and irradiation; or a combination thereof. In one embodiment, the antibody is infliximab. Azathioprine is currently available from Salix Pharmaceuticals, Inc. under the brand name Azasan; mercaptopurine is currently available from Gate Pharmaceuticals, Inc. under the brand name Purinethol; prednisone and prednisolone are currently available from Roxane Laboratories, Inc.; Methyl prednisolone is currently available from Pfizer; sirolimus (rapamycin) is currently available from Wyeth-Ayerst under the brand name Rapamune; tacrolimus is currently available from Fujisawa under the brand name Prograf; cyclosporine is current available from Novartis under the brand name Sandimmune and Abbott under the brand name Gengraf; IMPDH inhibitors such as mycophenolate mofetil and mycophenolic acid are currently available from Roche under the brand name Cellcept and Novartis under the brand name Myfortic; azathioprine is currently available from Glaxo Smith Kline under the brand name Imuran; and antibodies are currently available from Ortho Biotech under the brand name Orthoclone, Novartis under the brand name Simulect (basiliximab) and Roche under the brand name Zenapax (daclizumab).

In certain embodiments, the second therapeutic is, or comprises, a steroid, such as a corticosteroid, including, but not limited to, glucocorticoids and/or mineralocorticoids. Steroids suitable for use in combination with the disclosed compounds include synthetic and non-synthetic glucocorticoids. Exemplary steroids, such as glucocorticoids, suitable for use in the disclosed methods include, but are not limited to, alclomethasones, algestones, beclomethasones (e.g. beclomethasone dipropionate), betamethasones (e.g. betamethasone 17-valerate, betamethasone sodium acetate, betamethasone sodium phosphate, betamethasone valerate), budesonides, clobetasols (e.g. clobetasol propionate), clobetasones, clocortolones (e.g. clocortolone pivalate), cloprednols, corticosterones, cortisones, cortivazols, deflazacorts, desonides, desoximethasones, dexamethasones (e.g. dexamethasone 21-phosphate, dexamethasone acetate, dexamethasone sodium phosphate), diflorasones (e.g. diflorasone diacetate), diflucortolones, difluprednates, enoxolones, fluazacorts, flucloronides, fludrocortisones (e.g., fludrocortisone acetate), flumethasones (e.g. flumethasone pivalate), flunisolides, fluocinolones (e.g. fluocinolone acetonide), fluocinonides, fluocortins, fluocortolones, fluorometholones (e.g. fluorometholone acetate), fluperolones (e.g., fluperolone acetate), fluprednidenes, fluprednisolones, flurandrenolides, fluticasones (e.g. fluticasone propionate), formocortals, halcinonides, halobetasols, halometasones, halopredones, hydrocortamates, hydrocortisones (e.g. hydrocortisone 21-butyrate, hydrocortisone aceponate, hydrocortisone acetate, hydrocortisone buteprate, hydrocortisone butyrate, hydrocortisone cypionate, hydrocortisone hemisuccinate, hydrocortisone probutate, hydrocortisone sodium phosphate, hydrocortisone sodium succinate, hydrocortisone valerate), loteprednol etabonate, mazipredones, medrysones, meprednisones, methylprednisolones (methylprednisolone aceponate, methylprednisolone acetate, methylprednisolone hemi succinate, methylprednisolone sodium succinate), mometasones (e.g., mometasone furoate), paramethasones (e.g., paramethasone acetate), prednicarbates, prednisolones (e.g. prednisolone 25-diethylaminoacetate, prednisolone sodium phosphate, prednisolone 21-hemi succinate, prednisolone acetate; prednisolone farnesylate, prednisolone hemisuccinate, prednisolone-21 (beta-D-glucuronide), prednisolone metasulphobenzoate, prednisolone steaglate, prednisolone tebutate, prednisolone tetrahydrophthalate), prednisones, prednivals, prednylidenes, rimexolones, tixocortols, triamcinolones (e.g. triamcinolone acetonide, triamcinolone benetonide, triamcinolone hexacetonide, triamcinolone acetonide 21-palmitate, triamcinolone diacetate), or any combination thereof. Additional information concerning steroids, and the salts thereof, can be found, for example, in Remington's Pharmaceutical Sciences, A. Osol, ed., Mack Pub. Co., Easton, Pa. (16th ed. 1980).

In some examples, the steroid is a glucocorticoid, and may be selected from cortisone, dexamethasone, hydrocortisone, methylprednisolone, prednisolone, prednisone, or a combination thereof. In a particular example, the steroid is, or comprises, prednisone. In another particular example, the steroid is, or comprises, dexamethasone.

### VI. Formulations and Administration

Pharmaceutical compositions comprising one or more of the disclosed compounds (including salts, solvates, N-oxides and/or prodrugs thereof) may be manufactured by means of conventional mixing, dissolving, granulating, dragee-making, levigating, emulsifying, encapsulating, entrapping or lyophilization processes. The compositions may be formulated in conventional manner using one or more physiologically acceptable excipients, diluents, carriers, adjuvants or auxiliaries to provide preparations which can be used pharmaceutically. A wide variety of suitable pharmaceutical compositions are known in the art. See, e.g., Remington: The Science and Practice of Pharmacy, volume I and volume II (22n^{d} Ed., University of the Sciences, Philadelphia).

The disclosed compound(s), or a prodrug thereof, may be formulated in the pharmaceutical compositions per se, or in the form of a solvate, N-oxide or pharmaceutically acceptable salt. Typically, such salts are more soluble in aqueous solutions than the corresponding free acids and bases, but salts having lower solubility than the corresponding free acids and bases may also be formed.

Pharmaceutical compositions comprising one or more of the disclosed compounds may take a form suitable for virtually any mode of administration, including, for example, topical, ocular, oral, buccal, systemic, nasal, injection, such as i.v. or i.p., transdermal, rectal, vaginal, sublingual, urethral (e.g., urethral suppository) etc., or a form suitable for administration by inhalation or insufflation. In certain embodiments, the mode of administration is oral or injection.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives.

Alternatively, the injectable formulation may be provided in powder form for reconstitution with a suitable vehicle, including but not limited to sterile, pyrogen-free water, buffer, dextrose solution, etc., before use. To this end, the disclosed compound(s) maybe dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients, such as: binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); and/or wetting agents (e.g., sodium lauryl sulfate). The tablets may be coated by methods well known in the art with, for example, sugars, films or enteric coatings.

Additionally, the pharmaceutical compositions containing the disclosed compound(s) as an active ingredient or solvates, N-oxides, pharmaceutically acceptable salts or prodrug(s) thereof in a form suitable for oral use, may also include, for example, troches, lozenges, aqueous or oily suspensions, dispersible powders or granules, emulsions, hard or soft capsules, or syrups or elixirs. Compositions intended for oral use can be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient (including a prodrug) in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch, or alginic acid); binding agents (e.g. starch, gelatin or acacia); and lubricating agents (e.g. magnesium stearate, stearic acid or talc). The tablets can be uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques described in the U.S. Pat. Nos. 4,256,108; 4,166,452; and 4,265,874 to form osmotic therapeutic tablets for control release. The pharmaceutical compositions of the invention may also be in the form of oil-in-water emulsions. Tablets may also be film coated, and the file coating can comprise one or more of polyvinyl alcohol, titanium dioxide, polyethylene glycol 3350, talc, iron oxide yellow, and iron oxide red.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as: suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the disclosed compound as is well known.

For buccal administration, the compositions may take the form of tablets or lozenges formulated in conventional manner.

For topical administration, the disclosed compound(s) (including solvates, N-oxides or pharmaceutically acceptable salt and/or prodrug(s) thereof) may be formulated as solutions, gels, ointments, creams, suspensions, etc. as are well-known in the art.

For rectal and vaginal routes of administration, the active compound(s) may be formulated as solutions (for retention enemas) suppositories or ointments containing conventional suppository bases, such as cocoa butter or other glycerides.

For nasal administration or administration by inhalation or insufflation, the disclosed compound(s), solvates, N-oxides, pharmaceutically acceptable salts or prodrug(s), can be conveniently delivered in the form of an aerosol spray from pressurized packs or a nebulizer with the use of a suitable propellant, e.g.,) dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

The pharmaceutical compositions can be in the form of a sterile injectable aqueous or oleagenous suspension. This suspension can be formulated according to the known art using those suitable dispersing or wetting agents and suspending agents which have been mentioned above. The sterile injectable preparation may also be a sterile injectable solution or suspension in a non-toxic parenterally-acceptable diluent or solvent. Among the acceptable vehicles and solvents that can be employed are water, Ringer's solution and isotonic sodium chloride solution.

According to the present invention, a form of the disclosed compound(s), solvates, N-oxides, pharmaceutically acceptable salts or prodrug(s) thereof, can also be delivered by any of a variety of inhalation devices and methods known in the art, including, for example: U.S. Pat. Nos. 6,241,969; 6,060,069; 6,238,647; 6,335,316; 5,364,838; 5,672,581; WO96/32149; WO95/24183; U.S. Pat. No. 5,654,007; 5,404,871; 5,672,581; 5,743,250; 5,419,315; 5,558,085; WO98/33480; U.S. Pat. Nos. 5,364,833; 5,320,094; 5,780,014; 5,658,878; 5,518,998; 5,506,203; 5,661,130; 5,655,523; 5,645,051; 5,622,166; 5,577,497; 5,492,112; 5,327,883; 5,277,195; U.S. Publication No. 20010041190; U.S. Publication No. 20020006901; and U.S. Publication No. 20020034477.

Included among the devices which can be used to administer a form of the active compound(s) are those well-known in the art, such as, metered dose inhalers, liquid nebulizers, dry powder inhalers, sprayers, thermal vaporizers, and the like. Other suitable technology for administration of particular 2,4-pyrimidinediamine compounds includes electrohydrodynamic aerosolizers.

In addition, the inhalation device is preferably practical, in the sense of being easy to use, small enough to carry conveniently, capable of providing multiple doses, and durable. Some specific examples of commercially available inhalation devices are Turbohaler (Astra, Wilmington, Del.), Rotahaler (Glaxo, Research Triangle Park, N.C.), Diskus (Glaxo, Research Triangle Park, N.C.), the Ultravent nebulizer (Mallinckrodt), the Acorn II nebulizer (Marquest Medical Products, Totowa, N.J.) the Ventolin metered dose inhaler (Glaxo, Research Triangle Park, N.C.), or the like. In one embodiment, the disclosed compound(s), solvates, N-oxides, pharmaceutically acceptable salts or prodrug(s) thereof can be delivered by a dry powder inhaler or a sprayer.

As those skilled in the art will recognize, the formulation of the form of the disclosed compound(s), solvates, N-oxides, pharmaceutically acceptable salts or prodrug(s) thereof, the quantity of the formulation delivered, and the duration of administration of a single dose depend on the type of inhalation device employed as well as other factors. For some aerosol delivery systems, such as nebulizers, the frequency of administration and length of time for which the system is activated will depend mainly on the concentration of the disclosed compound(s) in the aerosol. For example, shorter periods of administration can be used at higher concentrations the disclosed compound(s) in the nebulizer solution. Devices such as metered dose inhalers can produce higher aerosol concentrations, and can be operated for shorter periods to deliver the desired amount of active compound in some embodiments. Devices such as dry powder inhalers deliver active agent until a given charge of agent is expelled from the device. In this type of inhaler, the amount of the disclosed compound(s), solvates, N-oxides, pharmaceutically acceptable salts or prodrug(s) thereof in a given quantity of the powder determines the dose delivered in a single administration. The formulation of the disclosed compound(s) is selected to yield the desired particle size in the chosen inhalation device.

Formulations of a disclosed compound for administration from a dry powder inhaler may typically include a finely divided dry powder containing the disclosed compound(s), but the powder can also include a bulking agent, buffer, carrier, excipient, another additive, or the like. Additives can be included in a dry powder formulation, for example, to dilute the powder as required for delivery from the particular powder inhaler, to facilitate processing of the formulation, to provide advantageous powder properties to the formulation, to facilitate dispersion of the powder from the inhalation device, to stabilize to the formulation (e.g., antioxidants or buffers), to provide taste to the formulation, or the like. Typical additives include mono-, di-, and polysaccharides; sugar alcohols and other polyols, such as, for example, lactose, glucose, raffinose, melezitose, lactitol, maltitol, trehalose, sucrose, mannitol, starch, or combinations thereof; surfactants, such as sorbitols, diphosphatidyl choline, or lecithin; or the like.

The method of the invention can be conducted a pharmaceutical composition including the disclosed compound(s) suitable for administration by inhalation. For example, a dry powder formulation can be manufactured in several ways, using conventional techniques, such as described in any of the publications mentioned above and incorporated expressly herein by reference, and for example, Baker, et al., U.S. Pat. No. 5,700,904, the entire disclosure of which is incorporated expressly herein by reference. Particles in the size range appropriate for maximal deposition in the lower respiratory tract can be made by micronizing, milling, or the like. And a liquid formulation can be manufactured by dissolving the compound in a suitable solvent, such as water, at an appropriate pH, including buffers or other excipients.

A specific example of an aqueous suspension formulation suitable for nasal administration using commercially-available nasal spray devices includes the following ingredients: active compound or prodrug (0.5 20 mg/ml); benzalkonium chloride (0.1 0.2 mg/mL); polysorbate 80 (TWEEN® 80; 0.5 5 mg/ml);

carboxymethylcellulose sodium or microcrystalline cellulose (1 15 mg/ml); phenylethanol (1 4 mg/ml); and dextrose (20 50 mg/ml). The pH of the final suspension can be adjusted to range from about pH 5 to pH 7, with a pH of about pH 5.5 being typical.

Another specific example of an aqueous suspension suitable for administration of the compounds via inhalation contains 20 mg/mL Compound or prodrug, 1% (v/v) Polysorbate 80 (TWEEN® 80), 50 mM citrate and/or 0.9% sodium chloride.

For ocular administration, the active compound(s) or prodrug(s) may be formulated as a solution, emulsion, suspension, etc. suitable for administration to the eye. A variety of vehicles suitable for administering compounds to the eye are known in the art. Specific non-limiting examples are described in U.S. Pat. Nos. 6,261,547; 6,197,934; 6,056,950; 5,800,807; 5,776,445; 5,698,219; 5,521,222; 5,403,841; 5,077,033; 4,882,150; and 4,738,851, which are incorporated herein by reference.

For prolonged delivery, the disclosed compound(s) can be formulated as a depot preparation for administration by implantation or intramuscular injection. The active ingredient maybe formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the disclosed compound(s) for percutaneous absorption may be used. To this end, permeation enhancers may be used to facilitate transdermal penetration of the active compound(s). Suitable transdermal patches are described in for example, U.S. Pat. Nos. 5,407,713; 5,352,456; 5,332,213; 5,336,168; 5,290,561; 5,254,346; 5,164,189; 5,163,899; 5,088,977; 5,087,240; 5,008,110; and 4,921,475, which are incorporated herein by reference.

Alternatively, other pharmaceutical delivery systems may be employed. Liposomes and emulsions are well-known examples of delivery vehicles that may be used to deliver active compound(s) or prodrug(s). Certain organic solvents, such as dimethylsulfoxide (DMSO), may also be employed, although usually at the cost of greater toxicity. In some embodiments, the disclosed compound(s) as an active ingredient or solvates, N-oxides, pharmaceutically acceptable salts or prodrug(s) thereof, is administered orally in the form of a tablet.

The pharmaceutical compositions may, if desired, be presented in a pack or dispenser device which may contain one or more unit dosage forms containing the active compound(s). The pack may, for example, comprise metal or plastic foil, such as a blister pack. The pack or dispenser device may be accompanied by instructions for administration.

I. Spray-Dried Formulation

Disclosed herein are embodiments of a spray-dried formulation comprising one or more disclosed compounds, such as one or more compounds according to Formula VII. The spray-dried formulation may be a dispersion, such as a spray-dried dispersion of a compound(s) according to Formula VII in a carrier or matrix, such as a polymer matrix. Typically, the spray-dried formulation comprises a single phase, amorphous dispersion of the disclosed compound(s) in the carrier, such as a polymer matrix.

Embodiments of the spray-dried formulation comprise, consist essentially of, or consist of, an effective amount of one or more compounds, such as one or more compounds according to Formula VII, and an amount of the carrier sufficient to form the spray-dried formulation. A person of ordinary skill in the art will appreciate that an effective amount of the compound(s) may vary, but typically the effective amount is from 0.1% to 50% (w/w with respect to the carrier) or more, such as from 1% to 50%, from 5% to 40%, from 10% to 35%, from 15% to 30%, or from 15% to 25%. In particular embodiments, the spray-dried formulation comprises, consists essentially of, or consists of, 20% w/w of the disclosed compound(s) and 80% w/w carrier, such as a polymer matrix.

In some embodiments, the carrier is a polymer, such as a polymer that is suitable to form a spray-dried formulation with the disclosed compound(s). Suitable polymers include, but are not limited to, cellulose derivatives, such as hydroxypropylmethylcellulose acetate succinate (hypromellose acetate succinate; HPMCAS), hydroxypropyl methylcellulose phthalate (hypromellose phthalate; HPMCP) or hydroxypropyl methylcellulose (HPMC); vinyl polymers, such as poly(vinylpyrrolidone) (PVP), or poly(vinylpyrrolidone-co-vinyl acetate) (PVPVA); lactide polymers, such as polylactide (PLA) or polylactide-co-glycolide (PLGA); sugars, such as sucrose or trehalose; or any combination thereof. In certain embodiments, the carrier is HPMCAS. The polymer, such as HPMCAS, may be of any grade suitable to form the spray-dried formulation, such as grade L, grade M, or grade H. In particular embodiments, grade M is used. Additionally, the HPMCAS may be a fine grade (F) or a granular grade (G), and in certain embodiments, fine grade is used. And in certain working embodiments, the carrier is HPMCAS-MF.

In some embodiments, the spray-dried formulation has a suitable glass transition temperature. The glass transition temperature may be from 100° C. or less to 120° C. or more, such as from 105° C. to 110° C. or 107° C. to 110° C. In certain working embodiments, the glass transition temperature is from 108° C. to 109° C.

In some embodiments, the formulation may comprise additional components. Additional components can be included in pharmaceutical compositions for a variety of purposes, such as to dilute a composition for delivery to a subject, to facilitate processing of the formulation, to provide advantageous material properties to the formulation, to facilitate dispersion from a delivery device, to stabilize the formulation (e.g., antioxidants or buffers), to provide a pleasant or palatable taste or consistency to the formulation, or the like. Typical additional components include, by way of example and without limitation: pharmaceutically acceptable excipients; pharmaceutically acceptable carriers; and/or adjuvants, such as mono-, di-, and polysaccharides, sugar alcohols and other polyols, such as, lactose, glucose, raffinose, melezitose, lactitol, maltitol, trehalose, sucrose, mannitol, starch, or combinations thereof; surfactants, such as sorbitols, diphosphatidyl choline, and lecithin; bulking agents; buffers, such as phosphate and citrate buffers; anti-adherents, such as magnesium stearate; binders, such as saccharides (including disaccharides, such as sucrose and lactose,), polysaccharides (such as starches, cellulose, microcrystalline cellulose, cellulose ethers (such as hydroxypropyl cellulose), gelatin, synthetic polymers (such as polyvinylpyrrolidone, polyalkylene gylcols); coatings (such as cellulose ethers, including hydroxypropylmethyl cellulose, shellac, corn protein zein, and gelatin); release aids (such as enteric coatings); disintegrants (such as crospovidone, crosslinked sodium carboxymethyl cellulose, and sodium starch glycolate); fillers (such as dibasic calcium phosphate, vegetable fats and oils, lactose, sucrose, glucose, mannitol, sorbitol, calcium carbonate, and magnesium stearate); flavors and sweeteners (such as mint, cherry, anise, peach, apricot or licorice, raspberry, and vanilla; lubricants (such as minerals, exemplified by talc or silica, fats, exemplified by vegetable stearin, magnesium stearate or stearic acid); preservatives (such as antioxidants exemplified by vitamin A, vitamin E, vitamin C, retinyl palmitate, and selenium, amino acids, exemplified by cysteine and methionine, citric acid and sodium citrate, parabens, exemplified by methyl paraben and propyl paraben); colorants; compression aids; emulsifying agents; encapsulation agents; gums; granulation agents; and combinations thereof.

II. Method of Making a Spray-Dried Formulation

Embodiments of a method for making the spray-dried formulation are also disclosed herein. In some embodiments, one or more compounds, such as one or more compounds according to Formula VII, and the polymer are dissolved in a suitable solvent or mixture of solvents, and then spray-dried. Suitable solvent(s) include any solvent or mixture of solvents that dissolves the disclosed compound(s) and the carrier and is suitable for a spray-drying process. Exemplary solvents include, but are not limited to, alcohol, such as methanol, ethanol, isopropanol, n-propanol, and the like; chlorinated solvents, such as dichloromethane, chloroform. In some embodiments, the disclosed compound(s) is dissolved in the solvent or mixture of solvents, and the polymer is added to the mixture. However, in other embodiments, the polymer is dissolved first and the compound(s) is subsequently added, or the compound(s) and the polymer are mixed substantially simultaneously with the solvent or solvent mixture. Regardless of the order of addition, the mixture typically is mixed until the disclosed compound(s) and the polymer are dissolved, and/or the mixture has a uniform appearance. In some embodiments, the resulting mixture is stored at a reduced temperature, such as below 25° C., or from less than 25° C. to 0° C., from 15° C. to 0° C., from 10° C. to 0° C., or from 7° C. to 3° C., typically at about 5° C. The solution also may be protected from light, i.e. stored in a dark environment.

The solution is then spray-dried using a spray drying apparatus. Suitable spray-drying apparatuses are known to persons of ordinary skill in the art. In some embodiments, the parameters of the spray drying apparatus, such as feed temperature, inlet temperature, target outlet temperature and aspiration are set to values suitable for the disclosed compound(s) and the polymer, as understood by a person of ordinary skill in the art. In certain embodiments, the feed temperature is from 15° C. or less to 35° C. or more, such as from 20° C. to 25° C. The inlet temperature may be from 40° C. or less to 60° C. or more, such as from 45° C. to 55° C. The target outlet temperature may be from 30° C. or less to 45° C. or more, such as from 32° C. to 42° C. or from 34° C. to 40° C. And/or the aspirator may be from 50% or more to 100%, such as from 70% to 100% or from 80% to 100%.

The resulting spray-dried solid may be further dried at a temperature suitable to remove at least some, and may be substantially all, of any remaining solvent without substantially degrading the disclosed compound(s) and/or the carrier. In some embodiments, the solid is dried at a temperature of from 25° C. to 100° C. or more, such as from 30° C. to 75° C., or from 35° C. to 50° C. The dispersion may be dried until substantially all the remaining solvent has been removed, and/or until no further weight loss is achieved. The drying may continue for from 1 hour to 48 hours or more, such as from 6 hours to 36 hours, from 12 hours to 32 hours, or from 18 hours to 24 hours. The resulting solid formulation may be stored at a reduced temperature, such as such as below 25° C., or from less than 25° C. to 0° C., from 15° C. to 0° C., from 10° C. to 0° C., or from 7° C. to 3° C., typically at about 5° C. The solution also may be protected from light, i.e. stored in a dark environment, and/or stored under dry conditions, such as in the presence of a desiccant and/or under a dry atmosphere.

### VII. Dosages

The disclosed compound(s) or a composition thereof, will generally be used in an amount effective to achieve a desired result, for example, in an amount effective to treat or prevent CRS. The compound(s), or compositions thereof, can be administered therapeutically to achieve a therapeutic benefit and/or prophylactically to achieve a prophylactic benefit. Therapeutic benefit means eradication or amelioration of the underlying CRS and/or eradication or amelioration of one or more of the symptoms associated with CRS, such that the patient reports an improvement in feeling or condition, notwithstanding that the patient may still be afflicted with CRS. In some embodiments, indicators of therapeutic improvement and/or successful treatment may include preventing the subject from exhibiting one or more symptoms at a relevant score on the CRS grading scale, such as preventing a subject from exhibiting grade 2 or higher CRS. Additionally, or alternatively, an indicator of therapeutic improvement and/or successful treatment may be a change in grading or severity on the grading scale as discussed herein, such as a change from a score of 4 to a score of 3 or lower, or a change from a score of 3 to a score of 2 or 1. A prophylactic benefit may be achieved by substantially preventing CRS from developing, such as preventing the onset of any symptoms, or preventing one or more symptoms from progressing above grade 1. In some embodiments, prophylactic benefit may mean preventing the subject from exhibiting one or more symptoms at a level of grade 2 or higher. As known by those of ordinary skill in the art, the preferred dosage of the compound(s) also will depend on various factors, including the age, weight, general health, and severity of the condition of the patient or subject being treated. Dosage also may need to be tailored to the sex of the individual and/or the lung capacity of the individual, when administered by inhalation. Dosage also may be tailored to individuals suffering from more than one condition or those individuals who have additional conditions that affect lung capacity and the ability to breathe normally, for example, emphysema, bronchitis, pneumonia, and respiratory infections. Dosage, and frequency of administration of the disclosed compound(s) or compositions thereof, will also depend on whether the compound(s) are formulated for treatment of acute episodes of CRS or for the prophylactic treatment of CRS. A person or ordinary skill in the art will be able to determine the optimal dose for a particular individual.

The disclosed compound(s), or compositions thereof, can be administered before, during, and/or after therapy that can induce CRS. In one embodiment, the disclosed compound(s), or compositions thereof, is administered within 48 hours before therapy that can induce CRS is to begin, such as within 24, 12, 6, 4, or 2 hours of the therapy. In another embodiment, the disclosed compound(s), or compositions thereof, can be administered during the course of the therapy. In another embodiment the disclosed compound(s), or compositions thereof, can be administered following completion of the therapy, either immediately or shortly following completion of the therapy (e.g., within 24, 48, 72 or 96 hours or 1 week of the completion of therapy). In another embodiment, the disclosed compound(s), or compositions thereof, can be administered during two or more of the time periods consisting of before, during, or after the therapy.

For prophylactic administration, the disclosed compound(s), or compositions thereof, can be administered to a patient or subject at risk of developing CRS. For example, a compound(s), or composition thereof, can be administered to a subject prior to the start of a treating likely to cause CRS, substantially simultaneously with the onset of such a treatment, or subsequent to the treatment being initiated. A compound(s), or compositions thereof, also can be administered prophylactically to individuals who may be repeatedly treated by a treatment that has caused CRS in other individually, even if the subject previously has not developed CRS.

Effective dosages can be estimated initially from in vitro assays. For example, an initial dosage for use in subjects can be formulated to achieve a circulating blood or serum concentration of active compound that is at or above an IC₅₀ or EC₅₀ of the particular compound as measured in an in vitro assay. Dosages can be calculated to achieve such circulating blood or serum concentrations taking into account the bioavailability of the particular compound. Fingl & Woodbury, “General Principles,” In: Goodman and Gilman's The Pharmaceutical Basis of Therapeutics, Chapter 1, pages 1-46, Pergamon Press, and the references cited therein, provide additional guidance concerning effective dosages.

In some embodiments, the disclosed compounds have an EC₅₀ from greater than 0 to 20 μM, such as from greater than 0 to 10 μM, from greater than 0 to 5 μM, from greater than 0 to 1 μM, from greater than O to 0.5 μM, or from greater than 0 to 0.1 μM.

Initial dosages can also be estimated from in vivo data, such as animal models, including mouse and non-human primate models. CRS animal models are known to persons of ordinary skill in the art, and additional information may be found in Norelli, M., Camisa, B., Barbiera, G. et al. Monocyte-derived IL-1 and IL-6 are differentially required for cytokine-release syndrome and neurotoxicity due to CAR T cells. Nat Med. 2018; 24: 739-748, and Giavridis, T., van der Stegen, S. J. C., Eyquem, J., Hamieh, M., Piersigilli, A., and Sadelain, M. CAR T cell-induced cytokine release syndrome is mediated by macrophages and abated by IL-1 blockade. Nat Med. 2018; 24: 731-738

Dosage amounts of disclosed compounds will typically be in the range of from about greater than O mg/kg/day, such as 0.0001 mg/kg/day or 0.001 mg/kg/day or 0.01 mg/kg/day, up to at least about 1000 mg/kg/day, such as up to 100 mg/kg/day, but can be higher or lower, depending upon, among other factors, the activity of the compound, its bioavailability, the mode of administration and various factors discussed herein. More typically, the dosage (or effective amount) may range from about 0.0025 mg/kg to about 1 mg/kg administered at least once per day, such as from 0.01 mg/kg to about 0.5 mg/kg or from about 0.05 mg/kg to about 0.15 mg/kg. The total daily dosage typically ranges from about 0.1 mg/kg to about 5 mg/kg or to about 20 mg/kg per day, such as from 0.5 mg/kg to about 10 mg/kg per day or from about 0.7 mg/kg per day to about 2.5 mg/kg/day. Dosage amounts can be higher or lower depending upon, among other factors, the activity of the compound, its bioavailability, the mode of administration, and various factors discussed above.

Dosage amount and dosage interval can be adjusted for individuals to provide plasma levels of the compound(s) that are sufficient to achieve and/or maintain a desired therapeutic or prophylactic effect. For example, the compounds can be administered once per day, multiple times per day, once per week, multiple times per week (e.g., every other day), one per month, multiple times per month, or once per year, depending upon, amongst other things, the mode of administration, the specific indication being treated, and the judgment of the prescribing physician. Persons of ordinary skill in the art will be able to optimize effective local dosages without undue experimentation. In some embodiments, the amount of the disclosed compound in a composition to be administered, or the amount of the compound to be administered in a method disclosed herein, is a suboptimal dose. As used herein, a suboptimal dose is a dose typically used in a single administration to a patient in monotherapy or in standard of care combination therapies.

Compositions comprising one or more of the disclosed compounds typically comprise from greater than 0 up to 99% of the compound, or compounds, and/or other therapeutic agent by total weight percent. More typically, compositions comprising one or more of the disclosed compounds comprise from about 1 to about 20 total weight percent of the compound and other therapeutic agent, and from about 80 to about 99 weight percent of a pharmaceutically acceptable additive.

Preferably, the compound(s), or compositions thereof, will provide therapeutic or prophylactic benefit without causing substantial toxicity. Toxicity of the compound can be determined using standard pharmaceutical procedures. The dose ratio between toxic and therapeutic (or prophylactic) effect is the therapeutic index. Compounds that exhibit high therapeutic indices are preferred.

### VIII. EXAMPLES

### Example 1

### Synthesis of pyrimidine-2,4-diamines

Synthesis of 5-(2-chloro-5-methylpyrimidin-4-ylamino)benzo[d]oxazol-2(3H)-one: To a vial with 5-aminobenzo[d]oxazol-2(3H)-one (300.1 mg, 2.0 mmol) and 2,4-dichloro-5-methylpyrimidine (423.8 mg, 2.6 mmol), MeOH (8 mL) and H₂O (2 mL) were added. The turbid mixture was stirred at room temperature for 64 h. Precipitate from reaction mixture was collected by filtration, washing with EtOAc (3 mL×2), and was further dried in vacuo. 5-(2-Chloro-5-methylpyrimidin-4-ylamino)benzo[d]oxazol-2(3H)-one was obtained as an off-white solid: 394 mg (71% yield); ¹H NMR (300 MHz, DMSO) δ 11.68 (br s, 1H), 8.62 (s, 1H), 7.94 (d, J=0.8, 1H), 6.97 (d, J=2.0, 1H), 6.82 (d, J=8.1, 1H), 6.74 (dd, J=2.0, 8.1, 1H), 2.15 (s, 3H); LCMS (M+) m/z 277.10.

Synthesis of N4-(benzo[d]oxazol-2(3H)-on-5-yl)-N2-(3-methylsulfonyl)phenyl)-5-methylpyrimidine-2,4-diamine: (I-16)

To a vial with 5-(2-chloro-5-methylpyrimidin-4-ylamino)benzo[d]oxazol-2(3H)-one (138.3 mg, 0.5 mmol) and 3-(methylsulfonyl)benzenamine hydrochloride (207.7 mg, 1.0 mmol), i—PrOH (10 mL) was added, followed by TFA (116 pi, 1.5 mmol). The vial was tightly closed, and the reaction mixture was stirred at 85-90° C. for 40 h. The solvent was removed in vacuo, and the crude product was purified by RP-HPLC. N4-(benzo[d]oxazol-2(3H)-on-5-yl)-N2-(3-methylsulfonyl)phenyl)-5-methylpyrimidine-2,4-diamine was obtained as a mono-trifluoroacetate salt: an off-white solid, 129 mg (49% yield); ¹H NMR (300 MHz, DMSO) δ 11.60 (s, 1H), 9.43 (s, 1H), 8.43 (s, 1H), 8.20 (s, 1H), 8.11 (br d, J=7.5, 1H), 7.96 (d, J=0.8, 1H), 7.49-7.33 (m, 4H), 7.27 (d, J=8.5, 1H), 3.13 (s, 3H), 2.16 (s, 3H); LCMS (M+) m/z 412.47.

Synthesis of 5-(2-chloro-5-fluoropyrimidin-4-ylamino)-1H-benzo[d]imidazol-2(3H)-one:

To a vial with 5-amino-1H-benzokilimidazol-2(3H)-one (298.3 mg, 2.0 mmol) and 2,4-dichloro-5-fluoropyrimidine (434.1 mg, 2.6 mmol), MeOH (8 mL) and H₂O (2 mL) were added. The turbid solution was stirred at rt for 3 days. Precipitate from reaction mixture was collected by filtration, and washing with EtOAc (3 mL×2), and was further dried in vacuo. 5-(2-Chloro-5-fluoropyrimidin-4-ylamino)-1H-benzokilimidazol-2(3H)-one was obtained as an off-white solid: 390.3 mg (70% yield); ¹H NMR (300 MHz, DMSO) δ 10.69 (s, 1H), 10.63 (s, 1H), 9.87 (s, 1H), 8.27 (d, J=3.6, 1H), 7.35 (d, J=1.9, 1H), 7.18 (dd, J=1.9, 8.3, 1H), 6.93 (d, J=8.3, 1H); LCMS (M+) m/z 279.80.

Synthesis of 4-(5-nitropyridin-2-yl)morpholine:

In a round-bottom flask, to a dichloromethane (125 mL) solution of 2-bromo-5-nitropyridine (5 g, 24.6 mmol), morpholine (5.4 mL, 61.5 mmol) was added. The reaction was refluxed for 4 hr, then cooled to room temperature. The solution was subsequently washed with saturated aqueous sodium bicarbonate solution and brine. The organic layer was dried (Na₂SO₄), filtered, and the solvent was removed in vacuo. 4-(5—Nitropyridin-2-yl)morpholine, a yellow solid, was obtained: 4.9 g (95% yield); ¹H NMR (300 MHz, DMSO) δ 8.95 (d, J=2.7, 1H), 8.22 (dd, J=2.7, 9.6, 1H), 6.92 (d, J=9.6, 1H), 3.74-3.65 (m, 8H); LCMS (M+) m/z 210.34.

Synthesis of 6-morpholinopyridin-3-amine:

Into a EtOH (250 mL) solution of 4-(5-nitropyridin-2-yl)morpholine (4.9 g, 23.4 mmol), 10% Pd on activated carbon, 500 mg, was added. Hydrogenation was carried out in a Parr flask at room temperature, at 40 psi for 2 hr. The solids were filtered off and the filtrate was collected. The solvent was removed in vacuo. 6-Morpholinopyridin-3-amine, as a purple solid, was obtained: 3.7 g (88% yield); ¹H NMR (300 MHz, DMSO) δ 7.64 (d, J=2.7, 1H), 6.96 (dd, J=2.7, 8.8, 1H), 6.65 (d, J=8.8, 1H), 4.63 (s, 2H), 3.72-3.69 (m, 4H), 3.21-3.18 (m, 4H); LCMS (M+) m/z 180.08.

Synthesis of N4-(benzimidazolin-2-on-5-yl)-N2-((2-morpholinyl)pyridin-5-yl)-5-fluoropyrimidine-2,4-diamine: (II-19)

To a vial with 5-(2-chloro-5-fluoropyrimidin-4-ylamino)-1H-benzokilimidazol-2(3H)-one (27.6 mg, 0.1 mmol) and 6-morpholinopyridin-3-amine (35.8 mg, 0.2 mmol), i—PrOH (2 mL) was added, followed by TFA (10 μL, 0.13 mmol). The vial was tightly closed, and the turbid solution was stirred at 95° C. for 2 days. The solvent was removed in vacuo, and the crude product was purified by RP-HPLC. N4-(Benzimidazolin-2-on-5-yl)-N2-((2-morpholinyl)pyridin-5-yl)-5-fluoropyrimidine-2,4-diamine was obtained as a light orange solid, as a di-trifluoroacetate salt: 51.1 mg (79% yield); ¹H NMR (300 MHz, DMSO) 6 10.61 (s, 2H), 9.79 (br s, 1H), 9.55 (br s, 1H), 8.31 (s, 1H), 8.13 (d, J=4.4, 1H), 7.92 (br d, J=8.8, 1H), 7.22 (d, J=8.1, 1H), 7.18 (s, 1H), 7.10 (br d, J=8.8, 1H), 6.89 (d, J=8.1, 1H), 3.78-3.75 (m, 4H), 3.50-3.47 (m, 4H); LCMS (M+) m/z 423.00.

Synthesis of 6-(2-chloro-5-methylpyrimidin-4-ylamino)benzo[d]oxazol-2(3H)-one:

To a vial with 6-aminobenzo[d]oxazol-2(3H)-one (1.0 g, 6.7 mmol) and 2,4-dichloro-5-methylpyrimidine (1.4 g, 8.7 mmol), solvents MeOH (20 mL) and H₂O (5 mL) were added. The turbid mixture was stirred at room temperature for 2 days. Precipitate from the reaction mixture was collected by filtration, washing with H₂O (3 mL×2) and EtOAc (3 mL×2), and was further drying in vacuo. 6-(2-Chloro-5-methylpyrimidin-4-ylamino)benzo[d]oxazol-2(3H)-one was obtained as a light tan color solid: 1.59 g (86% yield); ¹H NMR (300 MHz, DMSO) δ 11.59 (s, 1H), 8.87 (s, 1H), 7.99 (s, 1H), 7.56 (s, 1H), 7.28 (d, J=8.3, 1H), 7.06 (d, J=8.3, 1H), 2.14 (s, 3H).

Synthesis of N4-(benzo[d]oxazol-2(3H)-on-6-yl)-N2-((3-morpholinyl)phenyl)-5-methylpyrimidine-2,4-diamine: (1-48)

To a vial with 6-(2-chloro-5-methylpyrimidin-4-ylamino)benzo[d]oxazol-2(3H)-one (27.7 mg, 0.1 mmol) and 3-morpholinobenzenamine (26.7 mg, 0.15 mmol), i—PrOH (2 mL) was added, followed by TFA (10 μL,μ, 0.13 mmol). The vial was tightly closed, and the solution was stirred at 95° C. for 2 days. The solvent was removed in vacuo, and the crude product was purified by RP-HPLC. N4-(benzo[d]oxazol-2(3H)-on-6-yl)-N2-((3-morpholinyl)phenyl)-5-methylpyrimidine-2,4-diamine was obtained as a light tan color solid: 32.9 mg (78% yield); ¹H NMR (300 MHz, DMSO) δ 11.57 (s, 1H), 8.97 (s, 1H), 8.46 (s, 1H), 7.90 (s, 1H), 7.81 (s, 1H), 7.37 (d, J=8.3, 1H), 7.27 (s, 1H), 7.14 (d, J=8.3, 1H), 7.07-7.01 (m, 2H), 6.58-6.50 (m, 1H), 3.68-3.65 (m, 4H), 2.95-2.92 (m, 4H), 2.14 (s, 3H); LCMS (M+) m/z 419.03.

Synthesis of N4-(benzoxazolin-2-on-5-yl)-N2-[2-(4-methylpiperazin-1-yl)pyridin-5-yl]-5-methylpyrimidine-2,4-diamine: (II-13)

To a vial with 5-(2-chloro-5-methylpyrimidin-4-ylamino)benzo[d]oxazol-2(3H)-one (27.7 mg, 0.5 mmol) and 6-(4-methylpiperazin-1-yl)pyridin-3-amine (38.4 mg, 1.0 mmol), i—PrOH (2 mL) was added, followed by TFA (10 4, 0.13 mmol). The vial was tightly closed, and the reaction mixture was stirred at 85° C. for 2 days. The solvents were removed in vacuo, and the crude product was purified by RP-HPLC. Purified compound (as a trifluoroacetate salt) was dissolved in MeOH-H₂O (1:4, 2 mL) and was passed through a PL-HCO₃-MP—SPE column, washing with same solvents (1 mL). The filtrate was collected and the solvent was removed by lyophilization. N4-(benzoxazolin-2-on-5-yl)-N2-[2-(4-methylpiperazin-1-yl)pyridin-5-yl]-5-methylpyrimidine-2,4-diamine was obtained as a purple solid, 23.1 mg (53% yield); ¹H NMR (300 MHz, DMSO) δ 11.57 (s, 1H), 8.73 (s, 1H), 8.30-8.28 (m, 2H), 7.87-7.84 (m, 2H), 7.46-7.28 (m, 2H), 7.22 (d, J=8.5, 1H), 6.71 (d, J=9.1, 1H), 3.40-3.37 (m, 4H, overlapped with H₂O), 2.44-2.41 (m, 4H), 2.25 (s, 3H), 2.11 (s, 3H); LCMS (M+) m/z 433.52.

Synthesis of N4-(benzimidazolin-2-on-5-yl)-N2-[2-(4-methylpiperazin-1-yl)pyridin-5-yl]-5-fluoropyrimidine-2,4-diamine: (II-16)

To a vial with 5-(2-chloro-5-fluoropyrimidin-4-ylamino)-1H-benzo[d]imidazol-2(3H)-one (28.0 mg, 0.1 mmol) and 6-(4-methylpiperazin-1-yl)pyridin-3-amine (38.4 mg, 0.2 mmol), i—PrOH (2 mL) was added, followed by TFA (10 4, 0.13 mmol). The vial was tightly closed, and the solution was stirred at 85° C. for 2 days. The solvent was removed in vacuo, and the crude product was purified by RP-HPLC. Purified compound (as a trifluoroacetate salt) was dissolved in MeOH-H₂O (1:4, 2 mL) and was passed through a PL-HCO₃-MP—SPE column, washing with same solvents (1 mL). The filtrate was collected and the solvent was removed by lyophilization. N4-(Benzimidazolin-2-on-5-yl)-N2-[2-(4-methylpiperazin-1-yl)pyridin-5-yl]-5-fluoropyrimidine-2,4-diamine was obtained as a purple solid: 26.2 mg (60% yield); ¹H NMR (300 MHz, DMSO) δ 10.56 (s, 1H), 10.52 (s, 1H), 9.16 (s, 1H), 8.85 (s, 1H), 8.27 (br d, J=2.3, 1H), 8.00 (br d, J=3.8, 1H), 7.84 (dd, J=2.3, 9.1, 1H), 7.30 (dd, J=1.7, 8.2, 1H), 7.17 (d, J=1.7, 1H), 6.86 (d, J=8.2, 1H), 6.73 (d, J=9.1, 1H), 3.40-3.37 (m, 4H, overlapped with H₂O), 2.44-2.41 (m, 4H), 2.25 (s, 3H); LCMS (M+) m/z 436.50.

Synthesis of 6-(5-methyl-2-(6-(4-methylpiperazin-1-yl)pyridin-3-ylamino)pyrimidin-4-ylamino)benzo[d]oxazol-2(3H)-one: (

II-25)

To a vial with 5-(2-chloro-5-fluoropyrimidin-4-ylamino)-1H-benzo[d]imidazol-2(3H)-one (28.0 mg, 0.1 mmol) and 6-(4-methylpiperazin-1-yl)pyridin-3-amine (38.4 mg, 0.2 mmol), i—PrOH (2 mL) was added, followed by TFA (10 4, 0.13 mmol). The vial was tightly closed, and the solution was stirred at 85° C. for 2 days. The solvent was removed in vacuo, and the crude product was purified by RP-HPLC. Purified compound (as a trifluoroacetate salt) was dissolved in MeOH-H₂O (1:4, 2 mL) and was passed through a PL-HCO₃-MP—SPE column, washing with same solvents (1 mL). The filtrate was collected and the solvent was removed by lyophilization. 6-(5-Methyl-2-(6-(4-methylpiperazin-1-yppyridin-3-ylamino)pyrimidin-4-ylamino)benzo[d]oxazol-2(3H)-one was obtained as a purple solid: 26.2 mg (60% yield); ¹H NMR (300 MHz, DMSO) δ 11.70 (s, 1H), 9.97 (s, 1H), 9.49 (s, 1H), 8.15 (s, 1H), 7.82 (s, 1H), 7.65 (d, J=11.7, 2H), 7.21 (d, J=8.8, 1H), 7.05 (d, J=8.4, 1H), 6.89 (d, J=8.9, 1H), 4.34-4.31 (m, 4H), 3.09-3.07 (m, 4H), 2.85 (s, 3H), 2.13 (s, 3H).

Compounds I-1 to 1-538, II-1 to

II-153, III-1 to III-14 and IV-1 to IV-64 were made by methods similar to those described herein and/or known to persons of ordinary skill in the art. Additional information concerning these compounds can be found in International publication No. WO2010/085684, which is incorporated herein by reference in its entirety.

### Example 2

### Synthesis of Pyrazole Compounds

### Preparation of Amine 106:

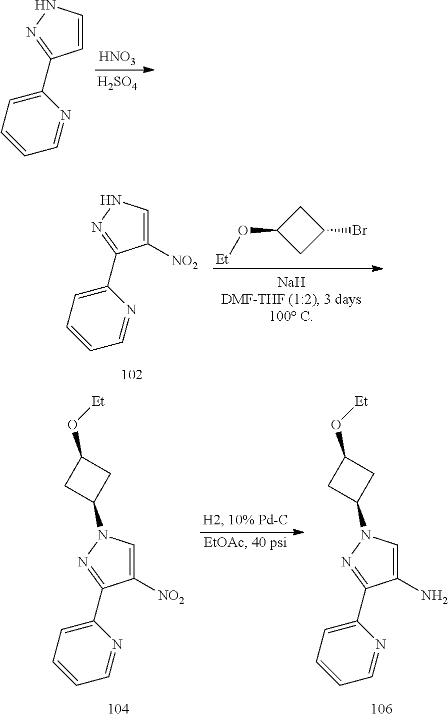

2-(1H-Pyrazol-3-yl)pyridine (10 g) was suspended in concentrated sulfonic acid (30 mL), then fuming nitric acid (6.5 mL, 2 eq.) was added to the solution dropwise while stirring. The reaction mixture was stirred overnight at room temperature. It was quenched by pouring into ice-water (500 mL). The aqueous solution was neutralized by adding solid sodium carbonate, until pH reached around 8. White precipitate was collected by filtration, washed with water and dried to give 2-(4-nitro-1H-pyrazol-3-yl)pyridine 102 (13 g, 99% yield).

2-(4-nitro-1H-pyrazol-3-yl)pyridine 102 (2 g), and 1-bromo-3-ethoxycyclobutane (90% trans isomer, 2 g) were suspended in THF (20 mL) and DMF (10 mL). Sodium hydride (60% in oil, 670 mg, 1.5 eq.) was added to the reaction. The reaction solution was heated at 100° C. for 3 days and then was evaporated. The residue was purified by combiflash chromatography (EtOAc in hexanes=10-100%) to give product 104.

Compound 104 was dissolved in EtOAc (100 mL) and charged with 10% Pd-C catalyst (200 mg). The reaction mixture was shaken under 40 psi hydrogen for 1 hour. LC-MS indicated fully reduction of nitro group. The catalyst was filtered off through celite and washed with EtOAc (5×20 mL). The filtrate was concentrated to give amine 106 (1.4 g, 52% yield in two steps).

### Exemplary synthesis of V-28: N-(1-((1,3-cis)-3-ethoxycyclobutyl)-3-(pyridin-2-yl)-1H-pyrazol-4-yl)-5-(1H-pyrazol-4-yl)furan-2-carboxamide.

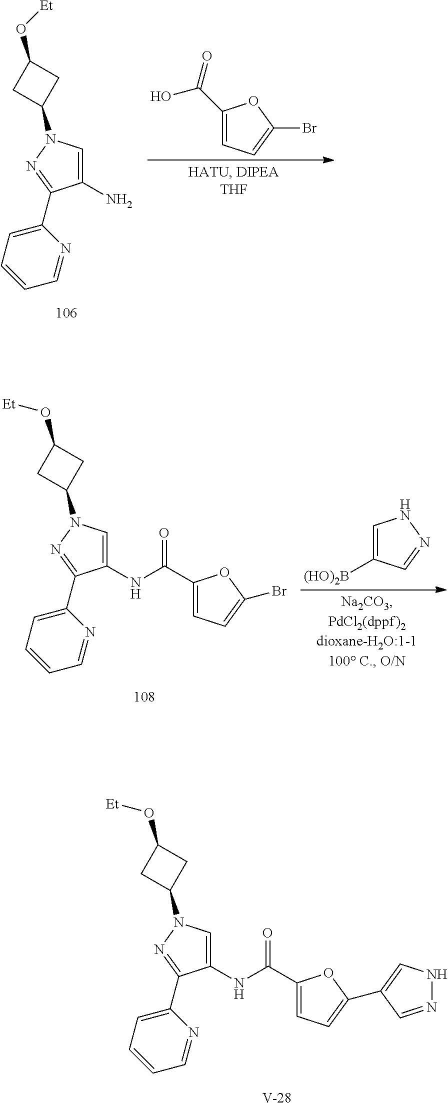

Compound 106 (700 mg), 5-bromo-2-furoic acid (622 mg, 1.2 eq.), and 1-[bis(dimethylamino)methylene]-1-1H-1,2,3-triazolo[4,5-b]pyridinium 3-oxid hexafluorophosphate (HATU) (1.54 g, 1.5 eq.) were dissolved in THF (30 mL) and diisopropylethylamine (DIPEA) (0.7 mL, 1.5 eq.) was added to the solution. The reaction mixture was stirred at room temperature overnight and evaporated. The residue was purified by combiflash chromatography (EtOAc in hexanes=10-100%) to give product 108 (1 g, 87% yield).

Compound 108 (1g), pyrazole-4-boronic acid (780 mg, 3 eq.), Na₂CO₃ (2.45 g, 10 eq.) and PdCl₂ (dppf)₂ (250 mg) were stirred in dioxane (15 mL) and water (15 mL). The reaction mixture was heated at 100° C. overnight. LC-MS indicated fully conversion to the product. The reaction mixture was evaporated and purified by combiflash chromatography (2.0 M NH_{3/}MeOH in DCM=0-20%) to give desired product V-28 (750 mg, 77% yield). ¹H NMR (300 MHz, DMSO) δ 13.25 (br, 1H), 11.63 (s, 1H), 8.72 (dd, J=6.0 Hz, 1H), 8.39 (s, 1H), 8.25 (s, 1H), 8.06 (d, J=6.9 Hz, 1H), 7.95 (m, 2H), 7.42 (m, 1H), 7.26 (d, J=3.9 Hz, 1H), 6.77 (d, J=3.3 Hz, 1H), 4.60 (p, J=7.8 Hz, 1H), 3.83 (p, J=7.5 Hz, 1H), 3.40 (q, J=6.9 Hz, 2H), 2.79 (m, 2H), 2.41 (m, 2H), 1.13 (t, J=6.9 Hz, 3H); LCMS: purity: 100%; MS (m/e): 419.60 (MH+).

### Preparation of 2-methyl-1-(4-nitro-3-(pyridin-2-yl)-1H-pyrazol-1-yl)propan-2-ol (110).

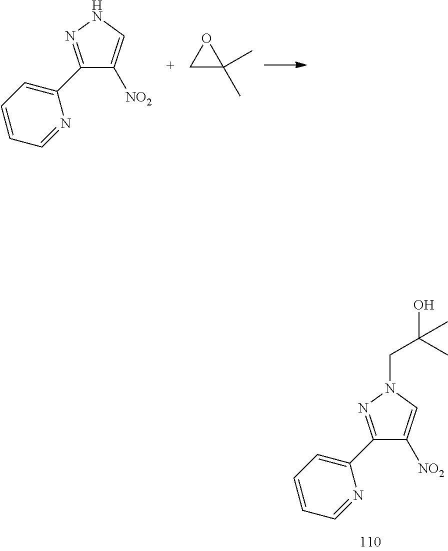

Sodium hydride (1.657 g, 41.4 mmol) was weighed out and added to a dry reaction tube with magnetic stir bar and cooled to 0° C. This was carefully suspended in 86 mL THF and the system was purged with nitrogen. 2-(4—Nitro-1H-pyrazol-3-yl)pyridine (3.928 g, 20.7 mmol) was added in 40 mL dimethylformamide followed by 7 mL dimethylformamide washings. This was stirred 30 minutes at 0° C. followed by 30 minutes at room temperature. It was then cooled back to 0° C. and isobutylene oxide (5.5 mL, 61.9 mmol) was added. The reaction was stirred warming to room temperature, heated 3 hours at 100° C. and stirred overnight at room temperature. The reaction was recharged with sodium hydride (0.445 g, 11.2 mmol) and isobutylene oxide (1.8 mL, 20.3 mmol) and heated 2 hours more at 100° C. The reaction was quenched with water and concentrated to dryness; the residue was partitioned between saturated aqueous sodium bicarbonate and ethyl acetate. The aqueous layer was extracted three times more with ethyl acetate and the combined organic layer was washed with brine and dried over sodium sulfate. Product solution was filtered, concentrated onto silica and purified by column chromatography. After drying, 1.92 g of the title compound 110 was obtained in two batches (35% yield).

¹H NMR (300 MHz, DMSO-d₆) δ 8.73 (s, 1H), 8.72-8.45 (m, 1H), 7.95-7.88 (m, 1H), 7.71-7.65 (m, 1H), 7.51-7.43 (m, 1H), 4.89 (s, 1H), 4.14 (s, 2H), 1.14 (s, 6H). m/z=263 (M+H)⁺.

### Preparation of 1-(4-amino-3-(pyridin-2-yl)-1H-pyrazol-1-yl)-2-methylpropan-2-ol 112.

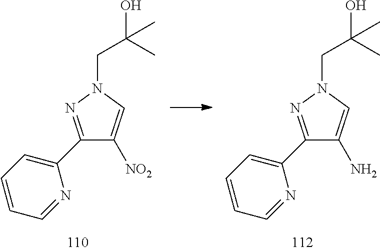 [SMILES: CC(C)(O)Cn1cc([N+](=O)[O-])c(-c2ccccn2)n1 ; CC(C)(O)Cn1cc(N)c(-c2ccccn2)n1]

2-Methyl-1-(4-nitro-3-(pyridin-2-yl)-1H-pyrazol-1-yl)propan-2-ol 110 (0.994 g, 3.8 mmol) was added to a Parr reaction bottle in 100 mL ethyl acetate. This was put under nitrogen and charged with (wet) 10% Pd on carbon (0.404 g, 0.2 mmol). This was run at 60 psi hydrogen overnight on the Parr hydrogenator. The reaction was filtered through Celite with methanol washings, concentrated onto silica and purified by column chromatography. 0.723 g of the title compound 112 was obtained after drying on high vacuum (82% yield).

¹H NMR (300 MHz, DMSO-d₆) δ 8.51 (ddt, J=5.0, 1.9, 0.9 Hz, 1H), 7.85-7.71 (m, 2H), 7.23-7.11 (m, 2H), 4.98 (s, 2H), 4.68 (s, 1H), 3.92 (s, 2H), 1.08 (s, 6H). m/z=233 (M+H)⁺.

### Preparation of 5-bromo-N-(1-(2-hydroxy-2-methylpropyl)-3-(pyridin-2-yl)-1H-pyrazol-4-yl)furan-2-carboxamide 114.

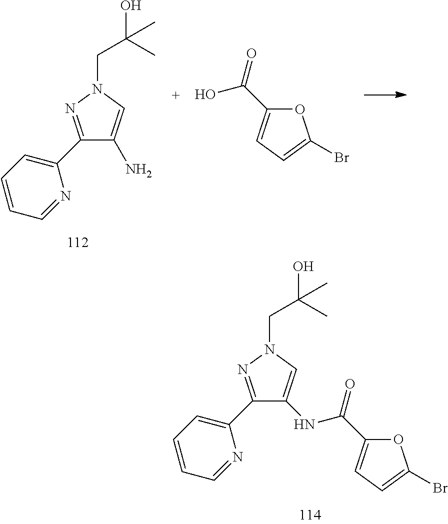 [SMILES: CC(C)(O)Cn1cc(N)c(-c2ccccn2)n1 ; O=C(O)c1ccc(Br)o1 ; CC(C)(O)Cn1cc(NC(=O)c2ccc(Br)o2)c(-c2ccccn2)n1]

5-Bromofuran-2-carboxylic acid (0.148 g, 0.77 mmol) was weighed out and added to a flask with magnetic stir bar. This was dissolved in 33 mL dichloromethane and diisopropylethylamine (0.20 mL, 1.2 mmol) was added followed by HATU (0.381 g, 1.0 mmol). This is stirred 30 minutes at room temperature and 1-(4-amino-3-(pyridin-2-yl)-1H-pyrazol-1-yl)-2-methylpropan-2-ol 112 (0.214 g, 0.92 mmol) was added in 13 mL dichloromethane solution. The reaction was stirred overnight at room temperature. This was concentrated directly onto silica and purified by column chromatography. After drying, 0.358 g of the title compound 114 was obtained. (96% mass balance based on the aminopyrazole; hydroybutyl-related byproducts remained in the purified product. This was used directly.)

¹H NMR (300 MHz, DMSO-d₆) δ 11.82 (s, 1H), 8.65 (ddd, J=5.0, 1.8, 1.0 Hz, 1H), 8.34 (s, 1H), 8.02-7.90 (m, 2H), 7.41 (ddd, J=7.2, 5.0, 1.6 Hz, 1H), 7.27 (d, J=3.6 Hz, 1H), 6.88 (d, J=3.6 Hz, 1H), 4.77 (s, 1H), 4.11 (s, 2H), 1.12 (s, 6H). m/z=405/407 (M+H)⁺(bromine isotopes).

### Preparation of V-1: N-(1-(2-hydroxy-2-methylpropyl)-3-(pyridin-2-yl)-1H-pyrazol-4-yl)-5-(1-methyl-1H-pyrazol-4-yl)furan-2-carboxamide.

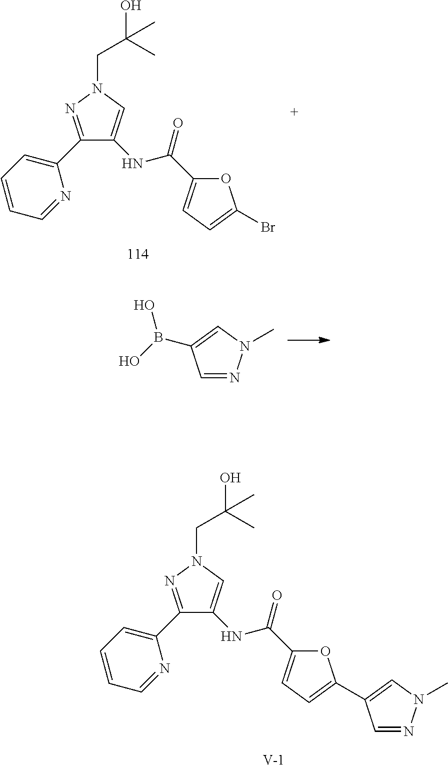

5-bromo-N-(1-(2-hydroxy-2-methylpropyl)-3-(pyridin-2-yl)-1H-pyrazol-4-yl)furan-2-carboxamide 114 (49 mg, 0.12 mmol) in 1.7 mL premixed 7/3 dimethoxyethane/ethanol solution was added to a microwave reaction vial with magnetic stir bar. (1-Methyl-1H-pyrazol-4-yl)boronic acid (99 mg, 0.78 mmol) was weighed out and added to the vial. 2M aqueous sodium carbonate solution (0.41 mL, 0.82 mmol) was added and the reaction was subjected to vigorous subsurface nitrogen sparge. Pd[P(Ph)₃]₂Cl₂(16 mg, 0.02 mmol) was added, the tube was sealed under nitrogen and then heated 30 minutes in the microwave at 130° C. The reaction was worked up in the tube, first diluting with ethyl acetate. This was washed in succession with brine, 1M aqueous sodium hydroxide solution, and brine, pipetting the aqueous layer off the bottom of the tube. The aqueous was back-extracted twice with ethyl acetate and the combined organic layer was dried in a vial over sodium sulfate. The product solution was filtered into another vial, evaporated, and purified by preparative HPLC. After drying, 6 mg of the title compound V-1 was obtained as the TFA salt (10% yield; an additional 12 mg less pure product was recovered).

¹H NMR (300 MHz, DMSO-d₆) δ 11.65 (s, 1H), 8.75 (ddd, J=5.0, 1.8, 0.9 Hz, 1H), 8.38 (s, 1H), 8.19 (s, 1H), 8.02 (dt, J=8.2, 1.2 Hz, 1H), 7.99-7.92 (m, 1H), 7.90 (d, J=0.7 Hz, 1H), 7.43 (ddd, J=7.3, 4.9, 1.4 Hz, 1H), 7.27 (d, J=3.6 Hz, 1H), 6.76 (d, J=3.6 Hz, 1H), 4.78 (s, 1H), 4.11 (s, 2H), 3.95 (s, 3H), 1.12 (s, 6H). m/z=407 (M+H)⁺.

### Preparation of V-3: N-(1-(2-hydroxy-2-methylpropyl)-3-(pyridin-2-yl)-1H-pyrazol-4-yl)-5-(1H-pyrazol-4-yl)furan-2-carboxamide.

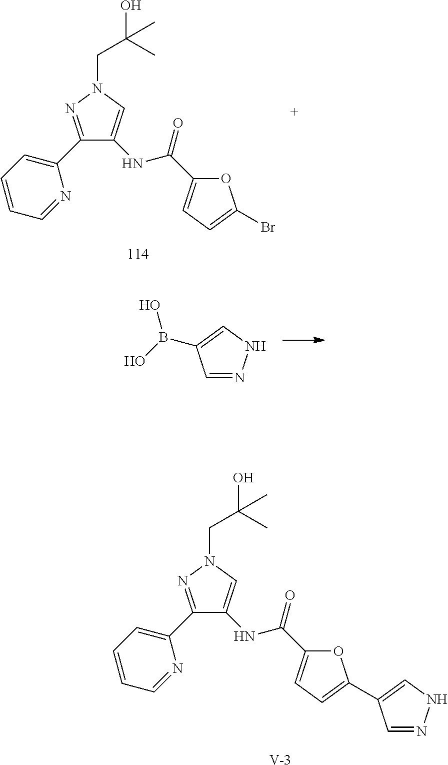

bromo-N-(1-(2-hydroxy-2-methylpropyl)-3-(pyridin-2-yl)-1H-pyrazol-4-yl)furan-2-carboxamide 114 (0.289 g, 0.71 mmol) was weighed out and added to a microwave reaction tube with magnetic stir bar. Pyrazole-4-boronic acid (0.511 g, 4.6 mmol) was added followed by 10 mL of a 7:3 dimethoxyethane/ethanol solution. Sodium carbonate (0.514 g, 4.8 mmol) was dissolved in 2.42 mL water and added to the reaction. This was subjected to vigorous sub-surface nitrogen sparge. Pd[P(Ph)₃]₂Cl₂(60 mg, 0.09 mmol) was added, the tube was sealed under nitrogen and then heated 30 minutes in the microwave at 130° C.

The solution was diluted into ethyl acetate and washed first with brine, then 1M aqueous sodium hydroxide, and again with brine before drying over sodium sulfate. (The base wash was analyzed for desired product to monitor potential loss to the aqueous layer.) Product solution was filtered, concentrated onto silica and purified by column chromatography. 0.180 g of the title compound V-3 was obtained after drying (64% yield).

¹H NMR (300 MHz, DMSO-d₆) δ 13.27 (s, 1H), 11.67 (s, 1H), 8.74 (ddd, J=5.0, 1.8, 0.9 Hz, 1H), 8.38 (s, 1H), 8.26 (s, 1H), 8.10-7.80 (m, 3H), 7.43 (ddd, J=7.3, 5.0, 1.4 Hz, 1H), 7.27 (d, J=3.5 Hz, 1H), 6.78 (d, J=3.5 Hz, 1H), 4.78 (s, 1H), 4.11 (s, 2H), 1.13 (s, 6H). m/z=393 (M+H)⁺.

### Preparation of V-4: tert-butyl 4-(54(1-(2-ethoxyethyl)-3-(pyridin-2-yl)-1H-pyrazol-4-yl)carbamoyl)furan-2-yl)-1H-pyrazole-1-carboxylate.

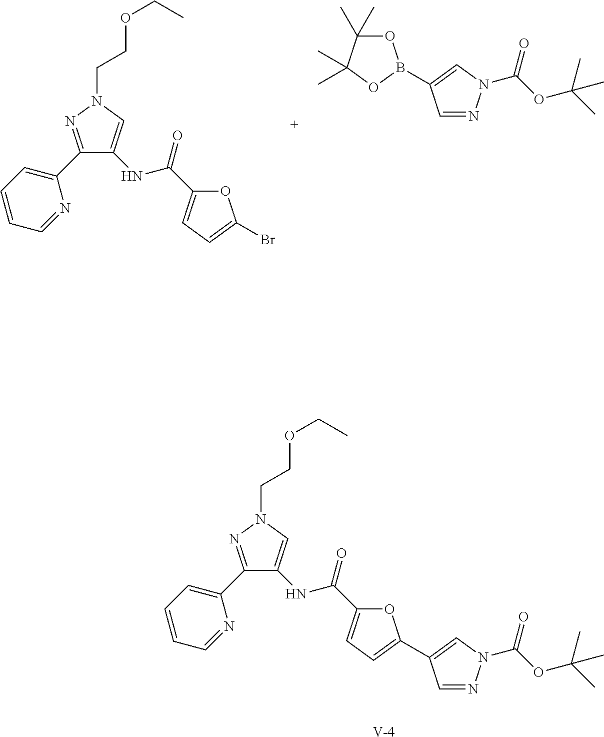

5-bromo-N-(1-(2-ethoxyethyl)-3-(pyridin-2-yl)-1H-pyrazol-4-yl)furan-2-carboxamide (2.435 g, 6.0 mmol) was weighed out and added to a reaction tube with magnetic stir bar. 1-Boc-pyrazole-4-boronic acid pinacol ester (3.535 g, 12.0 mmol) was added and these were dissolved in 60 mL dimethylformamide. Cesium carbonate (3.916 g, 12.0 mmol) was weighed out and added and the reaction was subjected to vigorous sub-surface nitrogen sparge. Pd(dppf)Cl₂.—CH₂Cl₂ (0.491 g, 0.60 mmol) was added followed by Ag₂O (1.391 g, 6.0 mmol). The tube was sealed under nitrogen and stirred overnight at room temperature. The reaction solution was then combined with a 0.64 mmol pilot reaction run under the same conditions and filtered through Celite with ethyl acetate washings. The filtrate was concentrated to dryness and partitioned between ethyl acetate and water. The aqueous layer is extracted three times more with ethyl acetate and the combined organic layer is washed with brine and dried over sodium sulfate. Product solution is filtered, concentrated onto silica and purified by column chromatography. Pure fractions are combined, concentrated and dried on high vacuum to give 2.2 g of the title compound V-4 (69% yield total).

¹H NMR (300 MHz, Chloroform-d) δ 11.83 (s, 1H), 8.69 (ddd, J=5.0, 1.9, 1.0 Hz, 1H), 8.60-8.33 (m, 2H), 8.29-7.91 (m, 2H), 7.79 (ddd, J=8.1, 7.5, 1.7 Hz, 1H), 7.28-7.21 (m, 2H), 6.62 (d, J=3.6 Hz, 1H), 4.35 (t, J=5.6 Hz, 2H), 3.86 (t, J=5.6 Hz, 2H), 3.51 (q, J=7.0 Hz, 2H), 1.72 (s, 9H), 1.19 (t, J=7.0 Hz, 3H). m/z=493 (M+H)⁺.

### Preparation of 2-bromo-N-(1-(2-hydroxy-2-methylpropyl)-3-(pyridin-2-yl)-1H-pyrazol-4-yl)thiazole-4-carboxamide 116.

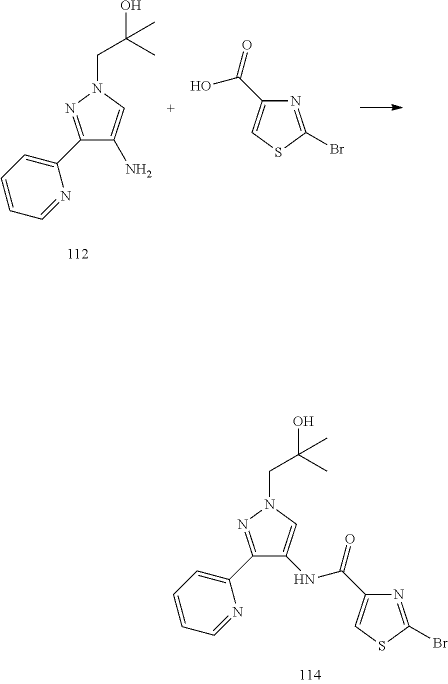 [SMILES: CC(C)(O)Cn1cc(N)c(-c2ccccn2)n1 ; O=C(O)c1csc(Br)n1 ; CC(C)(O)Cn1cc(NC(=O)c2csc(Br)n2)c(-c2ccccn2)n1]

2-Bromothiazole-4-carboxylic acid (0.257 g, 1.2 mmol) was weighed out and added to a flask with a magnetic stir bar and taken up in 53 mL dichloromethane. Diisopropylethylamine (0.322 mL, 1.8 mmol) was added followed by HATU (0.611 g, 1.6 mmol) and the reaction was stirred at room temperature for 60 minutes. 1-(4-Amino-3-(pyridin-2-yl)-1H-pyrazol-1-yl)-2-methylpropan-2-ol 112 (0.344 g, 1.5 mmol) was added in 21 mL dichloromethane solution and the reaction was stirred overnight at room temperature. This was concentrated directly onto silica and purified by column chromatography. Product containing fractions were all found to contain hydroxyazabenzotriazole as a contaminant. These were concentrated and partitioned between ethyl acetate and saturated aqueous sodium bicarbonate. The aqueous layer was washed with ethyl acetate until product was completely extracted. The combined organic layer was washed with brine and dried over sodium sulfate. Filtration, concentration and drying on high vacuum afforded 0.429 g of the pure title compound 114 (82% yield).

¹H NMR (300 MHz, DMSO-d₆) δ 12.23 (s, 1H), 8.70-8.57 (m, 1H), 8.42 (d, J=5.7 Hz, 2H), 8.06-7.87 (m, 2H), 7.39 (ddd, J=7.3, 4.9, 1.5 Hz, 1H), 4.78 (s, 1H), 4.12 (s, 2H), 1.12 (s, 6H). m/z=422/424 (M+H)⁺ (bromine isotopes).

### Preparation of VI-1: N-(1-(2-hydroxy-2-methylpropyl)-3-(pyridin-2-yl)-1H-pyrazol-4-yl)-2-(1H-pyrazol-4-yl)thiazole-4-carboxamide.

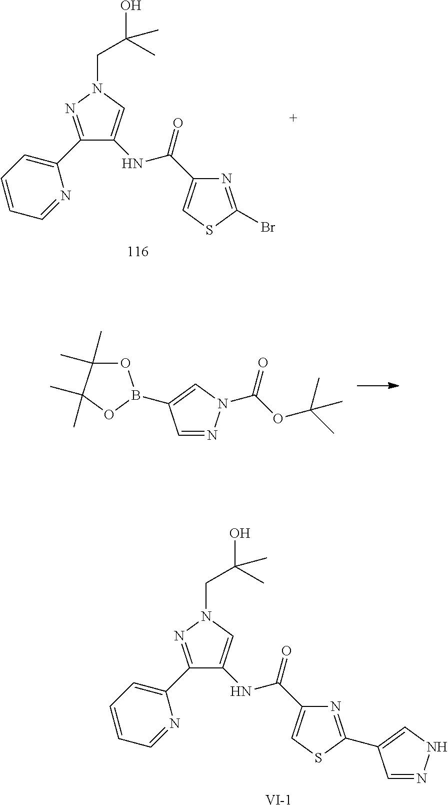 [SMILES: CC(C)(O)Cn1cc(NC(=O)c2csc(Br)n2)c(-c2ccccn2)n1 ; CC(C)(C)OC(=O)n1cc(B2OC(C)(C)C(C)(C)O2)cn1 ; CC(C)(O)Cn1cc(NC(=O)c2csc(-c3cn[nH]c3)n2)c(-c2ccccn2)n1 ; "VI-1"]

2-Bromo-N-(1-(2-hydroxy-2-methylpropyl)-3-(pyridin-2-yl)-1H-pyrazol-4-yl)thiazole -4-carboxamide 116 (0.212 g, 0.50 mmol) was weighed out and added to a microwave reaction vial with magnetic stir bar. 1-Boc-pyrazole-4-boronic acid pinacol ester (0.944 g, 3.2 mmol) was added followed by 4.9 mL dimethoxyethane and 2.1 mL ethanol. Sodium carbonate (0.362 g, 3.4 mmol) was dissolved in 1.7 mL water and added to the reaction. The solution was subjected to vigorous sub-surface nitrogen sparge and Pd[P(Ph)₃]₂Cl₂(60 mg, 0.09 mmol) was added. The tube was sealed under nitrogen and heated 30 minutes in the microwave at 130° C.

The solution was diluted into ethyl acetate and washed with saturated aqueous sodium bicarbonate and brine. The emulsified layer was back-extracted three times with ethyl acetate and the combined organic layer was dried over sodium sulfate. This was filtered, concentrated and purified by column chromatography to give 0.160 g of the title compound VI-1 after drying (78% yield).

¹H NMR (300 MHz, DMSO-d₆) δ 13.42 (s, 1H), 12.21 (s, 1H), 8.77 (ddd, J=5.0, 1.8, 1.0 Hz, 1H), 8.45 (s, 1H), 8.44-8.05 (br s, 2H), 8.28 (s, 1H), 8.03-7.90 (m, 2H), 7.42 (ddd, J=7.4, 4.9, 1.4 Hz, 1H), 4.79 (s, 1H), 4.12 (s, 2H), 1.13 (s, 6H). m/z=410 (M+H)⁺.

### Preparation of VI-11: N-(1-((1,3-cis)-3-ethoxycyclobutyl)-3-(pyridin-2-yl)-1H-pyrazol-4-yl)-2-(1H-pyrazol-4-yl)thiazole-4-carboxamide.

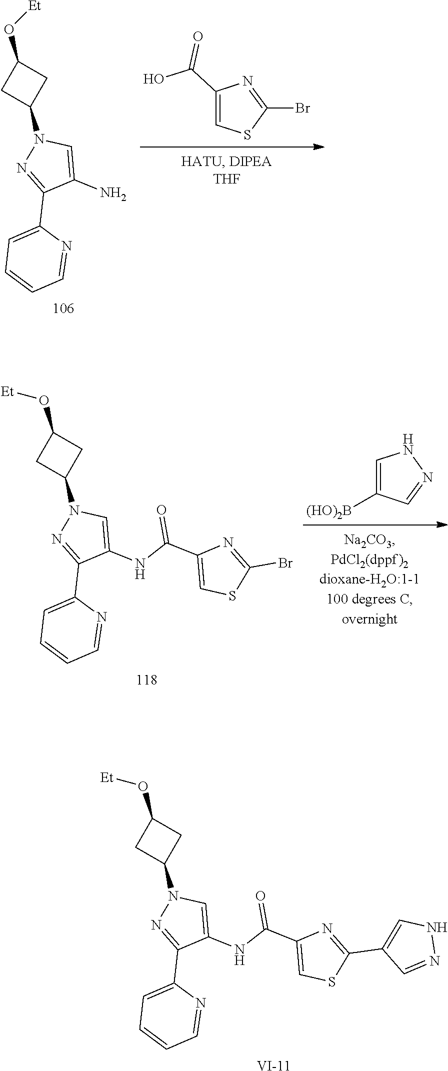

Compound 106 (680 mg), 2-bromothiazole-4-carboxylic acid (658 mg, 1.2 eq.), and HATU (1.5 g, 1.5 eq.) were dissolved in THF (30 mL) and DIPEA (0.7 mL, 1.5 eq.) was added to the solution. The reaction mixture was stirred at room temperature overnight and evaporated. The residue was purified by combiflash chromatography (EtOAc in hexanes=10-100%) to give product 118 (980 mg, 83% yield).

Compound 118 (1g), pyrazole-4-boronic acid (750 mg, 3 eq.), Na₂CO₃ (2.37 g, 10 eq.) and PdCl₂(dppf)₂ (200 mg) were stirred in dioxane (15 mL) and water (15 mL). The reaction mixture was heated at 100° C. overnight. LC-MS indicated fully conversion to the product. The reaction mixture was evaporated and purified by combiflash chromatography (2.0 M NH3/MeOH in DCM=0-20%) to give desired product VI-11 (700 mg, 72% yield). ¹H NMR (300 MHz, DMSO) δ 13.41 (br, 1H), 12.18 (s, 1H), 8.75 (d, J=4.5 Hz, 1H), 8.46 (m, 2H), 8.27 (s, 1H), 8.06 (m, 2H), 7.93 (m, 1H), 7.42 (m, 1H), 4.61 (p, J=8.1 Hz, 1H), 3.84 (p, J=6.9 Hz, 1H), 3.41 (q, J=6.9 Hz, 2H), 2.80 (m, 2H), 2.44 (m, 2H), 1.13 (t, J=6.9 Hz, 3H); LCMS: purity: 100%; MS (m/e): 436.56 (MH+).

### Preparation of 4-nitro-3-(trifluoromethyl)-1H-pyrazole 120.

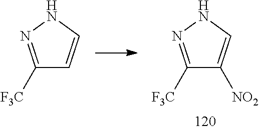 [SMILES: FC(F)(F)c1cc[nH]n1 ; O=[N+]([O-])c1c[nH]nc1C(F)(F)F]

72 mL concentrated sulfuric acid was added to a flask with magnetic stir bar and cooled to 0° C. 3-(trifluoromethyl)-pyrazole (12.070 g, 88.70 mmol) was weighed out and added gradually. An addition funnel was attached and charged with 90% fuming nitric acid (36 mL, 766 mmol). This was added in dropwise at 0° C., and the reaction was stirred warming to room temperature overnight. The reaction was then recharged with the same nitric acid described above (19 mL, 404 mmol) at room temperature and then stoppered. Stirring at room temperature continued overnight.

The reaction was poured over ice and neutalized by slow addition of 200 g sodium carbonate. The pH was adjusted to 6 with 1M hydrochloric acid and the solution was extracted six times with ethyl acetate. The combined organic layer was dried over sodium sulfate, filtered, and concentrated to an oil. This crystallized, and the solid was washed with minimal dichloromethane to give 3.250 g of the title compound 120 after drying. A second crop was isolated from the filtrate to give 1.752 g more product (31% yield). Additional product remained in the filtrate.

¹H NMR (300 MHz, DMSO-d₆) δ 9.16 (s, 1H). m/z=180 (M−H)⁻.

### Preparation of 3-(4-nitro-3-(trifluoromethyl)-1H-pyrazol-1-yl)cyclobutan-1-one 122.

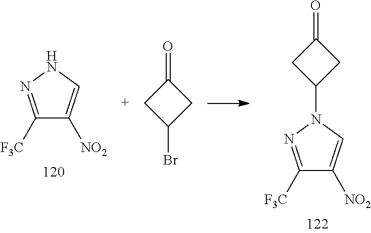 [SMILES: O=[N+]([O-])c1c[nH]nc1C(F)(F)F ; O=C1CC(Br)C1 ; O=C1CC(n2cc([N+](=O)[O-])c(C(F)(F)F)n2)C1]

Compound 120 (1.2356 g, 6.82 mmol) was dried in the tared reaction flask and weighed. This was taken up in 22 mL tetrahydrofuran, and a magnetic stir bar was added. 3-Bromocyclobutan-1-one (1.3837 g, 9.29 mmol) was weighed into a tared vial and added to the reaction in 11 mL tetrahydrofuran solution. Potassium carbonate (1.417 g, 10.25 mmol) was weighed out and added, and the reaction was stirred overnight at room temperature.

The reaction was next recharged with 3-bromocyclobutan-1-one (1.232 g, 8.27 mmol) in 5 mL tetrahydrofuran and stirred overnight at room temperature. The mixture was then concentrated to remove THF, and partitioned between ethyl acetate and water. The aqueous was extracted three times more with ethyl acetate and the combined organic layer was washed with brine and dried over sodium sulfate. This was filtered and concentrated and it spontaneously crystallized. The solid was collected, washed with a minimal volume of dichloromethane and dried on high vacuum to give 677.2 mg of the title compound 122. A second crop isolated after crystallizing from the filtrate gave 432.2 mg more product 122 (65% yield). A 1D NOE experiment confirmed the N1 assignment of the pyrazole alkylation.

¹H NMR (300 MHz, DMSO-d₆) δ 9.44 (s, 1H), 5.34 (p, J=6.9 Hz, 1H), 3.67 (d, J=6.7 Hz, 4H). Parent ion not observed.

### Preparation of (1s,3s)-3-(4-nitro-3-(trifluoromethyl)-1H-pyrazol-1-yl)cyclobutan-1-ol 124.

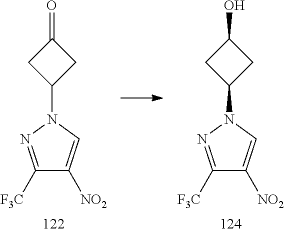 [SMILES: O=C1CC(n2cc([N+](=O)[O-])c(C(F)(F)F)n2)C1 ; O=[N+]([O-])c1cn([C@H]2C[C@@H](O)C2)nc1C(F)(F)F]

Compound 122 (601.0 mg, 2.41 mmol) was dried in the tared reaction flask and weighed. This was dissolved in 12 mL methanol, a magnetic stir bar was added, and the solution was cooled to 0° C. Sodium borohydride (137.9 mg, 3.64 mmol) was weighed out and added. The reaction was stirred 2 hours at room temperature. After HPLC showed completion, this was concentrated onto silica and purified by column chromatography. After drying, 536.2 mg was obtained of the title compound 124 (88% yield).

¹H NMR (300 MHz, DMSO-d₆) δ 9.23 (s, 1H), 5.38 (d, J=6.7 Hz, 1H), 4.63-4.46 (m, 1H), 4.06-3.89 (m, 1H), 2.83-2.70 (m, 2H), 2.42-2.29 (m, 2H). m/z=252 (M+H)⁺.

### Preparation of 1-((1s,3s)-3-ethoxycyclobutyl)-4-nitro-3-(trifluoromethyl)-1H-pyrazole 126.

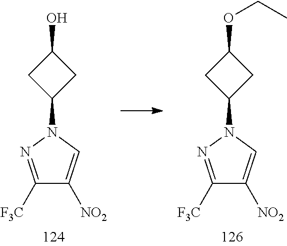 [SMILES: O=[N+]([O-])c1cn([C@H]2C[C@@H](O)C2)nc1C(F)(F)F ; CCO[C@H]1C[C@@H](n2cc([N+](=O)[O-])c(C(F)(F)F)n2)C1]

Compound 124 (189.6 mg, 0.76 mmol) was transferred to a reaction tube with magnetic stir bar in 5 mL dichloromethane. Silver triflate (586.2 mg, 2.28 mmol) was weighed out and added, and 2,6-di-t-butylpyridine was added (0.58 mL, 2.62 mmol). The reaction was cooled to 0° C. and ethyl iodide was added (0.20 mL, 2.50 mmol). The cooling bath was then removed, and it was stirred overnight at room temperature. This reaction was combined with another (46.0 mg, 0.18 mmol) run under the same conditions and filtered through Celite with dichloromethane washings. The filtrate was concentrated onto silica and purified by column chromatography. After drying, 172.8 mg was obtained of the pure title compound 126 (66% yield).

¹H NMR (300 MHz, Chloroform-d) δ 8.33 (s, 1H), 4.46 (tt, J=9.0, 7.5 Hz, 1H), 3.90 (tt, J=7.5, 6.4 Hz, 1H), 3.47 (q, J=7.0 Hz, 2H), 3.03-2.91 (m, 2H), 2.57-2.44 (m, 2H), 1.23 (t, J=7.0 Hz, 3H). m/z=280 (M+H)⁺.

### Preparation of 1-((1s,3s)-3-ethoxycyclobutyl)-3-(trifluoromethyl)-1H-pyrazol-4-amine 128.

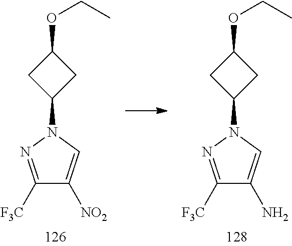 [SMILES: CCO[C@H]1C[C@@H](n2cc([N+](=O)[O-])c(C(F)(F)F)n2)C1 ; CCO[C@H]1C[C@@H](n2cc(N)c(C(F)(F)F)n2)C1]

Compound 126 (231.4 mg, 0.83 mmol) was added to a Parr reaction bottle in 30 mL ethyl acetate. This was put under nitrogen and charged with (wet) 10% Pd on carbon (90.1 mg, 0.04 mmol). This was run at 50 psi hydrogen for 5 hours on the Parr hydrogenator. The reaction was filtered through Celite with methanol washings and concentrated to dryness. HPLC showed a complex mixture. 110.6 mg of this residue was dissolved in 10 mL methanol. NiCl₂.×hydrate (400.1 mg, 1.68 mmol as the hexahydrate) was weighed out and added, and the mixture was cooled to 0° C. Sodium borohydride (127.4 mg, 3.4 mmol) was weighed out and added slowly, portionwise. The reaction was allowed to stir overnight, warming to room temperature. This was filtered through Celite with methanol washings, concentrated onto silica and purified by column chromatography. After drying, 76.2 mg was obtained of the title compound as an oil. (The remainder of the residue recovered from the hydrogenation was reduced using similar conditions and an additional 46.1 mg of the title compound 128 was obtained-59% yield).

¹H NMR (300 MHz, Chloroform-d) δ 7.17 (s, 1H), 4.31 (tt, J=9.1, 7.5 Hz, 1H), 3.82 (tt, J=7.6, 6.5 Hz, 1H), 3.44 (q, J=7.0 Hz, 2H), 2.93-2.80 (m, 2H), 2.45-2.32 (m, 2H), 1.22 (t, J=7.0 Hz, 3H). m/z=250 (M+H)⁺.

### Preparation of 2-bromo—N-(1-((1s,3s)-3-ethoxycyclobutyl)-3-(trifluoromethyl)-1H-pyrazol-4-yl)thiazole-4-carboxamide 130.

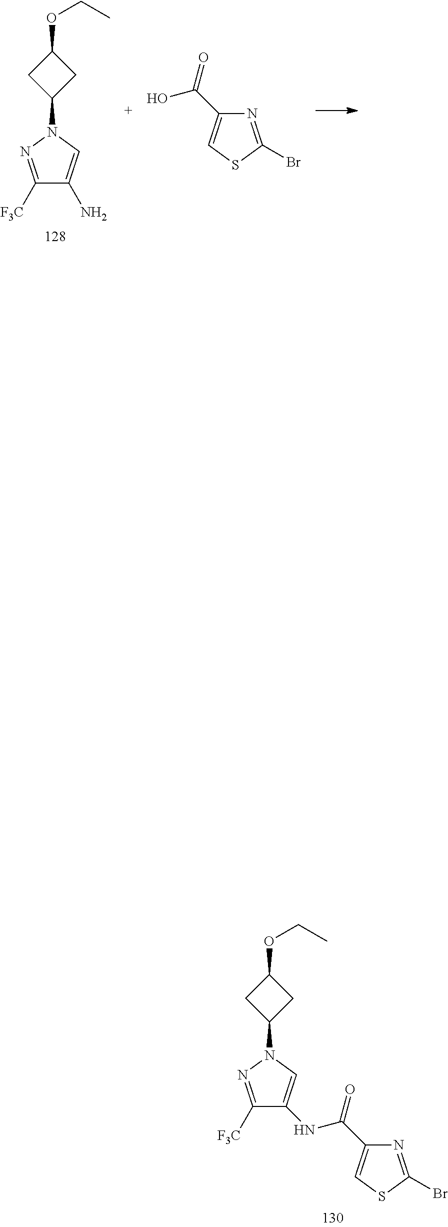 [SMILES: CCO[C@H]1C[C@@H](n2cc(N)c(C(F)(F)F)n2)C1 ; O=C(O)c1csc(Br)n1 ; CCO[C@H]1C[C@@H](n2cc(NC(=O)c3csc(Br)n3)c(C(F)(F)F)n2)C1]

2-Bromothiazole-4-carboxylic acid (61.4 mg, 0.30 mmol) was weighed out and added to a flask with a magnetic stir bar and taken up in 12 mL dichloromethane. Diisopropylethylamine (0.077 mL, 0.44 mmol) was added followed by HATU (145.4 mg, 0.38 mmol) and the reaction was stirred at room temperature for 45 minutes. Compound 128 (73 mg, 0.29 mmol) was added in 5 mL dichloromethane solution and the reaction was stirred overnight at room temperature. This was concentrated directly onto silica and purified by column chromatography. Concentrating, then drying the pure fractions on high vacuum afforded 71.0 mg of the title compound 130 (55% yield).

¹H NMR (300 MHz, Chloroform-d) δ 9.12 (s, 1H), 8.40 (s, 1H), 8.13 (s, 1H), 4.52-4.32 (m, 1H), 3.86 (tt, J=7.6, 6.5 Hz, 1H), 3.46 (q, J=7.0 Hz, 2H), 2.91 (dddd, J=9.3, 7.5, 6.5, 2.9 Hz, 2H), 2.52 (qdd, J=9.9, 5.2, 2.6 Hz, 2H), 1.23 (t, J=7.0 Hz, 3H). m/z=439/441 (M+H)⁺ (bromine isotopes).

### Preparation of VI-62: N-(1-((1s,3s)-3-ethoxycyclobutyl)-3-(trifluoromethyl)-1H-pyrazol-4-yl)-2-(1H-pyrazol-4-yl)thiazole-4-carboxamide.

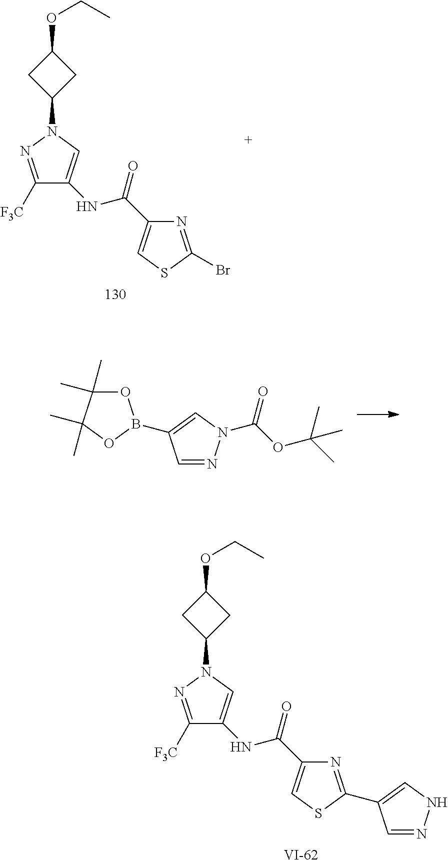 [SMILES: CCO[C@H]1C[C@@H](n2cc(NC(=O)c3csc(Br)n3)c(C(F)(F)F)n2)C1 ; CC(C)(C)OC(=O)n1cc(B2OC(C)(C)C(C)(C)O2)cn1 ; CCO[C@H]1C[C@@H](n2cc(NC(=O)c3csc(-c4cn[nH]c4)n3)c(C(F)(F)F)n2)C1 ; "VI-62"]

Compound 130 (67.7 mg, 0.15 mmol) was transferred to a microwave reaction tube with magnetic stir bar in solution (4.2 mL dimethoxyethane and 3.0 mL ethanol). 1-Boc-pyrazole-4-boronic acid pinacol ester (290.6 mg, 1.0 mmol) was weighed out and added. Sodium carbonate (109.0 mg, 1.0 mmol) was weighed into a tared vial, dissolved in 1.0 mL water, and added to the reaction. The solution was subjected to vigorous sub-surface nitrogen sparge. Pd[P(Ph)₃]₂Cl₂(18.4 mg, 0.03 mmol) was weighed out and added and the tube was sealed under nitrogen. This was heated 30 minutes at 100° C. in the microwave. The solution was partitioned between ethyl acetate and saturated aqueous sodium bicarbonate. The aqueous layer was extracted three more times with ethyl acetate and the combined organic layer was washed with brine and dried over sodium sulfate. This was filtered, concentrated and subjected to column chromatography. The purest fractions were concentrated to give a solid which was triturated with acetonitrile and dried on high vacuum to give 8.0 mg of the title compound VI-62. (Additional less pure material was recovered.)

¹H NMR (300 MHz, Chloroform-d) δ 9.44 (s, 1H), 8.45 (s, 1H), 8.12 (s, 2H), 8.08 (s, 1H), 4.43 (ddd, J=16.6, 9.3, 7.5 Hz, 1H), 3.87 (tt, J=7.7, 6.4 Hz, 1H), 3.47 (q, J=7.0 Hz, 2H), 2.92 (dddd, J=9.3, 7.5, 6.5, 3.3 Hz, 2H), 2.54 (tdd, J=9.3, 7.7, 2.9 Hz, 2H), 1.23 (t, J=7.0 Hz, 3H). m/z=427 (M+H)⁺.

### Preparation of 2-bromo—N-(1-methyl-3-(trifluoromethyl)-1H-pyrazol-4-yl)thiazole-4-carboxamide 132.

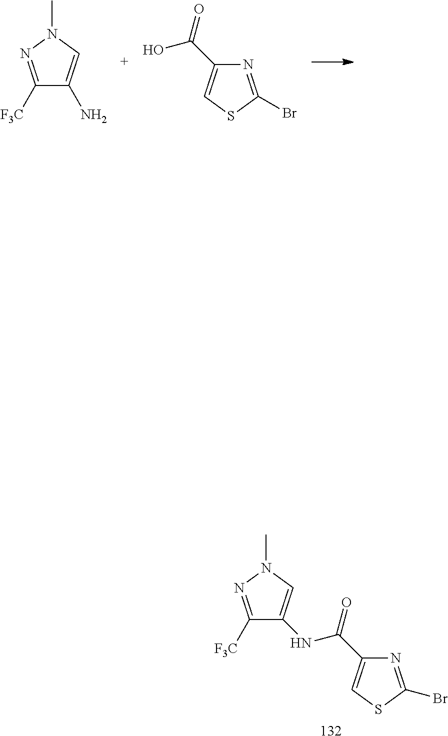 [SMILES: Cn1cc(N)c(C(F)(F)F)n1 ; O=C(O)c1csc(Br)n1 ; Cn1cc(NC(=O)c2csc(Br)n2)c(C(F)(F)F)n1 ; "132"]

Bromothiazole-4-carboxylic acid (416.2 mg, 2.00 mmol) was weighed out and added to a flask with a magnetic stir bar and taken up in 40 mL dichloromethane. Diisopropylethylamine (0.52 mL, 3.0 mmol) was added followed by HATU (990.4 mg, 2.60 mmol) and the reaction was stirred at room temperature for 45 minutes. 1-methyl-3-(trifluoromethyl)-1H-pyrazol-4-amine (329.4 mg, 2.00 mmol) was added in 10 mL dichloromethane solution and the reaction was stirred overnight at room temperature. This was concentrated directly onto silica and purified by column chromatography. After drying, 471.6 mg was obtained of the title compound 132 (66% yield- additional less pure material was recovered).

¹H NMR (300 MHz, Chloroform-d) δ 9.12 (s, 1H), 8.29 (s, 1H), 8.13 (s, 1H), 3.96 (s, 3H). m/z=355/357 (M+H)⁺ (bromine isotopes).

### Preparation of VI-63: N-(1-methyl-3-(trifluoromethyl)-1H-pyrazol-4-yl)-2-(1H-pyrazol-4-yl)thiazole-4-carboxamide trifluoroacetate salt.

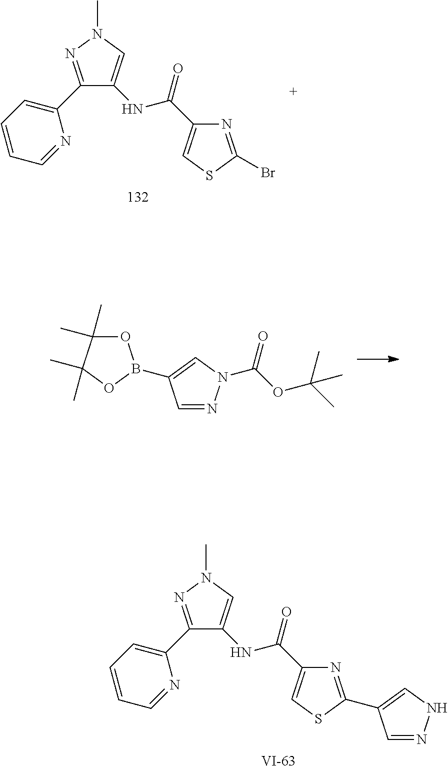 [SMILES: Cn1cc(NC(=O)c2csc(Br)n2)c(-c2ccccn2)n1 ; CC(C)(C)OC(=O)n1cc(B2OC(C)(C)C(C)(C)O2)cn1 ; Cn1cc(NC(=O)c2csc(-c3cn[nH]c3)n2)c(-c2ccccn2)n1 ; "VI-63"]

Compound 132 (100.0 mg, 0.28 mmol) and 1-Boc-pyrazole-4-boronic acid pinacol ester (531.4 mg, 1.80 mmol) were weighed out and added to a microwave reaction tube with magnetic stir bar. 7.7 mL dimethoxyethane and 5.5 mL ethanol were added. Sodium carbonate (200.2 mg, 1.89 mmol) was weighed into a tared vial, dissolved in 2.0 mL water, and added to the reaction. The solution was subjected to vigorous sub-surface nitrogen sparge. Pd[P(Ph)₃]₂Cl₂(34.4 mg, 0.05 mmol) was weighed out and added and the tube was sealed under nitrogen. This was heated 30 minutes at 100° C. in the microwave. This was concentrated to remove dimethoxyethane and ethanol and extracted four times with ethyl acetate. The combined organic layer was washed with brine, dried over sodium sulfate, filtered and concentrated. This was purified by preparative HPLC to give compound VI-64. After drying, 54.3 mg was obtained of the title compound VI-63 as a trifluoroacetic acid salt.

¹H NMR (300 MHz, DMSO-d₆) δ 9.61 (s, 1H), 8.32 (s, 1H), 8.25 (s, 2H), 3.95 (s, 3H). m/z=343 (M+H)⁺.

### Preparation of (1s,3s)-3-(4-amino-3-(3-fluoropyridin-2-yl)-1H-pyrazol-1-yl)cyclobutan-1-ol 134.

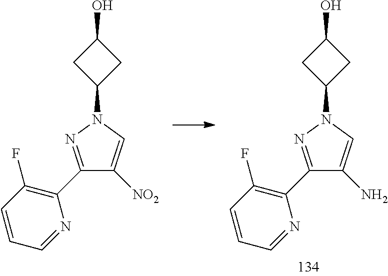 [SMILES: O=[N+]([O-])c1cn([C@H]2C[C@@H](O)C2)nc1-c1ncccc1F ; Nc1cn([C@H]2C[C@@H](O)C2)nc1-c1ncccc1F]

(1s,3s)-3-(3-(3-fluoropyridin-2-yl)-4-nitro-1H-pyrazol-1-yl)cyclobutan-1-ol (1.070 g, 3.85 mmol) was weighed out and added to a flask with magnetic stir bar, and dissolved in 98 mL ethyl acetate. This was put under nitrogen and charged with (wet) 10% Pd on carbon (117.8 mg, 0.014 mmol). After thoroughly purging with nitrogen, this was stirred for 3 hours under a balloon of hydrogen. The reaction was then filtered through Celite with excess ethyl acetate washings. The filtrate was concentrated and dried to give quantitative recovery of the title compound 134 as a foam. This was used in the next reaction without further purification.

¹H NMR (300 MHz, DMSO-d₆) δ 8.47-8.31 (m, 1H), 7.79-7.62 (m, 1H), 7.35-7.22 (m, 2H), 5.26 (d, J=6.6 Hz, 1H), 4.94 (s, 2H), 4.34-4.18 (m, 1H), 3.93 (td, J=7.4, 6.0 Hz, 1H), 2.71 (dtd, J=8.7, 7.1, 3.0 Hz, 2H), 2.27 (qd, J=8.7, 2.9 Hz, 2H). m/z=249 (M+H)⁺.

### Preparation of 2-bromo—N-(3-(3-fluoropyridin-2-yl)-1-((1s,3s)-3-hydroxycyclobutyl)-1H-pyrazol-4-yl)thiazole-4-carboxamide 136.

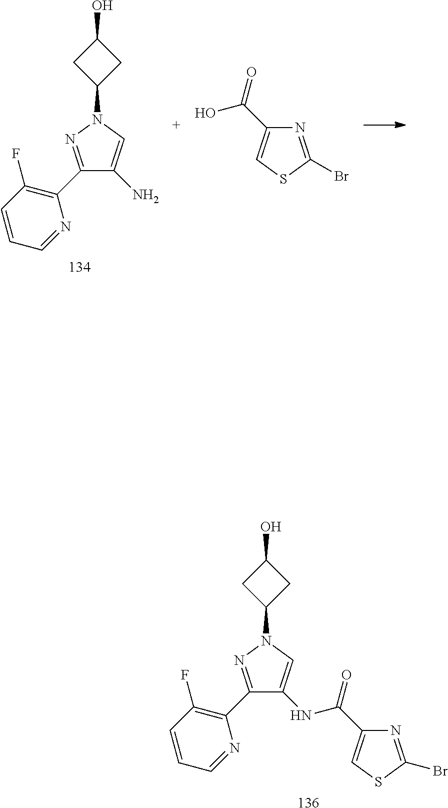 [SMILES: Nc1cn([C@H]2C[C@@H](O)C2)nc1-c1ncccc1F ; O=C(O)c1csc(Br)n1 ; O=C(Nc1cn([C@H]2C[C@@H](O)C2)nc1-c1ncccc1F)c1csc(Br)n1 ; "136"]

Compound 134 (0.96 g, 3.85 mmol) was dried in the tared reaction flask and weighed. This was dissolved in 30 mL dichloromethane, and 10 mL dimethylformamide was added along with a magnetic stir bar.

2-Bromothiazole-4-carboxylic acid (800.6 mg, 3.85 mmol) was weighed out and added. Diisopropylethylamine (1.0 mL, 5.7 mmol) was added followed by HATU (1.901 g, 5.00 mmol) and the reaction was stirred at room temperature overnight. This was concentrated directly onto silica and purified by column chromatography. Concentrating, then drying the pure fractions on high vacuum afforded 1.158 g of the title compound 136 (69% yield).

¹H NMR (300 MHz, DMSO-d₆) δ 12.14 (s, 1H), 8.57-8.48 (m, 2H), 8.44 (s, 1H), 7.91 (ddd, J=11.5, 8.4, 1.3 Hz, 1H), 7.52 (ddd, J=8.4, 4.6, 3.8 Hz, 1H), 5.34 (d, J=6.9 Hz, 1H), 4.52 (tt, J=9.1, 7.3 Hz, 1H), 4.05-3.91 (m, 1H), 2.86-2.72 (m, 2H), 2.39 (qd, J=8.6, 2.8 Hz, 2H). m/z=438/440 (M+H)⁺(bromine isotopes).

### Preparation of VI-65: N-(3-(3-fluoropyridin-2-yl)-1-((1s,3s)-3-hydroxycyclobutyl)-1H-pyrazol-4-yl)-2-(1H-pyrazol-4-yl)thiazole-4-carboxamide.

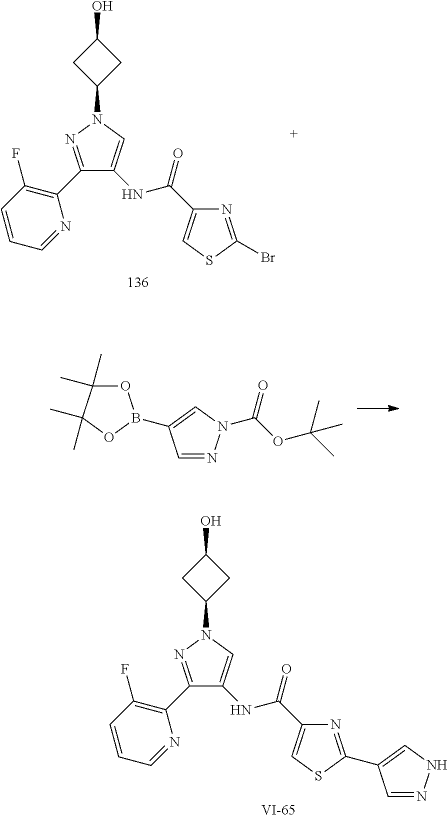 [SMILES: O=C(Nc1cn([C@H]2C[C@@H](O)C2)nc1-c1ncccc1F)c1csc(Br)n1 ; CC(C)(C)OC(=O)n1cc(B2OC(C)(C)C(C)(C)O2)cn1 ; O=C(Nc1cn([C@H]2C[C@@H](O)C2)nc1-c1ncccc1F)c1csc(-c2cn[nH]c2)n1 ; "VI-65"]

Compound 136 (0.497 g, 1.13 mmol) was transferred to a microwave reaction tube with magnetic stir bar in solution (13 mL dimethoxyethane and 5.5 mL ethanol). 1-Boc-pyrazole-4-boronic acid pinacol ester (1.334 g, 4.53 mmol) was weighed out and added. Sodium carbonate (0.480 g, 4.53 mmol) was weighed into a tared vial, dissolved in 4.5 mL water, and added to the reaction. The solution was subjected to vigorous sub-surface nitrogen sparge. Pd[P(Ph)₃]₂Cl₂(79.6 mg, 0.11 mmol) was weighed out and added and the tube was sealed under nitrogen. This was heated 90 minutes at 100° C. in the microwave. This was concentrated to remove dimethoxyethane and ethanol and extracted four times with ethyl acetate. However, there was substantial undissolved solid. This was collected and washed repeatedly with methanol. After drying, this gave 174.0 mg of the title compound at 90% purity.

The combined organic layer from the extraction was washed with brine, dried over sodium sulfate, filtered, and combined with the methanol washings of the precipitated solid. The solution was concentrated onto silica and purified by column chromatography. Concentration of pure fractions gave a solid which was triturated with minimal dichloromethane. After drying, 169.2 mg was obtained of the pure title compound VI-65.

¹H NMR (300 MHz, DMSO-d₆) δ 13.43 (s, 1H), 12.09 (s, 1H), 8.66 (dt, J=4.6, 1.4 Hz, 1H), 8.57 (s, 1H), 8.50 (s, 1H), 8.30 (s, 1H), 8.11 (s, 1H), 7.91 (ddd, J=11.5, 8.4, 1.3 Hz, 1H), 7.54 (ddd, J=8.4, 4.6, 3.8 Hz, 1H), 5.34 (d, J=6.9 Hz, 1H), 4.61-4.42 (m, 1H), 3.98 (h, J=7.4 Hz, 1H), 2.80 (dtd, J=9.6, 6.9, 2.8 Hz, 2H), 2.47-2.33 (m, 2H). m/z=426 (M+H)⁺.

### Preparation of 2-(4-nitro-1-(1,4-dioxaspiro[4.5]decan-8-yl)-1H-pyrazol-3-yl)pyridine 138.

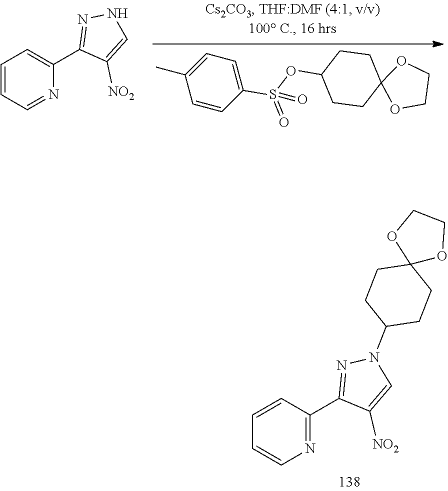

A stirring suspension of 2-(4-nitro-1H-pyrazol-3-yl)pyridine (950 mg, 5.00 mmol), 1,4-dioxaspiro[4.5]decan-8-yl 4-methylbenzenesulfonate (1.69 g, 5.41 mmol) and Cs₂CO₃ (2.44 g, 7.50 mmol) in anhydrous THF:DMF (15 mL, 4:1, v/v) was heated to 100° C. and stirred for 16 hours. The reaction mixture was diluted in water (50 mL), extracted with EtOAc (3×50 mL), the organic layer was washed with brine (50 mL), dried over MgSO₄, concentrated and column chromatography (0-100% EtOAc in hexane, gradient) gave compound 138 as a light brown semisolid (910 mg, 55.14%). MS (m/e): 330.34 (MH+).

### Preparation of 4-(4-nitro-3-(pyridin-2-yl)-1H-pyrazol-1-yl)cyclohexan-1-one 140.

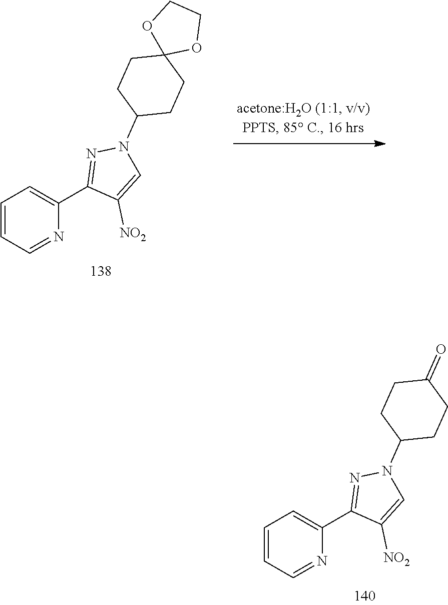 [SMILES: O=[N+]([O-])c1cn(C2CCC3(CC2)OCCO3)nc1-c1ccccn1 ; O=C1CCC(n2cc([N+](=O)[O-])c(-c3ccccn3)n2)CC1]

To a stirring solution of compound 138 (910 mg, 2.75 mmol) in acetone:H₂O (20 mL, 1:1, v/v) was added pyridinium p-tolulene sulfonate (1.38 g, 5.50 mmol) and the reaction mixture was heated to 80° C. and stirred for 16 hours. Acetone was evaporated in vacuo, the aqueous layer was quenched with NaOH to pH=8, extracted with EtOAc (3×50 mL), the organic layer was washed with brine (50 mL), dried over MgSO₄, concentrated and column chromatography (0-100% MeOH in DCM, gradient) gave compound 140 as a dark brown oil (600 mg, 76.08%). MS (m/e): 286.29 (MH+).

### Preparation of (trans)-4-(4-nitro-3-(pyridin-2-yl)-1H-pyrazol-1-yl)cyclohexan-1-ol 142.

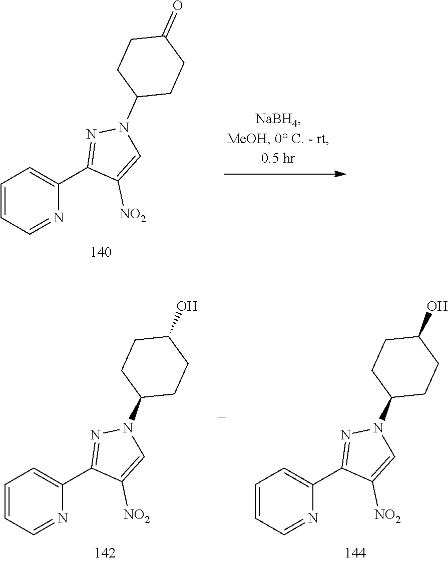 [SMILES: O=C1CCC(n2cc([N+](=O)[O-])c(-c3ccccn3)n2)CC1 ; O=[N+]([O-])c1cn([C@H]2CC[C@H](O)CC2)nc1-c1ccccn1 ; O=[N+]([O-])c1cn([C@H]2CC[C@@H](O)CC2)nc1-c1ccccn1]

NaBH₄ (20 mg, 0.524 mmol) was added to a stirring solution of 2 (600 mg, 2.10 mmol) in MeOH (10 mL) at 0° C., stirred for 0.5 hours, concentrated and column chromatography (0-100% MeOH (1M NH₃ solution) in DCM, gradient) afforded the product 142 as a viscous oil (362 mg, 60%).

¹H NMR (300 MHz, Chloroform-d) δ 8.77 (d, J=4.8 Hz, 1H), 8.29 (s, 1H), 7.84 (m, 2H), 7.36 (m, 1H), 4.24 (m, 1H), 3.76 (m, 1H), 3.46 (s, 1H), 2.14 (m, 8H). LCMS: purity: 87.43%. MS (m/e): 288.31 (MH+).

### Preparation of 2-(1-((trans)-4-ethoxycyclohexyl)-4-nitro-1H-pyrazol-3-yl)pyridine 146.

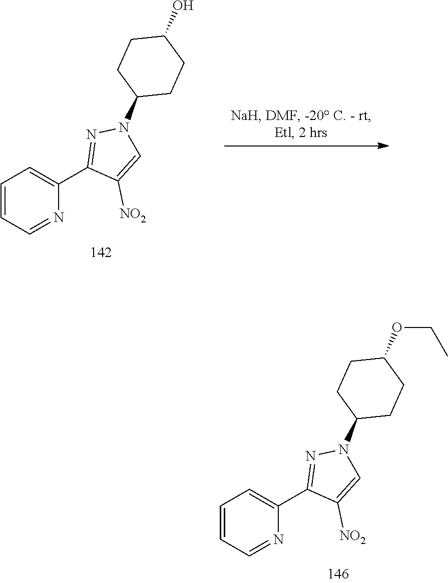 [SMILES: O=[N+]([O-])c1cn([C@H]2CC[C@H](O)CC2)nc1-c1ccccn1 ; CCO[C@H]1CC[C@H](n2cc([N+](=O)[O-])c(-c3ccccn3)n2)CC1]

NaH (60% dispersion in mineral oil, 60 mg, 1.50 mmol) was added to a stirring solution of compound 142 (360 mg, 1.25 mmol) and iodoethane (200-L, 2.50 mmol) in anhydrous DMF (8 mL) at −20° C. The reaction mixture was allowed to warm to room temperature for 2 hours. The reaction mixture was diluted in water (40 mL), extracted with EtOAc (3×50 mL), the organic layer was washed with brine (30 mL), dried over MgSO₄, concentrated, and column chromatography (0-100% EtOAc in hexane, gradient) afforded the product 146 as viscous oil (296 mg, 74.93%). MS (m/e): 316.36 (MH+).

### Preparation of 1-((trans)-4-ethoxycyclohexyl)-3-(pyridin-2-yl)-1H-pyrazol-4-amine 148.

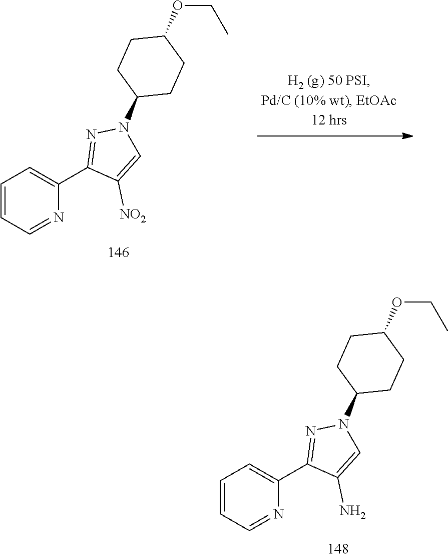 [SMILES: CCO[C@H]1CC[C@H](n2cc([N+](=O)[O-])c(-c3ccccn3)n2)CC1 ; CCO[C@H]1CC[C@H](n2cc(N)c(-c3ccccn3)n2)CC1]

A solution of compound 146 (290 g, 0.917 mmol) in EtOAc (10 mL) with Pd/C (10% wt, 50 mg) was hydrogenated under 50 psi H₂ (g) for 12 hours, filtered through celite and concentrated to give compound 148 as a viscous oil (230 mg, 87.61%). MS (m/e): 286.38 (MH+).

### Preparation of 2-bromo—N-(1-((trans)-4-ethoxycyclohexyl)-3-(pyridin-2-yl)-1H-pyrazol-4-yl)thiazole-4-carboxamide 150.

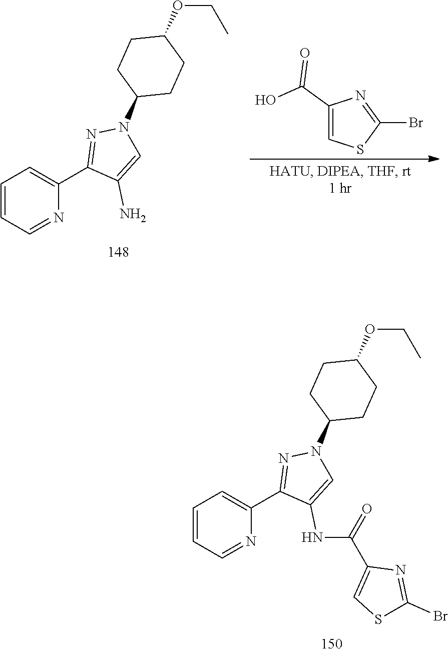 [SMILES: CCO[C@H]1CC[C@H](n2cc(N)c(-c3ccccn3)n2)CC1 ; O=C(O)c1csc(Br)n1 ; CCO[C@H]1CC[C@H](n2cc(NC(=O)c3csc(Br)n3)c(-c3ccccn3)n2)CC1]

HATU (458 mg, 1.20 mmol) was added to a stirring solution of 2-bromothiazole-4-carboxylic acid (184 mg, 0.883 mmol) and DIPEA (280-L, 1.61 mmol) in anhydrous THF (4 mL) at room temperature for 10 minutes, followed by addition of a solution of compound 148 (230 mg, 0.803 mmol) in anhydrous THF (4 mL). After 1 hour, the reaction mixture was diluted in water (10 mL), extracted with EtOAc (3×20 mL), the organic layer was washed with brine (20 mL), dried over MgSO₄, concentrated, and column chromatography (0-100% EtOAc in hexane, gradient) afforded the product 150 as a semisolid, which was used without further purification. Assumed quantitative yield. MS (m/e): 476.39 (MH+).

### Preparation of VI-145: N-(1-((trans)-4-ethoxycyclohexyl)-3-(pyridin-2-yl)-1H-pyrazol-4-yl)-2-(1H-pyrazol-4-yl)thiazole-4-carboxamide.

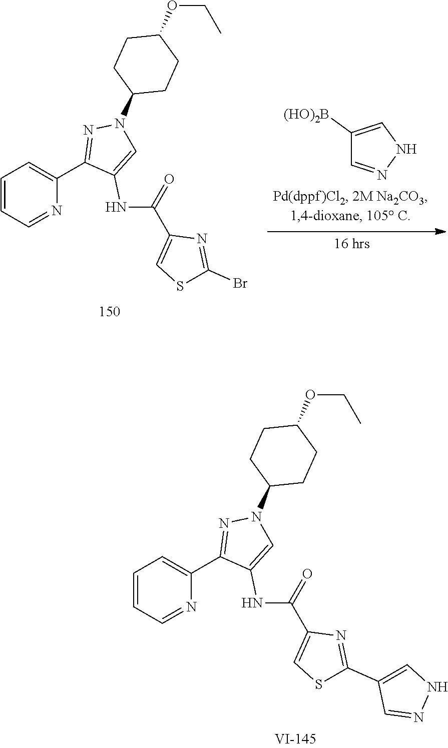 [SMILES: CCO[C@H]1CC[C@H](n2cc(NC(=O)c3csc(Br)n3)c(-c3ccccn3)n2)CC1 ; OB(O)c1cn[nH]c1 ; CCO[C@H]1CC[C@H](n2cc(NC(=O)c3csc(-c4cn[nH]c4)n3)c(-c3ccccn3)n2)CC1]

A mixture of crude compound 150 (0.803 mmol), 1H-pyrazole-4-boronic acid (180 mg, 1.61 mmol), Pd(dppf)Cl₂ (65.6 mg, 0.080 mmol), 2 M Na₂CO₃ (1.61 mL, 3.21 mmol) and anhydrous 1,4-dioxane (10 mL) was heated at 105° C. and stirred for 16 hours. The reaction mixture was cooled to room temperature, diluted in water (20 mL), extracted with EtOAc (3×30 mL), the organic layer was washed with brine (20 mL), dried over MgSO₄, concentrated, and column chromatography (0-100% EtOAc in hexane, gradient) gave a semisolid, which was submitted for analytical purification, followed by lyophilization to afford the title compound VI-145 as a white fluffy solid (75 mg, 20.15%).

¹H NMR (300 MHz, DMSO-d₆) δ 13.40 (s, 1H), 12.18 (s, 1H), 8.74 (d, J=4.8 Hz, 1H), 8.49 (s, 1H), 8.35 (s, 1H), 8.27 (s, 1H), 8.10 (s, 1H), 7.97 (m, 2H), 7.39 (t, J=6.9 Hz, 1H), 4.29 (t, J=11.7 Hz, 1H), 3.47 (td, J=7.1, 5.8 Hz, 2H), 3.35 (t, J=11.7 Hz, 1H), 2.09 (d, J=11.6 Hz, 4H), 1.87 (q, J=11.8 Hz, 2H), 1.35 (q, J=11.2 Hz, 2H), 1.10 (t, J=6.9 Hz , 3H). LCMS: purity: 100%. MS (m/e): 463.56 (MH+).

### VI-77: (4-(4-((1-((1,3-cis)-3-ethoxycyclobutyl)-3-(pyridin-2-yl)-1H-pyrazol-4-yl)carbamoyl)thiazol-2-yl)-1H-pyrazol-1-yl)methyl phosphate bis-potassium salt.

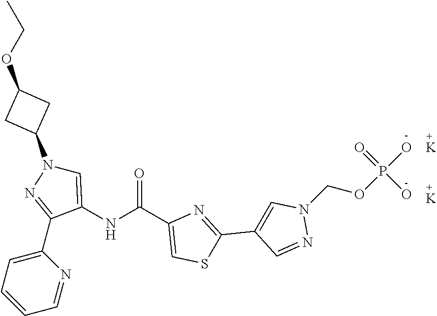

To a mixture of (4-(4-((1-((1,3-cis)-3-ethoxycyclobutyl)-3-(pyridin-2-yl)-1H-pyrazol-4-yl)carbamoyl)thiazol-2-yl)-1H-pyrazol-1-yl)methyl phosphate (300 mg) in acetonitrile (2 mL) and water (1 mL), was added 1.0 N potassium hydroxide aqueous solution (1.1 mL, 2 eq.) After sonicating for five minutes, the solution was lyophilized for 24 hours. The resulting powder was suspended in water (1 mL) and isopropanol (5 mL). The mixture was stirred at 70° C. for five minutes until a clear solution formed. The solution was cooled to room temperature. The resulting precipitate was collected through filtration, washed with isopropanol (3×1 mL) and dried under high vacuum at room temperature for 24 hours to give potassium salt as a white solid (280 mg).

¹H NMR (300 MHz, Deuterium Oxide) 6 7.83 (d, 1H), 7.80 (s, 1H), 7.64 (s, 1H), 7.42 (s, 1H), 7.41 (m, 1H), 7.29 (s, 1H), 7.17 (d, J=7.2 Hz, 1H), 6.89 (m, 1H), 5.57 (d, J=8.1 Hz, 2H), 4.13 (m, 1H), 3.91 (t, J=7.8 Hz, 1H), 3.49 (q, J=7.2 Hz, 2H), 2.83 (m, 2H), 2.19 (m, 2H), 1.14 (t, J=7.2 Hz, 3H); LCMS: purity: 100%; MS (m/e): 546.23 (MH+).

### VI-78: (4-(44(14(1,3-cis)-3-ethoxycyclobutyl)-3-(pyridin-2-yl)-1H-pyrazol-4-yl)carbamoyl)thiazol-2-yl)-1H-pyrazol-1-yl)methyl phosphate calcium salt.

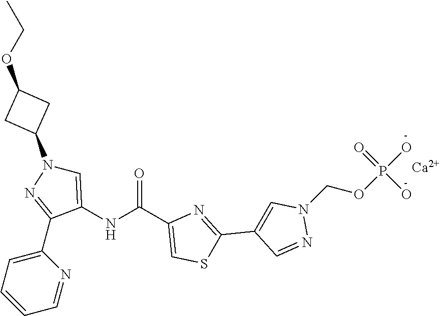 [SMILES: CCO[C@H]1C[C@@H](n2cc(NC(=O)c3csc(-c4cnn(COP(=O)(O)O)c4)n3)c(-c3ccccn3)n2)C1 ; [Ca+2]]

To a mixture of (4-(4-((1-((1,3-cis)-3-ethoxycyclobutyl)-3-(pyridin-2-yl)-1H-pyrazol-4-yl)carbamoyl)thiazol-2-yl)-1H-pyrazol-1-yl)methyl phosphate (309 mg) in acetonitrile (2 mL) and water (1 mL), was added calcium hydroxide (42 mg, 1 eq.). After sonicating for five minutes, the reaction mixture was lyophilized for 24 hours. The resulting powder was suspended in water (1 mL) and isopropanol (5 mL). The mixture was stirred at 70° C. for five minutes and then cooled to room temperature. The resulting precipitate was collected through filtration, washed with isopropanol (3×1 mL) and dried under high vacuum at room temperature for 24 hours to give calcium salt as a white solid (300 mg).

LCMS: purity: 95.41%; MS (m/e): 546.22 (MH+).

### VI-80: (4-(44(14(1,3-cis)-3-ethoxycyclobutyl)-3-(pyridin-2-yl)-1H-pyrazol-4-yl)carbamoyl)thiazol-2-yl)-1H-pyrazol-1-yl)methyl phosphate bis-ammonium salt.

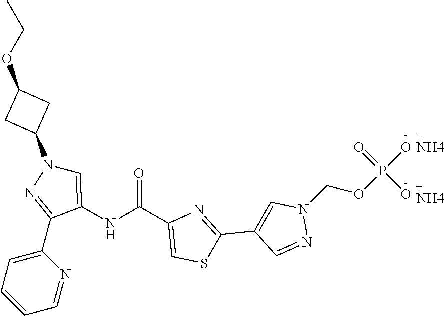

To a mixture of (4-(4-((1-((1,3-cis)-3-ethoxycyclobutyl)-3-(pyridin-2-yl)-1H-pyrazol-4-yl)carbamoyl)thiazol-2-yl)-1H-pyrazol-1-yl)methyl phosphate (200 mg) in acetonitrile (1 mL) and water (1 mL), was added 2.0 N ammonia in methanol solution (0.37 mL, 2 eq.). After sonicating for five minutes, the solution was lyophilized for 24 hours. The resulting powder was suspended in water (0.5 mL) and isopropanol (3 mL). The resulting precipitate was collected through filtration, washed with isopropanol (3×1 mL) and dried under high vacuum at room temperature for 24 hours to give ammonium salt (180 mg) as a white solid.

¹H NMR (300 MHz, Deuterium Oxide) 6 7.71 (s, 2H), 7.56 (s, 1H), 7.33 (m, 2H), 7.19 (s, 1H), 7.08 (d, J=8.1 Hz, 1H), 6.82 (t, J=5.7 Hz, 1H), 5.53 (d, J=7.8 Hz, 2H), 4.08 (p, J=7.8 Hz, 1H), 3.89 (m, 1H), 3.48 (q, J=7.2 Hz, 2H), 2.79 (m, 2H), 2.13 (m, 2H), 1.13 (t, J=7.2 Hz, 3H); LCMS: purity: 100%; MS (m/e): 546.15 (MH+).

### VI-81: (4-(4-((1-((1,3-cis)-3-ethoxycyclobutyl)-3-(pyridin-2-yl)-1H-pyrazol-4-yl)carbamoyl)thiazol-2-yl)-1H-pyrazol-1-yl)methyl phosphate bis-lysine salt.

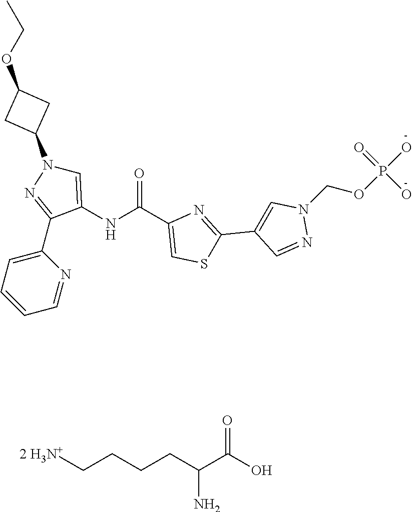

To a mixture of (4-(4-((1-((1,3-cis)-3-ethoxycyclobutyl)-3-(pyridin-2-yl)-1H-pyrazol-4-yl)carbamoyl)thiazol-2-yl)-1H-pyrazol-1-yl)methyl phosphate (200 mg) in acetonitrile (1 mL) and water (1 mL), was added L-lysine (107 mg, 2 eq.). After sonicating for five minutes, the solution was lyophilized for 24 hours. The resulting powder was suspended in water (0.5 mL) and isopropanol (3 mL). The resulting precipitate was collected through filtration, washed with isopropanol (3×1 mL) and dried under high vacuum at room temperature for 24 hours to give bis-lysine salt (200 mg) as a white solid.

¹H NMR (300 MHz, Deuterium Oxide) 6 7.82 (m, 1H), 7.79 (s, 1H), 7.63 (s, 1H), 7.41 (s, 1H), 7.39 (m, 1H), 7.28 (s, 1H), 7.16 (d, J=9.0 Hz, 1H), 6.88 (m, 1H), 5.56 (d, J=8.1 Hz, 2H), 4.12 (m, 1H), 3.90 (t, J=7.8 Hz, 1H), 3.61 (t, J=5.7 Hz, 2H), 3.48 (q, J=6.9 Hz, 2H), 2.88 (t, J=7.5 Hz, 4H), 2.82 (m, 2H), 2.16 (m, 2H), 1.80-1.72 (m, 4H), 1.63-1.53 (m, 4H), 1.42-1.29 (m, 4H), 1.13 (t, J=7.2 Hz, 3H); LCMS: purity: 100%; MS (m/e): 546.15 (MH+).

### VI-82: (4-(44(14(1,3-cis)-3-ethoxycyclobutyl)-3-(pyridin-2-yl)-1H-pyrazol-4-yl)carbamoyl)thiazol-2-yl)-1H-pyrazol-1-yl)methyl phosphate bis-arginine salt.

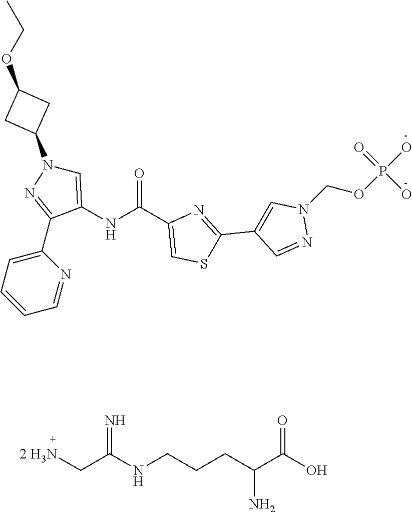

To a mixture of (4-(4-((1-((1,3-cis)-3-ethoxycyclobutyl)-3-(pyridin-2-yl)-1H-pyrazol-4-yl)carbamoyl)thiazol-2-yl)-1H-pyrazol-1-yl)methyl phosphate (200 mg) in acetonitrile (1 mL) and water (1 mL), was added L-arginine (128 mg, 2 eq.). After sonicating for five minutes, the solution was lyophilized for 24 hours. The resulting powder was suspended in water (0.5 mL) and isopropanol (3 mL). The resulting precipitate was collected through filtration, washed with isopropanol (3×1 mL) and dried under high vacuum at room temperature for 24 hours to give bis-arginine salt (200 mg) as a white solid. The salt was re-dissolved in water (0.5 mL) and acetone (8 mL). After heating at 50° C. for 10 minutes, the solution was cooled to room temperature. The resulting precipitate was collected through filtration, washed with acetone and dried under high vacuum at room temperature for 24 hours to give bis-arginine salt (120 mg) as a white solid.

¹H NMR (300 MHz, Deuterium Oxide) 6 7.88 (d, J=5.4 Hz, 1H), 7.84 (s, 1H), 7.68 (s, 1H), 7.46 (s, 1H), 7.41 (d, J=6.3 Hz, 1H), 7.33 (s, 1H), 7.20 (d, J=8.1 Hz, 1H), 6.92 (m, 1H), 5.57 (d, J=8.7 Hz, 2H), 4.15 (t, J=8.7 Hz, 1H), 3.91 (t, J=6.6 Hz, 1H), 3.62 (t, J=6.0 Hz, 2H), 3.49 (q, J=7.2 Hz, 2H), 3.08 (t, J=6.9 Hz, 4H), 2.82 (m, 2H), 2.11 (m, 2H), 1.80-1.72 (m, 4H), 1.63-1.44 (m, 4H), 1.14 (t, J=7.2 Hz, 3H); LCMS: purity: 100%; MS (m/e): 546.15 (MH+).

### VI-83: (4-(4-((1-((1,3-cis)-3-ethoxycyclobutyl)-3-(pyridin-2-yl)-1H-pyrazol-4-yl)carbamoyl)thiazol-2-yl)-1H-pyrazol-1-yl)methyl dihydrogen phosphate.

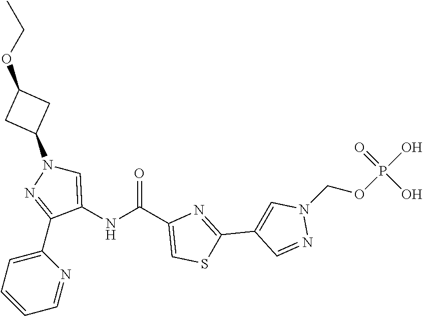 [SMILES: CCO[C@H]1C[C@@H](n2cc(NC(=O)c3csc(-c4cnn(COP(=O)(O)O)c4)n3)c(-c3ccccn3)n2)C1]

N-(1-((1,3-Cis)-3-ethoxycyclobutyl)-3-(pyridin-2-yl)-1H-pyrazol-4-yl)-2-(1H-pyrazol-4-yl)thiazole-4-carboxamide (59 g) and cesium carbonate (88 g, 2eq.) were suspended in dimethylformamide (500 mL), di-tert-butyl (chloromethyl) phosphate (53 g, 1.5 eq.) was added to the reaction and the mixture allowed to stir at room temperature for 16-20 hours. The reaction mixture was diluted with water (1 L) and extracted with ethyl acetate (2×800 mL). The combined organic layers were evaporated at room temperature and purified using the Torrent Combiflash®Rf column chromatography (ethyl acetate in hexanes, 20 to 100%) to give the prodrug ester as a colorless oil (85 g, 95% yield). LCMS: purity: 100%; MS (m/e): 658.38 (MH+).

Di-tert-butyl44-(4-41-((1,3-cis)-3-ethoxycyclobutyl)-3-(pyridin-2-yl)-1H-pyrazol-4-yl)carbamoyl)thiazol-2-yl)-1H-pyrazol-1-yl)methyl) phosphate (85 g) was dissolved in anhydrous dichloromethane (700 mL), the resulting solution was cooled to 0° C. and trifluoroacetic acid (150 mL) was added drop-wise. The reaction mixture was stirred at 0° C. for 6 hours, when LC-MS analysis showed full conversion to the acid, the solution was evaporated on a rotary evaporator at room temperature. The residue was dried further under high vacuum at room temperature for 24 hours to give a light yellow semi-solid as the acid and used subsequently to form salts.

(4-(4-((1-((1,3-cis)-3-ethoxycyclobutyl)-3-(pyridin-2-yl)-1H-pyrazol-4-yl)carbamoyl)thiazol-2-yl)-1H-pyrazol-1-yl)methyl dihydrogen phosphate (100 mg) was stirred overnight at 50° C. in acetone (10 mL) and water (0.5 mL). The cloudy solution was cooled to room temperature. The white precipitate was collected by filtration, washed with acetone and dried under high vacuum at room temperature for 24 hours (90 mg).

¹H NMR (300 MHz, DMSO-d6) 6 12.20 (s, 1H), 8.83 (d, J=4.8 Hz, 1H), 8.61 (s, 1H), 8.46 (s, 1H), 8.32 (s, 1H), 8.18 (s, 1H), 8.04 (d, J=8.1 Hz, 1H), 7.93 (t, J=6.9 Hz, 1H), 7.40 (t, J=6.0 Hz, 1H), 5.90 (d, J=11.1 Hz, 2H), 4.60 (t, J=8.4 Hz, 1H), 3.83 (t, J=6.6 Hz, 1H), 3.41 (q, J=6.9 Hz, 2H), 2.80 (m, 2H), 2.42 (m, 2H), 1.13 (t, J=6.9 Hz, 3H); LCMS: purity: 100%; MS (m/e): 546.15 (MH+).

### VI-84: (4-(44(14(1,3-cis)-3-ethoxycyclobutyl)-3-(pyridin-2-yl)-1H-pyrazol-4-yl)carbamoyl)thiazol-2-yl)-1H-pyrazol-1-yl)methyl phosphate Tris salt.

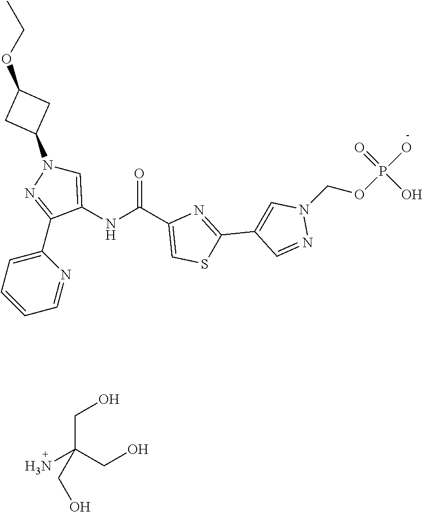 [SMILES: CCO[C@H]1C[C@@H](n2cc(NC(=O)c3csc(-c4cnn(COP(=O)(O)O)c4)n3)c(-c3ccccn3)n2)C1 ; NC(CO)(CO)CO]

To a mixture of (4-(4-((1-((1,3-cis)-3-ethoxycyclobutyl)-3-(pyridin-2-yl)-1H-pyrazol-4-yl)carbamoyl)thiazol-2-yl)-1H-pyrazol-1-yl)methyl phosphate (118 mg) in acetonitrile (1 mL) and water (1 mL), was added Tris(hydroxymethyl)aminomethane (52 mg, 2 eq.). After sonicating for five minutes, the solution was lyophilized for 24 hours. The resulting powder was suspended in water (0.5 mL) and acetone (5 mL). The solution was stirred at 50° C. for 30 minutes and cooled to room temperature. After one week at room temperature, the resulting precipitate was collected through filtration, washed with acetone (3×1 mL) and dried under high vacuum at room temperature for 24 hours to give mono-Tris salt (120 mg) as a white solid.

¹H NMR (300 MHz, Deuterium Oxide) 6 7.83 (m, 2H), 7.65 (s, 1H), 7.43 (s, 1H), 7.40 (d, J=7.5 Hz, 1H), 7.30 (s, 1H), 7.17 (d, J=8.1 Hz, 1H), 6.90 (t, J=6.0 Hz, 1H), 5.57 (d, J=8.1 Hz, 2H), 4.13 (t, J=7.5 Hz, 1H), 3.91 (t, J=6.9 Hz, 1H), 3.60 (s, 6H), 3.49 (q, J=6.9 Hz, 2H), 2.82 (m, 2H), 2.18 (m, 2H), 1.14 (t, J=6.9 Hz, 3H); LCMS: purity: 100%; MS (m/e): 546.16 (MH+).

Compounds V-1 to V-156 and VI-1 to VI-180 were made by methods similar to those described herein and/or known to persons of ordinary skill in the art. Additional information concerning these compounds can be found in U.S. Pat. No. 9,982,000 which is incorporated herein by reference in its entirety.

### Example 3

### Synthesis of Pyrazole Compounds According to Formula VII

### Formation of N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide Benzenesulfonic Acid Salt

### (VII-65)

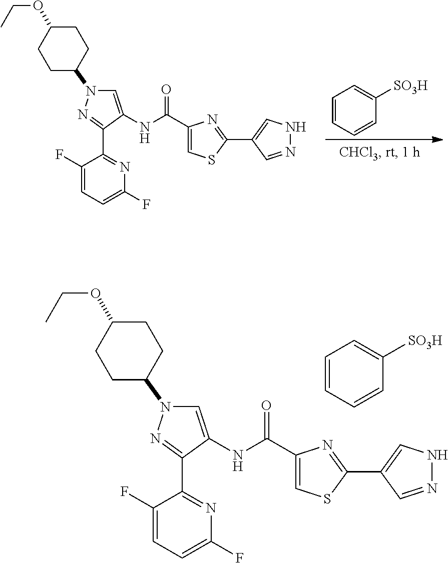

N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide (0.050 g, 0.100 mmol, 1.0 eq) was dissolved in chloroform (1.0 eq) to obtain a clear colorless solution. Benzenesulfonic acid (0.019 g, 0.120 mmol, 1.2 eq) was added and a precipitate formed over the next 15 minutes. The reaction was stirred at room temperature for 1 hour and the precipitate was isolated by filtration to obtain the title compound (0.038 g) as a white solid; ¹H nmr (400 MHz, D₆-DMSO) δ 8.53 (1H, s, thiazoleH-5 or pyrazoleH-5), 8.30 (1H, s, 1H of thiazoleH-5 or pyrazoleH-5, pyrazoleH-3, H-5), 8.29 (1H, s, 1H of thiazoleH-5 or pyrazoleH-5, pyrazoleH-3, H-5), 8.28 (1H, s, 1H of thiazoleH-5 or pyrazoleH-5, pyrazoleH-3, H-5), 8.08 (1H, dt, J 9.0, 6.5 Hz, pyridineH-4 or H-5), 7.59-7.56 (2H, m, 2H of C₆H₅SO₃H), 7.32-7.27 (4H, m, pyridineH-4 or H-5, 3H of C₆H₅SO₃H), 4.33 (1H, tt, J 11.5, 3.5 Hz, cyclohexaneH-1 or H-4), 3.47 (2H, q, J 7.0 Hz, OCH₂CH₃), 3.34 (1H, tt, J 10.5, 3.5 Hz, cyclohexaneH-1 or H-4), 2.08 (4H, m, 4H of cyclohexaneH-2, H-3, H-5, H-6), 1.85 (2H, m, cyclohexaneH-2, H-3, H-5, H-6), 1.35 (2H, m, 2H of cyclohexaneH-2, H-3, H-5, H-6), 1.10 (3H, t, J 7.0 Hz, OCH₂CH₃); ¹⁹F nmr (380 MHz, D6-DMSO) δ −73.0 (dd, 24.5, 2.5 Hz), −124.2 (ddd, J 26.0, 9.5, 1.5 Hz); m/z: 500 [M+H]⁺.

### Formation of N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide Sodium Salt (VII-67)

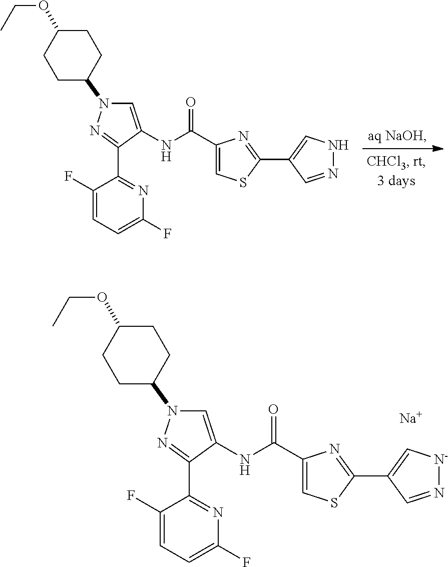

N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide (0.062 g, 0.124 mmol, 1.0 eq) was dissolved in chloroform (2.0 mL) to obtain a clear solution. Sodium hydroxide (0.05 mL of a 3M aqueous solution, 0.149 mmol, 1.2 eq) was added and the reaction was stirred at room temperature for 3 days. No precipitate was formed. The reaction was concentrated and further concentrated from acetonitrile (5 mL) to obtain the title compound as a white solid;

¹H nmr (400 MHz, D6-DMSO) δ 8.53 (1H, s, thiazoleH-5 or pyrazoleH-5), 8.13 (3H, br s, thiazoleH-5 or pyrazoleH-5, pyrazoleH-3, H-5), 8.08 (1H, dt, J 9.5, 6.5 Hz, pyridineH-4 or H-5), 7.28 (1H, ddd, J 9.0, 3.0, 2.5 Hz, pyridineH-4 or H-5), 4.33 (1H, tt, J 11.5, 3.0 Hz, cyclohexaneH-1 or H-4), 3.47 (2H, q, J 7.0 Hz, OCH₂CH₃), 3.35 (1H, tt, J 11.0, 3.5 Hz, cyclohexaneH-1 or H-4), 2.08 (4H, m, 4H of cyclohexaneH-2, H-3, H-5, H-6), 1.85 (2H, m, cyclohexaneH-2, H-3, H-5, H-6), 1.35 (2H, m, 2H of cyclohexaneH-2, H-3, H-5, H-6), 1.10 (3H, t, J 7.0 Hz, OCH₂CH₃); m/z: 500 [M+H]⁺.

### Formation of N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide tartaric acid cocrystal (VII-66)

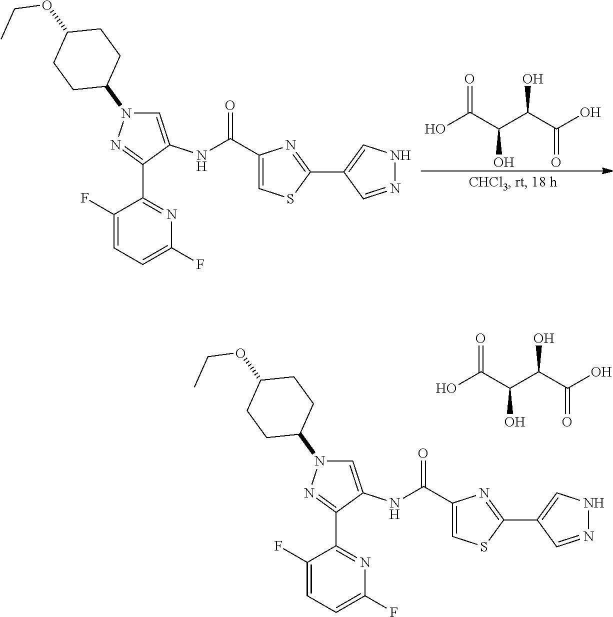

L-Tartaric acid (0.017 g, 0.110 mmol, 1.1 eq) was added to a solution of N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide (0.050 g 0.100 mmol, 1.0 eq) in chloroform (1.0 eq). A white solid slowly precipitated. The reaction was stirred at room temperature for 18 hours and the precipitate isolated by filtration to obtain the title compound (0.055 g, 85%) as a white solid; ¹H nmr (400 MHz, D6-DMSO) δ 8.53 (1H, s, thiazoleH-5 or pyrazoleH-5), 8.29 (3H, br s, thiazoleH-5 or pyrazoleH-5, pyrazoleH-3, H-5), 8.08 (1H, dt, J 9.5, 6.5 Hz, pyridineH-4 or H-5), 7.28 (1H, dt, J 9.0, 3.0 Hz, pyridineH-4 or H-5), 5.05 (2H, br s, 2× OH), 4.33 (1H, tt, J 11.5, 3.5 Hz, cyclohexaneH-1 or H-4), 4.29 (2H, s, COCH(OH)CH(OH)CO), 3.47 (2H, q, J 7.0 Hz, OCH₂CH₃), 3.34 (1H, tt, J 10.5, 3.5 Hz, cyclohexaneH-1 or H-4), 2.08 (4H, m, 4H of cyclohexaneH-2, H-3, H-5, H-6), 1.85 (2H, m, cyclohexaneH-2, H-3, H-5, H-6), 1.35 (2H, m, 2H of cyclohexaneH-2, H-3, H-5, H-6), 1.09 (3H, t, J 7.0 Hz, OCH₂CH₃); ¹³C nmr (100 MHz, D6-DMSO) δ 173.5, 161.7, 157.7, 157.6 (d, J 236.0 Hz), 153.5 (dd, J 259.0, 4.0 Hz), 149.2, 138.2 (t, J 15.0 Hz), 132.6 (d, J 9.0 Hz), 131.9 (dd, J 22.5, 9.0 Hz), 123.5, 121.5, 120.2, 116.2, 109.2 (dd, J 43.0, 8.5 Hz), 76.0, 72.6, 63.0, 60.8, 30.9, 30.9, 16.1; ¹⁹F nmr (380 MHz, D6-DMSO) δ −73.0, −124.2; m/z: 500 [M+H]⁺.

### Formation of N-(3-(3,6-difluoropyridin-2-yl)-1-(trans-4-ethoxycyclohexyl)-1H-pyrazol-4-yl)-2-(1H-pyrazol-4-yl)thiazole-4-carboxamide hemi((2R,3R)-2,3-dihydroxysuccinate) (VII-11)

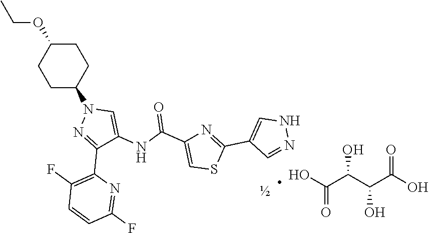

A MeOH (1.3 mL) solution of (L)-Tartaric Acid (750.5 mg, 5 mmol) was added dropwise to a CH₂CL₂-MeOH (60 mL-5 mL) solution of N-(3-(3,6-difluoropyridin-2-yl)-1-(trans-4-ethoxycyclohexyl)-1H-pyrazol-4-yl)-2-(1H-pyrazol-4-yl)thiazole-4-carboxamide (5.0 g, 10 mmol) at 35° C., additional MeOH (5 mL) and CH₂Cl₂ (100 mL) were added after 15 minutes. The mixture was stirred at 35° C. for another 20 hours, and then cooled to room temperature. Solid was collected by filtration, washed with CH₂Cl₂, and was further dried in vacuo. The title compound was obtained as a white solid: 3.48 g (60.7% yield); ¹H NMR (400 MHz, DMSO-d₆) δ 13.32 (br s, 1H), 12.74 (br s, 1H), 11.45 (s, 1H), 8.51 (s, 1H), 8.27 (s, 1H), 8.43-8.14 (m, 2H), 8.07 (ddd, J=9.8, 8.8, 6.3 Hz, 1H), 7.27 (ddd, J=8.8, 2.9, 2.9 Hz, 1H), 5.07 (br s, 1H), 4.31 (tt, partially overlapped, J=11.7, 3.2 Hz, 1H), 4.27 (s, 1H), 3.45 (q, J=7.0 Hz, 2H), 3.33 (tt, partially overlapped with H₂O, J=10.7, 3.6 Hz, 1H), 2.08-2.03 (m, 4H), 1.88-1.78 (m, 2H), 1.38-1.28 (m, 2H), 1.08 (t, J=7.0 Hz, 3H); ¹⁹F NMR (376 MHz, DMSO-d₆) δ −72.97 (ddd, J=28.1, 6.8, 3.8 Hz), −124.18 (ddd, J=28.1, 10.3, 3.2 Hz); LRMS (M+H) m/z 500.2.

A second crop (1.58 g, combined yield: 88%) of the same compound was obtained from the filtrate, after removal of the solvent in vacuo, and resuspended the solid in CH₂Cl₂-MeOH (25 mL-2 mL) at 35° C. overnight.

### Preparation of N-(3-(3,6-difluoropyridin-2-yl)-1-(trans-4-ethoxycyclohexyl)-1H-pyrazol-4-yl)-2-(1H-pyrazol-4-yl)thiazole-4-carboxamide (VII-1)- Method 1

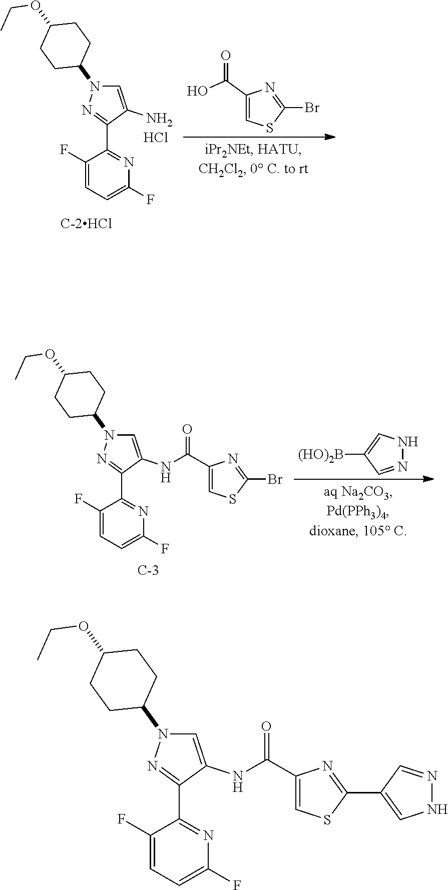

### I. Preparation of 2-bromo—N-(3-(3,6-difluoropyridin-2-yl)-1-(trans-4-ethoxycyclohexyl)-1H-pyrazol-4-yl)thiazole-4-carboxamide C-3 from C_{2.}HCl

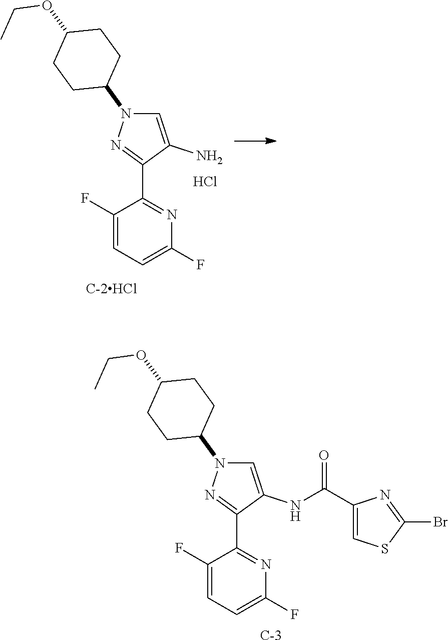

Diisopropylethylamine (8.5 mL, 48.95 mmol, 3.5 eq) was added to a mixture of the aminopyrazole C-2.HCl (5.00 g, 13.99 mmol, 1.0 eq) and bromothiazolecarboxylic acid (3.20 g, 15.38 mmol, 1.1 eq) in dichloromethane (50 mL) at 0° C. HATU (5.85 g, 15.38 mmol, 1.1 eq) added. The reaction was stirred at 0° C. for 10 minutes and then at room temperature for 4 hours. The reaction was diluted with CH₂Cl₂ (100 mL). The organics were washed with NaHCO₃ (150 mL), NH₄Cl (150 mL) and brine (100 mL), dried (Na₂SO₄) and concentrated under reduced pressure. The residue was suspended in EtOAc-hexane (1:1, 50 mL) and the resulting solid was isolated by filtration. The solid was suspended in NaHCO₃ (50 mL) for 1 hour to remove residual coupling agent before isolating by filtration and drying under vacuum to obtain C-3 (5.3 g, 74%) as an off-white solid; IR vₘₐₓ (film) 3290, 3121, 2942, 2865, 1671, 1615, 1552, 1485, 1431, 1377, 1237, 1154, 1104, 1056, 1011, 819, 787, 731 cm^{-1; 1}H nmr (400 MHz, CDCl₃) δ 8.42 (1H, d, J 0.5 Hz, thiazoleH-5 or pyrazoleH-5), 8.09 (1H, s, thiazoleH-5 or pyrazoleH-5), 7.63 (1H, td, J 9.0, 6.0 Hz, pyridineH-4 or H-5), 6.85 (1H, ddd, J 9.0, 3.5, 2.5 Hz, pyridineH-4 or H-5), 4.26 (1H, tt, J 11.5, 4.0 Hz, cyclohexaneH-1 or H-4), 3.55 (2H, q, J 7.0 Hz, OCH₂CH₃), 3.36 (1H, tt, J 10.5, 4.0 Hz, cyclohexaneH-1 or H-4), 2.28 (2H, br d, J 13.0 Hz, 2H of cyclohexaneH-2, H-3, H-5, H-6), 2.21 (2H, m, 2H of cyclohexaneH-2, H-3, H-5, H-6), 1.91, 1.84 (2H, 2dd AB system, J 13.0, 3.5 Hz, 2H of cyclohexaneH-2, H-3, H-5, H-6), 1.46 (2H, m, 2H of cyclohexaneH-2, H-3, H-5, H-6), 1.22 (3H, t, J 7.0 Hz, OCH₂CH₃); ¹³C nmr (100 MHz, CDCl₃) δ 157.6 (d, J 238.0 Hz), 156.9, 153.3 (dd, J 260.0, 8.5 Hz), 150.0, 138.6 (t, J 14.0 Hz), 136.1, 133.1 (d, J 8.5 Hz), 129.8 (dd, J 23.0, 8.5 Hz), 126.7, 121.7, 119.2, 107.8 (dd, J 39.5, 5.5 Hz), 76.4, 63.6, 61.5, 31.1, 30.9, 15.7;¹⁹F nmr (380 MHz, CDCl₃) δ −72.3, −124.9; m/z: 536, 534 [M+Na]⁺, 514, 512 [M+H]⁺. The filtrate from the initial trituration was purified by column chromatography (20→80% EtOAc-hexane) to obtain further C-3 (0.8 g, 9%) as a pink foam.

### II. Preparation of N-(3-(3,6-difluoropyridin-2-yl)-1-(trans-4-ethoxycyclohexyl)-1H-pyrazol-4-yl)-2-(1H-pyrazol-4-yl)thiazole-4-carboxamide

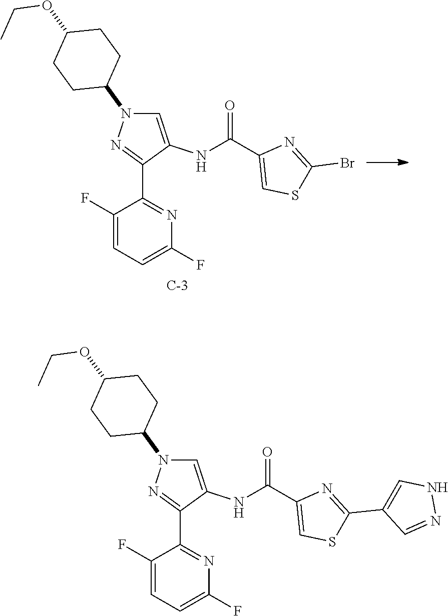

Dioxane (400 mL) was added to a mixture of the bromothiazole C-3 (25.0 g, 48.8 mmol, 1.0 eq) and pyrazole-4-boronic acid (8.2 g, 73.2 mmol, 1.5 eq) followed by aqueous solution of sodium carbonate (73.3 mL of a 2M solution, 146.5 mmol, 3.0 eq). The reaction mixture was degassed by bubbling argon through for five minutes. Tetrakis(triphenylphosphine)palladium (1.4 g, 1.2 mmol, 0.025 eq) was added and the reaction further degassed before heating to 105° C. for 6 hours. The reaction was filtered through celite® while hot, eluting with EtOAc (200 mL). The filtrate was concentrated to approximately 150 mL, upon which a precipitate formed. The precipitate was isolated by filtration. The filtrate was concentrated to remove the remaining organics, filtered to remove more precipitate, diluted with water-brine (1:2,300 mL) and extracted with EtOAc (3×200 mL). The combined organics were combined, dried (Na₂SO₄) and concentrated under reduced pressure. The combined precipitates and extracts were loaded onto silica. Column chromatography (silica, 0→10% MeOH—CH₂Cl₂) yielded the title compound (16.5 g, 68%) as a white solid; IR vₘₐₓ (film) 3229, 2938, 2861, 1663, 1615, 1589, 1549, 1482, 1425, 1377, 1237, 1104, 1055, 972, 930, 903, 875, 820, 786, 715, 664 cm1; ^{1H} nmr (400 MHz, CDCl₃) δ 8.52 (1H, s, thiazoleH-5 or pyrazoleH-5), 8.24 (2H, s, NHpyrazoleH-3, H-5), 8.07 (1H, s, thiazoleH-5 or pyrazoleH-5), 7.41 (1H, td, J 9.0, 6.0 Hz, pyridineH-4 or H-5), 6.86 (1H, ddd, J 9.0, 3.5, 2.5 Hz, pyridineH-4 or H-5), 4.28 (1H, tt, J 11.5, 4.0 Hz, cyclohexaneH-1 or H-4), 3.57 (2H, q, J 7.0 Hz, OCH₂CH₃), 3.37 (1H, tt, J 11.0, 4.0 Hz, cyclohexaneH-1 or H-4), 2.26 (4H, m, 4H of cyclohexaneH-2, H-3, H-5, H-6), 1.92, 1.86 (2H, 2dd AB system, J 13.0, 3.5 Hz, 2H of cyclohexaneH-2, H-3, H-5, H-6), 1.50, 1.44 (2H, 2dd AB system, J 13.0, 3.5 Hz, 2H of cyclohexaneH-2, H-3, H-5, H-6), 1.23 (3H, t, J 7.0 Hz, OCH₂CH₃); ¹³C nmr (100 MHz, CDCl₃) δ 160.6, 158.6, 158.3, 156.3, 154.8, 152.2, 150.2, 138.9, 133.0 (d, J 9.0 Hz), 129.9 (dd, J 23.5, 9.0 Hz), 122.0, 121.6, 119.4, 117.2, 107.5 (dd, J 40.5, 5.0 Hz), 76.4, 63.7, 61.5, 31.1, 30.9, 15.7; ¹⁹F nmr (380 MHz, CDCl₃) 6-72.7 (dddd, J 27.0, 9.5, 5.5, 4.0 Hz), −124.3 (ddd, J 27.5, 9.5, 3.0 Hz); m/z: 500 [M+H]⁺ (found [M+H]⁺, 500.1687, C₂₃H₂₃F₂N₇O₂S requires [M+H]⁺ 500.1675).

### Preparation of N-(3-(3,6-difluoropyridin-2-yl)-1-(trans-4-ethoxycyclohexyl)-1H-pyrazol-4-yl)-2-(1H-pyrazol-4-yl)thiazole-4-carboxamide (VII-1)- Method 2

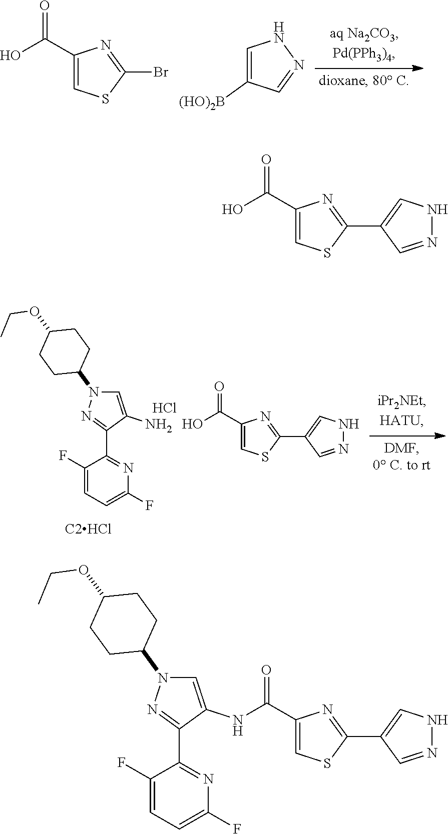

### Formation of 2-(1H-pyrazol-4-yl)thiazole-4-carboxylic acid

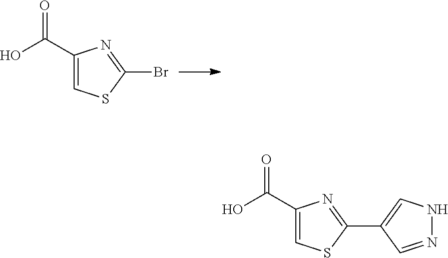 [SMILES: O=C(O)c1csc(Br)n1 ; O=C(O)c1csc(-c2cn[nH]c2)n1]

A 1,4-Dioxane-H₂O (32 mL-8 mL) solution of 2-bromothiazole-4-carboxylic acid (2.08 g, 10 mmol, 1.0 eq), (1H-pyrazol-4-yl)boronic acid (3.36 g, 30 mmol, 3.0 eq), tetrakis(triphenylphosphine)palladium (0.23 g, 0.2 mmol, 0.02 eq) and sodium carbonate (3.18 g, 30 mmol, 3.0 eq) was degassed, backed-filled with nitrogen gas, three times. The cloudy solution was stirred at 60° C. for 2 hours (by LC-MS, starting material : product≈1:1), then at 100° C. for a further 3 hours, until the reaction went to completion as monitored by LC-MS. After removal of organic solvent under reduced pressure, the crude mixture was diluted with water (100 mL) and mixed well. The aqueous solution was passed through a celite® pad, and washed with water. While stirring, the filtrate with acidified with 6M HCl aq. solution (about 11 mL) until pH=1-2. The precipitate was collected by filtration, washed with water and further dried in vacuo to obtain the title compound (1.79 g 92% yield) as a light tan color solid; ¹H nmr (400 MHz, D6-DMSO) δ 13.11 (2H, br s, NH, OH), 8.28 (1H, s, thiazoleH-4), 8.17 (2H, br s, pyrazoleH-3, H-5); m/z: 196 [M+H]⁺.

### II. Preparation of N-(3-(3,6-difluoropyridin-2-yl)-1-(trans-4-ethoxycyclohexyl)-1H-pyrazol-4-yl)-2-(1H-pyrazol-4-yl)thiazole-4-carboxamide (VII-1)

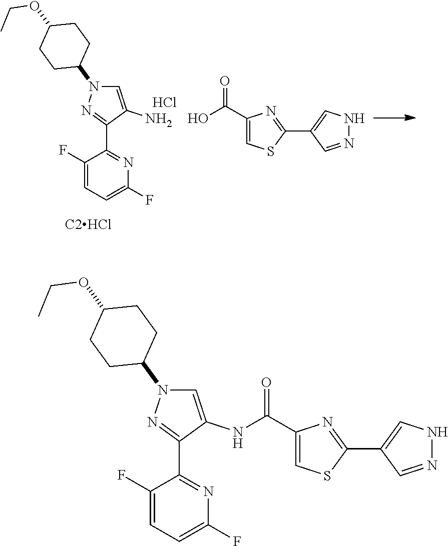

A mixture of the C_{2.}HCl aminopyrazole hydrochloride (1.00 g, 2.80 mmol, 1.0 eq) and 2-(1H-pyrazol-4-yl)thiazole-4-carboxylic acid (0.65 g, 3.36 mmol, 1.2 eq) in dimethylformamide (14 mL) was cooled to 0° C. and diisopropylethylamine (1.22 mL, 6.99 mmol, 2.5 eq) added. A solution resulted to which was added HATU (1.17 g, 3.08 mmol, 1.1 eq). The solution was stirred at 0° C. for 15 minutes and room temperature for 1 hour, before adding the reaction to water (75 mL). A solid formed that collapsed to a gum. The liquid was decanted isolating any solid by filtration. The gum and solid were dissolved in Et0Ac-MeOH (4:1, 100 mL), combined and concentrated under reduced pressure. The resulting solid was triturated from 10% EtOH-EtOAc (4 mL) to obtain the title compound VII-1 as an off-white solid (0.76 g, 55%). The filtrate was concentrated and loaded onto silica. Column chromatography (0→10% MeOH-CH₂Cl₂) yielded a pale yellow solid, which was stirred with NaHCO₃ (15 mL). The liquid was decanted and the residue triturated with 10% EtOH-EtOAc (4 mL) to obtain further product as an off-white solid (0.226 g, 16%). Total yield 0.99 g, 71%; data agreed with that stated above.

### Exemplary Synthesis of Alkyl Phosphate Compounds

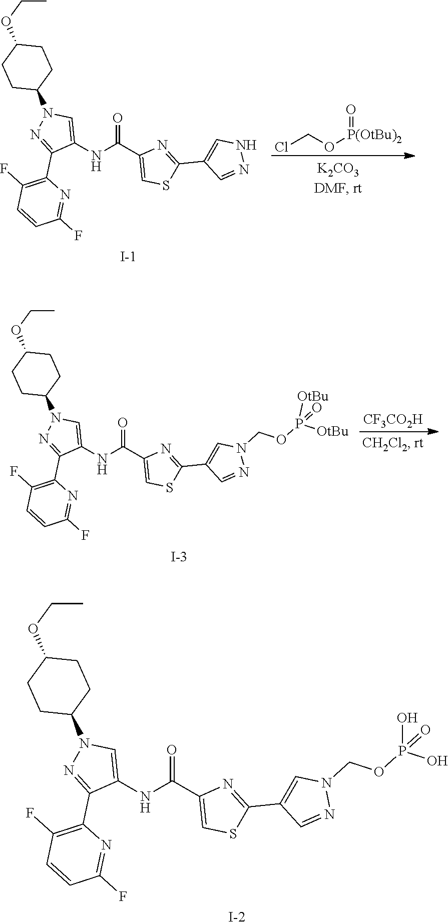

### I. Preparation of di-tert-butyl ((4-(4-((3-(3,6-difluoropyridin-2-yl)-1-(trans-4-ethoxycyclohexyl)-1H-pyrazol-4-yl)carbamoyl)thiazol-2-yl)-1H-pyrazol-1-yl)methyl) phosphate (VII-3)

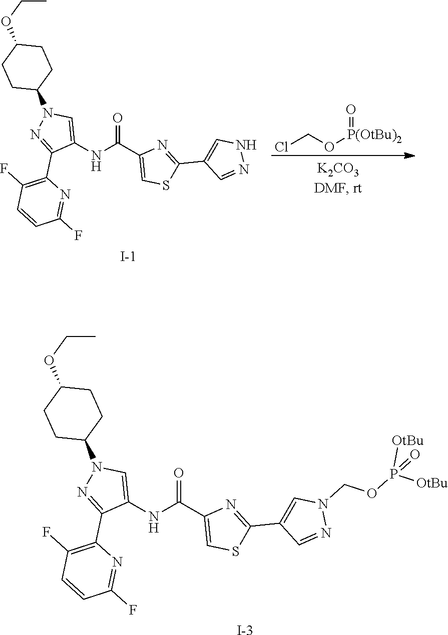

Potassium carbonate (0.41 g, 3.01 mmol, 1.5 eq) was added to a suspension of VII-1 (1.00 g, 2.00 mmol, 1.0 eq) in dimethylformamide (14 mL). The reaction was stirred at room temperature for 30 minutes before adding a solution of chloromethyl di-tert-butyl phosphate (1.04 g, 4.01 mmol, 2.0 eq) in dimethylformamide (2 mL). The reaction was stirred at room temperature for 14 hours. Further chloromethyl di-tert-butyl phosphate (0.52 g, 2.00 mmol, 1.0 eq) and potassium carbonate (0.21 g, 1.50 mmol, 0.75 eq) was added and the reaction stirred for a further 24 hours. The reaction was cooled to 0° C. and water (25 mL) added dropwise over 45 minutes. A sticky solid resulted which was isolated by decanting the liquid. The liquid was added to water (40 mL) and stirred to obtain more solid, which was isolated by filtration. The solid was dried under vacuum and used without further purification (1.76 g, quantitative—theoretical yield 1.44 g); IR vₘₐₓ (film) 3308, 2979, 2978, 2864, 1668, 1615, 1592, 1549, 1482, 1374, 1266, 1234, 1104, 998, 965, 822, 787, 714, 666 cm⁻¹; ¹H nmr (400 MHz, CDCl₃) δ 8.50 (1H, s, pyrazoleH-5, thiazoleH-5), 8.34 (1H, s, 1H of pyrazoleH-3, H-5), 8.21 (1H, s, 1H of pyrazoleH-3, H-5), 8.06 (1H, s 1H of pyrazoleH-5, thiazoleH-5), 7.65 (1H, td, J 9.0, 6.0 Hz, pyridineH-4 or H-5), 6.88 (1H, ddd, J 9.0, 3.0, 2.5 Hz, pyridineH-4 or H-5), 5.93 (2H, d, J 12.5 Hz, NCH₂OP), 4.27 (1H, tt, J 12.0, 4.0 Hz, cyclohexaneH-1 or H-4), 3.56 (2H, q, J 7.0 Hz, OCH₂CH₃), 3.37 (1H, tt, J 10.5, 4.0 Hz, cyclohexaneH-1 or H-4), 2.29 (2H, br d, J 12.5 Hz, 2H of cyclohexaneH-2, H-3, H-5, H-6), 2.22 (2H, br d, J 11.0 Hz, 2H of cyclohexaneH-2, H-3, H-5, H-6), 1.89 (2H, m, 2H of cyclohexaneH-2, H-3, H-5, H-6), 1.50 (2H, m, 2H of cyclohexaneH-2, H-3, H-5, H-6), 1.45 (18H, s, 2× OC(CH₃)3), 1.22 (3H, t, J 7.0 Hz, OCH₂CH₃); ¹³C nmr (100 MHz, CDCl₃) δ 160.0, 158.2, 157.5 (d, J 236.5 Hz), 153.5 (dd, J 260.0, 5.0 Hz), 150.2, 139.5 (d, J 6.0 Hz), 138.9 (t, J 15.0 Hz), 133.0 (d, J 9.0 Hz), 130.0 (d, J 4.5 Hz), 129.8 (d, J 9.0 Hz), 122.0, 121.8, 119.4,5 118.6, 107.6 (dd, J 40.5, 5.0 Hz), 83.9, 83.8, 77.2, 76.4, 63.6, 61.5, 31.1, 30.9, 29.8, 29.7, 15.7; ³¹P nmr (162 MHz, CDCl₃) δ −11.1; ¹⁹F nmr (380 MHz, CDCl₃) δ −72.4 (dt, J 27.0, 5.5 Hz), −124.5 (dd, J 27.5, 9.5 Hz); m/z: 744 [M+Na]⁺.

### II. Preparation of (4-(4-((3-(3,6-difluoropyridin-2-yl)-1-(trans-4-ethoxycyclohexyl)-1H-pyrazol-4-yl)carbamoyl)thiazol-2-yl)-1H-pyrazol-1-yl)methyl dihydrogen phosphate (VII-2)

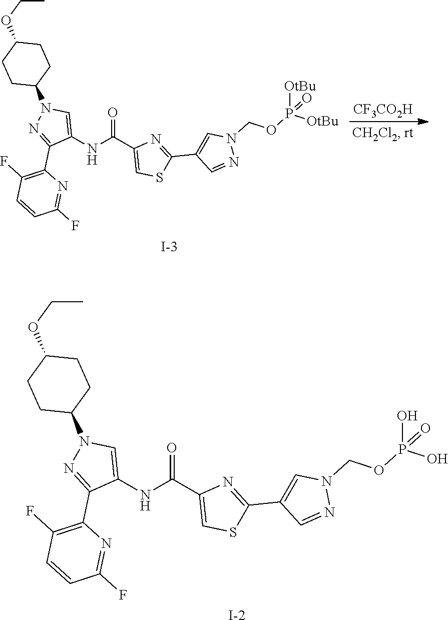

To a solution of VII-3 (1.58 g crude mass, 1.80 mmol, 1.0 eq) in dichloromethane (8.0 mL) was added trifluoroacetic acid (0.99 mL, 12.80 mmol, 7.1 eq). The reaction was stirred at room temperature for 20 hours, during which time a precipitate formed. After 20 hours the precipitate was isolated by filtration. The solid was washed with CH₂Cl₂ (2×8 mL) to obtain a white solid. The solid was stirred with dioxane-water (10:1, 11 mL) for 5 hours and filtered, washing with dioxane-water (10:1, 11 mL) to obtain VII-2 (0.60 g, 55% over two steps) as a white solid. The filtrate was concentrated and stirred in dioxane-water (10:1, 11 mL) for 18 hours before isolating by filtration. The solid was washed with dioxane-water (10:1, 2×5.5 mL) to obtain further product (0.12 g, total 0.72 g, 66%) as a white solid; ¹H nmr (400 MHz, D6-DMSO) δ 8.59 (1H, s, 1H of pyrazoleH-3, H-5), 8.52 (1H, s, 1H of pyrazoleH-3, H-5), 8.34 (1H, s, 1H of pyrazoleH-5, thiazoleH-5), 8.19 (1H, s, 1H of pyrazoleH-5, thiazoleH-5), 8.08 (1H, td, J 9.5, 6.5 Hz, pyridineH-4 or H-5), 6.88 (1H, ddd, J 9.0, 3.0, 2.5 Hz, pyridineH-4 or H-5), 5.83 (2H, d, J 12.5 Hz, NCH₂OP), 4.33 (1H, tt, J 12.0, 3.0 Hz, cyclohexaneH-1 or H-4), 3.47 (2H, q, J 7.0 Hz, OCH₂CH₃), 3.35 (1H, tt, J 10.5, 3.5 Hz, cyclohexaneH-1 or H-4), 2.29 (4H, br d, J 11.0 Hz, 4H of cyclohexaneH-2, H-3, H-5, H-6), 1.85 (2H, m, 2H of cyclohexaneH-2, H-3, H-5, H-6), 1.35 (2H, m, 2H of cyclohexaneH-2, H-3, H-5, H-6), 1.10 (3H, t, J 7.0 Hz, OCH₂CH₃); ¹³C nmr (100 MHz, CDCl₃) δ 160.6, 157.6, 157.6 (d, J 234.5 Hz), 154.3 (dd, J 259.5, 4.0 Hz), 149.4, 137.7 (d, J 7.0 Hz), 138.2, 132.6 (d, J 9.0 Hz), 131.9 (dd, J 22.0, 9.0 Hz), 131.4, 124.1, 121.4, 120.2, 117.7, 109.2 (d, 38.0 Hz), 76.0, 75.2, 63.0, 60.8, 30.9 (2C), 16.1; ³¹P nmr (162 MHz, D6-DMSO) δ −2.7; ¹⁹F nmr (380 MHz, D6-DMSO) δ −72.8, −124.2 (ddd, J 27.0, 9.5, 3.0 Hz); m/z: 610 [M+H]⁺ (found [M+H]⁺, 610.1451, C₂₄H₂₆F2N706PS requires [M+H]⁺ 610.1444).

Other phosphate compounds were made by similar methods

### Exemplary Synthesis of Carbamates and Ureas as Potential IRAK ProDrugs

### I. Formation of 2-morpholinoethyl (4-nitrophenyl) carbonate

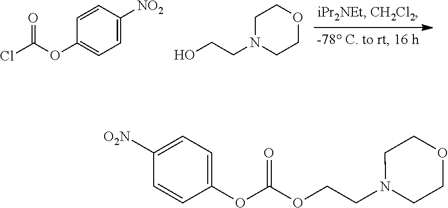 [SMILES: O=C(Cl)Oc1ccc([N+](=O)[O-])cc1 ; OCCN1CCOCC1 ; O=C(OCCN1CCOCC1)Oc1ccc([N+](=O)[O-])cc1]

A solution of 4-nitrophenol chloroformate (0.500 g, 2.48 mmol, 1.0 eq) in dichloromethane (20 mL) was cooled to −78° C. Diisopropylethylamine (0.65 mL, 3.72 mmol, 1.5 eq) was added followed by 4-(2-hydroxyethyl)morpholine (0.30 mL, 2.48 mmol, 1.0 eq) and the reaction was stirred between −78° C. and room temperature over 16 hours. The reaction was diluted with dichloromethane (40 mL) and washed with NaHCO₃ (60 mL) and brine (60 mL), dried (Na₂SO₄) and concentrated under reduced pressure to obtain the title compound as an orange oil; ¹H nmr (400 MHz, CDCl₃) δ 8.27 (2H, d, J 9.5 Hz, 2H of C₆H₄NO₂), 7.37 (2H, d, J 9.0 Hz, 2H of C₆H₄NO₂), 4.39 (2H, t, J 5.5 Hz, 2H of COOCH₂CH₂N), 3.72,3.71 (4H, 2d AB system, J 4.5 Hz, 4H of morpholine), 2.72 (2H, t, J 5.5 Hz, 2H of COCH₂CH₂N), 2.54, 2.53 (4H, 2d AB system , J 4.5 Hz, 4H of morpholine).

### II. Formation of 3-morpholinopropyl (4-nitrophenyl) carbonate

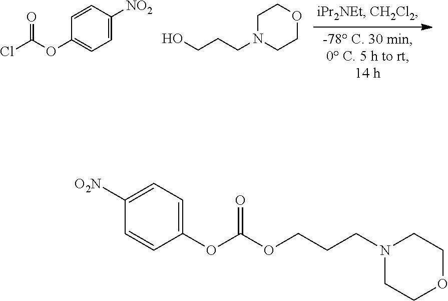 [SMILES: O=C(Cl)Oc1ccc([N+](=O)[O-])cc1 ; OCCCN1CCOCC1 ; O=C(OCCCN1CCOCC1)Oc1ccc([N+](=O)[O-])cc1]

Diisopropylethylamine (0.65 mL, 3.72 mmol, 1.5 eq) was added to a solution of 4-nitrophenyl chloroformate (0.500 g, 2.48 mmol, 1.0 eq) in dichloromethane (20 mL) at -78° C. 3-(Hydroxypropyl)morpholine (0.34 mL, 2.48 mmol, 1.0 eq) was added dropwise and the reaction stirred at −78° C. for 30 minutes. The reaction froze and was warmed to 0° C. After stirred at 0° C. for 5 hours the reaction was allowed to warm to room temperature over 16 hours. The reaction was diluted with dichloromethane (20 mL) and washed with NaHCO₃ (3×40 mL). The organics were dried (Na₂SO₄) and concentrated under reduced pressure to obtain the title compound as a pale yellow oil; ¹H nmr (400 MHz, CDCl₃) δ 8.26 (2H, d, J 9.5 Hz, 2H of C₆H₄NO₂), 7.36 (2H, d, J 9.0 Hz, 2H of C₆H₄NO₂), 4.36 (2H, t, J 6.5 Hz, OCH₂CH₂CH₂N), 3.70 3.69 (4H, 2d AB system, J 4.5 Hz, 4H of morpholine), 2.49-2.43 (6H, m, 4H of morpholine, OCH₂CH₂CH₂N), 1.93 (pentet, J 6.5 Hz, OCH₂CH₂CH₂N).

### III. Formation of 2-morpholinoethyl 4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazole-1-carboxylate (VII-10)

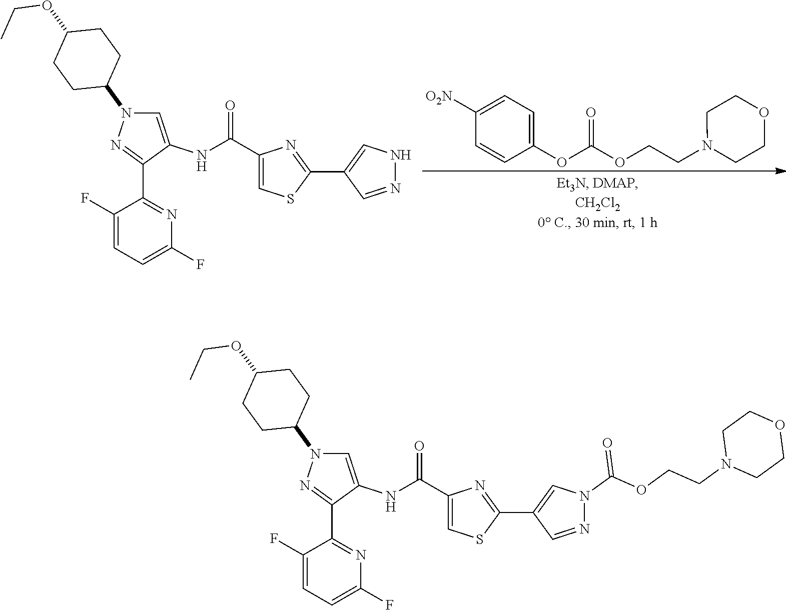

To the nitrophenyl carbonate (0.050 g, 0.169 mmol, 1.5 eq) in dichloromethane (1.0 mL) at 0° C. was added N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide (0.056 g, 0.113 mmol, 1.0 eq) and dimethylaminopyridine (0.001 g, 0.011 mmol, 0.1 eq). Triethylamine (0.023 mL, 0.169 mmol, 1.5 eq) was added and the reaction stirred at 0° C. for 30 minutes and room temperature for 1 hour. The reaction was partitioned between CH₂Cl₂ (30 mL) and NaHCO₃ (30 mL). The aqueous phase was extracted with CH₂Cl₂ (2×30 mL). The combined organics were dried (Na₂SO₄) and concentrated under reduced pressure. MPLC (20→80% acetone-hexane, 0.1% triethylamine) yielded the title compound as a white solid; ¹H nmr (400 MHz, CDCl₃) δ 8.75 (1H, s, 1H of thiazoleH-5, pyrazoleH-5, pyrazoleH-3, H-5), 8.49 (1H, s, 1H of thiazoleH-5, pyrazoleH-5, pyrazoleH-3, H-5), 8.35 (1H, s, 1H of thiazoleH-5, pyrazoleH-5, pyrazoleH-3, H-5), 8.13 (1H, s, 1H of thiazoleH-5, pyrazoleH-5, pyrazoleH-3, H-5), 7.64 (1H, td, J 9.0, 6.0 Hz, pyridineH-4 or H-5), 6.86 (1H, dt, J 8.5, 3.5, 2.5 Hz, pyridineH-4 or H-5), 4.63 (2H, t, J 6.0 Hz, COOCH₂CH₂N), 4.26 (1H, tt, J 11.5, 4.0 Hz, cyclohexaneH-1 or H-4), 3.70, 3.68 (4H, 2d AB system, J 4.5 Hz, 4H of morpholine), 3.55 (2H, q, J 7.0 Hz, OCH₂CH₃), 3.36 (1H, tt, J 10.5, 4.0 Hz, cyclohexaneH-1 or H-4), 2.84 (2H, t, J 6.0 Hz, COOCH₂CH₂N), 2.58, 2.57 (4H, 2d AB system, J 4.5 Hz, 4H of morpholine), 2.28 (2H, m, 2H of cyclohexaneH-2, H-3, H-5, H-6), 2.20 (2H, m, 2H of cyclohexaneH-2, H-3, H-5, H-6), 1.88 (2H, m, 2H of cyclohexaneH-2, H-3, H-5, H-6), 1.45 (2H, m, 2H of cyclohexaneH-2, H-3, H-5, H-6), 1.21 (3H, t, J 7.0 Hz, OCH₂CH₃); ¹⁹F nmr (380 MHz, CDCl₃) δ −72.7 (ddd, J 27.0, 5.5, 4.0 Hz), −124.3 (ddd, 27.0, 11.0, 9.5 Hz); m/z: 657 [M+H]⁺.

### IV. Formation of 3-morpholinopropyl 4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazole-1-carboxylate (VII-15)

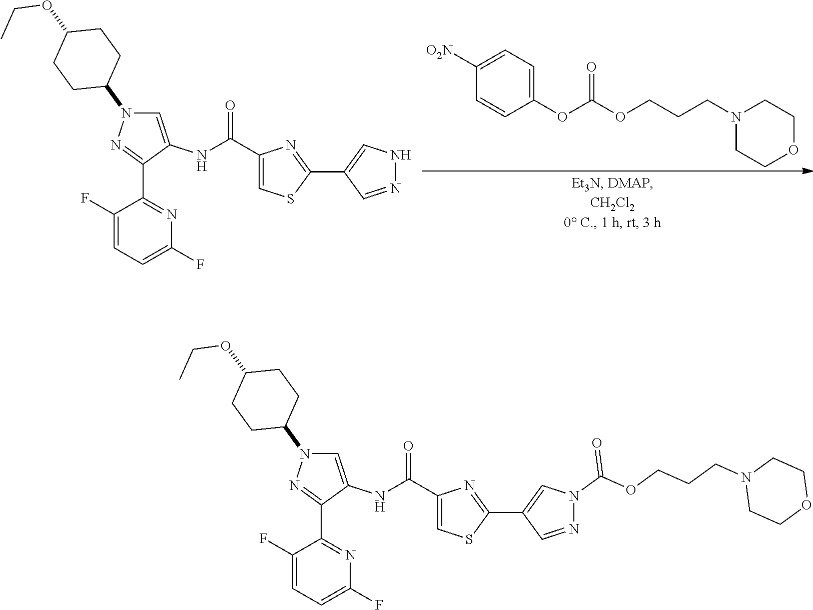

To a mixture of the nitrophenyl carbonate (0.068 g, 0.220 mmol, 1.1 eq) and N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide (0.100 g, 0.200 mmol, 1.0 eq) in dichloromethane (2.0 mL) at 0° C. was added triethylamine (0.031 mL, 0.220 mmol, 1.1 eq) and dimethylaminopyridine (0.002 g, 0.020 mmol, 0.1 eq). The reaction stirred at 0° C. for 1 hour and then at room temperature for 3 hours, resulting an almost clear solution. The reaction was partitioned between CH₂Cl₂ (30 mL) and NaHCO₃ (30 mL). The aqueous phase was extracted with CH₂Cl₂ (2×30 mL). The combined organics were dried (Na₂SO₄) and concentrated under reduced pressure. MPLC (40→100% acetone-hexane, 0.1% triethylamine) yielded the title compound (0.077 g, 57%) as a white solid; ¹H nmr (400 MHz, CDCl₃) δ 8.75 (1H, s, pyrazoleH-5, thiazoleH-5, pyrazoleH-3 or H-5), 8.49 (1H, s, pyrazoleH-5, thiazoleH-5, pyrazoleH-3 or H-5),8.34 (1H, s, pyrazoleH-5, thiazoleH-5, pyrazoleH-3 or H-5), 8.12 (1H, s, pyrazoleH-5, thiazoleH-5, pyrazoleH-3 or H-5),7.64 (1H, td, J 9.0, 6.0 Hz, pyridineH-4 or H-5), 6.87 (1H, ddd, J 9.0, 3.5, 2.5 Hz, pyridineH-4 or H-5), 4.61 (2H, 6.5 Hz, 2H of OCH₂CH₂CH₂N), 4.26 (1H, tt, J 11.5, 4.0 Hz, cyclohexaneH-1 or H-4), 3.66, 3.65 (4H, 2d AB system, J 4.5 Hz, 4H of morpholine), 3.55 (2H, q, J 7.0 Hz, OCH₂CH₃), 3.35 (1H, tt, J 10.5, 4.0 Hz, cyclohexaneH-1 or H-4), 2.52 (2H, J 7.0 Hz, 2H of OCH₂CH₂CH₂N), 2.44 (4H, m, 4H of morpholine), 2.30-2.24 (2H, m, 2H of cyclohexaneH-2, H-3, H-5, H-6), 2.24-2.17 (2H, m, 2H of cyclohexaneH-2, H-3, H-5, H-6), 2.05 (2H, pentet, J 6.5 Hz, OCH₂CH₂CH₂N), 1.93-1.83 (2H, m, 2H of cyclohexaneH-2, H-3, H-5, H-6), 1.51-1.41 (2H, m, 2H of cyclohexaneH-2, H-3, H-5, H-6), 1.21 (3H, t, J 7.0 Hz, OCH₂CH₃); ¹⁹F nmr (380 MHz, CDCl₃) δ −72.7 (ddd, J 28.5, 5.5, 4.0 Hz), −124.3 (ddd, J 28.0, 9.5, 2.5 Hz); m/z: 671 [M+H]⁺ (found [M+H]⁺ 671.2560, C₃₁H₃₆F₂N₈O₅S requires [M+H]⁺ 671.2570).

A person of ordinary skill in the art will understand that the above methods also can be used to make the corresponding urea compounds, such as VII-13 and VII-14, by using an amine in place of the starting hydroxy compound. An exemplary scheme to synthesis urea compound VII-13 is provided below.

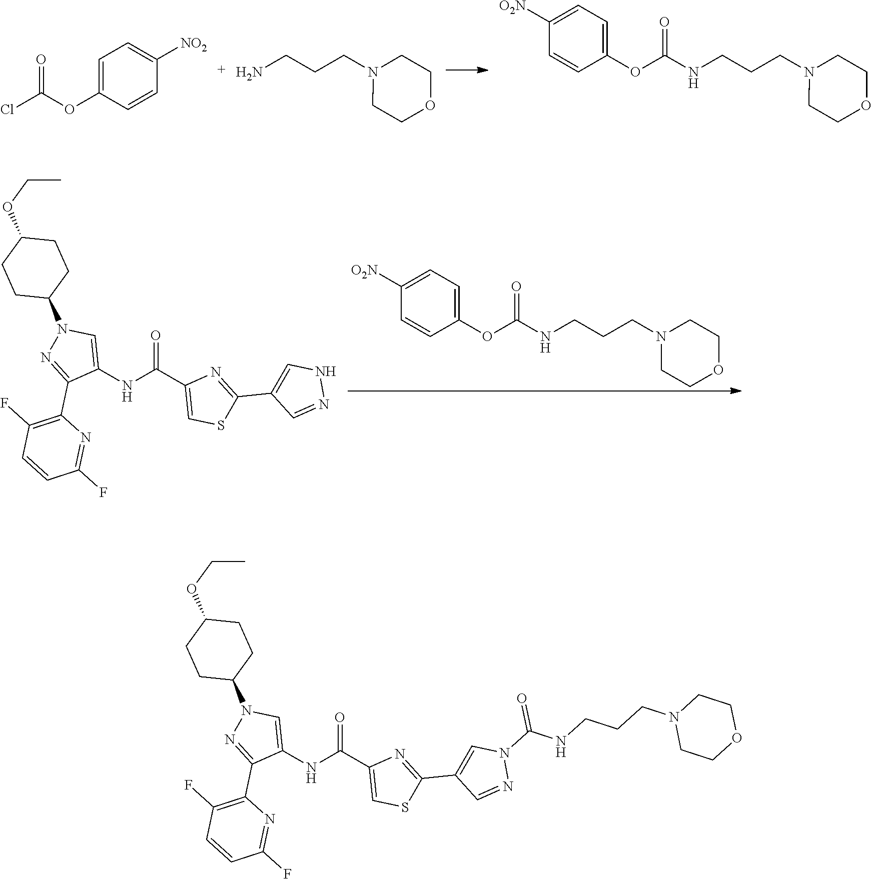

### Exemplary Synthesis of Amino Acid Esters Synthesis of (4-(4-((3-(3,6-difluoropyridin-2-yl)-1-(trans-4-ethoxycyclohexyl)-1H-pyrazol-4-yl)carbamoyl)thiazol-2-yl)-1H-pyrazol-1-yl)methyl L-valinate hydrochloride (VII-16)

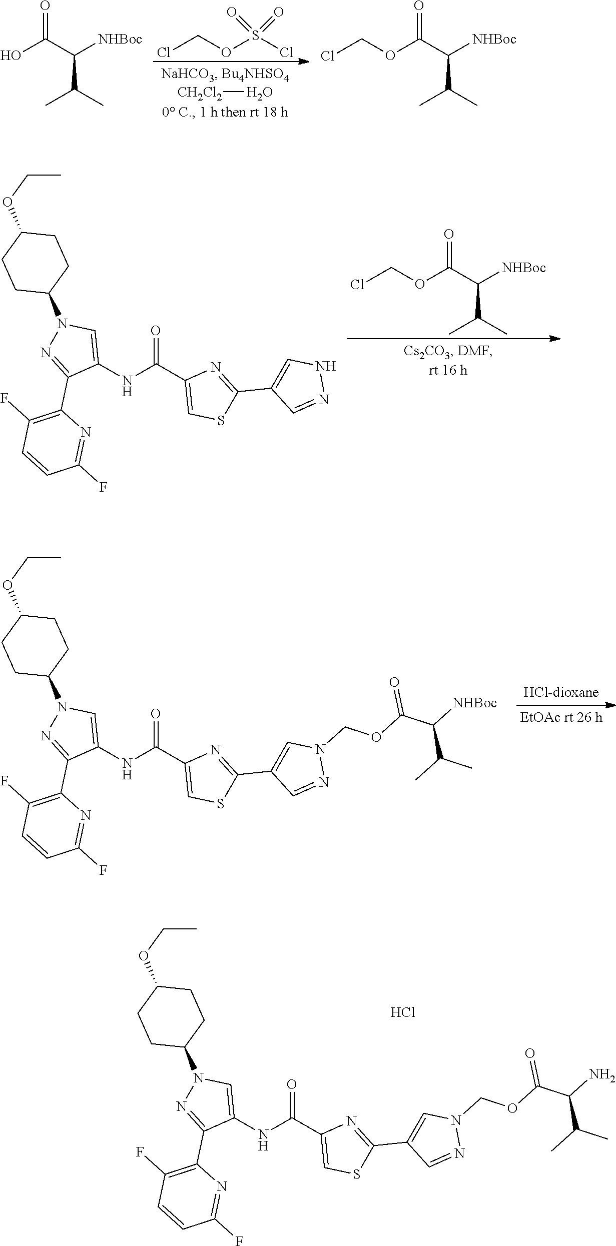

### I. Preparation of chloromethyl (tert-butoxycarbonyl)-L-valinate

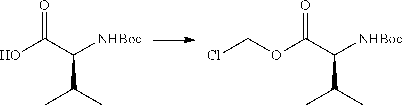 [SMILES: CC(C)[C@H](NC(=O)OC(C)(C)C)C(=O)O ; CC(C)[C@H](NC(=O)OC(C)(C)C)C(=O)OCCl]

To a solution of N-Boc-valine (5.00 g, 23.0 mmol, 1.0 eq) in dichloromethane (100 mL) was added sodium bicarbonate (7.74 g, 92.2 mmol, 4.0 eq) and tetrabutylammonium hydrogen sulfate (0.78 g, 2.3 mmol, 0.1 eq) followed by water (100 mL). The mixture was stirred for 10 minutes to allow for dissolution before cooling to 0° C. and adding a solution of chloromethyl chlorosulfate (3.0 mL, 29.0 mmol, 1.3 eq) in dichloromethane (20 mL) dropwise over 20 minutes. The reaction was stirred at 0° C. for 1 hour and then at room temperature for 18 hours. The reaction was partitioned and the aqueous phase was extracted with CH₂Cl₂ (20 mL). The combined organic phases were washed with water (3×100 mL) and brine (100 mL), dried (Na₂SO₄) and concentrated under reduced pressure to obtain the title compound (6.10 g, quantitative) as a colourless oil; ¹H nmr (400 MHz, CDCl₃) δ 5.87 (1H, d, J 6.0 Hz, 1H of OCH₂Cl), 5.61 (1H, d, J 6.0 Hz, 1H of OCH₂Cl), 4.97 (1H, br d, J 7.0 Hz, NH), 4.27 (1H, dd, J 9.0, 4.5 Hz, COCHNH), 2.22-2.17 (1H, m, CHCH(CH₃)₂), 1.44 (9H, s, C(CH₃)3), 0.99 (3H, d, J 6.5 Hz, 1× CH₃ of CH(CH₃)₂), 0.92 (3H, d, J 7.0 Hz, 1× CH₃ of CH(CH₃)₂).

### II. Preparation of (4-(4-((3-(3,6-difluoropyridin-2-yl)-1-(trans-4-ethoxycyclohexyl)-1H-pyrazol-4-yl)carbamoyl)thiazol-2-yl)-1H-pyrazol-1-yl)methyl (tert-butoxycarbonyl)-L-valinate

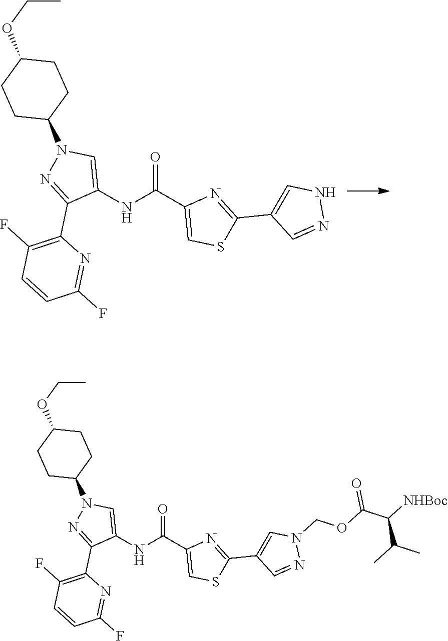 [SMILES: CCO[C@H]1CC[C@H](n2cc(NC(=O)c3csc(-c4cn[nH]c4)n3)c(-c3nc(F)ccc3F)n2)CC1 ; CCO[C@H]1CC[C@H](n2cc(NC(=O)c3csc(-c4cnn(COC(=O)[C@@H](NC(=O)OC(C)(C)C)C(C)C)c4)n3)c(-c3nc(F)ccc3F)n2)CC1]

To a mixture of VII-1 (5.00 g, 10.0 mmol, 1.0 eq) and N-Boc-valine chloromethyl ester (2.93 g, 11.0 mmol, 1.1 eq) was added dimethylformamide (50 mL). Caesium carbonate (3.92 g, 12.0 mmol, 1.2 eq) was added and the reaction stirred at room temperature for 16 hours. The reaction was partitioned between EtOAc (150 mL) and water (150 mL). The organics were washed with brine (100 mL). The combined organics were back-extracted with EtOAc (75 mL). The combined organics were washed with water (200 mL) and brine (150 mL), dried (Na₂SO₄) and concentrated under reduced pressure. MPLC (50→100% EtOAc-hexane) yielded the title compound (6.51 g, 89%) as a white solid; ¹H nmr (400 MHz, CDCl₃) δ 8.48 (1H, s, pyrazoleH-5, thiazoleH-5, pyrazoleH-3 or H-5), 8.29 (1H, s, pyrazoleH-5, thiazoleH-5, pyrazoleH-3 or H-5), 8.14 (1H, s, pyrazoleH-5, thiazoleH-5, pyrazoleH-3 or H-5), 8.04 (1H, s, pyrazoleH-5, thiazoleH-5, pyrazoleH-3 or H-5), 7.63 (1H, td, J 9.0, 6.0 Hz, pyridineH-4 or H-5), 6.87 (1H, ddd, J 9.0, 3.5, 2.5 Hz, pyridineH-4 or H-5), 6.21, 6.02 (2H, 2d AB system, J 10.5 Hz, NCH₂O), 4.94 (1H, d, J 9.0 Hz, NHBoc), 4.28-4.21 (2H, m, cyclohexaneH-1 or H-4, COCHNH), 3.54 (2H, q, J 7.0 Hz, OCH₂CH₃), 3.43 (1H, tt, J 10.5, 4.0 Hz, cyclohexaneH-1 or H-4), 2.30-2.24 (2H, m, 2H of cyclohexaneH-2, H-3, H-5, H-6), 2.23-2.16 (2H, m, 2H of cyclohexaneH-2, H-3, H-5, H-6), 2.13-2.04 (1H, m, CHCH(CH₃)₂), 1.92-1.82 (2H, m, 2H of cyclohexaneH-2, H-3, H-5, H-6), 1.49-1.40 (2H, m, 2H of cyclohexaneH-2, H-3, H-5, H-6), 1.40 (9H, s, C(CH₃)3), 1.20 (3H, t, J 7.0 Hz, OCH₂CH₃), 0.86 (3H, d, J 6.5 Hz, 1× CH₃ of CH(CH₃)₂), 0.77 (3H, d, J 6.5 Hz, 1× CH₃ of CH(CH₃)₂); ¹³C nmr (100 MHz, CDCl₃) δ 171.9, 159.7, 158.2, 15× (d, J 236.5 Hz), 155.6, 153.x (dd, J 260.5, 4.5 Hz), 150.2, 139.8 (d, J 5.0 Hz), 138.9 (t, J 14.5 Hz), 133.0 (d, J 8.5 Hz), 130.5 (d, J 5.0 Hz), 129.9 (dd, J 22.5, 9.0 Hz), 122.0, 121.8, 119.4, 118.6, 107.6 (dd, J 40.5, 5.5 Hz), 80.1, 77.2, 76.4, 72.6, 63.6, 61.5, 58.4, 31.1, 31.0, 30.9, 28.3, 18.8, 17.4, 15.7; ¹⁹F nmr (380 MHz, CDCl₃) δ −72.6, −124.4; m/z: 751 [M+H]⁺, 673 [M+H−C₄H₈]⁺, 629 [M+H−C₄H₈—CO₂]⁺.

### III. Preparation of (4-(4-((3-(3,6-difluoropyridin-2-yl)-1-(trans-4-ethoxycyclohexyl)-1H-pyrazol-4-yl)carbamoyl)thiazol-2-yl)-1H-pyrazol-1-yl)methyl L-valinate hydrochloride, VII-16

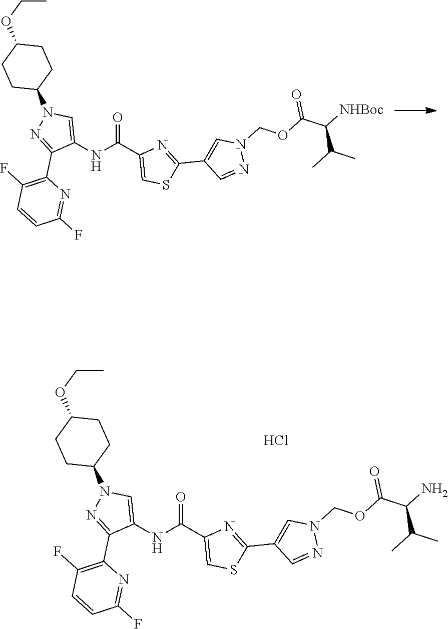 [SMILES: CCO[C@H]1CC[C@H](n2cc(NC(=O)c3csc(-c4cnn(COC(=O)[C@@H](NC(=O)OC(C)(C)C)C(C)C)c4)n3)c(-c3nc(F)ccc3F)n2)CC1 ; CCO[C@H]1CC[C@H](n2cc(NC(=O)c3csc(-c4cnn(COC(=O)[C@@H](N)C(C)C)c4)n3)c(-c3nc(F)ccc3F)n2)CC1 ; Cl]

To a solution/suspension of the Boc-protected valine methylene ester (1.73 g, 2.38 mmol, 1.0 eq) in ethyl acetate (25 mL) was added hydrogen chloride 5.94 mL of a 4M solution in dioxane, 23.76 mmol, 10.0 eq). The reaction was stirred at room temperature for 18 hours. Further hydrogen chloride 3.0 mL of a 4M solution in dioxane, 11.88 mmol, 5.0 eq) was added and the reaction stirred for a further 8 hours before concentrating under reduced pressure. The residue was concentrated from EtOAc (2×30 ml) and dried under vacuum to yield the title compound (1.50 g, quantitative) as a white solid; ¹H nmr (400 MHz, D₆-DMSO) δ 8.66 (1H, s, pyrazoleH-5, thiazoleH-5, pyrazoleH-3 or H-5), 8.51 (1H, s, pyrazoleH-5, thiazoleH-5, pyrazoleH-3 or H-5), 8.35 (1H, s, pyrazoleH-5, thiazoleH-5, pyrazoleH-3 or H-5), 8.22 (1H, s, pyrazoleH-5, thiazoleH-5, pyrazoleH-3 or H-5), 8.07 (1H, td, J 9.0, 6.0 Hz, pyridineH-4 or H-5), 7.25 (1H, ddd, J 8.5, 3.0, 2.5 Hz, pyridineH-4 or H-5), 6.2×, 6.2× (2d, AB system, J Hz, NCH₂OCO), 4.32 (1H, tt, J 11.5, 3.0 Hz, cyclohexaneH-1 or H-4), 3.90 (1H, d, J 4.0 Hz, COCHNH₂), 3.45 (2H, q, J 7.0 Hz, OCH₂CH₃), 3.30 (1H, tt, J 11.0, 4.0 Hz, cyclohexaneH-1 or H-4), 2.12-2.00 (5H, m, 4H of cyclohexaneH-2, H-3, H-5, H-6, CH(CH₃)₂), 1.88-1.80 (2H, m, 2H of cyclohexaneH-2, H-3, H-5, H-6), 1.38-1.29 (2H, m, 2H of cyclohexaneH-2, H-3, H-5, H-6), 1.08 (3H, t, J 7.0 Hz, OCH₂CH₃), 0.87 (3H, d, J 7.0 Hz, 3H of CH(CH₃)₂), 0.83 (3H, d, J 7.0 Hz, 3H of CH(CH₃)₂); ¹⁹F nmr (380 MHz, D6-DMSO) δ −73.0 (d, J 28.5 Hz), −124.1 (dd, J 27.0, 9.5 Hz); m/z: 629 [M+H]⁺ (found [M+H]⁺, 629.2477, C₂₉H₃₄F₂N₈O₄S requires [M+H]⁺ 629.2465).

A person of ordinary skill in the art will understand that this method is generally applicable to any amino acid, particularly a naturally occurring amino acid, as disclosed herein.

### Synthesis of 1-(4-(4-((3-(3,6-difluoropyridin-2-yl)-1-(trans-4-ethoxycyclohexyl)-1H-pyrazol-4-yl)carbamoyl)thiazol-2-yl)-1H-pyrazol-1-yl)ethyl dihydrogen phosphate (VII-18)

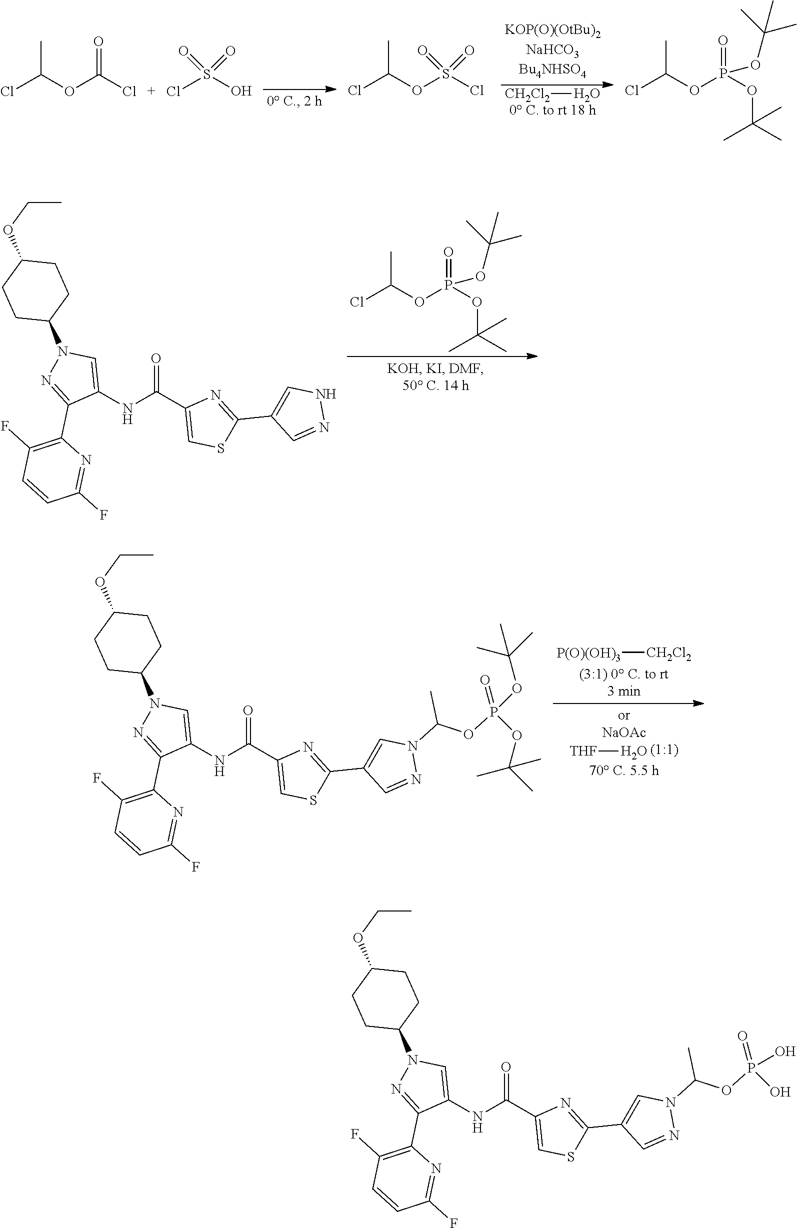

I. Preparation of chloroethyl chlorosulfate

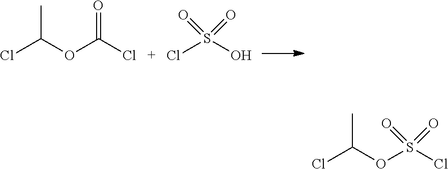

Chlorosulfonic acid (4.90 mL, 73.7 mmol, 1.46 eq) was added dropwise to chloroethyl chloroformate (5.44 mL, 50.4 mmol, 1.0 eq) at 0° C. over 20 minutes. The reaction was stirred at 0° C. for 2 hours and then at room temperature for 10 minutes (during which time the solution temperature rose to 5° C.). Dichloromethane (50 mL) was added followed carefully by ice (2 g), and the mixture stirred rapidly to ensure mixing. Some bubbling was observed and the yellow solution became green-black. The mixture was washed with NaHCO₃ (2×40 mL) to ensure the organics are not acidic. The organics were washed with brine (40 mL), dried (Na₂SO₄) to obtain a clear solution, which was concentrated under reduced pressure to obtain the title compound (4.72 g, 52%) as a black-brown oil; nmr (400 MHz, CDCl₃) δ 6.46 (1H, q, J 6.0 Hz, ClCH(CH₃)O), 1.97 (3H, d, J 5.5 Hz, CHCH₃).

II. Synthesis of 1-chloroethyl di-tert-butyl phosphate

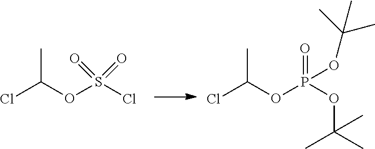

Potassium di-tert-butyl phosphate (5.44 g, 21.97 mmol, 1.0 eq) was dissolved in dichloromethane-water (200 mL, 1:1) and cooled to 0° C. Sodium bicarbonate (7.37 g, 87.74 mmol, 4.0 eq) and tetrabutylammonium hydrogen phosphate (0.74 g, 2.19 mmol, 0.1 eq) were added and the reaction was stirred at 0° C. for 10 minutes. Chloroethyl chlorosulfate (4.72 g as a solution in 20 mL of dichloromethane, 26.37 mmol, 1.2 eq) was then added dropwise over 30 minutes at 0° C. The resulting mixture was stirred rapidly at room temperature for 18 hours and partitioned. The organics were washed with water (3×100 mL) and brine (100 mL), dried (Na₂SO₄) and concentrated under reduced pressure to obtain the title compound (2.35 g, 39%) as a pale brown oil; ¹H nmr (400 MHz, CDCl₃) δ 6.19 (1H, dq, J 8.5, 5.5 Hz, ClCH(CH₃)O), 1.79 (3H, dd, J 5.5, 1.0 Hz, CHCH₃), 1.49 (9H, s, 1× OC(CH₃)₃), 1.48 (9H, s, 1× OC(CH₃)₃); ³²P nmr (380 MHz, CDCl₃) δ −13.0.

III. Preparation of di-tert-butyl (1-(4-(4-((3-(3,6-difluoropyridin-2-yl)-1-(trans-4-ethoxycyclohexyl)-1H-pyrazol-4-yl)carbamoyl)thiazol-2-yl)-1H-pyrazol-1-yl)ethyl) phosphate

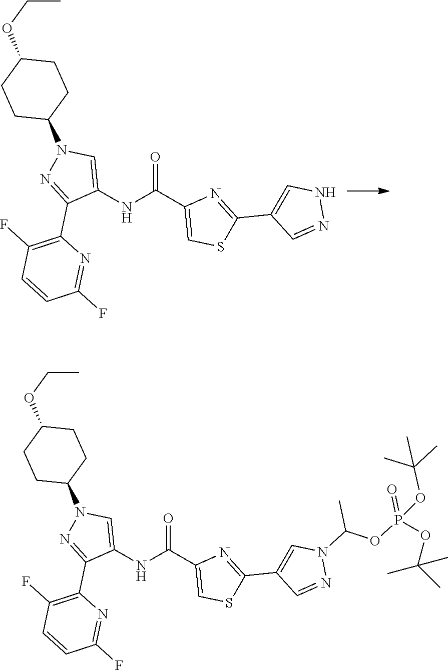 [SMILES: CCO[C@H]1CC[C@H](n2cc(NC(=O)c3csc(-c4cn[nH]c4)n3)c(-c3nc(F)ccc3F)n2)CC1 ; CCO[C@H]1CC[C@H](n2cc(NC(=O)c3csc(-c4cnn(C(C)OP(=O)(OC(C)(C)C)OC(C)(C)C)c4)n3)c(-c3nc(F)ccc3F)n2)CC1]

To a suspension of VII-1 (2.00 g, 4.01 mmol, 1.0 eq) in degassed dimethylformamide (15 mL) was added potassium iodide (0.07 g, 0.40 mmol, 0.1 eq) and potassium hydroxide (0.90 g, 16.03 mmol, 4.0 eq) as small flakes. Chloroethyl di-tert-butyl phosphate (1.64 g as a solution in 5 mL of dimethylformamide, 6.01 mmol, 1.5 eq) was added dropwise over 10 minutes. The resulting mixture was heated to 50° C. for 14 hours before cooling and diluting with EtOAc (50 mL). The reaction was partitioned between EtOAc (100 mL) and water (150 mL). The organics were washed with brine (100 mL), water (150 mL) and brine (100 mL), dried (Na₂SO₄) and concentrated under reduced pressure. Column chromatography (silica, 50→100% EtOAc-hexane) yielded the title compound as a white solid; ¹H nmr (400 MHz, CDCl₃) δ 11.73 (1H, s, NH), 8.51 (1H, s, pyrazoleH-5, thiazoleH-5, pyrazoleH-3 or H-5), 8.33 (1H, s, pyrazoleH-5, thiazoleH-5, pyrazoleH-3 or H-5), 8.16 (1H, s, pyrazoleH-5, thiazoleH-5, pyrazoleH-3 or H-5), 8.05 (1H, s pyrazoleH-5, thiazoleH-5, pyrazoleH-3 or H-5), 7.65 (1H, td, J 9.0, 6.5 Hz, pyridineH-4 or H-5), 6.88 (1H, ddd, J 8.0, 3.0, 2.5 Hz, pyridineH-4 or H-5), 6.39 (1H, dq, J 7.5, 6.5 Hz, NCH(CH₃)O), 4.27 (1H, tt, J 11.5, 3.5 Hz, cyclohexaneH-1 or H-4), 3.56 (2H, q, J 7.0 Hz, OCH₂CH₃), 3.37 (1H, tt, J 10.5, 4.5 Hz, cyclohexaneH-1 or H-4), 2.32-2.26 (2H, m, 2H of cyclohexaneH-2, H-3, H-5, H-6),2.26-1.90 (2H, m, 2H of cyclohexaneH-2, H-3, H-5, H-6), 1.94 (3H, d, J 6.5 Hz, NCH(CH₃)O), 1.93-1.84 (2H, m, 2H of cyclohexaneH-2, H-3, H-5, H-6), 1.52-1.42 (11H, m, 2H of cyclohexaneH-2, H-3, H-5, H-6, 1× C(CH₃)₃), 1.37 (9H, s, 1× C(CH₃)₃), 1.23 (3H, t, J 7.0 Hz, OCH₂CH₃); ¹⁹F nmr (380 MHz, CDCl₃) δ −72.3, −124.5; ³²P nmr (380 MHz, CDCl₃) δ −11.9; m/z: 758 [M+Na]⁺

IV. Preparation of 1-(4-(4-((3-(3,6-difluoropyridin-2-yl)-1-(trans-4-ethoxycyclohexyl)-1H-pyrazol-4-yl)carbamoyl)thiazol-2-yl)-1H-pyrazol-1-yl)ethyl dihydrogen phosphate

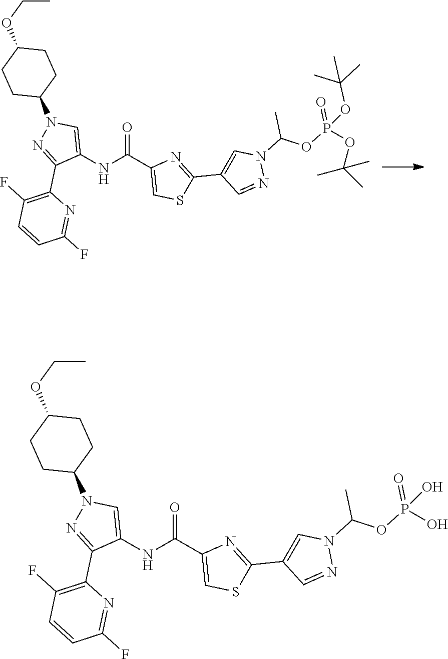 [SMILES: CCO[C@H]1CC[C@H](n2cc(NC(=O)c3csc(-c4cnn(C(C)OP(=O)(OC(C)(C)C)OC(C)(C)C)c4)n3)c(-c3nc(F)ccc3F)n2)CC1 ; CCO[C@H]1CC[C@H](n2cc(NC(=O)c3csc(-c4cnn(C(C)OP(=O)(O)O)c4)n3)c(-c3nc(F)ccc3F)n2)CC1]

A solution of the di-tert-butyl phosphate (0.202 g, 0.275 mmol) in dichloromethane (3 mL) was cooled to 0° C. and phosphoric acid (85%, 9 mL) was added. The reaction was stirred at room temperature for 3 minutes before adding to water (60 mL). The organics were extracted with EtOAc (3×40 mL). The combined organics were dried (Na₂SO₄) and concentrated under reduced pressure to approximately 7 mL. A precipitate formed, which was isolated by filtration to obtain the title compound (0.082 g, 48%) as a pink solid; ¹H nmr (400 MHz, D6-DMSO) δ 11.45 (1H, s, NH), 8.55 (1H, s, pyrazoleH-5, thiazoleH-5, pyrazoleH-3 or H-5), 8.50 (1H, s, pyrazoleH-5, thiazoleH-5, pyrazoleH-3 or H-5), 8.30 (1H, s, pyrazoleH-5, thiazoleH-5, pyrazoleH-3 or H-5), 8.13 (1H, s pyrazoleH-5, thiazoleH-5, pyrazoleH-3 or H-5), 8.06 (1H, td, J 9.5, 6.5 Hz, pyridineH-4 or H-5), 7.24 (1H, dt, J 9.0, 2.5 Hz, pyridineH-4 or H-5), 6.28-6.21 (1H, m, NCH(CH₃)O), 4.31 (1H, br t, J 11.5 Hz, cyclohexaneH-1 or H-4), 3.46 (2H, q, J 7.0 Hz, OCH₂CH₃), 3.30 (1H, br t, J 10.5 Hz, cyclohexaneH-1 or H-4), 2.10-2.03 (4H, m, 4H of cyclohexaneH-2, H-3, H-5, H-6), 1.88-1.78 (2H, m, 2H of cyclohexaneH-2, H-3, H-5, H-6), 1.77 (3H, d, J 6.0 Hz, NCH(CH₃)O), 1.38-1.29 (2H, m, 2H of cyclohexaneH-2, H-3, H-5, H-6), 1.08 (3H, t, J 7.0 Hz, OCH₂CH₃); ¹⁹F nmr (380 MHz, D6-DMSO) δ −72.8, −124.2; ³²P nmr (380 MHz, D6-DMSO) δ −3.3; m/z: 624 [M+H]⁺ (found [M+H]⁺, 624.1610, C₂₅H₂₈F₂N₇O₆PS requires [M+H]⁺ 624.1600).

To a suspension of the di-tert-butyl phosphate (0.100 g, 0.136 mmol, 1.0 eq) in tetrahydrofuran (0.8 mL) water (0.8 mL, distilled, deionized, 18MΩ) was added sodium acetate (0.008 g, 0.010 mmol, 0.75 eq). The reaction was sealed and stirred at 70° C. for 5.5 hours before cooling and adding acetone (20 mL). A precipitate resulted, which was isolated by filtration to obtain the title compound (0.055 g, 65%) as a white solid; data agrees with that stated above.

### Synthesis of (4-(4-((3-(3,6-Difluoropyridin-2-yl)-1-((1r,4r)-4-ethoxycyclohexyl)-1H-pyrazol-4-yl)carbamoyl)thiazol-2-yl)-1H-pyrazol-1-yl)methyl Isopropyl Carbonate (VII-45)

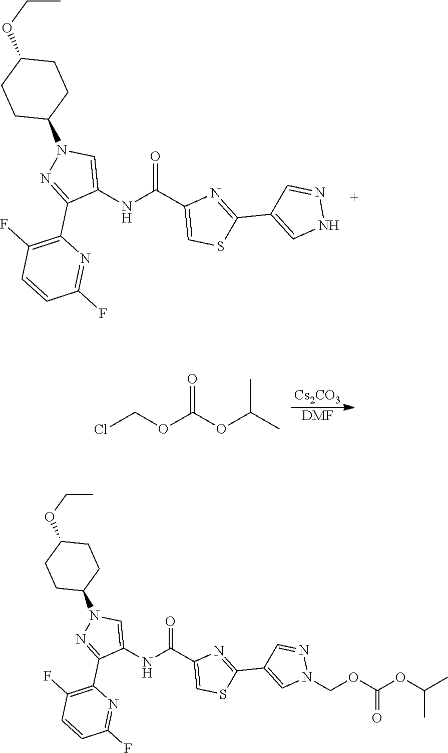

To a solution of N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide (50 mg, 0.1 mmol) and chloromethyl isopropyl carbonate (20 mg, 0.13 mmol) in anhydrous DMF (1 mL) was added cesium carbonate (40 mg, 0.12 mmol). The resulting reaction mixture was then allowed to stir at ambient temperature overnight and then diluted with water (50 mL) to provide upon filtration and drying (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl isopropyl carbonate as a white solid, wt. 49 mg (80%). ¹H NMR (400 MHz, CD₃OD) δ 11.73 (s, 1H), 8.55-8.47 (m, 2H), 8.26-8.15 (m, 2H), 7.88 (ddd, J=9.7, 8.8, 6.2 Hz, 1H), 7.14-7.06 (m, 1H), 6.11 (d, J=4.3 Hz, 2H), 4.96-4.88 (m, 1H), 4.36-4.25 (m, 1H), 3.60 (qd, J=7.0, 1.4 Hz, 2H), 3.52-3.42 (m, 1H), 2.31-2.18 (m, 4H), 1.97 (q, J=11.5 Hz, 2H), 1.54-1.41 (m, 2H), 1.29 (d, J=6.3 Hz, 6H), 1.21 (t, J=7.0 Hz, 3H). MS m/e: Calculated 615.21; Found 616.2 (M+H)⁺.

### Synthesis of (4-(4-((3-(3,6-Difluoropyridin-2-yl)-1-((1r,4r)-4-ethoxycyclohexyl)-1H-pyrazol-4-yl)carbamoyl)thiazol-2-yl)-1H-pyrazol-1-yl)methyl 4-((S)-2-amino-3-methylbutanamido)butanoate Hydrochloride (VII-57)

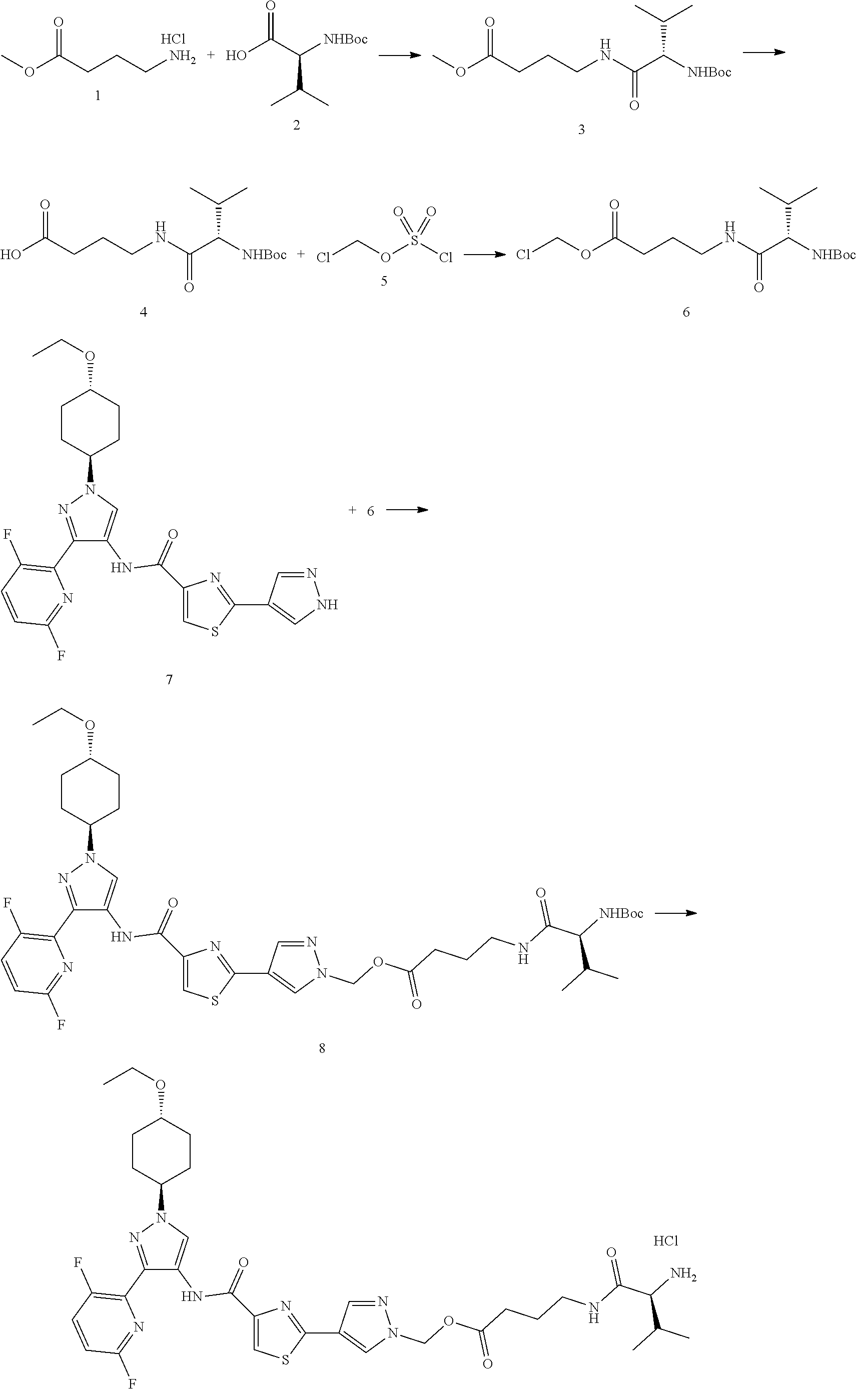

Synthesis of Methyl (S)-4-(2-((tert-Butoxycarbonyl)amino)-3-methylbutanamido)butanoate (3)

To a solution of methyl 4-aminobutanoate hydrogen chloride salt 1 (306 mg, 2.0 mmol) and (tert-butoxycarbonyl)-L-valine 2 (433 mg, 2.0 mmol) in anhydrous DMF (5 mL) was added diisopropylethylamine (568 mg, 0.76 mL, 4.4 mmol). The mixture was then cooled down to 0° C. and HATU (835 mg, 2.2 mmol) was added and the resulting solution was allowed to warm up to ambient temperature and stirred for 17 hours. Water (50 mL) and ethyl acetate (100 mL) were then added and the organic layer was separated, washed with water (3×30 mL), brine (30 mL), dried over anhydrous magnesium sulfate, filtered and concentrated under reduced pressure. The residue obtained was purified by chromatography using 0 to 100% ethyl acetate in hexane gradient to afford methyl (S)-4-(2-((tert-butoxycarbonyl)amino)-3-methylbutanamido)butanoate 3 (59 mg, 94%) as a pale sticky oil. MS m/e: Calculated 316.20; Found 261.1 [M−^{t}Bu+H]⁺.

### II Synthesis of (S)-4-(2-((tert-Butoxycarbonyl)amino)-3-methylbutanamido)butanoic Acid (4)

To a solution of methyl (S)-4-(2-((tert-butoxycarbonyl)amino)-3-methylbutanamido)butanoate 3 (583 mg, 1.85 mmol) in a mixture of THF (4 mL) and MeOH (1 mL) was added NaOH aqueous solution (1 mL, 4 mmol). The resulting solution was stirred at ambient temperature for 15 hours. Most of the solvent mixture was removed under reduced pressure and water (50 mL) was added to the obtained residue. The aqueous layer was then washed with ethyl ether (50 mL), acidified with aqueous HCl (5 mL, 1N) to pH 4 and extracted with ethyl acetate (3×40 mL). Combined organic layer was washed with brine (20 mL), dried over anhydrous magnesium sulfate, filtered and concentrated under reduced pressure to afford (S)-4-(2-((tert-butoxycarbonyl)amino)-3-methylbutanamido)butanoic acid 4 (480 mg, 86%) as a white solid. MS m/e: Calculated 302.18; Found 247.2 [M−^{t}Bu+H]⁺.

III. Synthesis of Chloromethyl (S)-4-(2-((tert-butoxycarbonyl)amino)-3-methylbutanamido)butanoate (6)

To a solution of (S)-4-(2-((tert-butoxycarbonyl)amino)-3-methylbutanamido)butanoic acid 4 (370 mg, 1.23 mmol) in a mixture of dichloromethane (7 mL) and water (7 mL), were added sodium bicarbonate (412 mg, 4.90 mmol) and tetrabutylammonium bisulfate (42 mg, 0.123 mmol), followed by chloromethyl chlorosulfate 5 (233 mg, 143 μL, 1.41 mmol). The resulting solution was stirred at ambient temperature for 2 days and dichloromethane (80 mL) and water (30 mL) were added. The organic layer was separated, and the aqueous layer was extracted with dichloromethane (30 mL). The combined organic layers were dried over anhydrous magnesium sulfate, filtered and concentrated under reduced pressure to afford crude product which was further purified by chromatography using 0 to 100% ethyl acetate in hexane gradient to afford chloromethyl (S)-4-(2-((tert-butoxycarbonyl)amino)-3-methylbutanamido)butanoate 6 (369 mg, 86%) as a colorless oil. MS m/e: Calculated 350.16; Found 251.1 [M−Boc+H]⁺.

IV. Synthesis of (4-(4-((3-(3,6-Difluoropyridin-2-yl)-1-((1r,4r)-4-ethoxycyclohexyl)-1H-pyrazol-4-yl)carbamoyl)thiazol-2-yl)-1H-pyrazol-1-yl)methyl 4-45)-2-((tert-butoxycarbonyl)amino)-3-methylbutanamido)butanoate (8)

To a solution of chloromethyl (S)-4-(2-((tert-butoxycarbonyl)amino)-3-methylbutanamido) butanoate 6 (45 mg, 0.128 mmol) in anhydrous DMF (1 mL) was added diisopropylethylamine (33.2 mg, 45 μL, 0.128 mmol) followed by N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide 7 (64 mg, 0.128 mmol). The resulting solution was stirred at ambient temperature for 2 days, then water (20 mL) was added and the aqueous solution was extracted with ethyl acetate (2×40 mL). The combined organic layers were then washed with brine (20 mL), dried over anhydrous magnesium sulfate, filtered and concentrated under reduced pressure. The resulting crude product was purified by reverse phase HPLC (40 to 100% acetonitrile in water buffered with 0.1% formic acid). Desired fractions were combined and lyophilized to afford (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 4-((S)-2-((tert-butoxycarbonyl)amino)-3-methylbutanamido)butanoate 8 (26 mg, 25%) as a white foam. MS m/e: Calculated 813.34; Found 814.3 [M+H]⁺.

V. Synthesis of (4-(4-((3-(3,6-Difluoropyridin-2-yl)-1-((1r,4r)-4-ethoxycyclohexyl)-1H-pyrazol-4-yl)carbamoyl)thiazol-2-yl)-1H-pyrazol-1-yl)methyl 4-((S)-2-amino-3-methylbutanamido)butanoate Hydrochloride (VII-57)

To a suspension of (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 4-((S)-2-((tert-butoxycarbonyl) amino)-3-methylbutanamido)butanoate 8 (26 mg, 0.032 mmol) in ethyl acetate was added HCl (0.31 mL, 4M in dioxane). The resulting solution was stirred at ambient temperature for 19 hours. A cloudy solution was obtained, filtered and the resulting solid was washed with ethyl acetate and hexanes and dried under high vacuum to afford (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 4-((S)-2-amino-3-methylbutanamido)butanoate hydrogen chloride (21.4 mg, 89%) as a white solid. ¹H NMR (400 MHz, CD₃OD) δ 8.51-8.48 (m, 2H), 8.22 (d, J=0.7 Hz, 1H), 8.20 (s, 1H), 7.89 (td, J=9.2, 6.2 Hz, 1H), 7.09 (ddd, J=8.8, 3.4, 2.6 Hz, 1H), 6.15 (s, 2H), 4.31 (ddd, J=11.7, 8.4, 3.7 Hz, 1H), 3.61 (q, J=7.0 Hz, 2H), 3.53 (d, J=5.9 Hz, 1H), 3.50-3.40 (m, 1H), 3.27 (dt, J=6.9, 3.4 Hz, 2H), 2.48 (t, J=7.4 Hz, 2H), 2.30-2.17 (m, 4H), 2.11 (dq, J=13.4, 6.4 Hz, 1H), 2.05-1.91 (m, 2H), 1.86 (p, J=7.2 Hz, 2H), 1.47 (q, J=11.8 Hz, 2H), 1.21 (t, J=7.0 Hz, 3H), 1.01 (dd, J=6.9, 5.4 Hz, 6H). MS m/e: Calculated 713.29; Found 714.3 [M+H]⁺

### Synthesis of (4-(4-((3-(3,6-Difluoropyridin-2-yl)-1-((1r,4r)-4-ethoxycyclohexyl)-1H-pyrazol-4-yl)carbamoyl)thiazol-2-yl)-1H-pyrazol-1-yl)methyl 1-Amino-3,6,9,12,15,18-hexaoxahenicosan-21-oate Hydrochloride (VII-61)

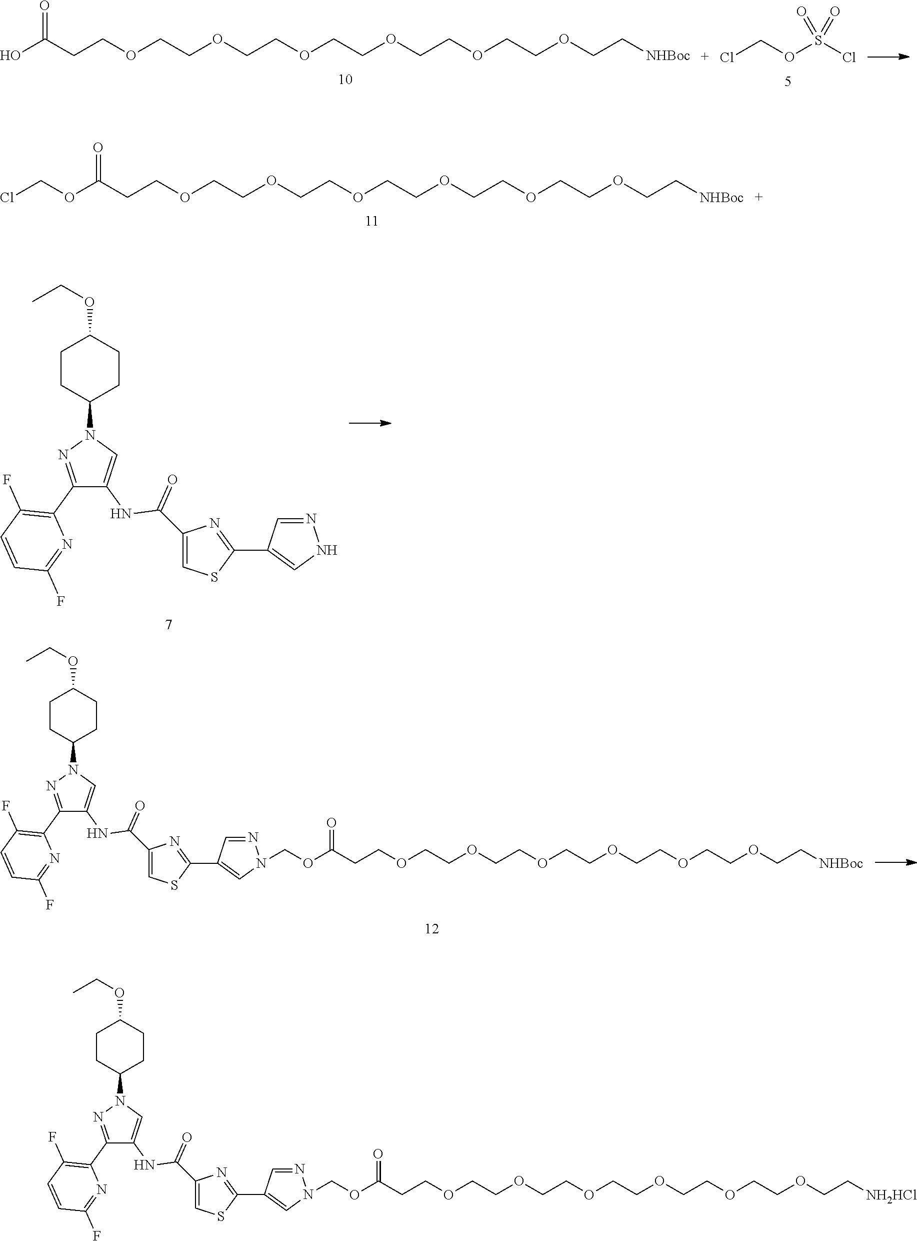

Synthesis of Chloromethyl 2,2-Dimethyl-4-oxo-3,8,11,14,17,20,23-heptaoxa-5-azahexacosan-26-oate (11)

To a solution of 2,2-dimethyl-4-oxo-3,8,11,14,17,20,23-heptaoxa-5-azahexacosan-26-oic acid (250 mg, 0.551 mmol) 10 in the mixture of dichloromethane (5.2 mL) and water (5.2 mL) were added sodium bicarbonate (185 mg, 2.21 mmol) and tetrabutylammonium bisulfate (18.7 mg, 0.0551 mmol). Chloromethyl chlorosulfate 5 (105 mg, 64 4, 0.634 mmol) was then added and the resulting solution was stirred at ambient temperature for 18 hours. Water (10 mL) was then added, and the resulting aqueous solution was extracted with dichloromethane (3×30 mL). The combined organic layers were washed with brine (20 mL), dried over anhydrous magnesium sulfate, filtered and concentrated under reduced pressure to afford crude product of chloromethyl 2,2-dimethyl-4-oxo-3,8,11,14,17,20,23-heptaoxa-5-azahexacosan-26-oate 11 (303 mg, 100%) with 91% purity. The crude product was directly used in next step without further purification. MS m/e: Calculated 501.23; Found 402.1 [M−Boc+H]⁺.

II. Synthesis of (4-(4-((3-(3,6-Difluoropyridin-2-yl)-1-((1r,4r)-4-ethoxycyclohexyl)-1H-pyrazol-4-yl)carbamoyl)thiazol-2-yl)-1H-pyrazol-1-yl)methyl 2,2-dimethyl-4-oxo-3,8,11,14,17,20,23-heptaoxa-5-azahexacosan-26-oate (12)

To a solution of chloromethyl 2,2-dimethyl-4-oxo-3,8,11,14,17,20,23-heptaoxa-5-azahexacosan-26-oate 11 (51.8 mg, 0.103 mmol) and N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide 7 (51.5 mg, 0.103 mmol) in anhydrous DMF (1 mL) was added anhydrous cesium carbonate (37 mg, 0.113 mmol). The resulting reaction mixture was stirred at ambient temperature for 16 hours. Water (20 mL) and ethyl acetate (100 mL) were then added, and the organic layer was separated, washed with brine, dried over anhydrous magnesium sulfate, filtered and concentrated under reduced pressure. The residue obtained was purified by reverse phase HPLC (30 to 100% acetonitrile in water buffered with 0.1% formic acid). The desired fractions were combined, lyophilized to afford (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 2,2-dimethyl-4-oxo-3,8,11,14,17,20,23-heptaoxa-5-azahexacosan-26-oate 12 (57.4 mg, 58%) as a colorless sticky oil. MS m/e: Calculated 964.42; Found 865.3[M−Boc+H]⁺.

III. Synthesis of (4-(4-((3-(3,6-Difluoropyridin-2-yl)-1-((1r,4r)-4-ethoxycyclohexyl)-1H-pyrazol-4-yl)carbamoyl)thiazol-2-yl)-1H-pyrazol-1-yl)methyl 1-Amino-3,6,9,12,15,18-hexaoxahenicosan-21-oate Hydrochloride (VII-61)

To a solution of (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 2,2-dimethyl-4-oxo-3,8,11,14,17,20,23-heptaoxa-5-azahexacosan-26-oate 12 (57.4 mg, 0.0595 mmol) in ethyl acetate (5 mL) was added HCl (2.4 mL, 1M in ethyl ether, 2.4 mmol). The resulting solution was stirred at ambient temperature for 2 days. All solvents were removed under reduced pressure and the residue obtained was purified by reverse phase HPLC (0 to 70% acetonitrile in water buffered with 0.1% formic acid). The desired fractions were combined and HCl solution (65 4, 1N) was added and lyophilized to afford (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 1-amino-3,6,9,12,15,18-hexaoxahenicosan-21-oate hydrochloride (19 mg, 35%) as a sticky pale yellow solid. ¹H NMR (400 MHz, CD₃OD) δ 11.71 (s, 1H), 8.50 (s, 2H), 8.28-8.16 (m, 2H), 7.90 (td, J=9.2, 6.1 Hz, 1H), 7.21-7.00 (m, 1H), 6.17 (s, 2H), 4.31 (ddd, J=11.8, 8.3, 3.7 Hz, 1H), 3.76 (t, J=5.9 Hz, 2H), 3.72-3.48 (m, 24H), 3.06 (t, J=5.1 Hz, 2H), 2.70 (t, J=5.9 Hz, 2H), 2.66 (s, 1H), 2.30-2.17 (m, 4H), 1.97 (dt, J=13.7, 11.2 Hz, 2H), 1.56-1.41 (m, 2H), 1.29 (s, 3H), 1.21 (t, J=7.0 Hz, 3H). MS m/e: Calculated 864.37; Found 865.3 [M+H]⁺.

### Synthesis of Isopropyl (((4-(4-((3-(3,6-Difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)(phenoxy)phosphoryl)-L-alaninate (VII-62)

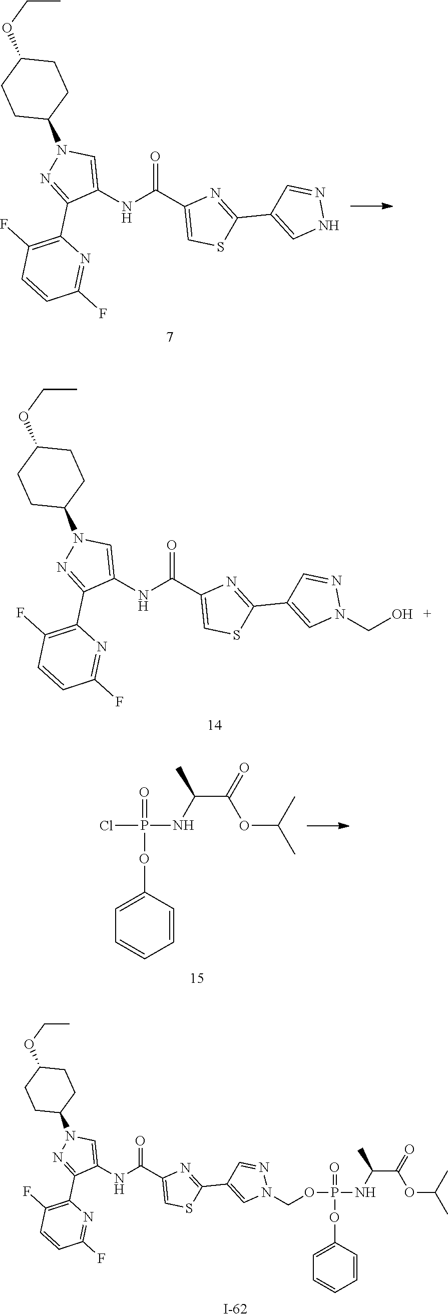

Synthesis of N-(3-(3,6-Difluoropyridin-2-yl)-1-((1r,4r)-4-ethoxycyclohexyl)-1H-pyrazol-4-yl)-2-(1-(hydroxymethyl)-1H-pyrazol-4-yl)thiazole-4-carboxamide (14)

To a solution of N-(3-(3 ,6-difluoropyridin-2-yl)-1-((1r,4r)-4-ethoxycyclohexyl)-1H-pyrazol-4-yl)-2-(1H-pyrazol-4-yl)thiazole-4-carboxamide 7 (501 mg, 1 mmol) in absolute ethanol (3 mL) was added formaldehyde aqueous solution (162 mg, 0.15 mL, 37% wt., 2 mmol). The resulting solution was heated at 50° C. for 18 hours, and the resulting cloudy reaction mixture was filtered, washed with absolute ethanol and hexanes. The white solid obtained was placed under high vacuum to afford N-(3-(3,6-difluoropyridin-2-yl)-1-((1r,4r)-4-ethoxycyclohexyl)-1H-pyrazol-4-yl)-2-(1-(hydroxymethyl)-1H-pyrazol-4-yl)thiazole-4-carboxamide 14 (385 mg, 73%). ¹H NMR (400 MHz, DMSO-d₆) δ 11.47 (s, 1H), 8.52 (d, J=8.5 Hz, 2H), 8.31 (s, 1H), 8.10 (d, J=15.2 Hz, 2H), 7.28 (s, 1H), 6.99 (s, 1H), 5.43 (d, J=7.7 Hz, 2H), 4.33 (s, 1H), 3.47 (d, J=7.4 Hz, 2H), 2.08 (d, J=11.9 Hz, 4H), 1.86 (d, J=13.4 Hz, 2H), 1.35 (d, J=12.3 Hz, 2H), 1.10 (t, J=7.0 Hz, 3H). MS m/e: Calculated 529.17; Found 530.1[M+H]⁺.

II. Synthesis of Isopropyl (((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)(phenoxy)phosphoryl)-L-alaninate (VII-62)

To a solution of N-(3-(3,6-difluoropyridin-2-yl)-1-((1r,4r)-4-ethoxycyclohexyl)-1H-pyrazol-4-yl)-2-(1-(hydroxymethyl)-1H-pyrazol-4-yl)thiazole-4-carboxamide 14 (57.3 mg, 0.108 mmol) in anhydrous dichloromethane (2 mL), diisopropylethylamine (28 mg, 38 4, 0.217 mmol) was added followed by isopropyl (chloro(phenoxy)phosphoryl)-L-alaninate 15 (36.4 mg, 30 4, 0.119 mmol). The resulting solution was stirred at ambient temperature for 2 days and then concentrated under reduced pressure. The residue obtained was purified by reverse phase HPLC (50 to 100% acetonitrile in water buffered with 0.1% formic acid) and the desired fractions were combined and lyophilized to afford isopropyl (((4-(4-((3-(3,6-difluoropyridin-2-yl)-1-((1r, 4r)-4-ethoxycyclohexyl)-1H-pyrazol-4-yl)carbamoyl)thiazol-2-yl)-1H-pyrazol-1-yl)methoxy)(phenoxy)phosphoryl)-L-alaninate (16 mg, 19%) as a white solid. ¹H NMR (400 MHz, CD₃OD) δ 8.51 (s, 1H), 8.48 (d, J=14.4 Hz, 1H), 8.24 (d, J=4.5 Hz, 1H), 8.22 (s, 1H), 7.87 (ddd, J=9.7, 8.8, 6.2 Hz, 1H), 7.33-7.25 (m, 2H), 7.21-7.01 (m, 4H), 6.11 (d, J=11.8 Hz, 1H), 6.06 (dd, J=11.6, 2.3 Hz, 1H), 4.95 (pd, J=6.3, 5.3 Hz, 1H), 4.38-4.25 (m, 1H), 3.99-3.81 (m, 1H), 3.60 (q, J=7.0 Hz, 2H), 3.51-3.39 (m, 1H), 2.32-2.14 (m, 4H), 1.98 (q, J=12.1, 11.6 Hz, 2H), 1.47 (q, J=12.1 Hz, 2H), 1.32 (ddd, J=8.8, 7.2, 1.2 Hz, 3H), 1.26-1.09 (m, 9H). MS m/e: Calculated 798.25; Found 799.2 [M+H]⁺

### Synthesis of ((((4-(4-(3-(3,6-Difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)(hydroxy)phosphorypoxy)methyl isopropyl carbonate (VII-60)

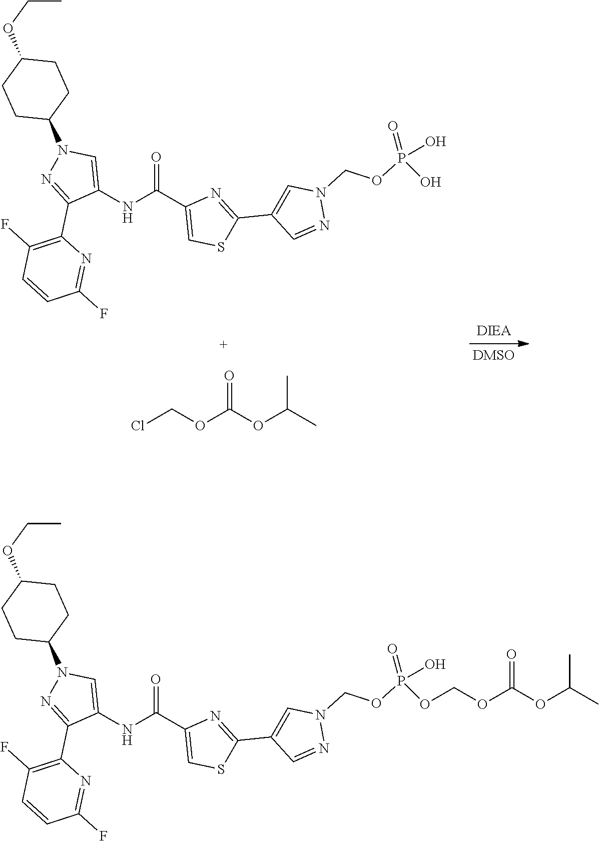

To a solution of (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl dihydrogen phosphate (1.00 g, 1.64 mmol, 1.0 eq) in dimethyl sulfoxide (10 mL) was added chloromethyl isopropyl carbonate (2.17 mL, 16.4 mmol, 10 eq) and diisopropylethylamine (2.71 mL, 16.4 mmol, 10 eq). The solution was stirred at room temperature for 2 days. The reaction mixture was purified by reverse phase HPLC (C-18, water/acetonitrile with 0.1% formic acid) to give the title compound (309 mg, 26%) as a white solid. ¹H NMR (400 MHz, CDCl₃) δ 11.6 (s, 1H), 8.37 (s, 1H), 8.25 (s, 1H), 8.03 (s, 1H), 7.95 (s, 1H), 7.57-7.51 (m, 1H), 6.81-6.79 (m, 1H), 5.97 (d, J=10.8 Hz, 2H), 5.65 (d, J=10.8 Hz, 2H), 4.93-4.87 (m, 1H), 4.27-4.21 (m, 1H), 3.57 (q, J=7.2, 6.8 Hz, 2H), 3.41-3.35 (m, 1H), 2.32-2.22 (m, 4H), 1.93-1.84 (m, 2H), 1.52-1.43 (m, 2H), 1.33-1.24 (m, 9H). MS m/e: Calculated 725.18; Found 726.2 (M+H)⁺.

The following exemplary compounds were prepared using the methods of above. Characterization data for these additional compounds are provided below.

### VII-6: 2-(1-(acetyl-L-leucyl)-1H-pyrazol-4-yl)-N-(3-(3,6-difluoropyridin-2-yl)-1-((1r,4r)-4-ethoxycyclohexyl)-1H-pyrazol-4-yl)thiazole-4-carboxamide

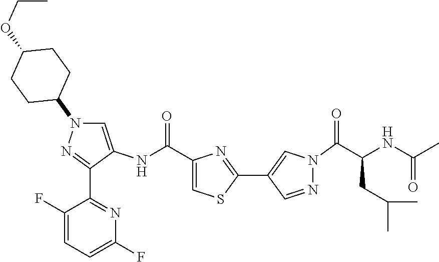

¹H nmr (400 MHz, CDCl₃) δ 8.78 (1H, s, pyrazoleH-3 or H-5), 8.50 (1H, s, thiazoleH-5 or pyrazoleH-5), 8.36 (1H, s, pyrazoleH-3 or H-5), 8.14 (1H, s, thiazoleH-5 or pyrazoleH-5), 7.65 (1H, td, J 9.0, 6.0 Hz, pyridineH-4 or H-5), 6.91 (1H, ddd, J 9.0, 3.5, 2.5 Hz, pyridineH-4 or H-5), 6.11 (1H, d, J 9.0 Hz, NHCOCH₃), 5.88 (1H, m, COCHNHCO), 4.27 (1H, tt, J 11.5, 4.0 Hz, cyclohexaneH-1 or H-4), 3.56 (2H, q, J 7.0 Hz, OCH₂CH₃), 3.37 (1H, tt, J 10.5, 4.0 Hz, cyclohexaneH-1 or H-4), 2.30 (2H, m, 2H of cyclohexaneH-2, H-3, H-5, H-6), 2.22 (2H, m, 2H of cyclohexaneH-2, H-3, H-5, H-6), 2.08 (3H, s, COCH₃), 1.89 (2H, m, 2H of cyclohexaneH-2, H-3, H-5, H-6), 1.86-1.76 (2H, m, 2H of CHCH₂CH(CH₃)₂), 1.65 (1H, m, 1H of CHCH₂CH(CH₃)₂), 1.33 (2H, m, 2H of cyclohexaneH-2, H-3, H-5, H-6), 1.22 (3H, t, J 7.0 Hz, OCH₂CH₃), 1.07 (3H, d, J 6.0 Hz, 1× CH₃ of CH(CH₃)₂), 0.97 (3H, d, J 6.5 Hz, 1× CH₃ of CH(CH₃)₂); m/z: 677 [M+Na]⁺, 655 [M+H]⁺ (found [M+H]⁺, 655.2623, C₃₁H₃₆F₂N₈O₄S requires [M+H]⁺655.2621).

### VII-7: 1-methylcyclopropyl 4-(44(3-(3,6-difluoropyridin-2-yl)-1-((1r,4r)-4-ethoxycyclohexyl)-1H-pyrazol-4-yl)carbamoyl)thiazol-2-yl)-1H-pyrazole-1-carboxylate

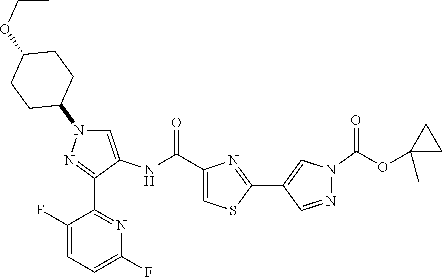

¹H nmr (400 MHz, CDCl₆) δ 8.73 (1H, s, 1H of thiazoleH-5, pyrazoleH-5 or pyrazoleH-3, H-5), 8.50 (1H, s, 1H of thiazoleH-5, pyrazoleH-5 or pyrazoleH-3, H-5), 8.33 (1H, s, 1H of thiazoleH-5, pyrazoleH-5 or pyrazoleH-3, H-5), 8.13 (1H, s, 1H of thiazoleH-5, pyrazoleH-5 or pyrazoleH-3, H-5), 7.66 (1H, td, J 9.0, 6.0 Hz, pyridineH-4 or H-5), 6.88 (1H, ddd, J 9.0, 3.5, 2.5 Hz, pyridineH-4 or H-5), 4.28 (1H, tt, J 11.5, 4.0 Hz, cyclohexaneH-1 or H-4), 3.56 (2H, q, J 7.0 Hz, OCH₂CH₃), 3.37 (1H, tt, J 10.5, 4.0 Hz, cyclohexaneH-1 or H-4), 2.30 (2H, br t, J 11.5 Hz, 2H of cyclohexaneH-2, H-3, H-5, H-6), 2.22 (2H, m, 2H of cyclohexaneH-2, H-3, H-5, H-6), 1.89 (2H, m, 2H of cyclohexaneH-2, H-3, H-5, H-6), 1.76 (3H, s, CH₃), 1.47 (2H, m, 2H of cyclohexaneH-2, H-3, H-5, H-6), 1.24 (2H, m, 2H of cPrH-2, H-3), 1.23 (3H, t, J 7.0 Hz, OCH₂CH₃), 0.86 (2H, m, 2H of cPrH-2, H-3); ¹⁹F nmr (380 MHz, CDCl₃) δ −72.6, −124.3; m/z: 598 [M+H]⁺ (found [M+H]⁺, 598.2035, C₂₈H₂₉F₂N₇O₄S requires [M+H]⁺598.2043).

### VII-8: 1-(isobutyryloxy)ethyl 4-(44(3-(3,6-difluoropyridin-2-yl)-1-((1r,4r)-4-ethoxycyclohexyl)-1H-pyrazol-4-yl)carbamoyl)thiazol-2-yl)-1H-pyrazole-1-carboxylate

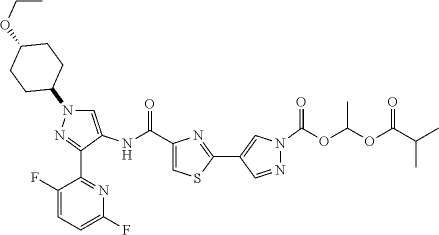

¹H nmr (400 MHz, CDCl₃) δ 8.76 (1H, s, 1H of thiazoleH-5, pyrazoleH-5, pyrazoleH-3, H-5), 8.51 (1H, s, 1H of thiazoleH-5, pyrazoleH-5, pyrazoleH-3, H-5), 8.38 (1H, s, 1H of thiazoleH-5, pyrazoleH-5, pyrazoleH-3, H-5), 8.14 (1H, s, 1H of thiazoleH-5, pyrazoleH-5, pyrazoleH-3, H-5), 7.66 (1H, td, J 9.0, 6.0 Hz, pyridineH-4 or H-5), 7.15 (1H, q, J 5.5 Hz, OCH(CH₃)O), 6.87 (1H, ddd, J 9.0, 3.5, 2.5 Hz, pyridineH-4 or H-5), 4.28 (1H, tt, J 11.5, 4.0 Hz, cyclohexaneH-1 or H-4), 3.57 (2H, q, J 7.0 Hz, OCH₂CH₃), 3.37 (1H, tt, J 10.5, 4.0 Hz, cyclohexaneH-1 or H-4), 2.63 (1H, heptet, J 7.0 Hz, COCH(CH₃)₂), 2.30 (2H, m, 2H of cyclohexaneH-2, H-3, H-5, H-6), 2.22 (2H, m, 2H of cyclohexaneH-2, H-3, H-5, H-6), 1.90 (2H, m, 2H of cyclohexaneH-2, H-3, H-5, H-6), 1.74 (3H, d, J 5.5 Hz, OCH(CH₃)O), 1.47 (2H, m, 2H of cyclohexaneH-2, H-3, H-5, H-6), 1.23 (3H, t, J 7.0 Hz, OCH₂CH₃), 1.21 (3H, d, J 7.0 Hz, 1× CH₃ of (CH(CH₃)₂), 1.21 (3H, d, J 6.5 Hz, 1× CH₃ of CH(CH₃)₂); ¹⁹F nmr (380 MHz, CDCl₃) δ −72.6 (ddd, J 27.0, 5.5, 4.0 Hz), −124.3 (ddd, 27.0, 9.5, 2.5 Hz); m/z: 658 [M+H]⁺ (found [M+H]⁺, 658.2553, C₃₀H₃₃F₂N₇O₆S requires [M+H]⁺658.2254).

### VII-9: N-(3-(3,6-difluoropyridin-2-yl)-1-((1r,4r)-4-ethoxycyclohexyl)-1H-pyrazol-4-yl)-2-(1-((5-methyl-2-oxo-1,3-dioxol-4-yl)methyl)-1H-pyrazol-4-yl)thiazole-4-carboxamide

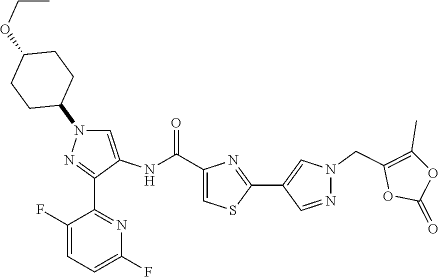

¹H nmr (400 MHz, CDCl₃) δ 8.50 (1H, s, 1H of thiazoleH-5, pyrazoleH-5, pyrazoleH-3, H-5), 8.49 (1H, s, 1H of thiazoleH-5, pyrazoleH-5, pyrazoleH-3, H-5), 8.11 (1H, s, 1H of thiazoleH-5, pyrazoleH-5, pyrazoleH-3, H-5), 8.09 (1H, s, 1H of thiazoleH-5, pyrazoleH-5, pyrazoleH-3, H-5), 7.67 (1H, td, J 9.0, 6.5 Hz, pyridineH-4 or H-5), 6.92 (1H, dt, J 9.0, 3.0 Hz, pyridineH-4 or H-5), 5.19 (1H, d, J 4.5 Hz, 1H of NCH₂C), 4.73 (1H, d, J 4.5 Hz, 1H of NCH₂C), 4.28 (1H, tt, J 11.5, 4.0 Hz, cyclohexaneH-1 or H-4), 3.57 (2H, q, J 7.0 Hz, OCH₂CH₃), 3.38 (1H, tt, J 10.5, 4.0 Hz, cyclohexaneH-1 or H-4), 2.36 (3H, s, CCH₃), 2.30 (2H, m, 2H of cyclohexaneH-2, H-3, H-5, H-6), 2.23 (2H, m, 2H of cyclohexaneH-2, H-3, H-5, H-6), 1.90 (2H, m, 2H of cyclohexaneH-2, H-3, H-5, H-6), 1.48 (2H, m, 2H of cyclohexaneH-2, H-3, H-5, H-6), 1.23 (3H, t, J 7.0 Hz, OCH₂CH₃); ¹⁹F nmr (380 MHz, CDCl₃) δ −73.5, −124.1 (ddd, 27.0, 9.5, 3.0 Hz); m/z: 612 [M+H]⁺ (found [M+H]⁺, 612.1835, C₂₈H₂₇F₂N₇O₅S requires [M+H]⁺612.1857).

### VII-10: 2-morpholinoethyl 4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazole-1-carboxylate

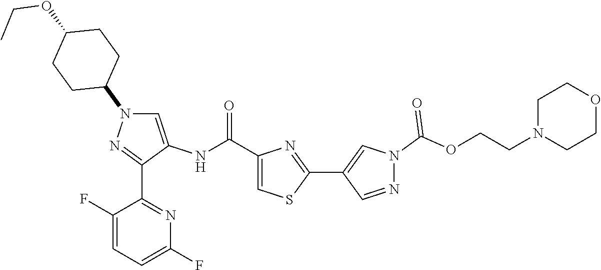

¹H nmr (400 MHz, CDCl₃) δ 8.75 (1H, s, 1H of thiazoleH-5, pyrazoleH-5, pyrazoleH-3, H-5), 8.49 (1H, s, 1H of thiazoleH-5, pyrazoleH-5, pyrazoleH-3, H-5), 8.35 (1H, s, 1H of thiazoleH-5, pyrazoleH-5, pyrazoleH-3, H-5), 8.13 (1H, s, 1H of thiazoleH-5, pyrazoleH-5, pyrazoleH-3, H-5), 7.64 (1H, td, J 9.0, 6.0 Hz, pyridineH-4 or H-5), 6.86 (1H, dt, J 8.5, 3.5, 2.5 Hz, pyridineH-4 or H-5), 4.63 (2H, t, J 6.0 Hz, COOCH₂CH₂N), 4.26 (1H, tt, J 11.5, 4.0 Hz, cyclohexaneH-1 or H-4), 3.70, 3.68 (4H, 2d AB system, J 4.5 Hz, 4H of morpholine), 3.55 (2H, q, J 7.0 Hz, OCH₂CH₃), 3.36 (1H, tt, J 10.5, 4.0Hz, cyclohexaneH-1 or H-4), 2.84 (2H, t, J 6.0 Hz, COOCH₂CH₂N), 2.58, 2.57 (4H, 2d AB system, J 4.5 Hz, 4H of morpholine), 2.28 (2H, m, 2H of cyclohexaneH-2, H-3, H-5, H-6), 2.20 (2H, m, 2H of cyclohexaneH-2, H-3, H-5, H-6), 1.88 (2H, m, 2H of cyclohexaneH-2, H-3, H-5, H-6), 1.45 (2H, m, 2H of cyclohexaneH-2, H-3, H-5, H-6), 1.21 (3H, t, J 7.0 Hz, OCH₂CH₃); ¹⁹F nmr (380 MHz, CDCl₃) δ −72.7 (ddd, J 27.0, 5.5, 4.0 Hz), −124.3 (ddd, 27.0, 11.0, 9.5 Hz); m/z: 657 [M+H]⁺.

### VII-12: N-(3-(3,6-difluoropyridin-2-yl)-1-((1r,4r)-4-ethoxycyclohexyl)-1H-pyrazol-4-yl)-2-(1-(morpholine-4-carbonyl)-1H-pyrazol-4-yl)thiazole-4-carboxamide

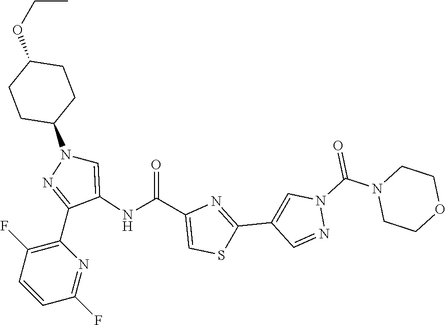 [SMILES: CCO[C@H]1CC[C@H](n2cc(NC(=O)c3csc(-c4cnn(C(=O)N5CCOCC5)c4)n3)c(-c3nc(F)ccc3F)n2)CC1]

1H nmr (400 MHz, CDCl₃) δ 8.71 (1H, s, pyrazoleH-5, thiazoleH-5, pyrazoleH-3 or H-5), 8.50 (1H, s, pyrazoleH-5, thiazoleH-5, pyrazoleH-3 or H-5), 8.26 (1H, d, J 0.5 Hz,), 8.10 (1H, s, pyrazoleH-5, thiazoleH-5, pyrazoleH-3 or H-5), 7.64 (1H, td, J 9.0, 6.0 Hz, pyridineH-4 or H-5), 6.90 (1H, ddd, J 9.0, 3.5, 2.5 Hz, pyridineH-4 or H-5), 4.27 (1H, tt, J 11.5, 4.0 3.83, 3.82 (4H, 2d AB system, J 4.0 Hz, 4H of morpholine), 3.56 (2H, q, J 7.0 Hz, OCH₂CH₃), 3.36 (1H, tt, J 11.0, 4.0 Hz, cyclohexaneH-1 or H-4), Hz, cyclohexaneH-1 or H-4), 3.94 (4H, br s, 4H of morpholine), 2.33-2.25 (2H, m, 2H of cyclohexaneH-2, H-3, H-5, H-6), 2.55-1.90 (2H, m, 2H of cyclohexaneH-2, H-3, H-5, H-6), 1.94-1.84 (2H, m, 2H of cyclohexaneH-2, H-3, H-5, H-6), 1.52-1.41 (2H, m, 2H of cyclohexaneH-2, H-3, H-5, H-6), 1.22 (3H, t, J 7.0 Hz, OCH₂CH₃); ¹⁹F nmr (380 MHz, CDCl₃) δ −72.5, −124.4; m/z: 613 [M+H]⁺ (found [M+H]⁺, 613.2163, C₂₈H₃₀F₂N₈O₄S requires [M+H]⁺613.2152).

### VII-13: N-(3-(3,6-difluoropyridin-2-yl)-1-((1r,4r)-4-ethoxycyclohexyl)-1H-pyrazol-4-yl)-2-(1-((3-morpholinopropyl)carbamoyl)-1H-pyrazol-4-yl)thiazole-4-carboxamide

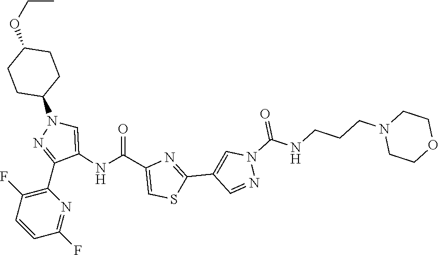 [SMILES: CCO[C@H]1CC[C@H](n2cc(NC(=O)c3csc(-c4cnn(C(=O)NCCCN5CCOCC5)c4)n3)c(-c3nc(F)ccc3F)n2)CC1]

¹H nmr (400 MHz, CDCl₃) δ 8.85 (1H, t, J 5.0 Hz, CONHCH₂), 8.79 (1H, s, pyrazoleH-5, thiazoleH-5, pyrazoleH-3 or H-5), 8.49 (1H, s, pyrazoleH-5, thiazoleH-5, pyrazoleH-3 or H-5), 8.25 (1H, s, pyrazoleH-5, thiazoleH-5, pyrazoleH-3 or H-5), 8.08 (1H, s, pyrazoleH-5, thiazoleH-5, pyrazoleH-3 or H-5), 7.36 (1H, td, J 9.0, 6.0 Hz, pyridineH-4 or H-5), 6.90 (1H, ddd, J 9.0, 3.5, 2.5 Hz, pyridineH-4 or H-5), 4.26 (1H, tt, J 12.0, 4.0 Hz, cyclohexaneH-1 or H-4), 3.85, 3.84 (4H, 2d AB system, J 4.5 Hz, 4H of morpholine), 3.60-3.56 (2H, m, CONHCH₂CH₂CH₂N), 3.55 (2H, q, J 7.0 Hz, OCH₂CH₃), 3.36 (1H, tt, J 10.5, 4.0 Hz, cyclohexaneH-1 or H-4), 2.57-2.54 (2H, m, CONHCH₂CH₂CH₂N), 2.51 (4H, br s, 4H of morpholine), 2.30-2.26 (2H, m, 2H of cyclohexaneH-2, H-3, H-5, H-6), 2.23-2.18 (2H, m, 2H of cyclohexaneH-2, H-3, H-5, H-6), 1.93-1.84 (2H, m, 2H of cyclohexaneH-2, H-3, H-5, H-6), 1.84-1.78 (2H, m, CONHCH₂CH₂CH₂N), 1.51-1.41 (2H, m, 2H of cyclohexaneH-2, H-3, H-5, H-6), 1.21 (3H, t, J 7.0 Hz, OCH₂CH₃); ¹⁹F nmr (380 MHz, CDCl₃) δ −72.6 (ddd, J 27.0, 5.5, 4.0 Hz), −124.5 (ddd, J 27.0, 9.5, 2.5 Hz); m/z: 670 [M+H]⁺.

### VII-14: N-(3-(3,6-difluoropyridin-2-yl)-1-((1r,4r)-4-ethoxycyclohexyl)-1H-pyrazol-4-yl)-2-(1-((3-(dimethylamino)propyl)carbamoyl)-1H-pyrazol-4-yl)thiazole-4-carboxamide

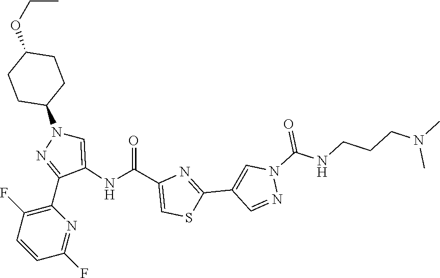 [SMILES: CCO[C@H]1CC[C@H](n2cc(NC(=O)c3csc(-c4cnn(C(=O)NCCCN(C)C)c4)n3)c(-c3nc(F)ccc3F)n2)CC1]

¹H nmr (400 MHz, CDCl₃) δ 8.80 (1H, s, pyrazoleH-5, thiazoleH-5, pyrazoleH-3 or H-5), 8.49 (1H, s pyrazoleH-5, thiazoleH-5, pyrazoleH-3 or H-5), 8.36 (1H, t, J 5.5 Hz, pyrazoleCONH), 8.20 (1H, d, J 0.5 Hz, pyrazoleH-5, thiazoleH-5, pyrazoleH-3 or H-5), 8.08 (1H, s, pyrazoleH-5, thiazoleH-5, pyrazoleH-3 or H-5), 7.63 (1H, td, J 9.0, 6.0 Hz, pyridineH-4 or H-5), 6.89 (1H, ddd, J 9.0, 3.5, 2.5 Hz, pyridineH-4 or H-5), 4.26 (1H, tt, J 11.5, 4.0 Hz, cyclohexaneH-1 or H-4), 3.58-3.52 (4H, m, OCH₂CH₃, pyrazoleCONHCH₂), 3.36 (1H, tt, J 10.5, 4.0 Hz, cyclohexaneH-1 or H-4), 2.44 (2H, t, J 6.5 Hz, CH₂N(CH₃)₂), 2.26 (6H, s, N(CH₃)₂), 2.30-2.18 (4H, m, 4H of cyclohexaneH-2, H-3, H-5, H-6), 1.93-1.83 (2H, m, 2H of cyclohexaneH-2, H-3, H-5, H-6), 1.79 (2H, pentet, J 6.5 Hz, NCH₂CH₂CH₂N(CH₃)₂), 1.51-1.41 (2H, m, 2H of cyclohexaneH-2, H-3, H-5, H-6), 1.21 (3H, t, J 7.0 Hz, OCH₂CH₃); ¹⁹F nmr (380 MHz, CDCl₃) δ −72.6, −124.5; m/z: 628 [M+H]⁺ (found [M+H]⁺, 628.2628, C₂₉H₃₅F₂N₉O₃S requires [M+H]⁺628.2624).

### VII-15: 3-morpholinopropyl 4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazole-1-carboxylate

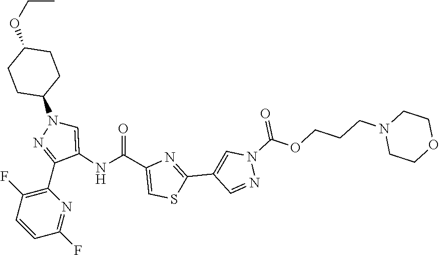 [SMILES: CCO[C@H]1CC[C@H](n2cc(NC(=O)c3csc(-c4cnn(C(=O)OCCCN5CCOCC5)c4)n3)c(-c3nc(F)ccc3F)n2)CC1]

¹H nmr (400 MHz, CDCl₃) δ 8.75 (1H, s, pyrazoleH-5, thiazoleH-5, pyrazoleH-3 or H-5), 8.49 (1H, s, pyrazoleH-5, thiazoleH-5, pyrazoleH-3 or H-5),8.34 (1H, s, pyrazoleH-5, thiazoleH-5, pyrazoleH-3 or H-5), 8.12 (1H, s, pyrazoleH-5, thiazoleH-5, pyrazoleH-3 or H-5),7.64 (1H, td, J 9.0, 6.0 Hz, pyridineH-4 or H-5), 6.87 (1H, ddd, J 9.0, 3.5, 2.5 Hz, pyridineH-4 or H-5), 4.61 (2H, 6.5 Hz, 2H of OCH₂CH₂CH₂N), 4.26 (1H, tt, J 11.5, 4.0 Hz, cyclohexaneH-1 or H-4), 3.66, 3.65 (4H, 2d AB system, J 4.5 Hz, 4H of morpholine), 3.55 (2H, q, J 7.0 Hz, OCH₂CH₃), 3.35 (1H, tt, J 10.5, 4.0 Hz, cyclohexaneH-1 or H-4), 2.52 (2H, J 7.0 Hz, 2H of OCH₂CH₂CH₂N), 2.44 (4H, m, 4H of morpholine), 2.30-2.24 (2H, m, 2H of cyclohexaneH-2, H-3, H-5, H-6), 2.24-2.17 (2H, m, 2H of cyclohexaneH-2, H-3, H-5, H-6), 2.05 (2H, pentet, J 6.5 Hz, OCH₂CH₂CH₂N), 1.93-1.83 (2H, m, 2H of cyclohexaneH-2, H-3, H-5, H-6), 1.51-1.41 (2H, m, 2H of cyclohexaneH-2, H-3, H-5, H-6), 1.21 (3H, t, J 7.0 Hz, OCH₂CH₃); ¹⁹F nmr (380 MHz, CDCl₃) δ −72.7 (ddd, J 28.5, 5.5, 4.0 Hz), −124.3 (ddd, J 28.0, 9.5, 2.5 Hz); m/z: 671 [M+H]⁺ (found [M+H]⁺, 671.2560, C₃₁H₃₆F₂N₈O₅S requires [M+H]⁺ 671.2570).

### VII-16: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl L-valinate hydrogen chloride salt

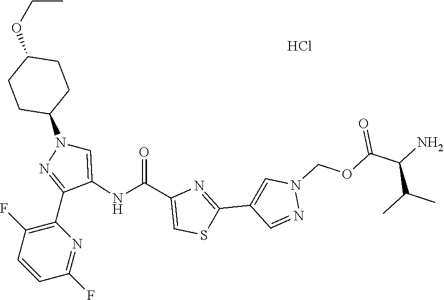 [SMILES: CCO[C@H]1CC[C@H](n2cc(NC(=O)c3csc(-c4cnn(COC(=O)[C@@H](N)C(C)C)c4)n3)c(-c3nc(F)ccc3F)n2)CC1 ; Cl]

¹H nmr (400 MHz, D₆-DMSO) δ 8.66 (1H, s, pyrazoleH-5, thiazoleH-5, pyrazoleH-3 or H-5), 8.51 (1H, s, pyrazoleH-5, thiazoleH-5, pyrazoleH-3 or H-5), 8.35 (1H, s, pyrazoleH-5, thiazoleH-5, pyrazoleH-3 or H-5), 8.22 (1H, s, pyrazoleH-5, thiazoleH-5, pyrazoleH-3 or H-5), 8.07 (1H, td, J 9.0, 6.0 Hz, pyridineH-4 or H-5), 7.25 (1H, ddd, J 8.5, 3.0, 2.5 Hz, pyridineH-4 or H-5), 6.2x , 6.2x (2d, AB system, J Hz, NCH₂OCO), 4.32 (1H, tt, J 11.5, 3.0 Hz, cyclohexaneH-1 or H-4), 3.90 (1H, d, J 4.0 Hz, COCHNH₂), 3.45 (2H, q, J 7.0 Hz, OCH₂CH₃), 3.30 (1H, tt, J 11.0, 4.0 Hz, cyclohexaneH-1 or H-4), 2.12-2.00 (5H, m, 4H of cyclohexaneH-2, H-3, H-5, H-6, CH(CH₃)₂), 1.88-1.80 (2H, m, 2H of cyclohexaneH-2, H-3, H-5, H-6), 1.38-1.29 (2H, m, 2H of cyclohexaneH-2, H-3, H-5, H-6), 1.08 (3H, t, J 7.0 Hz, OCH₂CH₃), 0.87 (3H, d, J 7.0 Hz, 3H of CH(CH₃)₂), 0.83 (3H, d, J 7.0 Hz, 3H of CH(CH₃)₂); ¹³C nmr (100 MHz, D₆-DMSO) δ 168.8, 160.2, 157.6, 157.5 (d, J 236.0 Hz), 153.5 (dd, J 259.0, 4.5 Hz), 149.4, 139.5 (d, 6.5 Hz), 138.2 (t, J 14.5 Hz), 132.6 (d, 8.5 Hz), 132.3, 131.9 (dd, 22.5, 9.5 Hz), 124.4, 121.4, 120.3, 117.8, 109.2 (br d, J 34.0 Hz), 76.0, 73.6, 63.0, 60.8, 57.4, 30.9 (2C), 29.8, 18.6, 17.7, 16.1; ¹⁹F nmr (380 MHz, D₆-DMSO) δ −73.0 (d, J 28.5 Hz), −124.1 (dd, J 27.0, 9.5 Hz); m/z: 629 [M+H]⁺ (found [M+H]⁺, 629.2477, C₂₉H₃₄F₂N₈O₄S requires [M+H]⁺ 629.2465).

### VII-17: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl L-prolinate hydrogen chloride salt

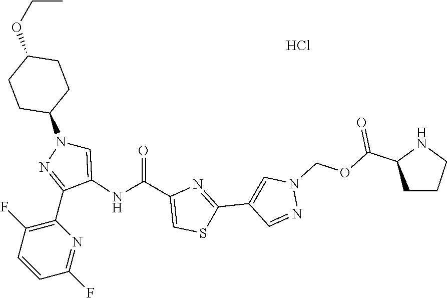 [SMILES: CCO[C@H]1CC[C@H](n2cc(NC(=O)c3csc(-c4cnn(COC(=O)[C@@H]5CCCN5)c4)n3)c(-c3nc(F)ccc3F)n2)CC1 ; Cl]

1H nmr (400 MHz, D₆-DMSO) δ 11.48 (1H, s, 1× NH), 9.32 (1H, br s, 1× NH), 8.66 (1H, pyrazoleH-5, thiazoleH-5, pyrazoleH-3 or H-5), 8.51 (1H, s, pyrazoleH-5, thiazoleH-5, pyrazoleH-3 or H-5), 8.35 (1H, s, pyrazoleH-5, thiazoleH-5, pyrazoleH-3 or H-5), 8.22 (1H, s, pyrazoleH-5, thiazoleH-5, pyrazoleH-3 or H-5), 8.07 (1H, td, J 9.5, 6.5 Hz, pyridineH-4 or H-5), 7.26 (1H, dt, J 8.5, 2.5 Hz, pyridineH-4 or H-5), 6.24 (2H, s, NCH₂OCOCHN), 4,42 (1H, tt, J 8.5, 3.5 Hz, cyclohexaneH-1 or H-4), 3.45 (2H, q, J 7.0 Hz, OCH₂CH₃), 3.33 (1H, tt, J 10.0, 4.0 Hz, cyclohexaneH-1 or H-4), 3.23-3.11 (2H, m, COCHNHCH₂), 2.27-2.19 (1H, m, 1H of COCH(NH)CH₂), 2.10-2.04 (4H, m, 4H of cyclohexaneH-2, H-3, H-5, H-6), 1.98-1.80 (5H, m, 2H of cyclohexaneH-2, H-3, H-5, H-6, 3H of COCH(NH)CH₂CH₂), 1.38-1.29 (2H, m, 2H of cyclohexaneH-2, H-3, H-5, H-6), 1.08 (3H, t, J 7.0 Hz, OCH₂CH₃); ¹⁹F nmr (380 MHz, D6-DMSO) δ −73.0 (d, J 27.5 Hz), −124.1 (dd, J 27.0, 9.5 Hz); m/z: 627 [M+H]⁺.

### VII-18: 1-(4-(44(3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl dihydrogen phosphate

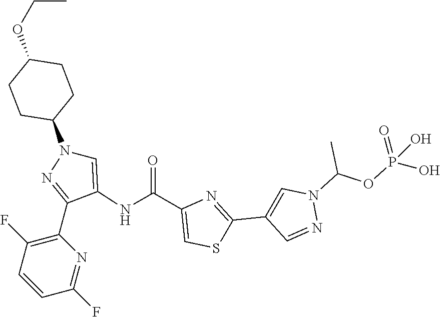 [SMILES: CCO[C@H]1CC[C@H](n2cc(NC(=O)c3csc(-c4cnn(C(C)OP(=O)(O)O)c4)n3)c(-c3nc(F)ccc3F)n2)CC1]

1H nmr (400 MHz, D6-DMSO) δ 11.45 (1H, s, NH), 8.55 (1H, s, pyrazoleH-5, thiazoleH-5, pyrazoleH-3 or H-5), 8.50 (1H, s, pyrazoleH-5, thiazoleH-5, pyrazoleH-3 or H-5), 8.30 (1H, s, pyrazoleH-5, thiazoleH-5, pyrazoleH-3 or H-5), 8.13 (1H, s pyrazoleH-5, thiazoleH-5, pyrazoleH-3 or H-5), 8.06 (1H, td, J 9.5, 6.5 Hz, pyridineH-4 or H-5), 7.24 (1H, dt, J 9.0, 2.5 Hz, pyridineH-4 or H-5), 6.28-6.21 (1H, m, NCH(CH₃)O), 4.31 (1H, br t, J 11.5 Hz, cyclohexaneH-1 or H-4), 3.46 (2H, q, J 7.0 Hz, OCH₂CH₃), 3.30 (1H, br t, J 10.5 Hz, cyclohexaneH-1 or H-4), 2.10-2.03 (4H, m, 4H of cyclohexaneH-2, H-3, H-5, H-6), 1.88-1.78 (2H, m, 2H of cyclohexaneH-2, H-3, H-5, H-6), 1.77 (3H, d, J 6.0 Hz, NCH(CH₃)O), 1.38-1.29 (2H, m, 2H of cyclohexaneH-2, H-3, H-5, H-6), 1.08 (3H, t, J 7.0 Hz, OCH₂CH₃); ¹⁹F nmr (380 MHz, D6-DMSO) δ −72.8, −124.2; ³²P nmr (380 MHz, D6-DMSO) δ −3.3; m/z: 624 [M+H]⁺ (found [M+H]⁺, 624.1610, C₂₅H₂₈F₂N₇O₆PS requires [M+H]⁺ 624.1600).

### VII-19: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl glycinate hydrogen chloride salt

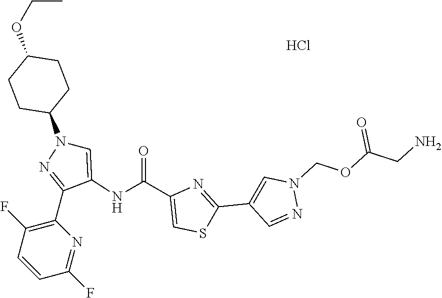 [SMILES: CCO[C@H]1CC[C@H](n2cc(NC(=O)c3csc(-c4cnn(COC(=O)CN)c4)n3)c(-c3nc(F)ccc3F)n2)CC1 ; Cl]

¹H nmr (400 MHz, D6-DMSO) δ 11.47 (1H, s, NH), 8.67 (1H, s, 1H of pyrazoleH-5, thiazoleH-5, pyrazoleH-3 or H-5), 8.52 (1H, s, 1H of pyrazoleH-5, thiazoleH-5, pyrazoleH-3 or H-5), 8.37 (1H, s, 1H of pyrazoleH-5, thiazoleH-5, pyrazoleH-3 or H-5), 8.34 (2H, br s, NH₂), 8.23 (1H, s, 1H of pyrazoleH-5, thiazoleH-5, pyrazoleH-3 or H-5), 8.09 (1H, td, J 9.5, 6.5 Hz, pyridineH-4 or H-5), 7.27 (1H, dt, J 8.5, 2.5 Hz, pyridineH-4 or H-5), 6.25 (2H, s, NCH₂O or COCH₂NH₂), 4.33 (1H, tt, J 11.5, 3.5 Hz, cyclohexaneH-1 or H-4), 3.89 (2H, s, NCH₂O or COCH₂NH₂), 3.47 (2H, q, J 7.0 Hz, OCH₂CH₃), 3.34 (1H, tt, J 11.0, 3.5 Hz, cyclohexaneH-1 or H-4), 2.12-2.04 (4H, m, 4H of cyclohexaneH-2, H-3, H-5, H-6), 1.91-1.80 (2H, m, 2H of cyclohexaneH-2, H-3, H-5, H-6), 1.41-1.29 (2H, m, 2H of cyclohexaneH-2, H-3, H-5, H-6), 1.10 (3H, t, J 7.0 Hz, OCH₂CH₃); ¹⁹F nmr (380 MHz, D6-DMSO) δ −72.9, −124.1; m/z: 587 [M+H]⁺ (found [M+H]⁺, 587.1996, C₂₆H₂₈F₂N₈O₄S requires [M+H]⁺ 587.1995).

### VII-20: sodium 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate

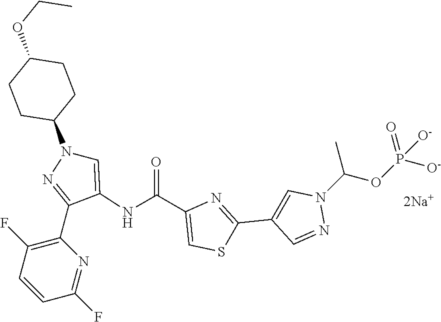 [SMILES: CCO[C@H]1CC[C@H](n2cc(NC(=O)c3csc(-c4cnn(C(C)OP(=O)([O-])[O-])c4)n3)c(-c3nc(F)ccc3F)n2)CC1]

¹H nmr (400 MHz, D₂O) δ 8.05 (1H, s, pyrazoleH-5, thiazoleH-5, pyrazoleH-3 or H-5), 7.86 (1H, s, pyrazoleH-5, thiazoleH-5, pyrazoleH-3 or H-5), 7.55 (1H, s, pyrazoleH-5, thiazoleH-5, pyrazoleH-3 or H-5), 7.52 (1H, s pyrazoleH-5, thiazoleH-5, pyrazoleH-3 or H-5), 7.37 (1H, m, pyridineH-4 or H-5), 6.59 (1H, m, pyridineH-4 or H-5), 6.00 (1H, t, J 7.5 Hz, NCH(CH₃)O), 3.94 (1H, m, cyclohexaneH-1 or H-4), 3.56 (2H, q, J 7.0 Hz, OCH₂CH₃), 3.43 (1H, m, cyclohexaneH-1 or H-4), 2.16-2.08 (2H, m, 2H of cyclohexaneH-2, H-3, H-5, H-6), 2.07-2.00 (2H, m, 2H of cyclohexaneH-2, H-3, H-5, H-6), 1.69 (3H, d, J 6.0 Hz, NCH(CH₃)O), 1.68-1.60 (2H, m, 2H of cyclohexaneH-2, H-3, H-5, H-6), 1.36-1.25 (2H, m, 2H of cyclohexaneH-2, H-3, H-5, H-6), 1.10 (3H, t, J 7.0 Hz, OCH₂CH₃); ¹⁹F nmr (380 MHz, D₂O) 6-72.8, −124.8; ³²P nmr (380 MHz, D₂O) δ 1.2; m/z: 624 [M+H]⁺.

### VII-21: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl (S)-2-amino-3,3-dimethylbutanoate hydrogen chloride salt

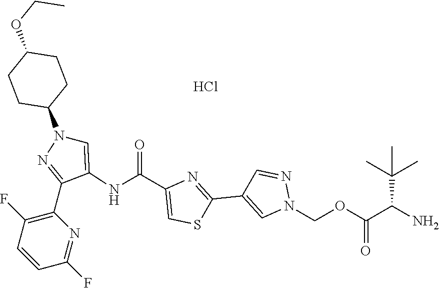 [SMILES: CCO[C@H]1CC[C@H](n2cc(NC(=O)c3csc(-c4cnn(COC(=O)[C@@H](N)C(C)(C)C)c4)n3)c(-c3nc(F)ccc3F)n2)CC1 ; Cl]

¹H nmr (400 MHz, D6-DMSO) δ 11.47 (1H, s, NH), 8.68 (1H, s, 1H of pyrazoleH-5, thiazoleH-5, pyrazoleH-3 or H-5), 8.52 (1H, s, 1H of pyrazoleH-5, thiazoleH-5, pyrazoleH-3 or H-5), 8.43 (2H, br s, NH₂), 8.37 (1H, s, 1H of pyrazoleH-5, thiazoleH-5, pyrazoleH-3 or H-5), 8.24 (1H, s, 1H of pyrazoleH-5, thiazoleH-5, pyrazoleH-3 or H-5), 8.09 (1H, td, J 9.5, 6.5 Hz, pyridineH-4 or H-5), 7.26 (1H, br d, J 8.5 Hz, pyridineH-4 or H-5), 6.34, 6.24 (2H, 2d AB system, J 11.0 Hz, NCH₂O), 4.33 (1H, br t, J 11.5, Hz, cyclohexaneH-1 or H-4), 3.86 (1H, s, COCH(tBu)NH₂), 3.47 (2H, q, J 7.0 Hz, OCH₂CH₃), 3.38-3.30 (1H, m, cyclohexaneH-1 or H-4), 2.12-2.05 (4H, m, 4H of cyclohexaneH-2, H-3, H-5, H-6), 1.91-1.81 (2H, m, 2H of cyclohexaneH-2, H-3, H-5, H-6), 1.40-1.30 (2H, m, 2H of cyclohexaneH-2, H-3, H-5, H-6), 1.10 (3H, t, J 7.0 Hz, OCH₂CH₃), 0.93 (9H, s, C(CH₃)3); ¹⁹F nmr (380 MHz, D6-DMSO) δ −72.9, −124.1; m/z: 643 [M+H]⁺ (found [M+H]⁺, 643.2607, C₃₀H₃₆F₂N₈O₄S requires [M+H]⁺ 643.2621).

### VII-23: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 2-amino-2-methylpropanoate hydrogen chloride salt

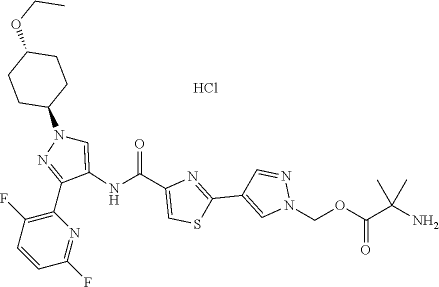 [SMILES: CCO[C@H]1CC[C@H](n2cc(NC(=O)c3csc(-c4cnn(COC(=O)C(C)(C)N)c4)n3)c(-c3nc(F)ccc3F)n2)CC1 ; Cl]

¹H nmr (400 MHz, D6-DMSO) δ 8.68 (1H, s, 1H of pyrazoleH-5, thiazoleH-5, pyrazoleH-3 or H-5), 8.52 (2H, br s, 2× NH), 8.52 (1H, s, 1H of pyrazoleH-5, thiazoleH-5, pyrazoleH-3 or H-5), 8.37 (1H, s, 1H of pyrazoleH-5, thiazoleH-5, pyrazoleH-3 or H-5), 8.24 (1H, s, 1H of pyrazoleH-5, thiazoleH-5, pyrazoleH-3 or H-5), 8.09 (1H, td, J 9.0, 6.5 Hz, pyridineH-4 or H-5), 7.26 (1H, dt, J 9.0, 3.0 Hz, pyridineH-4 or H-5), 6.26 (2H, s, NCH₂O), 4.33 (1H, br t, J 12.0 Hz, cyclohexaneH-1 or H-4), 3.47 (2H, q, J 7.0 Hz, OCH₂CH₃), 3.34 (1H, tt, J 10.5, 3.5 Hz, cyclohexaneH-1 or H-4), 2.11-2.04 (4H, m, 4H of cyclohexaneH-2, H-3, H-5, H-6), 1.91-1.80 (2H, m, 2H of cyclohexaneH-2, H-3, H-5, H-6), 1.43 (6H, s, C(CH₃)₂), 1.41-1.30 (2H, m, 2H of cyclohexaneH-2, H-3, H-5, H-6), 1.10 (3H, t, J 7.0 Hz, OCH₂CH₃); ¹⁹F nmr (380 MHz, D6-DMSO) δ −72.9, −124.1; m/z: 615 [M+H]⁺ (found [M+H]⁺, 615.2343, C₂₈H₃₂F₂N₈O₄S requires [M+H]⁺ 615.2309).

### VII-24: 4-(14-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)-4-oxobutanoic acid

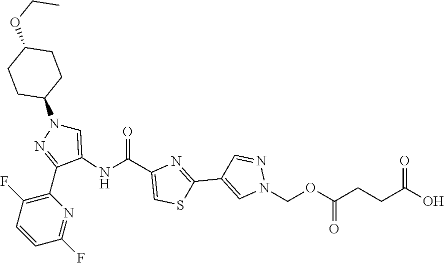 [SMILES: CCO[C@H]1CC[C@H](n2cc(NC(=O)c3csc(-c4cnn(COC(=O)CCC(=O)O)c4)n3)c(-c3nc(F)ccc3F)n2)CC1]

1H nmr (400 MHz, CDCl₃) δ 11.71 (1H, s, NH), 8.48 (1H, s, 1H of pyrazoleH-5, thiazoleH-5, pyrazoleH-3 or H-5), 8.29 (1H, s, 1H of pyrazoleH-5, thiazoleH-5, pyrazoleH-3 or H-5), 8.14 (1H, s, 1H of pyrazoleH-5, thiazoleH-5, pyrazoleH-3 or H-5), 8.06 (1H, s, 1H of pyrazoleH-5, thiazoleH-5, pyrazoleH-3 or H-5), 7.63 (1H, td, J 9.0, 6.5 Hz, pyridineH-4 or H-5), 6.88 (1H, ddd, J 8.5, 3.5, 2.5 Hz, pyridineH-4 or H-5), 6.11 (2H, s, OCH₂O), 4.26 (1H, tt, J 11.5, 4.0 Hz, cyclohexaneH-1 or H-4), 3.56 (2H, q, J 7.0 Hz, OCH₂CH₃), 3.37 (1H, tt, J 10.5, 4.0 Hz, cyclohexaneH-1 or H-4), 2.69 (4H, br s, COCH₂CH₂CO), 2.32-2.2.18 (4H, m, 4H of cyclohexaneH-2, H-3, H-5, H-6), 1.94-1.83 (2H, m, 2H of cyclohexaneH-2, H-3, H-5, H-6), 1.52-1.42 (2H, m, 2H of cyclohexaneH-2, H-3, H-5, H-6), 1.22 (3H, t, J 7.0 Hz, OCH₂CH₃); ¹³C nmr (100 MHz, CDCl₃) δ 175.8, 171.6, 159.8, 158.2, 157.5 (d, J 237.5 Hz), 153.4 (dd, J 260.5, 4.5 Hz), 150.1, 139.7 (d, J 5.0 Hz), 138.7 (t, J 14.5 Hz), 133.0 (d, J 8.5 Hz), 130.4 (d, J 5.0 Hz), 129.9 (dd, J 22.5, 9.0 Hz), 122.0, 121.8, 119.4, 118.6, 107.6 (dd, J 40.5, 5.5 Hz), 76.4, 72.4, 63.7, 61.5, 31.0, 30.9, 28.7, 28.5, 15.7; ¹⁹F nmr (380 MHz, CDCl₃) δ −72.5 dd, J 27.5, 9.5 Hz), −124.4 (ddd, J 28.5, 9.5, 2.5 Hz); m/z: 630 [M+H]⁺ (found [M+H]⁺, 630.1927, C₂₈H₂₉F₂N₇O₆S requires [M+H]⁺630.1941).

### VII-28: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl 2-morpholinoacetate

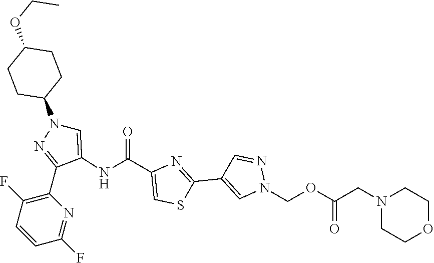 [SMILES: CCO[C@H]1CC[C@H](n2cc(NC(=O)c3csc(-c4cnn(COC(=O)CN5CCOCC5)c4)n3)c(-c3nc(F)ccc3F)n2)CC1]

1H nmr (400 MHz, CDCl₃) δ 8.50 (1H, s, 1H of pyrazoleH-5, thiazoleH-5, pyrazoleH-3 or H-5), 8.31 (1H, s, 1H of pyrazoleH-5, thiazoleH-5, pyrazoleH-3 or H-5), 8.17 (1H, s, 1H of pyrazoleH-5, thiazoleH-5, pyrazoleH-3 or H-5), 8.06 (1H, s, 1H of pyrazoleH-5, thiazoleH-5, pyrazoleH-3 or H-5), 7.65 (1H, td, J 9.0, 6.0 Hz, pyridineH-4 or H-5), 6.89 (1H, ddd, J 8.5, 3.0, 2.5 Hz, pyridineH-4 or H-5), 6.13 (2H, s, NCH₂O), 4.27 (1H, tt, J 11.5, 3.5 Hz, cyclohexaneH-1 or H-4), 3.73, 3.72 (4H, 2d AB system, J 4.5 Hz, 4H of morpholine), 3.56 (2H, q, J 7.0 Hz, OCH₂CH₃), 3.37 (1H, tt, J 10.5, 4.0 Hz, cyclohexaneH-1 or H-4), 3.29 (2H, s, COCH₂N), 2.57, 2.56 (4H, 2d AB system, J Hz, 4H of morpholine), 2.32-2.26 (2H, m, 2H of cyclohexaneH-2, H-3, H-5, H-6), 2.26-2.18 (2H, m, 2H of cyclohexaneH-2, H-3, H-5, H-6), 1.94-1.84 (2H, m, 2H of cyclohexaneH-2, H-3, H-5, H-6), 1.52-1.42 (2H, m, 2H of cyclohexaneH-2, H-3, H-5, H-6), 1.22 (3H, t, J 7.0 Hz, OCH₂CH₃); ¹⁹F nmr (380 MHz, CDCl₃) δ −72.6 (ddd, J 27.0, 7.0, 2.5 Hz), −124.4 ((ddd, J 27.0, 9.5, 2.5 Hz); m/z: 657 [M+H]⁺ (found [M+H]⁺, 657.2432, C₃₀H₃₄F₂N₈O₅S requires [M+H]⁺ 657.2414).

### VII-29: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl L-valinate

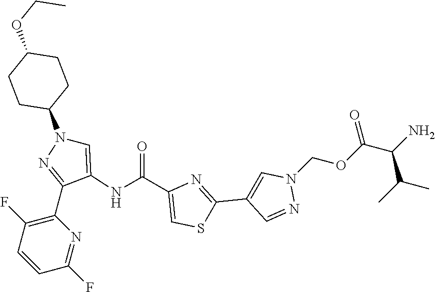 [SMILES: CCO[C@H]1CC[C@H](n2cc(NC(=O)c3csc(-c4cnn(COC(=O)[C@@H](N)C(C)C)c4)n3)c(-c3nc(F)ccc3F)n2)CC1]

¹H nmr (400 MHz, CDCl₃) δ 11.72 (1H, s, NH), 8.49 (1H, s, 1H of pyrazoleH-5, thiazoleH-5, pyrazoleH-3 or H-5), 8.31 (1H, s, 1H of pyrazoleH-5, thiazoleH-5, pyrazoleH-3 or H-5), 8.16 (1H, s, 1H of pyrazoleH-5, thiazoleH-5, pyrazoleH-3 or H-5), 8.05 (1H, s, 1H of pyrazoleH-5, thiazoleH-5, pyrazoleH-3 or H-5), 7.65 (1H, td, J 9.0, 6.5 Hz, pyridineH-4 or H-5), 6.88 (1H, dt, J 8.5, 3.0 Hz, pyridineH-4 or H-5), 6.14, 6.10 (2H, 2d AB system, J 10.5 Hz, NCH₂O), 4.26 (1H, tt, J 11.5, 4.0 Hz, cyclohexaneH-1 or H-4), 3.45 (2H, q, J 7.0 Hz, OCH₂CH₃), 3.40-3.32 (2H, m, cyclohexaneH-1 or H-4, COCHNH₂), 2.33-2.25 (2H, m, 2H of cyclohexaneH-2, H-3, H-5, H-6), 2.23-2.17 (2H, m, 2H of cyclohexaneH-2, H-3, H-5, H-6), 2.05-2.01 (1H, m, CHCH(CH₃)2), 1.94-1.83 (2H, m, 2H of cyclohexaneH-2, H-3, H-5, H-6), 1.51-1.41 (2H, m, 2H of cyclohexaneH-2, H-3, H-5, H-6), 1.22 (3H, t, J 7.0 Hz, OCH₂CH₃), 0.91 (3H, d, J 7.0 Hz, 1× CH₃ of CH(CH₃)₂), 0.82 (3H, d, J 6.5 Hz, 1× CH₃ of CH(CH₃)₂); ¹⁹F nmr (380 MHz, CDCl₃) δ −72.7, −124.4; m/z: 629 [M+H]⁺ (found [M+H]⁺, 629.2474, C₂₉H₃₄F₂N₈O₄S requires [M+H]⁺629.2465).

### VII-30: (4-(44(3-(3,6-difluoropyridin-2-yl)-1-((1r,4r)-4-ethoxycyclohexyl)-1H-pyrazol-4-yl)carbamoyl)thiazol-2-₃71)-1H-pyrazol-1-yl)methyl L-valinate benzenesulfonic acid

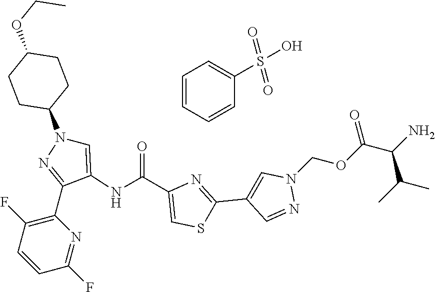 [SMILES: CCO[C@H]1CC[C@H](n2cc(NC(=O)c3csc(-c4cnn(COC(=O)[C@@H](N)C(C)C)c4)n3)c(-c3nc(F)ccc3F)n2)CC1 ; O=S(=O)(O)c1ccccc1]

1H nmr (400 MHz, D6-DMSO) δ 11.47 (1H, s, NH), 8.68 (1H, s, 1H of pyrazoleH-5, thiazoleH-5, pyrazoleH-3 or H-5), 8.53 (1H, s, 1H of pyrazoleH-5, thiazoleH-5, pyrazoleH-3 or H-5), 8.37 (1H, s, 1H of pyrazoleH-5, thiazoleH-5, pyrazoleH-3 or H-5), 8.27 (2H, br s, NH₂), 8.24 (1H, s, 1H of pyrazoleH-5, thiazoleH-5, pyrazoleH-3 or H-5), 8.09 (1H, td, J 9.5, 6.5 Hz, pyridineH-4 or H-5), 7.69-7.56 (2H, m, 2H of C₆H₅SO₃H), 7.32-7.24 (4H, m, 3H of C₆H₅SO₃H, pyridineH-4 or H-5), 6.34, 6.25 (2H, 2d AB system, J 11.0 Hz, NCH₂O), 4.33 (1H, tt, J 11.5, 3.5 Hz, cyclohexaneH-1 or H-4), 4.03 (1H, d, J 4.5 Hz, COCHNH₂), 3.47 (2H, q, J 7.0 Hz, OCH₂CH₃), 3.34 (1H, tt, J 10.5, 4.0 Hz, cyclohexaneH-1 or H-4), 2.14-2.06 (5H, m, CHCH(CH₃)₂, 4H of cyclohexaneH-2, H-3, H-5, H-6), 1.90-1.80 (2H, m, 2H of cyclohexaneH-2, H-3, H-5, H-6), 1.41-1.30 (2H, m, 2H of cyclohexaneH-2, H-3, H-5, H-6), 1.10 (3H, t, J 7.0 Hz, OCH₂CH₃), 0.89 (3H, d, J 6.5 Hz, 1× CH₃ of CH(CH₃)₂), 0.86 (3H, d, J 7.0 Hz, 1× CH₃ of CH(CH₃)₂); ¹⁹F nmr (380 MHz, D6-DMSO) δ −72.6, −124.5; m/z: 629 [M+H]⁺.

### VII-31: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl L-valinate methanesulfonic acid salt

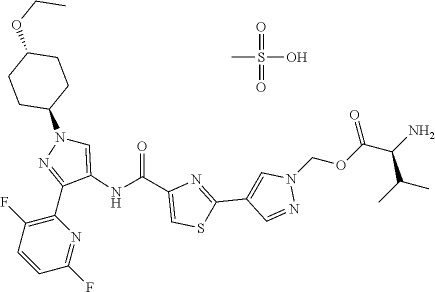

¹H nmr (400 MHz, D6-DMSO) δ 8.68 (1H, s, 1H of pyrazoleH-5, thiazoleH-5, pyrazoleH-3 or H-5), 8.53 (1H, s, 1H of pyrazoleH-5, thiazoleH-5, pyrazoleH-3 or H-5), 8.37 (1H, s, 1H of pyrazoleH-5, thiazoleH-5, pyrazoleH-3 or H-5), 8.34 (2H, br s, NH₂), 8.24 (1H, s, 1H of pyrazoleH-5, thiazoleH-5, pyrazoleH-3 or H-5), 8.09 (1H, dt, J 9.0, 6.5 Hz, pyridineH-4 or H-5), 7.26 (1H, ddd, J 9.0, 3.0, 2.5 Hz, pyridineH-4 or H-5), 6.34, 6.25 (2H, 2d AB system, J 11.0 Hz, NCH₂O), 4.33 (1H, tt, J 11.5, 3.0 Hz, cyclohexaneH-1 or H-4), 4.04 (1H, t, J 5.0 Hz, COCHNH₂), 3.47 (2H, q, J 7.0 Hz, OCH₂CH₃), 3.38-3.30 (1H, m, cyclohexaneH-1 or H-4), 2.31 (3H, s, CH₃SO₃H), 2.16-2.04 (5H, m, 4H of cyclohexaneH-2, H-3, H-5, H-6, CHCH(CH₃)₂), 1.91-1.80 (2H, m, 2H of cyclohexaneH-2, H-3, H-5, H-6), 1.40-1.30 (2H, m, 2H of cyclohexaneH-2, H-3, H-5, H-6), 1.10 (3H, t, J 7.0 Hz, OCH₂CH₃), 0.90 (3H, d, J 7.0 Hz, 1× CH₃ of CH(CH₃)₂), 0.86 (3H, d, J 7.0 Hz, 1× CH₃ of CH(CH₃)₂); ¹⁹F nmr (380 MHz, D6-DMSO) δ −73.0, −124.1; m/z: 629 [M+H]⁺.

### VII-35: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl (S)-2-amino-3,3-dimethylbutanoate

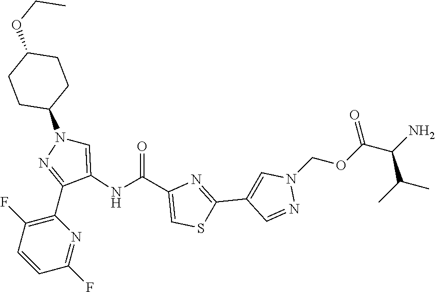 [SMILES: CCO[C@H]1CC[C@H](n2cc(NC(=O)c3csc(-c4cnn(COC(=O)[C@@H](N)C(C)C)c4)n3)c(-c3nc(F)ccc3F)n2)CC1]

¹H nmr (400 MHz, CDCl₃) δ 11.70 (1H, s, NH), 8.48 (1H, s, 1H of pyrazoleH-5, thiazoleH-5, pyrazoleH-3, H-5), 8.29 (1H, s, 1H of pyrazoleH-5, thiazoleH-5, pyrazoleH-3, H-5), 8.15 (1H, s, 1H of pyrazoleH-5, thiazoleH-5, pyrazoleH-3, H-5), 8.04 (1H, s, 1H of pyrazoleH-5, thiazoleH-5, pyrazoleH-3, H-5), 7.63 (1H, td, J 9.0, 6.5 Hz, pyridineH-4 or H-5), 6.86 (1H, ddd, J 9.0, 3.0, 2.5 Hz, pyridineH-4 or H-5), 6.13, 6.08 (2H, 2d AB system, J 10.5 Hz, NCH₂CO), 4.25 (1H, tt, J 11.5, 4.0 Hz, cyclohexaneH-1 or H-4), 3.54 (2H, q, J 7.0 Hz, OCH₂CH₃), 3.35 (1H, tt, J 11.0, 4.0 Hz, cyclohexaneH-1 or H-4), 3.20 (1H, s, COCH(C(CH₃)₃)NH₂), 2.32-2.24 (2H, m, 2H of cyclohexaneH-2, H-3, H-5, H-6), 2.24-2.16 (2H, m, 2H of cyclohexaneH-2, H-3, H-5, H-6), 1.93-1.82 (2H, m, 2H of cyclohexaneH-2, H-3, H-5, H-6), 1.50-1.40 (2H, m, 2H of cyclohexaneH-2, H-3, H-5, H-6), 1.20 (3H, t, J 7.0 Hz, OCH₂CH₃), 0.89 (9H, s, C(CH₃)3); ¹⁹F nmr (380 MHz, CDCl₃) δ −72.6, −124.4; m/z: 643 [M+H]⁺ (found [M+H]⁺, 643.2595, C₃₀H₃₇F₂N₈O₄S requires [M+H]⁺643.2621).

### VII-36: (4-(4-((3-(3,6-difluoropyridin-2-yl)-1-((1r,4r)-4-ethoxycyclohexyl)-1H-pyrazol-4-yl)carbamoyl)thiazol-2-₃4)-1H-pyrazol-1-yl)methyl (S)-2-amino-3,3-dimethylbutanoate benzenesulfonic acid

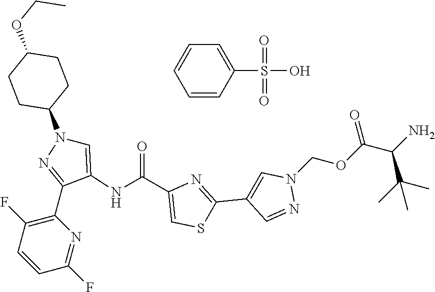 [SMILES: CCO[C@H]1CC[C@H](n2cc(NC(=O)c3csc(-c4cnn(COC(=O)[C@@H](N)C(C)(C)C)c4)n3)c(-c3nc(F)ccc3F)n2)CC1 ; O=S(=O)(O)c1ccccc1]

1H nmr (400 MHz, D₆-DMSO) δ 11.74 (1H, s, NH), 8.68 (1H, s, 1H of pyrazoleH-5, thiazoleH-5, pyrazoleH-3, H-5), 8.53 (1H, s, 1H of pyrazoleH-5, thiazoleH-5, pyrazoleH-3, H-5), 8.37 (1H, s, 1H of pyrazoleH-5, thiazoleH-5, pyrazoleH-3, H-5), 8.29 (2H, m, 2× NH₂), 8.25 (1H, s, 1H of pyrazoleH-5, thiazoleH-5, pyrazoleH-3, H-5), 8.09 (1H, dt, J 9.5, 6.5 Hz, pyridineH-4 or H-5), 7.59-7.56 (2H, m, 2H of C₆H₅SO₃H), 7.32-7.23 (4H, m, 3H of C₆H₅SO₃H, pyridineH-4 or H-5), 6.34, 6.26 (2H, 2d AB system, J 11.0 Hz, NCH₂CO), 4.33 (tt, J 11.5, 3.5 Hz, cyclohexaneH-1 or H-4), 3.91 (1H, br s, COCH(C(CH₃)₃)NH₂), 3.47 (2H, q, J 7.0 Hz, OCH₂CH₃), 3.34 (1H, tt, J 10.5, 3.5 Hz, cyclohexaneH-1 or H-4), 2.12-2.05 (4H, m, 4H of cyclohexaneH-2, H-3, H-5, H-6), 1.92-1.80 (2H, m, 2H of cyclohexaneH-2, H-3, H-5, H-6), 1.41-1.30 (2H, m, 2H of cyclohexaneH-2, H-3, H-5, H-6), 1.10 (3H, t, J 7.0 Hz, OCH₂CH₃), 0.93 (9H, s, C(CH₃)3); ¹³C nmr (100 MHz, D₆-DMSO) δ 168.5, 160.2, 157.5 (d, J 234.0 Hz), 157.5, 153.5 (d, J 258.0 Hz), 149.4, 148.9, 139.6 (d, J 7.5 Hz), 138.1 (d, J 14.5 Hz), 132.6 (d, J 9.0 Hz), 132.4 (d, J 3.0 Hz), 128.7, 128.0, 125.9, 124.4, 121.4, 120.3, 117.9, 76.0, 73.7, 63.0, 60.8, 33.7, 30.9 (2C), 26.4, 16.1; ¹⁹F nmr (380 MHz, D₆-DMSO) δ −72.9, −124.1; m/z: 643 [M+H]⁺.

### VII-37: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1- yl)methyl 4-(morpholinomethyl)benzoate

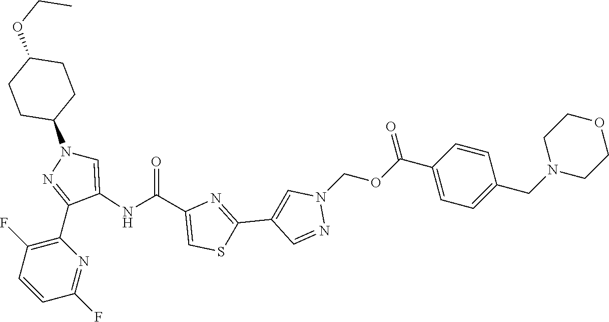 [SMILES: CCO[C@H]1CC[C@H](n2cc(NC(=O)c3csc(-c4cnn(COC(=O)c5ccc(CN6CCOCC6)cc5)c4)n3)c(-c3nc(F)ccc3F)n2)CC1]

¹H nmr (400 MHz, CDCl₃) δ 11.73 (1H, s, NH), 8.50 (1H, s, 1H of pyrazoleH-5, thiazoleH-5, pyrazoleH-3, H-5), 8.42 (1H, s, 1H of pyrazoleH-5, thiazoleH-5, pyrazoleH-3, H-5), 8.18 (1H, s, 1H of pyrazoleH-5, thiazoleH-5, pyrazoleH-3, H-5), 8.06 (1H, 1H of pyrazoleH-5, thiazoleH-5, pyrazoleH-3, H-5), 8.02 (2H, d, J 8.0 Hz, 2H of C₆H₄), 7.64 (1H, dt, J 9.0, 6.5 Hz, pyridineH-4 or H-5), 7.42 (1H, d, J 8.0 Hz, 2H of C₆H₄), 6.85 (1H, m, pyridineH-4 or H-5), 6.34 (2H, s, NCH₂CO), 4.27 (1H, tdd, J 11.5, 4.0, 3.5 Hz, cyclohexaneH-1 or H-4), 3.70, 3.69 (4H, 2d AB system, J 4.5 Hz, 4H of morpholine), 3.56 (2H, q, J 7.0 Hz, OCH₂CH₃), 3.54 (2H, s, C₆H₄CH₂N), 3.37 (1H, tt, J 10.5, 4.0 Hz, cyclohexaneH-1 or H-4), 2.42 (4H, br s, 4H of morpholine), 2.32-2.26 (2H, m, 2H of cyclohexaneH-2, H-3, H-5, H-6), 2.26-2.18 (2H, m, 2H of cyclohexaneH-2, H-3, H-5, H-6), 1.94-1.84 (2H, m, 2H of cyclohexaneH-2, H-3, H-5, H-6), 1.52-1.42 (2H, m, 2H of cyclohexaneH-2, H-3, H-5, H-6), 1.22 (3H, t, J 7.0 Hz, OCH₂CH₃); ¹⁹F nmr (380 MHz, CDCl₃) δ −72.5, −124.4; m/z: 733 [M+H]⁺.

### VII-39: (1R,2R)-2-(((4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)carbonyl)cyclohexane-1-carboxylic acid

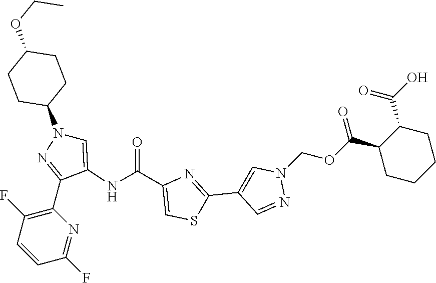 [SMILES: CCO[C@H]1CC[C@H](n2cc(NC(=O)c3csc(-c4cnn(COC(=O)[C@@H]5CCCC[C@H]5C(=O)O)c4)n3)c(-c3nc(F)ccc3F)n2)CC1]

¹H nmr (400 MHz, D6-DMSO) δ 12.25 (1H, br s, OH), 11.47 (1H, s, NH), 8.57 (1H, s, 1H of pyrazoleH-5, thiazoleH-5, pyrazoleH-3, H-5), 8.52 (1H, s, 1H of pyrazoleH-5, thiazoleH-5, pyrazoleH-3, H-5), 8.34 (1H, 1H of pyrazoleH-5, thiazoleH-5, pyrazoleH-3, H-5), 8.19 (1H, s, 1H of pyrazoleH-5, thiazoleH-5, pyrazoleH-3, H-5), 8.08 (1H, dt, J 9.0, 6.5 Hz, pyridineH-4 or H-5), 7.27 (1H, dt, J 8.5, 2.5 Hz, pyridineH-4 or H-5), 6.13, 6.05 (2H, 2d AB system, J 11.0 Hz, NCH₂O), 4.33 (1H, tt, J 11.5, 3.5 Hz, cyclohexaneH-1 or H-4), 3.47 (2H, q, J 7.0 Hz, OCH₂CH₃), 3.35 (1H, tt, J 11.0, 3.5 Hz, cyclohexaneH-1 or H-4), 2.78-2.40 (1H, m, cyclohexane dicarboxylic acid H-1 or H-2), 2.12-2.04 (4H, m, 4H of cyclohexaneH-2, H-3, H-5, H-6), 1.97-1.82 (1H, m, 1H of cyclohexane dicarboxylic acid H-1 or H-2), 1.90-1.81 (4H, m, 2H of cyclohexaneH-2, H-3, H-5, H-6, 2H of cyclohexane dicarboxylic acid H-3, H-4, H-5, H-6), 1.65 (2H, br s, cyclohexane dicarboxylic acid H-3, H-4, H-5, H-6), 1.39-1.30 (2H, m, 2H of cyclohexaneH-2, H-3, H-5, H-6), 1.27-1.17 (4H, m, 4H of cyclohexane dicarboxylic acid H-3, H-4, H-5, H-6), 1.10 (3H, t, J 7.0 Hz, OCH₂CH₃); ¹⁹F nmr (380 MHz, D6-DMSO) δ −72.8, −124.2; m/z: 684 [M+H]⁺ (found [M+H]⁺, 684.2416, C₃₂H₃₅F₂N₇O₆S requires [M+H]⁺684.2410).

### VII-40: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl (S)-2-amino-3,3-dimethylbutanoate methanesulfonic acid salt

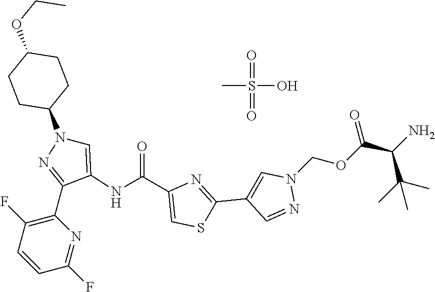

1H nmr (400 MHz, D6-DMSO) δ 12.47 (1H, br s, NH), 8.68 (1H, s, 1H of pyrazoleH-5, thiazoleH-5, pyrazoleH-3, H-5), 8.53 (1H, s, 1H of pyrazoleH-5, thiazoleH-5, pyrazoleH-3, H-5), 8.37 (1H, 1H of pyrazoleH-5, thiazoleH-5, pyrazoleH-3, H-5), 8.30 (2H, br s, NH₂), 8.25 (1H, s, 1H of pyrazoleH-5, thiazoleH-5, pyrazoleH-3, H-5), 8.09 (1H, dt, J 9.5, 6.5 Hz, pyridineH-4 or H-5), 7.27 (1H, dt, J 8.5, 2.5 Hz, pyridineH-4 or H-5), 6.34, 6.26 (2H, 2d AB system, J 11.0 Hz, NCH₂O), 4.33 (1H, tt, J 11.5, 3.5 Hz, 1H of cyclohexaneH-1 or H-4), 3.90 (1H, d, J 4.5 Hz, COCH(C(CH₃)3)NH₂), 3.47 (2H, q, J 7.0 Hz, OCH₂CH₃), 3.39-3.31 (1H, m, cyclohexaneH-1 or H-4), 2.30 (3H, s, CH₃SO₃H), 2.12-2.04 (4H, m, 4H of cyclohexaneH-2, H-3, H-5, H-6), 1.90-1.80 (2H, m, 2H of cyclohexaneH-2, H-3, H-5, H-6), 1.40-1.30 (2H, m, 2H of cyclohexaneH-2, H-3, H-5, H-6), 1.10 (3H, t, J 7.0 Hz, OCH₂CH₃), 0.93 (9H, s, C(CH₃)3); ¹³C nmr (100 MHz, D6-DMSO) δ 168.5, 160.2, 157.6, 157.5 (d, J 236.0 Hz), 155.7 (dd, J 260.0, 4.5 Hz), 149.4, 139.5 (d, J 6.5 Hz), 138.2 (t, J 14.0 Hz), 132.6 (d, J 8.5 Hz), 132.4, 124.4, 121.4, 120.3, 117.9, 76.0, 73.7, 65.4, 63.0, 60.8, 33.7, 30.9 (2C), 26.4, 16.1; ¹⁹F nmr (380 MHz, D6-DMSO) δ −72.9, −124.0; m/z: 643 [M+H]⁺.

### VII-42: N-(3-(3,6-difluoropyridin-2-yl)-1-((1r,4S)-4-ethoxycyclohexyl)-1H-pyrazol-4-yl)-2-(1-((2S,3S,4R,5R,6S)-3,4,5-trihydroxy-6-(hydroxymethyptetrahydro-2H-pyran-2-yl)-1H-pyrazol-4-yl)thiazole-4-carboxamide

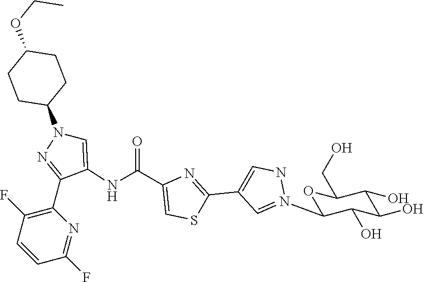

¹H nmr (400 MHz, D₆-DMSO) δ 11.47 (1H, s, NH), 8.66 (1H, s, 1H of pyrazoleH-5, thiazoleH-5, pyrazoleH-3, H-5), 8.53 (1H, s, 1H of pyrazoleH-5, thiazoleH-5, pyrazoleH-3, H-5), 8.32 (1H, s, 1H of pyrazoleH-5, thiazoleH-5, pyrazoleH-3, H-5), 8.14 (1H, s, 1H of pyrazoleH-5, thiazoleH-5, pyrazoleH-3, H-5), 8.08 (1H, td, J 9.5, 6.5 Hz, pyridineH-4 or H-5), 7.26 (1H, dt, J 8.5, 2.5 Hz, pyridineH-4 or H-5), 5.30 (1H, d, J 6.0 Hz, OH-2), 5.23-5.21 (2H, m, H-1, OH-3), 5.09 (1H, d, J 5.5 Hz, OH-4), 4.61 (1H, t, J 5.5 Hz, OH-6), 4.33 (1H, br t, J 11.5 Hz, cHexH-1 or H-4), 3.79 (1H, td, J 9.0, 6.0 Hz, H-2), 3.70 (1H, dd, J 11.0, 5.5 Hz, 1× H-6), 3.47 (2H, q, J 7.0 Hz, OCH₂CH₃), 3.45-3.32 (3H, m, cHexH-1 or H-4, H-3, 1× H-6), 3.24-3.21 (1H, m, H-4), 2.12-2.04 (4H, m, 4H of cHexH-2, H-3, H-5, H-6), 1.91-1.81 (1H, m, 2H of cHexH-2, H-3, H-5, H-6), 1.40-1.31 (2H, m, 2H of cHexH-2, H-3, H-5, H-6), 1.10 (3H, t, J 7.0 Hz, OCH₂CH₃); ¹⁹F nmr (380 MHz, D₆-DMSO) δ −72.8, −124.2; m/z: 662 [M+H]⁺ (found [M+H]⁺, 662.2219, C₂₉H₃₃F₂N₇O₇S requires [M+H]⁺ 662.2203).

### VII-43: N-(3-(3,6-difluoropyridin-2-yl)-1-((1r,4R)-4-ethoxycyclohexyl)-1H-pyrazol-4-yl)-2-(1-((2R,3R,4R,5R,6S)-3,4,5-trihydroxy-6-(hydroxymethyptetrahydro-2H-pyran-2-yl)-1H-pyrazol-4-yl)thiazole-4-carboxamide

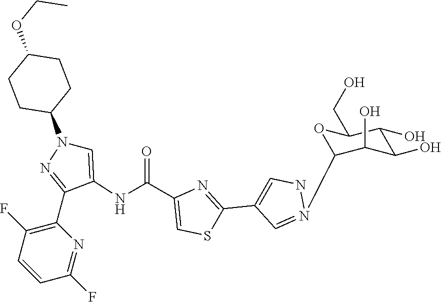

¹H nmr (400 MHz, D₆-DMSO) δ 11.49 (1H, s, NH), 8.59 (1H, s, 1H of pyrazoleH-5, thiazoleH-5, pyrazoleH-3, H-5), 8.53 (1H, s, 1H of pyrazoleH-5, thiazoleH-5, pyrazoleH-3, H-5), 8.33 (1H, s, 1H of pyrazoleH-5, thiazoleH-5, pyrazoleH-3, H-5), 8.17 (1H, s, 1H of pyrazoleH-5, thiazoleH-5, pyrazoleH-3, H-5), 8.09 (1H, td, J 9.5, 6.0 Hz, pyridineH-4 or H-5), 7.28 (1H, dt, J 8.5, 2.5 Hz, pyridineH-4 or H-5), 5.70 (1H, d, J 4.0 Hz, H-1), 5.15 (1H, br s, 1× OH), 4.93 (2H, br m, 2× OH), 4.54 (1H, br s, 1× OH), 4.39 (1H, t, J 3.5 Hz, H-2), 4.33 (1H, brt, J 11.5 Hz, cHexH-1 or H-4), 3.91 (1H, dd, J 7.0, 3.0 Hz, H-3), 3.63 (1H, d, J 10.0 Hz, 1× H-6), 3.58-3.52 (2H, m, H-4, 1× H-6), 3.47 (2H, q, J 7.0 Hz, OCH₂CH₃), 3.45-3.42 (1H, m, H-5), 3.35 (1H, m, cHexH-1 or H-4), 2.12-2.04 (4H, m, 4H of cHexH-2, H-3, H-5, H-6), 1.92-1.81 (2H, m, 2H of cHexH-2, H-3, H-5, H-6), 1.40-1.31 (2H, m, 2H of cHexH-2, H-3, H-5, H-6), 1.10 (3H, t, J 7.0 Hz, OCH₂CH₃); ¹⁹F nmr (380 MHz, D6-DMSO) δ −72.7, −124.2; m/z: 662 [M+H]⁺ (found [M+H]⁺, 662.2195, C₂₉H₃₃F₂N₇O₇S requires [M+H]⁺ 662.2203).

### VII-49: 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl hydrogen phosphate tris salt

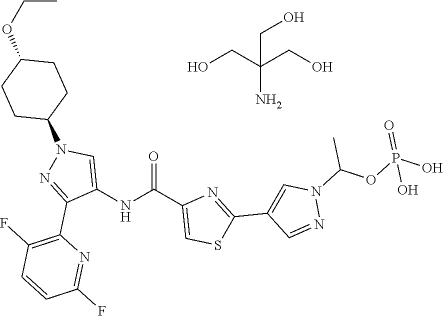 [SMILES: CCO[C@H]1CC[C@H](n2cc(NC(=O)c3csc(-c4cnn(C(C)OP(=O)(O)O)c4)n3)c(-c3nc(F)ccc3F)n2)CC1 ; NC(CO)(CO)CO]

1H nmr (400 MHz, D6-DMSO) δ 11.46 (1H, s, NH), 8.51 (1H, s, 1H of pyrazoleH-5, thiazoleH-5, pyrazoleH-3, H-5), 8.49 (1H, s, 1H of pyrazoleH-5, thiazoleH-5, pyrazoleH-3, H-5), 8.28 (1H, s, 1H of pyrazoleH-5, thiazoleH-5, pyrazoleH-3, H-5), 8.07 (1H, s, 1H of pyrazoleH-5, thiazoleH-5, pyrazoleH-3, H-5), 8.06 (1H, dt, J 10.0, 6.5 Hz, pyridineH-4 or H-5), 7.28 (1H, dt, J 8.5, 2.5 Hz, pyridineH-4 or H-5), 6.12 (1H, dq, J 9.0, 6.0 Hz, NCH(CH₃)OP), 4.32 (1H, brt, J 11.5 Hz, cyclohexaneH-1 or H-4), 3.47 (2H, q, J 7.0 Hz, OCH₂CH₃), 3.44 (6H, s, C(CH₂OH)₃), 3.35 (1H, tt, J 10.5, 3.5 Hz, cyclohexaneH-1 or H-4), 2.12-2.05 (4H, m, 4H of cyclohexaneH-2, H-3, H-5, H-6), 1.91-1.81 (2H, m, 2H of cyclohexaneH-2, H-3, H-5, H-6), 1.66 (3H, d, J 6.0 Hz, NCH(CH₃)0P), 1.40-1.30 (2H, m, 2H of cyclohexaneH-2, H-3, H-5, H-6), 1.10 (3H, t, J 7.0 Hz, OCH₂CH₃); ³²P nmr (380 MHz, D6-DMSO) δ 0.2; ¹⁹F nmr (380 MHz, D6-DMSO) δ −72.6, −124.4; m/z: 624 [M+H]⁺.

### VII-50: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl glycinate benzenesulfonic acid salt

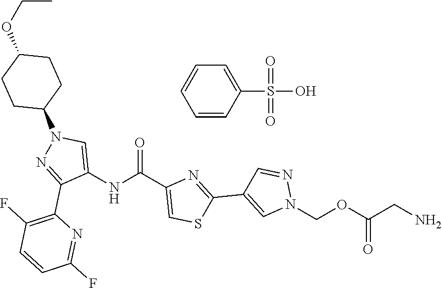 [SMILES: CCO[C@H]1CC[C@H](n2cc(NC(=O)c3csc(-c4cnn(COC(=O)CN)c4)n3)c(-c3nc(F)ccc3F)n2)CC1 ; O=S(=O)(O)c1ccccc1]

¹H nmr (400 MHz, D6-DMSO) δ 11.47 (1H, s, NH), 8.67 (1H, s, 1H of pyrazoleH-5, thiazoleH-5, pyrazoleH-3, H-5), 8.53 (1H, s, 1H of pyrazoleH-5, thiazoleH-5, pyrazoleH-3, H-5), 8.37 (1H, s, 1H of pyrazoleH-5, thiazoleH-5, pyrazoleH-3, H-5), 8.24 (1H, s, 1H of pyrazoleH-5, thiazoleH-5, pyrazoleH-3, H-5), 8.23 (2H, br s, NH₂), 8.09 (1H, dt, J 9.5, 6.5 Hz, pyridineH-4 or H-5), 7.59-7.56 (2H, m, 2H of C₆H₅SO₃H), 7.32-7.25 (4H, m, 3H of C₆H₅SO₃H, pyridineH-4 or H-5), 6.26 (2H, s, NCH₂CO), 4.34 (1H, tt, J 11.5, 3.5 Hz, cyclohexaneH-1 or H-4), 3.92 (2H, br s, COCH₂NH₂), 3.47 (2H, q, J 7.0 Hz, OCH₂CH₃), 3.39-3.33 (1H, m, cyclohexaneH-1 or H-4), 2.12-2.05 (4H, m, 4H of cyclohexaneH-2, H-3, H-5, H-6), 1.91-1.80 (2H, m, 2H of cyclohexaneH-2, H-3, H-5, H-6), 1.41-1.30 (2H, m, 2H of cyclohexaneH-2, H-3, H-5, H-6), 1.10 (3H, t, J 7.0 Hz, OCH₂CH₃); ¹⁹F nmr (380 MHz, D6-DMSO) δ −73.0, −124.1; m/z: 587 [M+H]⁺.

### VII-56: 4-((4(4-(4-(3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methoxy)-4-oxobutanoic acid tris salt

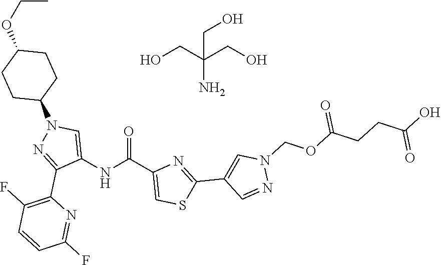 [SMILES: CCO[C@H]1CC[C@H](n2cc(NC(=O)c3csc(-c4cnn(COC(=O)CCC(=O)O)c4)n3)c(-c3nc(F)ccc3F)n2)CC1 ; NC(CO)(CO)CO]

¹ H nmr (400 MHz, D₂O) δ 7.52 (1H, s, 1H of pyrazoleH-5, thiazoleH-5, pyrazoleH-3, H-5), 7.49 (1H, s, 1H of pyrazoleH-5, thiazoleH-5, pyrazoleH-3, H-5), 7.16 (1H, s, 1H of pyrazoleH-5, thiazoleH-5, pyrazoleH-3, H-5), 7.13 (1H, s, 1H of pyrazoleH-5, thiazoleH-5, pyrazoleH-3, H-5), 7.13-7.07 (1H, m, pyridineH-4 or H-5), 6.24 (1H, br d, J 8.0 Hz, pyridineH-4 or H-5), 5.69 (2H, s, NCH₂O), 7.39 (1H, br t, J 11.5 Hz, cyclohexaneH-1 or H-4), 3.59 (6H, s, 3× CCH₂OH), 3.55 (2H, q, J 7.0 Hz, OCH₂CH₃), 3.37 (1H, br t, J 10.5 Hz, cyclohexaneH-1 or H-4), 2.54 (2H, t, J 6.5 Hz, 2H of COCH₂CH₂CO), 2.39 (2H, t, J 6.5 Hz, 2H of COCH₂CH₂CO), 2.12-2.04 (2H, m, 2H of cyclohexaneH-2, H-3, H-5, H-6), 2.15-1.98 (2H, m, 2H of cyclohexaneH-2, H-3, H-5, H-6), 1.55-1.44 (2H, m, 2H of cyclohexaneH-2, H-3, H-5, H-6), 1.32-1.21 (2H, m, 2H of cyclohexaneH-2, H-3, H-5, H-6), 1.10 (3H, t, J 7.0 Hz, OCH₂CH₃); ¹⁹F nmr (380 MHz, D₂O) 6-73.4, −124.7; m/z: 630 [M+H]⁺.

### VII-68: N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide citric acid cocrystal

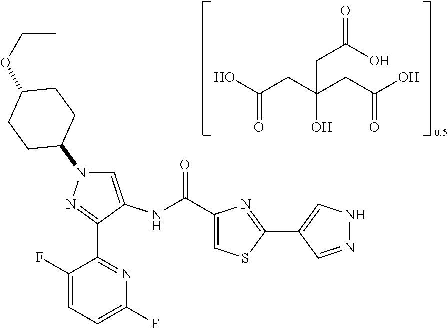

¹H nmr (400 MHz, D6-DMSO) δ 8.53 (1H, s, thiazoleH-5 or pyrazoleH-5), 8.29 (3H, s, pyrazoleH-3, H-5, thiazoleH-5 or pyrazoleH-5), 8.08 (1H, td, J 9.0, 6.0 Hz, pyridineH-4 or H-5), 7.29 (1H, ddd, J 9.0, 3.0, 2.5 Hz, pyridineH-4 or H-5), 5.14 (0.5H, br s, COH), 4.33 (1H, tt, J 11.5, 3.5 Hz, cyclohexaneH-1 or H-4), 3.47 (2H, q, J 7.0 Hz, OCH₂CH₃), 3.35 (1H, m, cyclohexaneH-1 or H-4), 2.74, 2.64 (3H, 2d AB system, J 15.5 Hz, 3×0.5 CCH₂CO₂H), 2.08 (4H, m, 4H of cyclohexaneH-2, H-3, H-5, H-6), 1.85 (2H, m, 2H of cyclohexaneH-2, H-3, H-5, H-6), 1.35 (2H, m, 2H of cyclohexaneH-2, H-3, H-5, H-6), 1.10 (3H, t, J 7.0 Hz, OCH₂CH₃); ¹⁹F nmr (380 MHz, D6-DMSO) δ −73.0, −124.2; m/z: 500 [M+H]⁺.

### VII-69: (4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)methyl dihydrogen phosphate bis(tris(hydroxymethyl)aminomethanel salt

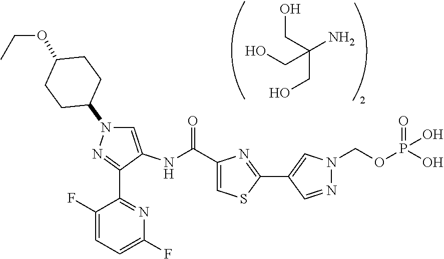

¹H nmr (400 MHz, D₂O) δ 7.89 (1H, s, thiazoleH-5 or pyrazoleH-5), 7.80 (1H, s, thiazoleH-5 or pyrazoleH-5), 7.45 (1H, s, pyrazoleH-3 or H-5), 7.44 (1H, s, pyrazoleH-3 or H-5), 7.33 (1H, m, pyridineH-4 or H-5), 6.53 (1H, d, J 9.0 Hz, pyridineH-4 or H-5), 5.51 (1H, d, J 6.5 Hz, NCH₂OP), 3.93 (1H, tt, J 12.0, 3.0 Hz, cyclohexaneH-1 or H-4), 3.58 (2H, q, J 7.0 Hz, OCH₂CH₃), 3.57 (12H, s, 2× H₂NC(CH₂OH)3), 3.45 (1H, m, cyclohexaneH-1 or H-4), 2.14 (2H, br d, J 10.5 Hz, 2H of cyclohexaneH-2, H-3, H-5, H-6), 2.03 (2H, br d, J 12.0 Hz, cyclohexaneH-2, H-3, H-5, H-6), 1.63 (2H, m, 2H of cyclohexaneH-2, H-3, H-5, H-6), 1.32 (2H, m, 2H of cyclohexaneH-2, H-3, H-5, H-6), 1.11 (3H, t, J 7.0 Hz, OCH₂CH₃); ³¹1³ nmr (162 MHz, D₂O) δ 1.05; ¹⁹F nmr (380 MHz, D₂O) 6-72.8 (d, 26.0 Hz), −124.7 (dd, J 27.0, 9.5 Hz); m/z: 610 [M+H]⁺ (found [M+H]⁺, 610.1432, C₂₄H₂₆F₂N₇O₆PS requires [M+H]⁺610.1444).

### VII-70: benzyl ((S)-1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)-4-methyl-1-oxopentan-2-yl)carbamate

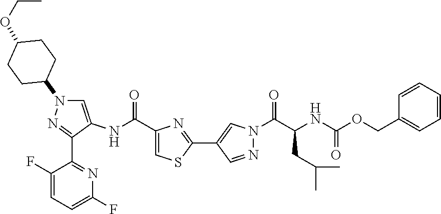

¹H nmr (400 MHz, CDCl₃) δ 8.78 (1H, s, 1H of pyrazoleH-3, H-5), 8.50 (1H, s, thiazoleH-5 or pyrazoleH-5), 8.35 (1H, s, 1H of pyrazoleH-3, H-5), 8.14 (1H, s, thiazoleH-5 or pyrazoleH-5), 7.65 (1H, td, J 9.0, 6.0 Hz, pyridineH-4 or H-5), 7.35-7.30 (5H, m, C₆H₅), 6.90 (1H, ddd, J 9.0, 3.0, 2.5 Hz, pyridineH-4 or H-5), 5.66 (1H, m, NCHCO), 5.50 (1H, d, J 9.0 Hz, NH), 5.14, 5.11 (2H, 2d AB system, J 12.5 Hz, OCH₂C₆H₅), 4.27 (1H, tt, J 11.5, 4.0 Hz, cycohexaneH-1 or H-4), 3.56 (2H, q, J 7.0 Hz, OCH₂CH₃), 3.37 (1H, tt, J 10.5, 4.0 Hz, cyclohexaneH-1 or H-4), 2.29 (2H, br d, J 12.0 Hz, 2H of cyclohexaneH-2, H-3, H-5, H-6), 2.22 (2H, m, 2H of cyclohexaneH-2, H-3, H-5, H-6), 1.89 (2H, m, 2H of cyclohexaneH-2, H-3, H-5, H-6), 1.82 (2H, m, CHCH₂CH(CH₃)₂), 1.65 (1H, m, CHCH₂CH(CH₃)₂), 1.47 (2H, m, 2H of cyclohexaneH-2, H-3, H-5, H-6), 1.22 (3H, t, J 7.0 Hz, OCH₂CH₃), 1.07 (2H, br d, J 5.5 Hz, 1× CH(CH₃)₂), 0.96 (3H, d, J 6.0 Hz, 1× CH(CH₃)₂); ¹⁹F nmr (380 MHz, CDCl₃) δ −72.5 (d, J 27.5 Hz), −124.4 (dd, J 27.0, 9.5 Hz); m/z: 769 [M+Na]⁺, 747 [M+H]⁺ (found [M+H]⁺, 747.2885, C₃₇H₄₀F₂N₈O₅S requires [M+H]⁺747.2883).

### Example 4

### Compound Screening Protocol using Dendritic Cells (DC)

A. Materials

Human PBMC cells (PPA Research Group, Cat No. 15-00021); RPMI media 10% FBS; GMCSF (Peprotech, Cat No. 300-03) and IL4 (Peprotech Cat No. 200-04); White clear bottom 96 well plates (Fisher, Cat No. 07-200-587, Corning #3903); Human IL-2 DuoSet ELISA (R&D Systems, Cat No. DY202); Human IL-6 DuoSet ELISA (R&D Systems, Cat No. DY206); Cell Titer Glo reagent (Promega, Cat No. G7573); Dynabeads Human T-Activator CD3/CD28 (Fisher, Cat No. 111.61D); Anti-human CD3 (BD Biosciences, Cat No. 555336); CD28, Clone CD28.2 (Beckman Coulter Inc. Cat No. IM1376); Recombinant Human IL-2 Protein (R&D Systems, Cat No. 202-IL-500).

B. Differentiation of Dendritic Cells

Human peripheral blood mononuclear cells (PBMC) (400 million) obtained from the vendor were transferred into three T-175 flasks containing 16 ml RPMI media (10% fetal bovine serum (FBS)) and incubated for 2 hours at 37° C. After 2 hours, floating PBL was removed and the cell was rinsed twice with 10 ml of media. The PBL and media was saved for T cell expansion. 16 ml of fresh RPMI media (10% FBS) containing Granulocyte Macrophage Colony—Stimulating Factor (GMCSF) (100 ng/ml) and IL4 (20 ng/ml) was added and the flask was kept in a 37° C. incubator. After 3 days, fresh GMCSF (100 ng/ml) and IL4 (20 ng/ml) was added to the flask and the incubation was continued.

C. Expansion of T cells

T-175 flask was coated with 16 mls of PBS with 1 μg/m1 anti-CD3 (16 μl of 1 mg/ml stock) and 5 μg/m1 anti-CD28 (400 μl of 200 μg/m1 stock) for about 2 hours. After spinning down, 2×10⁸ PBL was resuspended into 60 mls of RPMI media (10% FBS) with 60 μl IL2. The coating solution was aspirated off from flask and cells were added to the stimulation flask. After 3 days, the stimulation flask was knocked to dislodge any cells stuck on the bottom of the flask. And a new T-175 flask was reseeded in 60 mls media with 60 μl IL2 at 1×10⁶ cells/ml.

D. CRS Assay

After 4 days, the dendritic cells were harvested by spinning down (1000 rpm/10 min) and aspirating the media. After resuspending the cells in fresh RPMI media (10% FBS), the cells were plated (25K/well in 50 μl) onto a white clear bottom 96 well plate. 100 μl of RPMI media containing 2× concentrated test compound was added per well to the above cell-culture media (final concentration becomes 1×) and the plates were pre-incubated for 1 hour at 37° C.

After 1 hour compound pre-incubation, 50 μl per well of T cells (1.7k/well) was added with CD3/CD28 beads (1.7k/well), and the plates were incubated at 37° C. overnight.

After incubation, 80 μl of the supernatant was harvested from each well for IL6 ELISA and 80 μl of the supernatant for IL2 ELISA. ELISAs were carried out per instructions from R&D Systems. To the remaining 40 μl/well of the cell culture plate 25 μl of Cell Titer Glo reagent was added, and the mixture was incubated for 1-2 minutes on a shaker. The plate was read for luminescence intensity to determine the compound cytotoxicity. The results are shown in Table 1.

TABLE 1

| | | Dendritic | Dendritic | |
|---|---|---|---|---|
| | | cells + T | cells + T | |
| | | cells + | cells + | |
| | | CD3/CD28 | CD3/CD28 | |
| | | beads | beads | Cell Titer |
| | | IL6* ELISA | IL2* ELISA | Glo 2 |
| Compound | Target | EC₅₀ (μM) | EC₅₀ (μM) | EC₅₀ (μM) |
| I-432 | JAK | 0.052 | 4.41 | 4.55 |
| VI-176 | IRAK1/4 | 0.195 | 6.9 | 11.95 |
| Tofacitinib | JAK | 0.108 | ND** | ND** |
| Acalabrutinib | Btk | ND** | ND** | ND** |

*IL-6 is primarily produced by the dendritic cells activated by the T cells, and IL-2 is only produced by the activated T cells.
**ND indicates that an accurate inhibition curve may not have been produced due to compound insolubility, artifacts in the assay, and/or other factors.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method for treating and/or preventing cytokine release syndrome (CRS), the method comprising administering to a subject experiencing, or at risk of developing, CRS an effective amount of a compound having activity as a kinase inhibitor.

2. The method of claim 1, wherein the compound is a JAK inhibitor, an IRAK inhibitor, or a combination thereof

3. The method of claim 1, wherein the compound is a pyrimidine diamine compound according to Formula I 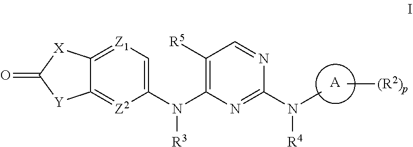
or a salt, solvate, or N-oxide thereof, wherein:
X and Y are each independently O, S, S(O), SO₂ or NR¹;
each R¹ is independently for each occurrence H, C₁₋₆alkyl, C(O)—C₁₋₆alkyl, CO₂—C₁₋₆alkyl or R⁵⁰;
each R⁵⁰ is C(R⁹)₂—O—R¹⁰ or C(R⁹)₂—S—R¹⁰;
each R⁹ is independently for each occurrence H, C₁₋₆alkyl, C₆₋₁₀aryl or C₇₋₁₆arylalkyl; or alternatively, two R⁹, together with the carbon to which they are attached, form a C₃₋₈cycloalkyl group or a 3-8 membered heterocycloaliphatic; R¹⁰ is R^{a} or —P(O)(OR¹¹)₂; each R¹¹ is independently for each occurrence R^{a} or a monovalent cationic group; or two R¹¹, together with the atoms to which they are attached, form a 4-8 membered cyclic phosphate group, or two R¹¹ together represent a divalent cationic group;
ring A is a C₆₋₁₀aryl or a 5-10 membered heteroaryl;
each R² is independently for each occurrence H, R^{e}, R^{b}, R^{e} substituted with one or more of the same or different R^{a} and/or R^{b}, —OR^{e} substituted with one or more of the same or different R^{a} and/or R^{b}, —SR^{e} substituted with one or more of the same or different R^{a} and/or R^{b}, —C(O)R^{e} substituted with one or more of the same or different R^{a} and/or R^{b}, —N(R^{a})R^{e} where R^{a} is substituted with one or more of the same or different R^{a} and/or R^{b}, —S(O)₂R^{e} substituted with one or more of the same or different R^{a} and/or R^{b}, —B(OR^{a})₂, —B(N(R^{c})₂)₂, —(C(R^{a})₂)ₘ—R^{b}, —O—(C(R^{a})₂)ₘ—R^{b}, —S—(C(R^{a})₂)ₘ—R^{b}, —O—(C(R^{b})₂)ₘ—R^{a}, —N(R^{a})—(C(R^{a})₂)ₘ—R^{b}, —O—(CH₂)ₘ—CH((CH₂)ₘ—R^{b})R^{b}, —C(O)N(R^{a})—(C(R^{a})₂)ₘ—R^{b}, —O—(C(R^{a})₂)ₘ—C(O)N(R^{a})—(C(R^{a})₂)ₘ—R^{b}, —N((C(R^{a})₂)ₘR^{b})₂, —S—(C(R^{a})₂)ₘ—C(O)N(R^{a})—(C(R^{a})₂)ₘ—R^{b}, —N(R^{a})—C(O)—N(R^{a})—(C(R^{a})₂)ₘ—R^{b}, —N(R^{a})—C(O)—(C(R^{a})₂)ₘ—C(R^{a})(R^{b})₂ or —N(R^{a})—(C(R^{a})₂)ₘ—C(O)—N(R^{a})—(C(R^{a})₂)ₘ—R^{b};
each R^{a} is independently for each occurrence H, deuterium, C₁₋₆alkyl, C₃₋₈cycloalkyl, C₄-ncycloalkylalkyl, C₆₋₁₀aryl, C₇₋₁₆arylalkyl, 2-6 membered heteroalkyl, 3-10 membered heterocycloaliphatic, 4-11 membered heterocycloaliphaticalkyl, 5-15 membered heteroaryl or 6-16 membered heteroarylalkyl;
each R^{b} is independently for each occurrence ═O, —OR^{a}, —O—(C(R^{a})₂)ₘ—OR^{a}, haloC₁₋₃alkyloxy, ═S, —SR^{a}, ═NR^{a}, ═NOR^{a}, —N(R^{c})₂, halo, —CF₃, —CN, —NC, —OCN, —SCN, —NO, —NO₂, ═N₂, —N₃, —S(O)R^{a}, —S(O)₂R^{a}, —SO₃R^{a}, —S(O)N(R^{c})₂, —OS(O)R^{a}, —OS(O)₂R^{a}, —OSO₃R^{a}, —OS(O)₂N(R^{c})₂, —C(O)R^{a}, —CO₂R^{a}, —C(O)N(R^{c})₂, —C(NR^{a})—N(R^{c})₂, —C(NOH)—R^{a}, —C(NOH)—N(R^{a})₂, —OC(O)R^{a}, —OC(O)OR^{a}, —OC(O)N(R^{c})₂, —OC(NH)—N(R^{c})₂, —OC(NR^{a})—N(R^{c})₂, —[N(R^{a})C(O)]ₙR^{a}, —[N(R^{a})C(O)]ₙOR^{a}, —[N(R^{a})C(O)]ₙN(R^{c})₂ or —[N(R^{a})C(NR^{a})]ₙ—N(R^{c})₂;
each R^{c} is independently for each occurrence R^{a}, or, alternatively, two R^{c} are taken together with the nitrogen atom to which they are bonded to form a 3 to 10-membered heterocycloaliphatic or a 5-10 membered heteroaryl which may optionally include one or more of the same or different additional heteroatoms and which is optionally substituted with one or more of the same or different R^{a} and/or R^{d} groups;
each R^{d} is ═O, —OR^{a}, haloC₁₋₃alkyloxy,C₁₋₆alkyl,═S, —SR^{a}, ═NR^{a}, ═NOR^{a}, —N(R^{a})₂, halo, —CF₃, —CN, —NC, —OCN, —SCN, —NO, —NO₂, ═N₂, —N₃, —S(O)R^{a}, —S(O₂)R^{a}, —SO₃R^{a}, —S(O)N(R^{a})₂, —S(O)₂N(R^{a})₂, —OS(O)R^{a}, —OS(O)₂R^{a}, —OSO₃R^{a}, —OS(O)₂N(R^{a})₂, —C(O)R^{a}, —CO₂R^{a}, —C(O)N(R^{a})₂, —C(NR^{a})N(R^{a})₂, —C(NOH)R^{a}, —C(NOH)N(R^{a})₂, —OCO₂R^{a}, —OC(O)N(R^{a})₂, —OC(NR^{a})N(R^{a})₂, —[N(R^{a})C(O)]ₙR^{a}, —(C(R^{a})₂)ₙ—OR^{a}, —N(R^{a}) —S(O)₂R^{a}, —C(O)—C₁₋₆ haloalkyl, —S(O)₂C₁₋₆ haloalkyl, —OC(O)R^{a}, —O(C(R^{a})₂)ₘ—OR^{a}, —S(C(R^{a})₂)ₘ—OR^{a}, —N(R^{a})C₁₋₆haloalkyl, —P(O)(OR^{a})₂, —N(R^{a})—(C(R^{a})₂)ₘ—OR^{a}, —[N(R^{a})C(O)]ₙOR^{a}, —[N(R^{a})C(O)]ₙN(R^{a})₂, —[N(R^{a})C(NR^{a})]ₙN(R^{a})₂ or —N(R^{a})C(O)C₁₋₆ haloalkyl; or two R^{d}, taken together with the atom or atoms to which they are attached, combine to form a 3-10 membered partially or fully saturated mono or bicyclic ring, optionally containing one or more heteroatoms and optionally substituted with one or more R^{a};
each R^{e} is independently for each occurrence C₁₋₆alkyl, C₃₋₈cycloalkyl, C₄₋₁₁ cycloalkylalkyl, C₆₋₁₀aryl, C₇₋₁₆arylalkyl, 2-6 membered heteroalkyl, 3-10 membered heterocycloaliphatic, 4-11 membered heterocycloaliphaticalkyl, 5-15 membered heteroaryl or 6-16 membered heteroarylalkyl;
p is 0, 1, 2,3 or 4;
each m is 1, 2 or 3;
each n is 0, 1, 2 or 3;
or two R² groups, taken together with the atom or atoms to which they are attached, combine to form a 4-10 membered partially or fully saturated mono or bicyclic ring, optionally containing one or more heteroatoms and optionally substituted with one or more R^{a} and/or R^{b};
Z¹ and Z² are each independently CH, CR² or N;
R³ is H, C₁₋₆alkyl or R⁵⁰;
R⁴ is H, C₁₋₆alkyl or R⁵⁰; and
R⁵ is halo, —CN, C₁₋₆alkyl, alkynyl, hydroxy, C₁₋₆alkoxy, nitro, —N(R^{a})₂, —C(O)N(R^{a})₂, —CO₂R^{a} or —C(O)R^{a}.

4. The method of claim 3, wherein the compound has a formula according to: 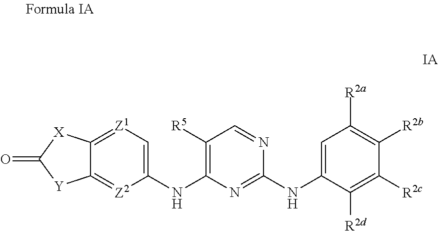
or a salt, solvant, or N-oxide thereof, wherein each of R^{2a}, R^{2b}, R^{2c} and R^{2d} is independently for each occurrence as previous defined for R²; 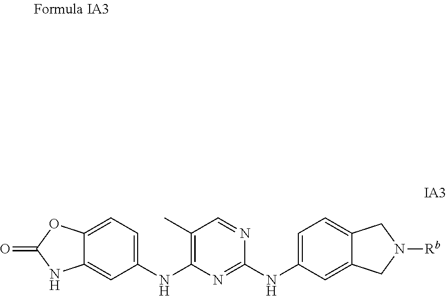
or a salt, solvate, N-oxide or prodrug thereof, wherein R^{b} is OH, C₁₋₆alkyl, —CO₂C₁₋₆alkyl, —C(O)C₁₋₆alkyl or —S(O)₂C₁₋₆alkyl; 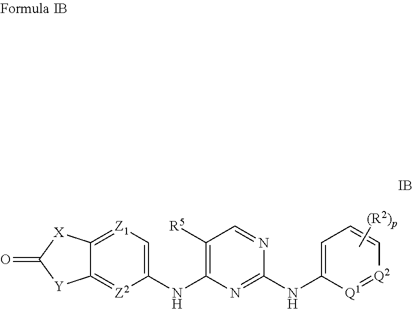
or a salt, solvate, N-oxide or prodrug thereof, wherein Q¹ and Q² are each independently N or CH provided at least one of Q¹ and Q² is N; or 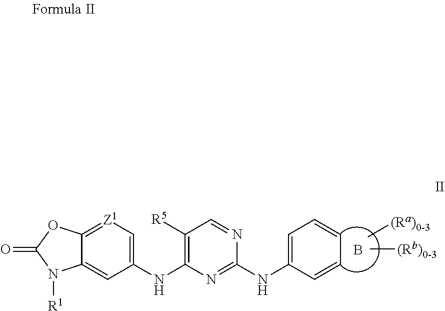
or a salt, solvate, N-oxide or prodrug thereof, wherein
ring B, together with the two phenyl ring atoms to which it is attached, forms a 5, 6 or 7-membered ring, optionally containing 1, 2 or 3 heteroatoms independently selected from N(R^{c}), O and S;
each R^{a} is C₁₋₆alkyl; and
each R^{b} is independently for each occurrence ═O, —OR^{a}, haloC₁₋₃alkyloxy, —SR^{a}, —N(R^{c})₂, halo, —CF₃, —CN, —S(O)₂N(R^{c})₂, —S(O)₂R^{a}, —C(O)R^{a}, —CO₂R^{a}, —C(O)N(R^{c})₂, —N(R^{a})—S(O)₂R^{a} or —C(R^{a})₂—N(R^{c})₂.

5. The method of claim 3, wherein the compound is:
N2-(3,4,5-trimethyl)phenyl-5-methyl-N4-(2-oxo-2,3-dihydro-1,3-benzoxazol-5-yl)-2,4-pyrimidinediamine, or a pharmaceutically acceptable salt thereof;
5-methyl-N4-[3-(phosphonooxy)methyl-2-oxo-2,3-dihydro-1,3-benzoxazol-5-yl]-N2-(3,4,5-trimethyl)phenyl-2,4-pyrimidinediamine, or a pharmaceutically acceptable salt thereof;
5-methyl-N4-[3-(phosphonooxy)methyl-2-oxo-2,3-dihydro-1,3-benzoxazol-5-yl]-N2-(3,4,5-trimethyl)phenyl-2,4-pyrimidinediamine bis-sodium salt;
N2-(3-methoxy-5-trifluoromethyl)phenyl-5-methyl-N4-[3-(phosphonooxy)methyl-2-oxo-2,3-dihydro-1,3-benzoxazol-5-yl]-2,4-pyrimidinediamine, or a pharmaceutically acceptable salt thereof;
N2-(3-methoxy-5-trifluoromethyl)phenyl-5-methyl-N4-[3-(phosphonooxy)methyl-2-oxo-2,3-dihydro-1,3-benzoxazol-5-yl]-2,4-pyrimidinediamine bis-sodium salt;
5-(2-(3-methoxy-4,5-dimethylphenylamino)-5-methylpyrimidin-4-ylamino)benzo[d]oxazol-2(3H)-one, or a pharmaceutically acceptable salt thereof;
(5-(2-(4-fluoro-3-methoxy-5-methylphenylamino)-5-methylpyrimidin-4-ylamino)-2-oxobenzo[d]oxazol-3(2H)-yl)methyl dihydrogen phosphate;
5-(2-(4-fluoro-3-methoxy-5-methylphenylamino)-5-methylpyrimidin-4-ylamino)benzo[d]oxazol-2(3H)-one, or a pharmaceutically acceptable salt thereof; or sodium (5-(2-(4-fluoro-3-methoxy-5-methylphenylamino)-5-methylpyrimidin-4-ylamino)-2-oxobenzo[d]oxazol-3(2H)-yl)methyl phosphate.

6. The method of claim 1, wherein the compound is a pyrimidine diamine compound according to Formula III 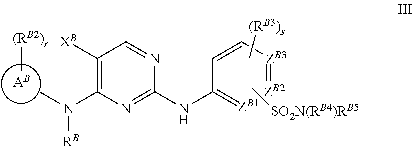
or a salt, solvate, N-oxide or prodrug thereof, wherein:
X^{B} is alkyl, alkoxy, amino, carboxyl, carboxyl ester, cyano, halo, nitro, alkenyl, or alkynyl;
R^{B} is hydrogen, alkyl, alkenyl, alkynyl, or cycloalkyl;
ring A^{B} is aryl, heteroaryl, cycloalkyl, cycloalkenyl or heterocyclic, wherein ring A is not indolyl or benzimidazolyl;
r is 0, 1, 2 or 3;
each R^{B2} independently is alkyl, alkoxy, amino, aryl, aryloxy (i.e. aryl-O—), cyano, cycloalkyl, cycloalkoxy, heteroaryl, heteroaryloxy, heterocyclic, heterocyclyloxy, aminoacyl, carboxyl, carboxyl ester, carbonate ester, sulfonyl, oxo, nitro or halo, preferably alkoxy;
Z^{B1}, Z^{B2}, and Z^{B3} each independently is carbon or nitrogen, wherein if Z^{B1} is nitrogen then Z^{B2} and Z^{B3} are carbon, if Z^{B2} is nitrogen then Z^{B1} and Z^{B3} are carbon, and if Z^{B3} is nitrogen then Z^{B1} and Z^{B2} are carbon, wherein if Z^{B1}, Z^{B2}, or Z^{B3} is nitrogen then SO₂R^{B4}R^{B5} is not attached to the nitrogen;
s is 0, 1, 2 or 3;
each R^{B3} independently is hydrogen, alkyl, alkoxy, or cycloalkyl, halo, or heterocyclic;
each of R^{B4} and R^{B5} independently is hydrogen, alkyl, acyl or M⁺, wherein M⁺ is a metal counterion selected from K⁺, Na⁺, Li⁺ or ⁺N(R⁶)₄, wherein R^{B6} is hydrogen or alkyl, and the nitrogen of SO₂NR^{B4}R^{B5} is N⁻;
or R^{B4} or R^{B5} is a divalent counterion selected from Ca²⁺, Mg²+, and Ba²⁺, and the nitrogen of SO₂NR^{B4}R^{B5} is N⁻.

7. The method of claim 6, wherein the compound is

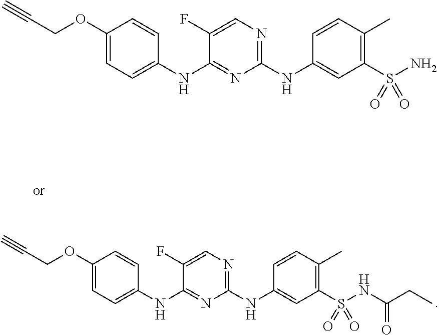 [SMILES: C#CCOc1ccc(Nc2nc(Nc3ccc(C)c(S(N)(=O)=O)c3)ncc2F)cc1 ; C#CCOc1ccc(Nc2nc(Nc3ccc(C)c(S(=O)(=O)NC(=O)CC)c3)ncc2F)cc1]

8. The method of claim 1, wherein the compound is a pyrazole compound according to Formula IV 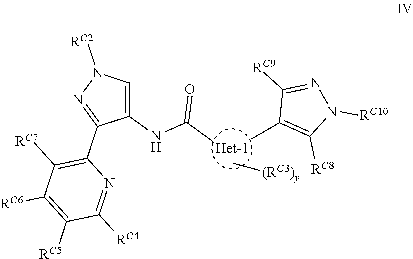
or a salt, solvate and/or N-oxide thereof, wherein:
Het-1 is 5-membered heteroaryl;
y is from 1 to 2;
R^{C2} is H, aliphatic, heteroaliphatic, heterocycloaliphatic, aryl, amide, heterocyclyl or araliphatic;
each R^{C3} independently is H or aliphatic;
R^{C4}, R^{C5}, R^{C6} and C₇ are each independently H, aliphatic, heteroaliphatic, alkoxy, heterocyclyl, aryl, araliphatic, —O-heterocyclyl, hydroxyl, haloalkyl, halogen, nitro, cyano, carboxyl, carboxyl ester, acyl, amide, amino, sulfonyl, sulfonamide, sulfanyl or sulfinyl;
R^{C8} and R^{C9} are each independently H, aliphatic, heteroaliphatic, aryl, heterocyclyl, sulfonyl, nitro, halogen, haloalkyl, carboxyl ester, cyano or amino; and
R^{C10} is H, aliphatic, alkoxy, heteroaliphatic, carboxyl ester, araliphatic, NO₂, CN, OH, haloalkyl, acyl, alkyl phosphate or alkylphosphonate.

9. The method of claim 8, wherein:
Het-1 is thiazolyl or furanyl;
R^{C10} is H, alkyl, alkyl phosphate or alkyl phosphonate;
each of R^{C4}, R^{C6}, and R^{C7} independently is H, halo, alkyl or haloalkyl; or a combination thereof

10. The method of claim 8, wherein:
each of R^{C4}, R^{C6}, and R^{C7} independently is H or F;
R^{C5} is H, F, CF₃, methoxy, —O—CH₂C(CH₃)₂OH, morpholin-4-yl, 1-methylpiperidin-4-yl, or —O— (oxetan-3-yl); or
a combination thereof.

11. The method of claim 8, wherein the compound is a pyrazole compound according to Formula V or Formula VI 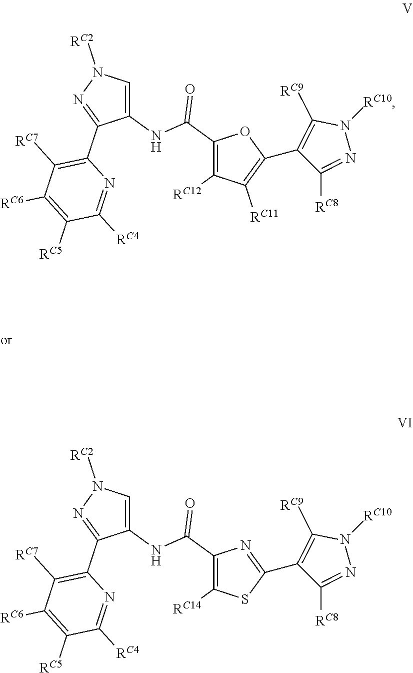
or a salt, solvate and/or N-oxide thereof, wherein:
each of R^{C11} and R^{C12} independently is H or aliphatic; and
R^{C14} is H or aliphatic.

12. The method of claim 8, wherein the pyrazole compound is 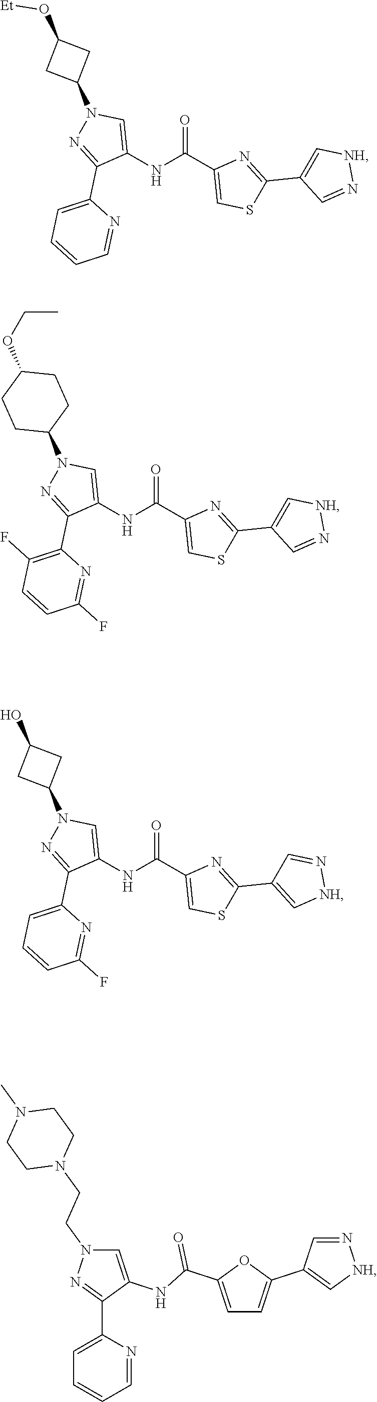 [SMILES: CCO[C@H]1C[C@@H](n2cc(NC(=O)c3csc(-c4cn[nH]c4)n3)c(-c3ccccn3)n2)C1 ; CCO[C@H]1CC[C@H](n2cc(NC(=O)c3csc(-c4cn[nH]c4)n3)c(-c3nc(F)ccc3F)n2)CC1 ; O=C(Nc1cn([C@H]2C[C@@H](O)C2)nc1-c1cccc(F)n1)c1csc(-c2cn[nH]c2)n1 ; CN1CCN(CCn2cc(NC(=O)c3ccc(-c4cn[nH]c4)o3)c(-c3ccccn3)n2)CC1] 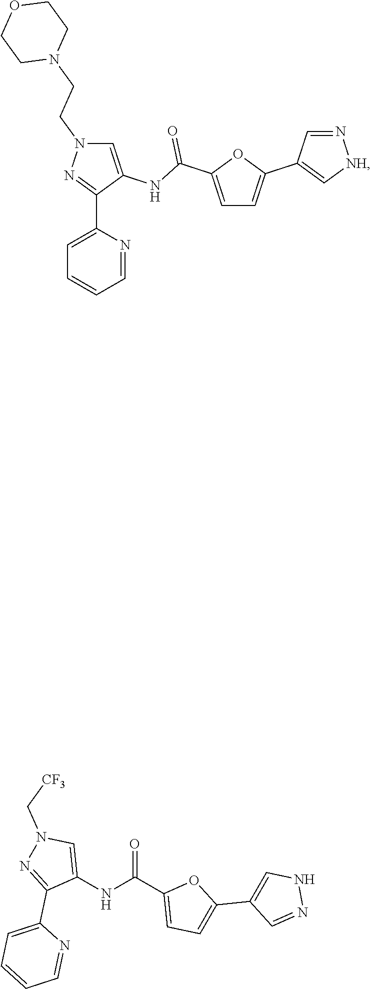 [SMILES: O=C(Nc1cn(CCN2CCOCC2)nc1-c1ccccn1)c1ccc(-c2cn[nH]c2)o1 ; O=C(Nc1cn(CC(F)(F)F)nc1-c1ccccn1)c1ccc(-c2cn[nH]c2)o1]
or a pharmaceutically acceptable salt thereof.

13. The method of claim 1, wherein the compound is selected from:
I-1 N4-(benzo[d]oxazol-2(3H)-on-5-yl)-N2-(3-formylphenyl)-5-methylpyrimidine-2,4-diamine;
I-2 N4-(benzo[d]oxazol-2(3H)-on-5-yl)-N2-(3-aminocarbonylphenyl)-5-methylpyrimidine-2,4-diamine;
I-3 N4-(benzo[d]oxazol-2(3H)-on-5-yl)-N2-(4-aminocarbonylphenyl)-5-methylpyrimidine-2,4-diamine;
I-4 N4-(benzo[d]oxazol-2(3H)-on-5-yl)-N2-(4-formylphenyl)-5-methylpyrimidine-2,4-diamine;
I-5 N4-(benzo[d]oxazol-2(3H)-on-5-yl)-N2-(3-methyl-4-(1,5,7-trimethyl-3,7-diazabicyclo [3.3.1]nonan-3-yl)phenyl)-5-methylpyrimidine-2,4-diamine;
I-6 N4-(benzo[d]oxazol-2(3H)-on-5-yl)-N2-(3-fluoro-4-(1,5,7-trimethyl-3,7-diazabicyclo [3.3.1]nonan-3-yl)phenyl)-5-methylpyrimidine-2,4-diamine;
I-7 N4-(3-n-propylbenzo[d]oxazol-2(3H)-on-5-yl)-N2-((3-methylsulfonyl)phenyl)-5-methylpyrimidine-2,4-diamine;
I-9 N4-(3-isopropylbenzo[d]oxazol-2(3H)-on-5-yl)-N2-((3-methylsulfonyl)phenyl)-5-methylpyrimidine-2,4-diamine;
I-16 N4-(benzo[d]oxazol-2(3H)-on-5-yl)-N2-(3-methylsulfonyl)phenyl)-5-methylpyrimidine-2,4-diamine;
I-17 N4-(benzo[d]oxazol-2(3H)-on-5-yl)-N2-(4-methylsulfonyl)phenyl)-5-methylpyrimidine-2,4-diamine;
I-20 N4-(benzo[d]oxazol-2(3H)-on-5-yl)-N2-(3-methylsulfonyl)phenyl)-5-fluoropyrimidine-2,4-diamine;
I-21 N4-(benzo[d]oxazol-2(3H)-on-5-yl)-N2-(4-methylsulfonyl)phenyl)-5-fluoropyrimidine-2,4-diamine;
I-22 N4-(benzimidazolin-2-on-5-yl)-N2-(3-methylsulfonyl)phenyl-5-methylpyrimidine-2,4-diamine;
I-23 N4-(benzimidazolin-2-on-5-yl)-N2-(4-methylsulfonyl)phenyl-5-fluoropyrimidine-2,4-diamine;
I-26 N4-(benzo[d]oxazol-2(3H)-on-5-yl)-N2-(3-cyanophenyl)-5-methylpyrimidine-2,4-diamine;
I-27 N4-(benzo[d]oxazol-2(3H)-on-5-yl)-N2-(4-cyanophenyl)-5-methylpyrimidine-2,4-diamine;
I-28 N4-(benzimidazolin-2-on-5-yl)-N2-(3-cyanophenyl)-5-methylpyrimidine-2,4-diamine;
I-29 N4-(benzimidazolin-2-on-5-yl)-N2-(4-cyanophenyl)-5-methylpyrimidine-2,4-diamine;
I-33 N4-(3-phosphorylmethylbenzo[d]oxazol-2(3H)-on-5-yl)-N2-((3-methylsulfonyl)phenyl)-5-methylpyrimidine-2,4-diamine;
I-36 N4-(1,3-dimethylbenzimidazolin-2-on-5-yl)-N2-((3-methylsulfonyl)phenyl)-5-methylpyrimidine-2,4-diamine;
I-41 N4-(1,3-dimethylbenzimidazolin-2-on-5-yl)-N2-((3-methylsulfonyl)phenyl)-5-fluoropyrimidine-2,4-diamine;
I-44 N4-(benzo[d]oxazol-2(3H)-on-5-yl)-N2-(3-cyanophenyl)-5-fluoropyrimidine-2,4-diamine;
I-45 N4-(benzo[d]oxazol-2(3H)-on-5-yl)-N2-(4-cyanophenyl)-5-fluoropyrimidine-2,4-diamine;
I-46 N4-(benzo[d]oxazol-2(3H)-on-5-yl)-N2-((3-morpholinyl)phenyl)-5-methylpyrimidine-2,4-diamine;
I-47 N4-(benzo[d]oxazol-2(3H)-on-5-yl)-N2-((3-morpholinyl)phenyl)-5-fluoropyrimidine-2,4-diamine;
I-48 N4-(benzo[d]oxazol-2(3H)-on-6-yl)-N2-((3-morpholinyl)phenyl)-5-methylpyrimidine-2,4-diamine;
I-49 N4-(3-methylbenzo[d]oxazol-2(3H)-on-6-yl)-N2-((3-morpholinyl)phenyl)-5-methylpyrimidine-2,4-diamine;
I-59 N2-((3-methylsulfonyl)phenyl)-N4-(3-methyl-2-oxo-2,3-dihydrobenzo[d]oxazol-6-yl)-5-fluoropyrimidine-2,4-diamine;
I-60 N2-(4-methylsulfonyl)phenyl)-N4-(3-methyl-2-oxo-2,3-dihydrobenzo[d]oxazol-6-yl)-5-fluoropyrimidine-2,4-diamine;
I-65 N2-((3-methylsulfonyl)phenyl)-N4-(3-methyl-2-oxo-2,3-dihydrobenzo[d]oxazol-6-yl)-5-methylpyrimidine-2,4-diamine;
I-66 N2-((4-methylsulfonyl)phenyl)-N4-(3-methyl-2-oxo-2,3-dihydrobenzo[d]oxazol-6-yl)-5-methylpyrimidine-2,4-diamine;
I-69 N2-((3-methylsulfonyl)phenyl)-N4-(3-methyl-2-oxo-2,3-dihydrobenzo[d]oxzo1-5-yl)-5-methylpyrimidine-2,4-diamine;
I-70 N2-((4-methylsulfonyl)phenyl)-N4-(3-methyl-2-oxo-2,3-dihydrobenzo[d]oxazol-5-yl)-5-methylpyrimidine-2,4-diamine;
I-77 N2-((3-methylsulfonyl)phenyl)-N4-(3-methyl-2-oxo-2,3-dihydrobenzo[d]oxazol-5-yl)-5-fluoropyrimidine-2,4-diamine;
I-78 N2-((4-methylsulfonyl)phenyl)-N4-(3-methyl-2-oxo-2,3-dihydrobenzo[d]oxazol-5-yl)-5-fluoropyrimidine-2,4-diamine;
I-100 N2-((3-methylsulfonyl)phenyl)-N4-(2-oxo-2,3-dihydrobenzo[d]oxazol-6-yl)-5-methylpyrimidine-2,4-diamine;
I-101 N2-((4-methylsulfonyl)phenyl)-N4-(2-oxo-2,3-dihydrobenzo[a]oxazol-6-yl)-5-methylpyrimidine-2,4-diamine;
I-106 N4-(benzoxazolin-2-on-5-yl)-N2-(3-trifluoromethoxyphenyl)-5-methylpyrimidine-2,4-diaminetrifluoroacetate salt;
I-107 N4-(benzoxazolin-2-on-5-yl)-N2-(3-trifluoromethoxyphenyl)-5-fluoropyrimidine-2,4-diamine trifluoroacetate salt;
I-108 N4-(benzoxazolin-2-on-5-yl)-N2-(4-trifluoromethoxyphenyl)-5-methylpyrimidine-2,4-diamine trifluoroacetate salt;
I-109 N4-(benzoxazolin-2-on-5-yl)-N2-(4-trifluoromethoxyphenyl)-5-fluoropyrimidine-2,4-diaminetrifluoroacetate salt;
I-110 N4-(benzoxazolin-2-on-5-yl)-N2-[3-trifluoromethyl-4-(4-ethylpiperazin-1-yl)phenyl]-5-methylpyrimidine-2,4-diamine;
I-111 N4-(benzoxazolin-2-on-5-yl)-N2-[3-methyl-4-(4-methylpiperazin-1-yl)phenyl]-5-methylpyrimidine-2,4-diamine;
I-115 N4-(benzoxazolin-2-on-5-yl)-N2-(3,4,5-trimethoxyphenyl)-5-fluoropyrimidine-2,4-diamine;
I-116 N2-(3-(difluoromethoxy)-4-methoxyphenyl)-N4-(benzo[d]oxazol-2 (3H)-on-5-yl)-5-methylpyrimidine-2,4-diamine;
I-117 N4-(benzo[d]oxazol-2(3H)-on-5-yl)-N2-(4-trifluoromethylsulfonyl)phenyl-5-methylpyrimidine-2,4-diamine;
I-118 N4-(benzo[d]oxazol-2 (3H)-on-5-yl)-N2-(3-trifluoromethyl sulfonyl)phenyl-5-methylpyrimidine-2,4-diamine;
I-119 N4-(benzo[d]oxazol-2(3H)-on-5-yl)-N2-(3,4,5-trimethoxy)phenyl-5-methylpyrimidine-2,4-diamine;
I-120 N4-(benzo[d]oxazol-2(3H)-on-5-yl)-N2-((4-(1,4-diazabicyclo[3.2.2]nonan-4-yl)-3-methyl)phenyl)-5-methylpyrimidine-2,4-diamine;
I-121 N4-(benzo[d]oxazol-2(3H)-on-5-yl)-N2-(4-(8-methyl-8-azabicyclo[3.2.1]octan-3-ylamino)phenyl)-5-methylpyrimidine-2,4-diamine;
I-122 N4-(benzo[d]oxazol-2(3H)-on-5-yl)-N2-((4-(dihydro-1H-pyrido [1,2-a]pyrazin-2 (6H,7H, 8H,9H,9aH)-yl)-3-methyl)phenyl)-5-methylpyrimidine-2,4-diamine;
I-123 N4-(benzo[d]oxazol-2(3H)-on-5-yl)-N2-(4-(8-methyl-2,8-diazabicyclo[3.2.1]octan-2-yl)phenyl)-5-methylpyrimidine-2,4-diamine;
I-124 N4-(benzo[d]oxazolin-2(3H)-one-5-yl)-5-methyl —N2-[3-(morpholin-4-yl)-4-trifluoromethoxyphenyl]-2,4-pyrimidinediamine;
I-125 N4-(benzo[d]oxazolin-2(3H)-on-5-yl)-N2-[3-trifluoromethyl-2-(4-methylpiperazin-1-yl)pyridin-5-yl]-5-methylpyrimidine-2,4-diamine;
I-126 4-[5-Methyl-4-(2-oxo-2,3-dihydro-benzooxazol-5-ylamino)-pyrimidin-2-ylamino]-benzoic acid;
I-127 N-(2-Diethylamino-ethyl)-4-[5-methyl-4-(2-oxo-2,3-dihydro-benzooxazol-5-ylamino)-pyrimidin-2-ylamino]-benzamide;
I-128 5-{2-[4-(3-Diethylamino-pyrrolidine-1-carbonyl)-phenylamino]-5-methyl-pyrimidin-4-ylamino}-3H-benzooxazol-2-one;
I-129 5-[2-(4-Acetyl-phenylamino)-5-methyl-pyrimidin-4-ylamino]-3H-benzooxazol-2-one;
I-130 5-[2-(3-Acetyl-phenylamino)-5-methyl-pyrimidin-4-ylamino]-3H-benzooxazol-2-one;
I-131 2-Methyl-5-[5-methyl-4-(2-oxo-2,3-dihydro-benzooxazol-5-ylamino)-pyrimidin-2-ylamino]-benzonitrile;
I-132 N,N-Dimethyl-4-[5-methyl-4-(2-oxo-2,3-dihydro-benzooxazol-5-ylamino)-pyrimidin-2-ylamino]-benzamide;
I-133 N-Methyl-4-[5-methyl-4-(2-oxo-2,3-dihydro-benzooxazol-5-ylamino)-pyrimidin-2-ylamino]-benzamide;
I-134 N-Cyclopropyl-4-[5-methyl-4-(2-oxo-2,3-dihydro-benzooxazol-5-ylamino)-pyrimidin-2-ylamino]-benzamide formate salt
I-135 4-[5-Methyl-4-(2-oxo-2,3-dihydro-benzooxazol-5-ylamino)-pyrimidin-2-ylamino]-N-phenyl-benzamide;
I-136 4-[5-Methyl-4-(2-oxo-2,3-dihydro-benzooxazol-5-ylamino)-pyrimidin-2-ylamino]-2-pyrrolidin-1-yl-benzamide;
I-137 N-Ethyl-4-[5-methyl-4-(2-oxo-2,3-dihydro-benzooxazol-5-ylamino)-pyrimidin-2-ylamino]-benzamide;
I-138 N-Cyclobutyl-4-[5-methyl-4-(2-oxo-2,3-dihydro-benzooxazol-5-ylamino)-pyrimidin-2-ylamino]-benzamide formic acid salt;
I-139 N-Isopropyl-4-[5-methyl-4-(2-oxo-2,3-dihydro-benzooxazol-5-ylamino)-pyrimidin-2-ylamino]-benzamide;
I-140 N-Cyclopropyl-4-[5-methyl-4-(2-oxo-2,3-dihydro-benzooxazol-5-ylamino)-pyrimidin-2-ylamino]-benzamide formate salt;
I-141 2-Chloro-4-[5-methyl-4-(2-oxo-2,3-dihydro-benzooxazol-5-ylamino)-pyrimidin-2-ylamino]-benzamide;
I-142 N-Cyclopropyl-4-[5-methyl-4-(2-oxo-2,3-dihydro-benzooxazol-5-ylamino)-pyrimidin-2-ylamino]-benzamide trifluoroacetic acid salt;
I-143 N-Cyclopropyl-4-[5-methyl-4-(2-oxo-2,3-dihydro-benzooxazol-5-ylamino)-pyrimidin-2-ylamino]-benzamide;
I-144 N-Cyclobutyl-4-[5-methyl-4-(2-oxo-2,3-dihydro-benzooxazol-5-ylamino)-pyrimidin-2-ylamino]-benzamide;
I-145 4-[5-Methyl-4-(2-oxo-3-propionyl-2,3-dihydro-benzooxazol-5-ylamino)-pyrimidin-2-ylamino]-benzamide;
I-146 di-tert-butyl (5-(2-(4-carbamoylphenylamino)-5-methylpyrimidin-4-ylamino)-2-oxobenzo[d]oxazol-3(2H)-yl)methyl phosphate;
I-147 (5-(2-(4-carbamoylphenylamino)-5-methylpyrimidin-4-ylamino)-2-oxobenzo[d]oxazol-3(2H)-yl)methyl dihydrogen phosphate;
I-148 sodium (5-(2-(4-carbamoylphenylamino)-5-methylpyrimidin-4-ylamino)-2-oxobenzo[d]oxazol-3(2H)-yl)methyl phosphate;
I-150 (5-(2-(4-(cyclobutylcarbamoyl)phenylamino)-5-methylpyrimidin-4-ylamino)-2-oxobenzo[d]oxazol-3(2H)-yl)methyl dihydrogen phosphate;
I-151 sodium (5-(2-(4-(cyclobutylcarbamoyl)phenylamino)-5-methylpyrimidin-4-ylamino)-2-oxobenzo[d]oxazol-3(2H)-yl)methyl phosphate;
I-152 di-tert-butyl (5-(2-(4-(cyclobutylcarbamoyl)phenylamino)-5-methylpyrimidin-4-ylamino)-2-oxobenzo[d]oxazol-3(2H)-yl)methyl phosphate;
I-153 5-[2-(4-Chloro-3-trifluoromethyl-phenylamino)-5-methyl-pyrimidin-4-ylamino]-3H-benzooxazol-2-one;
I-154 5-[5-Methyl-2-(4-methyl-3-trifluoromethyl-phenylamino)-pyrimidin-4-ylamino]-3H-benzooxazol-2-one;
I-155 5-[5-Methyl-2-(4-methylsulfanyl-3-trifluoromethyl-phenylamino)-pyrimidin-4-ylamino]-3H-benzooxazol-2-one;
I-156 4-[5-Methyl-4-(2-oxo-2,3-dihydro-benzooxazol-5-ylamino)-pyrimidin-2-ylamino]-2-(4-methyl-piperidin-1-yl)-benzamide;
I-157 5-[2-(3-Cyclopentanesulfonyl-phenylamino)-5-methyl-pyrimidin-4-ylamino]-3H-benzooxazol-2-one;
I-158 5-[5-Methyl-2-(3-trifluoromethyl-phenylamino)-pyrimidin-4-ylamino]-3H-benzooxazol-2-one;
I-159 2-Methyl-4-[5-methyl-4-(2-oxo-2,3-dihydro-benzooxazol-5-ylamino)-pyrimidin-2-ylamino]-benzoic acid methyl ester;
I-160 5-[5-Methyl-2-(4-trifluoromethyl-phenylamino)-pyrimidin-4-ylamino]-3H-benzooxazol-2-one;
I-161 5-[5-Methyl-2-(4-trifluoromethoxy-3-trifluoromethyl-phenylamino)-pyrimidin-4-ylamino]-3H-benzooxazol-2-one;
I-162 5-[2-(3-Fluoro-5-trifluoromethyl-phenylamino)-5-methyl-pyrimidin-4-ylamino]-3H-benzooxazol-2-one triflouroacetate salt;
I-163 5-[2-(4-(4-Fluoro-3-trifluoromethoxy-phenylamino)-5-methyl-pyrimidin-4-ylamino]-3H-benzooxazol-2-one;
I-164 5-[5-Methyl-2-(4-methyl-3-trifluoromethyl-phenylamino)-pyrimidin-4-ylamino]-3H-benzooxazol-2-one trifluoracetic acid salt;
I-165 5-{2-[4-(2-Methoxy-ethoxy)-3-trifluoromethyl-phenylamino]-5-methyl-pyrimidin-4-ylamino}-3H-benzooxazol-2-one;
I-166 5-[2-(4-(4-Isopropyl-3-methyl-phenylamino)-5-methyl-pyrimidin-4-ylamino]-3H-benzooxazol-2-one;
I-167 5-[2-(3-Chloro-4-trifluoromethyl-phenylamino)-5-methyl-pyrimidin-4-ylamino]-3H-benzooxazol-2-one;
I-168 5-[2-(4-Ethoxy-3-trifluoromethyl-phenylamino)-5-methyl-pyrimidin-4-ylamino]-3H-benzooxazol-2-one;
I-169 5-[2-(3,5-B is-trifluoromethyl-phenylamino)-5-methyl-pyrimidin-4-ylamino]-3H-benzooxazol-2-one;
I-170 2-Methyl-4-[5-methyl-4-(2-oxo-2,3-dihydro-benzooxazol-5-ylamino)-pyrimidin-2-ylamino]-benzoic acid;
I-171 N-Ethyl-2-methyl-4-[5-methyl-4-(2-oxo-2,3-dihydro-benzooxazol-5-ylamino)-pyrimidin-2-ylamino]-benzamide;
I-172 5-[2-(4-Chloro-phenylamino)-5-methyl-pyrimidin-4-ylamino]-3H-benzooxazol-2-one;
I-173 5-[2-(3-Chloro-phenylamino)-5-methyl-pyrimidin-4-ylamino]-3H-benzooxazol-2-one;
I-174 5-(5-Methyl-2-phenylamino-pyrimidin-4-ylamino)-3H-benzooxazol-2-one;
I-175 5-[2-(3-Bromo-phenylamino)-5-methyl-pyrimidin-4-ylamino]-3H-benzooxazol-2-one;
I-176 5-[2-(4-Chloro-2,5-dimethyl-phenylamino)-5-methyl-pyrimidin-4-ylamino]-3H-benzooxazol-2-one;
I-177 N-{4-[5-Methyl-4-(2-oxo-2,3-dihydro-benzooxazol-5-ylamino)-pyrimidin-2-ylamino]-2-trifluoromethyl-phenyl}-acetamide;
I-178 5-[2-(3,4-Dimethyl-phenylamino)-5-methyl-pyrimidin-4-ylamino]-3H-benzooxazol-2-one;
I-179 5-[2-(4-Cyclohexylmethoxy-3-trifluoromethyl-phenylamino)-5-methyl-pyrimidin-4-ylamino]-3H-benzooxazol-2-one;
I-180 5-[2-(4-Chloro-3-trifluoromethoxy-phenylamino)-5-methyl-pyrimidin-4-ylamino]-3H-benzooxazol-2-one;
I-181 5-[2-(4-Chloro-3-methoxy-phenylamino)-5-methyl-pyrimidin-4-ylamino]-3H-benzooxazol-2-one;
I-182 5-[2-(4-Chloro-3-ethoxy-phenylamino)-5-methyl-pyrimidin-4-ylamino]-3H-benzooxazol-2-one;
I-183 5-[2-(4-Fluoro-3-methoxy-phenylamino)-5-methyl-pyrimidin-4-ylamino]-3H-benzooxazol-2-one;
I-184 5-[2-(3,5-Dichloro-phenylamino)-5-methyl-pyrimidin-4-ylamino]-3H-benzooxazol-2-one;
I-185 5-[2-(3-Bromo-5-chloro-phenylamino)-5-methyl-pyrimidin-4-ylamino]-3H-benzooxazol-2-one;
I-186 5-[2-(3-Chloro-5-fluoro-phenylamino)-5-methyl-pyrimidin-4-ylamino]-3H-benzooxazol-2-one;
I-187 3-Chloro-5-[5-methyl-4-(2-oxo-2,3-dihydro-benzooxazol-5-ylamino)-pyrimidin-2-ylamino]-benzonitrile;
I-188 5-[2-(4-Bromo-3-trifluoromethyl-phenylamino)-5-methyl-pyrimidin-4-ylamino]-3H-benzooxazol-2-one;
I-189 5-[2-(3-Bromo-5-trifluoromethyl-phenylamino)-5-methyl-pyrimidin-4-ylamino]-3H-benzooxazol-2-one;
I-190 N-Cyclobutyl-2-methyl-4-[5-methyl-4-(2-oxo-2,3-dihydro-benzooxazol-5-ylamino)-pyrimidin-2-ylamino]-benzamide;
I-191 5-{2-[3-Chloro-4-(2-morpholin-4-yl-ethoxy)-phenylamino]-5-methyl-pyrimidin-4-ylamino}-3H-benzooxazol-2-one;
I-192 5-{5-Methyl-2-[4-(2-morpholin-4-yl-ethoxy)-phenylamino]-pyrimidin-4-ylamino}-3H-benzooxazol-2-one;
I-193 5-[2-(2,4-Difluoro-5-methoxy-phenylamino)-5-methyl-pyrimidin-4-ylamino]-3H-benzooxazol-2-one;
I-194 5-[2-(3-Chloro-4-ethoxy-phenylamino)-5-methyl-pyrimidin-4-ylamino]-3H-benzooxazol-2-one;
I-195 5-[2-(4-Cyclobutylmethoxy-phenylamino)-5-methyl-pyrimidin-4-ylamino]-3H-benzooxazol-2-one;
I-196 5-[2-(4-Isobutoxy-phenylamino)-5-methyl-pyrimidin-4-ylamino]-3H-benzooxazol-2-one;
I-197 5-{5-Methyl-2-[4-(3-methyl-butoxy)-phenylamino]-pyrimidin-4-ylamino}-3H-benzooxazol-2-one;
I-198 5-[2-(3-Chloro-4-trifluoromethyl-phenylamino)-5-methyl-pyrimidin-4-ylamino]-3H-benzooxazol-2-one trifluoroacetic acid salt;
I-199 5-[2-(3-Fluoro-5-methyl-phenylamino)-5-methyl-pyrimidin-4-ylamino]-3H-benzooxazol-2-one;
I-200 5-[2-(2,4-Difluoro-3-methoxy-phenylamino)-5-methyl-pyrimidin-4-ylamino]-3H-benzooxazol-2-one;
I-201 5-(2-(4-(1-(azetidin-1-yl)ethyl)phenylamino)-5-methylpyrimidin-4-ylamino)benzo[d]oxazol-2(3H)-one;
I-202 5-(2-(4-(1-(cyclopropylamino)ethyl)phenylamino)-5-methylpyrimidin-4-ylamino)benzo[d]oxazol-2(3H)-one;
I-203 5-(5-methyl-2-(4-(1-(pyrrolidin-1-yl)ethyl)phenylamino)pyrimidin-4-ylamino)benzo[d]oxazol-2(3H)-one;
I-204 5-(5-methyl-2-(4-(1-morpholinoethyl)phenylamino)pyrimidin-4-ylamino)benzo[d]oxazol-2(3H)-one;
I-205 5-(2-(4-(1-(3-(diethylamino)pyrrolidin-1-yl)ethyl)phenylamino)-5-methylpyrimidin-4-ylamino)benzo[d]oxazol-2(3H)-one;
I-206 5-(2-(4-(1-(benzylamino)ethyl)phenylamino)-5-methylpyrimidin-4-ylamino)benzo[d]oxazol-2(3H)-one;
I-207 5-(2-(4-(1-(isopropylamino)ethyl)phenylamino)-5-methylpyrimidin-4-ylamino)benzo[d]oxazol-2(3H)-one;
I-208 5-(5-methyl-2-(3-(1-(propylamino)ethyl)phenylamino)pyrimidin-4-ylamino)benzo[d]oxazol-2(3H)-one;
I-209 5-(2-(3-(1-(isopropylamino)ethyl)phenylamino)-5-methylpyrimidin-4-ylamino)benzo[d]oxazol-2(3H)-one;
I-210 5-(2-(3-(1-(isopropylamino)ethyl)phenylamino)-5-methylpyrimidin-4-ylamino)benzo[d]oxazol-2(3H)-one;
I-211 5-(2-(3-(1-(azetidin-1-yl)ethyl)phenylamino)-5-methylpyrimidin-4-ylamino)benzo[d]oxazol-2(3H)-one;
I-212 5-(5-methyl-2-(3-(1-(pyrrolidin-1-yl)ethyl)phenylamino)pyrimidin-4-ylamino)benzo[d]oxazol-2(3H)-one;
I-213 5-(2-(3-(1-(benzylamino)ethyl)phenylamino)-5-methylpyrimidin-4-ylamino)benzo[d]oxazol-2(3H)-one;
I-214 5-(2-(3-(1-(3-(diethylamino)pyrrolidin-1-yl)ethyl)phenylamino)-5-methylpyrimidin-4-ylamino)benzo[d]oxazol-2(3H)-one;
I-215 5-(5-methyl-2-(3-(1-(piperidin-1-yl)ethyl)phenylamino)pyrimidin-4-ylamino)benzo[d]oxazol-2(3H)-one;
I-216 5-(2-(3-(1-(diethylamino)ethyl)phenylamino)-5-methylpyrimidin-4-ylamino)benzo[d]oxazol-2(3H)-one;
I-217 5-(5-methyl-2-(3-(1-morpholinoethyl)phenylamino)pyrimidin-4-ylamino)benzo[d]oxazol-2(3H)-one;
I-218 N-cyclobutyl-4-(5-methyl-4-(2-oxo-2,3-dihydrobenzo[d]oxazol-5-ylamino)pyrimidin-2-ylamino)-2-(trifluoromethyl)benzamide;
I-219 4-(5-methyl-4-(2-oxo-2,3-dihydrobenzo[d]oxazol-5-ylamino)pyrimidin-2-ylamino)—N-phenyl-2-(trifluoromethyl)benzamide;
I-220 N-cyclopropyl-2-methoxy-4-(5-methyl-4-(2-oxo-2,3-dihydrobenzo[d]oxazol-5-ylamino)pyrimidin-2-ylamino)benzamide;
I-221 2-methoxy-4-(5-methyl-4-(2-oxo-2,3-dihydrobenzo[d]oxazol-5-ylamino)pyrimidin-2-ylamino)-N-phenylbenzamide;
I-222 4-(5-methyl-4-(2-oxo-2,3-dihydrobenzo[d]oxazol-5-ylamino)pyrimidin-2-ylamino)-2-(trifluoromethyl)benzoic acid;
I-223 N-cyclopropyl-4-(5-methyl-4-(2-oxo-2,3-dihydrobenzo[d]oxazol-5-ylamino)pyrimidin-2-ylamino)-2-(trifluoromethyl)benzamide;
I-224-(2-(3-isobutoxy-5-(trifluoromethyl)phenylamino)-5-methylpyrimidin-4-ylamino)benzo[d]oxazol-2(3H)-one;
I-225 5-(2-(3-(cyclopropylmethoxy)-5-(trifluoromethyl)phenylamino)-5-methylpyrimidin-4-ylamino)benzo[d]oxazol-2(3H)-one;
I-226 5-(2-(3-cyclobutoxy-5-(trifluoromethyl)phenylamino)-5-methylpyrimidin-4-ylamino)benzo[d]oxazol-2(3H)-one;
I-227 5-(2-(3-(cyclobutylmethoxy)-5-(trifluoromethyl)phenylamino)-5-methylpyrimidin-4-ylamino)benzo[d]oxazol-2(3H)-one;
I-228 5-(2-(3-deuteratedmethoxy-5-(trifluoromethyl)phenylamino)-5-methylpyrimidin-4-ylamino)benzo[d]oxazol-2(3H)-one;
I-229 5-(2-(3-acetyl-5-methoxyphenylamino)-5-methylpyrimidin-4-ylamino)benzo[d]oxazol-2(3H)-one;
I-230 5-(2-(3-chloro-4-fluoro-5-(trifluoromethyl)phenylamino)-5-methylpyrimidin-4-ylamino)benzo[d]oxazol2(3H)-one;
I-231 5-(2-(3-(1-(isopropylamino)ethyl)-5-methoxyphenylamino)-5-methylpyrimidin-4-ylamino)benzo[d]oxazol-2(3H)-one;
I-232 5-(2-(3-methoxy-5-(1-(propylamino)ethyl)phenylamino)-5-methylpyrimidin-4-ylamino)benzo[d]oxazol-2(3H)-one;
I-233 5-(2-(3-(1-(cyclopropylamino)ethyl)-5-methoxyphenylamino)-5-methylpyrimidin-4-ylamino)benzo[d]oxazol-2(3H)-one;
I-234 5-(2-(3-methoxy-5-(1-(pyrrolidin-1-yl)ethyl)phenylamino)-5-methylpyrimidin-4-ylamino)benzo[d]oxazol-2(3H)-one;
I-235 5-(2-(3-(1-(azetidin-1-yl)ethyl)-5-methoxyphenylamino)-5-methylpyrimidin-4-ylamino)benzo[d]oxazol-2(3H)-one;
I-236 5-(2-(3-methoxy-5-(1-(methylamino)ethyl)phenylamino)-5-methylpyrimidin-4-ylamino)benzo[d]oxazol-2(3H)-one;
I-237 5-(2-(3-(difluoromethyl)-5-methylphenylamino)-5-methylpyrimidin-4-ylamino)benzo[d]oxazol-2(3H)-one;
I-238 5-(2-(3-(fluoromethyl)-5-methylphenylamino)-5-methylpyrimidin-4-ylamino)benzo[d]oxazol-2(3H)-one;
I-239 5-(5-methyl-2-(4-methyl-3-(methylsulfonyl)phenylamino)pyrimidin-4-ylamino)benzo[d]oxazol-2(3H)-one;
I-240 5-(2-(3-fluoro-5-morpholinophenylamino)-5-methylpyrimidin-4-ylamino)benzo[d]oxazol-2(3H)-one;
I-241 5-(2-(3-fluoro-5-(4-methylpiperazin-1-yl)phenylamino)-5-methylpyrimidin-4-ylamino)benzo[d]oxazol-2(3H)-one;
I-242 5-(2-(4-fluoro-3-(methylsulfonyl)phenylamino)-5-methylpyrimidin-4-ylamino)benzo[d]oxazol-2(3H)-one;
I-244 7-methyl-5-(5-methyl-2-(3-(methylsulfonyl)phenylamino)pyrimidin-4-ylamino)benzo[d]oxazol-2(3H)-one;
I245 5-(2-(4-fluoro-3-(methylsulfonyl)phenylamino)-5-methylpyrimidin-4-ylamino)-7-methylbenzo[d]oxazol-2(3H)-one;
I-246 5-(5-methyl-2-(3-(pyrrolidine-1-carbonyl)phenylamino)pyrimidin-4-ylamino)benzo[d]oxazol-2(3H)-one;
I-247 5-(5-methyl-2-(4-(pyrrolidine-1-carbonyl)phenylamino)pyrimidin-4-ylamino)benzo[d]oxazol-2(3H)- one;
I-249 7-fluoro-5-(5-methyl-2-(3-(methylsulfonyl)phenylamino)pyrimidin-4-ylamino)benzo[d]oxazol-2(3H)-one;
I-250 7-fluoro-5-(2-(4-fluoro-3-(methylsulfonyl)phenylamino)-5-methylpyrimidin-4-ylamino)benzo[d]oxazol-2(3H)-one;
I-251 7-fluoro-5-(2-(3-methoxy-5-(trifluoromethyl)phenylamino)-5-methylpyrimidin-4-ylamino)benzo[d]oxazol-2(3H)-one;
I-252 3-methoxy-N,N-dimethyl-5-(5-methyl-4-(2-oxo-2,3-dihydrobenzo[d]oxazol-5-ylamino)pyrimidin-2-ylamino)benzamide;
I-253 5-(2-(3-methoxy-5-(pyrrolidine-1-carbonyl)phenylamino)-5-methylpyrimidin-4-ylamino)benzo [d]oxazol-2(3H)-one;
I-254 5-(2-(3-methoxy-5-(morpholine -4-carbonyl)phenylamino)-5-methylpyrimidin-4-ylamino)benzo [d]oxazol-2(3H)-one;
I-255 5-(2-(3-methoxy-5-(4-methylpiperazine-1-carbonyl)phenylamino)-5-methylpyrimidin-4-ylamino)benzo[d]oxazol-2(3H)-one;
I-256 5-(5-methyl-2-(3-(morpholine-4-carbonyl)phenylamino)pyrimidin-4-ylamino)benzo[d]oxazol-2(3H)-one;
I-257 5-(5-methyl-2-(4-(morpholine-4-carbonyl)phenylamino)pyrimidin-4-ylamino)benzo[d]oxazol-2(3H)-one;
I-258 5-(2-(4-methoxy-3-methylphenylamino)-5-methylpyrimidin-4-ylamino)benzo[d]oxazol-2(3H)-one;
I-259 5-(2-(3-methoxy-5-methylphenylamino)-5-methylpyrimidin-4-ylamino)benzo[d]oxazol-2(3H)-one;
I-260 2-methoxy-N,N-dimethyl-5-(5-methyl-4-(2-oxo-2,3-dihydrobenzo[d]oxazol-5-ylamino)pyrimidin-2-ylamino)benzamide;
I-261 5-(2-(4-methoxy-3-(pyrrolidine-1-carbonyl)phenylamino)-5-methylpyrimidin-4-ylamino)benzo[d]oxazol-2(3H)-one;
I-262 5-(2-(4-methoxy-3-(morpholine-4-carbonyl)phenylamino)-5-methylpyrimidin-4-ylamino)benzo[d]oxazol-2(3H)-one;
I-263 5-(2-(4-methoxy-3-(4-methylpiperazine-1-carbonyl)phenylamino)-5-methylpyrimidin-4-ylamino)benzo[d]oxazol-2(3H)-one;
I-264 5-(2-(3-methyl-4-trideuteromethoxyphenylamino)-5-methylpyrimidin-4-ylamino)benzo[d]oxazol-2(3H)-one;
I-265 5-(2-(3-chloro-4-methoxy-5-methylphenylamino)-5-methylpyrimidin-4-ylamino)benzo[d]oxazol-2(3H)-one;
I-266 5-(2-(3-methyl-5-trideuteromethoxyphenylamino)-5-methylpyrimidin-4-ylamino)benzo[d]oxazol-2(3H)-one;
I-267 2-methoxy-N,N-dimethyl-4-(5-methyl-4-(2-oxo-2,3-dihydrobenzo[d]oxazol-5-ylamino)pyrimidin-2-ylamino)benzamide;
I-268 5-(2-(3-methoxy-4-(pyrrolidine-1-carbonyl)phenylamino)-5-methylpyrimidin-4-ylamino)benzo[d]oxazol-2(3H)-one;
I-269 5-(2-(3-methoxy-4-(morpholine-4-carbonyl)phenylamino)-5-methylpyrimidin-4-ylamino)benzo[d]oxazol-2(3H)-one;
I-270 5-(2-(3-methoxy-4-(4-methylpiperazine-1-carbonyl)phenylamino)-5-methylpyrimidin-4-ylamino)benzo[d]oxazol-2(3H)-one;
I-271 5-(2-(3-(difluoromethyl)-4-methoxyphenylamino)-5-methylpyrimidin-4-ylamino)benzo[d]oxazol-2(3H)-one;
I-272 5-(2-(4-methoxyphenylamino)-5-methylpyrimidin-4-ylamino)benzo[d]oxazol-2(3H)-one;
I-273 5-(2-(3-(difluoromethyl)-5-methoxyphenylamino)-5-methylpyrimidin-4-ylamino)benzo[d]oxazol-2(3H)-one;
I-274 5-(2-(3-(fluoromethyl)-5-methoxyphenylamino)-5-methylpyrimidin-4-ylamino)benzo[d]oxazol-2(3H)-one;
I-275 N2-[4-(4,4-difluoropiperidinyl)-3-fluoro]phenyl-5-methyl-N4-(2-oxo-2,3-dihydro-1,3-benzoxazol-5-yl)-2,4-pyrimidinediamine;
I-276 N2-[4-(4,4-difluoropiperidinyl)-3-trifluoromethyl]phenyl-5-methyl-N4-(2-oxo-2,3-dihydro-1,3-benzoxazol-5-yl)-2,4-pyrimidinediamine;
I-277 N2-[3-chloro-4-(4,4-difluoropiperidinyl)]phenyl-5-methyl-N4-(2-oxo-2,3-dihydro-1,3-benzoxazol-5-yl)-2,4-pyrimidinediamine;
I-278 N2-[3-chloro-4-(4-ethylpiperazino)]phenyl-5-methyl-N4-(2-oxo-2,3-dihydro-1,3-benzoxazol-5-yl)-2,4-pyrimidinediamine;
I-279 N2-[4-(4,4-difluoropiperidinyl)]phenyl-5-methyl-N4-(2-oxo-2,3-dihydro-1,3-benzoxazol-5-yl)-2,4-pyrimidinediamine;
I-280 N2-(3,5-dimethoxy)phenyl-5-methyl-N4-(2-oxo-2,3-dihydro-1,3-benzoxazol-5-yl)-2,4-pyrimidinediamine;
I-281 N2-[3-fluoro-4-(4-methylpiperazino)]phenyl-5-methyl-N4-(2-oxo-2,3-dihydro-1,3-benzoxazol-5-yl)-2,4-pyrimidinediamine;
I-282 N2-[3,5-difluoro-4-(4-methylpiperazino)]phenyl-5-methyl-N4-(2-oxo-2,3-dihydro-1,3-benzoxazol-5-yl)-2,4-pyrimidinediamine;
I-283 N2-[4-chloro-3-(4-ethylpiperazino)]phenyl-5-methyl-N4-(2-oxo-2,3-dihydro-1,3-benzoxazol-5-yl)-2,4-pyrimidinediamine;
I-284 N2-[4-chloro-3-(3,4,5-trimethylpiperazino)]phenyl-5-methyl-N4-(2-oxo-2,3-dihydro-1,3-benzoxazol-5-yl)-2,4-pyrimidinediamine;
I-285 5-methyl-N4-(2-oxo-2,3-dihydro-1,3-benzoxazol-5-yl)-N2-[3-(4-propylpiperazino)-4-trifluoromethyl]phenyl-2,4-pyrimidinediamine;
I-286 5-methyl-N2-[3-(1,3-oxazol-5-yl)]phenyl-N4-(2-oxo-2,3-dihydro-1,3-benzoxazol-5-yl)-2,4-pyrimidinediamine;
I-287 N2-(3-bromo)phenyl-5-methyl-N4-(2-oxo-2,3-dihydro-1,3-benzoxazol-5-yl)-2,4-pyrimidinediamine;
I-288 N2-(4-bromo)phenyl-5-methyl-N4-(2-oxo-2,3-dihydro-1,3-benzoxazol-5-yl)-2,4-pyrimidinediamine;
I-289 5-methyl-N4-(2-oxo-2,3-dihydro-1,3-benzoxazol-5-yl)-N2-[3-(pyridin-4-yl)]phenyl-2,4-pyrimidinediamine;
I-290 5-methyl-N4-(2-oxo-2,3-dihydro-1,3-benzoxazol-5-yl)-N2-[3-(pyridin-3-yl)]phenyl-2,4-pyrimidinediamine;
I-291 5-methyl-N4-(2-oxo-2,3-dihydro-1,3-benzoxazol-5-yl)-N2-[4-(pyridin-3-yl)]phenyl-2,4-pyrimidinediamine;
I-292 N2-[4-methoxy-3-(2-methoxyethoxy)]phenyl-5-methyl-N4-(2-oxo-2,3-dihydro-1,3-benzoxazol-5-yl)-2,4-pyrimidinediamine;
I-293 N2-[3-(cyclopropylaminocarbonylmethoxy)-4-methoxy]phenyl-5-methyl-N4-(2-oxo-2,3-dihydro-1,3-benzoxazol-5-yl)-2,4-pyrimidinediamine;
I-294 N2-(3-cyano-4-fluoro)phenyl-5-methyl-N4-(2-oxo-2,3-dihydro-1,3-benzoxazol-5-yl)-2,4-pyrimidinediamine;
I-295 N2-[3-cyano-4-(1H-pyrrol-1-yl)]phenyl-5-methyl-N4-(2-oxo-2,3-dihydro-1,3-benzoxazol-5-yl)-2,4-pyrimidinediamine;
I-296 N2-(3-methoxy-5-trifluoromethyl)phenyl-5-methyl-N4-(2-oxo-2,3-dihydro-1,3-benzoxazol-5-yl)-2,4-pyrimidinediamine;
I-297 N2-(4-methoxy-3-trifluoromethyl)phenyl-5-methyl-N4-(2-oxo-2,3-dihydro-1,3-benzoxazol-5-yl)-2,4-pyrimidinediamine;
I-298 N2-{4-methoxy-3-[(pyridin-4-yl)methoxy]}phenyl-5-methyl-N4-(2-oxo-2,3-dihydro-1,3-benzoxazol-5-yl)-2,4-pyrimidinediamine;
I-299 N2-{4-methoxy-3-[(pyridin-3-yl)methoxy]}phenyl-5-methyl-N4-(2-oxo-2,3-dihydro-1,3-benzoxazol-5-yl)-2,4-pyrimidinediamine;
I-300 N2-{4-methoxy-3-[2-(dimethylamino)ethoxy]}phenyl-5-methyl-N4-(2-oxo-2,3-dihydro-1,3-benzoxazol-5-yl)-2,4-pyrimidinediamine;
I-301 N2-[3,5-bis(trifluoromethyl)]phenyl-5-methyl-N4-(2-oxo-2,3-dihydro-1,3-benzoxazol-5-yl)-2,4-pyrimidinediamine;
I-302 N2-(3,5-dimethyl)phenyl-5-methyl-N4-(2-oxo-2,3-dihydro-1,3-benzoxazol-5-yl)-2,4-pyrimidinediamine;
I-303 N2-(4-cyano-3-trifluoromethyl)phenyl-5-methyl-N4-(2-oxo-2,3-dihydro-1,3-benzoxazol-5-yl)-2,4-pyrimidinediamine;
I-304 N2-[3-(1-hydroxy-2,2,2-trifluoroethyl)]phenyl-5-methyl-N4-(2-oxo-2,3-dihydro-1,3-benzoxazol-5-yl)-2,4-pyrimidinediamine;
I-305 N2-(3-methoxycarbonylmethoxy)phenyl-5-methyl-N4-(2-oxo-2,3-dihydro-1,3-benzoxazol-5-yl)-2,4-pyrimidinediamine;
I-306 5-methyl-N2-(3-methylaminocarbonylmethoxy)phenyl-N4-(2-oxo-2,3-dihydro-1,3-benzoxazol-5-yl)-2,4-pyrimidinediamine;
I-307 N2-(4-aminocarbonylmethoxy)phenyl-5-methyl-N4-(2-oxo-2,3-dihydro-1,3-benzoxazol-5-yl)-2,4-pyrimidinediamine;
I-308 5-methyl-N4-(2-oxo-2,3-dihydro-1,3-benzoxazol-5-yl)-N2-(4-phenylcarbonylamino)phenyl-2,4-pyrimidinediamine;
I-309 N2-[4-(N-acetyl-N-methypamino]phenyl-5-methyl-N4-(2-oxo-2,3-dihydro-1,3-benzoxazol-5-yl)-2,4-pyrimidinediamine;
I-310 N2-[3-cyano-4-(pyrrolidin-1-yl)]phenyl-5-methyl-N4-(2-oxo-2,3-dihydro-1,3-benzoxazol-5-yl)-2,4-pyrimidinediamine;
I-311 N2-(4-difluoromethoxy)phenyl-5-methyl-N4-(2-oxo-2,3-dihydro-1,3-benzoxazol-5-yl)-2,4-pyrimidinediamine;
I-312 N2-(3-difluoromethoxy)phenyl-5-methyl-N4-(2-oxo-2,3-dihydro-1,3-benzoxazol-5-yl)-2,4-pyrimidinediamine;
I-313 N2-(4-difluoromethoxy-3-ethoxy)phenyl-5-methyl-N4-(2-oxo-2,3-dihydro-1,3-benzoxazol-5-yl)-2,4-pyrimidinediamine;
I-314 N2-(3-chloro-4-difluoromethoxy)phenyl-5-methyl-N4-(2-oxo-2,3-dihydro-1,3-benzoxazol-5-yl)-2,4-pyrimidinediamine;
I-315 N2-[3-(cyclopropylaminocarbonylmethoxy)]phenyl-5-methyl-N4-(2-oxo-2,3-dihydro-1,3-benzoxazol-5-yl)-2,4-pyrimidinediamine;
I-316 N2-[3-aminocarbonyl-4-(4-methylpiperazino)]phenyl-5-methyl-N4-(2-oxo-2,3-dihydro-1,3-benzoxazol-5-yl)-2,4-pyrimidinediamine;
I-317 N2-[4-(isopropoxycarbonylmethoxy)]phenyl-5-methyl-N4-(2-oxo-2,3-dihydro-1,3-benzoxazol-5-yl)-2,4-pyrimidinediamine;
I-318 N2-[4-(ethylaminocarbonylamino)]phenyl-5-methyl-N4-(2-oxo-2,3-dihydro-1,3-benzoxazol-5-yl)-2,4-pyrimidinediamine;
I-319 N2-[3-(aminocarbonylmethoxy)]phenyl-5-methyl-N4-(2-oxo-2,3-dihydro-1,3-benzoxazol-5-yl)-2,4-pyrimidinediamine;
I-320 5-methyl-N2-[3-(morpholinocarbonylmethoxy)]phenyl-N4-(2-oxo-2,3-dihydro-1,3-benzoxazol-5-yl)-2,4-pyrimidinediamine;
I-321 5-methyl-N2-[3-(4-methylpiperazin-1-yl)carbonyl]phenyl-N4-(2-oxo-2,3-dihydro-1,3-benzoxazol-5-yl)-2,4-pyrimidinediamine;
I-322 5-methyl-N2-[4-(4-methylpiperazin-1-yl)carbonyl]phenyl-N4-(2-oxo-2,3-dihydro-1,3-benzoxazol-5-yl)-2,4-pyrimidinediamine;
I-323 5-methyl-N2-[3-methylaminocarbonyl-4-(4-methylpiperazino)]phenyl-N4-(2-oxo-2,3-dihydro-1,3-benzoxazol-5-yl)-2,4-pyrimidinediamine;
I-324 N2-[4-(1-aminocarbonyl-1-methypethoxy]phenyl-5-methyl-N4-(2-oxo-2,3-dihydro-1,3-benzoxazol-5-yl)-2,4-pyrimidinediamine;
I-325 5-methyl-N2-(2-methyl-3-methylaminocarbonylmethoxy)phenyl-N4-(2-oxo-2,3-dihydro-1,3-benzoxazol-5-yl)-2,4-pyrimidinediamine;
I-326 N2-(3-dimethylaminocarbonylmethoxy)phenyl-5-methyl-N4-(2-oxo-2,3-dihydro-1,3-benzoxazol-5-yl)-2,4-pyrimidinediamine;
I-327 N2-(3-cyano-4-morpholino)phenyl-5-methyl-N4-(2-oxo-2,3-dihydro-1,3-benzoxazol-5-yl)-2,4-pyrimidinediamine;
I-328 N2-(3-methoxy-2-methyl)phenyl-5-methyl-N4-(2-oxo-2,3-dihydro-1,3-benzoxazol-5-yl)-2,4-pyrimidinediamine;
I-329 N2-[3-chloro-4-(pyridin-4-yl)]phenyl 5-methyl-N4-(2-oxo-2,3-dihydro-1,3-benzoxazol-5-yl)-2,4-pyrimidinediamine;
I-330 5-methyl-N4-(2-oxo-2,3-dihydro-1,3-benzoxazol-5-yl)-N2-[4-(pyridin-4-yl)-3-trifluoromethyl]phenyl-2,4-pyrimidinediamine;
I-331 N2-[3-hydroxymethyl-4-(4-methylpiperazino)]phenyl-5-methyl-N4-(2-oxo-2,3-dihydro-1,3-benzoxazol-5-yl)-2,4-pyrimidinediamine;
I-332 5-methyl-N4-(2-oxo-2,3-dihydro-1,3-benzoxazol-5-yl)-N2-(4-piperazino)phenyl-2,4-pyrimidinediamine;
I-333 N2-[4-(4-ethylaminocarbonyl)piperazino]phenyl-5-methyl-N4-(2-oxo-2,3-dihydro-1,3-benzoxazol-5-yl)-2,4-pyrimidinediamine;
I-334 N2-[4-(1-cyano-1-methypethoxy]phenyl-5-methyl-N4-(2-oxo-2,3-dihydro-1,3-benzoxazol-5-yl)-2,4-pyrimidinediamine;
I-335 N2-[3-(1-aminocarbonyl-1-methypethoxy]phenyl-5-methyl-N4-(2-oxo-2,3-dihydro-1,3-benzoxazol-5-yl)-2,4-pyrimidinediamine;
I-336 N2-(3-methoxy-4-methoxycarbonyl)phenyl-5-methyl-N4-(2-oxo-2,3-dihydro-1,3-benzoxazol-5-yl)-2,4-pyrimidinediamine;
I-337 N2-(3-methoxy)phenyl-5-methyl-N4-(2-oxo-2,3-dihydro-1,3-benzoxazol-5-yl)-2,4-pyrimidinediamine;
I-338 5-methyl-N2-(4-morpholino)phenyl-N4-(2-oxo-2,3-dihydro-1,3-benzoxazol-5-yl)-2,4-pyrimidinediamine;
I-339 N2-(3-cyano-4-thiomorpholino)phenyl-5-methyl-N4-(2-oxo-2,3-dihydro-1,3-benzoxazol-5-yl)-2,4-pyrimidinediamine;
I-340 N2-[3-methoxy-4-(4-methylpiperazino)]phenyl-5-methyl-N4-(2-oxo-2,3-dihydro-1,3-benzoxazol-5-yl)-2,4-pyrimidinediamine;
I-341 N2-[3-cyano-4-(4-methylpiperazino)]phenyl-5-methyl-N4-(2-oxo-2,3-dihydro-1,3-benzoxazol-5-yl)-2,4-pyrimidinediamine;
I-342 N2-[3-(1-cyano-1-methypethoxy]phenyl-5-methyl-N4-(2-oxo-2,3-dihydro-1,3-benzoxazol-5-yl)-2,4-pyrimidinediamine;
I-343 N2-[4-(4-acetyppiperazino]phenyl-5-methyl-N4-(2-oxo-2,3-dihydro-1,3-benzoxazol-5-yl)-2,4-pyrimidinediamine;
I-344 N2-[4-(4-ethoxycarbonyl)piperazino]phenyl-5-methyl-N4-(2-oxo-2,3-dihydro-1,3-benzoxazol-5-yl)-2,4-pyrimidinediamine;
I-345 N2-[3-(4-acetyppiperazino]phenyl-5-methyl-N4-(2-oxo-2,3-dihydro-1,3-benzoxazol-5-yl)-2,4-pyrimidinediamine;
I-346 N2-[3-(4-ethoxycarbonyl)piperazino]phenyl-5-methyl-N4-(2-oxo-2,3-dihydro-1,3-benzoxazol-5-yl)-2,4-pyrimidinediamine;
I-347 N2-(4-difluoromethoxy-3-fluoro)phenyl-5-methyl-N4-(2-oxo-2,3-dihydro-1,3-benzoxazol-5-yl)-2,4-pyrimidinediamine;
I-348 N2-(3,5-dichloro-4-difluoromethoxy)phenyl-5-methyl-N4-(2-oxo-2,3-dihydro-1,3-benzoxazol-5-yl)-2,4-pyrimidinediamine;
I-349 N2-(4-fluoro-3-methoxy)phenyl-5-methyl-N4-(2-oxo-2,3-dihydro-1,3-benzoxazol-5-yl)-2,4-pyrimidinediamine;
I-350 N2-(3-fluoro-4-methoxy)phenyl-5-methyl-N4-(2-oxo-2,3-dihydro-1,3-benzoxazol-5-yl)-2,4-pyrimidinediamine;
I-351 N2-(3-methoxy-4-methyl)phenyl-5-methyl-N4-(2-oxo-2,3-dihydro-1,3-benzoxazol-5-yl)-2,4-pyrimidinediamine;
I-352 N2-(3-fluoro-5-methoxy)phenyl-5-methyl-N4-(2-oxo-2,3-dihydro-1,3-benzoxazol-5-yl)-2,4-pyrimidinediamine;
I-353 N2-(3-difluoromethoxy-5-trifluoromethyl)phenyl-5-methyl-N4-(2-oxo-2,3-dihydro-1,3-benzoxazol-5-yl)-2,4-pyrimidinediamine;
I-354 N2-(3-methoxy-4-trifluoromethyl)phenyl-5-methyl-N4-(2-oxo-2,3-dihydro-1,3-benzoxazol-5-yl)-2,4-pyrimidinediamine;
I-355 N2-(3,5-di-tert-butyl)phenyl-5-methyl-N4-(2-oxo-2,3-dihydro-1,3-benzoxazol-5-yl)-2,4-pyrimidinediamine;
I-356 N4-{3-[bi s (1, 1-dimethylethoxy)]phosphinyloxymethyl-2-oxo-2,3-dihydro-1,3-benzoxazol-5-yl}-N2-(3-methoxy-5-trifluoromethyl)phenyl-5-methyl-2,4-pyrimidinediamine;
I-357 N2-(3-methoxy-5-trifluoromethyl)phenyl-5-methyl-N4-[3-(phosphonooxy)methyl-2-oxo-2,3-dihydro-1,3-benzoxazol-5-yl]-2,4-pyrimidinediamine;
I-358 N2-(3-methoxy-5-trifluoromethyl)phenyl-5-methyl-N4-[3-(phosphonooxy)methyl-2-oxo-2,3-dihydro-1,3-benzoxazol-5-yl]-2,4-pyrimidinediamine bis-sodium salt;
I-359 N2-(3,5-difluoro)phenyl-5-methyl-N4-(2-oxo-2,3-dihydro-1,3-benzoxazol-5-yl)-2,4-pyrimidinediamine;
I-360 N2-(3-fluoro-5-trifluoromethyl)phenyl-5-methyl-N4-(2-oxo-2,3-dihydro-1,3-benzoxazol-5-yl)-2,4-pyrimidinediamine;
I-361 N2-(4-fluoro-3-trifluoromethyl)phenyl-5-methyl-N4-(2-oxo-2,3-dihydro-1,3-benzoxazol-5-yl)-2,4-pyrimidinediamine;
I-362 N2-(4-fluoro-3-methyl)phenyl-5-methyl-N4-(2-oxo-2,3-dihydro-1,3-benzoxazol-5-yl)-2,4-pyrimidinediamine;
I-363 N2-(3-fluoro-4-methyl)phenyl-5-methyl-N4-(2-oxo-2,3-dihydro-1,3-benzoxazol-5-yl)-2,4-pyrimidinediamine;
I-364 N2-(3-chloro-4-methyl)phenyl-5-methyl-N4-(2-oxo-2,3-dihydro-1,3-benzoxazol-5-yl)-2,4-pyrimidinediamine;
I-365 N2-(3,4,5-trimethyl)phenyl-5-methyl-N4-(2-oxo-2,3-dihydro-1,3-benzoxazol-5-yl)-2,4-pyrimidinediamine;
I-366 N2-(3-chloro-4-trifluoromethoxy)phenyl-5-methyl-N4-(2-oxo-2,3-dihydro-1,3-benzoxazol-5-yl)-2,4-pyrimidinediamine;
I-367 N2-(4-trifluoromethylthio)phenyl-5-methyl-N4-(2-oxo-2,3-dihydro-1,3-benzoxazol-5-yl)-2,4-pyrimidinediamine;
I-368 N2-(3-fluoro)phenyl-5-methyl-N4-(2-oxo-2,3-dihydro-1,3-benzoxazol-5-yl)-2,4-pyrimidinediamine;
I-369 N2-(3,5-dimethyl-4-methoxy)phenyl-5-methyl-N4-(2-oxo-2,3-dihydro-1,3-benzoxazol-5-yl)-2,4-pyrimidinediamine;
I-370 N2-(3-carboxamide-5-trifluoromethyl)phenyl-5-methyl-N4-(2-oxo-2,3-dihydro-1,3-benzoxazol-5-yl)-2,4-pyrimidinediamine;
I-371 N2-(3,5-diisopropyl-4-methoxy)phenyl-5-methyl-N4-(2-oxo-2,3-dihydro-1,3-benzoxazol-5-yl)-2,4-pyrimidinediamine;
I-372 N2-(3-isopropoxy-5-trifluoromethyl)phenyl-5-methyl-N4-(2-oxo-2,3-dihydro-1,3-benzoxazol-5-yl)-2,4-pyrimidinediamine;
I-373 N2-(3-cyano-4-methoxy)phenyl-5-methyl-N4-(2-oxo-2,3-dihydro-1,3-benzoxazol-5-yl)-2,4-pyrimidinediamine;
I-374 N2-(3,5-dimethyl-4-fluoro)phenyl-5-methyl-N4-(2-oxo-2,3-dihydro-1,3-benzoxazol-5-yl)-2,4-pyrimidinediamine;
I-375 N2-(4-fluoro-3-trifluoromethoxy)phenyl-5-methyl-N4-(2-oxo-2,3-dihydro-1,3-benzoxazol-5-yl)-2,4-pyrimidinediamine;
I-376 N2-(3-fluoro-4-trifluoromethoxy)phenyl-5-methyl-N4-(2-oxo-2,3-dihydro-1,3-benzoxazol-5-yl)-2,4-pyrimidinediamine;
I-377 N2-(4-chloro-3-trifluoromethoxy)phenyl-5-methyl-N4-(2-oxo-2,3-dihydro-1,3-benzoxazol-5-yl)-2,4-pyrimidinediamine;
I-378 N2-(3-chloro-5-trifluoromethoxy)phenyl-5-methyl-N4-(2-oxo-2,3-dihydro-1,3-benzoxazol-5-yl)-2,4-pyrimidinediamine;
I-379 5-methyl-N2-(3-methyl-5-trifluoromethoxy)phenyl-N4-(2-oxo-2,3-dihydro-1,3-benzoxazol-5-yl)-2,4-pyrimidinediamine;
I-380 N2-(4-cyano-3-methoxy)phenyl-5-methyl-N4-(2-oxo-2,3-dihydro-1,3-benzoxazol-5-yl)-2,4-pyrimidinediamine;
I-381 N2-(3,5-difluoro-4-methoxy)phenyl-5-methyl-N4-(2-oxo-2,3-dihydro-1,3-benzoxazol-5-yl)-2,4-pyrimidinediamine;
I-382 5-methyl-N2-(4-morpholinomethyl)phenyl-N4-(2-oxo-2,3-dihydro-1,3-benzoxazol-5-yl)-2,4-pyrimidinediamine;
I-383 N2-(4-chloro-3-cyano-5-ethyl)phenyl-5-methyl-N4-(2-oxo-2,3-dihydro-1,3-benzoxazol-5-yl)-2,4-pyrimidinediamine;
I-384 N2-[3-(2-methoxy)ethoxy-5-trifluoromethyl]phenyl-5-methyl-N4-(2-oxo-2,3-dihydro-1,3-benzoxazol-5-yl)-2,4-pyrimidinediamine;
I-385 N2-(4-difluoromethoxy-3,5-dimethyl)phenyl-5-methyl-N4-(2-oxo-2,3-dihydro-1,3-benzoxazol-5-yl)-2,4-pyrimidinediamine;
I-386 N2-[3-(1-aminocarbonyl-1-methypethoxy-4-fluoro]phenyl-5-methyl-N4-(2-oxo-2,3-dihydro-1,3-benzoxazol-5-yl)-2,4-pyrimidinediamine;
I-387 N2-(4-difluoromethoxy-3-methyl)phenyl-5-methyl-N4-(2-oxo-2,3-dihydro-1,3-benzoxazol-5-yl)-2,4-pyrimidinediamine;
I-388 N2-(3,5-difluoro-4-difluoromethoxy)phenyl-5-methyl-N4-(2-oxo-2,3-dihydro-1,3-benzoxazol-5-yl)-2,4-pyrimidinediamine;
I-389 N2-[4-(1-aminocarbonyl-1-methypethoxy-3,5-dimethyl]phenyl-5-methyl-N4-(2-oxo-2,3-dihydro-1,3-benzoxazol-5-yl)-2,4-pyrimidinediamine;
I-390 N2-(3-difluoromethoxy-4-methyl)phenyl-5-methyl-N4-(2-oxo-2,3-dihydro-1,3-benzoxazol-5-yl)-2,4-pyrimidinediamine;
I-391 N2-[4-(1-aminocarbonyl-1-methypethoxy-3-methyl]phenyl-5-methyl-N4-(2-oxo-2,3-dihydro-1,3-benzoxazol-5-yl)-2,4-pyrimidinediamine;
I-392 N2-[3-(1-aminocarbonyl-1-methypethoxy-4-methyl]phenyl-5-methyl-N4-(2-oxo-2,3-dihydro-1,3-benzoxazol-5-yl)-2,4-pyrimidinediamine;
I-393 N2-(3-methoxy-5-trifluoromethyl)phenyl-5-methyl-N4-(2-oxo-2,3-dihydro-1,3-benzoxazol-5-yl)-2,4-pyrimidinediamine besylate salt;
I-394 N2-(4-chloro-3,5-dimethyl)phenyl-5-methyl-N4-(2-oxo-2,3-dihydro-1,3-benzoxazol-5-yl)-2,4-pyrimidinediamine;
I-395 N2-[4-(1-aminocarbonyl-1-methypethoxy-3,5-difluoro]phenyl-5-methyl-N4-(2-oxo-2,3-dihydro-1,3-benzoxazol-5-yl)-2,4-pyrimidinediamine;
I-396 N2-[3-(1-methoxy-2,2,2-trifluoroethyl)]phenyl-5-methyl-N4-(2-oxo-2,3-dihydro-1,3-benzoxazol-5-yl)-2,4-pyrimidinediamine;
I-397 N2-[3-(1-cyano-1-methypethoxy-4-methyl]phenyl-5-methyl-N4-(2-oxo-2,3-dihydro-1,3-benzoxazol-5-yl)-2,4-pyrimidinediamine;
I-398 N2-(3,4-difluoro)phenyl-5-methyl-N4-(2-oxo-2,3-dihydro-1,3-benzoxazol-5-yl)-2,4-pyrimidinediamine;
I-399 N2-(3-chloro-4-fluoro)phenyl-5-methyl-N4-(2-oxo-2,3-dihydro-1,3-benzoxazol-5-yl)-2,4-pyrimidinediamine;
I-400 N2-(4-chloro-3-fluoro)phenyl-5-methyl-N4-(2-oxo-2,3-dihydro-1,3-benzoxazol-5-yl)-2,4-pyrimidinediamine;
I-401 N2-(3-difluoromethoxy-5-fluoro)phenyl-5-methyl-N4-(2-oxo-2,3-dihydro-1,3-benzoxazol-5-yl)-2,4-pyrimidinediamine;
I-402 N2-[3-(1-aminocarbonyl-1-methypethoxy-5-fluoro]phenyl-5-methyl-N4-(2-oxo-2,3-dihydro-1,3-benzoxazol-5-yl)-2,4-pyrimidinediamine;
I-403 5-(5-Methyl-2-m-tolylamino-pyrimidin-4-ylamino)-3H-benzooxazol-2-one;
I-404 5-{2-[4-(3-Dimethylamino-propoxy)-3-trifluoromethyl-phenylamino]-5-methyl-pyrimidin-4-ylamino}-3H-benzooxazol-2-one;
I-405 N4-{3-[bis(1,1-dimethylethoxy)]phosphinyloxymethyl-2-oxo-2,3-dihydro-1,3-benzoxazol-5-yl}-N2-(3,4,5-trimethyl)phenyl-5-methyl-2,4-pyrimidinediamine;
I-406 5-methyl-N4-[3-(phosphonooxy)methyl-2-oxo-2,3-dihydro-1,3-benzoxazol-5-yl]-N2-(3,4,5-trimethyl)phenyl-2,4-pyrimidinediamine;
I-407 5-methyl-N4-[3-(phosphonooxy)methyl-2-oxo-2,3-dihydro-1,3-benzoxazol-5-yl]-N2-(3,4,5-trimethyl)phenyl-2,4-pyrimidinediamine bis-sodium salt;
I-408 5-methyl-N4-(2-oxo-2,3-dihydro-1,3-benzoxazol-5-yl)-N2-(3,4,5-trifluoro)phenyl-2,4-pyrimidinediamine;
I-409 N2-(3-methoxy-5-trifluoromethyl)phenyl-5-methyl-N4-(2-oxo-2,3-dihydro-1,3-benzoxazol-5-yl)-2,4-pyrimidinediamine tosylate salt;
I-410 N2-(3-methoxy-5-trifluoromethyl)phenyl-5-methyl-N4-(2-oxo-2,3-dihydro-1,3-benzoxazol-5-yl)-2,4-pyrimidinediamine mesylate salt;
I-411 N2-(3-methoxy-5-trifluoromethyl)phenyl-5-methyl-N4-(2-oxo-2,3-dihydro-1,3-benzoxazol-5-yl)-2,4-pyrimidinediamine sulfate salt;
I-412 N2-(3-methoxy-5-trifluoromethyl)phenyl-5-methyl-N4-(2-oxo-2,3-dihydro-1,3-benzoxazol-5-yl)-2,4-pyrimidinediamine hydrogen chloride salt;
I-413 N2-(3-methoxy-5-trifluoromethyl)phenyl-5-methyl-N4-(2-oxo-2,3-dihydro-1,3-benzoxazol-5-yl)-2,4-pyrimidinediamine sodium salt;
I-414 N2-(3-methoxy-5-trifluoromethyl)phenyl-5-methyl-N4-(2-oxo-2,3-dihydro-1,3-benzoxazol-5-yl)-2,4-pyrimidinediamine choline salt;
I-415 N2-(3,5-difluoro-4-trifluoromethyl)phenyl-5-methyl-N4-(2-oxo-2,3-dihydro-1,3-benzoxazol-5-yl)-2,4-pyrimidinediamine;
I-416 N2-[3-(1-cyano-1-methypethoxy-5-fluoro]phenyl-5-methyl-N4-(2-oxo-2,3-dihydro-1,3-benzoxazol-5-yl)-2,4-pyrimidinediamine;
I-417 N2-[3-(1-cyano-1-methypethoxy-4-fluoro]phenyl-5-methyl-N4-(2-oxo-2,3-dihydro-1,3-benzoxazol-5-yl)-2,4-pyrimidinediamine;
I-418 N2-(4-chloro-3-difluoromethoxy)phenyl-5-methyl-N4-(2-oxo-2,3-dihydro-1,3-benzoxazol-5-yl)-2,4-pyrimidinediamine;
I-419 5-(2-(4-isopropylphenylamino)-5-methylpyrimidin-4-ylamino)benzo[d]oxazol-2(3H)-one;
I-420 5-(2-(4-tert-butylphenylamino)-5-methylpyrimidin-4-ylamino)benzo[d]oxazol-2(3H)-one;
I-421 5-(2-(p-toluidino)-5-methylpyrimidin-4-ylamino)benzo[d]oxazol-2(3H)-one;
I-422 5-(2-(3-(isopropoxymethyl)-4-methoxyphenylamino)-5-methylpyrimidin-4-ylamino)benzo[d]oxazol-2(3H)-one;
I-423 5-(2-(3-(1-hydroxyethyl)-5-methoxyphenylamino)-5-methylpyrimidin-4-ylamino)benzo[d]oxazol-2(3H)-one;
I-424 5-[2-(3-Chloro-4-hydroxy-phenylamino)-5-methyl-pyrimidin-4-ylamino]-3H-benzooxazol-2-one;
I-425 5-[2-(4-Hydroxy-phenylamino)-5-methyl-pyrimidin-4-ylamino]-3H-benzooxazol-2-one;
I-426 5-{2-[4-(2-Dimethylamino-ethoxy)-phenylamino]-5-methyl-pyrimidin-4-ylamino}-3H-benzooxazol-2-one;
I-427 5-(2-(3-methoxy-5-(trifluoromethyl)phenylamino)-5-methylpyrimidin-4-ylamino)-7-methylbenzo[d]oxazol-2(3H)-one;
I-428 5-(2-(3-methoxy-5-methylphenylamino)-5-methylpyrimidin-4-ylamino)-7-methylbenzo[d]oxazol-2(3H)-one;
I-429 5-(2-(4-methoxy-3-methylphenylamino)-5-methylpyrimidin-4-ylamino)-7-methylbenzo[d]oxazol-2(3H)-one;
I-430 7-fluoro-5-(2-(3-methoxy-5-methylphenylamino)-5-methylpyrimidin-4-ylamino)benzo[d]oxazol-2(3H)-one;
I-431 7-fluoro-5-(2-(4-methoxy-3-methylphenylamino)-5-methylpyrimidin-4-ylamino)benzo[d]oxazol-2(3H)-one;
I-432 5-(2-(4-fluoro-3-methoxy-5-methylphenylamino)-5-methylpyrimidin-4-ylamino)benzo[d]oxazol-2(3H)-one;
I-433 5-(2-(4-(difluoromethoxy)-3-(fluoromethyl)phenylamino)-5-methylpyrimidin-4-ylamino)benzo[d]oxazol-2(3H)-one formate salt;
I-434 N2-(4-cyano-3-difluoromethoxy)phenyl-5-methyl-N4-(2-oxo-2,3-dihydro-1,3-benzoxazol-5-yl)-2,4-pyrimidinediamine;
I-435 N2-(3-difluoromethoxy-4-fluoro)phenyl-5-methyl-N4-(2-oxo-2,3-dihydro-1,3-benzoxazol-5-yl)-2,4-pyrimidinediamine;
I-436 5-methyl-N4-(2-oxo-2,3-dihydro-1,3-benzoxazol-5-yl)-N2-(3,4,5-trimethyl)phenyl-2,4-pyrimidinediamine sodium salt;
I-437 N2-(3,5-dimethyl-4-fluoro)phenyl-5-methyl-N4-(2-oxo-2,3-dihydro-1,3-benzoxazol-5-yl)-2,4-pyrimidinediamine sodium salt;
I-438 5-(2-(3-(difluoromethyl)-4-methylphenylamino)-5-methylpyrimidin-4-ylamino)benzo[d]oxazol-2(3H)-one;
I-439 5-(2-(3-(fluoromethyl)-4-methylphenylamino)-5-methylpyrimidin-4-ylamino)benzo[d]oxazol-2(3H)-one;
I-440 5-(2-(3-(difluoromethyl)-5-methoxyphenylamino)-5-methylpyrimidin-4-ylamino)benzo[d]oxazol-2(3H)-one formate salt;
I-441 5-(2-(4-d₃-methoxy-3-(trifluoromethyl)phenylamino)-5-methylpyrimidin-4-ylamino)benzo[d]oxazol-2(3H)-one;
I-442 5-(2-(4-(difluoromethoxy)-3-(difluoromethyl)phenylamino)-5-methylpyrimidin-4-ylamino)benzo[d]oxazol-2(3H)-one formate salt;
I-443 5-(5-methyl-2-(4-methyl-3-(pyridin-4-yl)phenylamino)pyrimidin-4-ylamino)benzo[d]oxazol-2(3H)-one;
I-444 5-(5-methyl-2-(4-methyl-3-(pyridin-3-yl)phenylamino)pyrimidin-4-ylamino)benzo[d]oxazol-2(3H)-one;
I-445 5-(2-(3-acetyl-5-(trifluoromethyl)phenylamino)-5-methylpyrimidin-4-ylamino)benzo[d]oxazol-2(3H)-one;
I-446 5-(2-(3-(1-hydroxyethyl)-5-(trifluoromethyl)phenylamino)-5-methylpyrimidin-4-ylamino)benzo[d]oxazol-2(3H)-one;
I-447 5-[2-(4-d₃-Methoxy-phenylamino)-5-methyl-pyrimidin-4-ylamino]-3H-benzooxazol-2-one;
I-448 5-[2-(3-Chloro-4-d₃-methoxy-phenylamino)-5-methyl-pyrimidin-4-ylamino]-3H-benzooxazol-2-one;
I-449 5-{2-[4-(2-Diethylamino-ethoxy)-phenylamino]-5-methyl-pyrimidin-4-ylamino}-3H-benzooxazol-2-one;
I-450 N4-{3-[bis(1, 1-dimethylethoxy)]phosphinyloxymethyl-2-oxo-2,3-dihydro-1,3-benzoxazol-5-yl}-N2-(3,5-dimethyl-4-fluoro)phenyl-5-methyl-2,4-pyrimidinediamine;
I-451 N2-(3,5-dimethyl-4-fluoro)phenyl-5-methyl-N4-[3-(phosphonooxy)methyl-2-oxo-2,3-dihydro-1,3-benzoxazol-5-yl]-2,4-pyrimidinediamine bis-sodium salt;
I-452 5-(2-(3,4-dimethoxy-5-methylphenylamino)-5-methylpyrimidin-4-ylamino)benzo[d]oxazol-2(3H)-one;
I-453 5-(2-(3,4-dimethoxy-5-methylphenylamino)-5-methylpyrimidin-4-ylamino)-7-methylbenzo[d]oxazol-2(3H)-one;
I-454 5-(2-(3,4-dimethoxy-5-methylphenylamino)-5-methylpyrimidin-4-ylamino)-7-fluorobenzo[d]oxazol-2(3H)-one;
I-455 5-{2-[3-Chloro-4-(2-diethylamino-ethoxy)-phenylamino]-5-methyl-pyrimidin-4-ylamino}-3H-benzooxazol-2-one;
I-456 5-[2-(2,4-Difluoro-phenylamino)-5-methyl-pyrimidin-4-ylamino]-3H-benzooxazol-2-one;
I-457 5-(5-methyl-2-(3-(1-(methylamino)ethyl)-5-(trifluoromethyl)phenylamino)pyrimidin-4-ylamino)benzo[d]oxazol-2(3H)-one;
I-458 5-(2-(3-chloro-4,5-dimethoxyphenylamino)-5-methylpyrimidin-4-ylamino)benzo[d]oxazol-2(3H)-one;
I-459 5-(2-(3,5-dimethyl-4-(2-morpholinoethoxy)phenylamino)-5-methylpyrimidin-4-ylamino)benzo[d]oxazol-2(3H)-one;
I-460 5-(5-methyl-2-(3-(1-(methylamino)butyl)-5-(trifluoromethyl)phenylamino)pyrimidin-4-ylamino)benzo[d]oxazol-2(3H)-one;
I-461 5-(2-(3-(1-(cyclopropylamino)ethyl)-5-(trifluoromethyl)phenylamino)-5-methylpyrimidin-4-ylamino)benzo[d]oxazol-2(3H)-one;
I-462 5-(2-(3-(1-(ethylamino)ethyl)-5-(trifluoromethyl)phenylamino)-5-methylpyrimidin-4-ylamino)benzo[d]oxazol-2(3H)-one;
I-463 5-(5-methyl-2-(3-(1-(pyrrolidin-1-yl)ethyl)-5-(trifluoromethyl)phenylamino)pyrimidin-4-ylamino)benzo[d]oxazol-2(3H)-one;
I-464 5-(2-(3-(1-(azetidin-1-yl)ethyl)-5-(trifluoromethyl)phenylamino)-5-methylpyrimidin-4-ylamino)benzo[d]oxazol-2(3H)-one;
I-465 5-(2-(3-(1-(cyclobutylamino)ethyl)-5-(trifluoromethyl)phenylamino)-5-methylpyrimidin-4-ylamino)benzo[d]oxazol-2(3H)-one;
I-466 5-[2-(2,5-Difluoro-phenylamino)-5-methyl-pyrimidin-4-ylamino]-3H-benzooxazol-2-one;
I-467 5-[2-(2,3-Difluoro-phenylamino)-5-methyl-pyrimidin-4-ylamino]-3H-benzooxazol-2-one;
I-468 5-[2-(2-Fluoro-phenylamino)-5-methyl-pyrimidin-4-ylamino]-3H-benzooxazol-2-one;
I-469 N-Cyclobutyl-3-[5-methyl-4-(2-oxo-2,3-dihydro-benzooxazol-5-ylamino)-pyrimidin-2-ylamino]-5-trifluoromethyl-benzamide;
I-470 5-[2-(4-Fluoro-phenylamino)-5-methyl-pyrimidin-4-ylamino]-3H-benzooxazol-2-one;
I-471 5-(2-(4-fluoro-3-(pyridin-4-yl)phenylamino)-5-methylpyrimidin-4-ylamino)benzo[d]oxazol-2(3H)-one;
I-472 5-(2-(4-fluoro-3-(pyridin-3-yl)phenylamino)-5-methylpyrimidin-4-ylamino)benzo Ploxazol-2(3H)- one;
I-473 5-(2-(3-(1-(isopropylamino)ethyl)-5-(trifluoromethyl)phenylamino)-5-methylpyrimidin-4-ylamino)benzo[d]oxazol-2(3H)-one;
I-474 5-(2-(3,5-dimethylphenylamino)-5-methylpyrimidin-4-ylamino)-7-methylbenzo[d]oxazol-2(3H)-one;
I-475 7-methyl-5-(5-methyl-2-(3,4,5-trimethylphenylamino)pyrimidin-4-ylamino)benzo[d]oxazol-2(3H)-one;
I-476 5-(2-(4-fluoro-3,5-dimethylphenylamino)-5-methylpyrimidin-4-ylamino)-7-methylbenzo[d]oxazol-2(3H)-one;
I-477 5-[5-Methyl-2-(2,3,4,5-tetrafluoro-phenylamino)-pyrimidin-4-ylamino]-3H-benzooxazol-2-one;
I-478 N2-(3-cyano-5-difluoromethoxy)phenyl-5-methyl-N4-(2-oxo-2,3-dihydro-1,3-benzoxazol-5-yl)-2,4-pyrimidinediamine;
I-479 5-methyl-N2-(3-methyl-5-trifluoromethyl)phenyl-N4-(2-oxo-2,3-dihydro-1,3-benzoxazol-5-yl)-2,4-pyrimidinediamine;
I-480 5-[5-Methyl-2-(2,3,5-trifluoro-phenylamino)-pyrimidin-4-ylamino]-3H-benzooxazol-2-one;
I-481 5-[5-Methyl-2-(2, 4,5-trifluoro-phenylamino)-pyrimidin-4-ylamino]-3H-benzooxazol-2-one;
I-482 5-(5-methyl-2-(3-methyl-4-(pyridin-4-yl)phenylamino)pyrimidin-4-ylamino)benzo[d]oxazol-2(3H)-one formate salt;
I-483 5-(5-methyl-2-(3-methyl-4-(pyridin-3-yl)phenylamino)pyrimidin-4-ylamino)benzo[d]oxazol-2(3H)- one formate salt;
I-484 5-(2-(3-fluoro-4-(pyridin-4-yl)phenylamino)-5-methylpyrimidin-4-ylamino)benzo[d]oxazol-2(3H)-one;
I-485 N2-(3,4-dimethoxy-5-trifluoromethyl)phenyl-5-methyl-N4-(2-oxo-2,3-dihydro-1,3-benzoxazol-5-yl)-2,4-pyrimidinediamine;
I-486 5-(2-(4-methoxy-3-(pyridin-4-yl)phenylamino)-5-methylpyrimidin-4-ylamino)benzo[d]oxazol-2(3H)-one formate salt;
I-487 5-(2-(4-methoxy-3-(pyridin-3-yl)phenylamino)-5-methylpyrimidin-4-ylamino)benzo[d]oxazol-2(3H)-one formate salt;
I-488 5-(2-(3,5-dimethylphenylamino)-5-methylpyrimidin-4-ylamino)-7-fluorobenzo[d]oxazol-2(3H)-one;
I-489 7-fluoro-5-(5-methyl-2-(3,4,5-trimethylphenylamino)pyrimidin-4-ylamino)benzo[d]oxazol-2(3H)-one;
I-490 7-fluoro-5-(2-(4-fluoro-3,5-dimethylphenylamino)-5-methylpyrimidin-4-ylamino)benzo[d]oxazol-2(3H)-one;
I-491 5-(2-(4-fluoro-3-methoxy-5-methylphenylamino)-5-methylpyrimidin-4-ylamino)-7-methylbenzo[d]oxazol-2(3H)-one;
I-492 7-fluoro-5-(2-(4-fluoro-3-methoxy-5-methylphenylamino)-5-methylpyrimidin-4-ylamino)benzo[d]oxazol-2(3H)-one;
I-493 N2-(3,4-dimethyl-2-fluoro)phenyl-5-methyl-N4-(2-oxo-2,3-dihydro-1,3-benzoxazol-5-yl)-2,4-pyrimidinediamine;
I-494 5-(2-(3-methoxy-4-(pyridin-4-yl)phenylamino)-5-methylpyrimidin-4-ylamino)benzo[d]oxazol-2(3H)-one formate salt;
I-495 N2-(3-chloro-5-difluoromethoxy)phenyl-5-methyl-N4-(2-oxo-2,3-dihydro-1,3-benzoxazol-5-yl)-2,4-pyrimidinediamine;
I-496 5-[2-(3-Chloro-4-methoxy-phenylamino)-5-methyl-pyrimidin-4-ylamino]-3H-benzooxazol-2-one;
I-497 5-[2-(3-Chloro-5-trifluoromethyl-phenylamino)-5-methyl-pyrimidin-4-ylamino]-3H-benzooxazol-2-one;
I-498 5-[2-(2-Methoxy-5-trifluoromethyl-phenylamino)-5-methyl-pyrimidin-4-ylamino]-3H-benzooxazol-2-one;
I-499 5-(2-(o-toluidino)-5-methylpyrimidin-4-ylamino)benzo[d]oxazol-2(3H)-one;
I-500 5-(2-(2,3-dimethylphenylamino)-5-methylpyrimidin-4-ylamino)benzo[d]oxazol-2(3H)-one;
I-501 5-(2-(2,5-dimethylphenylamino)-5-methylpyrimidin-4-ylamino)benzo[d]oxazol-2(3H)-one;
I-502 5-(2-(2-ethylphenylamino)-5-methylpyrimidin-4-ylamino)benzo[d]oxazol-2(3H)-one;
I-503 5-(2-(3-ethylphenylamino)-5-methylpyrimidin-4-ylamino)benzo[d]oxazol-2(3H)-one;
I-504 5-(2-(4-ethylphenylamino)-5-methylpyrimidin-4-ylamino)benzo[a]oxazol-2(3H)-one;
I-505 5-(2-(3-fluoro-4-(pyridin-3-yl)phenylamino)-5-methylpyrimidin-4-ylamino)benzo[d]oxazol-2(3H)-one trifluoroacetic acid salt;
I-506 5-(2-(3-methoxy-4-(pyridin-3-yl)phenylamino)-5-methylpyrimidin-4-ylamino)benzo[d]oxazol-2(3H)-one formate salt;
I-507 5-(2-(2,4-difluoro-3-methoxyphenylamino)-5-methylpyrimidin-4-ylamino)-7-methylbenzo[d]oxazol-2(3H)-one;
I-508 5-(2-(2,4-difluoro-3-methoxyphenylamino)-5-methylpyrimidin-4-ylamino)-7-fluorobenzo[d]oxazol-2(3H)-one;
I-509 5-(2-(4-(6-chloropyridin-3-yl)phenylamino)-5-methylpyrimidin-4-ylamino)benzo[d]oxazol-2(3H)-one trifluoroacetate salt;
I-510 5-(2-(4-(6-(3-(dimethylamino)propoxy)pyridin-3-yl)phenylamino)-5-methylpyrimidin-4-ylamino)benzo[d]oxazol-2(3H)-one trifluoroacetate salt;
I-511 5-(2-(4-(6-(3-(dimethylamino)propoxy)pyridin-3-yl)phenylamino)-5-methylpyrimidin-4-ylamino)benzo[d]oxazol-2(3H)-one trifluoroacetate salt;
I-512 5-(5-methyl-2-(4-(6-morpholinopyridin-3-yl)phenylamino)pyrimidin-4-ylamino)benzo[d]oxazol-2(3H)-one trifluoroacetate salt;
I-513 5-(2-(2-fluoro-3-methylphenylamino)-5-methylpyrimidin-4-ylamino)benzo[d]oxazol-2(3H)-one;
I-514 5-(2-(2-fluoro-4-methylphenylamino)-5-methylpyrimidin-4-ylamino)benzo[d]oxazol-2(3H)-one;
I-515 5-(2-(2-fluoro-5-methylphenylamino)-5-methylpyrimidin-4-ylamino)benzo[d]oxazol-2(3H)-one;
I-516 N2-(3-difluoromethoxy-5-methyl)phenyl-5-methyl-N4-(2-oxo-2,3-dihydro-1,3-benzoxazol-5-yl)-2,4-pyrimidinediamine;
I-517 5-methyl-N4-[3-(phosphonooxy)methyl-2-oxo-2,3-dihydro-1,3-benzoxazol-5-yl]-N2-(3,4,5-trimethyl)phenyl-2,4-pyrimidinediamine calcium salt;
I-518 5-[5-Methyl-2-(2-methyl-3-trifluoromethyl-phenylamino)-pyrimidin-4-ylamino]-3H-benzooxazol-2-one;
I-519 5-(2-(5-acetyl-2-fluorophenylamino)-5-methylpyrimidin-4-ylamino)benzo[d]oxazol-2(3H)-one;
I-520 5-(2-(2-chlorophenylamino)-5-methylpyrimidin-4-ylamino)benzo[d]oxazol-2(3H)-one;
I-521 5-(2-(2-chloro-5-methylphenylamino)-5-methylpyrimidin-4-ylamino)benzo[d]oxazol-2(3H)-one;
I-522 N4-(7-chloro-2-oxo-2,3-dihydro-1,3-benzoxazol-5-yl)-5-methyl-N2-(3,4,5-trimethyl)phenyl-2,4-pyrimidinediamine;
I-523 5-(2-(2-fluoro-5-(1-hydroxyethyl)phenylamino)-5-methylpyrimidin-4-ylamino)benzo[d]oxazol-2(3H)-one;
I-524 N4-{3-[bis(1,1-dimethylethoxy)]phosphinyloxymethyl-7-chloro-2-oxo-2,3-dihydro-1,3-benzoxazol-5-yl}-N2-(3,4,5-trimethyl)phenyl-5-methyl-2,4-pyrimidinediamine;
I-525 N4-[7-chloro-3-(phosphonooxy)methyl-2-oxo-2,3-dihydro-1,3-benzoxazol-5-yl]-5-methyl-N2-(3,4,5-trimethyl)phenyl-2,4-pyrimidinediamine;
I-526 5-methyl-N4-[3-(phosphonooxy)methyl-2-oxo-2,3-dihydro-1,3-benzoxazol-5-yl]-N2-(3,4,5-trimethyl)phenyl-2,4-pyrimidinediamine magnesium salt;
I-527 5-[2-(4-Iodo-3,5-dimethyl-phenylamino)-5-methyl-pyrimidin-4-ylamino]-3H-benzooxazol-2-one;
I-528 N4-[7-chloro-3-(phosphonooxy)methyl-2-oxo-2,3-dihydro-1,3-benzoxazol-5-yl]-5-methyl-N2-(3,4,5-trimethyl)phenyl-2,4-pyrimidinediamine bis-sodium salt;
I-529 5-(2-(3,5-dimethoxy-4-methylphenylamino)-5-methylpyrimidin-4-ylamino)benzo[d]oxazol-2(3H)-one;
I-530 5-(2-(2-fluoro-4,5-dimethylphenylamino)-5-methylpyrimidin-4-ylamino)benzo[d]oxazol-2(3H)-one;
I-531 5-methyl-N4-[3-(phosphonooxy)methyl-2-oxo-2,3-dihydro-1,3-benzoxazol-5-yl]-N2-(3,4,5-trimethyl)phenyl-2,4-pyrimidinediamine bis-choline salt;
I-532 5-(2-(2-fluoro-4-methyl-3-(trifluoromethyl)phenylamino)-5-methylpyrimidin-4-ylamino)benzo[d]oxazol-2(3H)-one;
I-533 5-(2-(2-fluoro-5-methoxyphenylamino)-5-methylpyrimidin-4-ylamino)benzo[d]oxazol-2(3H)-one;
I-534 5-(2-(2-fluoro-3,4,5-trimethylphenylamino)-5-methylpyrimidin-4-ylamino)benzo[d]oxazol-2(3H)-one;
I-535 5-(2-(3-methoxy-4,5-dimethylphenylamino)-5-methylpyrimidin-4-ylamino)benzo[d]oxazol-2(3H)-one;
I-536 Sodium (5-(2-(3-methoxy-5-methylphenylamino)-5-methylpyrimidin-4-ylamino)-7-methyl-2-oxobenzo[d]oxazol-3(2H)-yl)methyl phosphate;
I-537 N2-(3,4-dimethyl-5-fluoro)phenyl-5-methyl-N4-(2-oxo-2,3-dihydro-1,3-benzoxazol-5-yl)-2,4-pyrimidinediamine;
I-538 Sodium (5-(2-(4-fluoro-3-methoxy-5-methylphenylamino)-5-methylpyrimidin-4-ylamino)-2-oxobenzo[d]oxazol-3(2H)-yl)methyl phosphate;
II-1 N4-(benzoxazol-2 (3H)-on-5-yl)-N2-(6-dimethylaminopyridin-3-yl)-5-methylpyrimidine-2,4-diamine;
II-2 N4-(benzo[d]oxazol-2 (3H)-on-5-yl)-N2-(6-((1 S,4R)-5-methyl-2,5-diazabicyclo[2.2.1]heptan-2-yl)-pyridin-3-yl)-5-methylpyrimidine-2,4-diamine;
II-3 N4-(benzoxazol-2 (3H)-on-5-yl)-N2-(6-(4-methyl-1,4-diazepan-1-yl)pyridin-3-yl)-5-methylpyrimidine-2,4-diamine;
II-4 N4-(3-n-propylbenzoxazol-2 (3H)-on-5-yl)-N2-(6-(4-methylpiperazin-1-yl)pyridin-3-yl)-5-methylpyrimidine-2,4-diamine;
II-5 N4-(benzo[d]oxazol-2(3H)-on-5-yl)-N2-(6-(4-tert-butyloxycarbonylpiperazin-1-yl)pyridin-3-yl)-5-methylpyrimidine-2,4-diamine;
II-6 N4-(benzoxazol-2 (3H)-on-5-yl)-N2-(6-(4-methylpiperidin-1-yl)pyridin-3-yl)-5-methylpyrimidine-2,4-diamine;
II-7 N2-(6-(4-methylpiperazin-1-yl)pyridin-3-yl)-N4-(3-isopropylbenzo[d]oxazol-2(3H)-on-5-yl)-5-methylpyrimidine-2,4-diamine;
II-8 N4-(benzo[d]oxazol-2(3H)-on-5-yl)-N2-(6-(4-trifluoromethoxycarbonylpiperazin-1-yl)pyridin-3-yl)-5-methylpyrimidine-2,4-diamine;
II-9 N4-(benzo[d]oxazol-2(3H)-on-5-yl)-N2-(6-(4-methoxycarbonylpiperazin-1-yl)pyridin-3-yl)-5-methylpyrimidine-2,4-diamine;
II-10 N4-(benzo[d]oxazol-2(3H)-on-5-yl)-N2-(6-(piperazin-1-yl)pyridin-3-yl)-5-methylpyrimidine-2,4-diamine;
II-11 N4-(benzo[d]oxazol-2(3H)-on-5-yl)-N2-(6-(3-methyl-4-tert-butoxycarbonylpiperazin-1-yl)pyridin-3-yl)-5-methylpyrimidine-2,4-diamine;
II-12 N4-(benzo[d]oxazol-2(3H)-on-5-yl)-N2-(6-(3-methylpiperazin-1-yl)pyridin-3-yl)-5-methylpyrimidine-2,4-diamine;
II-13 N4-(benzoxazolin-2-on-5-yl)-N2-[2-(4-methylpiperazin-1-yl)pyridin-5-yl]-5-methylpyrimidine-2,4-diamine;
II-14 N4-(benzo[d]oxazol-2(3H)-on-5-yl)-N2-[2-(4-methylpiperazin-1-yl)pyridin-5-yl]-5-fluoropyrimidine-2,4-diamine;
II-15 N4-(benzimidazolin-2-on-5-yl)-N2-[2-(4-methylpiperazin-1-yl)pyridin-5-yl]-5-methylpyrimidine-2,4-diamine;
II-16 N4-(benzimidazolin-2-on-5-yl)-N2-[2-(4-methylpiperazin-1-yl)pyridin-5-yl]-5-fluoropyrimidine-2,4-diamine;
II-17 N4-(benzo[d]oxazol-2(3H)-on-5-yl)-N2-((2-morpholinyl)pyridin-5-yl)-5-methylpyrimidine-2,4-diamine;
II-18 N4-(benzo[d]oxazol-2(3H)-on-5-yl)-N2-((2-morpholinyl)pyridin-5-yl)-5-fluoropyrimidine-2,4-diamine;
II-19 N4-(benzimidazolin-2-on-5-yl)-N2-((2-morpholinyl)pyridin-5-yl)-5-fluoropyrimidine-2,4-diamine;
II-20 N4-(1,3-dimethylbenzimidazolin-2-on-5-yl)-N2-[2-(4-methylpiperazino)pyridin-5-yl]-5-methylpyrimidine-2,4-diamine;
II-21 N4-(1,3-dimethylbenzimidazolin-2-on-5-yl)-N2-[2-(4-methylpiperazino)pyridin-5-yl]-5-fluoropyrimidine-2,4-diamine;
II-22 N2-(6-(4-methylpiperazin-1-yl)pyridin-3-yl)-N4-(3-methyl-2-oxo-2,3-dihydrobenzo[d]oxazol-5-yl)-5-methylpyrimidine-2,4-diamine;
II-23 N2-(6-(4-methylpiperazin-1-yl)pyridin-3-yl)-N4-(3-methyl-2-oxo-2,3-dihydrobenzo[d]oxazol-6-yl)-5-methylpyrimidine-2,4-diamine trifluoroacetate salt;
II-24 N2-(6-(4-methylpiperazin-1-yl)pyridin-3-yl)-N4-(3-methyl-2-oxo-2,3-dihydrobenzo[d]oxazol-5-yl)-5-fluoropyrimidine-2,4-diamine;
II-25 6-(5-methyl-2-(6-(4-methylpiperazin-1-yl)pyridin-3-ylamino)pyrimidin-4-ylamino)benzo[d]oxazol-2(3H)-one;
II-26 N4-(benzoxazolin-2-on-5-yl)-N2-[3-methyl-2-(4-methylpiperazin-1-yl)pyridin-5-yl]-5-methylpyrimidine-2,4-diamine;
II-27 N4-(benzimidazolin-2-on-5-yl)-N2-[3-methyl-2-(4-methylpiperazin-1-yl)pyridin-5-yl]-5-methylpyrimidine-2,4-diamine;
II-28 N4-(benzoxazolin-2-on-5-yl)-N2-[3-methyl-2-((1S,4S)-5-methyl-2,5-diazabicyclo[2.2.1]heptan-2-yl)pyridin-5-yl]-5-methylpyrimidine-2,4-diamine;
II-29 N4-(benzoxazolin-2-on-5-yl)-N2-[3-methyl-2-((1 S,4S)-5-methyl-2,5-diazabicyclo[2.2.1]heptan-2-yl)pyridin-5-yl]-5-fluoropyrimidine-2,4-diamine;
II-30 N4-(benzoxazolin-2-on-5-yl)-N2-[3-methyl-2-(4-methylpiperazin-1-yl)pyridin-5-yl]-5-fluoropyrimidine-2,4-diamine;
II-31 N4-(benzoxazolin-2-on-5-yl)-N2-[2-((1S,4S)-5-methyl-2,5-diazabicyclo[2.2.1]heptan-2-yl)pyridin-5-yl]-5-methylpyrimidine-2,4-diamine;
II-32 N4-(benzoxazolin-2-on-5-yl)-N2-[2-((1S,4S)-2-oxa-5-azabicyclo[2.2.1]heptan-5-yl)pyridin-5-yl]-5-methylpyrimidine-2,4-diamine;
II-33 N4-(benzoxazolin-2-on-5-yl)-N2-[2-((1 S,4S)-5-methyl-2,5-diazabicyclo[2.2.1]heptan-2-yl)pyridin-5-yl]-5-fluoropyrimidine-2,4-diamine;
II-34 N4-(benzoxazolin-2-on-5-yl)-N2-[2-((1 S,4S)-2-oxa-5-azabicyclo[2.2.1]heptan-5-yl)pyridin-5-yl]-5-fluoropyrimidine-2,4-diamine;
II-35 N4-(benzo[d]oxazol-2(3H)-on-5-yl)-N2-[2-(1-methylpiperidin-4-yl)aminopyridin-5-yl]-5-methylpyrimidine-2,4-diamine;
II-36 N4-(benzo[d]oxazol-2(3H)-on-5-yl)-N2-[2-(1H-piperidin-4-yl)aminopyridin-5-yl]-5-methylpyrimidine-2,4-diamine;
II-37 N4-(benzo[d]oxazol-2(3H)-on-5-yl)-N2-[2-(8-methyl-8-aza-bicyclo[3.2.1]oct-3-yl)aminopyridin-5-yl)]-5-methylpyrimidine-2,4-diamine;
II-38 N4-(benzo[d]oxazol-2(3H)-on-5-yl)-N2-(4-(8-methyl-2,8-diazabicyclo[3.2.1]octan-2-yl)phenyl)-5-methylpyrimidine-2,4-diamine;
II-39 N4-(benzo[d]oxazolin-2(3H)-on-5-yl)-N2-[3-trifluoromethyl-2-(4-methylpiperazin-1-yl)pyridin-5-yl]-5-methylpyrimidine-2,4-diamine;
II-40 N4-(benzoxazolin-2-on-5-yl)-N2-[3-fluoro-2-((1 S,4S)-5-methyl-2,5-diazabicyclo[2.2.1]heptan-2-yl)pyridin-5-yl]-5-methylpyrimidine-2,4-diamine;
II-41 (S)-2-Methyl-4-{5-[5-methyl-4-(2-oxo-2,3-dihydro-benzooxazol-5-ylamino)-pyrimidin-2-ylamino]-pyridin-2-yl}-piperazine-1-carboxylic acid tert-butyl ester;
II-42 5-[5-Methyl-2-(pyridin-3-ylamino)-pyrimidin-4-ylamino]-3H-benzooxazol-2-one;
II-43 5-[2-(4-(6-Methanesulfonyl-pyridin-3-ylamino)-5-methyl-pyrimidin-4-ylamino]-3H-benzooxazol-2-one;
II-44 5-{5-Methyl-2-[6-((S)-3-methyl-piperazin-1-yl)-pyridin-3-ylamino]-pyrimidin-4-ylamino}-3H-benzooxazol-2-one;
II-45 5-{5-Methyl-2-[6-(piperazine-1-carbonyl)-pyridin-3-ylamino]-pyrimidin-4-ylamino}-3H-benzooxazol-2-one;
II-46 5-{2-[6-(4-Cyclopropylmethyl-piperazine-1-carbonyl)-pyridin-3-ylamino]-5-methyl-pyrimidin-4-ylamino}-3H-benzooxazol-2-one;
II-47 5-{2-[6-(4-Isobutyl-piperazine-1-carbonyl)-pyridin-3-ylamino]-5-methyl-pyrimidin-4-ylamino}-3H-benzooxazol-2-one;
II-48 5-{3-Fluoro-5-[5-methyl-4-(2-oxo-2,3-dihydro-benzooxazol-5-ylamino)-pyrimidin-2-ylamino]-pyridin-2-yl}-hexahydro-pyrrolo[3,4-c]pyrrole-2-carboxylic acid tert-butyl ester;
II-49 5-{3-Fluoro-5-[5-methyl-4-(2-oxo-2,3-dihydro-benzooxazol-5-ylamino)-pyrimidin-2-ylamino]-pyridin-2-yl}-2,5-diaza-bicyclo[2.2.1]heptane-2-carboxylic acid tert-butyl ester;
II-50 5-{2-[5-Fluoro-6-(hexahydro-pyrrolo[3,4-c]pyrrol-2-yl)-pyridin-3-ylamino]-5-methyl-pyrimidin-4-ylamino}-3H-benzooxazol-2-one;
II-51 5-{2-[6-(2,5-Diaza-bicyclo[2.2.1]hept-2-yl)-5-fluoro-pyridin-3-ylamino]-5-methyl-pyrimidin-4-ylamino}-3H-benzooxazol-2-one;
II-52 5-{2-[6-(2,5-Cyclopropylmethyl-hexahydro-pyrrolo[3,4-c]pyrrol-2-yl)-5-fluoro-pyridin-3-ylamino]-5-methyl-pyrimidin-4-ylamino}-3H-benzooxazol-2-one;
II-53 5-{2-[6-(2,5-Cyclopropanecarbonyl-hexahydro-pyrrolo[3,4-c]pyrrol-2-yl)-5-fluoro-pyridin-3-ylamino]-5-methyl-pyrimidin-4-ylamino}-3H-benzooxazol-2-one;
II-54 5-{2-[6-(2,5-Cyclopropylmethyl-2,5-diaza-bicyclo[2.2.1]hept-2-yl)-5-fluoro-pyridin-3-ylamino]-5-methyl-pyrimidin-4-ylamino}-3H-benzooxazol-2-one;
II-55 (R)-5-(2-(6-(3 , 4- dimethylpiperazin-1-yl)pyridin-3-ylamino)-5-methylpyrimidin-4-ylamino)benzo[d]oxazol2(3H)-one;
II-56 (R)-5-(2-(6-(4-(cyclopropylmethyl)-3-methylpiperazin-1-yl)pyridin-3-ylamino)-5-methylpyrimidin-4-ylamino)benzo[d]oxazol-2(3H)-one;
II-57 (R)-5-(5-methyl-2-(6-(3-methyl-4-(2,2,2-trifluoroacetyppiperazin-1-yl)pyridin-3-ylamino)pyrimidin-4-ylamino)benzo[d]oxazol2(3H)-one;
II-58 (R)-diethyl 2-methyl-4-(5-(5-methyl-4-(2-oxo-2,3-dihydrobenzo[d]oxazol5-ylamino)pyrimidin-2-ylamino)pyridin-2-yl)piperazin-1-ylphosphonate;
II-59 5-(2-(6-(4,4-difluoropiperidin-1-yl)pyridin-3-ylamino)-5-methylpyrimidin-4-ylamino)benzo[d]oxazol-2(3H)-one;
II-60 5-(2-(6-(4,4-dimethylpiperidin-1-yl)pyridin-3-ylamino)-5-methylpyrimidin-4-ylamino)benzo[d]oxazol2(3H)-one;
II-61 5-(2-(6-(3,8-diaza-bicyclo[3.2.1]octan-3-yl)-5-methylpyridin-3-ylamino)-5-methylpyrimidin-4-ylamino)benzo[d]oxazol2(3H)-one;
II-62 5-(5-methyl-2-(5-methyl-6-(8-acetyl)-3,8-diaza-bicyclo[3.2.1]octan-3-yl)pyridin-3-ylamino)pyrimidin-4-ylamino)benzo[d]oxazol2(3H)-one;
II-63 5-(5-methyl-2-(5-methyl-6-(8-(2,2,2-trifluoroacetyl)-3,8-diaza-bicyclo[3.2.1]octan-3-yl)pyridin-3-ylamino)pyrimidin-4-ylamino)benzo[d]oxazol2(3H)-one;
II-64 5-(5-methyl-2-(5-methyl-6-(8-methyl-3,8-diaza-bicyclo[3.2.1]octan-3-yl)pyridin-3-ylamino)pyrimidin-4-ylamino)benzo[d]oxazol2(3H)-one;
II-65 tert-butyl 3-(3-methyl-5-(5-methyl-4-(2-oxo-2,3-dihydrobenzo[d]oxazol-5-ylamino)pyrimidin-2-ylamino)pyridin-2-yl)-8-azabicyclo[3.2.1]octane-8-carboxylate;
II-66 5-(2-(6-(8-aza-bicyclo[3.2.1]octan-3-yl)-5-methylpyridin-3-ylamino)-5-methylpyrimidin-4-ylamino)benzo[d]oxazol-2(3H)-one;
II-67 5-(2-(6-(8-(cyclopropylmethyl)-8-aza-bicyclo[3.2.1]octan-3-yl)-5-methylpyridin-3-ylamino)-5-methylpyrimidin-4-ylamino)benzo[d]oxazol2(3H)-one;
II-68 methyl 3-(3-methyl-5-(5-methyl-4-(2-oxo-2,3-dihydrobenzo[d]oxazol5-ylamino)pyrimidin-2-ylamino)pyridin-2-yl)-8-azabicyclo[3.2.1]octane-8-carboxylate;
II-69 5-(5-methyl-2-(5-methyl-6-(8-(2,2,2-trifluoroacetyl)-8-aza-bicyclo[3.2.1]octan-3-yl)pyridin-3-ylamino)pyrimidin-4-ylamino)benzo[d]oxazol2(3H)-one;
II-70 (R)-5-(2-(6-(4-isopropyl-3-methylpiperazin-1-yl)pyridin-3-ylamino)-5-methylpyrimidin-4-ylamino)benzo[d]oxazol-2(3H)-one;
II-71 5-(5-methyl-2-(6-(pyrrolidin-1-yl)pyridin-3-ylamino)pyrimidin-4-ylamino)benzo[d]oxazol-2 (3H)-one;
II-72 7-methyl-5-(5-methyl-2-(6-(4-methylpiperazin-1-yl)pyridin-3-ylamino)pyrimidin-4-ylamino)benzo[d]oxazol-2(3H)-one;
II-73 7-methyl-5-(5-methyl-2-(6-morpholinopyridin-3-ylamino)pyrimidin-4-ylamino)benzo[d]oxazol-2(3H)-one;
II-74 5-(2-(6-(cyclopropylmethylamino)pyridin-3-ylamino)-5-methylpyrimidin-4-ylamino)benzo[d]oxazol-2(3H)-one;
II-75 7-fluoro-5-(5-methyl-2-(6-(4-methylpiperazin-1-yl)pyridin-3-ylamino)pyrimidin-4-ylamino)benzo[d]oxazol-2(3H)-one;
II-76 7-fluoro-5-(5-methyl-2-(6-morpholinopyridin-3-ylamino)pyrimidin-4-ylamino)benzo[d]oxazol-2(3H)-one;
II-77 5-(2-(5-bromopyridin-3-ylamino)-5-methylpyrimidin-4-ylamino)benzo[d]oxazol-2(3H)-one;
II-79 N-(5-(5-methyl-4-(3-methyl-2-oxo-2,3-dihydrobenzoxazol-5-ylamino)pyrimidin-2-ylamino)pyridin-2-yl)methanesulfonamide;
II-80 5-(2-(6-(3-(dimethylamino)pyrrolidin-1-yl)pyridin-3-ylamino)-5-methylpyrimidin-4-ylamino)benzo[d]oxazol-2(3H)-one;
II-81 N-(1-(5-(5-methyl-4-(2-oxo-2,3-dihydrobenzo[d]oxazol-5-ylamino)pyrimidin-2-ylamino)pyridin-2-yl)pyrrolidin-3-yl)acetamide;
II-82 5-(2-(6-(3-(diethylamino)pyrrolidin-1-yl)pyridin-3-ylamino)-5-methylpyrimidin-4-ylamino)benzo[d]oxazol-2(3H)-one;
II-83 2,2,2-trifluoro-N-(1-(5-(5-methyl-4-(2-oxo-2,3-dihydrobenzoxazol-5-ylamino)pyrimidin-2-ylamino)pyridin -2-yl)pyrrolidin-3-yl)acetamide;
II-84 5-(5-methyl-2-(6-(3-morpholinopyrrolidin-1-yl)pyridin-3-ylamino)pyrimidin-4-ylamino)benzo[d]oxazol-2(3H)-one;
II-85 5-(2-(6-(4-methylpiperazin-1-yl)pyridin-3-ylamino)-5-(trifluoromethyl)pyrimidin-4-ylamino)benzo[d]oxazol-2(3H)-one;
II-86 tert-butyl 1-(5-(5-methyl-4-(2-oxo-2,3-dihydrobenzo[d]oxazol-5-ylamino)pyrimidin-2-ylamino)pyridin-2-yl)pyrrolidin-3-ylcarbamate;
II-87 (S)-tert-butyl methyl(1-(5-(5-methyl-4-(2-oxo-2,3-dihydrobenzo[d]oxazol-5-ylamino)pyrimidin-2-ylamino)pyridin-2-yl)piperidin-3-yl)carbamate;
II-88 (R)-5-(5-methyl-2-(6-(3-(methylamino)piperidin-1-yl)pyridin-3-ylamino)pyrimidin-4-ylamino)benzo[d]oxazol-2(3H)-one;
II-89 (R)-5-(2-(6-(3-(dimethylamino)pyrrolidin-1-yl)pyridin-3-ylamino)-5-methylpyrimidin-4-ylamino)benzo[a]oxazol-2(3H)-one;
II-90 (S)-5-(2-(6-(3-(dimethylamino)pyrrolidin-1-yl)pyridin-3-ylamino)-5-methylpyrimidin-4-ylamino)benzo[d]oxazol-2(3H)-one;
II-91 (R)-tert-butyl methyl (1-(5-(5-methyl-4-(2-oxo-2,3-dihydrobenzo[d]oxazol-5-ylamino)pyrimidin-2-ylamino)pyridin-2-yl)piperidin-3-yl)carbamate;
II-92 (R)-5-(5-methyl-2-(6-(3-(methylamino)piperidin-1-yl)pyridin-3-ylamino)pyrimidin-4-ylamino)benzo[d]oxazol-2(3H)-one;
II-94 5-(2-(6-(3-(cyclopropylmethylamino)pyrrolidin-1-yl)pyridin-3-ylamino)-5-methylpyrimidin-4-ylamino)benzo[d]oxazol-2(3H)-one;
II-95 (S)-5-(2-(6-((1-benzylpiperidin-3-yl)(methyl)amino)pyridin-3-ylamino)-5-methylpyrimidin-4-ylamino)benzo[d]oxazol-2(3H)-one;
II-96 1-ethyl-3-(1-(5-(5-methyl-4-(2-oxo-2,3-dihydrobenzo[d]oxazol-5-ylamino)pyrimidin-2-ylamino)pyridin-2-yl)pyrrolidin-3-yl)urea;
II-97 1-tert-butyl-3-(1-(5-(5-methyl-4-(2-oxo-2,3-dihydrobenzo[d]oxazol-5-ylamino)pyrimidin-2-ylamino)pyridin-2-yl)pyrrolidin-3-yl)urea;
II-98 1-benzyl-3-(1-(5-(5-methyl-4-(2-oxo-2,3-dihydrobenzo[d]oxazol-5-ylamino)pyrimidn-2-ylamino)pyridin-2-yl)pyrrolidin-3-yl)urea;
II-99 (S)-5-(2-(2-(6-(1-benzylpiperidin-3-ylamino)pyridin-3-ylamino)-5-methylpyrimidin-4-ylamino)benzo[d]oxazol-2(3H)-one;
II-100 (S)-5-(2-(6-((1-benzylpiperidin-3-yl)(methyl)amino)pyridin-3-ylamino)-5-methylpyrimidin-4-ylamino)benzo[d]oxazol-2(3H)-one;
II-101 N-(1-(5-(5-methyl-4-(2-oxo-2,3-dihydrobenzo[d]oxazol-5-ylamino)pyrimidin-2-ylamino)pyridin-2-yl)pyrrolidin-3-yl)cyclopropanecarboxamide;
II-102 N-(1-(5-(5-methyl-4-(2-oxo-2,3-dihydrobenzo[d]oxazol-5-ylamino)pyrimidin-2-ylamino)pyridin-2-yl)pyrrolidin-3-yl)pivalamide;
II-103 (S)-5-(5-methyl-2-(6-(methyl(piperidin-3-yl)amino)pyridin-3-ylamino)pyrimidin-4-ylamino)benzo[d]oxazol-2(3H)-one;
II-104 (S)-5-(5-methyl-2-(6-(piperidin-3-ylamino)pyridin-3-ylamino)pyrimidin-4-ylamino)benzo[d]oxazol-2(3H)-one;
II-105 (S)-5-(2-(6-(1-benzylpiperidin-3-ylamino)pyridin-3-ylamino)-5-methylpyrimidin-4-ylamino)benzo[d]oxazol-2(3H)-one;
II-106 (R)-5-(2-(6-((1-benzylpiperidin-3-yl)(methyl)amino)pyridin-3-ylamino)-5-methylpyrimidin-4-ylamino)benzo[d]oxazol-2(3H)-one;
II-107 (R)-5-(5-methyl-2-(6-(piperidin-3-ylamino)pyridin-3-ylamino)pyrimidin-4-ylamino)benzo[d]oxazol-2(3H)-one;
II-108 (R)-5-(5-methyl-2-(6-(methyl(piperidin-3-yl)amino)pyridin-3-ylamino)pyrimidin-4-ylamino)benzo[d]oxazol-2(3H)-one;
II-109 N4-(benzo[d]oxazolin-2(3H)-one-5-yl)-5-methyl-N2-[2-((1 S,4S)-5-methyl-2,5-diazabicyclo[2.2.1]heptan-2-yl)-3-trifluoromethylpyridine-5-yl]-2,4-pyrimidinediamine;
II-110 N4-(benzo[d]oxazolin-2(3H)-one-5-yl)-N2-[2-(4-ethylpiperazin-1-yl)-3-trifluoromethylpyridine-5-yl]-5-methyl-2,4-pyrimidinediamine;
II-111 N4-(benzo[d]oxazolin-2(3H)-one-5-yl)-N2-[3-fluoro-2-(4-methylpiperazin-1-yl)pyridine-5-yl]-5-methyl-2,4-pyrimidinediamine;
II-112 N4-(benzo[d]oxazolin-2(3H)-one-5-yl)-N2-{2-[(8 S)-1,4-diazabicyclo[4.3.0]nonane-1-yl]-3-fluoropyridine-5-yl}-5-methyl-2,4-pyrimidinediamine;
II-113 N4-(benzo[d]oxazolin-2(3H)-one-5-yl)-N2-{2-[(8R)-1,4-diazabicyclo[4.3.0]nonane-1-yl]-3-fluoropyridine-5-yl}-5-methyl-2,4-pyrimidinediamine;
II-114 N4-(benzo[d]oxazolin-2(3H)-one-5-yl)-N2-[2-(4-ethylpiperazin-1-yl)-3-fluoropyridine-5-yl]-5-methyl-2,4-pyrimidinediamine;
II-115 N4-(benzo[d]oxazolin-2(3H)-one-5-yl)-N2-[3-cyano-24(1 S,4S)-5-methyl-2,5-diazabicyclo[2.2.1]heptan-2-yl)pyridine-5-yl]-5-methyl-2,4-pyrimidinediamine;
II-116 N2-[3-chloro-2-(4-methylpiperazino)pyridin-5-yl]-5-methyl-N4-(2-oxo-2,3-dihydro-1,3-benzoxazol-5-yl)-2,4-pyrimidinediamine;
II-117 5-methyl-N4-(2-oxo-2,3-dihydro-1,3-benzoxazol-5-yl)-N2-[2-(1,3,5-trimethyl-3 ,7-diazabicyclo[3.3.1]nonan-7-yl)pyridin-5-yl]-2,4-pyrimidinediamine;
II-118 N2-[3-chloro-2-(3-ethyl-3,7-diazabicyclo[3.3.0]octan-7-yl)pyridin-5-yl]-5-methyl-N4-(2-oxo-2,3-dihydro-1,3-benzoxazol-5-yl)-2,4-pyrimidinediamine;
II-119 N2-[2-(3-ethyl-3,7-diazabicyclo[3.3.0]octan-7-yl)-3-trifluoromethylpyridin-5-yl]-5-methyl-N4-(2-oxo-2,3-dihydro-1,3-benzoxazol-5-yl)-2,4-pyrimidinediamine;
II-120 5-methyl-N2-[2-(3-methyl-3,7-diazabicyclo[3.3.0]octan-7-yl)pyridin-5-yl]-N4-(2-oxo-2,3-dihydro-1,3-benzoxazol-5-yl)-2,4-pyrimidinediamine;
II-121 5-methyl-N2-[2-(octahydroisoindol-1-yl)pyridin-5-yl]-N4-(2-oxo-2,3-dihydro-1,3-benzoxazol-5-yl)-2,4-pyrimidinediamine;
II-122 N2-[3-chloro-2-(octahydroisoindol-1-yl)pyridin-5-yl]-5-methyl-N4-(2-oxo-2,3-dihydro-1,3-benzoxazol-5-yl)-2,4-pyrimidinediamine;
II-123 N2-(2-methoxypyridin-5-yl)-5-methyl-N4-(2-oxo-2,3-dihydro-1,3-benzoxazol-5-yl)-2,4-pyrimidinediamine;
II-124 N2-[2-(S-1,4-diazabicyclo[4.3.0]nonan-4-yl)-3-trifluoromethylpyridin-5-yl]-5-methyl-N4-(2-oxo-2,3-dihydro-1,3-benzoxazol-5-yl)-2,4-pyrimidinediamine;
II-125 N2-[2-(1,4-diazabicyclo[3.2.2]nonan-4-yl)-3-fluoropyridin-5-yl]-5-methyl-N4-(2-oxo-2,3-dihydro-1,3-benzoxazol-5-yl)-2,4-pyrimidinediamine;
II-126 N2-[2-(4R-hydroxy-2-methylidene-pyrrolidin-1-yl)pyridin-5-yl]-5-methyl-N4-(2-oxo-2,3-dihydro-1,3-benzoxazol-5-yl)-2,4-pyrimidinediamine;
II-127 5-methyl-N4-(2-oxo-2,3-dihydro-1,3-benzoxazol-5-yl)-N2-[2-(cis-3,4,5-trimethylpiperazino)pyridin-5-yl]-2,4-pyrimidinediamine;
II-128 N2-[2-(1,4-diazabicyclo[4.4.0]decan-4-yl)pyridin-5-yl]-5-methyl-N4-(2-oxo-2,3-dihydro-1,3-benzoxazol-5-yl)-2,4-pyrimidinediamine;
II-129 5-methyl-N4-(2-oxo-2,3-dihydro-1,3-benzoxazol-5-yl)-N2-[2-(trans-2,4,5-trimethylpiperazino)pyridin-5-yl]-2,4-pyrimidinediamine;
II-130 N2-[2-(trans-2,5-dimethylpiperazino)pyridin-5-yl]-5-methyl-N4-(2-oxo-2,3-dihydro-1,3-benzoxazol-5-yl)-2,4-pyrimidinediamine;
II-131 N2-[2-(cis-3,5-dimethylpiperazino)pyridin-5-yl]-5-methyl-N4-(2-oxo-2,3-dihydro-1,3-benzoxazol-5-yl)-2,4-pyrimidinediamine;
II-132 N2-[2-(R-1,4-diazabicyclo[4.3.0]nonan-4-yl)pyridin-5-yl]-5-methyl-N4-(2-oxo-2,3-dihydro-1,3-benzoxazol-5-yl)-2,4-pyrimidinediamine;
II-133 5-methyl-N2-[2-(7-methyl-2,7-diazaspiro[4 .4]nonan-2-yl)pyridin-5-yl]-N4-(2-oxo-2,3-dihydro-1,3-benzoxazol-5-yl)-2,4-pyrimidinediamine;
II-134 5-methyl-N2-[2-(3 S-methylmorpholino)pyridin-5-yl]-N4-(2-oxo-2,3-dihydro-1,3-benzoxazol-5-yl)-2,4-pyrimidinediamine;
II-135 5-methyl-N2-[2-(2R-methylmorpholino)pyridin-5-yl]-N4-(2-oxo-2,3-dihydro-1,3-benzoxazol-5-yl)-2,4-pyrimidinediamine;
II-136 N2-[2-(4-isopropylpiperazino)pyridin-5-yl]-5-methyl-N4-(2-oxo-2,3-dihydro-1,3-benzoxazol-5-yl)-2,4-pyrimidinediamine;
II-137 N2-[2-(3-N,N-dimethylamino-8-azabicyclo[3.2.1]octan-8-yl)pyridin-5-yl]-5-methyl-N4-(2-oxo-2,3-dihydro-1,3-benzoxazol-5-yl)-2,4-pyrimidinediamine;
II-138 5-methyl-N2-[2-(2 S-methylmorpholino)pyridin-5-yl]-N4-(2-oxo-2,3-dihydro-1,3-benzoxazol-5-yl)-2,4-pyrimidinediamine;
II-139 5-methyl-N2-{2-[(1R,4R)-2-oxa-5-azabicyclo[2 .2 . 1]heptan-5-yl]pyridin-5-yl}-N4-(2-oxo-2,3-dihydro-1,3-benzoxazol-5-yl)-2,4-pyrimidinediamine;
II-140 N2-(2,3-dimethoxypyridin-5-yl)-5-methyl-N4-(2-oxo-2,3-dihydro-1,3-benzoxazol-5-yl)-2,4-pyrimidinediamine;
II-141 N2-(2-methoxy-3-methylpyridin-5-yl)-5-methyl-N4-(2-oxo-2,3-dihydro-1,3-benzoxazol-5-yl)-2,4-pyrimidinediamine;
II-142 N2-[2-(2-hydroxy)ethoxypyridin-5-yl]-5-methyl-N4-(2-oxo-2,3-dihydro-1,3-benzoxazol-5-yl)-2,4-pyrimidinediamine;
II-143 N2-[4-methyl-2-(4-methylpiperazino)pyridin-5-yl]-5-methyl-N4-(2-oxo-2,3-dihydro-1,3-benzoxazol-5-yl)-2,4-pyrimidinediamine;
II-144 N2-(2-isopropoxypyridin-5-yl)-5-methyl-N4-(2-oxo-2,3-dihydro-1,3-benzoxazol-5-yl)-2,4-pyrimidinediamine;
II-145 N2-[2-(2-methoxy)ethoxypyridin-5-yl]-5-methyl-N4-(2-oxo-2,3-dihydro-1,3-benzoxazol-5-yl)-2,4-pyrimidinediamine;
II-146 N2-[2-(1-aminocarbonyl-1-methypethoxypyridin-5-yl]-5-methyl-N4-(2-oxo-2,3-dihydro-1,3-benzoxazol-5-yl)-2,4-pyrimidinediamine;
II-147 N2-(2-methoxy-3-trifluoromethylpyridin-5-yl)-5-methyl-N4-(2-oxo-2,3-dihydro-1,3-benzoxazol-5-yl)-2,4-pyrimidinediamine;
II-148 N2-[2-(3-hydroxy)propoxypyridin-5-yl]-5-methyl-N4-(2-oxo-2,3-dihydro-1,3-benzoxazol-5-yl)-2,4-pyrimidinediamine;
II-149 N2-[2-(3-methoxy)propoxypyridin-5-yl]-5-methyl-N4-(2-oxo-2,3-dihydro-1,3-benzoxazol-5-yl)-2,4-pyrimidinediamine;
II-150 5-(2-(6-(1,4-diazabicyclo[3.2.2]nonan-4-yl)-5-chloropyridin-3-ylamino)-5-methylpyrimidin-4-ylamino)benzo[d]oxazol-2(3H)-one;
II-151 5-[5-Methyl-4-(2-oxo-2,3-dihydro-benzooxazol-5-ylamino)-pyrimidin-2-ylamino]-pyridine-2-carboxylic acid cyclobutylamide;
II-152 N2-(5-methoxypyridin-3-yl)-5-methyl-N4-(2-oxo-2,3-dihydro-1,3-benzoxazol-5-yl)-2,4-pyrimidinediamine;
II-153 N2-(2,3-dimethylpyridin-5-yl)-5-methyl-N4-(2-oxo-2,3-dihydro-1,3-benzoxazol-5-yl)-2,4-pyrimidinediamine;
III-1 N4-(benzo[d]oxazol-2(3H)-on-5-yl)-N2-(isoindolin-5-yl)-5-methylpyrimidine-2,4-diamine;
III-2 N4-(benzo[d]oxazol-2(3H)-on-5-yl)-N2-(2Hydroxyisoindolin-5-yl)-5-methylpyrimidine-2,4-diamine;
III-3 N4-(benzo[d]oxazol-2(3H)-on-5-yl)-N2-(2-tert-butoxoxycarbonylisoindolin-5-yl)-5-methylpyrimidine-2,4-diamine;
III-4 N4-(benzo[d]oxazol-2(3H)-on-5-yl)-N2-(2-methylisoindolin-5-yl)-5-methylpyrimidine-2,4-diamine;
III-5 N4-(benzo[d]oxazol-2(3H)-on-5-yl)-N2-(2-ethylisoindolin-5-yl)-5-methylpyrimidine-2,4-diamine;
III-6 N4-(benzo[d]oxazol-2(3H)-on-5-yl)-N2-(2-n-propylisoindolin-5-yl)-5-methylpyrimidine-2,4-diamine;
III-7 N4-(benzo[d]oxazol-2(3H)-on-5-yl)-N2-(2-cyclopropylmethylylisoindolin-5-yl)-5-methylpyrimidine-2,4-diamine;
III-8 N4-(benzo[d]oxazol-2(3H)-on-5-yl)-N2-(2-isobutylisoindolin-5-yl)-5-methylpyrimidine-2,4-diamine;
III-9 N4-(benzo[d]oxazol-2(3H)-on-5-yl)-N2-(2-isopentylisoindolin-5-yl)-5-methylpyrimidine-2,4-diamine;
III-10 N4-(benzo[d]oxazol-2(3H)-on-5-yl)-N2-(2-cyclopentylmethylisoindolin-5-yl)-5-methylpyrimidine-2,4-diamine;
III-11 N4-(benzo[d]oxazol-2(3H)-on-5-yl)-N2-(2-(bicyclo[2.2.1]heptan-2-ylmethyl)isoindolin-5-yl)-5-methylpyrimidine-2,4-diamine;
III-12 5-[2-(2-Acetyl-2,3-dihydro-1H-isoindol-5-ylamino)-5-methyl-pyrimidin-4-ylamino]-3H-benzooxazol-2-one;
III-13 N-{2[2(2,2-Dimethyl-propionyl)-2,3-dihydro-1H-isoindol-5-ylamino]-5-methyl-pyrimidin-4-yl}-N-[3-(2,2-dimethyl-propionyl)-2-oxo-2,3-dihydro-benzooxazol-5-yl]-2,2-dimethyl-propionamide;
III-14 5-[2-(2-Methane sulfonyl-2,3-dihydro-1H-isoindol-5-ylamino)-5-methyl-pyrimidin-4-ylamino]-3H-benzooxazol-2-one;
IV-1 N4-(benzo[d]oxazol-2(3H)-on-5-yl)-N2-(7-(pyrrolidin-1-yl)-6,7, 8,9-tetrahydro-5H-benzo[7]annulen-2-yl)-5-methylpyrimidine-2,4-diamine;
IV-2 N4-(benzo[d]oxazol-2(3H)-on-5-yl)-N2-(6,7, 8,9-tetrahydro-5H-benzo[7]annulen-5-on-3-yl)-5-methylpyrimidine-2,4-diamine;
IV-3 N4-(benzo[d]oxazol-2(3H)-on-5-yl)-N2-(6-(4-methylpiperazin-1-yl)pyridazin-3-yl)-5-methylpyrimidine-2,4-diamine;
IV-4 N4-(benzo[d]oxazol-2(3H)-on-5-yl)-N2-(1 H-indazol-6-yl)-5-methylpyrimidine-2,4-diamine;
IV-5 N4-(benzo[d]oxazol-2(3H)-on-5-yl)-N2-(1,2-benzisoxazol-6-yl)-5-methylpyrimidine-2,4-diamine;
IV-6 N4-(benzo[d]oxazol-2(3H)-on-5-yl)-N2-(1 H-indazol-5-yl)-5-methylpyrimidine-2,4-diamine;
IV-7 N4-(benzo[d]oxazol-2(3H)-on-5-yl)-N2-[2-(piperazino)pyridin-4-yl]-5-methylpyrimidine-2,4-diamine;
IV-8 N4-(benzo[d]oxazol-2(3H)-on-5-yl)-N2-[2-(4-methylpiperazino)pyridin-4-yl]-5-methylpyrimidine-2,4-diamine;
IV-9 N4-(benzo[d]oxazol-2(3H)-on-5-yl)-N2-(3-methyl-1,2-benzisoxazol-5-yl)-5-methylpyrimidine-2,4-diamine;
IV-10 (Z)-2-Methyl-9-[5-methyl-4-(2-oxo-2,3-dihydro-benzooxazol-5-ylamino)-pyrimidin-2-ylamino]-3,6-dihydro-2H-benzo[c] azocin-1-one;
IV-11 5-[2-(2,2-Difluoro-benzo[1,3]dioxol-4-ylamino)-5-methyl-pyrimidin-4-ylamino]-3H-benzooxazol-2-one;
IV-12 5-[2-(9-Isopropylamino-6,7,8,9-tetrahydro-5H-benzocyclohepten-2-ylamino)-5-methyl-pyrimidin-4-ylamino]-3H-benzooxazol-2-one;
IV-13 5-{2-[9-(3-Diethylamino-pyrrolidin-1-yl)-6,7,8,9-tetrahydro-5H-benzocyclohepten-2-ylamino]-5-methyl-pyrimidin-4-ylamino}-3H-benzooxazol-2-one;
IV-14 2-Methyl-9-[5-methyl-4-(2-oxo-2,3-dihydro-benzooxazol-5-ylamino)-pyrimidin-2-ylamino]-3,4,5,6-tetrahydro-2H-benzo[c] azocin-1-one;
IV-15 6-[5-Methyl-4-(2-oxo-2,3-dihydro-benzooxazol-5-ylamino)-pyrimidin-2-ylamino]-3,4-dihydro-2H-isoquinolin-1-one;
IV-16 5-[2-(2,2-Dioxo-1H-benzo[e] [1,3,4]oxathiazin-7-ylamino)-5-methyl-pyrimidin-4-ylamino]-3H-benzooxazol-2-one;
IV-17 5-[2-(2,2-Dimethyl-benzo[1,3]dioxol-5-ylamino)-5-methyl-pyrimidin-4-ylamino]-3H-benzooxazol-2-one;
IV-18 (Z)-5-(5-methyl-2-(1-oxo-2,3-dihydro-1H-benzo[c]azepin-7-ylamino)pyrimidin-4-ylamino)benzo[d]oxazol-2(3H)-one;
IV-19 (Z)-5-(5-methyl-2-(2-methyl-1-oxo-2,3-dihydro-1H-benzo[c]azepin-7-ylamino)pyrimidin-4-ylamino)benzo[d]oxazol-2(3H)-one;
IV-20 (Z)-5-(5-methyl-2-(2-methyl-1-oxo-2,3-dihydro-1H-benzo[c]azepin-7-ylamino)pyrimidin-4-ylamino)benzo[d]oxazol-2(3H)-one;
IV-21 5-(5-methyl-2-(2-methyl-1-oxo-2,3,4,5-tetrahydro-1H-benzo[c]azepin-7-ylamino)pyrimidin-4-ylamino)benzo[d]oxazol-2(3H)-one;
IV-22 5,5′-(5-methylpyrimidine-2,4-diyl)bis(azanediyl)dibenzo[d]oxazol-2(3H)-one
IV-23 5-(5-methyl-2-(2-oxo-2,3,4,5-tetrahydro-1H-benzo[b]azepin-8-ylamino)pyrimidin-4-ylamino)benzo[d]oxazol-2(3H)-one;
IV-24 5-(5-methyl-2-(2-oxo-1,2,3,4-tetrahydroquinolin-7-ylamino)pyrimidin-4-ylamino)benzo[a]oxazol-2(3H)-one;
IV-25 6-(5-methyl-4-(2-oxo-2,3-dihydrobenzo[d]oxazol-5-ylamino)pyrimidin-2-ylamino)-2H-benzo[b] [1,4]oxazin-3 (4H)-one;
IV-26 5-(2-(3,3-dimethyl-2-oxoindolin-6-ylamino)-5-methylpyrimidin-4-ylamino)benzo[a]oxazol-2(3H)-one;
IV-27 5-(5-methyl-2-(1-methyl-2-oxoindolin-5-ylamino)pyrimidin-4-ylamino)benzo[d]oxazol-2(3H)-one;
IV-28 5-(5-methyl-2-(1-methyl-2-oxo-2,3-dihydro-1H-benzo[a]imidazol-5-ylamino)pyrimidin-4-ylamino)benzo[d]oxazol-2(3H)-one;
IV-29 5-(5-methyl-2-(2-oxo-2,3-dihydro-1H-benzo[d]imidazol-5-ylamino)pyrimidin-4-ylamino)benzo[d]oxazol-2(3H)-one;
IV-30 5-(5-methyl-2-(1-methyl-2-oxo-2,3,4,5-tetrahydro-1H-benzo[b]azepin-8-ylamino)pyrimidin-4-ylamino)benzo[d]oxazol-2(3H)-one;
IV-31 5-(5-methyl-2-(2-oxo-2,3,4,5-tetrahydro-1H-benzo[b]azepin-7-ylamino)pyrimidin-4-ylamino)benzo[d]oxazol-2(3H)-one;
IV-32 7-(5-methyl-4-(2-oxo-2,3-dihydrobenzo[d]oxazol-5-ylamino)pyrimidin-2-ylamino)-2H-benzo[b] [1,4]oxazin-3 (4H)-one;
IV-33 5-(5-methyl-2-(2-oxo-1,2,3,4-tetrahydroquinolin-6-ylamino)pyrimidin-4-ylamino)benzo[a]oxazol-2(3H)-one;
IV-34 5-(5-methyl-2-(1-methyl-2-oxo-2,3,4,5-tetrahydro-1H-benzo[b]azepin-7-ylamino)pyrimidin-4-ylamino)benzo[d]oxazol-2(3H)-one;
IV-35 5-methyl-N2-(3,4-methylenedioxy)phenyl-N4-(2-oxo-2,3-dihydro-1,3-benzoxazol-5-yl)-2,4-pyrimidinediamine;
IV-36 N2-(2,2-difluoro-2H-1,3-benzodioxol-5-yl)-5-methyl-N4-(2-oxo-2,3-dihydro-1,3-benzoxazol-5-yl)-2,4-pyrimidinediamine;
IV-37 N2-(3,4-ethylenedioxy)phenyl-5-methyl-N4-(2-oxo-2,3-dihydro-1,3-benzoxazol-5-yl)-2,4-pyrimidinediamine;
IV-38 N2-(2,2-dimethyl-2H-1,3-benzodioxol-5-yl)-5-methyl-N4-(2-oxo-2,3-dihydro-1,3-benzoxazol-5-yl)-2,4-pyrimidinediamine;
IV-39 N2-[spiro(2,1′-cyclohexan)-1,3-benzodioxol-5-yl]-5-methyl-N4-(2-oxo-2,3-dihydro-1,3-benzoxazol-5-yl)-2,4-pyrimidinediamine;
IV-40 N2-(1,3-dimethyl-1H-pyrazolo[3,4-b]pyridin-5-yl)-5-methyl-N4-(2-oxo-2,3-dihydro-1,3-benzoxazol-5-yl)-2,4-pyrimidinediamine;
IV-41 5-methyl-N2-(1-methylindazol-6-yl)-N4-(2-oxo-2,3-dihydro-1,3-benzoxazol-5-yl)-2,4-pyrimidinediamine;
IV-42 5-methyl-N2-(1-methylindazol-5-yl)-N4-(2-oxo-2,3-dihydro-1,3-benzoxazol-5-yl)-2,4-pyrimidinediamine;
IV-43 5-methyl-N2-(3-methylisoxazolo[5,4-b]pyridin-5-yl)-N4-(2-oxo-2,3-dihydro-1,3-benzoxazol-5-yl)-2,4-pyrimidinediamine;
IV-44 N2-[4-(2-methoxyethyl)-2H-1,4-benzoxazin-3(4H)-one-7-yl]-5-methyl-N4-(2-oxo-2,3-dihydro-1,3-benzoxazol-5-yl)-2,4-pyrimidinediamine;
IV-45 N2-[2,2-dimethyl-4-(2-methoxyethyl)-2H-pyrido [3,2-b] [1,4]oxazin-3(4H)-one-7-yl]-5-methyl-N4-(2-oxo-2,3-dihydro-1,3-benzoxazol-5-yl)-2,4-pyrimidinediamine;
IV-46 5-methyl-N4-(2-oxo-2,3-dihydro-1,3-benzoxazol-5-yl)-N2-(2H-pyrido [3,2-b] [1,4]oxazin-3(4H)-one-7-yl)-2,4-pyrimidinediamine;
IV-47 5-methyl-N4-(2-oxo-2,3-dihydro-1,3-benzoxazol-5-yl)-N2-(2H-pyrido [3,2-b] [1,4]oxazin-3(4H)-one-6-yl)-2,4-pyrimidinediamine;
IV-48 5-methyl-N2-(3-methylindazol-6-yl)-N4-(2-oxo-2,3-dihydro-1,3-benzoxazol-5-yl)-2,4-pyrimidinediamine;
IV-49 5-methyl-N2-(3-methylindazol-5-yl)-N4-(2-oxo-2,3-dihydro-1,3-benzoxazol-5-yl)-2,4-pyrimidinediamine;
IV-50 N2-[2,2-dimethyl-2H-1,4-benzoxazin-3(4H)-one-7-yl]-5-methyl-N4-(2-oxo-2,3-dihydro-1,3-benzoxazol-5-yl)-2,4-pyrimidinediamine;
IV-51 5-(5-methyl-2-(6-methylpyridin-2-ylamino)pyrimidin-4-ylamino)benzo[d]oxazol-2(3H)-one;
IV-52 5-(5-methyl-2-(5-methylpyridin-2-ylamino)pyrimidin-4-ylamino)benzo[d]oxazol-2(3H)-one;
IV-53 5-[2-(Isoquinolin-6-ylamino)-5-methyl-pyrimidin-4-ylamino]-3H-benzooxazol-2-one;
IV-54 5-[5-Methyl-2-(naphthalen-2-ylamino)-pyrimidin-4-ylamino]-3H-benzooxazol-2-one;
IV-55 5-[2-(4-Methoxy-naphthalen-2-ylamino)-5-methyl-pyrimidin-4-ylamino]-3H-benzooxazol-2-one;
IV-56 5-[2-(4-Hydroxy-naphthalen-2-ylamino)-5-methyl-pyrimidin-4-ylamino]-3H-benzooxazol-2-one;
IV-57 5-[2-(Isoquinolin-7-ylamino)-5-methyl-pyrimidin-4-ylamino]-3H-benzooxazol-2-one;
IV-58 N2-(4-methoxypyridin-2-yl)-5-methyl-N4-(2-oxo-2,3-dihydro-1,3-benzoxazol-5-yl)-2,4-pyrimidinediamine;
IV-59 5-[5-Methyl-2-(2,4,6-trifluoro-phenylamino)-pyrimidin-4-ylamino]-3H-benzooxazol-2-one;
IV-60 5-(2-(2,6-dimethylphenylamino)-5-methylpyrimidin-4-ylamino)benzo[d]oxazol-2(3H)-one;
IV-61 5-[5-Methyl-2-(2,4,6-trimethyl-phenylamino)-pyrimidin-4-ylamino]-3H-benzooxazol-2-one;
IV-62 5-(2-(2-fluoro-6-methylphenylamino)-5-methylpyrimidin-4-ylamino)benzo[d]oxazol-2(3H)-one;
IV-63 N2-(3-fluoropyridin-4-yl)-5-methyl-N4-(2-oxo-2,3-dihydro-1,3-benzoxazol-5-yl)-2,4-pyrimidinediamine;
IV-64 N2-(3-fluoropyridin-4-yl)-5-methyl-N4-(2-oxo-2,3-dihydro-1,3-benzoxazol-5-yl)-2,4-pyrimidinediamine trifluoroacetic acid salt;
V-1: N-(1-(2-hydroxy-2-methylpropyl)-3-(pyridin-2-yl)-1H-pyrazol-4-yl)-5-(1-methyl-1H-pyrazol-4-yl)furan-2-carboxamide 2,2,2-trifluoroacetate;
V-2: N-(1-(2-hydroxy-2-methylpropyl)-3-(pyridin-2-yl)-1H-pyrazol-4-yl)-5-(1-methyl-1H-pyrazol-4-yl)furan-2-carboxamide;
V-3: N-(1-(2-hydroxy-2-methylpropyl)-3-(pyridin-2-yl)-1H-pyrazol-4-yl)-5-(1H-pyrazol-4-yl)furan-2-carboxamide;
V-4: tert-butyl 4-(5-((1-(2-ethoxyethyl)-3-(pyridin-2-yl)-1H-pyrazol-4-yl)carbamoyl)furan-2-yl)-1H-pyrazole-1-carboxylate;
V-5: N-(1-(2-methoxyethyl)-3-(pyridin-2-yl)-1H-pyrazol-4-yl)-5-(1H-pyrazol-4-yl)furan-2-carboxamide;
V-6: N-(1-(2-methoxyethyl)-3-(pyridin-2-yl)-1H-pyrazol-4-yl)-5-(3-methyl-1H-pyrazol-4-yl)furan-2-carboxamide formic acid;
V-9: N-(1-(2-methoxyethyl)-3-(pyridin-2-yl)-1H-pyrazol-4-yl)-5-(3-methyl-1H-pyrazol-4-yl)furan-2-carboxamide;
V-10: di-tert-butyl ((4-(5-((1-(2-methoxyethyl)-3-(pyridin-2-yl)-1H-pyrazol-4-yl)carbamoyl)furan-2-yl)-1H-pyrazol-1-yl)methyl) phosphate;
V-11: tert-butyl ((4-(5-((1-(2-methoxyethyl)-3-(pyridin-2-yl)-1H-pyrazol-4-yl)carbamoyl)furan-2-yl)-1H-pyrazol-1-yl)methyl) hydrogen phosphate;
V-12: (4-(5-((1-(2-methoxyethyl)-3-(pyridin-2-yl)-1H-pyrazol-4-yl)carbamoyl)furan-2-yl)-1H-pyrazol-1-yl)methyl dihydrogen phosphate;
V-13: N-(1-(2-methoxyethyl)-3-(pyridin-2-yl)-1H-pyrazol-4-yl)-5-(3-(trifluoromethyl)-1H-pyrazol-4-yl)furan-2-carboxamide;
V-14: sodium (4-(5-((1-(2-methoxyethyl)-3-(pyridin-2-yl)-1H-pyrazol-4-yl)carbamoyl)furan-2-yl)-1H-pyrazol-1-yl)methyl phosphate;
V-16: N-(1-(2-(2methoxyethoxy)ethyl)-3-(pyridin-2-yl)-1H-pyrazol-4-yl)-5-(1H-pyrazol-4-yl)furan-2-carboxamide;
V-17: N-(1-(2-(2-methoxyethoxy)ethyl)-3-(pyridin-2-yl)-1H-pyrazol-4-yl)-5-(1H-pyrazol-4-yl)furan-2-carboxamide hydrochloride salt;
V-18: N-(1-(2-(2-methoxyethoxy)ethyl)-3-(pyridin-2-yl)-1H-pyrazol-4-yl)-5-(3-methyl-1H-pyrazol-4-yl)furan-2-carboxamide;
V-19: 1-(isobutyryloxy)ethyl 4-(5-((1-(2-methoxyethyl)-3-(pyridin-2-yl)-1H-pyrazol-4-yl)carbamoyl)furan-2-yl)-1H-pyrazole-1-carboxylate;
V-20: tert-butyl (S)—(1-(4-(5-((1-(2-methoxyethyl)-3-(pyridin-2-yl)-1H-pyrazol-4-yl)carbamoyl)furan-2-yl)-1H-pyrazol-1-yl)-3-methyl-1-oxobutan-2-yl)carbamate;
V-21: 1-methylcyclopropyl 4-(5-((1-(2-methoxyethyl)-3-(pyridin-2-yl)-1H-pyrazol-4-yl)carbamoyl)furan-2-yl)-1H-pyrazole-1-carboxylate;
V-22: 1-((4-methoxybenzypoxy)-2-methylpropan-2-yl 4-(5-((1-(2-methoxyethyl)-3-(pyridin-2-yl)-1H-pyrazol-4-yl)carbamoyl)furan-2-yl)-1H-pyrazole-1-carboxylate;
V-23: 5-(1H-pyrazol-4-yl)-N-(3-(pyridin-2-yl)-1-(tetrahydro-2H-pyran-4-yl)-1H-pyrazol-4-yl)furan-2-carboxamide;
V-24: 5-(5-nitro-1H-pyrrol-3-yl)-N-(1-(propoxymethyl)-3-(pyridin-2-yl)-1H-pyrazol-4-yl)furan-2-carboxamide;
V-25: N-(1-(oxetan-3-yl)-3-(pyridin-2-yl)-1H-pyrazol-4-yl)-5-(1H-pyrazol-4-yl)furan-2-carboxamide;
V-26: 5-(1-methyl-1H-pyrazol-4-yl)-N-(1-(oxetan-3-yl)-3-(pyridin-2-yl)-1H-pyrazol-4-yl)furan-2-carboxamide;
V-27: N-(1-((1,3-trans)-3-ethoxycyclobutyl)-3-(pyridin-2-yl)-1H-pyrazol-4-yl)-5-(1H-pyrazol-4-yl)furan-2-carboxamide;
V-28: N-(1-((1,3-cis)-3-ethoxycyclobutyl)-3-(pyridin-2-yl)-1H-pyrazol-4-yl)-5-(1H-pyrazol-4-yl)furan-2-carboxamide;
V-29: N-(1-((1,3-cis)-3-ethoxycyclobutyl)-3-(pyridin-2-yl)-1H-pyrazol-4-yl)-5-(3-methyl-1H-pyrazol-4-yl)furan-2-carboxamide;
V-30: 5-(3-methyl-1H-pyrazol-4-yl)-N-(1-(oxetan-3-yl)-3-(pyridin-2-yl)-1H-pyrazol-4-yl)furan-2-carboxamide;
V-31: N-(1-((1,3-cis)-3-ethoxycyclobutyl)-3-(pyridin-2-yl)-1H-pyrazol-4-yl)-5-(1-methyl-1H-pyrazol-4-yl)furan-2-carboxamide;
V-32: N-(1-((1,3-cis)-3-hydroxycyclobutyl)-3-(pyridin-2-yl)-1H-pyrazol-4-yl)-5-(1H-pyrazol-4-yl)furan-2-carboxamide;
V-33: N-(1-((1s,3s)-3-(dimethylamino)cyclobutyl)-3-(pyridin-2-yl)-1H-pyrazol-4-yl)-5-(1H-pyrazol-4-yl)furan-2-carboxamide;
V-34: N-(1-((1s,3s)-3-(dimethylamino)cyclobutyl)-3-(pyridin-2-yl)-1H-pyrazol-4-yl)-5-(1H-pyrazol-4-yl)furan-2-carboxamide formate;
V-35: (4-(5-((1-((1,3-cis)-3-ethoxycyclobutyl)-3-(pyridin-2-yl)-1H-pyrazol-4-yl)carbamoyl)furan-2-yl)-1H-pyrazol-1-yl)methyl phosphate bis-sodium salt;
V-36: (4-(5-((1-((1s,3s)-3-ethoxycyclobutyl)-3-(pyridin-2-yl)-1H-pyrazol-4-yl)carbamoyl)furan-2-yl)-1H-pyrazol-1-yl)methyl dihydrogen phosphate;
V-37: N-(1-(2-ethoxyethyl)-3-(pyridin-2-yl)-1H-pyrazol-4-yl)-5-(1H-pyrazol-4-yl)furan-2-carboxamide formate;
V-38: N-(1-(2-ethoxyethyl)-3-(pyridin-2-yl)-1H-pyrazol-4-yl)-5-(1H-pyrazol-4-yl)furan-2-carboxamide;
V-39: N-(1-(2-ethoxyethyl)-3-(pyridin-2-yl)-1H-pyrazol-4-yl)-5-(1-ethyl-1H-pyrazol-4-yl)furan-2-carboxamide formate;
V-40: N-(1-(2-ethoxyethyl)-3-(pyridin-2-yl)-1H-pyrazol-4-yl)-5-(1-ethyl-1H-pyrazol-4-yl)furan-2-carboxamide;
V-41: N-(1-(2-ethoxyethyl)-3-(pyridin-2-yl)-1H-pyrazol-4-yl)-5-(3-(trifluoromethyl)-1H-pyrazol-4-yl)furan-2-carboxamide formate;
V-42: N-(1-(2-ethoxyethyl)-3-(pyridin-2-yl)-1H-pyrazol-4-yl)-5-(3-(trifluoromethyl)-1H-pyrazol-4-yl)furan-2-carboxamide;
V-43: N-(1-(2-ethoxyethyl)-3-(pyridin-2-yl)-1H-pyrazol-4-yl)-5-(1-isopentyl-1H-pyrazol-4-yl)furan-2-carboxamide formate;
V-44: N-(1-(2-ethoxyethyl)-3-(pyridin-2-yl)-1H-pyrazol-4-yl)-5-(1-isopentyl-1H-pyrazol-4-yl)furan-2-carboxamide;
V-45: N-(1-(2-ethoxyethyl)-3-(pyridin-2-yl)-1H-pyrazol-4-yl)-5-(1-methyl-1H-pyrazol-4-yl)furan-2-carboxamide formate;
V-46: N-(1-(2-ethoxyethyl)-3-(pyridin-2-yl)-1H-pyrazol-4-yl)-5-(1-methyl-1H-pyrazol-4-yl)furan-2-carboxamide;
V-47: 5-(1H-pyrazol-4-yl)-N-(3-(pyridin-2-yl)-1H-pyrazol-4-yl)furan-2-carboxamide;
V-48: 5-(1-((3-methyloxetan-3-yl)methyl)-1H-pyrazol-4-yl)-N-(1-((3-methyloxetan-3-yl)methyl)-3-(pyridin-2-yl)-1H-pyrazol-4-yl)furan-2-carboxamide formate;
V-49: 5-(1-((3-methyloxetan-3-yl)methyl)-1H-pyrazol-4-yl)-N-(1-((3-methyloxetan-3-yl)methyl)-3-(pyridin-2-yl)-1H-pyrazol-4-yl)furan-2-carboxamide;
V-52: 5-(1-(2-(2-methoxyethoxy)ethyl)-1H-pyrazol-4-yl)-N-(1-(2-(2-methoxyethoxy)ethyl)-3-(pyridin-2-yl)-1H-pyrazol-4-yl)furan-2-carboxamide formate;
V-53: 5-(1-(2-(2-methoxyethoxy)ethyl)-1H-pyrazol-4-yl)-N-(1-(2-(2-methoxyethoxy)ethyl)-3-(pyridin-2-yl)-1H-pyrazol-4-yl)furan-2-carboxamide;
V-54: (4-(5-((1-(2-ethoxyethyl)-3-(pyridin-2-yl)-1H-pyrazol-4-yl)carbamoyl)furan-2-yl)-1H-pyrazol-1-yl)methyl dihydrogen phosphate;
V-55: sodium (4-(5-((1-(2-ethoxyethyl)-3-(pyridin-2-yl)-1H-pyrazol-4-yl)carbamoyl)furan-2-yl)-1H-pyrazol-1-yl)methyl phosphate;
V-56: N-(1-(2-ethoxyethyl)-3-(pyridin-2-yl)-1H-pyrazol-4-yl)-5-(3-methyl-1H-pyrazol-4-yl)furan-2-carboxamide formate;
V-57: N-(1-(2-ethoxyethyl)-3-(pyridin-2-yl)-1H-pyrazol-4-yl)-5-(3-methyl-1H-pyrazol-4-yl)furan-2-carboxamide;
V-58: 5-(3,5-dimethyl-1H-pyrazol-4-yl)-N-(1-(2-ethoxyethyl)-3-(pyridin-2-yl)-1H-pyrazol-4-yl)furan-2-carboxamide formate;
V-59: 5-(3,5-dimethyl-1H-pyrazol-4-yl)-N-(1-(2-ethoxyethyl)-3-(pyridin-2-yl)-1H-pyrazol-4-yl)furan-2-carboxamide;
V-67: N-{1-Methyl-3-(pyridine-2-yl)-1H-pyrazol-4-yl}-5-(1H-pyrazol-4-yl)furan-2-carboxamide, formate salt;
V-68: 5-(1-Methyl-1H-pyrazol-4-yl)-N-{1-methyl-3-(pyridine-2-yl)-1H-pyrazol-4-yl}furan-2-carboxamide;
V-69: 5-(1-Methyl-1H-pyrazol-4-yl)-N-{1-methyl-3-(pyridine-2-yl)-1H-pyrazol-4-yl}furan-2-carboxamide, formate salt;
V-70: tert-Butyl-3-[4-{5-(1H-pyrazole-4-yl)furan-2-carboxamido}-3-(pyridine-2-yl)-1H-pyrazol-1-yl]azetidine-1-carboxylate, formate salt;
V-71: N-{1-(3-Methoxycyclobutyl)-3-(pyridine-2-yl)-1H-pyrazol-4-yl-}-5-(1H-pyrazol-4-yl)furan-2-carboxamide, formate salt, C is isomer;
V-72: N-{1-(3-Methoxycyclobutyl)-3-(pyridine-2-yl)-1H-pyrazol-4-yl-}-5-(1H-pyrazol-4-yl)furan-2-carboxamide, Cis isomer;
V-73: N-{1-(3-Benzyloxy)cyclobutyl)-3-(pyridine-2-yl)-1H-pyrazol-4-yl}-5-(1H-pyrazol-4-yl)furan-2-carboxamide, Trans isomer;
V-74: tert-Butyl-3-[4-{5-(1H-pyrazole-4-yl)furan-2-carboxamido}-3-(pyridine-2-yl)-1H-pyrazol-1-yl]azetidine-1-carboxylate;
V-75: N-(1-((1s,3s)-3-methoxycyclobutyl)-3-(pyridin-2-yl)-1H-pyrazol-4-yl)-5-(1H-pyrazol-4-yl)furan-2-carboxamide formate;
V-76: N-(1-((1s,3 s)-3-methoxycyclobutyl)-3-(pyridin-2-yl)-1H-pyrazol-4-yl)-5-(1H-pyrazol-4-yl)furan-2-carboxamide;
V-77: N-{1-Methyl-3-(pyridine-2-yl)-1H-pyrazol-4-yl}-5-(1H-pyrazol-4-yl)furan-2-carboxamide, free base;
V-78: N-{1-(Azetidin-3-yl)-3-(pyridine-2-yl)-1H-pyrazol-4-yl-}-5-(1H-pyrazol-4-yl)furan-2-carboxamide, TFA salt;
V-79: N-{1-(Azetidin-3-yl)-3-(pyridine-2-yl)-1H-pyrazol-4-yl-}-5-(1H-pyrazol-4-yl)furan-2-carboxamide;
V-80: Di-tert-butyl-[[4-{4-(5-((1-methyl-3-(pyridine-2-yl)-1H-pyrazol-4-yl)carbamoyl)furan-2-yl)-1H-pyrazol-1-yl}methyl]phosphate;
V-81: [4-{5-((1-Methyl-3-(pyridine-2-yl)-1H-pyrazol-4-yl)carbamoyl)furan-2yl}-1H-pyrazol-1-yl]methyl dihydrogen phosphate;
V-82: Sodium [4-{5-((1-Methyl-3-(pyridine-2-yl)-1H-pyrazol-4-yl)carbamoyl)furan-2-yl}-1H-pyrazol-1-yl]methyl phosphate;
V-83: N-{1-(1-Acetylazetidin-3-yl)-3-(pyridine-2-yl)-1H-pyrazol-4-yl}-5-(1H-pyrazol-4-yl)furan-4 2-carboxamide, free base;
V-84: 3-[4-{5-(1H-Pyrazol-4-yl)furan-2-carboxamido}-3-(pyridine-2-yl)-1H-pyrazol-1-yl]-N-(tert-butypazetidine-1-carboxamide, free base;
V-85: 3-[4-{5-(1H-Pyrazol-4-yl)furan-2-carboxamido}-3-(pyridine-2-yl)-1H-pyrazol-1-yl]-N-isopropylazetidine-1-carboxamide, free base;
V-86: 3-[4-{5-(1H-Pyrazol-4-yl)furan-2-carboxamido}-3-(pyridine-2-yl)-1H-pyrazol-1-yl]-N-propylazetidine-1-carboxamide, free base.
V-87: 3-[4-{5-(1H-Pyrazol-4-yl)furan-2-carboxamido}-3-(pyridine-2-yl)-1H-pyrazol-1-yl]-N-cyclopropylazetidine-1-carboxamide, formate salt;
V-88: 3-[4-{5-(1H-Pyrazol-4-yl)furan-2-carboxamido}-3-(pyridine-2-yl)-1H-pyrazol-1-yl]-N-cyclopropylazetidine-1-carboxamide;
V-89: N-[1-{1-(Cyclopropanecarbonyl)azetidin-3-yl}-3-(pyridine-2-yl)-1H-pyrazol-4-yl]-5-(1H-pyrazol-4-yl)furan-2-carboxamide, formate salt;
V-90: N-[1-{1-(Cyclopropanecarbonyl)azetidin-3-yl}-3-(pyridine-2-yl)-1H-pyrazol-4-yl]-5-(1H-pyrazol-4-yl)furan-2-carboxamide;
V-91: N-[1-{1-Pivaloylazetidin-3-yl}-3-(pyridine-2-yl)-1H-pyrazol-4-yl]-5-(1H-pyrazol-4-yl)furan-2-carboxamide, formate salt;
V-92: N-[1-{1-Pivaloylazetidin-3-yl}-3-(pyridine-2-yl)-1H-pyrazol-4-yl]-5-(1H-pyrazol-4-yl)furan-2-carboxamide;
V-93: 5-(1H-Pyrazol-4-yl)-N-{3-(pyridine-2-yl)-1-(pyrrolidine-1-carbonyl)azetidin-3-yl}-1H-pyrazol-4-yl)furan-2-carboxamide, formate salt;
V-94: 5-(1H-Pyrazol-4-yl)-N-{3-(pyridine-2-yl)-1-(pyrrolidine-1-carbonyl)azetidin-3-yl}-1H-pyrazol-4-yl)furan-2-carboxamide;
V-95: N-[1-{1-Isobutyrylazetidin-3-yl}-3-(pyridine-2-yl)-1H-pyrazol-4-yl]-5-(1H-pyrazol-4-yl)furan-2-carboxamide, formate salt;
V-96: N-[1-{1-Isobutyrylazetidin-3-yl}-3-(pyridine-2-yl)-1H-pyrazol-4-yl]-5-(1H-pyrazol-4-yl)furan-2-carboxamide;
V-97: N-(1H-Pyrazol-4-yl)-N-{3-(pyridine-2-yl)-1-{1-(2,2,2-trifluoroethypazetidin-3-yl}-1H-pyrazol-4-yl}furan-2-carboxamide, TFA salt;
V-98: N-(1H-Pyrazol-4-yl)-N-{3-(pyridine-2-yl)-1-{1-(2,2,2-trifluoroethypazetidin-3-yl}-1H-pyrazol-4-yl}furan-2-carboxamide;
V-99: N-[1-{1-Butyrylazetidin-3-yl}-3-(pyridine-2-yl)-1H-pyrazol-4-yl]-5-(1H-pyrazol-4-yl)furan-2-carboxamide, formate salt;
V-100: N-[1-{1-Butyrylazetidin-3-yl}-3-(pyridine-2-yl)-1H-pyrazol-4-yl]-5-(1H-pyrazol-4-yl)furan-2-carboxamide;
V-101: N-{1-(1-Methylazetidin-3-yl)-3-(pyridine-2-yl)-1H-pyrazol-4-yl}-5-(1H-pyrazol-4-yl)furan-2-carboxamide, formate salt;
V-102: N-{1-(1-Methylazetidin-3-yl)-3-(pyridine-2-yl)-1H-pyrazol-4-yl}-5-(1H-pyrazol-4-yl)furan-2-carboxamide;
V-103: N-[1-{1-(2,2-difluorocyclopropane-1-carbonyl)azetidin-3-yl}-3-(pyridine-2-yl)-1H-pyrazol-4-yl]-5-(1H-pyrazol-4-yl)furan-2-carboxamide, formate salt;
V-104: N-[1-{1-(2,2-difluorocyclopropane-1-carbonyl)azetidin-3-yl}-3-(pyridine-2-yl)-1H-pyrazol-4-yl]-5-(1H-pyrazol-4-yl)furan-2-carboxamide;
V-105: N-(1-methyl-3-(5-morpholinopyridin-2-yl)-1H-pyrazol-4-yl)-5-(1H-pyrazol-4-yl)furan-2-carboxamide;
V-106: N-(1-methyl-3-(5-(4-methylpiperazin-1-yl)pyridin-2-yl)-1H-pyrazol-4-yl)-5-(1H-pyrazol-4-yl)furan-2-carboxamide;
V-107: N-(3-(5-(2-hydroxy-2-methylpropoxy)pyridin-2-yl)-1-methyl-1H-pyrazol-4-yl)-5-(1H-pyrazol-4-yl)furan-2-carboxamide;
V-108: N-(1-methyl-3-(5-(oxetan-3-yloxy)pyridin-2-yl)-1H-pyrazol-4-yl)-5-(1H-pyrazol-4-yl)furan-2-carboxamide;
V-109: N-(3-(5-methoxypyridin-2-yl)-1-methyl-1H-pyrazol-4-yl)-5-(1H-pyrazol-4-yl)furan-2-carboxamide;
V-110: N-(1-isopropyl-3-(pyridin-2-yl)-1H-pyrazol-4-yl)-5-(1H-pyrazol-4-yl)furan-2-carboxamide;
V-111: N-(1-(2-morpholinoethyl)-3-(pyridin-2-yl)-1H-pyrazol-4-yl)-5-(1H-pyrazol-4-yl)furan-2-carboxamide;
V-112: N-(1-(2-(4-methylpiperazin-1-yl)ethyl)-3-(pyridin-2-yl)-1H-pyrazol-4-yl)-5-(1H-pyrazol-4-yl)furan-2-carboxamide;
V-113: 5-(1H-pyrazol-3-yl)-N-(3-(pyridin-2-yl)-1-(2-(2,2,2-trifluoroethoxy)ethyl)-1H-pyrazol-4-yl)furan-2-carboxamide;
V-114: N-(1-((1s,3s)-3-isopropoxycyclobutyl)-3-(pyridin-2-yl)-1H-pyrazol-4-yl)-5-(1H-pyrazol-4-yl)furan-2-carboxamide;
V-115: N-(1-(difluoromethyl)-3-(pyridin-2-yl)-1H-pyrazol-4-yl)-5-(1H-pyrazol-4-yl)furan-2-carboxamide;
V-116: N-(1-((1s,3s)-3-ethoxycyclobutyl)-3-(6-(trifluoromethyppyridin-2-yl)-1H-pyrazol-4-yl)-5-(1H-pyrazol-4-yl)furan-2-carboxamide;
V-117: 5-(1H-pyrazol-4-yl)-N-(3-(pyridin-2-yl)-1-(2,2,2-trifluoroethyl)-1H-pyrazol-4-yl)furan-2-carboxamide;
V-122: 5-(1-cyclobutyl-1H-pyrazol-4-yl)-N-(1-cyclobutyl-3-(pyridin-2-yl)-1H-pyrazol-4-yl)furan-2-carboxamide 2,2,2-trifluoroacetate;
V-123: 5-(1-cyclobutyl-1H-pyrazol-4-yl)-N-(1-cyclobutyl-3-(pyridin-2-yl)-1H-pyrazol-4-yl)furan-2-carboxamide;
V-124: N-(1-((1s,4s)-4-hydroxycyclohexyl)-3-(pyridin-2-yl)-1H-pyrazol-4-yl)-5-(1H-pyrazol-4-yl)furan-2-carboxamide formate;
V-125: N-(1-((1s,4s)-4-hydroxycyclohexyl)-3-(pyridin-2-yl)-1H-pyrazol-4-yl)-5-(1H-pyrazol-4-yl)furan-2-carboxamide;
V-126: N-(1-((1r,4r)-4-hydroxycyclohexyl)-3-(pyridin-2-yl)-1H-pyrazol-4-yl)-5-(1H-pyrazol-4-yl)furan-2-carboxamide formate;
V-127: N-(1-((1r,4r)-4-hydroxycyclohexyl)-3-(pyridin-2-yl)-1H-pyrazol-4-yl)-5-(1H-pyrazol-4-yl)furan-2-carboxamide;
V-128: 5-(1H-pyrazol-4-yl)-N-(3-(pyridin-2-yl)-1-(3-(2,2,2-trifluoroethoxy)cyclobutyl)-1H-pyrazol-4-yl)furan-2-carboxamide formate;
V-129: 5-(1H-pyrazol-4-yl)-N-(3-(pyridin-2-yl)-1-(3-(2,2,2-trifluoroethoxy)cyclobutyl)-1H-pyrazol-4-yl)furan-2-carboxamide;
V-130: N-(1-((1r,4r)-4-ethoxycyclohexyl)-3-(pyridin-2-yl)-1H-pyrazol-4-yl)-5-(1H-pyrazol-4-yl)furan-2-carboxamide formate;
V-131: N-(1-((1r,4r)-4-ethoxycyclohexyl)-3-(pyridin-2-yl)-1H-pyrazol-4-yl)-5-(1H-pyrazol-4-yl)furan-2-carboxamide;
V-132: N-(1-((1S,3R)-3-ethoxycyclopentyl)-3-(pyridin-2-yl)-1H-pyrazol-4-yl)-5-(1H-pyrazol-4-yl)furan-2-carboxamide formate;
V-133: N-(1-((1S,3R)-3-ethoxycyclopentyl)-3-(pyridin-2-yl)-1H-pyrazol-4-yl)-5-(1H-pyrazol-4-yl)furan-2-carboxamide;
V-134: N-(1-((1S,3R)-3-hydroxycyclopentyl)-3-(pyridin-2-yl)-1H-pyrazol-4-yl)-5-(1H-pyrazol-4-yl)furan-2-carboxamide formate;
V-135: N-(1-((1 S,3R)-3-hydroxycyclopentyl)-3-(pyridin-2-yl)-1H-pyrazol-4-yl)-5-(1H-pyrazol-4-yl)furan-2-carboxamide;
V-136: N-(1-((1S,3S)-3-hydroxycyclopentyl)-3-(pyridin-2-yl)-1H-pyrazol-4-yl)-5-(1H-pyrazol-4-yl)furan-2-carboxamide formate;
V-137: N-(1-((1S,3S)-3-hydroxycyclopentyl)-3-(pyridin-2-yl)-1H-pyrazol-4-yl)-5-(1H-pyrazol-4-yl)furan-2-carboxamide;
V-138: N-(1-((1s,3 s)-3-ethoxycyclobutyl)-3-(5-fluoropyridin-2-yl)-1H-pyrazol-4-yl)-5-(1H-pyrazol-4-yl)furan-2-carboxamide formate;
V-139: N-(1-((1s,3 s)-3-ethoxycyclobutyl)-3-(5-fluoropyridin-2-yl)-1H-pyrazol-4-yl)-5-(1H-pyrazol-4-yl)furan-2-carboxamide;
V-140: N-(1-((1 S,3R)-3-ethoxy-2-fluorocyclobutyl)-3-(pyridin-2-yl)-1H-pyrazol-4-yl)-5-(1H-pyrazol-4-yl)furan-2-carboxamide formate;
V-141: N-(1-((1 S,3R)-3-ethoxy-2-fluorocyclobutyl)-3-(pyridin-2-yl)-1H-pyrazol-4-yl)-5-(1H-pyrazol-4-yl)furan-2-carboxamide;
V-142: N-(1-((1s,3 s)-3-ethoxycyclobutyl)-3-(3-fluoropyridin-2-yl)-1H-pyrazol-4-yl)-5-(1H-pyrazol-4-yl)furan-2-carboxamide formate;
V-143: N-(1-((1s,3 s)-3-ethoxycyclobutyl)-3-(3-fluoropyridin-2-yl)-1H-pyrazol-4-yl)-5-(1H-pyrazol-4-yl)furan-2-carboxamide;
V-144: N-(1-((1s,3s)-3-ethoxycyclobutyl)-3-(6-fluoropyridin-2-yl)-1H-pyrazol-4-yl)-5-(1H-pyrazol-4-yl)furan-2-carboxamide formate;
V-145: N-(1-((1s,3s)-3-ethoxycyclobutyl)-3-(6-fluoropyridin-2-yl)-1H-pyrazol-4-yl)-5-(1H-pyrazol-4-yl)furan-2-carboxamide;
V-146: 5-(1H-pyrazol-4-yl)-N-(3-(pyridin-2-yl)-1-((1s,3 s)-3-(2,2,2-trifluoroethoxy)cyclobutyl)-1H-pyrazol-4-yl)furan-2-carboxamide formate;
V-147: 5-(1H-pyrazol-4-yl)-N-(3-(pyridin-2-yl)-1-((1s,3 s)-3-(2,2,2-trifluoroethoxy)cyclobutyl)-1H-pyrazol-4-yl)furan-2-carboxamide;
V-148: N-(1-((1s,3s)-3-ethoxycyclobutyl)-3-(4-fluoropyridin-2-yl)-1H-pyrazol-4-yl)-5-(1H-pyrazol-4-yl)furan-2-carboxamide formate;
V-149: N-(1-((1s,3s)-3-ethoxycyclobutyl)-3-(4-fluoropyridin-2-yl)-1H-pyrazol-4-yl)-5-(1H-pyrazol-4-yl)furan-2-carboxamide;
V-150: N-(3-(6-fluoropyridin-2-yl)-1-((1s,3 s)-3-(2,2,2-trifluoroethoxy)cyclobutyl)-1H-pyrazol-4-yl)-5-(1H-pyrazol-4-yl)furan-2-carboxamide formate;
V-151: N-(3-(6-fluoropyridin-2-yl)-1-((1s,3 s)-3-(2,2,2-trifluoroethoxy)cyclobutyl)-1H-pyrazol-4-yl)-5-(1H-pyrazol-4-yl)furan-2-carboxamide;
V-152: N-(3-(3-fluoropyridin-2-yl)-1-((1s,3 s)-3-(2,2,2-trifluoroethoxy)cyclobutyl)-1H-pyrazol-4-yl)-5-(1H-pyrazol-4-yl)furan-2-carboxamide formate;
V-153: N-(3-(3-fluoropyridin-2-yl)-1-((1s,3 s)-3-(2,2,2-trifluoroethoxy)cyclobutyl)-1H-pyrazol-4-yl)-5-(1H-pyrazol-4-yl)furan-2-carboxamide;
V-154: N-(1-((1r,4r)-4-ethoxycyclohexyl)-3-(3-fluoropyridin-2-yl)-1H-pyrazol-4-yl)-5-(1H-pyrazol-4-yl)furan-2-carboxamide formate;
V-155: N-(1-((1r,4r)-4-ethoxycyclohexyl)-3-(3-fluoropyridin-2-yl)-1H-pyrazol-4-yl)-5-(1H-pyrazol-4-yl)furan-2-carboxamide;
V-156: N-(3-(3,6-difluoropyridin-2-yl)-1-((1s,3 s)-3-ethoxycyclobutyl)-1H-pyrazol-4-yl)-5-(1H-pyrazol-4-yl)furan-2-carboxamide;
VI-1: N-(1-(2-hydroxy-2-methylpropyl)-3-(pyridin-2-yl)-1H-pyrazol-4-yl)-2-(1H-pyrazol-4-yl)thiazole-4-carboxamide;
VI-2: 1-(isobutyryloxy)ethyl 4-(4-((1-methyl-3-(pyridin-2-yl)-1H-pyrazol-4-yl)carbamoyl)thiazol-2-yl)-1H-pyrazole-1-carboxylate;
VI-3: tert-butyl (R)—(3-methyl-1-(4-(4-((1-methyl-3-(pyridin-2-yl)-1H-pyrazol-4-yl)carbamoyl)thiazol-2-yl)-1H-pyrazol-1-yl)-1-oxobutan-2-yl)carbamate;
VI-4: 2-(1-((5-methyl-2-oxo-1,3-dioxol-4-yl)methyl)-1H-pyrazol-4-yl)-N-(1-methyl-3-(pyridin-2-yl)-1H-pyrazol-4-yl)thiazole-4-carboxamide;
VI-5: 1-methylcyclopropyl 4-(4-((1-methyl-3-(pyridin-2-yl)-1H-pyrazol-4-yl)carbamoyl)thiazol-2-yl)-1H-pyrazole-1-carboxylate;
VI-6: 1-((4-methoxybenzypoxy)-2-methylpropan-2-yl 4-(4-((1-methyl-3-(pyridin-2-yl)-1H-pyrazol-4-yl)carbamoyl)thiazol-2-yl)-1H-pyrazole-1-carboxylate;
VI-7: diethyl ((4-(4-((1-methyl-3-(pyridin-2-yl)-1H-pyrazol-4-yl)carbamoyl)thiazol-2-yl)-1H-pyrazol-1-yl)methyl)phosphonate;
VI-8: sodium ((4-(4-((1-methyl-3-(pyridin-2-yl)-1H-pyrazol-4-yl)carbamoyl)thiazol-2-yl)-1H-pyrazol-1-yl)methyl)phosphonate;
VI-9: ((4-(4-((1-methyl-3-(pyridin-2-yl)-1H-pyrazol-4-yl)carbamoyl)thiazol-2-yl)-1H-pyrazol-1-yl)methyl)phosphonic acid;
VI-10: 2-(1H-pyrazol-4-yl)-N-(3-(pyridin-2-yl)-1-(tetrahydro-2H-pyran-4-yl)-1H-pyrazol-4-yl)thiazole-4-carboxamide;
VI-11: N-(1-((1,3-cis)-3-ethoxycyclobutyl)-3-(pyridin-2-yl)-1H-pyrazol-4-yl)-2-(1H-pyrazol-4-yl)thiazole-4-carboxamide;
VI-12: N-(1-((1,3-trans)-3-ethoxycyclobutyl)-3-(pyridin-2-yl)-1H-pyrazol-4-yl)-2-(1H-pyrazol-4-yl)thiazole-4-carboxamide;
VI-13: N-(1-((1,3-cis)-3-ethoxycyclobutyl)-3-(pyridin-2-yl)-1H-pyrazol-4-yl)-2-(1-methyl-1H-pyrazol-4-yl)thiazole-4-carboxamide;
VI-14: N-(1-((1,3-cis)-3-hydroxycyclobutyl)-3-(pyridin-2-yl)-1H-pyrazol-4-yl)-2-(1H-pyrazol-4-yl)thiazole-4-carboxamide;
VI-15: N-(1-((1s,3s)-3-(dimethylamino)cyclobutyl)-3-(pyridin-2-yl)-1H-pyrazol-4-yl)-2-(1H-pyrazol-4-yl)thiazole-4-carboxamide;
VI-16: (4-(4-((1-((1,3-cis)-3-ethoxycyclobutyl)-3-(pyridin-2-yl)-1H-pyrazol-4-yl)carbamoyl)thiazol-2-yl)-1H-pyrazol-1-yl)methyl phosphate bis-sodium salt;
VI-17: (4-(4-((1-((1s,3s)-3-ethoxycyclobutyl)-3-(pyridin-2-yl)-1H-pyrazol-4-yl)carbamoyl)thiazol-2-yl)-1H-pyrazol-1-yl)methyl dihydrogen phosphate;
VI-18: N-(1-(2-ethoxyethyl)-3-(pyridin-2-yl)-1H-pyrazol-4-yl)-2-(1H-pyrazol-4-yl)thiazole-4-carboxamide, formic acid salt;
VI-19: N-(1-(2-ethoxyethyl)-3-(pyridin-2-yl)-1H-pyrazol-4-yl)-2-(5-(trifluoromethyl)-1H-pyrazol-4-yl)thiazole-4-carboxamide, formic acid salt;
VI-20: N-(1-(2-ethoxyethyl)-3-(pyridin-2-yl)-1H-pyrazol-4-yl)-2-(5-(trifluoromethyl)-1H-pyrazol-4-yl)thiazole-4-carboxamide;
VI-21: N-(1-(2-ethoxyethyl)-3-(pyridin-2-yl)-1H-pyrazol-4-yl)-2-(3-methyl-1H-pyrazol-4-yl)thiazole-4-carboxamide, formic acid salt;
VI-22: N-(1-(2-ethoxyethyl)-3-(pyridin-2-yl)-1H-pyrazol-4-yl)-2-(3-methyl-1H-pyrazol-4-yl)thiazole-4-carboxamide;
VI-23: 2-(3,5-dimethyl-1H-pyrazol-4-yl)-N-(1-(2-ethoxyethyl)-3-(pyridin-2-yl)-1H-pyrazol-4-yl)thiazole-4-carboxamide, formic acid salt;
VI-24: 2-(3,5-dimethyl-1H-pyrazol-4-yl)-N-(1-(2-ethoxyethyl)-3-(pyridin-2-yl)-1H-pyrazol-4-yl)thiazole-4-carboxamide;
VI-25: N-(1-(2-ethoxyethyl)-3-(pyridin-2-yl)-1H-pyrazol-4-yl)-2-(1H-pyrazol-4-yl)thiazole-4-carboxamide;
VI-26: N-(1-methyl-3-(pyridin-2-yl)-1H-pyrazol-4-yl)-2-(1H-pyrazol-4-yl)thiazole-4-carboxamide;
VI-27: 2-(3-methyl-1H-pyrazol-4-yl)-N-(1-methyl-3-(pyridin-2-yl)-1H-pyrazol-4-yl)thiazole-4-carboxamide;
VI-28: N-(1-(2-methoxyethyl)-3-(pyridin-2-yl)-1H-pyrazol-4-yl)-2-(1H-pyrazol-4-yl)thiazole -4-carboxamide;
VI-29: N-(1-(2-methoxyethyl)-3-(pyridin-2-yl)-1H-pyrazol-4-yl)-2-(3-methyl-1H-pyrazol-4-yl)thiazole-4-carboxamide, formic acid salt;
VI-30: N-(1-(2-methoxyethyl)-3-(pyridin-2-yl)-1H-pyrazol-4-yl)-2-(3-methyl-1H-pyrazol-4-yl)thiazole-4-carboxamide;
VI-31: N-(1-(2-ethoxyethyl)-3-(pyridin-2-yl)-1H-pyrazol-4-yl)-2-(3-methyl-1H-pyrazol-4-yl)thiazole-4-carboxamide;
VI-32: 2-(1H-pyrazol-4-yl)-N-(3-(pyridin-2-yl)-1H-pyrazol-4-yl)thiazole-4-carboxamide formate;
VI-33: 2-(1H-pyrazol-4-yl)-N-(3-(pyridin-2-yl)-1H-pyrazol-4-yl)thiazole-4-carboxamide;
VI-34: N-(1-(oxetan-3-yl)-3-(pyridin-2-yl)-1H-pyrazol-4-yl)-2-(1H-pyrazol-4-yl)thiazole-4-carboxamide;
VI-35: (4-(4-((1-methyl-3-(pyridin-2-yl)-1H-pyrazol-4-yl)carbamoyl)thiazol-2-yl)-1H-pyrazol-1-yl)methyl dihydrogen phosphate;
VI-36: Sodium (4-(4-((1-methyl-3-(pyridin-2-yl)-1H-pyrazol-4-yl)carbamoyl)thiazol-2-yl)-1H-pyrazol-1-yl)methyl phosphate;
VI-37: N-(1-(2-(2-methoxyethoxy)ethyl)-3-(pyridin-2-yl)-1H-pyrazol-4-yl)-2-(1H-pyrazol-4-yl)thiazole-4-carboxamide;
VI-38: potassium (4-(4-((1-methyl-3-(pyridin-2-yl)-1H-pyrazol-4-yl)carbamoyl)thiazol-2-yl)-1H-pyrazol-1-yl)methyl phosphate;
VI-39: N-(1-(2-(2-methoxyethoxy)ethyl)-3-(pyridin-2-yl)-1H-pyrazol-4-yl)-2-(3-methyl-1H-pyrazol-4-yl)thiazole-4-carboxamide formate;
VI-40: N-(1-(2-(2-methoxyethoxy)ethyl)-3-(pyridin-2-yl)-1H-pyrazol-4-yl)-2-(3-methyl-1H-pyrazol-4-yl)thiazole-4-carboxamide;
VI-41: 2-(3-methyl-1H-pyrazol-4-yl)-N-(1-(oxetan-3-yl)-3-(pyridin-2-yl)-1H-pyrazol-4-yl)thiazole-4-carboxamide, formic acid salt;
VI-42: 2-(3-methyl-1H-pyrazol-4-yl)-N-(1-(oxetan-3-yl)-3-(pyridin-2-yl)-1H-pyrazol-4-yl)thiazole-4-carboxamide;
VI-43: 2-(1H-pyrazol-4-yl)-N-(3-(pyridin-2-yl)-1-(tetrahydrofuran-3-yl)-1H-pyrazol-4-yl)thiazole-4-carboxamide formate;
VI-44: 2-(1H-pyrazol-4-yl)-N-(3-(pyridin-2-yl)-1-(tetrahydrofuran-3-yl)-1H-pyrazol-4-yl)thiazole-4-carboxamide;
VI-45: 2-(3-methyl-1H-pyrazol-4-yl)-N-(3-(pyridin-2-yl)-1-((tetrahydro-2H-pyran-4-yl)methyl)-1H-pyrazol-4-yl)thiazole-4-carboxamide formate;
VI-46: 2-(3-methyl-1H-pyrazol-4-yl)-N-(3-(pyridin-2-yl)-1-((tetrahydro-2H-pyran-4-yl)methyl)-1H-pyrazol-4-yl)thiazole-4-carboxamide;
VI-47: N-(1-((3-(hydroxymethypoxetan-3-yl)methyl)-3-(pyridin-2-yl)-1H-pyrazol-4-yl)-2-(3-methyl-1H-pyrazol-4-yl)thiazole-4-carboxamide formate;
VI-48: N-(1-((3-(hydroxymethypoxetan-3-yl)methyl)-3-(pyridin-2-yl)-1H-pyrazol-4-yl)-2-(3-methyl-1H-pyrazol-4-yl)thiazole-4-carboxamide;
VI-49: N-(1-(2-(diethylamino)ethyl)-3-(pyridin-2-yl)-1H-pyrazol-4-yl)-2-(1H-pyrazol-4-yl)thiazole-4-carboxamide, formic acid salt;
VI-50: N-(1-(2-(diethylamino)ethyl)-3-(pyridin-2-yl)-1H-pyrazol-4-yl)-2-(1H-pyrazol-4-yl)thiazole-4-carboxamide;
VI-51: 2-(1-(4-methoxybenzyl)-1H-pyrazol-4-yl)-N-(1-(3-methoxycyclobutyl)-3-(pyridin-2-yl)-1H-pyrazol-4-yl)thiazole -4-carboxamide;
VI-52: N-(1-(2-fluoroethyl)-3-(pyridin-2-yl)-1H-pyrazol-4-yl)-2-(1-(4-methoxybenzyl)-1H-pyrazol-4-ypthiazole-4-carboxamide;
VI-53: 2-(1-(4-methoxybenzyl)-1H-pyrazol-4-yl)-N-(3-(pyridin-2-yl)-1H-pyrazol-4-yl)thiazole-4-carboxamide;
VI-54: tert-Butyl-3-[4-{2-(1H-pyrazole-4-yl)thiazole-2-carboxamido}-3-(pyridine-2-yl)-1H-pyrazol-1-yl]azetidine-1-carboxylate, free base;
VI-55: N-{1-(Azetidin-3-yl)-3-(pyridine-2-yl)-1H-pyrazol-4-yl}-2-(1H-pyrazol-4-yl)thiazole-4-carboxamide, TFA salt;
VI-56: N-{1-(Azetidin-3-yl)-3-(pyridine-2-yl)-1H-pyrazol-4-yl}-2-(1H-pyrazol-4-yl)thiazole-4-carboxamide;
VI-57: N-{1-(3-Methoxycyclobutyl)-3-(pyridine-2-yl)-1H-pyrazol-4-yl}-2-(1H-pyrazol-4-yl)thiazole-4-carboxamide, free base, Cis isomer;
VI-58: N-(3-(5-methoxypyridin-2-yl)-1-methyl-1H-pyrazol-4-yl)-2-(1H-pyrazol-4-yl)thiazole-4-carboxamide;
VI-59: N-(1-isopropyl-3-(pyridin-2-yl)-1H-pyrazol-4-yl)-2-(1H-pyrazol-4-yl)thiazole-4-carboxamide;
VI-60: N-(1-(2-morpholinoethyl)-3-(pyridin-2-yl)-1H-pyrazol-4-yl)-2-(1H-pyrazol-4-yl)thiazole-4-carboxamide;
VI-61: N-(1-(2-(4-methylpiperazin-1-yl)ethyl)-3-(pyridin-2-yl)-1H-pyrazol-4-yl)-2-(1H-pyrazol-4-yl)thiazole-4-carboxamide;
VI-65: N-(3-(3-fluoropyridin-2-yl)-1-((1s,3 s)-3-hydroxycyclobutyl)-1H-pyrazol-4-yl)-2-(1H-pyrazol-4-yl)thiazole-4-carboxamide;
VI-66: 2-(1H-pyrazol-3-yl)-N-(3-(pyridin-2-yl)-1-(2-(2,2,2-trifluoroethoxy)ethyl)-1H-pyrazol-4-yl)thiazole-4-carboxamide;
VI-71: N-(1-((1s,3s)-3-ethoxycyclobutyl)-3-(pyridin-2-yl)-1H-pyrazol-4-yl)-2-(5-fluoro-1-((2-(trimethylsilyl)ethoxy)methyl)-1H-pyrazol-4-yl)thiazole-4-carboxamide;
VI-72: N-(1-((1s,3s)-3-ethoxycyclobutyl)-3-(pyridin-2-yl)-1H-pyrazol-4-yl)-2-(5-fluoro-1H-pyrazol-4-yl)thiazole-4-carboxamide;
VI-73: N-(1-((1s,3s)-3-ethoxycyclobutyl)-3-(pyridin-2-yl)-1H-pyrazol-4-yl)-2-(5-fluoro-1H-pyrazol-4-yl)thiazole-4-carboxamide formate;
VI-76: N-(1-((1s,3s)-3-isopropoxycyclobutyl)-3-(pyridin-2-yl)-1H-pyrazol-4-yl)-2-(1H-pyrazol-4-yl)thiazole-4-carboxamide;
VI-77: potassium (4-(4-((1-((1s,3s)-3-ethoxycyclobutyl)-3-(pyridin-2-yl)-1H-pyrazol-4-yl)carbamoyl)thiazol-2-yl)-1H-pyrazol-1-yl)methyl phosphate;
VI-78: calcium (4-(4-((1-((1s,3s)-3-ethoxycyclobutyl)-3-(pyridin-2-yl)-1H-pyrazol-4-yl)carbamoyl)thiazol-2-yl)-1H-pyrazol-1-yl)methyl phosphate;
VI-79: N-(1-((1r,2r)-3-hydroxy-3-methylcyclobutyl)-3-(pyridin-2-yl)-1H-pyrazol-4-yl)-2-(1H-pyrazol-4-yl)thiazole-4-carboxamide;
VI-80: ammonium (4-(4-((1-((1s,3s)-3-ethoxycyclobutyl)-3-(pyridin-2-yl)-1H-pyrazol-4-yl)carbamoyl)thiazol-2-yl)-1H-pyrazol-1-yl)methyl phosphate;
VI-81: 5-amino-5-carboxypentan-1-aminium (4-(4-((1-((1s,3s)-3-ethoxycyclobutyl)-3-(pyridin-2-yl)-1H-pyrazol-4-yl)carbamoyl)thiazol-2-yl)-1H-pyrazol-1-yl)methyl phosphate;
VI-82: 1-(4-amino-4-carboxybutyl)guanidinium (4-(4-((1-((1s,3s)-3-ethoxycyclobutyl)-3-(pyridin-2-yl)-1H-pyrazol-4-yl)carbamoyl)thiazol-2-yl)-1H-pyrazol-1-yl)methyl phosphate;
VI-83: (4-(4-((1-((1s,3s)-3-ethoxycyclobutyl)-3-(pyridin-2-yl)-1H-pyrazol-4-yl)carbamoyl)thiazol-2-yl)-1H-pyrazol-1-yl)methyl dihydrogen phosphate;
VI-84: 1,3-dihydroxy-2-(hydroxymethyl)propan-2-aminium (4-(4-((1-((1s,3s)-3-ethoxycyclobutyl)-3-(pyridin-2-yl)-1H-pyrazol-4-yl)carbamoyl)thiazol-2-yl)-1H-pyrazol-1-yl)methyl hydrogen phosphate;
VI-85: triethylammonium (4-(4-((1-((1s,3s)-3-ethoxycyclobutyl)-3-(pyridin-2-yl)-1H-pyrazol-4-yl)carbamoyl)thiazol-2-yl)-1H-pyrazol-1-yl)methyl hydrogen phosphate;
VI-86: N-(1-((1s,3 s)-3-ethoxycyclobutyl)-3-(5-(trifluoromethyppyridin-2-yl)-1H-pyrazol-4-yl)-2-(1H-pyrazol-4-yl)thiazole-4-carboxamide;
VI-87: N-(1-(3-hydroxy-3-methylcyclobutyl)-3-(pyridin-2-yl)-1H-pyrazol-4-yl)-2-(1H-pyrazol-4-yl)thiazole-4-carboxamide;
VI-88: N-(1-(difluoromethyl)-3-(pyridin-2-yl)-1H-pyrazol-4-yl)-2-(1H-pyrazol-4-yl)thiazole-4-carboxamide;
VI-89: N-(1-((1s,3 s)-3-ethoxycyclobutyl)-3-(3-(trifluoromethyppyridin-2-yl)-1H-pyrazol-4-yl)-2-(1H-pyrazol-4-yl)thiazole-4-carboxamide;
VI-90: N-(1-((1s,3 s)-3-ethoxycyclobutyl)-3-(6-(trifluoromethyppyridin-2-yl)-1H-pyrazol-4-yl)-2-(1H-pyrazol-4-yl)thiazole-4-carboxamide;
VI-91: N-(1-((1s,3s)-3-ethoxycyclobutyl)-3-(pyridin-2-yl)-1H-pyrazol-4-yl)-2-(3-(trifluoromethyl)-1H-pyrazol-4-yl)thiazole-4-carboxamide;
VI-92: N-(1-((1s,3 s)-3-ethoxycyclobutyl)-3-(pyridin-2-yl)-1H-pyrazol-4-yl)-2-(3-methyl-1H-pyrazol-4-yl)thiazole-4-carboxamide;
VI-93: 2-(3,5-dimethyl-1H-pyrazol-4-yl)-N-(1-((1s,3s)-3-ethoxycyclobutyl)-3-(pyridin-2-yl)-1H-pyrazol-4-yl)thiazole-4-carboxamide;
VI-94: 2-(1H-pyrazol-4-yl)-N-(3-(pyridin-2-yl)-1-(2,2,2-trifluoroethyl)-1H-pyrazol-4-yl)thiazole-4-carboxamide;
VI-95: N-(1-(difluoromethyl)-3-(pyridin-2-yl)-1H-pyrazol-4-yl)-2-(3-(trifluoromethyl)-1H-pyrazol-4-yl)thiazole-4-carboxamide;
VI-96: N-(1-(difluoromethyl)-3-(pyridin-2-yl)-1H-pyrazol-4-yl)-2-(3-methyl-1H-pyrazol-4-yl)thiazole-4-carboxamide;
VI-97: N-(1-(difluoromethyl)-3-(pyridin-2-yl)-1H-pyrazol-4-yl)-2-(1-(2,2,2-trifluoroethyl)-1H-pyrazol-4-yl)thiazole-4-carboxamide;
VI-98: 2-(1-(difluoromethyl)-1H-pyrazol-4-yl)-N-(1-(difluoromethyl)-3-(pyridin-2-yl)-1H-pyrazol-4-yl)thiazole-4-carboxamide;
VI-99: N-(1-((1s,3s)-3-ethoxycyclobutyl)-3-(6-(trifluoromethyppyridin-2-yl)-1H-pyrazol-4-yl)-2-(3-methyl-1H-pyrazol-4-yl)thiazole-4-carboxamide;
VI-100: 2-(3-methyl-1H-pyrazol-4-yl)-N-(3-(pyridin-2-yl)-1-(2,2,2-trifluoroethyl)-1H-pyrazol-4-yl)thiazole-4-carboxamide;
VI-103: 2-(1-(4-methoxybenzyl)-1H-pyrazol-4-yl)-N-(3-(pyridin-2-yl)-1-(3,3,3-trifluoro-2-hydroxypropyl)-1H-pyrazol-4-ypthiazole-4-carboxamide formate;
VI-104: 2-(1-(4-methoxybenzyl)-1H-pyrazol-4-yl)-N-(3-(pyridin-2-yl)-1-(3,3,3-trifluoro-2-hydroxypropyl)-1H-pyrazol-4-ypthiazole-4-carboxamide;
VI-105: N-(1-(dimethylcarbamoyl)-3-(pyridin-2-yl)-1H-pyrazol-4-yl)-2-(1-(4-methoxybenzyl)-1H-pyrazol-4-yl)thiazole-4-carboxamide formate;
VI-106: N-(1-(dimethylcarbamoyl)-3-(pyridin-2-yl)-1H-pyrazol-4-yl)-2-(1-(4-methoxybenzyl)-1H-pyrazol-4-yl)thiazole-4-carboxamide;
VI-107: 2-(1-(4-methoxybenzyl)-1H-pyrazol-4-yl)-N-(3-(pyridin-2-yl)-1-(3,3,3-trifluoro-2-hydroxy-2-(trifluoromethyl)propyl)-1H-pyrazol-4-yl)thiazole-4-carboxamide formate;
VI-108: 2-(1-(4-methoxybenzyl)-1H-pyrazol-4-yl)-N-(3-(pyridin-2-yl)-1-(3,3,3-trifluoro-2-hydroxy-2-(trifluoromethyl)propyl)-1H-pyrazol-4-yl)thiazole-4-carboxamide;
VI-117: N-(1-(2-(diethylamino)ethyl)-3-(pyridin-2-yl)-1H-pyrazol-4-yl)-2-(1H-pyrazol-4-yl)thiazole-4-carboxamide;
VI-118: N-(1-(2-(2-fluoroethoxy)ethyl)-3-(pyridin-2-yl)-1H-pyrazol-4-yl)-2-(1H-pyrazol-4-yl)thiazole-4-carboxamide formate;
VI-119: N-(1-(2-(2-fluoroethoxy)ethyl)-3-(pyridin-2-yl)-1H-pyrazol-4-yl)-2-(1H-pyrazol-4-yl)thiazole-4-carboxamide;
VI-120: N-(1-benzyl-3-(pyridin-2-yl)-1H-pyrazol-4-yl)-2-(1H-pyrazol-4-yl)thiazole-4-carboxamide;
VI-121: N-(1-cyclobutyl-3-(pyridin-2-yl)-1H-pyrazol-4-yl)-2-(1H-pyrazol-4-yl)thiazole-4-carboxamide;
VI-122: N-(1-(2-(2,2-difluoroethoxy)ethyl)-3-(pyridin-2-yl)-1H-pyrazol-4-yl)-2-(1H-pyrazol-4-yl)thiazole-4-carboxamide;
VI-123: N-(1-(((1r,2r)-3-hydroxycyclobutyl)methyl)-3-(pyridin-2-yl)-1H-pyrazol-4-yl)-2-(1H-pyrazol-4-yl)thiazole-4-carboxamide formate;
VI-124: N-(1-(((1r,2r)-3-hydroxycyclobutyl)methyl)-3-(pyridin-2-yl)-1H-pyrazol-4-yl)-2-(1H-pyrazol-4-yl)thiazole-4-carboxamide;
VI-125: N-(1-(dimethylcarbamoyl)-3-(pyridin-2-yl)-1H-pyrazol-4-yl)-2-(1H-pyrazol-4-yl)thiazole-4-carboxamide formate;
VI-126: N-(1-(dimethylcarbamoyl)-3-(pyridin-2-yl)-1H-pyrazol-4-yl)-2-(1H-pyrazol-4-yl)thiazole-4-carboxamide;
VI-127: N-(1-((1s,3s)-3-(ethoxy-d5)cyclobutyl)-3-(pyridin-2-yl)-1H-pyrazol-4-yl)-2-(1H-pyrazol-4-yl)thiazole-4-carboxamide;
VI-128: N-(1-(diethylcarbamoyl)-3-(pyridin-2-yl)-1H-pyrazol-4-yl)-2-(1H-pyrazol-4-yl)thiazole-4-carboxamide;
VI-129: N-(1-(morpholine-4-carbonyl)-3-(pyridin-2-yl)-1H-pyrazol-4-yl)-2-(1H-pyrazol-4-yl)thiazole-4-carboxamide;
VI-130: N-(1-((1s,3s)-3-(2-fluoroethoxy)cyclobutyl)-3-(pyridin-2-yl)-1H-pyrazol-4-yl)-2-(1H-pyrazol-4-yl)thiazole-4-carboxamide;
VI-131: N-(1-(morpholine-4-carbonyl)-3-(pyridin-2-yl)-1H-pyrazol-4-yl)-2-(1H-pyrazol-4-yl)thiazole-4-carboxamide formate;
VI-132: N-(1-(3-fluorocyclobut-2-en-1-yl)-3-(pyridin-2-yl)-1H-pyrazol-4-yl)-2-(1H-pyrazol-4-yl)thiazole-4-carboxamide;
VI-133: N-(1-(3-fluorocyclobut-2-en-1-yl)-3-(pyridin-2-yl)-1H-pyrazol-4-yl)-2-(1H-pyrazol-4-yl)thiazole-4-carboxamide formate;
VI-134: N-(1-(3,3-difluorocyclobutyl)-3-(pyridin-2-yl)-1H-pyrazol-4-yl)-2-(1H-pyrazol-4-yl)thiazole-4-carboxamide formate;
VI-135: N-(1-(3,3-difluorocyclobutyl)-3-(pyridin-2-yl)-1H-pyrazol-4-yl)-2-(1H-pyrazol-4-yl)thiazole-4-carboxamide;
VI-140: N-(3-(3-fluoropyridin-2-yl)-1-(1,4-dioxaspiro[4.5]decan-8-yl)-1H-pyrazol-4-yl)-2-(1H-pyrazol-4-yl)thiazole-4-carboxamide;
VI-141: 2-(1H-pyrazol-4-yl)-N-(3-(pyridin-2-yl)-1-((1r,2r)-3-(2,2,2-trifluoroethoxy)cyclobutyl)-1H-pyrazol-4-yl)thiazole-4-carboxamide formate;
VI-142: 2-(1H-pyrazol-4-yl)-N-(3-(pyridin-2-yl)-1-((1r,2r)-3-(2,2,2-trifluoroethoxy)cyclobutyl)-1H-pyrazol-4-yl)thiazole-4-carboxamide;
VI-143: N-(1-((1r,4r)-4-hydroxycyclohexyl)-3-(pyridin-2-yl)-1H-pyrazol-4-yl)-2-(1H-pyrazol-4-yl)thiazole-4-carboxamide formate;
VI-144: N-(1-((1r,4r)-4-hydroxycyclohexyl)-3-(pyridin-2-yl)-1H-pyrazol-4-yl)-2-(1H-pyrazol-4-yl)thiazole-4-carboxamide;
VI-145: N-(1-((1r,4r)-4-ethoxycyclohexyl)-3-(pyridin-2-yl)-1H-pyrazol-4-yl)-2-(1H-pyrazol-4-yl)thiazole-4-carboxamide formate;
VI-146: N-(1-((1r,4r)-4-ethoxycyclohexyl)-3-(pyridin-2-yl)-1H-pyrazol-4-yl)-2-(1H-pyrazol-4-yl)thiazole-4-carboxamide;
VI-147: N-(1-((1S,3R)-3-ethoxycyclopentyl)-3-(pyridin-2-yl)-1H-pyrazol-4-yl)-2-(1H-pyrazol-4-yl)thiazole-4-carboxamide formate;
VI-148: N-(1-((1S,3R)-3-ethoxycyclopentyl)-3-(pyridin-2-yl)-1H-pyrazol-4-yl)-2-(1H-pyrazol-4-yl)thiazole-4-carboxamide;
VI-149: N-(1-((1S,3R)-3-hydroxycyclopentyl)-3-(pyridin-2-yl)-1H-pyrazol-4-yl)-2-(1H-pyrazol-4-yl)thiazole-4-carboxamide formate;
VI-150: N-(1-((1S,3R)-3-hydroxycyclopentyl)-3-(pyridin-2-yl)-1H-pyrazol-4-yl)-2-(1H-pyrazol-4-yl)thiazole-4-carboxamide;
VI-151: N-(1-((1S,3S)-3-hydroxycyclopentyl)-3-(pyridin-2-yl)-1H-pyrazol-4-yl)-2-(1H-pyrazol-4-yl)thiazole-4-carboxamide formate;
VI-152: N-(1-((1S,3S)-3-hydroxycyclopentyl)-3-(pyridin-2-yl)-1H-pyrazol-4-yl)-2-(1H-pyrazol-4-yl)thiazole-4-carboxamide;
VI-153: N-(1-((1s,3s)-3-ethoxycyclobutyl)-3-(5-fluoropyridin-2-yl)-1H-pyrazol-4-yl)-2-(1H-pyrazol-4-yl)thiazole-4-carboxamide formate;
VI-154: N-(1-((1s,3s)-3-ethoxycyclobutyl)-3-(5-fluoropyridin-2-yl)-1H-pyrazol-4-yl)-2-(1H-pyrazol-4-yl)thiazole-4-carboxamide;
VI-155: N-(1-((1S,3R)-3-ethoxy-2-fluorocyclobutyl)-3-(pyridin-2-yl)-1H-pyrazol-4-yl)-2-(1H-pyrazol-4-yl)thiazole-4-carboxamide formate;
VI-156: N-(1-((1S,3R)-3-ethoxy-2-fluorocyclobutyl)-3-(pyridin-2-yl)-1H-pyrazol-4-yl)-2-(1H-pyrazol-4-yl)thiazole-4-carboxamide;
VI-157: N-(1-((1s,3s)-3-ethoxycyclobutyl)-3-(3-fluoropyridin-2-yl)-1H-pyrazol-4-yl)-2-(1H-pyrazol-4-yl)thiazole-4-carboxamide;
VI-158: N-(1-((1s,3s)-3-ethoxycyclobutyl)-3-(4-fluoropyridin-2-yl)-1H-pyrazol-4-yl)-2-(1H-pyrazol-4-yl)thiazole-4-carboxamide formate;
VI-159: N-(1-((1s,3s)-3-ethoxycyclobutyl)-3-(4-fluoropyridin-2-yl)-1H-pyrazol-4-yl)-2-(1H-pyrazol-4-yl)thiazole-4-carboxamide;
VI-160: N-(1-((1s,3s)-3-ethoxycyclobutyl)-3-(6-fluoropyridin-2-yl)-1H-pyrazol-4-yl)-2-(1H-pyrazol-4-yl)thiazole-4-carboxamide;
VI-161: 2-(1H-pyrazol-4-yl)-N-(3-(pyridin-2-yl)-1-((1s,3s)-3-(2,2,2-trifluoroethoxy)cyclobutyl)-1H-pyrazol-4-yl)thiazole-4-carboxamide formate;
VI-162: 2-(1H-pyrazol-4-yl)-N-(3-(pyridin-2-yl)-1-((1 s,3 s)-3-(2,2,2-trifluoroethoxy)cyclobutyl)-1H-pyrazol-4-yl)thiazole -4-carboxamide;
VI-163: (4-(4-((1-((1 s,3 s)-3-ethoxycyclobutyl)-3-(3-fluoropyridin-2-yl)-1H-pyrazol-4-yl)carbamoyl)thiazol-2-yl)-1H-pyrazol-1-yl)methyl dihydrogen phosphate; \ VI-164: sodium (4-(4-((1-((1 s,3 s)-3-ethoxycyclobutyl)-3-(3-fluoropyridin-2-yl)-1H-pyrazol-4-yl)carbamoyl)thiazol-2-yl)-1H-pyrazol-1-yl)methyl phosphate;
VI-165: N-(3-(3-fluoropyridin-2-yl)-1-((1 s,3 s)-3-(2,2,2-trifluoroethoxy)cyclobutyl)-1H-pyrazol-4-yl)-2-(1H-pyrazol-4-yl)thiazole -4-carboxamide formate;
VI-166: N-(3-(3-fluoropyridin-2-yl)-1-((1 s,3 s)-3-(2,2,2-trifluoroethoxy)cyclobutyl)-1H-pyrazol-4-yl)-2-(1H-pyrazol-4-yl)thiazole-4-carboxamide;
VI-167: N-(3-(3-fluoropyridin-2-yl)-1-((1r,3 r)-3-(2,2,2-trifluoroethoxy)cyclobutyl)-1H-pyrazol-4-yl)-2-(1H-pyrazol-4-yl)thiazole -4-carboxamide formate;
VI-168: N-(3-(3-fluoropyridin-2-yl)-1-((1r,3 r)-3-(2,2,2-trifluoroethoxy)cyclobutyl)-1H-pyrazol-4-yl)-2-(1H-pyrazol-4-yl)thiazole -4-carboxamide;
VI-169: N-(1-((1r,4r)-4-ethoxycyclohexyl)-3-(3-fluoropyridin-2-yl)-1H-pyrazol-4-yl)-2-(1H-pyrazol-4-yl)thiazole -4-carboxamide;
VI-170: N-(3-(6-fluoropyridin-2-yl)-1-((1 s,3 s)-3-(2,2,2-trifluoroethoxy)cyclobutyl)-1H-pyrazol-4-yl)-2-(1H-pyrazol-4-yl)thiazole -4-carboxamide formate;
VI-171: N-(3-(6-fluoropyridin-2-yl)-1-((1 s,3 s)-3-(2,2,2-trifluoroethoxy)cyclobutyl)-1H-pyrazol-4-yl)-2-(1H-pyrazol-4-yl)thiazole-4-carboxamide;
VI-172: N-(3-(6-fluoropyridin-2-yl)-1-((1 s,3 s)-3-hydroxycyclobutyl)-1H-pyrazol-4-yl)-2-(1H-pyrazol-4-yl)thiazole -4-carboxamide;
VI-173: (4-(4-((1-((1s,3s)-3-ethoxycyclobutyl)-3-(6-fluoropyridin-2-yl)-1H-pyrazol-4-yl)carbamoyl)thiazol-2-yl)-1H-pyrazol-1-yl)methyl dihydrogen phosphate;
VI-174: N-(3-(3,6-difluoropyridin-2-yl)-1-((1 s,3 s)-3-ethoxycyclobutyl)-1H-pyrazol-4-yl)-2-(1H-pyrazol-4-yl)thiazole -4-carboxamide;
VI-175: N-(1-((1 s,4 s)-4-ethoxycyclohexyl)-3-(3-fluoropyridin-2-yl)-1H-pyrazol-4-yl)-2-(1H-pyrazol-4-yl)thiazole -4-carboxamide;
VI-176: N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide;
VI-177: N-(3-(3,6-difluoropyridin-2-yl)-1-((1 s,4 s)-4-ethoxycyclohexyl)-1H-pyrazol-4-yl)-2-(1H-pyrazol-4-yl)thiazole -4-carboxamide; or
VI-180: N-(3-(3,5-difluoropyridin-2-yl)-1-((1r,4r)-4-ethoxycyclohexyl)-1H-pyrazol-4-yl)-2-(1H-pyrazol-4-yl)thiazole -4-carboxamide.

14. The method of claim 1, wherein administering the compound ameliorates a sign or symptom of CRS, compared to the severity of the sign or symptom prior to administration of the compound.

15. The method of claim 14, wherein the sign or symptom is a fever.

16. The method of claim 1, wherein administering comprises:
administering to a subject that has previously be administered a first therapy for which CRS is a known, suspected, or potential side effect; or
administering to a subject who will be, or is concurrently being, administered a first therapy for which CRS is a known, suspected, or potential side effect.

17. The method of claim 16, wherein the first therapy comprises a cell therapy.

18. The method of claim 17, wherein the cell therapy comprises chimeric antigen receptor (CAR)-expressing therapy, a transgenic receptor therapy, or a combination thereof.

19. The method of claim 1, wherein administering the compound further comprises administering a second therapeutic agent.

20. The method of claim 19 wherein the second therapeutic agent is a steroid, an anti-inflammatory agent, an immunosuppressant, or a combination thereof.

21. The method of claim 20, wherein:
the steroid is alclomethasone, algestone, beclomethasone, betamethasone, budesonide, clobetasol, clobetasone, clocortolone, cloprednol, corticosterone, cortisone, cortivazol, deflazacort, desonide, desoximethasone, dexamethasone, diflorasone, diflucortolone, difluprednate, enoxolone, fluazacort, flucloronide, fludrocortisone, flumethasone, flunisolide, fluocinolone, fluocinonide, fluocortin, fluocortolone, fluorometholone, fluperolone, fluprednidene, fluprednisolone, flurandrenolide, fluticasone, formocortal, halcinonide, halobetasol, halometasone, halopredone, hydrocortamate, hydrocortisone, loteprednol etabonate, mazipredone, medrysone, meprednisone, methylprednisolone, mometasone, paramethasone, prednicarbate, prednisolone, prednisone, prednival, prednylidene, rimexolone, tixocortol, triamcinolone, or any combination thereof;
the anti-inflammatory agent is an aminosalicylate, cyclooxygenase inhibitor, diclofenac, etodolac, famotidine, fenoprofen, flurbiprofen, ketoprofen, ketorolac, ibuprofen, indomethacin, meclofenamate, mefenamic acid, meloxicam, nambumetone, naproxen, oxaprozin, piroxicam, salsalate, sulindac, tolmetin, or a combination thereof; or
the immunosuppressant is mercaptopurine, a corticosteroid, an alkylating agent, a calcineurin inhibitor, an inhibitor of inosine monophosphate dehydrogenase (IMPDH), an agents designed to suppress cellular immunity while leaving the recipient's humoral immunologic response intact, or a combination thereof.

22. The method of claim 20, wherein the second therapeutic is dexamethasone or prednisone, or a combination thereof.
